(19) United States
(12) Patent Application Publication
(10) Pub. No.: US 2020/0369665 A1
(43) Pub. Date: Nov. 26, 2020

(54) IMIDAZO[1,2-A]PYRAZINE MODULATORS OF THE ADENOSINE A2A RECEPTOR

(71) Applicant: RYVU THERAPEUTICS S.A., Kraków (PL)

(72) Inventors: Aneta BOBOWSKA (NÉE WITKOWSKA), Tarlów (PL); Michal GALEZOWSKI, Kraków (PL); Mateusz NOWAK, Kraków (PL); Claude COMMANDEUR, Kraków (PL); Joanna SZEREMETA-SPISAK, Sieradz (PL); Marcin NOWOGRODZKI, Kraków (PL); Alicja OBARA, Kielce (PL); Anna DZIELAK, Gliwice (PL); Iwona LOZINSKA, Legionowo (PL); Marcelina DUDEK (NÉE SEDLAK), Tarnobrzeg (PL); Anita ` JANIGA, Wlosienica (PL); Jacek REUS, Brójce (PL); Marek WRONOWSKI, Debowa Góra (PL); Magdalena ZASTAWNA, Kraków (PL); Adam RADZIMIERSKI, Kutno (PL); Mateusz SWIRSKI, Skawina (PL); Julian ZACHMANN, Munich (DE); Charles-Henry FABRITIUS, Kraków (PL); Roderick Alan PORTER, Baldock Herts Hertfordshire (GB); Joanna FOGT, Leszno (PL)

(73) Assignee: RYVU THERAPEUTICS S.A., Kraków (PL)

(21) Appl. No.: 16/624,071

(22) PCT Filed: Jun. 29, 2018
(86) PCT No.: PCT/EP2018/067701

§ 371 Date: Dec. 18, 2019

(30) Foreign Application Priority Data
Jun. 30, 2017 (EP) 17460038.7

(51) Int. Cl. C07D 487/04; A61K 31/4985; A61K 45/06; C07D 519/00; A61K 31/5377

(52) U.S. Cl. CPC C07D 487/04; A61K 31/4985; A61K 31/5377; C07D 519/00; A61K 45/06

(57) ABSTRACT

The present invention relates to the compound of formula (I) and salts, stereoisomers, tautomers, isotopologues, or N-oxides thereof. The present invention is further concerned with the use of such a compound or salt, stereoisomer, tautomer, isotopologues, or N-oxide thereof as medicament and a pharmaceutical composition comprising said compound.

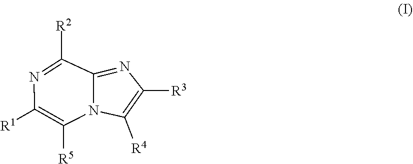

### FIELD OF THE INVENTION

The present invention relates to substituted imidazo[1,2-a]pyrazine compounds and salts, stereoisomers, tautomers, isotopologues, or N-oxides thereof. The present invention is further concerned with the use of substituted imidazo[1,2-a]pyrazine compounds or salts, stereoisomers, tautomers, isotopologues, or N-oxides thereof as medicament and a pharmaceutical composition comprising said compounds.

### BACKGROUND OF THE INVENTION

Cancer cells produce large quantities of mutated proteins (called neoantigens), which—when presented to the immune system—might lead to natural eradication of the tumor. However, to counteract this process, cancer cells produce also specific immunosuppressive metabolites that change the microenvironment and impair the function of immune cells. One of the key metabolites, which works this way is adenosine. Its immunosuppressive function is mediated by adenosine receptors, which are members of the G protein-coupled receptor (GPCR) family and possess seven transmembrane alpha helices. There are 4 subtypes of adenosine receptors described so far: A1, A2A, A2B, A3. They can be coupled to adenylate cyclase either positively (A2A, A2B) or negatively (A1, A3). Only forms A1 and A2A are heavily distributed in immune cells and mainly responsible for immunosuppression mediated by adenosine.

Stressed or injured tissues (i.e. tumor tissue) release endogenous ATP, which works as a proinflammatory agent. Hydrolysis of ATP by endonucleases (such as CD39 and CD73) leads to adenosine formation. Its binding to A2A and A2B receptors leads to cAMP elevation in immune cells and results in the activation of the CREB/ATF pathway (cAMP-responsive element (CRE)binding protein/activating transcription factor), the cell's main immunosuppressive mechanism [Grenz et al Antioxid Redox Signal 2011; 15:2221-34, 23. Fredholm et al Prog Neurobiol 2007; 83:263-76.,24.Sitkovsky Trends Immunol 2009; 30:102-8]. Its activation has been shown to either induce anergy of CD4+ T cells or their conversion into Tregs. This subpopulation of T cells is further activated by adenosine and produces immunosuppressive cytokines such as TGF-β and IL-10. Another group of immunosuppressing cells which respond to higher concentration of adenosine are MDSC, which undergo differentiation upon activation by this metabolite (Morrello et al Oncoimmunology. 2016 March; 5(3): e1108515). On the other hand, stimulation of adenosine might also lead to decreased cytotoxic activity, e.g. CD8+ lymphocytes lower their secretion of IL-2, Th1 cytokines and IFN-γ, while NK cells produce lower levels of GzmB, NKG2 d, CD69 and CD27 [Sitkovsky Trends Immunol 2009; 30:102-8]. Dendritic cells and macrophages are also affected by increased amounts of adenosine upon which they start to produce immunosuppressing agents such as IL-8, IL10 and TGFb, and stop production of immunostimulatory cytokines such as IL12, TNFa, IFNg. Adenosine also stimulates in macrophages the conversion of M1 to M2. [Allard et al Curr Opin Pharmacol. 2016 August; 29:7-16; Allard et al. Immunol Cell Biol 2017 April; 95(4):333-339.]

The above clearly shows that antagonizing adenosine receptors and thus reactivating the antitumor immune response may be an effective way of fighting all types of cancer. [Allard et al Curr Opin Pharmacol. 2016 August; 29:7-16]. It was shown in an allograft model that the use of A2A antagonists not only slows down the tumor growth but also blocks metastasis (in this particular case to lungs). Moreover, a strong synergistic correlation with checkpoint inhibitor antibodies has been demonstrated, likely improving the treatment [Iannone Am J Cancer Res. 2014 Mar. 1; 4(2):172-81, Cancer Immunol Res. 2015 May; 3(5):506-17; Allard et al. Immunol Cell Biol 2017 April; 95(4):333-339.].

Antagonists of the A2A receptor have already been shown as promising therapeutic for other diseases. The A2A receptor is abundant in the brain, where it plays a crucial role in the regulation of dopamine and glutamate release. Not surprisingly, the A2A receptor antagonists have been proposed useful in treatment of neurodegenerative disorders such as Parkinson's, Huntington's and Alzheimer's disease causing motor impairment, which can be improved by employment of A2A antagonists [Tuite P, et al., J. Expert Opin. Investig. Drugs. 2003; 12, 1335-52; Popoli P. et al. J Neurosci. 2002; 22, 1967-75; and Dall'lgna, et al., Experimental Neurology, 2007, 241-245]. Additionally, A2A antagonists may be used for the treatment of psychosis, stroke, extra pyramidal syndrome, e.g., dystonia, akathisia, pseudoparkinsonism and tardive dyskinesia (see Jenner P. J Neurol. 2000; 247 Supp2: 1 143-50) and attention related disorders such as attention deficit disorder (ADD) and attention deficit hyperactivity disorder (ADHD). Furthermore, A2A antagonists have been shown as useful agents for the treatment of amyotrophic lateral sclerosis (US 2007037033), cirrhosis, fibrosis and fatty liver (WO 01/058241) and the mitigation of addictive behavior (WO 06/009698). Adenosine A2A antagonists may be useful for the treatment and prevention of dermal fibrosis in diseases such as scleroderma (Chan et al. Arthritis & Rheumatism, 2006, 54(8), 2632-2642). Recently antagonists of A2A receptors were shown to possess the therapeutic potential as neuroprotectants (Stone T W. et al., Drag. Dev. Res. 2001, 52, 323-330), in the treatment of migraine (Kurokowa et al., 2009. Program No. 714.4/B101. 2009 Neuroscience Meeting Planner. Chicago, Ill.: Society for Neuroscience) and sleep disorders (Dunwiddie T V et al., Ann. Rev. Neurosci. 2001, 24, 31-55). WO 2017/098421 discloses inhibitors of CD73, wherein CD73 catalyzes the conversion of AMP to adenosine and is thought to be the major contributor to extracellular adenosine, in particular in the tumor microenvironment. CD73 inhibition results in decreased extracellular adenosine such that the activity of the A2A receptor is decreased, resulting in less (or no) immunosuppression—exactly the effect achieved with A2A receptor antagonists. It can thus be assumed that the diseases disclosed in WO 2017/098421 may also be treated by A2A antagonists.

In view of the above, there is the need for further compounds, which antagonize the A2A receptor in order to be capable of treating the afore-mentioned diseases.

### OBJECTS AND SUMMARY OF THE INVENTION

It is therefore an object of the present invention to provide compounds, which antagonize the adenosine A2A receptor.

It is another object of the present invention to provide compounds, which are capable of treating diseases, which are linked to the adenosine A2A receptor.

It is still another object of the present invention to provide compounds, which are suitable for the treatment of a disease selected from the group consisting of cancer, Parkinson's disease, Huntington's disease, Alzheimer's disease, psychosis, stroke, extra pyramidal syndrome (in particular dystonia, akathisia, pseudoparkinsonism and tardive dyskinesia), attention deficit disorder (ADD), attention deficit hyperactivity disorder (ADHD), amyotrophic lateral sclerosis, cirrhosis, fibrosis, fatty liver, addictive behavior, dermal fibrosis (in particular dermal fibrosis in scleroderma), sleep disorders, AIDS, autoimmune diseases, infections, atherosclerosis and ischemia-reperfusion injury.

In particular, it is an object of the present invention to provide compounds, which are suitable for the treatment of cancer, wherein this relates to the treatment of the tumor and the block of metastases.

The above objects can be achieved by the compounds of formula (I) as defined herein, and uses thereof.

The inventors of the present invention inter ala surprisingly found that the compounds of formula (I), as defined herein below (see first aspect), antagonize adenosine A2A receptor activity. Accordingly, the compounds of formula (I) or a pharmaceutical composition comprising a compound of formula (I), as defined herein below (see second aspect), can be used for the treatment of diseases linked to the adenosine A2A receptor, in particular the diseases given herein and most preferably cancer.

Therefore, in the first aspect A1, the present invention relates to a compound of formula (I)

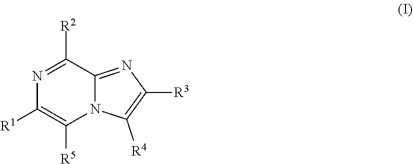

or a salt, stereoisomer, tautomer, isotopologue or N-oxide thereof,
wherein
R¹ is selected from the group consisting of a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said Nand/or S-atoms are independently oxidized or non-oxidized, and wherein each substitutable carbon or heteroatom in the aforementioned cyclic or bicyclic moieties is independently unsubstituted or substituted with one or more, same or different substituents R⁶;
R² is NH₂;
R³ is selected from the group consisting of
   (i) H, halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁸;
   (ii) C(═O)R²⁵, C(═O)OR²⁶, C(═O)SR²⁶, C(═O)N(R^{26a})(R^{26b}), OR²⁶, S(═O)ₙR²⁶, S(═O)ₙN(R^{26a})(R^{26b}), S(═O)ₘOR²⁶, N(R^{26a})(R^{26b}), N(R²⁶)C(═O)R²⁵, N(R²⁶)C(═O)OR²⁶, N(R²⁶)C(═O)N(R^{26a})(R^{26b}), N(R²⁶)S(═O)ₙ(R²⁶), N(R²⁶)S(═O)ₘN(R^{26a})(R^{26b}), and N(R²⁶)S(═O)ₘOR²⁶;
R⁴ is H;
R⁵ is selected from the group consisting of a 5- to 9-membered saturated, partially unsaturated or fully unsaturated carbocyclic or heterocyclic ring and a 9- to 12-membered saturated, partially unsaturated or fully unsaturated carbobicyclic or heterobicyclic ring, wherein said heterocyclic or heterobicyclic ring comprises one or more, same or different heteroatoms selected from O, N or S, wherein said Nand/or S-atoms are independently oxidized or non-oxidized, and wherein each substitutable carbon or heteroatom in the aforementioned cyclic or bicyclic moieties is independently unsubstituted or substituted with one or more, same or different substituents R¹⁷;
R⁶ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁷;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
   and/or two R⁶ present on one C-atom together form ═O;
R⁷ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R¹⁰;
   (ii) C(═O)R²¹, C(═O)OR²², C(═O)SR²², C(═O)N(R^{22a})(R^{22b}), OR²², S(═O)ₙR²², S(═O)ₙN(R^{22a})(R^{22b}), S(═O)ₘOR²², N(R^{22a})(R^{22b}), N(R²²)C(═O)R²¹, N(R²²)C(═O)OR²², N(R²²)C(═O)N(R^{22a})(R^{22b}), N(R²²)S(═O)ₙ(R²²), N(R²²)S(═O)ₘN(R^{22a})(R^{22b}), and N(R²²)S(═O)ₘOR²²;
R⁸ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁹;
   (ii) C(═O)R²⁷, C(═O)OR²⁸, C(═O)SR²⁸, C(═O)N(R^{28a})(R^{28b}), OR²⁸, S(═O)ₙR²⁸, S(═O)ₙN(R^{28a})(R^{28b}), S(═O)ₘOR²⁸, N(R^{28a})(R^{28b}), N(R²⁸)C(═O)R²⁷, N(R²⁸)C(═O)OR²⁸, N(R²⁸)C(═O)N(R^{28a})(R^{28b}), N(R²⁸)S(═O)ₙ(R²⁸), N(R²⁸)S(═O)ₘN(R^{28a})(R^{28b}), and N(R²⁸)S(═O)ₘOR²⁸;
R⁹ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R²⁹;
   (ii) C(═O)R³⁰, C(═O)OR³¹, C(═O)SR³¹, C(═O)N(R^{31a})(R^{31b}), OR³¹, S(═O)ₙR³¹, S(═O)ₙN(R^{31a})(R^{31b}), S(═O)ₘOR³¹, N(R^{31a})(R^{31b}), N(R³¹)C(═O)R³⁰, N(R³¹)C(═O)OR³¹, N(R³¹)C(═O)N(R^{31a})(R^{31b}), N(R³¹)S(═O)ₙ(R³¹), N(R³¹)S(═O)ₘN(R^{31a})(R^{31b}), and N(R³¹)S(═O)ₘOR³¹;
R¹⁰ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
R¹¹, R¹², R^{12a}, R^{12b} are independently selected from the group consisting of
   (i) H, C₁-C₆-alkyl, C₂-C₆-alkenyl, and C₂-C₆-alkynyl, wherein each substitutable carbon atom in the aforementioned moieties is independently unsubstituted or substituted with one or more, same or different substituents R¹³;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁴;
R¹³ is selected from the group consisting of
   (i) halogen, CN, NO₂, C₁-C₆-alkyl, C₁—C-haloalkyl, C₂-C₆-alkenyl, C₂-C₆-haloalkenyl, C₂-C₆-alkynyl, C₂-C₆-haloalkynyl, N(R^{15a})(R^{15b}), C(═O)NR^{15a}R^{15b}, S(═O)ₙNR^{15a}R^{15b}, OR¹⁵ and S(═O)ₙR¹⁵;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁴;
R¹⁴ is selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C(═O)R¹⁶, C(═O)OR¹⁵, C(═O)SR¹⁵, C(═O)N(R^{15a})(R^{15b}), OR¹⁵, S(═O)ₙR¹⁵, S(═O)ₙN(R^{15a})(R^{15b}), S(═O)ₘOR¹⁵, N(R^{15a})(R^{15b}), N(R¹⁵)C(═O)R¹⁶, N(R¹⁵)C(═O)OR¹⁵, N(R¹⁵)C(═O)N(R^{15a})(R^{15b}), N(R¹⁵)S(═O)ₙ(R¹⁵), N(R¹⁵)S(═O)ₘN(R^{15a})(R^{15b}), and N(R¹⁵)S(═O)ₘOR¹⁵;
R¹⁵, R^{15a}, R^{15b}, R¹⁶ are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
R¹⁷ is selected from the group consisting of
   (i) halogen, CN, NO₂, C₁-C₄-alkyl, C₂-C₄-alkenyl, C₂-C₄-alkynyl, and a 3- to 9-membered saturated, partially unsaturated or fully unsaturated carbocyclic or heterocyclic ring, wherein said heterocyclic ring comprises one or more, same or different heteroatoms selected from O, N or S, wherein said Nand/or S-atoms are independently oxidized or non-oxidized, and wherein each substitutable carbon or hetero-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
   and/or two R¹⁷ present on one C-atom together form ═O;
R¹⁸ is selected from the group consisting of
   (i) halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
   (ii) C(═O)R²³, C(═O)OR²⁴, C(═O)SR²⁴, C(═O)N(R^{24a})(R^{24b}), OR²⁴, S(═O)ₙR²⁴, S(═O)ₙN(R^{24a})(R^{24b}), S(═O)ₘOR²⁴, N(R^{24a})(R^{24b}), N(R²⁴)C(═O)R²³, N(R²⁴)C(═O)OR²⁴, N(R²⁴)C(═O)N(R^{24a})(R^{24b}), N(R²⁴)S(═O)ₙ(R²⁴), N(R²⁴)S(═O)ₘN(R^{24a})(R^{24b}), and N(R²⁴)S(═O)ₘOR²⁴;
R¹⁹, R²⁰, R^{20a}, R^{20b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
R²¹, R²², R^{22a}, R^{22b} are independently selected from the group consisting of
   (i) H, C₁-C₆-alkyl, C₂-C₆-alkenyl, and C₂-C₆-alkynyl, wherein each substitutable carbon atom in the aforementioned moieties is independently unsubstituted or substituted with one or more, same or different substituents R¹³;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁴;
R²³, R²⁴, R^{24a}, R^{24b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
R²⁵, R²⁶, R^{26a}, R^{26b} are independently selected from the group consisting of H, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R³²;
R²⁷, R²⁸, R^{28a}, R^{28b} are independently selected from the group consisting of H, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R³³;
R²⁹ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(═O)R³⁴, C(═O)OR³⁵, C(═O)SR³⁵, C(═O)N(R^{35a})(R^{35b}), OR³⁵, S(═O)ₙR³⁵, S(═O)ₙN(R^{35a})(R^{35b}), S(═O)ₘOR³⁵, N(R^{35a})(R^{35b}), N(R³⁵)C(═O)R³⁴, N(R³⁵)C(═O)OR³⁵, N(R³⁵)C(═O)N(R^{35a})(R^{35b}), N(R³⁵)S(═O)ₙ(R³⁵), N(R³⁵)S(═O)ₘN(R^{35a})(R^{35b}), and N(R³⁵)S(═O)ₘOR³⁵;
R³⁰, R³¹, R^{31a}, R^{31b} are independently selected from the group consisting of H, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R³⁷;
R³² is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R³⁸;
   (ii) C(═O)R³⁹, C(═O)OR⁴⁰, C(═O)SR⁴⁰, C(═O)N(R^{40a})(R^{40b}), OR⁴⁰, S(═O)ₙR⁴⁰, S(═O)ₙN(R^{40a})(R^{40b}), S(═O)ₘOR⁴⁰, N(R^{40a})(R^{40b}), N(R⁴⁰)C(═O)R³⁹, N(R⁴⁰)C(═O)OR⁴⁰, N(R⁴⁰)C(═O)N(R^{40a})(R^{40b}), N(R⁴⁰)S(═O)ₙ(R⁴⁰), N(R⁴⁰)S(═O)ₘN(R^{40a})(R^{40b}), and N(R⁴⁰)S(═O)ₘOR⁴⁰;
R³³ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁴¹;
   (ii) C(═O)R⁴², C(═O)OR⁴³, C(═O)SR⁴³, C(═O)N(R^{43a})(R^{43b}), OR⁴³, S(═O)ₙR⁴³, S(═O)ₙN(R^{43a})(R^{43b}), S(═O)ₘOR⁴³, N(R^{43a})(R^{43b}), N(R⁴³)C(═O)R⁴², N(R⁴³)C(═O)OR⁴³, N(R⁴³)C(═O)N(R^{43a})(R^{43b}), N(R⁴³)S(═O)ₙ(R⁴³), N(R⁴³)S(═O)ₘN(R^{43a})(R^{43b}), and N(R⁴³)S(═O)ₘOR⁴³;
R³⁴, R³⁵, R^{35a}, R^{35b} are independently selected from the group consisting of
   (i) H, C₁-C₆-alkyl, C₂-C₆-alkenyl, and C₂-C₆-alkynyl, wherein each substitutable carbon atom in the aforementioned moieties is independently unsubstituted or substituted with one or more, same or different substituents R³⁶;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁵²;
R³⁶ is selected from the group consisting of
   (i) halogen, CN, NO₂, C₁-C₆-alkyl, C₁—C-haloalkyl, C₂-C₆-alkenyl, C₂-C₆-haloalkenyl, C₂-C₆-alkynyl, C₂—C-haloalkynyl, N(R^{53a})(R^{53b}), C(═O)NR^{53a}R^{53b}, S(═O)ₙNR^{53a}R^{53b}, OR⁵³ and S(═O)ₙR⁵³;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁵²;
R³⁷ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, OH, O(C₁-C₄-alkyl), NH₂, NH(C₁-C₄-alkyl), and N(C₁-C₄-alkyl)(C₁-C₄-alkyl);
R³⁸ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁴⁴;
   (ii) C(═O)R⁴⁵, C(═O)OR⁴⁶, C(═O)SR⁴⁶, C(═O)N(R^{46a})(R^{46b}), OR⁴⁶, S(═O)ₙR⁴⁶, S(═O)ₙN(R^{46a})(R^{46b}), S(═O)ₘOR⁴⁶, N(R^{46a})(R^{46b}), N(R⁴⁶)C(═O)R⁴⁵, N(R⁴⁶)C(═O)OR⁴⁶, N(R⁴⁶)C(═O)N(R^{46a})(R^{46b}), N(R⁴⁶)S(═O)ₙ(R⁴⁶), N(R⁴⁶)S(═O)ₘN(R^{46a})(R^{46b}), and N(R⁴⁶)S(═O)ₘOR⁴⁶;
R³⁹, R⁴⁰, R^{40a}, R^{40b} are independently selected from the group consisting of H, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁴⁷;
R⁴¹ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, OH, O(C₁-C₄-alkyl), NH₂, NH(C₁-C₄-alkyl), and N(C₁-C₄-alkyl)(C₁-C₄-alkyl);
R⁴², R⁴³, R^{43a}, R^{43b} are independently selected from the group consisting of H, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁴⁸;
R⁴⁴ is selected from the group consisting of halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁴⁹;
R⁴⁵, R⁴⁶, R^{46a}, R^{46b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
R⁴⁷ is selected from the group consisting of halogen, CN, NO₂, OH, O(C₁-C₄-alkyl), NH₂, NH(C₁-C₄-alkyl), and N(C₁-C₄-alkyl)(C₁-C₄-alkyl), C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁵⁰;
R⁴⁸ is selected from the group consisting of halogen, CN, NO₂, OH, O(C₁-C₄-alkyl), NH₂, NH(C₁-C₄-alkyl), and N(C₁-C₄-alkyl)(C₁-C₄-alkyl), C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁵¹;
R⁴⁹, R⁵⁰, R⁵¹ are independently selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, OH, O(C₁-C₄-alkyl), NH₂, NH(C₁-C₄-alkyl), and N(C₁-C₄-alkyl)(C₁-C₄-alkyl);
R⁵² is selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C(═O)R⁵⁴, C(═O)OR⁵³, C(═O)SR⁵³, C(═O)N(R^{53a})(R^{53b}), OR⁵³, S(═O)ₙR⁵³, S(═O)ₙN(R^{53a})(R^{53b}), S(═O)ₘOR⁵³, N(R^{53a})(R^{53b}), N(R⁵³)C(═O)R⁵⁴, N(R⁵³)C(═O)OR⁵³, N(R⁵³)C(═O)N(R^{53a})(R^{53b}), N(R⁵³)S(═O)ₙ(R⁵³), N(R⁵³)S(═O)ₘN(R^{53a})(R^{53b}), and N(R⁵³)S(═O)ₘOR⁵³;
R⁵³, R^{53a}, R^{53b}, R⁵⁴ are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
and wherein
n is 0, 1 or 2; and
m is 1 or 2.

In a preferred embodiment, R¹ is selected from the group consisting of a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁶;
wherein R⁶ is selected from the group consisting of
   (i) halogen, CN, NO₂, C₁-C₆-alkyl, C₂-C₆-alkenyl, and C₂-C₆-alkynyl, wherein each substitutable carbon-atom in the aforementioned moieties is unsubstituted or substituted with one or more, same or different substituents R⁷;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
   and/or two R⁶ present on one C-atom together form ═O;
   wherein R⁷ is selected from the group consisting of
   (i) halogen, CN, NO₂, C₁-C₆-alkyl, C₂-C₆-alkenyl, and C₂-C₆-alkynyl,
   (ii) C(═O)R²¹, C(═O)OR²², C(═O)SR²², C(═O)N(R^{22a})(R^{22b}), OR²², S(═O)ₙR²², S(═O)ₙN(R^{22a})(R^{22b}), S(═O)ₘOR²², N(R^{22a})(R^{22b}), N(R²²)C(═O)R²¹, N(R²²)C(═O)OR²², N(R²²)C(═O)N(R^{22a})(R^{22b}), N(R²²)S(═O)ₙ(R²²), N(R²²)S(═O)ₘN(R^{22a})(R^{22b}), and N(R²²)S(═O)ₘOR²²;
   wherein R¹¹, R¹², R^{12a}, R^{12b}, R²¹, R²², R^{22a}, R^{22b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
   and wherein all other substituents have the meaning as defined above in the first aspect A1.

In another preferred embodiment, R1 is selected from the group consisting of a 5- to 6-membered fully unsaturated carbocyclic or heterocyclic ring and a 9- to 10-membered fully unsaturated carbobicyclic or heterobicyclic ring, wherein said heterocyclic or heterobicyclic ring comprises one or more, same or different heteroatoms selected from O, N or S, wherein said Nand/or S-atoms are independently oxidized or non-oxidized, and wherein each substitutable carbon or heteroatom in the aforementioned cyclic or bicyclic moieties is independently unsubstituted or substituted with one or more, same or different substituents R⁶;
wherein R⁶ is selected from the group consisting of
   (i) halogen, CN, NO₂, C₁-C₃-alkyl, C₂-C₃-alkenyl, and C₂-C₃-alkynyl, wherein each substitutable carbon-atom in the aforementioned moieties is unsubstituted or substituted with one or more, same or different substituents R⁷;
   (ii) C(═O)R¹¹, C(═O)OR¹², C(═O)N(R^{12a})(R^{12b}), OR¹², N(R^{12a})(R^{12b}), N(R¹²)C(═O)R¹¹, N(R¹²)C(═O)OR¹², and N(R¹²)C(═O)N(R^{12a})(R^{12b});
   and/or two R⁶ present on one C-atom together form ═O;
   wherein R⁷ is selected from the group consisting of
   (i) halogen, CN, NO₂, C₁-C₆-alkyl, C₂-C₆-alkenyl, and C₂-C₆-alkynyl,
   (ii) C(═O)R²¹, C(═O)OR²², C(═O)N(R^{22a})(R^{22b}), OR²², N(R^{22a})(R^{22b}), N(R²²)C(═O)R²¹, N(R²²)C(═O)OR²², and N(R²²)C(═O)N(R^{22a})(R^{22b});
   wherein R¹¹, R¹², R^{12a}, R^{12b}, R²¹, R²², R^{22a}, R^{22b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl; and
   wherein all other substituents have the meaning as defined above in the first aspect A1.

In another preferred embodiment, R¹ is a 5- to 6-membered fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, OH, O(C₁-C₄-alkyl), NH₂, NH(C₁-C₄-alkyl), and N(C₁-C₄-alkyl)(C₁-C₄-alkyl); and wherein all other substituents have the meaning as defined above in the first aspect A1.

In another preferred embodiment, R⁵ is selected from the group consisting of a 5- to 6-membered partially unsaturated or fully unsaturated carbocyclic or heterocyclic ring and a 9- to 10-membered partially unsaturated or fully unsaturated carbobicyclic or heterobicyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁷, and wherein all other substituents have the meaning as defined above in the first aspect A1.

In another preferred embodiment, R⁵ is selected from the group consisting of a 5- to 6-membered partially unsaturated or fully unsaturated carbocyclic or heterocyclic ring and a 9- to 10-membered partially unsaturated or fully unsaturated carbobicyclic or heterobicyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁷; wherein R¹⁷ is selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, C(═O)R¹⁹, C(═O)OR²⁰, C(═O)N(R^{20a})(R^{20b}), OR²⁰, N(R^{20a})(R^{20b}), N(R²⁰)C(═O)R¹⁹, N(R²⁰)C(═O)OR²⁰, and N(R²⁰)C(═O)N(R^{20a})(R^{20b}); and/or two R¹⁷ present on one C-atom together form ═O;

wherein R¹⁹, R²⁰, R^{20a}, R^{20b} are independently selected from the group consisting of H, C₁-C₂-alkyl, and C₁-C₂-haloalkyl; and
wherein all other substituents have the meaning as defined above in the first aspect A1.

In another preferred embodiment, R⁵ has the formula (S1)

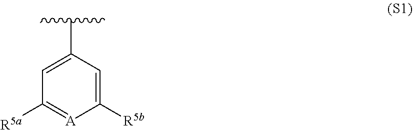

and wherein
   A is N or CR⁵;
R^{5a}, R^{5b}, R^{5c} are independently selected from the group consisting of
   (i) H, halogen, CN, NO₂, C₁-C₄-alkyl, C₂-C₄-alkenyl, and C₂-C₄-alkynyl, wherein each substitutable carbon-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
      with the proviso that at least one of R^{5a}, R^{5b}, R^{5c} is not H;
or
R^{5a} is selected from the group consisting of
   (i) H, halogen, CN, NO₂, C₁-C₄-alkyl, C₂-C₄-alkenyl, and C₂-C₄-alkynyl, wherein each substitutable carbon-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
      and
R^{5b} and R^{5c} together with the atoms to which they are attached form a 5- to 6-membered partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is independently unsubstituted or substituted with one or more, same or different substituents selected from the group consisting of
   (i) H, halogen, CN, NO₂, C₁-C₄-alkyl, C₂-C₄-alkenyl, and C₂-C₄-alkynyl, wherein each substitutable carbon-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}) and N(R²⁰)S(═O)ₘOR²⁰;
   and wherein all other substituents have the meaning as defined above in the first aspect A1.

In another preferred embodiment, R⁵ is selected from the group consisting of a 6-membered fully unsaturated carbocyclic or heterocyclic ring and a 9- to 10-membered fully unsaturated heterobicyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is independently substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, C(═O)R¹⁹, C(═O)OR²⁰, C(═O)N(R^{20a})(R²⁰), OR²⁰, N(R^{20a})(R²⁰), N(R²⁰)C(═O)R¹⁹, N(R²⁰)C(═O)OR²⁰, and N(R²⁰)C(═O)N(R^{20a})(R^{20b}); and wherein each substitutable carbon or heteroatom in the aforementioned bicyclic moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, C(═O)R¹⁹, C(═O)OR²⁰, C(═O)N(R^{20a})(R²⁰), OR²⁰, N(R^{20a})(R²⁰), N(R²⁰)C(═O)R¹⁹, N(R²⁰)C(═O)OR²⁰, and N(R²⁰)C(═O)N(R^{20a})(R^{20b});

wherein R¹⁹, R²⁰, R^{20a}, R^{20b} are independently selected from the group consisting of H, C₁-C₂-alkyl, and C₁-C₂-haloalkyl,
and wherein all other substituents have the meaning as defined above in the first aspect A1.

In another preferred embodiment, R³ is selected from the group consisting of
(i) H, C₁-C₆-alkyl, a 3- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁸;
(ii) C(═O)R²⁵, C(═O)OR²⁶, C(═O)N(R^{26a})(R^{26b}), OR²⁶, N(R^{26a})(R^{26b}), N(R²⁶)C(═O)R²⁵, N(R²⁶)C(═O)OR²⁶, and N(R²⁶)C(═O)N(R^{26a})(R^{26b});
wherein R⁸ is selected from the group consisting of
(i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁹;
(ii) C(═O)R²⁷, C(═O)OR²⁸, C(═O)N(R^{28a})(R^{28b}), OR²⁸, N(R^{28a})(R^{28b}), N(R²⁸)C(═O)R²⁷, N(R²⁸)C(═O)OR²⁸, and N(R²⁸)C(═O)N(R^{28a})(R²⁸);
wherein R⁹ is selected from the group consisting of
(i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R²⁹;
(ii) C(═O)R³⁰, C(═O)OR³¹, C(═O)N(R^{31a})(R^{31b}), OR³¹, N(R^{31a})(R^{31b}), N(R³¹)C(═O)R³⁰, N(R³¹)C(═O)OR³¹, N(R³¹)C(═O)N(R^{31a})(R^{31b});
wherein R²⁵, R²⁶, R^{26a}, R^{26b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³²;
wherein R²⁷, R²⁸, R^{28a}, R^{28b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³³;
wherein R²⁹ is selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein R³⁰, R³¹, R^{31a}, R^{31b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³⁷;
wherein R³² is selected from the group consisting of
   (i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³⁸;
   (ii) C(═O)R³⁹, C(═O)OR⁴⁰, C(═O)N(R^{40a})(R^{40b}), OR⁴⁰, N(R^{40a})(R^{40b}), N(R⁴⁰)C(═O)R³⁹, N(R⁴⁰)C(═O)OR⁴⁰, N(R⁴⁰)C(═O)N(R^{40a})(R^{40b});
wherein R³³ is selected from the group consisting of
   (i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴¹;
   (ii) C(═O)R⁴², C(═O)OR⁴³, C(═O)N(R^{43a})(R^{43b}), OR⁴³, N(R^{43a})(R^{43b}), N(R⁴³)C(═O)R⁴², N(R⁴³)C(═O)OR⁴³, N(R⁴³)C(═O)N(R^{43a})(R^{43b});
wherein R³⁷ is selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein R³⁸ is selected from the group consisting of
   (i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁴;
   (ii) C(═O)R⁴⁵, C(═O)OR⁴⁶, C(═O)N(R^{46a})(R^{46b}), OR⁴⁶, N(R^{46a})(R^{46b}), N(R⁴⁶)C(═O)R⁴⁵, N(R⁴⁶)C(═O)OR⁴⁶, N(R⁴⁶)C(═O)N(R^{46a})(R^{46b});
wherein R³⁹, R⁴⁰, R^{40a}, R^{40b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁷;
wherein R⁴¹ is selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein R⁴², R⁴³, R^{43a}, R^{43b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁸;
wherein R⁴⁴ is selected from the group consisting of halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁹;
wherein R⁴⁵, R⁴⁶, R^{46a}, R^{46b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
wherein R⁴⁷ is selected from the group consisting of halogen, CN, NO₂, OH, O(C₁-C₄-alkyl), NH₂, NH(C₁-C₄-alkyl), and N(C₁-C₄-alkyl)(C₁-C₄-alkyl), C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁵⁰
wherein R⁴⁸ is selected from the group consisting of halogen, CN, NO₂, OH, O(C₁-C₄-alkyl), NH₂, NH(C₁-C₄-alkyl), and N(C₁-C₄-alkyl)(C₁-C₄-alkyl), C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁵¹;
wherein R⁴⁹, R⁵⁰, R⁵¹ are independently selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
   and wherein all other substituents have the meaning as defined above in the first aspect A1.

In another embodiment, said compound is selected from the group consisting 4-{8-amino-6-phenylimidazo[1,2-a]pyrazin-5-yl}-2-chlorophenol; 4-[8-amino-6-(furan-2-yl)imidazo[1,2-a]pyrazin-5-yl]-2-chlorophenol; 5-(2,6-dimethylpyridin-4-yl)-6-phenylimidazo[1,2-a]pyrazin-8-amine; 5-[2-methyl-6-(trifluoromethyl)pyridin-4-yl]-6-phenylimidazo[1,2-a]pyrazin-8-amine; 4-[8-amino-6-(4-fluorophenyl)imidazo[1,2-a]pyrazin-5-yl]-2-chlorophenol; 6-(4-fluorophenyl)-5-[2-methyl-6-(trifluoromethyl)pyridin-4-yl]imidazo[1,2-a]pyrazin-8-amine; 5-(2,6-dimethylpyridin-4-yl)-6-(4-fluorophenyl)imidazo[1,2-a]pyrazin-8-amine; 6-(3-fluorophenyl)-5-[2-methyl-6-(trifluoromethyl)pyridin-4-yl]imidazo[1,2-a]pyrazin-8-amine; 3-{8-amino-5-[2-methyl-6-(trifluoromethyl)pyridin-4-yl]imidazo[1,2-a]pyrazin-6-yl}benzonitrile; 3-[8-amino-5-(3-chloro-4-hydroxyphenyl)imidazo[1,2-a]pyrazin-6-yl]benzonitrile; 4-[8-amino-6-(3-fluorophenyl)imidazo[1,2-a]pyrazin-5-yl]-2-chlorophenol; 4-[8-amino-2-(3-nitrophenyl)-6-phenylimidazo[1,2-a]pyrazin-5-yl]-2-chlorophenol; 5-(1H-indazol-5-yl)-6-phenylimidazo[1,2-a]pyrazin-8-amine; 4-{8-amino-6-phenylimidazo[1,2-a]pyrazin-5-yl}-2,6-dichlorophenol; 4-{8-amino-2-cyclohexyl-6-phenylimidazo[1,2-a]pyrazin-5-yl}-2-chlorophenol; 4-{8-amino-6-phenylimidazo[1,2-a]pyrazin-5-yl}-2-bromo-6-chlorophenol; 4-{8-amino-6-[4-(trifluoromethyl)phenyl]imidazo[1,2-a]pyrazin-5-yl}-2-chlorophenol; 3-[8-amino-5-(3-chloro-4-hydroxyphenyl)-6-phenylimidazo[1,2-a]pyrazin-2-yl]benzonitrile; 4-[8-amino-5-(3-chloro-4-hydroxyphenyl)imidazo[1,2-a]pyrazin-6-yl]benzonitrile; 4-{8-amino-6-phenylimidazo[1,2-a]pyrazin-5-yl}-N-methylpyridin-2-amine; 4-{8-amino-2,6-diphenylimidazo[1,2-a]pyrazin-5-yl}-2-chlorophenol; 4-[8-amino-6-(3-fluorophenyl)imidazo[1,2-a]pyrazin-5-yl]phenol; 4-[8-amino-6-(3-fluorophenyl)imidazo[1,2-a]pyrazin-5-yl]-N-methylpyridin-2-amine; 8-amino-5-(3-chloro-4-hydroxyphenyl)-6-phenylimidazo[1,2-a]pyrazine-2-carboxamide; 4-[8-amino-6-(4-fluorophenyl)imidazo[1,2-a]pyrazin-5-yl]-N-methylpyridin-2-amine; 6-(3-fluorophenyl)-5-(quinolin-6-yl)imidazo[1,2-a]pyrazin-8-amine; 5-(3,5-dichlorophenyl)-6-(4-fluorophenyl)imidazo[1,2-a]pyrazin-8-amine; 6-(2-fluorophenyl)-5-[2-methyl-6-(trifluoromethyl)pyridin-4-yl]imidazo[1,2-a]pyrazin-8-amine; 3-[8-amino-5-(3,5-dichlorophenyl)imidazo[1,2-a]pyrazin-6-yl]benzonitrile; 5-(3,5-dichlorophenyl)-6-phenylimidazo[1,2-a]pyrazin-8-amine; 4-{8-amino-6-[3-(trifluoromethyl)phenyl]imidazo[1,2-a]pyrazin-5-yl}-2-chlorophenol; 5-(3-chloro-1H-indazol-5-yl)-6-phenylimidazo[1,2-a]pyrazin-8-amine; 5-(2-chloro-6-methylpyridin-4-yl)-6-(3-fluorophenyl)imidazo[1,2-a]pyrazin-8-amine; 5-(2-chloro-6-methylpyridin-4-yl)-6-(4-fluorophenyl)imidazo[1,2-a]pyrazin-8-amine; 4-[8-amino-6-(3-fluorophenyl)imidazo[1,2-a]pyrazin-5-yl]benzamide; 5-(3-methyl-1H-indazol-5-yl)-6-phenylimidazo[1,2-a]pyrazin-8-amine; 5-(1H-indol-5-yl)-6-phenylimidazo[1,2-a]pyrazin-8-amine; 6-(3-fluorophenyl)-5-(pyridin-4-yl)imidazo[1,2-a]pyrazin-8-amine; 6-(1-methyl-1H-pyrazol-3-yl)-5-[2-methyl-6-(trifluoromethyl)pyridin-4-yl]imidazo[1,2-a]pyrazin-8-amine; 5-(3-fluoro-1H-indazol-5-yl)-6-phenylimidazo[1,2-a]pyrazin-8-amine; 4-[8-amino-6-(1-methyl-1H-pyrazol-3-yl)imidazo[1,2-a]pyrazin-5-yl]-2-chlorophenol; 5-(1-benzofuran-5-yl)-6-(3-fluorophenyl)imidazo[1,2-a]pyrazin-8-amine; 4-[8-amino-6-(2-fluorophenyl)imidazo[1,2-a]pyrazin-5-yl]-2-chlorophenol; 6-(3-fluorophenyl)-5-(2-methoxypyridin-4-yl)imidazo[1,2-a]pyrazin-8-amine; 5-(2-fluoro-6-methylpyridin-4-yl)-6-(3-fluorophenyl)imidazo[1,2-a]pyrazin-8-amine; 4-{8-amino-2-methyl-6-phenylimidazo[1,2-a]pyrazin-5-yl}-2-chlorophenol; 4-{8-amino-6-phenylimidazo[1,2-a]pyrazin-5-yl}-6-fluoro-N-methylpyridin-2-amine; 3-{8-amino-5-[2-(methylamino)pyridin-4-yl]imidazo[1,2-a]pyrazin-6-yl}benzonitrile; 5-[2-methyl-6-(trifluoromethyl)pyridin-4-yl]-6-(naphthalen-2-yl)imidazo[1,2-a]pyrazin-8-amine; 4-[8-amino-5-(3-chloro-4-hydroxyphenyl)-6-phenylimidazo[1,2-a]pyrazin-2-yl]benzonitrile; 5-(2,6-dimethylpyridin-4-yl)-6-(3-fluorophenyl)imidazo[1,2-a]pyrazin-8-amine; 4-[8-amino-6-(3-fluorophenyl)imidazo[1,2-a]pyrazin-5-yl]-2-chloro-6-methylphenol; 4-[8-amino-6-(3,5-difluorophenyl)imidazo[1,2-a]pyrazin-5-yl]-2-chlorophenol; 4-[8-amino-6-(3-fluorophenyl)imidazo[1,2-a]pyrazin-5-yl]-2,6-dichlorophenol; 5-(1,3-benzothiazol-5-yl)-6-(3-fluorophenyl)imidazo[1,2-a]pyrazin-8-amine; 4-[8-amino-6-(3-fluorophenyl)imidazo[1,2-a]pyrazin-5-yl]-2,6-dimethoxyphenol; 4-[8-amino-6-(4-fluorophenyl)imidazo[1,2-a]pyrazin-5-yl]-N,N,6-trimethylpyridin-2-amine; 4-[8-amino-6-(4-fluorophenyl)imidazo[1,2-a]pyrazin-5-yl]-N,6-dimethylpyridin-2-amine; 6-(3-fluorophenyl)-5-(1-methyl-1H-indazol-6-yl)imidazo[1,2-a]pyrazin-8-amine; 6-(3-fluorophenyl)-5-(8-fluoroquinolin-6-yl)imidazo[1,2-a]pyrazin-8-amine; 5-(1,3-benzothiazol-6-yl)-6-(3-fluorophenyl)imidazo[1,2-a]pyrazin-8-amine; 5,6-bis(1,3-benzothiazol-6-yl)imidazo[1,2-a]pyrazin-8-amine; 6-(3-fluorophenyl)-5-(7-methyl-1H-indazol-5-yl)imidazo[1,2-a]pyrazin-8-amine; 4-[8-amino-6-(3-fluoro-5-methoxyphenyl)imidazo[1,2-a]pyrazin-5-yl]-2-chlorophenol; 4-[8-amino-6-(3-fluorophenyl)imidazo[1,2-a]pyrazin-5-yl]-2,6-difluorophenol; ethyl 8-amino-6-(3-fluorophenyl)-5-[2-methyl-6-(trifluoromethyl)pyridin-4-yl]imidazo[1,2-a]pyrazine-2-carboxylate; 4-[8-amino-6-(3-fluorophenyl)imidazo[1,2-a]pyrazin-5-yl]-2-chloro-6-methoxyphenol; 8-amino-6-(3-fluorophenyl)-5-[2-methyl-6-(trifluoromethyl)pyridin-4-yl]imidazo[1,2-a]pyrazine-2-carboxamide; 6-(3-fluorophenyl)-5-(2-methylpyridin-4-yl)imidazo[1,2-a]pyrazin-8-amine; 8-amino-5-(3-chloro-4-hydroxyphenyl)-6-(3-fluorophenyl)imidazo[1,2-a]pyrazine-2-carboxylic acid; 5-(2,6-dichloropyridin-4-yl)-6-(3-fluorophenyl)imidazo[1,2-a]pyrazin-8-amine; 4-[8-amino-6-(3-fluorophenyl)imidazo[1,2-a]pyrazin-5-yl]-N,6-dimethylpyridin-2-amine; 6-(3-fluorophenyl)-5-{imidazo[1,2-a]pyridin-6-yl}imidazo[1,2-a]pyrazin-8-amine; 4-[8-amino-6-(3-fluorophenyl)imidazo[1,2-a]pyrazin-5-yl]-2,6-dimethylphenol; 8-amino-6-(3-fluorophenyl)-5-[2-methyl-6-(trifluoromethyl)pyridin-4-yl]imidazo[1,2-a]pyrazine-2-carboxylic acid; 8-amino-6-(3-fluorophenyl)-N,N-dimethyl-5-[2-methyl-6-(trifluoromethyl)pyridin-4-yl]imidazo[1,2-a]pyrazine-2-carboxamide; 8-amino-6-(3-fluorophenyl)-N-methyl-5-[2-methyl-6-(trifluoromethyl)pyridin-4-yl]imidazo[1,2-a]pyrazine-2-carboxamide; 5-(4-amino-3,5-dichlorophenyl)-6-(3-fluorophenyl)imidazo[1,2-a]pyrazin-8-amine; 6-(3-fluorophenyl)-5-(isoquinolin-6-yl)imidazo[1,2-a]pyrazin-8-amine; 6-(3-fluorophenyl)-5-(2-methoxy-6-methylpyridin-4-yl)imidazo[1,2-a]pyrazin-8-amine; 5-(1H-1,3-benzodiazol-6-yl)-6-(3-fluorophenyl)imidazo[1,2-a]pyrazin-8-amine; 6-(3-fluorophenyl)-5-(1-methyl-1H-1,3-benzodiazol-6-yl)imidazo[1,2-a]pyrazin-8-amine; ethyl 8-amino-5-(3-chloro-4-hydroxyphenyl)-6-(3-fluorophenyl)imidazo[1,2-a]pyrazine-2-carboxylate; 4-[8-amino-6-(4-fluorophenyl)imidazo[1,2-a]pyrazin-5-yl]-6-methyl-N-(propan-2-yl)pyridin-2-amine; 6-(3-fluorophenyl)-5-(4-methyl-1,3-benzothiazol-6-yl)imidazo[1,2-a]pyrazin-8-amine; 8-amino-6-(3-fluorophenyl)-5-[2-methyl-6-(trifluoromethyl)pyridin-4-yl]-N-(oxolan-3-yl)imidazo[1,2-a]pyrazine-2-carboxamide; 5-(8-chloroquinolin-6-yl)-6-(3-fluorophenyl)imidazo[1,2-a]pyrazin-8-amine; 4-[8-amino-6-(3-fluorophenyl)-2-(4-methylpiperazine-1-carbonyl)imidazo[1,2-a]pyrazin-5-yl]-2-chlorophenol; 6-[8-amino-6-(3-fluorophenyl)imidazo[1,2-a]pyrazin-5-yl]-1-methyl-2,3-dihydro-1H-1,3-benzodiazol-2-one; 5-[8-amino-6-(3-fluorophenyl)imidazo[1,2-a]pyrazin-5-yl]-2,3-dihydro-1H-indol-2-one; 6-(3-fluorophenyl)-5-(quinoxalin-6-yl)imidazo[1,2-a]pyrazin-8-amine; 5-(2-chloropyridin-4-yl)-6-(3-fluorophenyl)imidazo[1,2-a]pyrazin-8-amine; 5-(4-fluoro-1,3-benzothiazol-6-yl)-6-(3-fluorophenyl)imidazo[1,2-a]pyrazin-8-amine; 5-[8-amino-6-(3-fluorophenyl)imidazo[1,2-a]pyrazin-5-yl]-3-bromopyridin-2-ol; 5-[8-amino-6-(3-fluorophenyl)imidazo[1,2-a]pyrazin-5-yl]-1,2-dihydropyridin-2-one; 8-amino-5-(3-chloro-4-hydroxyphenyl)-6-(3-fluorophenyl)imidazo[1,2-a]pyrazine-2-carboxamide; 6-(3-fluorophenyl)-5-[2-methyl-6-(trifluoromethyl)pyridin-4-yl]-2-phenylimidazo[1,2-a]pyrazin-8-amine; 5-[8-amino-6-(3-fluorophenyl)imidazo[1,2-a]pyrazin-5-yl]-1,3-dimethyl-1,2-dihydropyridin-2-one; 6-(3-fluorophenyl)-5-[2-(pyrrolidin-1-yl)pyridin-4-yl]imidazo[1,2-a]pyrazin-8-amine; 4-[8-amino-2-(aminomethyl)-6-phenylimidazo[1,2-a]pyrazin-5-yl]-2-chlorophenol; 6-(3-fluorophenyl)-5-{pyrazolo[1,5-a]pyrimidin-6-yl}imidazo[1,2-a]pyrazin-8-amine; 6-(3-fluorophenyl)-5-(8-methylquinolin-6-yl)imidazo[1,2-a]pyrazin-8-amine; 6-(4-fluorophenyl)-5-(8-fluoroquinolin-6-yl)imidazo[1,2-a]pyrazin-8-amine; 6-(4-fluorophenyl)-5-(1-methyl-1H-1,2,3-benzotriazol-6-yl)imidazo[1,2-a]pyrazin-8-amine; 6-[8-amino-6-(3-fluorophenyl)imidazo[1,2-a]pyrazin-5-yl]-1,3-benzothiazol-2-amine; 6-(3-fluorophenyl)-5-(8-fluoroquinoxalin-6-yl)imidazo[1,2-a]pyrazin-8-amine; 6-(3-fluorophenyl)-5-{8-methylimidazo[1,2-a]pyridin-6-yl}imidazo[1,2-a]pyrazin-8-amine; 3-[8-amino-5-(8-fluoroquinolin-6-yl)imidazo[1,2-a]pyrazin-6-yl]benzonitrile; N-{4-[8-amino-6-(3-fluorophenyl)imidazo[1,2-a]pyrazin-5-yl]-6-methylpyridin-2-yl}acetamide; 6-(3-fluorophenyl)-5-[8-(trifluoromethyl)quinolin-6-yl]imidazo[1,2-a]pyrazin-8-amine; 6-(3-fluorophenyl)-5-(8-methoxyquinolin-6-yl)imidazo[1,2-a]pyrazin-8-amine; 6-(3-fluorophenyl)-5-(1,8-naphthyridin-3-yl)imidazo[1,2-a]pyrazin-8-amine; 6-(3-fluorophenyl)-5-(7-fluoroquinolin-6-yl)imidazo[1,2-a]pyrazin-8-amine; 5-(4-fluoro-1-methyl-1H-1,3-benzodiazol-6-yl)-6-(3-fluorophenyl)imidazo[1,2-a]pyrazin-8-amine; 6-(3-fluorophenyl)-5-(1,8-naphthyridin-4-yl)imidazo[1,2-a]pyrazin-8-amine; ethyl 8-amino-6-(3-fluorophenyl)-5-(8-fluoroquinolin-6-yl)imidazo[1,2-a]pyrazine-2-carboxylate; [8-amino-6-(3-fluorophenyl)-5-(8-fluoroquinolin-6-yl)imidazo[1,2-a]pyrazin-2-yl]methanol; 6-(3-fluorophenyl)-5-[2-methyl-6-(pyrrolidin-1-yl)pyridin-4-yl]imidazo[1,2-a]pyrazin-8-amine; 5-{8-fluoroimidazo[1,2-a]pyridin-6-yl}-6-(3-fluorophenyl)imidazo[1,2-a]pyrazin-8-amine; 2-[8-amino-5-(8-fluoroquinolin-6-yl)imidazo[1,2-a]pyrazin-6-yl]phenol; 6-(6-fluoropyridin-2-yl)-5-(8-fluoroquinolin-6-yl)imidazo[1,2-a]pyrazin-8-amine; 5-[8-amino-6-(3-fluorophenyl)imidazo[1,2-a]pyrazin-5-yl]-1-ethyl-1,2-dihydropyridin-2-one; 6-(3-fluorophenyl)-5-{1H-pyrrolo[2,3-b]pyridin-3-yl}imidazo[1,2-a]pyrazin-8-amine; 5-(5,8-difluoroquinolin-6-yl)-6-(3-fluorophenyl)imidazo[1,2-a]pyrazin-8-amine; 6-[8-amino-6-(3-fluorophenyl)imidazo[1,2-a]pyrazin-5-yl]quinolin-8-amine; ethyl 2-[8-amino-6-(3-fluorophenyl)-5-(8-fluoroquinolin-6-yl)imidazo[1,2-a]pyrazin-2-yl]acetate; 5-(7-fluoro-1H-indazol-5-yl)-6-(3-fluorophenyl)imidazo[1,2-a]pyrazin-8-amine; 6-(3-fluorophenyl)-5-(4-methylquinolin-6-yl)imidazo[1,2-a]pyrazin-8-amine; 6-[8-amino-6-(3-fluorophenyl)imidazo[1,2-a]pyrazin-5-yl]quinoline-8-carbonitrile; 5-{8-fluoro-[1,2,4]triazolo[1,5-a]pyridin-6-yl}-6-(3-fluorophenyl)imidazo[1,2-a]pyrazin-8-amine; 5-(4-fluoro-1H-1,3-benzodiazol-6-yl)-6-(3-fluorophenyl)imidazo[1,2-a]pyrazin-8-amine; 4-[8-amino-6-(3-fluorophenyl)imidazo[1,2-a]pyrazin-5-yl]-2-fluoro-6-(trifluoromethyl)phenol; 6-[8-amino-6-(3-fluorophenyl)imidazo[1,2-a]pyrazin-5-yl]isoquinolin-1-ol; 2-[8-amino-6-(3-fluorophenyl)-5-(8-fluoroquinolin-6-yl)imidazo[1,2-a]pyrazin-2-yl]acetic acid; 5-[8-amino-6-(3-fluorophenyl)imidazo[1,2-a]pyrazin-5-yl]-2,3-dihydro-1H-isoindol-1-one; 5-[8-amino-6-(3-fluorophenyl)imidazo[1,2-a]pyrazin-5-yl]-2,3-dihydro-1H-inden-1-one; 2-[8-amino-6-(3-fluorophenyl)-5-(8-fluoroquinolin-6-yl)imidazo[1,2-a]pyrazin-2-yl]ethan-1-ol; 2-[8-amino-6-(3-fluorophenyl)-5-(8-fluoroquinolin-6-yl)imidazo[1,2-a]pyrazin-2-yl]acetamide; 6-(3-fluorophenyl)-5-(4-methoxy-1,3-benzothiazol-6-yl)imidazo[1,2-a]pyrazin-8-amine; 6-[8-amino-6-(3-fluorophenyl)imidazo[1,2-a]pyrazin-5-yl]naphthalen-1-ol; 5-[4-fluoro-1-(propan-2-yl)-1H-1,3-benzodiazol-6-yl]-6-(3-fluorophenyl)imidazo[1,2-a]pyrazin-8-amine; 6-(3-fluorophenyl)-5-(3-methyl-1H-indazol-5-yl)imidazo[1,2-a]pyrazin-8-amine; 5-[8-amino-6-(3-fluorophenyl)imidazo[1,2-a]pyrazin-5-yl]-1-ethyl-3-fluoro-1,2-dihydropyridin-2-one; 6-[8-amino-6-(3-fluorophenyl)imidazo[1,2-a]pyrazin-5-yl]quinolin-3-amine; 5-(4-fluoro-1,3-benzothiazol-6-yl)-6-(4-fluorophenyl)imidazo[1,2-a]pyrazin-8-amine; 3-[8-amino-5-(4-fluoro-1,3-benzothiazol-6-yl)imidazo[1,2-a]pyrazin-6-yl]benzonitrile; 6-(4-fluorophenyl)-5-(4-methylquinolin-6-yl)imidazo[1,2-a]pyrazin-8-amine; 5-(1,3-benzothiazol-6-yl)-6-(4-fluorophenyl)imidazo[1,2-a]pyrazin-8-amine; 6-(3-fluorophenyl)-5-(quinazolin-6-yl)imidazo[1,2-a]pyrazin-8-amine; 5-(8-chloroquinolin-6-yl)-6-(4-fluorophenyl)imidazo[1,2-a]pyrazin-8-amine; 5-(8-fluoro-4-methylquinolin-6-yl)-6-(4-fluorophenyl)imidazo[1,2-a]pyrazin-8-amine; 6-(4-fluorophenyl)-5-(1-methyl-1H-1,3-benzodiazol-6-yl)imidazo[1,2-a]pyrazin-8-amine; 5-(4-fluoro-1-methyl-1H-1,3-benzodiazol-6-yl)-6-(4-fluorophenyl)imidazo[1,2-a]pyrazin-8-amine; 6-(3-fluorophenyl)-5-{3-methylimidazo[1,2-a]pyridin-6-yl}imidazo[1,2-a]pyrazin-8-amine; 3-(8-amino-5-{8-methylimidazo[1,2-a]pyridin-6-yl}imidazo[1,2-a]pyrazin-6-yl)benzonitrile; 3-[8-amino-5-(8-chloroquinolin-6-yl)imidazo[1,2-a]pyrazin-6-yl]benzonitrile; 3-[8-amino-5-(1,3-benzothiazol-6-yl)imidazo[1,2-a]pyrazin-6-yl]benzonitrile; 6-(3-fluorophenyl)-5-[5-(1H-pyrazol-5-yl)thiophen-2-yl]imidazo[1,2-a]pyrazin-8-amine; 3-[8-amino-5-(4-methylquinolin-6-yl)imidazo[1,2-a]pyrazin-6-yl]benzonitrile; 3-[8-amino-5-(8-methoxyquinolin-6-yl)imidazo[1,2-a]pyrazin-6-yl]benzonitrile; 3-[8-amino-5-(1-methyl-1H-1,3-benzodiazol-6-yl)imidazo[1,2-a]pyrazin-6-yl]benzonitrile; 6-[8-amino-6-(3-fluorophenyl)imidazo[1,2-a]pyrazin-5-yl]-N-methylquinolin-8-amine; 6-[8-amino-6-(3-fluorophenyl)imidazo[1,2-a]pyrazin-5-yl]-N,N-dimethylquinolin-8-amine; 5-(4-chloro-1,3-benzothiazol-6-yl)-6-(4-fluorophenyl)imidazo[1,2-a]pyrazin-8-amine; 8-amino-6-(3-cyanophenyl)-5-(quinolin-6-yl)imidazo[1,2-a]pyrazine-2-carboxamide; 2-[8-amino-6-(3-cyanophenyl)-5-(quinolin-6-yl)imidazo[1,2-a]pyrazin-2-yl]acetamide; 6-(4-fluorophenyl)-5-(2-methylquinolin-6-yl)imidazo[1,2-a]pyrazin-8-amine; 3-[8-amino-5-(8-aminoquinolin-6-yl)imidazo[1,2-a]pyrazin-6-yl]benzonitrile; 6-(4-fluorophenyl)-5-(3-fluoroquinolin-6-yl)imidazo[1,2-a]pyrazin-8-amine; 5-(3,5-dichloro-4-methoxyphenyl)-6-phenylimidazo[1,2-a]pyrazin-8-amine; 6-(4-fluorophenyl)-5-(2-fluoroquinolin-6-yl)imidazo[1,2-a]pyrazin-8-amine; methyl 5-[8-amino-6-(4-fluorophenyl)imidazo[1,2-a]pyrazin-5-yl]furan-2-carboxylate; 5-[8-amino-6-(4-fluorophenyl)imidazo[1,2-a]pyrazin-5-yl]-1,2-dihydropyridin-2-one; 3-[8-amino-5-(3-aminoquinolin-6-yl)imidazo[1,2-a]pyrazin-6-yl]benzonitrile; 3-(8-amino-5-{3-methylimidazo[1,2-a]pyridin-6-yl}imidazo[1,2-a]pyrazin-6-yl)benzonitrile; 3-[8-amino-5-(5-fluoroquinolin-6-yl)imidazo[1,2-a]pyrazin-6-yl]benzonitrile; 6-[8-amino-6-(4-fluorophenyl)imidazo[1,2-a]pyrazin-5-yl]imidazo[1,2-a]pyridine-3-carbonitrile; 5-[8-amino-6-(4-fluorophenyl)imidazo[1,2-a]pyrazin-5-yl]-1-methyl-1,2-dihydropyridin-2-one; 5-[8-amino-6-(3-fluorophenyl)imidazo[1,2-a]pyrazin-5-yl]-1-methyl-1,2-dihydropyridin-2-one; 6-[8-amino-6-(3-fluorophenyl)imidazo[1,2-a]pyrazin-5-yl]imidazo[1,2-a]pyridine-3-carbonitrile; 5-(4,8-dimethylquinolin-6-yl)-6-(4-fluorophenyl)imidazo[1,2-a]pyrazin-8-amine; 5-(1H-1,3-benzodiazol-6-yl)-6-(4-fluorophenyl)imidazo[1,2-a]pyrazin-8-amine; 6-(4-fluorophenyl)-5-{3-methylimidazo[1,2-a]pyridin-6-yl}imidazo[1,2-a]pyrazin-8-amine; 6-(4-fluorophenyl)-5-(4-methoxy-1H-1,3-benzodiazol-6-yl)imidazo[1,2-a]pyrazin-8-amine; 6-(4-fluorophenyl)-5-(3-methylquinolin-6-yl)imidazo[1,2-a]pyrazin-8-amine; 5-[8-amino-6-(4-fluorophenyl)imidazo[1,2-a]pyrazin-5-yl]-1-(difluoromethyl)-1,2-dihydropyridin-2-one; 1-{4-[8-amino-6-(4-fluorophenyl)imidazo[1,2-a]pyrazin-5-yl]pyridin-2-yl}ethan-1-one; 5-{8-fluoro-3-methylimidazo[1,2-a]pyridin-6-yl}-6-(3-fluorophenyl)imidazo[1,2-a]pyrazin-8-amine; 6-(4-fluorophenyl)-5-(4-methylquinazolin-6-yl)imidazo[1,2-a]pyrazin-8-amine; 4-{8-amino-2-cyclopropyl-6-phenylimidazo[1,2-a]pyrazin-5-yl}-2-chlorophenol; 6-[8-amino-6-(4-fluorophenyl)imidazo[1,2-a]pyrazin-5-yl]quinolin-3-amine; 6-(4-fluorophenyl)-5-{2-methylimidazo[1,2-a]pyridin-6-yl}imidazo[1,2-a]pyrazin-8-amine; 6-(4-fluorophenyl)-5-{imidazo[1,2-a]pyridin-6-yl}imidazo[1,2-a]pyrazin-8-amine; 6-(4-fluorophenyl)-5-(3-fluoropyridin-4-yl)imidazo[1,2-a]pyrazin-8-amine; 6-(3-fluorophenyl)-5-(3-fluoroquinolin-6-yl)imidazo[1,2-a]pyrazin-8-amine; 3-(8-amino-5-{imidazo[1,2-a]pyridin-6-yl}imidazo[1,2-a]pyrazin-6-yl)benzonitrile; 5-[8-amino-6-(4-fluorophenyl)imidazo[1,2-a]pyrazin-5-yl]-3-methyl-1,2-dihydropyridin-2-one; 3-[8-amino-5-(1H-1,3-benzodiazol-6-yl)imidazo[1,2-a]pyrazin-6-yl]benzonitrile; 6-(4-fluorophenyl)-5-{[1,2,4]triazolo[4,3-a]pyridin-6-yl}imidazo[1,2-a]pyrazin-8-amine; 5-{3-ethylimidazo[1,2-a]pyridin-6-yl}-6-(4-fluorophenyl)imidazo[1,2-a]pyrazin-8-amine; 6-(4-fluorophenyl)-5-{pyrazolo[1,5-a]pyridin-5-yl}imidazo[1,2-a]pyrazin-8-amine; 5-[8-amino-6-(4-fluorophenyl)imidazo[1,2-a]pyrazin-5-yl]-3-fluoro-1,2-dihydropyridin-2-one; 4-{8-amino-5-[2-methyl-6-(trifluoromethyl)pyridin-4-yl]imidazo[1,2-a]pyrazin-6-yl}benzonitrile; 4-[8-amino-5-(8-fluoroquinolin-6-yl)imidazo[1,2-a]pyrazin-6-yl]benzonitrile; 6-(3-fluorophenyl)-5-{1H,2H,3H-pyrrolo[2,3-b]pyridin-4-yl}imidazo[1,2-a]pyrazin-8-amine; 5-(8-fluoroquinolin-6-yl)-6-(pyridin-3-yl)imidazo[1,2-a]pyrazin-8-amine; 6-(2-fluoropyridin-4-yl)-5-(4-methylquinolin-6-yl)imidazo[1,2-a]pyrazin-8-amine; 5-[8-amino-5-(1-methyl-1H-1,3-benzodiazol-6-yl)imidazo[1,2-a]pyrazin-6-yl]-2-fluorobenzonitrile; 5-[8-amino-6-(5-methylfuran-2-yl)imidazo[1,2-a]pyrazin-5-yl]-1,2-dihydropyridin-2-one; 5-[8-amino-6-(1-methyl-1H-pyrazol-3-yl)imidazo[1,2-a]pyrazin-5-yl]-1,2-dihydropyridin-2-one; 5-[8-amino-5-(4-methylquinolin-6-yl)imidazo[1,2-a]pyrazin-6-yl]-2-fluorobenzonitrile; 6-(3-methoxyphenyl)-5-(4-methylquinolin-6-yl)imidazo[1,2-a]pyrazin-8-amine; 6-(1-methyl-1H-pyrazol-3-yl)-5-(4-methylquinolin-6-yl)imidazo[1,2-a]pyrazin-8-amine; 4-[8-amino-5-(4-methylquinolin-6-yl)imidazo[1,2-a]pyrazin-6-yl]-2-fluorobenzonitrile; 6-(5-methylfuran-2-yl)-5-(4-methylquinolin-6-yl)imidazo[1,2-a]pyrazin-8-amine; {4-[8-amino-5-(quinolin-6-yl)imidazo[1,2-a]pyrazin-6-yl]thiophen-2-yl}methanol; 6-(6-fluoropyridin-2-yl)-5-(4-methylquinolin-6-yl)imidazo[1,2-a]pyrazin-8-amine; 1-{4-[8-amino-5-(quinolin-6-yl)imidazo[1,2-a]pyrazin-6-yl]pyridin-2-yl}ethan-1-one; 5-(4-methylquinolin-6-yl)-6-(pyridin-4-yl)imidazo[1,2-a]pyrazin-8-amine; 4-[8-amino-5-(4-methylquinolin-6-yl)imidazo[1,2-a]pyrazin-6-yl]benzonitrile; 4-[8-amino-5-(8-chloroquinolin-6-yl)imidazo[1,2-a]pyrazin-6-yl]benzonitrile; 4-[8-amino-5-(quinolin-6-yl)imidazo[1,2-a]pyrazin-6-yl]benzonitrile; {5-[8-amino-5-(quinolin-6-yl)imidazo[1,2-a]pyrazin-6-yl]furan-2-yl}methanol; 4-[8-amino-5-(quinolin-6-yl)imidazo[1,2-a]pyrazin-6-yl]pyridine-2-carbonitrile; 5-(quinolin-6-yl)-6-(1,3-thiazol-4-yl)imidazo[1,2-a]pyrazin-8-amine; 6-(3-aminophenyl)-5-(quinolin-6-yl)imidazo[1,2-a]pyrazin-8-amine; 2-{4-[8-amino-5-(quinolin-6-yl)imidazo[1,2-a]pyrazin-6-yl]-1H-pyrazol-1-yl}ethan-1-ol; 5-[8-amino-5-(quinolin-6-yl)imidazo[1,2-a]pyrazin-6-yl]pyridine-3-carbonitrile; 5-[8-amino-5-(quinolin-6-yl)imidazo[1,2-a]pyrazin-6-yl]thiophene-2-carbonitrile; 6-(2-methylpyridin-4-yl)-5-(quinolin-6-yl)imidazo[1,2-a]pyrazin-8-amine; 5-[8-amino-5-(quinolin-6-yl)imidazo[1,2-a]pyrazin-6-yl]pyridin-2-amine; 6-(2-methoxypyridin-4-yl)-5-(quinolin-6-yl)imidazo[1,2-a]pyrazin-8-amine; 6-(3-methoxyphenyl)-5-(quinolin-6-yl)imidazo[1,2-a]pyrazin-8-amine; 6-(3-nitrophenyl)-5-(quinolin-6-yl)imidazo[1,2-a]pyrazin-8-amine; 6-(6-methoxypyridin-3-yl)-5-(quinolin-6-yl)imidazo[1,2-a]pyrazin-8-amine; methyl 5-[8-amino-5-(quinolin-6-yl)imidazo[1,2-a]pyrazin-6-yl]furan-2-carboxylate; 5-[8-amino-5-(quinolin-6-yl)imidazo[1,2-a]pyrazin-6-yl]-3-methylpyridine-2-carbonitrile; 3-[8-amino-5-(quinolin-6-yl)imidazo[1,2-a]pyrazin-6-yl]phenol; 5-(8-fluoroquinolin-6-yl)-6-(furan-2-yl)imidazo[1,2-a]pyrazin-8-amine; 6-(4-methoxyphenyl)-5-(quinolin-6-yl)imidazo[1,2-a]pyrazin-8-amine; 6-(6-fluoropyridin-2-yl)-5-(quinolin-6-yl)imidazo[1,2-a]pyrazin-8-amine; 6-(pyridin-4-yl)-5-(quinolin-6-yl)imidazo[1,2-a]pyrazin-8-amine; 5-(8-fluoroquinolin-6-yl)-6-(6-methoxypyridin-3-yl)imidazo[1,2-a]pyrazin-8-amine; 6-(6-fluoropyridin-3-yl)-5-(8-fluoroquinolin-6-yl)imidazo[1,2-a]pyrazin-8-amine; 6-(3,4-difluorophenyl)-5-(8-fluoroquinolin-6-yl)imidazo[1,2-a]pyrazin-8-amine; 5-(8-fluoroquinolin-6-yl)-6-[4-(trifluoromethyl)phenyl]imidazo[1,2-a]pyrazin-8-amine; 6-(furan-2-yl)-5-(quinolin-6-yl)imidazo[1,2-a]pyrazin-8-amine; 6-(5-methylfuran-2-yl)-5-(quinolin-6-yl)imidazo[1,2-a]pyrazin-8-amine; 6-(pyridin-3-yl)-5-(quinolin-6-yl)imidazo[1,2-a]pyrazin-8-amine; 6-(1-methyl-1H-pyrazol-3-yl)-5-(quinolin-6-yl)imidazo[1,2-a]pyrazin-8-amine; {3-[8-amino-5-(quinolin-6-yl)imidazo[1,2-a]pyrazin-6-yl]phenyl}methanol; 6-(5-fluoropyridin-3-yl)-5-(quinolin-6-yl)imidazo[1,2-a]pyrazin-8-amine; 6-(6-fluoropyridin-3-yl)-5-(quinolin-6-yl)imidazo[1,2-a]pyrazin-8-amine; 6-(4-fluorophenyl)-5-(quinolin-6-yl)imidazo[1,2-a]pyrazin-8-amine; 5-(quinolin-6-yl)-6-[3-(trifluoromethyl)phenyl]imidazo[1,2-a]pyrazin-8-amine; 6-(3-aminophenyl)-5-(8-fluoroquinolin-6-yl)imidazo[1,2-a]pyrazin-8-amine; 3-[8-amino-5-(8-fluoroquinolin-6-yl)imidazo[1,2-a]pyrazin-6-yl]phenol; 6-(1,3-benzothiazol-6-yl)-5-(8-fluoroquinolin-6-yl)imidazo[1,2-a]pyrazin-8-amine; 5-(8-fluoroquinolin-6-yl)-6-(4-methoxyphenyl)imidazo[1,2-a]pyrazin-8-amine; 5-(8-fluoroquinolin-6-yl)-6-(1H-pyrazol-5-yl)imidazo[1,2-a]pyrazin-8-amine; 3-[8-amino-5-(quinolin-6-yl)imidazo[1,2-a]pyrazin-6-yl]benzonitrile; 5-[8-amino-6-(5-methylfuran-2-yl)imidazo[1,2-a]pyrazin-5-yl]-1-ethyl-1,2-dihydropyridin-2-one; 6-(5-chloro-6-methoxypyridin-3-yl)-5-(quinolin-6-yl)imidazo[1,2-a]pyrazin-8-amine; 1-{5-[8-amino-5-(quinolin-6-yl)imidazo[1,2-a]pyrazin-6-yl]pyridin-2-yl}ethan-1-one; 6-(3,4-difluorophenyl)-5-(4-methylquinolin-6-yl)imidazo[1,2-a]pyrazin-8-amine; 5-(4-methylquinolin-6-yl)-6-(1,3-thiazol-4-yl)imidazo[1,2-a]pyrazin-8-amine; 8-amino-6-(3-fluorophenyl)-5-(4-methylquinolin-6-yl)imidazo[1,2-a]pyrazine-2-carboxamide; 8-amino-6-(3-fluorophenyl)-N-methyl-5-{3-methylimidazo[1,2-a]pyridin-6-yl}imidazo[1,2-a]pyrazine-2-carboxamide; 8-amino-6-(4-fluorophenyl)-5-(4-methylquinolin-6-yl)imidazo[1,2-a]pyrazine-2-carboxamide; 8-amino-6-(3-fluorophenyl)-5-{3-methylimidazo[1,2-a]pyridin-6-yl}imidazo[1,2-a]pyrazine-2-carboxamide; 8-amino-6-(4-fluorophenyl)-N-methyl-5-(4-methylquinolin-6-yl)imidazo[1,2-a]pyrazine-2-carboxamide; 8-amino-6-(3-fluorophenyl)-5-(1-methyl-1H-1,3-benzodiazol-6-yl)imidazo[1,2-a]pyrazine-2-carboxamide; 8-amino-6-(4-fluorophenyl)-5-(quinolin-6-yl)imidazo[1,2-a]pyrazine-2-carboxamide; ethyl 8-amino-6-(4-fluorophenyl)-5-(4-methylquinolin-6-yl)imidazo[1,2-a]pyrazine-2-carboxylate; ethyl 8-amino-6-(3-cyanophenyl)-5-(8-fluoroquinolin-6-yl)imidazo[1,2-a]pyrazine-2-carboxylate; 6-(4-fluorophenyl)-5-(4-methylquinolin-6-yl)-2-(trifluoromethyl)imidazo[1,2-a]pyrazin-8-amine; 6-(4-fluorophenyl)-5-(quinolin-6-yl)-2-(trifluoromethyl)imidazo[1,2-a]pyrazin-8-amine; 8-amino-6-(4-fluorophenyl)-N-methyl-5-(1-methyl-1H-1,3-benzodiazol-6-yl)imidazo[1,2-a]pyrazine-2-carboxamide; 6-(3-fluorophenyl)-5-(1-methyl-1H-1,3-benzodiazol-6-yl)-2-(morpholine-4-carbonyl)imidazo[1,2-a]pyrazin-8-amine; 6-(3-fluorophenyl)-5-(8-fluoroquinolin-6-yl)-2-[4-(4-methoxybenzoyl)piperazine-1-carbonyl]imidazo[1,2-a]pyrazin-8-amine; 2-[4-(2,4-difluorophenyl)piperazine-1-carbonyl]-6-(3-fluorophenyl)-5-(1-methyl-1H-1,3-benzodiazol-6-yl)imidazo[1,2-a]pyrazin-8-amine; ethyl 8-amino-6-(4-fluorophenyl)-5-(quinolin-6-yl)imidazo[1,2-a]pyrazine-2-carboxylate; 1-[8-amino-6-(4-fluorophenyl)-5-(1-methyl-1H-1,3-benzodiazol-6-yl)imidazo[1,2-a]pyrazine-2-carbonyl]-4-methylpiperidin-4-ol; 8-amino-6-(3-fluorophenyl)-N,N-dimethyl-5-(1-methyl-1H-1,3-benzodiazol-6-yl)imidazo[1,2-a]pyrazine-2-carboxamide; 6-(4-fluorophenyl)-2-(4-methylpiperazine-1-carbonyl)-5-(4-methylquinolin-6-yl)imidazo[1,2-a]pyrazin-8-amine; 8-amino-6-(4-fluorophenyl)-5-{3-methylimidazo[1,2-a]pyridin-6-yl}imidazo[1,2-a]pyrazine-2-carboxamide; 8-amino-6-(4-fluorophenyl)-N-(2-methoxyethyl)-5-(1-methyl-1H-1,3-benzodiazol-6-yl)imidazo[1,2-a]pyrazine-2-carboxamide; 8-amino-6-(4-fluorophenyl)-N,N-dimethyl-5-(4-methylquinolin-6-yl)imidazo[1,2-a]pyrazine-2-carboxamide; 2-[4-(2,4-difluorophenyl)piperazine-1-carbonyl]-6-(4-fluorophenyl)-5-(1-methyl-1H-1,3-benzodiazol-6-yl)imidazo[1,2-a]pyrazin-8-amine; 8-amino-N-({1-[(2,4-difluorophenyl)methyl]piperidin-4-yl}methyl)-6-(4-fluorophenyl)-5-(1-methyl-1H-1,3-benzodiazol-6-yl)imidazo[1,2-a]pyrazine-2-carboxamide; 2-[8-amino-6-(4-fluorophenyl)-5-(1-methyl-1H-1,3-benzodiazol-6-yl)imidazo[1,2-a]pyrazin-2-yl]-1-[4-(2,4-difluorophenyl)piperazin-1-yl]ethan-1-one; 6-(4-fluorophenyl)-5-(4-methylquinolin-6-yl)-2-(piperazine-1-carbonyl)imidazo[1,2-a]pyrazin-8-amine; 6-(4-fluoro-3-methylphenyl)-5-(1-methyl-1H-1,3-benzodiazol-6-yl)imidazo[1,2-a]pyrazin-8-amine; 6-(6-fluoropyridin-3-yl)-5-(1-methyl-1H-1,3-benzodiazol-6-yl)imidazo[1,2-a]pyrazin-8-amine; 6-(4-fluorophenyl)-5-(1-methyl-1H-1,3-benzodiazol-6-yl)-2-(4-methylpiperazine-1-carbonyl)imidazo[1,2-a]pyrazin-8-amine; 6-(6-fluoropyridin-2-yl)-5-(1-methyl-1H-1,3-benzodiazol-6-yl)imidazo[1,2-a]pyrazin-8-amine; 2-[8-amino-6-(4-fluorophenyl)-5-(1-methyl-1H-1,3-benzodiazol-6-yl)imidazo[1,2-a]pyrazin-2-yl]acetamide; [8-amino-6-(4-fluorophenyl)-5-(1-methyl-1H-1,3-benzodiazol-6-yl)imidazo[1,2-a]pyrazin-2-yl]methanol; ethyl 8-amino-6-(4-fluorophenyl)-5-(1-methyl-1H-1,3-benzodiazol-6-yl)imidazo[1,2-a]pyrazine-2-carboxylate; 6-(4-fluorophenyl)-5-(4-methylquinolin-6-yl)-2-(morpholine-4-carbonyl)imidazo[1,2-a]pyrazin-8-amine; 5-(1-methyl-1H-1,3-benzodiazol-6-yl)-6-(pyridin-4-yl)imidazo[1,2-a]pyrazin-8-amine; 5-(1-ethyl-1H-1,3-benzodiazol-6-yl)-6-(4-fluorophenyl)imidazo[1,2-a]pyrazin-8-amine; and 1-[8-amino-6-(4-fluorophenyl)-5-(4-methylquinolin-6-yl)imidazo[1,2-a]pyrazine-2-carbonyl]-4-methylpiperidin-4-ol.

In a second aspect A2, the present invention relates to a pharmaceutical composition comprising a pharmaceutically effective amount of the compound according to formula (I) as defined above in the first aspect A1, and optionally a pharmaceutically acceptable carrier, diluent or excipient. Put in different words, the present invention relates to the compound according to formula (I) as defined above in the first aspect A1, or a pharmaceutical composition comprising a pharmaceutically effective amount of the compound according to formula (I) as defined above in the first aspect A1, for use in medicine.

In a third aspect A3, the present invention relates to a compound according to formula (I) as defined above in the first aspect A1, or a pharmaceutical composition comprising a pharmaceutically effective amount of the compound according to formula (I) as defined above in the first aspect A1, for use in the treatment of a disease selected from the group consisting of cancer, Parkinson's disease, Huntington's disease, Alzheimer's disease, psychosis, stroke, extra pyramidal syndrome (in particular dystonia, akathisia, pseudoparkinsonism and tardive dyskinesia), attention deficit disorder (ADD), attention deficit hyperactivity disorder (ADHD), amyotrophic lateral sclerosis, cirrhosis, fibrosis, fatty liver, addictive behavior, dermal fibrosis (in particular dermal fibrosis in scleroderma), sleep disorders, AIDS, autoimmune diseases, infections, atherosclerosis and ischemia-reperfusion injury. Further indications are described below in the detailed description, together with preferred combinations, namely the compounds of the present invention together with checkpoint inhibitors, wherein such combinations are used for the treatment of cancer.

In a fourth aspect A4, the present invention is concerned with a method for antagonizing the adenosine A2A receptor, wherein said receptor is exposed to at least one compound according to formula (I) as defined above in the first aspect A1, wherein said method is preferably performed outside the human or animal body.

In a fifth aspect A5, the present invention relates to the use of a compound according to formula (I) as defined above in the first aspect A1 as adenosine A2A receptor antagonist.

In the first aspect B1, the present invention relates to a compound of formula (I)

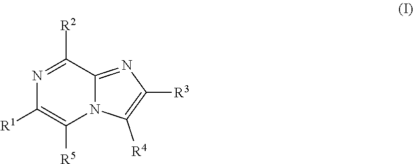

or a salt, stereoisomer, tautomer or N-oxide thereof,
wherein
R¹ is selected from the group consisting of a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said Nand/or S-atoms are independently oxidized or non-oxidized, and wherein each substitutable carbon or heteroatom in the aforementioned cyclic or bicyclic moieties is independently unsubstituted or substituted with one or more, same or different substituents R⁶;
R² is selected from the group consisting of halogen and N(R^{12a})(R^{12b});
R³ is selected from the group consisting of
   (i) H, halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁸;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
R⁴ is H;
R⁵ is selected from the group consisting of a 5- to 9-membered saturated, partially unsaturated or fully unsaturated carbocyclic or heterocyclic ring and a 9- to 12-membered saturated, partially unsaturated or fully unsaturated carbobicyclic or heterobicyclic ring, wherein said heterocyclic or heterobicyclic ring comprises one or more, same or different heteroatoms selected from O, N or S, wherein said Nand/or S-atoms are independently oxidized or non-oxidized, and wherein each substitutable carbon or heteroatom in the aforementioned cyclic or bicyclic moieties is independently unsubstituted or substituted with one or more, same or different substituents R¹⁷;
R⁶ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁷;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
R⁷ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R¹⁰;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
R⁸ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁹;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
R⁹ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R¹⁰;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}) and N(R¹²)S(═O)ₘOR¹²;
R¹⁰ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
R¹¹, R¹², R^{12a}, R^{12b} are independently selected from the group consisting of
   (i) H, C₁-C₆-alkyl, C₂-C₆-alkenyl, and C₂-C₆-alkynyl, wherein each substitutable carbon atom in the aforementioned moieties is independently unsubstituted or substituted with one or more, same or different substituents R¹³;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁴;
R¹³ is selected from the group consisting of
   (i) halogen, CN, NO₂, C₁-C₆-alkyl, C₁—C-haloalkyl, C₂-C₆-alkenyl, C₂-C₆-haloalkenyl, C₂-C₆-alkynyl, C₂-C₆-haloalkynyl, N(R^{15a})(R^{15b}), C(═O)NR^{15a}R^{15b}, S(═O)ₙNR^{15a}R^{15b}, OR¹⁵ and S(═O)ₙR¹⁵;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁴;
R¹⁴ is selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C(═O)R¹⁶, C(═O)OR¹⁵, C(═O)SR¹⁵, C(═O)N(R^{15a})(R^{15b}), OR¹⁵, S(═O)ₙR¹⁵, S(═O)ₙN(R^{15a})(R^{15b}), S(═O)ₘOR¹⁵, N(R^{15a})(R^{15b}), N(R¹⁵)C(═O)R¹⁶, N(R¹⁵)C(═O)OR¹⁵, N(R¹⁵)C(═O)N(R^{15a})(R^{15b}), N(R¹⁵)S(═O)ₙ(R¹⁵), N(R¹⁵)S(═O)ₘN(R^{15a})(R^{15b}), and N(R¹⁵)S(═O)ₘOR¹⁵;
R¹⁵, R^{15a}, R^{15b}, R¹⁶ are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
R¹⁷ is selected from the group consisting of
   (i) halogen, CN, NO₂, C₁-C₄-alkyl, C₂-C₄-alkenyl, and C₂-C₄-alkynyl, wherein each substitutable carbon or hetero-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
R¹⁸ is selected from the group consisting of halogen, N(R^{20a})(R^{20b}), and OR²⁰;
R¹⁹, R²⁰, R^{20a}, R^{20b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
and wherein
n is 0, 1 or 2; and
m is 1 or 2.

In a preferred embodiment, R² is NH₂, wherein all other substituents have the meaning as defined above in the first aspect B1.

In another preferred embodiment, R⁵ is selected from the group consisting of a 5- to 6-membered saturated, partially unsaturated or fully unsaturated carbocyclic or heterocyclic ring and a 9- to 10-membered saturated, partially unsaturated or fully unsaturated carbobicyclic or heterobicyclic ring, wherein said heterocyclic or heterobicyclic ring comprises one or more N-atoms, and wherein said N-atoms are independently oxidized or non-oxidized, and wherein each substitutable carbon or heteroatom in the aforementioned cyclic moieties is independently substituted with one or more, same or different substituents R¹⁷, and wherein each substitutable carbon or heteroatom in the aforementioned bicyclic moieties is independently unsubstituted or substituted with one or more, same or different substituents R¹⁷ and wherein all other substituents have the meaning as defined above in the first aspect B1.

In another preferred embodiment, R⁵ is selected from the group consisting of a 5- to 6-membered fully unsaturated carbocyclic or heterocyclic ring and a 9- to 10-membered fully unsaturated carbobicyclic or heterobicyclic ring, wherein said heterocyclic or heterobicyclic ring comprises one or more N-atoms, and wherein said N-atoms are independently oxidized or non-oxidized, and wherein each substitutable carbon or heteroatom in the aforementioned cyclic moieties is independently substituted with one or more, same or different substituents R¹⁷, and wherein each substitutable carbon or heteroatom in the aforementioned bicyclic moieties is independently unsubstituted or substituted with one or more, same or different substituents R¹⁷ and wherein all other substituents have the meaning as defined above in the first aspect B1.

In yet another preferred embodiment R⁵ has the formula (S1)

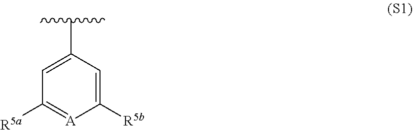

and wherein
   A is N or CR^{5c};
R^{5a}, R^{5b}, R^{5c} are independently selected from the group consisting of
   (i) H, halogen, CN, NO₂, C₁-C₄-alkyl, C₂-C₄-alkenyl, and C₂-C₄-alkynyl, wherein each substitutable carbon or hetero-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
      with the proviso that at least one of R^{5a}, R^{5b}, R^{5c} is not H;
or
R^{5a} is selected from the group consisting of
   (i) H, halogen, CN, NO₂, C₁-C₄-alkyl, C₂-C₄-alkenyl, and C₂-C₄-alkynyl, wherein each substitutable carbon or hetero-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
      and
R^{5b} and R^{5c} together with the atoms to which they are attached form a 5- to 6-membered fully unsaturated carbocyclic or heterocyclic ring, wherein said heterocyclic ring comprises one or more N-atoms, and wherein said N-atoms are independently oxidized or non-oxidized, and wherein each substitutable carbon or heteroatom in the aforementioned cyclic moieties is independently unsubstituted or substituted with one or more, same or different substituents selected from the group consisting of
   (i) H, halogen, CN, NO₂, C₁-C₄-alkyl, C₂-C₄-alkenyl, and C₂-C₄-alkynyl, wherein each substitutable carbon or hetero-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
   and wherein all other substituents have the meaning as defined above.

In still another preferred embodiment, R⁵ is selected from the group consisting of a 6-membered fully unsaturated carbocyclic or heterocyclic ring and a 9- to 10-membered fully unsaturated heterobicyclic ring, wherein said heterocyclic or heterobicyclic ring comprises one or more N-atoms, and wherein said N-atoms are independently oxidized or non-oxidized, and wherein each substitutable carbon or heteroatom in the aforementioned cyclic moieties is independently substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, OR²⁰, and N(R^{20a})(R^{20b}), and wherein each substitutable carbon or heteroatom in the aforementioned bicyclic moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, OR²⁰, and N(R^{20a})(R^{20b}); and wherein all other substituents have the meaning as defined above in the first aspect B1.

In yet another preferred embodiment, R⁵ is selected from the group consisting of a 6-membered fully unsaturated carbocyclic or heterocyclic ring and a 9- to 10-membered fully unsaturated heterobicyclic ring, wherein said heterocyclic or heterobicyclic ring comprises one or more N-atoms, and wherein said N-atoms are independently oxidized or non-oxidized, and wherein each substitutable carbon or heteroatom in the aforementioned cyclic moieties is independently substituted with one or more, same or different substituents selected from the group consisting of halogen, —OH, —OCH₃, —CH₃, —CF₃, and —NHCH₃, and wherein each substitutable carbon or heteroatom in the aforementioned bicyclic moieties is independently unsubstituted or substituted with one or more, same or different substituents selected from the group consisting of halogen, —OH, —OCH₃, —CH₃, —CF₃, and —NHCH₃; and wherein all other substituents have the meaning as defined above in the first aspect B1.

In another preferred embodiment, R⁶ is selected from the group consisting of
(i) halogen, CN, NO₂, C₁-C₆-alkyl, C₂-C₆-alkenyl, and C₂-C₆-alkynyl, wherein each substitutable carbon or hetero-atom in the aforementioned moieties is unsubstituted or substituted with one or more, same or different substituents R⁷;
(ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²; and
wherein R⁷ is selected from the group consisting of
(i) halogen, CN, NO₂, C₁-C₆-alkyl, C₂-C₆-alkenyl, and C₂-C₆-alkynyl,
(ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹².

In another preferred embodiment, R⁸ is selected from the group consisting of
(i) halogen, CN, NO₂, C₁-C₆-alkyl, C₂-C₆-alkenyl, and C₂-C₆-alkynyl, wherein each substitutable carbon or hetero-atom in the aforementioned moieties is unsubstituted or substituted with one or more, same or different substituents R⁹;
(ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²; and
wherein R⁹ is selected from the group consisting of
(i) halogen, CN, NO₂, C₁-C₆-alkyl, C₂-C₆-alkenyl, and C₂-C₆-alkynyl,
(ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹².

In another preferred embodiment, R¹ is selected from the group consisting of a 5- to 6-membered fully unsaturated carbocyclic or heterocyclic ring, wherein said heterocyclic ring comprises one or more, same or different heteroatoms selected from O, N or S, wherein said Nand/or S-atoms are independently oxidized or non-oxidized, and wherein each substitutable carbon or heteroatom in the aforementioned cyclic moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl; and wherein all other substituents have the meaning as defined above in the first aspect B1.

In another preferred embodiment, R¹ is selected from the group consisting of a 5- to 6-membered fully unsaturated carbocyclic or heterocyclic ring, wherein said heterocyclic or ring comprises one or more, same or different heteroatoms selected from O, N or S, wherein said Nand/or S-atoms are independently oxidized or non-oxidized, and wherein each substitutable carbon or heteroatom in the aforementioned cyclic moieties is independently unsubstituted or substituted with one or more, same or different substituents selected from the group consisting of halogen, CN, and trifluoromethyl; and wherein all other substituents have the meaning as above in the first aspect B1.

In yet another preferred embodiment, R³ is selected from the group consisting of
(i) H, halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
(ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
and wherein all other substituents have the meaning as defined above in the first aspect B1.

In still another preferred embodiment, R³ is selected from the group consisting of
(i) H and a 6-membered saturated, partially unsaturated or fully unsaturated carbocyclic ring, wherein each substitutable carbonatom in the aforementioned moieties is independently unsubstituted or substituted with one or more, same or different substituents selected from the group consisting of CN and NO₂;
(ii) C(═O)NH₂;
and wherein all other substituents have the meaning as defined above in the first aspect B1.

In another preferred embodiment, R¹¹, R¹², R^{12a}, R^{12b} are independently selected from the group consisting of H, C₁—C-alkyl, C₂-C₃-alkenyl and C₂-C₃-alkynyl.

In another embodiment, said compound is selected from the group consisting of 4-{8-amino-6-phenylimidazo[1,2-a]pyrazin-5-yl}-2-chlorophenol; 4-[8-amino-6-(furan-2-yl)imidazo[1,2-a]pyrazin-5-yl]-2-chlorophenol; 5-(2,6-dimethylpyridin-4-yl)-6-phenylimidazo[1,2-a]pyrazin-8-amine; 5-[2-methyl-6-(trifluoromethyl)pyridin-4-yl]-6-phenylimidazo[1,2-a]pyrazin-8-amine; 4-[8-bromo-6-(furan-2-yl)imidazo[1,2-a]pyrazin-5-yl]-2-chlorophenol; 4-[8-amino-6-(4-fluorophenyl)imidazo[1,2-a]pyrazin-5-yl]-2-chlorophenol; 6-(4-fluorophenyl)-5-[2-methyl-6-(trifluoromethyl)pyridin-4-yl]imidazo[1,2-a]pyrazin-8-amine; 5-(2,6-dimethylpyridin-4-yl)-6-(4-fluorophenyl)imidazo[1,2-a]pyrazin-8-amine; 6-(3-fluorophenyl)-5-[2-methyl-6-(trifluoromethyl)pyridin-4-yl]imidazo[1,2-a]pyrazin-8-amine; 3-{8-amino-5-[2-methyl-6-(trifluoromethyl)pyridin-4-yl]imidazo[1,2-a]pyrazin-6-yl}benzonitrile; 3-[8-amino-5-(3-chloro-4-hydroxyphenyl)imidazo[1,2-a]pyrazin-6-yl]benzonitrile; 4-[8-amino-6-(3-fluorophenyl)imidazo[1,2-a]pyrazin-5-yl]-2-chlorophenol; 4-[8-amino-2-(3-nitrophenyl)-6-phenylimidazo[1,2-a]pyrazin-5-yl]-2-chlorophenol; 5-(1H-indazol-5-yl)-6-phenylimidazo[1,2-a]pyrazin-8-amine; 4-{8-amino-6-phenylimidazo[1,2-a]pyrazin-5-yl}-2,6-dichlorophenol; 4-{8-amino-2-cyclohexyl-6-phenylimidazo[1,2-a]pyrazin-5-yl}-2-chlorophenol; 4-{8-amino-6-phenylimidazo[1,2-a]pyrazin-5-yl}-2-bromo-6-chlorophenol; 4-{8-amino-6-[4-(trifluoromethyl)phenyl]imidazo[1,2-a]pyrazin-5-yl}-2-chlorophenol; 3-[8-amino-5-(3-chloro-4-hydroxyphenyl)-6-phenylimidazo[1,2-a]pyrazin-2-yl]benzonitrile; 4-[8-amino-5-(3-chloro-4-hydroxyphenyl)imidazo[1,2-a]pyrazin-6-yl]benzonitrile; 4-{8-amino-6-phenylimidazo[1,2-a]pyrazin-5-yl}-N-methylpyridin-2-amine; 4-{8-amino-2,6-diphenylimidazo[1,2-a]pyrazin-5-yl}-2-chlorophenol; 4-[8-amino-6-(3-fluorophenyl)imidazo[1,2-a]pyrazin-5-yl]phenol; 4-[8-amino-6-(3-fluorophenyl)imidazo[1,2-a]pyrazin-5-yl]-N-methylpyridin-2-amine; 8-amino-5-(3-chloro-4-hydroxyphenyl)-6-phenylimidazo[1,2-a]pyrazine-2-carboxamide; 4-[8-amino-6-(4-fluorophenyl)imidazo[1,2-a]pyrazin-5-yl]-N-methylpyridin-2-amine; and 6-(3-fluorophenyl)-5-(quinolin-6-yl)imidazo[1,2-a]pyrazin-8-amine.

In a second aspect B2, the present invention relates to a pharmaceutical composition comprising a pharmaceutically effective amount of the compound according to formula (I) as defined above in the first aspect B1, and optionally a pharmaceutically acceptable carrier, diluent or excipient. Put in different words, the present invention relates to the compound according to formula (I) as defined above, or a pharmaceutical composition comprising a pharmaceutically effective amount of the compound according to formula (I) as defined above in the first aspect B1, for use in medicine.

In a third aspect B3, the present invention relates to a compound according to formula (I) as defined above in the first aspect B1, or a pharmaceutical composition comprising a pharmaceutically effective amount of the compound according to formula (I) as defined above in the first aspect B1, for use in the treatment of a disease selected from the group consisting of cancer, Parkinson's disease, Huntington's disease, Alzheimer's disease, psychosis, stroke, extra pyramidal syndrome (in particular dystonia, akathisia, pseudoparkinsonism and tardive dyskinesia), attention deficit disorder (ADD), attention deficit hyperactivity disorder (ADHD), amyotrophic lateral sclerosis, cirrhosis, fibrosis, fatty liver, addictive behavior, dermal fibrosis (in particular dermal fibrosis in scleroderma), sleep disorders, AIDS, autoimmune diseases, infections, atherosclerosis and ischemia-reperfusion injury. Further indications are described below in the detailed description, together with preferred combinations, namely the compounds of the present invention together with checkpoint inhibitors, wherein such combinations are used for the treatment of cancer.

In a fourth aspect B4, the present invention is concerned with a method for antagonizing the adenosine A2A receptor, wherein said receptor is exposed to at least one compound according to formula (I) as defined above in the first aspect B1, wherein said method is preferably performed outside the human or animal body.

In a fifth aspect B5, the present invention relates to the use of a compound according to formula (I) as defined above in the first aspect B1 as adenosine A2A receptor antagonist.

### Detailed Description Relating to Aspects A1 to A5

In the following, preferred embodiments of the substituents in the above formula (I) are described in further detail.

The following embodiments relate to R¹ as defined above in the first aspect A1.

In embodiment 1(A), R¹ is selected from the group consisting of a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁶;

wherein R⁶ is selected from the group consisting of
(i) halogen, CN, NO₂, C₁-C₆-alkyl, C₂-C₆-alkenyl, and C₂-C₆-alkynyl, wherein each substitutable carbon-atom in the aforementioned moieties is unsubstituted or substituted with one or more, same or different substituents R⁷;
(ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
and/or two R⁶ present on one C-atom together form ═O; and
wherein R⁷ is selected from the group consisting of
(i) halogen, CN, NO₂, C₁-C₆-alkyl, C₂-C₆-alkenyl, and C₂-C₆-alkynyl,
(ii) C(═O)R²¹, C(═O)OR²², C(═O)SR²², C(═O)N(R^{22a})(R^{22b}), OR²², S(═O)ₙR²², S(═O)ₙN(R^{22a})(R^{22b}), S(═O)ₘOR²², N(R^{22a})(R^{22b}), N(R²²)C(═O)R²¹, N(R²²)C(═O)OR²², N(R²²)C(═O)N(R^{22a})(R^{22b}), N(R²²)S(═O)ₙ(R²²), N(R²²)S(═O)ₘN(R^{22a})(R^{22b}), and N(R²²)S(═O)ₘOR²²;
   The following substituent meanings are relevant in connection with embodiment 1(A):
R¹¹, R¹², R^{12a}, R^{12b} are independently selected from the group consisting of
   (i) H, C₁-C₆-alkyl, C₂-C₆-alkenyl, and C₂-C₆-alkynyl, wherein each substitutable carbon atom in the aforementioned moieties is independently unsubstituted or substituted with one or more, same or different substituents R¹³;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁴;
R¹³ is selected from the group consisting of
   (i) halogen, CN, NO₂, C₁-C₆-alkyl, C₁—C-haloalkyl, C₂-C₆-alkenyl, C₂-C₆-haloalkenyl, C₂-C₆-alkynyl, C₂-C₆-haloalkynyl, N(R^{15a})(R^{15b}), C(═O)NR^{15a}R^{15b}, S(═O)ₙNR^{15a}R^{15b}, OR¹⁵ and S(═O)ₙR¹⁵;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁴;
R¹⁴ is selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C(═O)R¹⁶, C(═O)OR¹⁵, C(═O)SR¹⁵, C(═O)N(R^{15a})(R^{15b}), OR¹⁵, S(═O)ₙR¹⁵, S(═O)ₙN(R^{15a})(R^{15b}), S(═O)ₘOR¹⁵, N(R^{15a})(R^{15b}), N(R¹⁵)C(═O)R¹⁶, N(R¹⁵)C(═O)OR¹⁵, N(R¹⁵)C(═O)N(R^{15a})(R^{15b}), N(R¹⁵)S(═O)ₙ(R¹⁵), N(R¹⁵)S(═O)ₘN(R^{15a})(R^{15b}), and N(R¹⁵)S(═O)ₘOR¹⁵;
R¹⁵, R^{15a}, R^{15b}, R¹⁶ are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
R²¹, R²², R^{22a}, R^{22b} are independently selected from the group consisting of
   (i) H, C₁-C₆-alkyl, C₂-C₆-alkenyl, and C₂-C₆-alkynyl, wherein each substitutable carbon atom in the aforementioned moieties is independently unsubstituted or substituted with one or more, same or different substituents R¹³;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁴;
and wherein
n is 0, 1 or 2; and
m is 1 or 2.

Preferably, the following substituent meanings are relevant in connection with embodiment 1(A):
R¹¹, R¹², R^{12a}, R^{12b}, R²¹, R²², R^{22a}, R^{22b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl.

In a preferred embodiment 1(B), R¹ is selected from the group consisting of a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁶;
wherein R⁶ is selected from the group consisting of
   (i) halogen, CN, NO₂, C₁-C₆-alkyl, C₂-C₆-alkenyl, and C₂-C₆-alkynyl, wherein each substitutable carbon-atom in the aforementioned moieties is unsubstituted or substituted with one or more, same or different substituents R⁷;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
   and/or two R⁶ present on one C-atom together form ═O;
   wherein R⁷ is selected from the group consisting of
   (i) halogen, CN, NO₂, C₁-C₆-alkyl, C₂-C₆-alkenyl, and C₂-C₆-alkynyl,
   (ii) C(═O)R²¹, C(═O)OR²², C(═O)SR²², C(═O)N(R^{22a})(R^{22b}), OR²², S(═O)ₙR²², S(═O)ₙN(R^{22a})(R^{22b}), S(═O)ₘOR²², N(R^{22a})(R^{22b}), N(R²²)C(═O)R²¹, N(R²²)C(═O)OR²², N(R²²)C(═O)N(R^{22a})(R^{22b}), N(R²²)S(═O)ₙ(R²²), N(R²²)S(═O)ₘN(R^{22a})(R^{22b}), and N(R²²)S(═O)ₘOR²²;
   wherein R¹¹, R¹², R^{12a}, R^{12b}, R²¹, R²², R^{22a}, R^{22b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
   and wherein
   n is 0, 1 or 2; and
   m is 1 or 2.

In another preferred embodiment 1(C), R1 is selected from the group consisting of a 5- to 6-membered fully unsaturated carbocyclic or heterocyclic ring and a 9- to 10-membered fully unsaturated carbobicyclic or heterobicyclic ring, wherein said heterocyclic or heterobicyclic ring comprises one or more, same or different heteroatoms selected from O, N or S, wherein said Nand/or S-atoms are independently oxidized or non-oxidized, and wherein each substitutable carbon or heteroatom in the aforementioned cyclic or bicyclic moieties is independently unsubstituted or substituted with one or more, same or different substituents R⁶;
wherein R⁶ is selected from the group consisting of
   (i) halogen, CN, NO₂, C₁-C₃-alkyl, C₂-C₃-alkenyl, and C₂-C₃-alkynyl, wherein each substitutable carbon-atom in the aforementioned moieties is unsubstituted or substituted with one or more, same or different substituents R⁷;
   (ii) C(═O)R¹¹, C(═O)OR¹², C(═O)N(R^{12a})(R^{12b}), OR¹², N(R^{12a})(R^{12b}), N(R¹²)C(═O)R¹¹, N(R¹²)C(═O)OR¹², and N(R¹²)C(═O)N(R^{12a})(R^{12b});
   and/or two R⁶ present on one C-atom together form ═O;
   wherein R⁷ is selected from the group consisting of
   (i) halogen, CN, NO₂, C₁-C₆-alkyl, C₂-C₆-alkenyl, and C₂-C₆-alkynyl,
   (ii) C(═O)R²¹, C(═O)OR²², C(═O)N(R^{22a})(R^{22b}), OR²², N(R^{22a})(R^{22b}), N(R²²)C(═O)R²¹, N(R²²)C(═O)OR²², and N(R²²)C(═O)N(R^{22a})(R^{22b});
   wherein R¹¹, R¹², R^{12a}, R^{12b}, R²¹, R²², R^{22a}, R^{22b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl.

In another embodiment 1(D), R¹ is a 5- to 6-membered fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, OH, O(C₁-C₄-alkyl), NH₂, NH(C₁-C₄-alkyl), and N(C₁-C₄-alkyl)(C₁-C₄-alkyl).

In another embodiment 1(E), R¹ is a 5- to 6-membered fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is independently unsubstituted or substituted with one or more, same or different substituents selected from the group consisting of halogen, CN, CF₃, CH₃, OH, OCH₃, NH₂, NH(CH₃), and N(CH₃)(CH₃).

The following embodiments relate to R³ as defined above in the first aspect A1.
In embodiment 3(A), R³ is selected from the group consisting of
   (i) H, halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁸;
   (ii) C(═O)R²⁵, C(═O)OR²⁶, C(═O)SR²⁶, C(═O)N(R^{26a})(R^{26b}), OR²⁶, S(═O)ₙR²⁶, S(═O)ₙN(R^{26a})(R^{26b}), S(═O)ₘOR²⁶, N(R^{26a})(R^{26b}), N(R²⁶)C(═O)R²⁵, N(R²⁶)C(═O)OR²⁶, N(R²⁶)C(═O)N(R^{26a})(R^{26b}), N(R²⁶)S(═O)ₙ(R²⁶), N(R²⁶)S(═O)ₘN(R^{26a})(R^{26b}), and N(R²⁶)S(═O)ₘOR²⁶;
wherein R⁸ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁹;
   (ii) C(═O)R²⁷, C(═O)OR²⁸, C(═O)SR²⁸, C(═O)N(R^{28a})(R^{28b}), OR²⁸, S(═O)ₙR²⁸, S(═O)ₙN(R^{28a})(R^{28b}), S(═O)ₘOR²⁸, N(R^{28a})(R^{28b}), N(R²⁸)C(═O)R²⁷, N(R²⁸)C(═O)OR²⁸, N(R²⁸)C(═O)N(R^{28a})(R^{28b}), N(R²⁸)S(═O)ₙ(R²⁸), N(R²⁸)S(═O)ₘN(R^{28a})(R^{28b}), and N(R²⁸)S(═O)ₘOR²⁸;
wherein R⁹ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R²⁹;
   (ii) C(═O)R³⁰, C(═O)OR³¹, C(═O)SR³¹, C(═O)N(R^{31a})(R^{31b}), OR³¹, S(═O)ₙR³¹, S(═O)ₙN(R^{31a})(R^{31b}), S(═O)ₘOR³¹, N(R^{31a})(R^{31b}), N(R³¹)C(═O)R³⁰, N(R³¹)C(═O)OR³¹, N(R³¹)C(═O)N(R^{31a})(R^{31b}), N(R³¹)S(═O)ₙ(R³¹), N(R³¹)S(═O)ₘN(R^{31a})(R^{31b}), and N(R³¹)S(═O)ₘOR³¹;
wherein R²⁵, R²⁶, R^{26a}, R^{26b} are independently selected from the group consisting of H, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R³²;
wherein R²⁷, R²⁸, R^{28a}, R^{28b} are independently selected from the group consisting of H, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R³³;
wherein R²⁹ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(═O)R³⁴, C(═O)OR³⁵, C(═O)SR³⁵, C(═O)N(R^{35a})(R^{35b}), OR³⁵, S(═O)ₙR³⁵, S(═O)ₙN(R^{35a})(R^{35b}), S(═O)ₘOR³⁵, N(R^{35a})(R^{35b}), N(R³⁵)C(═O)R³⁴, N(R³⁵)C(═O)OR³⁵, N(R³⁵)C(═O)N(R^{35a})(R^{35b}), N(R³⁵)S(═O)ₙ(R³⁵), N(R³⁵)S(═O)ₘN(R^{35a})(R^{35b}), and N(R³⁵)S(═O)ₘOR³⁵;
wherein R³⁰, R³¹, R^{31a}, R^{31b} are independently selected from the group consisting of H, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R³⁷;
wherein R³² is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R³⁸;
   (ii) C(═O)R³⁹, C(═O)OR⁴⁰, C(═O)SR⁴⁰, C(═O)N(R^{40a})(R^{40b}), OR⁴⁰, S(═O)ₙR⁴⁰, S(═O)ₙN(R^{40a})(R^{40b}), S(═O)ₘOR⁴⁰, N(R^{40a})(R^{40b}), N(R⁴⁰)C(═O)R³⁹, N(R⁴⁰)C(═O)OR⁴⁰, N(R⁴⁰)C(═O)N(R^{40a})(R^{40b}), N(R⁴⁰)S(═O)ₙ(R⁴⁰), N(R⁴⁰)S(═O)ₘN(R^{40a})(R^{40b}), and N(R⁴⁰)S(═O)ₘOR⁴⁰;
wherein R³³ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁴¹;
   (ii) C(═O)R⁴², C(═O)OR⁴³, C(═O)SR⁴³, C(═O)N(R^{43a})(R^{43b}), OR⁴³, S(═O)ₙR⁴³, S(═O)ₙN(R^{43a})(R^{43b}), S(═O)ₘOR⁴³, N(R^{43a})(R^{43b}), N(R⁴³)C(═O)R⁴², N(R⁴³)C(═O)OR⁴³, N(R⁴³)C(═O)N(R^{43a})(R^{43b}), N(R⁴³)S(═O)ₙ(R⁴³), N(R⁴³)S(═O)ₘN(R^{43a})(R^{43b}), and N(R⁴³)S(═O)ₘOR⁴³;
wherein R³⁴, R³⁵, R^{35a}, R^{35b} are independently selected from the group consisting of
   (i) H, C₁-C₆-alkyl, C₂-C₆-alkenyl, and C₂-C₆-alkynyl, wherein each substitutable carbon atom in the aforementioned moieties is independently unsubstituted or substituted with one or more, same or different substituents R³⁶;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁵²;
wherein R³⁶ is selected from the group consisting of
   (i) halogen, CN, NO₂, C₁-C₆-alkyl, C₁—C-haloalkyl, C₂-C₆-alkenyl, C₂-C₆-haloalkenyl, C₂-C₆-alkynyl, C₂—C-haloalkynyl, N(R^{53a})(R^{53b}), C(═O)NR^{53a}R^{53b}, S(═O)ₙNR^{53a}R^{53b}, OR⁵³ and S(═O)ₙR⁵³;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁵²;
wherein R³⁷ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, OH, O(C₁-C₄-alkyl), NH₂, NH(C₁-C₄-alkyl), and N(C₁-C₄-alkyl)(C₁-C₄-alkyl);
wherein R³⁸ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁴⁴;
   (ii) C(═O)R⁴⁵, C(═O)OR⁴⁶, C(═O)SR⁴⁶, C(═O)N(R^{4a})(R^{46b}), OR⁴⁶, S(═O)ₙR⁴⁶, S(═O)ₙN(R^{46a})(R^{46b}), S(═O)ₘOR⁴⁶, N(R^{46a})(R^{46b}), N(R⁴⁶)C(═O)R⁴⁵, N(R⁴⁶)C(═O)OR⁴⁶, N(R⁴⁶)C(═O)N(R^{46a})(R^{46b}), N(R⁴⁶)S(═O)ₙ(R⁴⁶), N(R⁴⁶)S(═O)ₘN(R^{46a})(R^{46b}), and N(R⁴⁶)S(═O)ₘOR⁴⁶;
wherein R³⁹, R⁴⁰, R^{40a}, R^{40b} are independently selected from the group consisting of H, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁴⁷;
wherein R⁴¹ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, OH, O(C₁-C₄-alkyl), NH₂, NH(C₁-C₄-alkyl), and N(C₁-C₄-alkyl)(C₁-C₄-alkyl);
wherein R⁴², R⁴³, R^{43a}, R^{43b} are independently selected from the group consisting of H, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁴⁸;
wherein R⁴⁴ is selected from the group consisting of halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁴⁹;
wherein R⁴⁵, R⁴⁶, R^{46a}, R^{46b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
wherein R⁴⁷ is selected from the group consisting of halogen, CN, NO₂, OH, O(C₁-C₄-alkyl), NH₂, NH(C₁-C₄-alkyl), and N(C₁-C₄-alkyl)(C₁-C₄-alkyl), C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁵⁰;
wherein R⁴⁸ is selected from the group consisting of halogen, CN, NO₂, OH, O(C₁-C₄-alkyl), NH₂, NH(C₁-C₄-alkyl), and N(C₁-C₄-alkyl)(C₁-C₄-alkyl), C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁵¹;
wherein R⁴⁹, R⁵⁰, R⁵¹ are independently selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, OH, O(C₁-C₄-alkyl), NH₂, NH(C₁-C₄-alkyl), and N(C₁-C₄-alkyl)(C₁-C₄-alkyl);
wherein R⁵² is selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C(═O)R⁵⁴, C(═O)OR⁵³, C(═O)SR⁵³, C(═O)N(R^{53a})(R^{53b}), OR⁵³, S(═O)ₙR⁵³, S(═O)ₙN(R^{53a})(R^{53b}), S(═O)ₘOR⁵³, N(R^{53a})(R^{53b}), N(R⁵³)C(═O)R⁵⁴, N(R⁵³)C(═O)OR⁵³, N(R⁵³)C(═O)N(R^{53a})(R^{53b}), N(R⁵³)S(═O)ₙ(R⁵³), N(R⁵³)S(═O)ₘN(R^{53a})(R^{53b}), and N(R⁵³)S(═O)ₘOR⁵³;
wherein R⁵³, R^{53a}, R^{53b}, R⁵⁴ are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
and wherein
n is 0, 1 or 2; and
m is 1 or 2.
In a preferred embodiment 3(B), R³ is selected from the group consisting of
   (i) H, C₁-C₆-alkyl, a 3- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁸;
   (ii) C(═O)R²⁵, C(═O)OR²⁶, C(═O)N(R^{26a})(R^{26b}), OR²⁶, N(R^{26a})(R^{26b}), N(R²⁶)C(═O)R²⁵, N(R²⁶)C(═O)OR²⁶, and N(R²⁶)C(═O)N(R^{26a})(R^{26b});
wherein R⁸ is selected from the group consisting of
   (i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁹;
   (ii) C(═O)R²⁷, C(═O)OR²⁸, C(═O)N(R^{28a})(R^{28b}), OR²⁸, N(R^{28a})(R^{28b}), N(R²⁸)C(═O)R²⁷, N(R²⁸)C(═O)OR²⁸, and N(R²⁸)C(═O)N(R^{28a})(R^{28b});
wherein R⁹ is selected from the group consisting of
   (i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R²⁹;
   (ii) C(═O)R³⁰, C(═O)OR³¹, C(═O)N(R^{31a})(R^{31b}), OR³¹, N(R^{31a})(R^{31b}), N(R³¹)C(═O)R³⁰, N(R³¹)C(═O)OR³¹, N(R³¹)C(═O)N(R^{31a})(R^{31b});
wherein R²⁵, R²⁶, R^{26a}, R^{26b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³²;
wherein R²⁷, R²⁸, R^{28a}, R^{28b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³³;
wherein R²⁹ is selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein R³⁰, R³¹, R^{31a}, R^{31b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³⁷;
wherein R³² is selected from the group consisting of
   (i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³⁸;
   (ii) C(═O)R³⁹, C(═O)OR⁴⁰, C(═O)N(R^{40a})(R^{40b}), OR⁴⁰, N(R^{40a})(R^{40b}), N(R⁴⁰)C(═O)R³⁹, N(R⁴⁰)C(═O)OR⁴⁰, N(R⁴⁰)C(═O)N(R^{40a})(R^{40b});
wherein R³³ is selected from the group consisting of
   (i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴¹;
   (ii) C(═O)R⁴², C(═O)OR⁴³, C(═O)N(R^{43a})(R^{43b}), OR⁴³, N(R^{43a})(R^{43b}), N(R⁴³)C(═O)R⁴², N(R⁴³)C(═O)OR⁴³, N(R⁴³)C(═O)N(R^{43a})(R^{43b});
wherein R³⁷ is selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein R³⁸ is selected from the group consisting of
   (i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁴;
   (ii) C(═O)R⁴⁵, C(═O)OR⁴⁶, C(═O)N(R^{46a})(R^{46b}), OR⁴⁶, N(R^{46a})(R^{46b}), N(R⁴⁶)C(═O)R⁴⁵, N(R⁴⁶)C(═O)OR⁴⁶, N(R⁴⁶)C(═O)N(R^{46a})(R^{46b});
wherein R³⁹, R⁴⁰, R^{40a}, R^{40b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁷;
wherein R⁴¹ is selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein R⁴², R⁴³, R^{43a}, R^{43b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁸;
wherein R⁴⁴ is selected from the group consisting of halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁹;
wherein R⁴⁵, R⁴⁶, R^{46a}, R^{46b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
wherein R⁴⁷ is selected from the group consisting of halogen, CN, NO₂, OH, O(C₁-C₄-alkyl), NH₂, NH(C₁-C₄-alkyl), and N(C₁-C₄-alkyl)(C₁-C₄-alkyl), C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁵⁰;
wherein R⁴⁸ is selected from the group consisting of halogen, CN, NO₂, OH, O(C₁-C₄-alkyl), NH₂, NH(C₁-C₄-alkyl), and N(C₁-C₄-alkyl)(C₁-C₄-alkyl), C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁵¹;
wherein R⁴⁹, R⁵⁰, R⁵¹ are independently selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl).
In further embodiment 3(C), R³ is selected from the group consisting of
   (i) H, C₁-C₆-alkyl, a 3- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁸;
   (ii) C(═O)R²⁵, C(═O)OR²⁶, C(═O)N(R^{26a})(R^{26b}), OR²⁶, N(R^{26a})(R^{26b}), N(R²⁶)C(═O)R²⁵, N(R²⁶)C(═O)OR²⁶, and N(R²⁶)C(═O)N(R^{26a})(R^{26b});
wherein R⁸ is selected from the group consisting of
   (i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁹;
   (ii) C(═O)R²⁷, C(═O)OR²⁸, C(═O)N(R^{28a})(R^{28b}), OR²⁸, N(R^{28a})(R^{28b}), N(R²⁸)C(═O)R²⁷, N(R²⁸)C(═O)OR²⁸, and N(R²⁸)C(═O)N(R^{28a})(R^{28b});
wherein R⁹ is selected from the group consisting of
   (i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R²⁹;
   (ii) C(═O)R³⁰, C(═O)OR³¹, C(═O)N(R^{31a})(R^{31b}), OR³¹, N(R^{31a})(R^{31b}), N(R³¹)C(═O)R³⁰, N(R³¹)C(═O)OR³¹, N(R³¹)C(═O)N(R^{31a})(R^{31b});
wherein R²⁵, R²⁶, R^{26a}, R^{26b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³²;
wherein R²⁷, R²⁸, R^{28a}, R^{28b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³³;
wherein R²⁹ is selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein R³⁰, R³¹, R^{31a}, R^{31b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³⁷;
wherein R³² is selected from the group consisting of
   (i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³⁸;
   (ii) C(═O)R³⁹, C(═O)OR⁴⁰, C(═O)N(R^{40a})(R^{40b}), OR⁴⁰, N(R^{40a})(R^{40b}), N(R⁴⁰)C(═O)R³⁹, N(R⁴⁰)C(═O)OR⁴⁰, N(R⁴⁰)C(═O)N(R^{40a})(R^{40b});
wherein R³³ is selected from the group consisting of
   (i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴¹;
   (ii) C(═O)R⁴², C(═O)OR⁴³, C(═O)N(R^{43a})(R^{43b}), OR⁴³, N(R^{43a})(R^{43b}), N(R⁴³)C(═O)R⁴², N(R⁴³)C(═O)OR⁴³, N(R⁴³)C(═O)N(R^{43a})(R^{43b});
wherein R³⁷ is selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein R³⁸ is selected from the group consisting of
   (i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁴;
   (ii) C(═O)R⁴⁵, C(═O)OR⁴⁶, C(═O)N(R^{46a})(R^{46b}), OR⁴⁶, N(R^{46a})(R^{46b}), N(R⁴⁶)C(═O)R⁴⁵, N(R⁴⁶)C(═O)OR⁴⁶, N(R⁴⁶)C(═O)N(R^{46a})(R^{46b});
wherein R³⁹, R⁴⁰, R^{40a}, R^{40b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁷;
wherein R⁴¹ is selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein R⁴², R⁴³, R^{43a}, R^{43b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁸;
wherein R⁴⁴ is selected from the group consisting of halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁹;
wherein R⁴⁵, R⁴⁶, R^{46a}, R^{46b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
wherein R⁴⁷ is selected from the group consisting of halogen, CN, NO₂, OH, O(C₁-C₄-alkyl), NH₂, NH(C₁-C₄-alkyl), and N(C₁-C₄-alkyl)(C₁-C₄-alkyl), C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁵⁰;
wherein R⁴⁸ is selected from the group consisting of halogen, CN, NO₂, OH, O(C₁-C₄-alkyl), NH₂, NH(C₁-C₄-alkyl), and N(C₁-C₄-alkyl)(C₁-C₄-alkyl), C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁵¹;
wherein R⁴⁹, R⁵⁰, R⁵¹ are independently selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein R³ is selected from the group consisting of
   (i) (L¹)_{y}-X¹,
   (ii) (L¹)_{y}-X¹-(L²)_{y}-X², and
   (iii) (L¹)_{y}-X¹-(L²)_{y}-X²-(L³)_{y}-X³; and
   (iv) (L¹)_{y}-X¹-(L²)_{y}-X²-(L³)_{y}-X³-(L⁴)_{y}-X⁴
wherein X¹, X², X³, and X⁴ are independently selected from the group consisting of H, C₁-C₆-alkyl, a 3- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein L¹, L², L³, and L⁴ are independently selected from the group consisting of C(═O), C(═O)O, C(═O)N(R^{a}), O, N(R^{a}), N(R^{a})C(═O),
wherein R^{a} is selected from the group consisting of H, C₁-C₄-alkyl, and C₁-C₄-haloalkyl, and wherein y is 0 or 1.

In a further embodiment 3(D), R³ is selected from the group consisting of H, (C₁-C₄-alkyl)OH, (C₁-C₄-alkyl)NH₂, (C₁-C₄-alkyl)NH(C₁-C₄-alkyl), (C₁-C₄-alkyl)N(C₁-C₄-alkyl)(C₁-C₄-alkyl), C(═O)NH₂, C(═O)NH(C₁-C₄-alkyl), C(═O)N(C₁-C₄-alkyl)(C₁-C₄-alkyl), C(═O)R²⁵, (C₁-C₄-alkyl)C(═O)NH₂, (C₁-C₄-alkyl)C(═O)NH(C₁-C₄-alkyl), (C₁-C₄-alkyl)C(═O)N(C₁-C₄-alkyl)(C₁-C₄-alky), and (C₁-C₄-alkyl)C(═O)R²⁷, and a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, wherein each substitutable carbon or heteroatom in the aforementioned cyclic moieties is independently unsubstituted or substituted with one or more, same or different substituents selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl), wherein R²⁵ and R²⁷ are independently a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl).

The following embodiments relate to R⁵ as defined above in the first aspect A1.

In embodiment 5(A), R⁵ is selected from the group consisting of a 5- to 6-membered partially unsaturated or fully unsaturated carbocyclic or heterocyclic ring and a 9- to 10-membered partially unsaturated or fully unsaturated carbobicyclic or heterobicyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁷. The following substituent meanings are relevant in connection with embodiment 5(A):
R¹⁷ is selected from the group consisting of
   (i) halogen, CN, NO₂, C₁-C₄-alkyl, C₂-C₄-alkenyl, C₂-C₄-alkynyl, and a 3- to 9-membered saturated, partially unsaturated or fully unsaturated carbocyclic or heterocyclic ring, wherein said heterocyclic ring comprises one or more, same or different heteroatoms selected from O, N or S, wherein said Nand/or S-atoms are independently oxidized or non-oxidized, and wherein each substitutable carbon or hetero-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R²⁰), OR²⁰, S(═O)ₙR²⁰, S(═O)ₙN(R^{20a})(R²⁰), S(═O)ₘOR²⁰, N(R^{20a})(R²⁰), N(R²⁰)C(═O)R¹⁹, N(R²⁰)C(═O)OR²⁰, N(R²⁰)C(═O)N(R^{20a})(R²⁰), N(R²⁰)S(═O)ₙ(R²⁰), N(R²⁰)S(═O)ₘN(R^{20a})(R^{20b}), and N(R²⁰)S(═O)ₘOR²⁰;
   and/or two R¹⁷ present on one C-atom together form ═O;
R¹⁸ is selected from the group consisting of
   (i) halogen, CN, NO₂, C₁-C₄-alkyl, C₁-C₄-haloalkyl, C₂-C₄-alkenyl, C₂-C₄-haloalkenyl, C₂-C₄-alkynyl, and C₂—C-haloalkynyl;
   (ii) C(═O)R²³, C(═O)OR²⁴, C(═O)SR²⁴, C(═O)N(R^{24a})(R^{24b}), OR²⁴, S(═O)ₙR²⁴, S(═O)ₙN(R^{24a})(R^{24b}), S(═O)ₘOR²⁴, N(R^{24a})(R^{24b}), N(R²⁴)C(═O)R²³, N(R²⁴)C(═O)OR²⁴, N(R²⁴)C(═O)N(R^{24a})(R^{24b}), N(R²⁴)S(═O)ₙ(R²⁴), N(R²⁴)S(═O)ₘN(R^{24a})(R^{24b}), and N(R²⁴)S(═O)ₘOR²⁴;
R²³, R²⁴, R^{24a}, R^{24b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
and wherein
n is 0, 1 or 2; and
m is 1 or 2.

In a preferred embodiment 5(B), R⁵ is selected from the group consisting of a 5- to 6-membered partially unsaturated or fully unsaturated carbocyclic or heterocyclic ring and a 9- to 10-membered partially unsaturated or fully unsaturated carbobicyclic or heterobicyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁷; wherein R¹⁷ is selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, C(═O)R¹⁹, C(═O)OR²⁰, C(═O)N(R^{20a})(R^{20b}), OR²⁰, N(R^{20a})(R^{20b}), N(R²⁰)C(═O)R¹⁹, N(R²⁰)C(═O)OR²⁰, and N(R²⁰)C(═O)N(R^{20a})(R^{20b}); and/or two R¹⁷ present on one C-atom together form ═O;

wherein R¹⁹, R²⁰, R^{20a}, R^{20b} are independently selected from the group consisting of H, C₁-C₂-alkyl, and C₁-C₂-haloalkyl.

In another embodiment 5(C), R⁵ has the formula (S1)

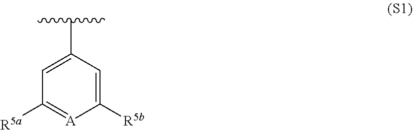

and wherein
   A is N or CR⁵;
wherein R^{5a}, R^{5b}, R^{5c} are independently selected from the group consisting of
   (i) H, halogen, CN, NO₂, C₁-C₄-alkyl, C₂-C₄-alkenyl, and C₂-C₄-alkynyl, wherein each substitutable carbon-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
      with the proviso that at least one of R^{5a}, R^{5b}, R^{5c} is not H;
or
R^{5a} is selected from the group consisting of
   (i) H, halogen, CN, NO₂, C₁-C₄-alkyl, C₂-C₄-alkenyl, and C₂-C₄-alkynyl, wherein each substitutable carbon-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
      and
R^{5b} and R^{5c} together with the atoms to which they are attached form a 5- to 6-membered partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is independently unsubstituted or substituted with one or more, same or different substituents selected from the group consisting of
   (i) H, halogen, CN, NO₂, C₁-C₄-alkyl, C₂-C₄-alkenyl, and C₂-C₄-alkynyl, wherein each substitutable carbon-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
and wherein
n is 0, 1 or 2; and
m is 1 or 2.

In another embodiment 5(D), R⁵ is selected from the group consisting of a 6-membered fully unsaturated carbocyclic or heterocyclic ring and a 9- to 10-membered fully unsaturated heterobicyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is independently substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, C(═O)R¹⁹, C(═O)OR²⁰, C(═O)N(R^{20a})(R^{20b}), OR²⁰, N(R^{20a})(R^{20b}), N(R²⁰)C(═O)R¹⁹, N(R²⁰)C(═O)OR²⁰, and N(R²⁰)C(═O)N(R^{20a})(R^{20b}); and wherein each substitutable carbon or heteroatom in the aforementioned bicyclic moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, C(═O)R¹⁹, C(═O)OR²⁰, C(═O)N(R^{20a})(R^{20b}), OR²⁰, N(R^{20a})(R^{20b}), N(R²⁰)C(═O)R¹⁹, N(R²⁰)C(═O)OR²⁰, and N(R²⁰)C(═O)N(R^{20a})(R^{20b});

wherein R¹⁹, R²⁰, R^{20a}, R^{20b} are independently selected from the group consisting of H, C₁-C₂-alkyl, and C₁-C₂-haloalkyl.

In another embodiment 5(E), R⁵ is selected from the group consisting of a 5- to 6-membered partially unsaturated or fully unsaturated carbocyclic or heterocyclic ring and a 9- to 10-membered partially unsaturated or fully unsaturated carbobicyclic or heterobicyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁷; wherein R¹⁷ is selected from the group consisting of halogen, CN, NO₂, C₁-C₄-alkyl, C₁-C₄-haloalkyl, OH, O(C₁-C₄-alkyl), NH₂, NH(C₁-C₄-alkyl), and N(C₁-C₄-alkyl)(C₁-C₄-alkyl); and/or two R¹⁷ present on one C-atom together form ═O.

In another embodiment 5(F), R⁵ is selected from the group consisting of a 5- to 6-membered partially unsaturated or fully unsaturated carbocyclic or heterocyclic ring and a 9- to 10-membered partially unsaturated or fully unsaturated carbobicyclic or heterobicyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁷; wherein R¹⁷ is selected from the group consisting of halogen, CN, NO₂, C₁-C₂-alkyl, C₁-C₂-haloalkyl, OH, O(C₁-C₂-alkyl), NH₂, NH(C₁-C₂-alkyl), and N(C₁-C₂-alkyl)(C₁-C₂-alkyl); and/or two R¹⁷ present on one C-atom together form ═O.

### Detailed Description Relating to Aspects B1 to B5

In the following, preferred embodiments of the substituents in the above formula (I) are described in further detail.

The following embodiments relate to R¹ as defined above in the first aspect B1.

In embodiment 1(A), R¹ is a 3- to 9-membered saturated, partially unsaturated or fully unsaturated carbocyclic or heterocyclic ring or a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁶; wherein R⁶ is selected from the group consisting of
(i) halogen, CN, NO₂, C₁-C₆-alkyl, C₂-C₆-alkenyl, and C₂-C₆-alkynyl, wherein each substitutable carbon or hetero-atom in the aforementioned moieties is unsubstituted or substituted with one or more, same or different substituents R⁷;
(ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²; and
wherein R⁷ is selected from the group consisting of
(i) halogen, CN, NO₂, C₁-C₆-alkyl, C₂-C₆-alkenyl, and C₂-C₆-alkynyl,
(ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹².

The following substituent meanings are relevant in connection with embodiment 1(A):
R¹¹, R¹², R^{12a}, R^{12b} are independently selected from the group consisting of
   (i) H, C₁-C₆-alkyl, C₂-C₆-alkenyl, and C₂-C₆-alkynyl, wherein each substitutable carbon atom in the aforementioned moieties is independently unsubstituted or substituted with one or more, same or different substituents R¹³;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁴;
R¹³ is selected from the group consisting of
   (i) halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, N(R^{15a})(R^{15b}), C(═O)NR^{15a}R^{15b}, S(═O)ₙNR^{15a}R^{15b}, OR¹⁵ and S(═O)ₙR¹⁵;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁴;
R¹⁴ is selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C(═O)R¹⁶, C(═O)OR¹⁵, C(═O)SR¹⁵, C(═O)N(R^{15a})(R^{15b}), OR¹⁵, S(═O)ₙR¹⁵, S(═O)ₙN(R^{15a})(R^{15b}), S(═O)ₘOR¹⁵, N(R^{15a})(R^{15b}), N(R¹⁵)C(═O)R¹⁶, N(R¹⁵)C(═O)OR¹⁵, N(R¹⁵)C(═O)N(R^{15a})(R^{15b}), N(R¹⁵)S(═O)ₙ(R¹⁵), N(R¹⁵)S(═O)ₘN(R^{15a})(R^{15b}), and N(R¹⁵)S(═O)ₘOR¹⁵;
R¹⁵, R^{15a}, R^{15b}, R¹⁶ are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
and wherein
n is 0, 1 or 2; and
m is 1 or 2.

Preferably, the following substituent meanings are relevant in connection with embodiment 1(A):
R¹¹, R¹², R^{12a}, R^{12b} are independently selected from the group consisting of H, C₁-C₃-alkyl, C₂-C₃-alkenyl and C₂-C₃-alkynyl.

In a preferred embodiment 1(B), R¹ is a 5- to 6-membered fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl.

In a further embodiment 1(C), R¹ is a 5- to 6-membered fully unsaturated carbocyclic or heterocyclic ring, wherein said heterocyclic or ring comprises one or more, same or different heteroatoms selected from O, N or S, wherein said N- and/or S-atoms are independently oxidized or non-oxidized, and wherein each substitutable carbon or heteroatom in the aforementioned cyclic moieties is independently unsubstituted or substituted with one or more, same or different substituents selected from the group consisting of halogen, CN, and trifluoromethyl.

In a further embodiment 1(D), R¹ is phenyl.

In a further embodiment 1(E), R¹ is 4-fluorophenyl.

In a further embodiment 1(F), R¹ is 3-fluorophenyl.

In a further embodiment 1(G), R¹ is 4-cyanophenyl.

In a further embodiment 1(H), R¹ is 3-cyanophenyl.

In a further embodiment 1(1), R¹ is 4-(trifluoromethyl)phenyl.

In a further embodiment 1(J), R¹ is furan-2-yl.

The following embodiments relate to R² as defined above in the first aspect B1.

In embodiment 2(A), R² is halogen, NH₂, NH(C₁-C₃-alkyl), or N(C₁-C₃-alkyl)(C₁-C₃-alkyl).
In a preferred embodiment 2(B), R² is halogen, NH₂, NH(CH₃), or N(CH₃)₂.
In a more preferred embodiment 2(C), R² is NH₂.

The following embodiments relate to R³ as defined above in the first aspect B1.
In embodiment 3(A), R³ is
   (i) H, halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring or a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁸;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
wherein R⁸ is selected from the group consisting of
   (i) halogen, CN, NO₂, C₁-C₆-alkyl, C₂-C₆-alkenyl, and C₂-C₆-alkynyl, wherein each substitutable carbon or hetero-atom in the aforementioned moieties is unsubstituted or substituted with one or more, same or different substituents R⁹;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²; and
wherein R⁹ is selected from the group consisting of
   (i) halogen, CN, NO₂, C₁-C₆-alkyl, C₂-C₆-alkenyl, and C₂-C₆-alkynyl,
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹².
In a preferred embodiment 3(B), R³ is
   (i) H, halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring or a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;

The following substituent meanings are relevant in connection with embodiments 3(A) and 3(B):
R¹¹, R¹², R^{12a}, R^{12b} are independently selected from the group consisting of
   (i) H, C₁-C₆-alkyl, C₂-C₆-alkenyl, and C₂-C₆-alkynyl, wherein each substitutable carbon atom in the aforementioned moieties is independently unsubstituted or substituted with one or more, same or different substituents R¹³;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁴;
R¹³ is selected from the group consisting of
   (i) halogen, CN, NO₂, C₁-C₆-alkyl, C₁—C-haloalkyl, C₂-C₆-alkenyl, C₂-C₆-haloalkenyl, C₂-C₆-alkynyl, C₂—C-haloalkynyl, N(R^{15a})(R^{15b}), C(═O)NR^{15a}R^{15b}, S(═O)ₙNR^{15a}R^{15b}, OR¹⁵ and S(═O)ₙR¹⁵;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁴;
R¹⁴ is selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C(═O)R¹⁶, C(═O)OR¹⁵, C(═O)SR¹⁵, C(═O)N(R^{15a})(R^{15b}), OR¹⁵, S(═O)ₙR¹⁵, S(═O)ₙN(R^{15a})(R^{15b}), S(═O)ₘOR¹⁵, N(R^{15a})(R^{15b}), N(R¹⁵)C(═O)R¹⁶, N(R¹⁵)C(═O)OR¹⁵, N(R¹⁵)C(═O)N(R^{15a})(R^{15b}), N(R¹⁵)S(═O)ₙ(R¹⁵), N(R¹⁵)S(═O)ₘN(R^{15a})(R^{15b}), and N(R¹⁵)S(═O)ₘOR¹⁵;
R¹⁵, R^{15a}, R^{15b}, R¹⁶ are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
and wherein
n is 0, 1 or 2; and
m is 1 or 2.

Preferably, the following substituent meanings are relevant in connection with embodiments 3(A) and 3(B):
R¹¹, R¹², R^{12a}, R^{12b} are independently selected from the group consisting of H, C₁-C₃-alkyl, C₂-C₃-alkenyl and C₂-C₃-alkynyl.

In a further embodiment 3(C), R³ is
(i) H or a 6-membered saturated, partially unsaturated or fully unsaturated carbocyclic ring, wherein each substitutable carbonatom in the aforementioned moieties is independently unsubstituted or substituted with one or more, same or different substituents selected from the group consisting of CN and NO₂; or
(ii) C(═O)NH₂.

In a further embodiment 3(D), R³ is H.

In a further embodiment 3(E), R³ is 3-nitrophenyl.

In a further embodiment 3(F), R³ is 3-cyanophenyl.

In a further embodiment 3(G), R³ is cyclohexyl.

In a further embodiment 3(H), R³ is C(═O)NH₂.

The following embodiments relate to R⁵ as defined above in the first aspect B1.

In embodiment 5(A), R⁵ is a 5- to 6-membered saturated, partially unsaturated or fully unsaturated carbocyclic or heterocyclic ring or a 9- to 10-membered saturated, partially unsaturated or fully unsaturated carbobicyclic or heterobicyclic ring, wherein said heterocyclic or heterobicyclic ring comprises one or more N-atoms, and wherein said N-atoms are independently oxidized or non-oxidized, and wherein each substitutable carbon or heteroatom in the aforementioned cyclic moieties is independently substituted with one or more, same or different substituents R¹⁷, and wherein each substitutable carbon or heteroatom in the aforementioned bicyclic moieties is independently unsubstituted or substituted with one or more, same or different substituents R¹⁷.

In a preferred embodiment 5(B), R⁵ is a 5- to 6-membered fully unsaturated carbocyclic or heterocyclic ring or a 9- to 10-membered fully unsaturated carbobicyclic or heterobicyclic ring, wherein said heterocyclic or heterobicyclic ring comprises one or more N-atoms, and wherein said N-atoms are independently oxidized or non-oxidized, and wherein each substitutable carbon or heteroatom in the aforementioned cyclic moieties is independently substituted with one or more, same or different substituents R¹⁷, and wherein each substitutable carbon or heteroatom in the aforementioned bicyclic moieties is independently unsubstituted or substituted with one or more, same or different substituents R¹⁷.

The following substituent meanings are relevant in connection with embodiments 5(A) and 5(B):
R¹⁷ is selected from the group consisting of
   (i) halogen, CN, NO₂, C₁-C₄-alkyl, C₂-C₄-alkenyl, and C₂-C₄-alkynyl, wherein each substitutable carbon or hetero-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R²⁰), OR²⁰, S(═O)ₙR²⁰, S(═O)ₙN(R^{20a})(R²⁰), S(═O)ₘOR²⁰, N(R^{20a})(R²⁰), N(R²⁰)C(═O)R¹⁹, N(R²⁰)C(═O)OR²⁰, N(R²⁰)C(═O)N(R^{20a})(R²⁰), N(R²⁰)S(═O)ₙ(R²⁰), N(R²⁰)S(═O)ₘN(R^{20a})(R^{20b}), and N(R²⁰)S(═O)ₘOR²⁰;
R¹⁸ is selected from the group consisting of halogen, N(R^{20a})(R²⁰), and OR²⁰;
R¹⁹, R²⁰, R^{20a}, R^{20b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
and wherein
n is 0, 1 or 2; and
m is 1 or 2.

Preferably, the following substituent meanings are relevant in connection with embodiments 5(A) and 5(B):
R¹⁷ is selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, OR²⁰, and N(R^{20a})(R^{20b});
R²⁰R^{20a}, R^{20b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;

In a more preferred embodiment 5(C), R⁵ has the formula (S1)

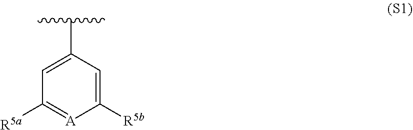

and wherein
   A is N or CR^{5c};
R^{5a}, R^{5b}, R^{5c} are independently selected from the group consisting of
   (i) H, halogen, CN, NO₂, C₁-C₄-alkyl, C₂-C₄-alkenyl, and C₂-C₄-alkynyl, wherein each substitutable carbon or hetero-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
      with the proviso that at least one of R^{5a}, R^{5b}, R^{5c} is not H;
or
R^{5a} is selected from the group consisting of
   (i) H, halogen, CN, NO₂, C₁-C₄-alkyl, C₂-C₄-alkenyl, and C₂-C₄-alkynyl, wherein each substitutable carbon or hetero-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
      and
R^{5b} and R^{5c} together with the atoms to which they are attached form a 5- to 6-membered fully unsaturated carbocyclic or heterocyclic ring, wherein said heterocyclic ring comprises one or more N-atoms, and wherein said N-atoms are independently oxidized or non-oxidized, and wherein each substitutable carbon or heteroatom in the aforementioned cyclic moieties is independently unsubstituted or substituted with one or more, same or different substituents selected from the group consisting of
   (i) H, halogen, CN, NO₂, C₁-C₄-alkyl, C₂-C₄-alkenyl, and C₂-C₄-alkynyl, wherein each substitutable carbon or hetero-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;

In a more preferred embodiment 5(D), R⁵ has the formula (S1)

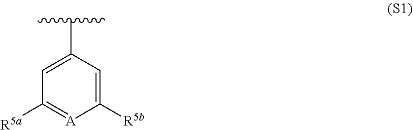

and wherein
   A is N or CR⁵;
R^{5a}, R^{5b}, R^{5c} are independently selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, OR²⁰, and N(R^{20a})(R^{20b})
or
R^{5a} is selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, OR²⁰, and N(R^{20a})(R^{20b})
and
R^{5b} and R^{5c} together with the atoms to which they are attached form a 5- to 6-membered fully unsaturated carbocyclic or heterocyclic ring, wherein said heterocyclic ring comprises one or more N-atoms, and wherein said N-atoms are independently oxidized or non-oxidized, and wherein each substitutable carbon or heteroatom in the aforementioned cyclic moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, OR²⁰, and N(R^{20a})(R^{20b});

In a more preferred embodiment 5(E), R⁵ is a 6-membered fully unsaturated carbocyclic or heterocyclic ring or a 9- to 10-membered fully unsaturated heterobicyclic ring, wherein said heterocyclic or heterobicyclic ring comprises one or more N-atoms, and wherein said N-atoms are independently oxidized or non-oxidized, and wherein each substitutable carbon or heteroatom in the aforementioned cyclic moieties is independently substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, OR²⁰, and N(R^{20a})(R^{20b}), and wherein each substitutable carbon or heteroatom in the aforementioned bicyclic moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, OR²⁰, and N(R^{20a})(R^{20b});

The following substituent meanings are relevant in connection with embodiments 5(C), 5(D) and 5(E):
R¹⁸ is selected from the group consisting of halogen, N(R^{20a})(R^{20b}), and OR²⁰;
R²⁰, R^{20a}, R^{20b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;

In a further embodiment 5(F), R⁵ is a 6-membered fully unsaturated carbocyclic or heterocyclic ring or a 9- to 10-membered fully unsaturated heterobicyclic ring, wherein said heterocyclic or heterobicyclic ring comprises one or more N-atoms, and wherein said N-atoms are independently oxidized or non-oxidized, and wherein each substitutable carbon or heteroatom in the aforementioned cyclic moieties is independently substituted with one or more, same or different substituents selected from the group consisting of halogen, —OH, —OCH₃, —CH₃, —CF₃, and —NHCH₃, and wherein each substitutable carbon or heteroatom in the aforementioned bicyclic moieties is independently unsubstituted or substituted with one or more, same or different substituents selected from the group consisting of halogen, —OH, —OCH₃, —CH₃, —CF₃, and —NHCH₃.

In a further embodiment 5(G), R⁵ is 4-hydroxyphenyl.
In a further embodiment 5(H), R⁵ is 3-chloro-4-hydroxyphenyl.
In a further embodiment 5(I), R⁵ is 3,5-dichloro-4-hydroxyphenyl.
In a further embodiment 5(J), R⁵ is 3-bromo-4-hydroxy-5-chlorophenyl.
In a further embodiment 5(K), R⁵ is 2,6-dimethylpyridin-4-yl.
In a further embodiment 5(L), R⁵ is 2-(N-methylamino)pyridin-4-yl.
In a further embodiment 5(M), R⁵ is 2-methyl-6-(trifluoromethyl)pyridin-4-yl.
In a further embodiment 5(N), R⁵ is quinolin-6-yl.
In a further embodiment 5(O), R⁵ is 1H-indazol-5-yl.

It is to be understood that the above embodiments 1(D)-1(J) regarding R¹, 2(C) regarding R², 3(D)-3(H) regarding R³ and 5(G)-5(O) regarding R⁵, are also disclosed in combination with each other.

The following combinations of embodiments of the first aspect B1, which are summarized in Table 1 are preferred.

TABLE 1

| Line | R¹ | R² | R³ | R⁵ |
|---|---|---|---|---|
| 1 | 1(B) | 2(B) | 3(C) | 5(C) |
| 1 | 1(C) | 2(B) | 3(C) | 5(C) |
| 1 | 1(D) | 2(B) | 3(C) | 5(C) |
| 1 | 1(B) | 2(C) | 3(C) | 5(C) |
| 1 | 1(C) | 2(C) | 3(C) | 5(C) |
| 1 | 1(D) | 2(C) | 3(C) | 5(C) |
| 1 | 1(B) | 2(B) | 3(D) | 5(C) |
| 1 | 1(C) | 2(B) | 3(D) | 5(C) |
| 1 | 1(D) | 2(B) | 3(D) | 5(C) |
| 1 | 1(B) | 2(C) | 3(D) | 5(C) |
| 1 | 1(C) | 2(C) | 3(D) | 5(C) |
| 1 | 1(D) | 2(C) | 3(D) | 5(C) |
| 1 | 1(B) | 2(B) | 3(C) | 5(H) |
| 1 | 1(C) | 2(B) | 3(C) | 5(H) |
| 1 | 1(D) | 2(B) | 3(C) | 5(H) |
| 1 | 1(B) | 2(C) | 3(C) | 5(H) |
| 1 | 1(C) | 2(C) | 3(C) | 5(H) |
| 1 | 1(D) | 2(C) | 3(C) | 5(H) |
| 1 | 1(B) | 2(B) | 3(D) | 5(H) |
| 1 | 1(C) | 2(B) | 3(D) | 5(H) |
| 1 | 1(D) | 2(B) | 3(D) | 5(H) |
| 1 | 1(B) | 2(C) | 3(D) | 5(H) |
| 1 | 1(C) | 2(C) | 3(D) | 5(H) |
| 1 | 1(D) | 2(C) | 3(D) | 5(H) |

### Definitions Relating to Aspects A1 to A5 and B1 to B5

The term “compound(s) of the present invention” is to be understood as equivalent to the term “compound(s) according to the invention”, therefore also comprising a salt, stereoisomer, tautomer, isotopologue, or N-oxide thereof.

The compounds according to the invention may be amorphous or may exist in one or more different crystalline states (polymorphs) which may have different macroscopic properties such as stability or show different biological properties such as activities. The present invention relates to amorphous and crystalline compounds of formula (I), mixtures of different crystalline states of the respective compound of the invention, as well as amorphous or crystalline salts thereof.

Salts of the compounds according to the invention are preferably pharmaceutically acceptable salts, such as those containing counterions present in drug products listed in the US FDA Orange Book database. They can be formed in a customary manner, e.g., by reacting the compound with an acid of the anion in question if the compounds according to the invention have a basic functionality or by reacting acidic compounds according to the invention with a suitable base.

Suitable cationic counterions are in particular the ions of the alkali metals, preferably lithium, sodium and potassium, of the alkaline earth metals, preferably calcium, magnesium and barium, and of the transition metals, preferably manganese, copper, silver, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethyl-ammonium, bis(2-hydroxyethyl)ammonium, benzyltrimethylammonium and benzyltriethylammonium, furthermore the cations of 1,4-piperazine, meglumine, benzathine and lysine.

Suitable acidic counterions are in particular chloride, bromide, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate, furthermore lactate, gluconate, and poly acids such as succinate, oxalate, maleate, fumarate, malate, tartrate and citrate, furthermore sulfonate anions such as besylate (benzenesulfonate), tosylate (p-toluenesulfonate), napsylate (naphthalene-2-sulfonate), mesylate (methanesulfonate), esylate (ethanesulfonate), and ethanedisulfonate. They can be formed by reacting compounds according to the invention that have a basic functionality with an acid of the corresponding anion.

Depending on the substitution pattern, the compounds according to the invention may have one or more centres of chirality, including axial chirality. The invention provides both pure enantiomers or pure diastereomers of the compounds according to the invention, and their mixtures, including racemic mixtures. Suitable compounds according to the invention also include all possible geometrical stereoisomers (cis/trans isomers or E/Z isomers) and mixtures thereof. Cis/trans isomers may be present with respect to, e.g., an alkene, carbon-nitrogen double-bond or amide group.

Tautomers may be formed, if a substituent is present at the compound of formula (I), which allows for the formation of tautomers such as keto-enol tautomers, imine-enamine tautomers, amide-imidic acid tautomers or the like.

An isotopologue is an isotopically enriched compound. The term “isotopically enriched compound” refers to a compound containing at least one atom having an isotopic composition other than the natural isotopic composition of that atom. Preferably, the isotopologue is a deuteriumenriched compound.

The term “N-oxide” includes any compound of the present invention which has at least one tertiary nitrogen atom that is oxidized to a N-oxide moiety.

The term “substituted”, as used herein, means that a hydrogen atom bonded to a designated atom is replaced with a specified substituent, provided that the substitution results in a stable or chemically feasible compound. Unless otherwise indicated, a substituted atom may have one or more substituents and each substituent is independently selected.

The term “substitutable”, when used in reference to a designated atom, means that attached to the atom is a hydrogen, which can be replaced with a suitable substituent.

When it is referred to certain atoms or moieties being substituted with “one or more” substituents, the term “one or more” is intended to cover at least one substituent, e.g. 1 to 10 substituents, preferably 1, 2, 3, 4, or 5 substituents, more preferably 1, 2, or 3 substituents, most preferably 1, or 2 substituents. When neither the term “unsubstituted” nor “substituted” is explicitly mentioned concerning a moiety, said moiety is to be considered as unsubstituted.

The organic moieties mentioned in the above definitions of the variables are—like the term halogen—collective terms for individual listings of the individual group members. The prefix Cₙ-Cₘ indicates in each case the possible number of carbon atoms in the group.

The term “halogen” denotes in each case fluorine, bromine, chlorine or iodine, in particular fluorine or chlorine.

The term “alkyl” as used herein denotes in each case a straight-chain or branched alkyl group having usually from 1 to 6 carbon atoms, preferably 1 to 5 or 1 to 4 carbon atoms, more preferably 1 to 3 or 1 to 2 or 1 carbon atoms. Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl.

The term “haloalkyl” as used herein denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with halogen atoms. Preferred haloalkyl moieties are selected from C₁-C₄-haloalkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “alkenyl” as used herein denotes in each case an unsaturated hydrocarbon group having usually 2 to 6, preferably 2 to 4 carbon atoms comprising at least one carbon-carbon double bond in any position, e.g. vinyl (ethenyl), allyl (2-propen-1-yl), 1-propen-1-yl, 2-propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like. If geometric isomers are possible with regard to the double bond, the present invention relates to both, the E- and Z-isomers. Preferred alkenyl groups according to the invention are terminal alkenyl groups. The bonding of vinyl is exemplified below.

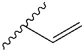 [SMILES: *C=C]

The term “haloalkenyl” as used herein refers to an alkenyl group as defined above, wherein the hydrogen atoms are partially or totally replaced with halogen atoms.

The term “alkynyl” as used herein denotes in each case an unsaturated hydrocarbon group having usually 2 to 6, preferably 2 to 5 or 2 to 4 carbon atoms, more preferably 2 to 3 carbon atoms, comprising at least one carbon-carbon triple bond in any position, e.g. ethynyl, propargyl (2-propyn-1-yl), 1-propyn-1-yl, 1-methylprop-2-yn-1-yl), 2-butyn-1-yl, 3-butyn-1-yl, 1-pentyn-1-yl, 3-pentyn-1-yl, 4-pentyn-1-yl, 1-methylbut-2-yn-1-yl, 1-ethylprop-2-yn-1-yl and the like.

The term “haloalkynyl” as used herein refers to an alkynyl group as defined above, wherein the hydrogen atoms are partially or totally replaced with halogen atoms.

The term “alkoxy” as used herein denotes in each case a straight-chain or branched alkyl group which is bonded via an oxygen atom and has usually from 1 to 6 carbon atoms, preferably 1 to 2 carbon atoms, more preferably 1 carbon atom. Examples of an alkoxy group are methoxy, ethoxy, n-propoxy, iso-propoxy, n-butyloxy, 2-butyloxy, iso-butyloxy, tert.-butyloxy, and the like.

The term “haloalkoxy” as used herein denotes in each case a straight-chain or branched alkoxy group having from 1 to 6 carbon atoms, preferably 1 to 2 carbon atoms, more preferably 1 carbon atom, wherein the hydrogen atoms of this group are partially or totally replaced with halogen atoms, in particular fluorine atoms. Preferred haloalkoxy moieties include C₁-haloalkoxy, in particular C₁-fluoroalkoxy, such as trifluoromethoxy and the like.

The term “carbocyclic” includes, unless otherwise indicated, in general a 3- to 9-membered, preferably a 4- to 8-membered or a 5- to 7-membered, more preferably a 5- or 6-membered monocyclic ring comprising 3 to 9, preferably 4 to 8 or 5 to 7, more preferably 5 or 6 carbon atoms. The carbocycle may be saturated, partially unsaturated, or fully unsaturated. Preferably, the term “carbocycle” covers cycloalkyl and cycloalkenyl groups as defined above, for example cyclopropane, cyclobutane, cyclopentane and cyclohexane rings. When it is referred to “fully unsaturated” carbocycles, this term also includes “aromatic” carbocycles or aryls. In certain preferred embodiments, a fully unsaturated carbocycle is an aromatic carbocycle as defined below, preferably a 6-membered aromatic carbocycle. Phenyl is a preferred fully unsaturated carbocycle.

The term “carbobicyclic” includes in general bicyclic 6 to 14-membered, preferably 7- to 12-membered or 8- to 10-membered, more preferably 9- or 10-membered bicyclic rings comprising 6 to 14, preferably 7 to 12 or 8 to 10, more preferably 9 or 10 carbon atoms. The carbobicycle may be saturated, partially unsaturated, or fully unsaturated. Preferably, the term “carbobicycle” covers bicycloalkyl, bicycloalkenyl and bicyclic aromatic groups, for example bicyclohexane (decalin), bicycloheptane (such as norbornane), bicyclooctane (such as bicyclo[2.2.2]octane, bicyclo[3.2.1]octane or bicyclo[4.2.0]octane), bicyclononane (such as bicyclo[3.3.1]nonane or bicyclo[4.3.0]nonane), bicyclodecane (such as bicyclo[4.4.0]decane), bicycloundecane (such as bicyclo[3.3.3]undecane), norbornene, naphthalene and the like. Preferably, the carbobicycle is a fused carbobicycle, for example naphthalene and the like.

The term “heterocyclic” includes, unless otherwise indicated, in general a 3- to 9-membered, preferably a 4- to 8-membered or 5- to 7-membered, more preferably 5- or 6-membered, in particular 6-membered monocyclic ring. The heterocycle may be saturated, partially unsaturated, or fully unsaturated. As used in this context, the term “fully unsaturated” also includes “aromatic”. In a preferred embodiment, a fully unsaturated heterocycle is thus an aromatic heterocycle, preferably a 5- or 6-membered aromatic heterocycle comprising one or more, e.g. 1, 2, 3, or 4, preferably 1, 2, or 3 heteroatoms selected from N, O and S as ring members, where S-atoms as ring members may be present as S, SO or SO₂. Examples of aromatic heterocycles are provided below in connection with the definition of “hetaryl”. “Hetaryls” or “heteroaryls” are covered by the term “heterocycles”. The saturated or partially unsaturated heterocycles usually comprise 1, 2, 3, 4 or 5, preferably 1, 2 or 3 heteroatoms selected from N, O and S as ring members, where S-atoms as ring members may be present as S, SO or SO₂. Preferably, the S atom will not be present in oxidized form in fully unsaturated compounds. In particular, the following scenarios are covered:

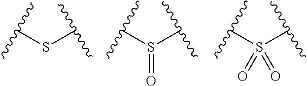

A skilled person is aware that resonance structures of the oxidized forms may be possible.

Saturated heterocycles include, unless otherwise indicated, in general 3- to 9-membered, preferably 4- to 8-membered or 5- to 7-membered, more preferably 5- or 6-membered monocyclic rings comprising 3 to 9, preferably 4 to 8 or 5 to 7, more preferably 5 or 6 atoms comprising at least one heteroatom, such as pyrrolidine, tetrahydrothiophene, tetrahydrofuran, piperidine, tetrahydropyran, dioxane, morpholine or piperazine.

The term “hetaryl” or “heteroaryl” or “aromatic heterocycle” or “aromatic heterocyclic ring” includes monocyclic 5- or 6-membered aromatic heterocycles comprising as ring members 1, 2, 3 or 4 heteroatoms selected from N, O and S, where S-atoms as ring members may be present as S, SO or SO₂. Preferably, the S atom will not be present in oxidized form in fully unsaturated compounds. In particular, the following scenarios are covered:

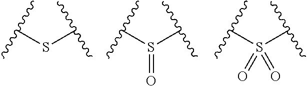

A skilled person is aware that resonance structures of the oxidized forms may be possible. Examples of 5- or 6-membered aromatic heterocycles include pyridyl, i.e. 2-, 3-, or 4-pyridyl, pyrimidinyl, i.e. 2-, 4- or 5-pyrimidinyl, pyrazinyl, pyridazinyl, i.e. 3- or 4-pyridazinyl, thienyl, i.e. 2- or 3-thienyl, furyl, i.e. 2- or 3-furyl, pyrrolyl, i.e. 2- or 3-pyrrolyl, oxazolyl, i.e. 2-, 3- or 5-oxazolyl, isoxazolyl, i.e. 3-, 4- or 5-isoxazolyl, thiazolyl, i.e. 2-, 3- or 5-thiazolyl, isothiazolyl, i.e. 3-, 4- or 5-isothiazolyl, pyrazolyl, i.e. 1-, 3-, 4- or 5-pyrazolyl, i.e. 1-, 2-, 4- or 5-imidazolyl, oxadiazolyl, e.g. 2- or 5-[1,3,4]oxadiazolyl, 4- or 5-(1,2,3-oxadiazol)yl, 3- or 5-(1,2,4-oxadiazol)yl, 2- or 5-(1,3,4-thiadiazol)yl, thiadiazolyl, e.g. 2- or 5-(1,3,4-thiadiazol)yl, 4- or 5-(1,2,3-thiadiazol)yl, 3- or 5-(1,2,4-thiadiazol)yl, triazolyl, e.g. 1H-, 2H- or 3H-1,2,3-triazol-4-yl, 2H-triazol-3-yl, 1H-, 2H-, or 4H-1,2,4-triazolyl and tetrazolyl, i.e. 1H- or 2H-tetrazolyl.

The term “heterobicyclic” includes in general bicyclic 6 to 14-membered, preferably 7- to 12-membered or 8- to 10-membered, more preferably 9- or 10-membered bicyclic rings comprising as ring members 1, 2, 3 or 4 heteroatoms selected from N, O and S, where S-atoms as ring members may be present as S, SO or SO₂. The heterobicycle may be saturated, partially unsaturated, or fully unsaturated. Examples of heterobicycles include benzofuranyl, benzothienyl, indolyl, indazolyl, benzimidazolyl, benzoxathiazolyl, benzoxadiazolyl, benzothiadiazolyl, benzoxazinyl, quinolinyl, isoquinolinyl, purinyl, 1,8-naphthyridyl, pteridyl, pyrido[3,2-d]pyrimidyl, pyridoimidazolyl, triethylenediamine or quinuclidine and the like. Preferably, the heterobicycle is a fused heterobicycle, for example quinolinyl and the like.

As used in the specification and the claims, the singular forms of “a” and “an” also include the corresponding plurals unless the context clearly dictates otherwise. The same applies for plural forms used herein, which also include the singular forms unless the context clearly dictates otherwise.

The terms “about” and “approximately” in the context of the present invention denotes an interval of accuracy that a person skilled in the art will understand to still ensure the technical effect of the feature in question. The term typically indicates a deviation from the indicated numerical value of ±10% and preferably ±5%.

It needs to be understood that the term “comprising” is not limiting. For the purposes of the present invention, the term “consisting of” is considered to be a preferred embodiment of the term “comprising of”. If hereinafter a group is defined to comprise at least a certain number of embodiments, this is also meant to encompass a group which preferably consists of these embodiments only.

The term “pharmaceutically acceptable excipient” as used herein refers to compounds commonly comprised in pharmaceutical compositions, which are known to the skilled person. Examples of suitable excipients are exemplary listed below. Typically, a pharmaceutically acceptable excipient can be defined as being pharmaceutically inactive.

The term “treatment” is to be understood as also including the option of “prophylaxis”. Thus, whenever reference is made herein to a “treatment” or “treating”, this is to be understood as “treatment and/or prophylaxis” or “treating and/or preventing”.

### Description of Pharmaceutical Compositions According to the Present Invention

A pharmaceutical composition according to the present invention may be formulated for oral, buccal, nasal, rectal, topical, transdermal or parenteral application. Oral application may be preferred. Parenteral application can also be preferred and includes intravenous, intraarterial, intratumoral, intrathecal, intravesical, intramuscular or subcutaneous administration. The compound according to formula (I) should be applied in pharmaceutically effective amounts, for example in the amounts as set out herein below.

A pharmaceutical composition of the present invention may also be designated as formulation or dosage form. A compound of formula (I) may also be designated in the following as (pharmaceutically) active agent or active compound.

Pharmaceutical compositions may be solid or liquid dosage forms or may have an intermediate, e.g. gel-like character depending inter alia on the route of administration.

In general, the inventive dosage forms can comprise various pharmaceutically acceptable excipients, which will be selected depending on which functionality is to be achieved for the dosage form. A “pharmaceutically acceptable excipient” in the meaning of the present invention can be any substance used for the preparation of pharmaceutical dosage forms, including coating materials, film-forming materials, fillers, disintegrating agents, release-modifying materials, carrier materials, diluents, binding agents and other adjuvants. Typical pharmaceutically acceptable excipients include substances like sucrose, mannitol, sorbitol, starch and starch derivatives, lactose, and lubricating agents such as magnesium stearate, disintegrants and buffering agents.

The term “carrier” denotes pharmaceutically acceptable organic or inorganic carrier substances with which the active ingredient is combined to facilitate the application. Suitable pharmaceutically acceptable carriers include, for instance, water, aqueous salt solutions, alcohols, oils, preferably vegetable oils, propylene glycol, polyoxyethelene sorbitans, polyethylene-polypropylene block co-polymers such as poloxamer 188 or poloxamer 407, polyethylene glycols such as polyethylene glycol 200, 300, 400, 600, etc., gelatin, lactose, amylose, magnesium stearate, surfactants, perfume oil, fatty acid monoglycerides, diglycerides and triglycerides, polyoxyethylated medium or long chain fatty acids such as ricinoleic acid, and polyoxyethylated fatty acid mono-, di, and triglycerides such as capric or caprilic acids, petroethral fatty acid esters, hydroxymethyl celluloses such as hydroxymethyl, hydroxyethyl, hydroxypropyl, hydroxypropyl acetate succinate, polyvinylpyrrolidone, crosspovidone and the like. The pharmaceutical compositions can be sterile and, if desired, mixed with auxiliary agents, like lubricants, preservatives, stabilizers, wetting agents, emulsifiers, salts for influencing osmotic pressure, buffers, colorings, flavoring and/or aromatic substances and the like which do not deleteriously react with the active compound.

If liquid dosage forms are considered for the present invention, these can include pharmaceutically acceptable emulsions, solutions, suspensions and syrups containing inert diluents commonly used in the art such as water. These dosage forms may contain e.g. microcrystalline cellulose for imparting bulk, alginic acid or sodium alginate as a suspending agent, methylcellulose as a viscosity enhancer and sweeteners/flavouring agents.

For parenteral application, particularly suitable vehicles consist of solutions, preferably oily or aqueous solutions, as well as suspensions, emulsions, or implants. Pharmaceutical formulations for parenteral administration are particularly preferred and include aqueous solutions of the compounds of formula (I) in water-soluble form. Additionally, suspensions of the compounds of formula (I) may be prepared as appropriate oily injection suspensions. Suitable lipophilic solvents or vehicles include fatty oils such as sesame oil, soybean oil, or tocopherols, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran.

Particularly preferred dosage forms are injectable preparations of a compound of formula (I). Thus, sterile injectable aqueous or oleaginous suspensions can for example be formulated according to the known art using suitable dispersing agents, wetting agents and/or suspending agents. A sterile injectable preparation can also be a sterile injectable solution or suspension or an emulsion in a non-toxic parenterally acceptable diluant or solvent. Among the acceptable vehicles and solvents that can be used are water and isotonic sodium chloride solution. Sterile oils are also conventionally used as solvent or suspending medium.

Suppositories for rectal administration of a compound of formula (I) can be prepared by e.g. mixing the compound with a suitable non-irritating excipient such as cocoa butter, synthetic triglycerides and polyethylene glycols which are solid at room temperature but liquid at rectal temperature such that they will melt in the rectum and release the compound according to formula (I) from said suppositories.

For administration by inhalation, the compounds according to the present invention may be conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Oral dosage forms may be liquid or solid and include e.g. tablets, troches, pills, capsules, powders, effervescent formulations, dragees and granules. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross-linked polyvinyl pyrrolidone (crosspovidone), agar, or alginic acid or a salt thereof such as sodium alginate. The oral dosage forms may be formulated to ensure an immediate release of the compound of formula (I) or a sustained release of the compound of formula (I).

A solid dosage form may comprise a film coating. For example, the inventive dosage form may be in the form of a so-called film tablet. A capsule of the invention may be a two-piece hard gelatin capsule, a two-piece hydroxypropylmethylcellulose capsule, a two-piece capsule made of vegetable or plant-based cellulose or a two-piece capsule made of polysaccharide.

The dosage form according to the invention may be formulated for topical application. Suitable pharmaceutical application forms for such an application may be a topical nasal spray, sublingual administration forms and controlled and/or sustained release skin patches. For buccal administration, the compositions may take the form of tablets or lozenges formulated in conventional manner.

The compositions may conveniently be presented in unit dosage forms and may be prepared by any of the methods well known in the art of pharmacy. The methods can include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories.

As regards human patients, the compound of formula (I) may be administered to a patient in an amount of about 0.001 mg to about 5000 mg per day, preferably of about 0.01 mg to about 100 mg per day, more preferably of about 0.1 mg to about 50 mg per day, which is the effective amount. The phrase “effective amount” means an amount of compound that, when administered to a mammal in need of such treatment, is sufficient to treat or prevent a particular disease or condition.

Furthermore, the pharmaceutical composition may also contain the compound of formula (I) as a prodrug such as an ester or amide thereof. A prodrug is any compound which is converted under physiological conditions or by solvolysis to any of the compounds of the invention. A prodrug may be inactive prior to administration but may be converted to an active compound of the invention in vivo.

Indications, for which the compounds of the present invention may be used

The compounds according to the present invention are preferably used for the treatment of a disease selected from the group consisting of cancer, Parkinson's disease, Huntington's disease, Alzheimer's disease, psychosis, stroke, extra pyramidal syndrome (in particular dystonia, akathisia, pseudoparkinsonism and tardive dyskinesia), attention deficit disorder (ADD), attention deficit hyperactivity disorder (ADHD), amyotrophic lateral sclerosis, cirrhosis, fibrosis, fatty liver, addictive behavior, dermal fibrosis (in particular dermal fibrosis in scleroderma), sleep disorders, AIDS, autoimmune diseases, infections, atherosclerosis and ischemia-reperfusion injury. The use for the treatment of cancer is particularly preferred.

More generally, the compounds according to the present invention can be used for the treatment of a disease selected from the group consisting of neurodegenerative, proliferative, inflammatory and infectious diseases, sickle cell disease, diabetic nephropathy, cognition and CNS disorders. The proliferative diseases include cancer.

Preferably, said cancer is selected from the group consisting of breast cancer, inflammatory breast cancer, ductal carcinoma, lobular carcinoma, colon cancer, pancreatic cancer, insulinomas, adenocarcinoma, ductal adenocarcinoma, adenosquamous carcinoma, acinar cell carcinoma, glucagonoma, skin cancer, melanoma, metastatic melanoma, lung cancer, small cell lung cancer, non-small cell lung cancer, squamous cell carcinoma, adenocarcinoma, large cell carcinoma, brain (gliomas), glioblastomas, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, head and neck, kidney, liver, melanoma, ovarian, pancreatic, adenocarcinoma, ductal adenocarcinoma, adenosquamous carcinoma, acinar cell carcinoma, glucagonoma, insulinoma, prostate, sarcoma, osteosarcoma, giant cell tumor of bone, thyroid, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, Immunoblastic large cell leukemia, mantle cell leukemia, multiple myeloma, megakaryoblastic leukemia, multiple myeloma, acute megakaryocyte leukemia, promyelocytic leukemia, erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), neuroendocrine cancers and testicular cancer.

More preferably, said cancer cancer is selected from the group consisting of brain (gliomas), glioblastomas, astrocytomas, glioblastoma multiforme, Bannayan-Zonana syndrome, Cowden disease, Lhermitte-Duclos disease, breast, colon, head and neck, kidney, lung, liver, melanoma, ovarian, pancreatic, adenocarcinoma, ductal adenocarcinoma, adenosquamous carcinoma, acinar cell carcinoma, glucagonoma, insulinoma, prostate, sarcoma and thyroid cancer.

Additionally, the compounds of the invention may be used to treat inflammation associated with autoimmune diseases or diseases resulting from inflammation including systemic lupus erythematosis, Addison's disease, autoimmune polyglandular disease (also known as autoimmune polyglandular syndrome), glomerulonephritis, rheumatoid arthritis scleroderma, chronic thyroiditis, Graves' disease, autoimmune gastritis, diabetes, autoimmune hemolytic anemia, glomerulonephritis, rheumatoid arthritis autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis, graft vs. host disease, asthma, bronchitis, acute pancreatitis, chronic pancreatitis and allergies of various types.

The compounds of the present invention may also be used to treat a neurodegenerative diseases including Alzheimer's disease (including early onset Alzheimer's disease), Parkinson's disease, amyotrophic lateral sclerosis, Huntington's disease, senile chorea, Sydenham's chorea, frontotemporal lobar dementia, spinocerebellar ataxia, dementia with Lewy bodies, cerebral ischemia or neurodegenerative disease caused by traumatic injury, glutamate neurotoxicity, hypoxia, peripheral neuropathy, including mononeuropathy, multiple mononeuropathy or polyneuropathy. Examples of peripheral neuropathy may be found in diabetes mellitus, Lyme disease or uremia, peripheral neuropathy caused by a toxic agent, demyelinating disease such as acute or chronic inflammatory polyneuropathy, leukodystrophies, or Guillain-Barre syndrome, multiple mononeuropathy secondary to a collagen vascular disorder (e.g. polyarteritis nodosa, SLE, Sjögren's syndrome), multiple mononeuropathy secondary to sarcoidosis, multiple mononeuropathy secondary to a metabolic disease (e.g. diabetes or amyloidosis), or multiple mononeuropathy secondary to an infectious disease (e.g Lyme disease or HIV infection).

Said pharmaceutical composition may comprise said compound as the only pharmaceutically active agent. It is to be understood that in connection with the medical uses of the invention, it can be preferred that the compounds according to the present invention are administered in combination with antibodies, radiotherapy, surgical therapy, immunotherapy, chemotherapy, toxin therapy, gene therapy, or any other therapy known to those of ordinary skill in the art for treatment of a particular disease. This is particularly relevant in connection with the treatment of cancer.

Preferably, the compounds of the present invention may be coadministered with an anti-neoplastic agent and/or an anti-neoplastic agent may be comprised in the pharmaceutical composition according to the present invention. The cancer treated by the combination of (i) a compound according to the present invention and (ii) an anti-neoplastic agent may be selected from one of the cancers listed above. It can further be preferred that said cancer is selected from the group consisting of colon, pancreatic cancer, breast cancer, prostate cancer, lung cancer including squamous non-small cell lung cancer (NSCLC) and non-squamous NSCLC, ovarian cancer, cervical cancer, renal cancer, cancer of the head and neck, lymphoma, leukemia, colorectal cancer, gastric cancer, melanoma, hepatocellular carcinoma, pancreatic carcinoma and a hematological malignancy.

An anti-neoplastic agent has activity versus a tumor and examples can be found in Cancer Principles and Practice of Oncology by V. T. Devita and S. Hellman (editors), 6th edition (Feb. 15, 2001), Lippincott Williams & Wilkins Publishers.

Typical anti-neoplastic agents useful in the present invention include chemotherapeutic agents, topoisomerase II inhibitors, antimetabolites, topoisomerase I inhibitors, hormones and hormonal analogues, signal transduction pathway inhibitors, non-receptor tyrosine kinase inhibitors, angiogenesis inhibitors, proapoptotic agents, cell cycle signaling inhibitors, proteasome inhibitors, inhibitors of cancer metabolism, and immunotherapeutic agents (such as STING pathway modulating compounds, TLR agonists and checkpoint inhibitors).

Examples for chemotherapeutic agents are anti-microtubule or anti-mitotic agents (such as paclitaxel), platinum coordination complexes (such as cisplatin), alkylating agents (such as cyclophosphamide) and antibiotic anti-neoplastics (such as doxorubicin).

It is widely known today that tumors can evade the immune system by suppressing the immune response. As discussed above, a strategy for doing so resides in the change of the microenvironment. Another (additional) strategy resides on the upregulation of receptors, which act (co-)inhibitory on the immune system (a negative “immune checkpoint” or “checkpoint”). Agents blocking or inhibiting these receptors (thus resulting in a block of the immunosuppressive signals of the tumor) are commonly referred to as “checkpoint inhibitors” and this reference is also used herein. The corresponding receptors targeted by such agents are PD-1, PD-L1, CTLA-4, IDO, KIR, TIM-3, LAG-3, CD39, CD73, ICOS, OX40, Tim-3, Vista, BTLA, TDO, and TIGIT and such agents are typically antibodies (including variants, such as e.g. fusion proteins or the like, thereof) but may also be macrocyclic inhibitors or the like.

A checkpoint inhibitor as described herein can in particular be an antibody selected from the group consisting of an anti-PD-1, anti-PD-L1, anti-CTLA-4, anti-IDO, anti-KIR, anti-TIM-3, anti-LAG-3, anti-CD39, anti-CD73, anti-ICOS, anti-OX40, anti-Tim-3, anti-Vista, anti-BTLA, anti-TDO, and anti-TIGIT-antibody. Specific examples are BMS-936559, MPDL3280A and MED14736 (anti-PD-1 antibodies), MK-3475 and pembrolizumab (anti-PD-1 antibodies) as well as ipilimumab (anti-CTLA-4 antibodies).

Preferably, the compounds of the present invention are administered in combination with antibodies. Preferred antibodies include anti-PD-1, anti-PD-L1, anti-CTLA-4, anti-IDO, anti-KIR, anti-TIM-3, anti-Vista, anti-TIGIT, anti-BTLA and anti-LAG3 antibody. Non-limiting examples are BMS-936559, MPDL3280A and MED14736 or avelumab (anti-PD-1 antibodies), MK-3475, pembrolizumab or pidilizumab (anti-PD-1 antibodies) as well as ipilimumab (anti-CTLA-4 antibodies). Preferably, the compounds of the present invention are administered in a pharmaceutical composition comprising one or more of adjuvants, inactivated or attenuated bacteria (e.g., inactivated or attenuated Listeria monocytogenes), modulators of innate immune activation, preferably agonists of Toll-like Receptors (TLRs, preferably TLR7 or TLR9 agonists, e.g. SM360320, AZD8848), (NOD)-like receptors (NLRs, preferably NOD2 agonist), retinoic acid inducible gene-based (RIG)-l-like receptors (RLRs), C-type lectin receptors (CLRs), or pathogenassociated molecular patterns (“PAMPs”), cytokines (not limiting examples e.g. IL-2, IL-12, IL-6), interferons (including, but not limited to IFN alpha, IFN beta, IFN gamma, IFN lambda) or chemotherapeutic agents. The medical use may further compromise administering at least one HBV vaccine, a nucleoside HBV inhibitor or any combination thereof (e.g. RECOMBIVAX HB, ENGERIX-B, GENEVAC-B).

Combination therapy may be achieved by use of a single pharmaceutical composition that includes both agents, or by administering two distinct compositions at the same time, wherein one composition includes a compound of the present invention, and the other includes the second agent(s).

The two therapies may be given in either order and may precede or follow the other treatment by intervals ranging from minutes to weeks. In embodiments where the other agents are applied separately, one would generally ensure that a significant period of time did not expire between the time of each delivery, such that the agents would still be able to exert an advantageously combined effect on the patient. In such instances, it is contemplated that one may administer both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. In some embodiments, the compound of the present invention is administered prior to administration of the distinct cancer treatment. In other embodiments, the distinct cancer treatment is administered prior to administration of the compound of the present invention.

The present invention is further illustrated by the following examples.

### EXAMPLES

Part 1: Synthesis

General

Microwave heating was done using a Biotage Emrys Initiator microwave or Microwave Reactor Anton Paar Monowave 450. Column chromatography was carried out using an Isco Rf200 d or an Interchim Puriflash 450. Solvent removal was carried out using either a Buchi rotary evaporator or a Genevac centrifugal evaporator. Preparative LC/MS was conducted using a Waters mass directed auto-purification system and a Waters 19×100 mm XBridge 5 micron C18 column under basic mobile phase conditions or an equivalent Waters CSH C18 column under acidic conditions. NMR spectra were recorded using a Bruker 300 MHz or 400 MHz spectrometer. Chemical shifts (6) are reported in ppm relative to the residual solvent signal (measurement range—6.4 kHz). 1H NMR data are reported as follows: chemical shift (multiplicity, coupling constants and number of hydrogens). Multiplicity is abbreviated as follows: s (singlet), d (doublet), t (triplet), q (quartet), m (multiplet), br (broad).

When the term “inerted” is used to describe a reactor (e.g., a reaction vessel, flask, glass reactor, and the like) it is meant that the air in the reactor has been replaced with an essentially moisture-free or dry, inert gas (such as nitrogen, argon, and the like).

### Preparative HPLC Conditions for the Purification of Target Compounds

Chromatography Conditions 1:

Prep HPLC Instrument: Waters 2545 pump with 2767 fraction collector
Column: Waters Xbridge C18 100 mm×19 mm, 5 μm particle size
MS Detector: Waters 3100 mass detector
UV detector: Waters 2489 dual wavelength UV detector
Flow Rate: 30 mL/min

### Example Gradient Time:

| Time(min) | B % |
|---|---|
| 0 | 20 |
| 1.5 | 20 |
| 6.5 | 40 |
| 6.55 | 95 |
| 8.5 | 95 |

### Representative Mobile Phase:

(1)
Mobile Phase: A: 0.1% formic acid in water
Mobile Phase: B: 0.1% formic acid in ACN
(2)
Mobile Phase: A: 0.1% NH4OH in water

### Mobile Phase: B: 0.1% NH4OH in ACN

### Chromatography Conditions 2:

### Prep HPLC Instrument: Shimadzu

### Column: Gemini-NX 5 μm C18 110 Å, 100*30 mm

### Detector: SPD-20A/20AV UV-VIS

Flow Rate: 30 mL/min

### Representative Mobile Phase:

(1)
Mobile Phase: A: 0.01% formic acid in water or TFA
Mobile Phase: B: 0.01% formic acid in ACN or TFA
(2)
Mobile Phase: A: 0.01% NH4OH in water

### Mobile Phase: B: 0.01% NH4OH in ACN

### Preparative SFC Conditions for the Purification of Target Compounds

### Chromatography Conditions:

### SFC Instrument: Thar SFC Prep Investigator (Waters)

Column: Chiral Technologies chiralpak IA 250 mm×10 mm, 5 μm particle size
ELS Detector: Waters 2424 detector
UV detector: Waters 2998 photodiode array detector, 254 nm
Flow Rate: 10 mL/min
Isocratic run: 40% isopropanol as a cosolvent
UPLC, HPLC and MS data provided in the examples described below were registered on:

### LC-MS Analyses on Bruker Amazon SL

Method name: Ic-ms1-2-ba

### Equipment:

MS Bruker Amazon SL
LC Dionex Ultimate 3000
HPLC with UV-Vis or DAD detector
column: Waters Acquity UPLC HSS C18, 50 mm×2.1 mm×1.8 μm

### Eluents:

(A) 0.1% formic acid in ACN
(B) 0.1% formic acid in water

### Analytical Method:

Autosampler:injection volume: 1 μL
Pump:

| Time | Flow | % |
|---|---|---|
| [min] | [ml/min] | B |
| 0.00 | 0.5 | 95 |
| 0.00 | 0.5 | 95 |
| 4.00 | 0.5 | 5 |
| 5.00 | 0.5 | 5 |
| 5.20 | 0.5 | 95 |
| 6.00 | 0.5 | 95 |

Column compartment:
   column temperature: 25° C.
   time of analysis: 6 min
Detector:
   wave length: 254, 230, 270, 280 nm

### LC-MS Analyses Bruker Amazon SL

Method name: BCM-30

### Equipment:

MS Bruker Amazon SL
LC Dionex Ultimate 3000
HPLC with UV-Vis or DAD detector
column: Waters Symmetry C18 3.9×150 mm 5 μm

### Eluents:

(A) 0.1% formic acid-water solution
(B) 0.1% formic acid-ACN solution

### Analytical Method:

Autosampler: injection volume: 3 μL
Pump:
flow: 1.2 ml/min

| Time | [%] |
|---|---|
| [min] | B |
| 0.0 | 20 |
| 20.0 | 80 |
| 22.0 | 80 |
| 22.5 | 95 |
| 25.0 | 95 |
| 25.3 | 20 |
| 30.0 | 20 |

Column compartment:
   column temperature: 25° C.
   time of analysis: 30 min
Detector:
   wave length: 254 nm

### LC-MS Analyses on Shimadzu:

Method name: Ic-ms1-2-ba

### Equipment:

Shimadzu LC-MS 2020
HPLC with UV-Vis or DAD detector
column: Waters Acquity UPLC HSS C18, 50 mm×2.1 mm×1.8 μm

### Eluents:

(A) 0.1% formic acid in ACN
(B) 0.1% formic acid in water

### Analytical Method:

Autosampler: injection volume: 1 μL
Pump:

| Time | Flow | % |
|---|---|---|
| [min] | [ml/min] | B |
| 0.00 | 0.5 | 95 |
| 0.00 | 0.5 | 95 |
| 4.00 | 0.5 | 5 |
| 5.00 | 0.5 | 5 |
| 5.20 | 0.5 | 95 |
| 6.00 | 0.5 | 95 |

Column compartment
   column temperature: 25° C.
   time of analysis: 6 min
Detector:
   wave length: 254, 230, 270, 280 nm

LC-MS Analyses on Corona Ultra:

Method name: BCM-30

### Equipment:

Corona ultra
LC Dionex Ultimate 3000
column: Waters Symmetry C18 3.9×150 mm 5 μm

### Eluents:

(A) 0.1% formic acid-water solution
(B) 0.1% formic acid-ACN solution

### Analytical Method:

Autosampler: injection volume: 3 μL
Pump:
flow: 1.2 ml/min

| Time | [%] |
|---|---|
| [min] | B |
| 0.0 | 20 |
| 20.0 | 80 |
| 22.0 | 80 |
| 22.5 | 95 |
| 25.0 | 95 |
| 25.3 | 20 |
| 30.0 | 20 |

The following abbreviations are used herein:

### ACN: Acetonitrile

### aq.: Aqueous

cAMP: Cyclic adenosine monophosphate
cod: cyclooctadiene

### Conc.: Concentrated

dba: dibenzylideneacetone

### DCM: Dichloromethane

### DCE: 1,2-Dichloroethane

DIPEA: N-ethyl-N-isopropylpropan-2-amine
DME: Dimethyl etherDMF: Dimethylformamide

### DMSO: Dimethylsulfoxide

ESI-MS: Electrospray ionization-mass spectrometry
Et₂O: Diethyl ether

### EtOH: Ethanol

EtOAc: Ethyl acetate

### Et₃N: Triethylamine

HATU: 1-[Bis(dimethylamino)methylene]-1H-1,2,3-triazolo[4,5-b]pyridinium 3-oxide hexafluorophosphate

### HTRF: Homogeneous Time Resolved Fluorescence

i-PrOH: Isopropanol
LCMS: Liquid chromatography-mass spectrometry

### MeOH: Methanol

### MW: Microwave

### NBS: N-bromosuccinimide

### NCS: N-chlorosuccinimide

### NECA: 5′-(N-Ethylcarboxamido)adenosine

### NIS: N-iodosuccinimide

NMR: Nuclear magnetic resonance
on or o.n.: overnight
prep-HPLC: Preparative high-performance liquid chromatography
prep-TLC: Preparative thin layer chromatography
Pd(amphos)Cl₂: Bis(di-tert-butyl(4-dimethylaminophenyl)phosphine)dichloropaladium(II)

### Pd(dppf)Cl₂: [1,1′-Bis(diphenylphosphino)ferrocene]dichloropalladium(II)

Pd(PPh₃)₄: Tetrakis(triphenylphosphine)palladium(0)
Pd Sphos G3: (2-Dicyclohexylphosphino-2′,6′-dimethoxybiphenyl) [2-(2′-amino-1,1′-biphenyl)]palladium(II) methanesulfonate
r.b.: round-bottom
RT or r.t.: Room temperature
[tBu-Py]₂: 4,4′-di-tert-butyl-2,2′-bipyridine

### TEA: Triethylamine

TFA: Trifluoroacetic acid

### THF: Tetrahydrofuran

TLC: Thin-layer chromatography
XPhos: 2-Dicyclohexylphosphino-2′,4′,6′-triisopropylbiphenyl

Materials: The following compounds are commercially available and/or can be prepared in a number of ways well known to one skilled in the art of organic synthesis. More specifically, disclosed compounds can be prepared using the reactions and techniques described herein. In the description of the synthetic methods described below, it is to be understood that all proposed reaction conditions, including choice of solvent, reaction atmosphere, reaction temperature, duration of the experiment, and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

### Procedure A1: Preparation of 2-methyl-4-(tetramethyl-1,3,2-dioxaborolan-2-yl)-6-(trifluoromethyl)pyridine

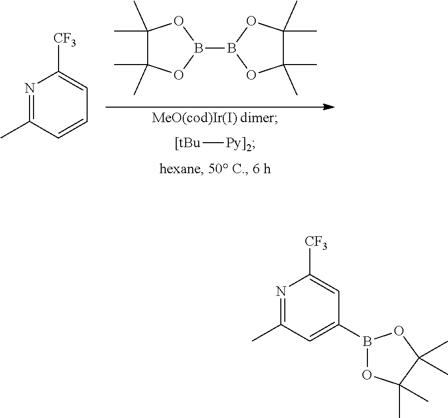

In a two-neck flask, equipped with stirring bar, condenser and a rubber septum, thoroughly purged with argon, were introduced methoxy(cyclooctadiene)iridium(I) dimer (21 mg, 0.03 mmol), 4,4′-di-tert-butyl-2,2′-bipyridine (17 mg, 0.06 mmol), and bis(pinacolato)diboron (2.05 g, 8.1 mmol). The flask was once more purged before adding hexane via syringe (15 mL). The resulting mixture was heated at 50° C. for 10 min until the appearance of a dark-red solution was observed. 2-trifluoromethyl-6-methyl pyridine (2.0 g, 12.4 mmol) was then added by syringe, and heating continued for a further 6 h. After cooling to room temperature, the crude reaction mixture was concentrated under reduced pressure. The resulting residue was purified by column chromatography, eluting with hexane/ethyl acetate mixture to afford the target compound 2-(trifluoromethyl)-4-(4,4,5,5-tetramethyl-1,3,2-dioxaborolan-2-yl)-6-methylpyridine (2.95 g, 83%) as a light brown thick oil. ESI-MS: 206.20 [M+H]+(boronic acid).

### Preparation of 2-chloro-6-methyl-4-(tetramethyl-1,3,2-dioxaborolan-2-yl)pyridine

The title compound was synthesized following the approach outlined in Procedure A1 substituting 2-chloro-6-methylpyridine for 2-trifluoromethyl-6-methyl pyridine to afford the title compound as a white solid (7.59 g, 29.94 mmol, 76%). ESI-MS: 172.00 [M+H]+. 1H NMR (300 MHz, CDCl₃) δ 7.49 (s, 1H), 7.42 (s, 1H), 2.55 (s, 3H), 1.36 (s, 12H).

### Procedure A2: Preparation of 5-(tetramethyl-1,3,2-dioxaborolan-2-yl)-1H-indazole

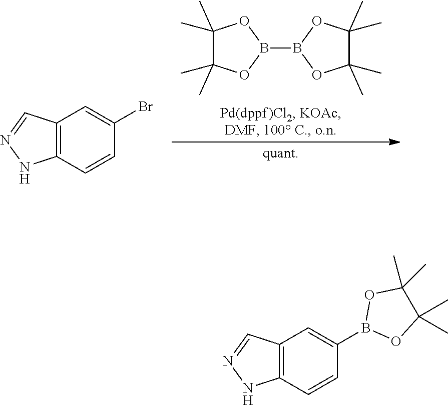

In a pressure tube, 5-bromo-1H-indazole (400 mg, 2 mmol), bis(pinacolato)diboron (773 mg, 3 mmol) and KOAc (598 mg, 6 mmol) were dissolved in 40 mL of dry DMF and sparged with argon for 10 min. Pd(dppf)Cl₂ (149 mg, 0.2 mmol) was added in one portion, and the reaction mixture was sparged with argon for additional 3 min. The pressure tube was capped and the reaction mixture was heated at 100° C. overnight. After full conversion (monitored by LCMS), the reaction mixture was filtered through Celite® and the filtrate was concentrated under reduced pressure. The residue was dissolved in EtOAc and co-evaporated with silica. Product was purified by column chromatography, eluting with hexane:EtOAc (0-50%) to afford the title product as a white solid (0.5 g, 2 mmol, quant.). ESI-MS: 245.1 [M+H]+. 1H NMR (300 MHz, DMSO-d6) δ 13.15 (s, 1H), 8.16 (s, 1H), 8.12 (s, 1H), 7.61 (dd, J=8.4, 1.1 Hz, 1H), 7.52 (dt, J=8.4, 1.0 Hz, 1H), 1.31 (s, 12H).

### Procedure B: Example 1: 4-{8-amino-6-phenylimidazo[1,2-a]pyrazin-5-yl}-2-chlorophenol

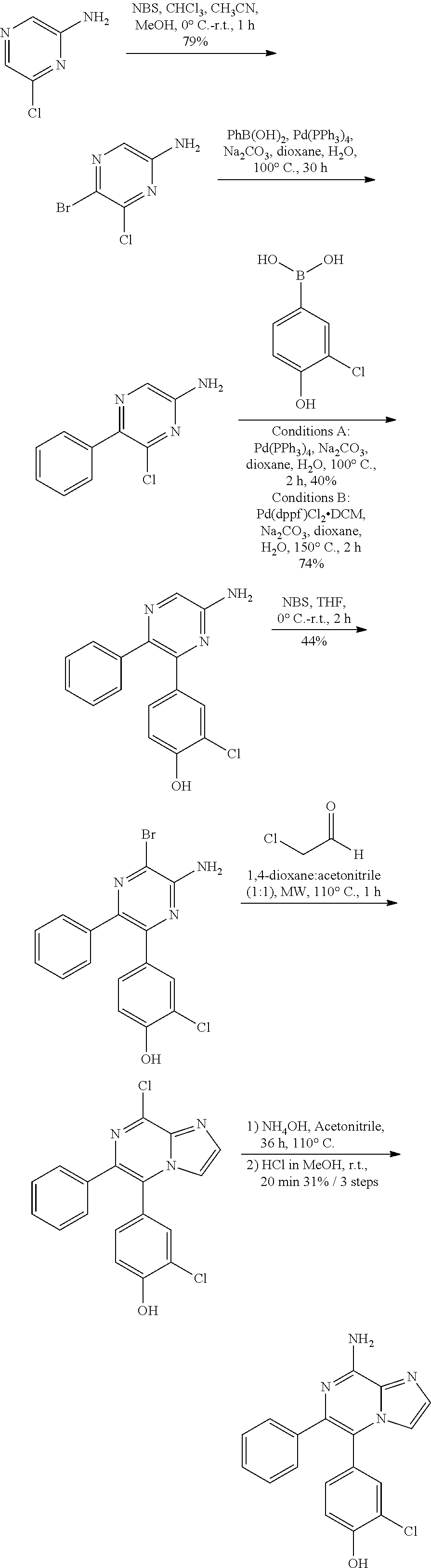

### a. 5-bromo-6-chloropyrazin-2-amine

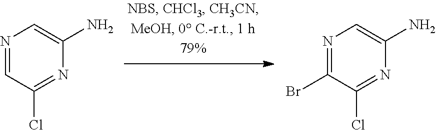 [SMILES: Nc1cncc(Cl)n1 ; Nc1cnc(Br)c(Cl)n1]

To a solution of 2-amino-6-chloropyrazine (5 g, 38.60 mmol) in a mixture of anhydrous chloroform (120 mL), anhydrous acetonitrile (12 mL) and anhydrous methanol (12 mL) was added slowly, at 0° C., NBS (7.56 g, 42.45 mmol, 1.1 eq.) and the mixture was warmed to room temperature and continuously stirred for 1 h. The excess solvent was removed in vacuo and the obtained crude material (light brown solid) was purified by column chromatography (Hexane/DCM/MeOH=50/50/0 then 0/100/0 then 0/95/5) to afford 5-bromo-6-chloropyrazin-2-amine (6.34 g, 30.42 mmol, 79%) as white crystals. ESI-MS: 209.90 [M+H]+. 1H NMR (300 MHz, CDCl3) δ 7.69 (s, 1H), 4.78 (br s, 2H).

### b. 6-chloro-5-phenylpyrazin-2-amine

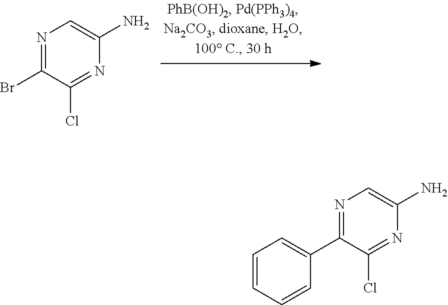 [SMILES: Nc1cnc(Br)c(Cl)n1 ; Nc1cnc(-c2ccccc2)c(Cl)n1]

In a pressure tube were mixed 5-bromo-6-chloropyrazin-2-amine (4.32 g, 20.73 mmol), phenyl boronic acid (2.78 g, 22.80 mmol, 1.1 equiv.) and Na2CO3 (4.39 g, 41.45 mmol, 2 equiv.) in a 4:1 mixture of 1,4-dioxane:water (50 mL). The reaction mixture was sparged with argon and Pd(PPh3)4 (1.20 g, 1.04 mmol, 5 mol %) was then added. The pressure tube was capped and the reaction mixture was heated at 100° C. overnight. After that time, the reaction mixture was cooled down to r.t., filtered and concentrated under reduced pressure. The obtained crude material was purified by flash chromatography on silica eluting with hexane:EtOAc=1:0-1:1 to lead to the title product as a white solid (2.11 g, 50%). ESI-MS: 206.05 [M+H]+. 1H NMR (300 MHz, DMSO-d6) δ 7.93 (s, 1H), 7.66-7.59 (m, 2H), 7.47-7.32 (m, 3H), 7.00 (br s, 2H).

### c. 4-(6-amino-3-phenylpyrazin-2-yl)-2-chlorophenol

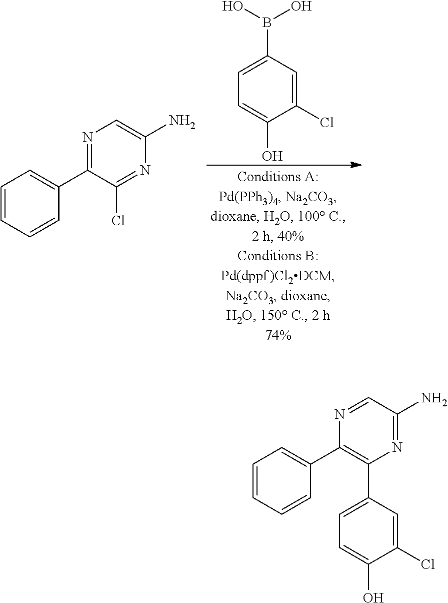 [SMILES: Nc1cnc(-c2ccccc2)c(Cl)n1 ; OB(O)c1ccc(O)c(Cl)c1 ; Nc1cnc(-c2ccccc2)c(-c2ccc(O)c(Cl)c2)n1]

In a pressure tube were mixed 5-phenyl-6-chloropyrazin-2-amine (100 mg, 0.49 mmol), (3-chloro-4-hydroxyphenyl)-boronic acid (100 mg, 0.58 mmol, 1.2 equiv.) and Na2CO3 (103 mg, 0.97 mmol, 2 equiv.) in a 4:1 mixture of 1,4-dioxane:water (2.5 mL). The reaction mixture was sparged with argon for 10 min. Conditions A or B described below were then applied. The reaction mixture was then cooled down to r.t., filtered through Celite® and rinsed with EtOAc. The organic solution was washed with water and brine and aqueous layer was extracted with EtOAc. Separated organic layer was dried over anhydrous sodium sulfate, filtered and concentrated under reduced pressure to afford the crude material as a brown residue. The obtained crude material was purified by flash chromatography on silica eluting with hexane and ethyl acetate to lead to the title product as an off-white solid (40% yield [Conditions A], 74% yield [Conditions B]). ESI-MS: 298.15 [M+H]+. 1H NMR (400 MHz, DMSO-d6) δ 10.35 (s, 1H), 7.89 (s, 1H), 7.33 (d, J=2.1 Hz, 1H), 7.30-7.20 (m, 5H), 6.98 (dd, J=8.4, 2.2 Hz, 1H), 6.79 (d, J=8.4 Hz, 1H), 6.59 (br s, 2H).
Conditions A: Pd(PPh3)4 (8 mol %) was then added and the reaction mixture was heated at 100° C. overnight.
Conditions B: Pd(dppf)Cl2.DCM (10 mol %) was then added and the reaction mixture was heated at 150° C. for 2 h.

### d. 4-(6-amino-5-bromo-3-phenylpyrazin-2-yl)-2-chlorophenol

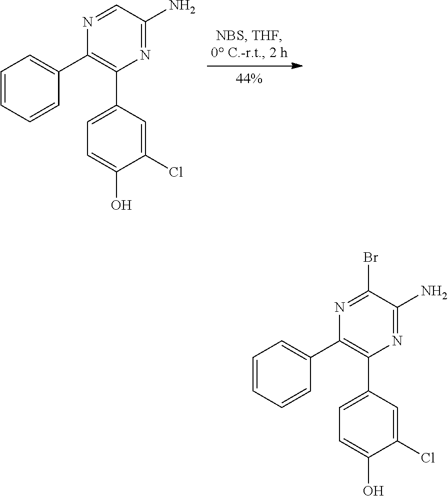 [SMILES: Nc1cnc(-c2ccccc2)c(-c2ccc(O)c(Cl)c2)n1 ; Nc1nc(-c2ccc(O)c(Cl)c2)c(-c2ccccc2)nc1Br]

To a solution of 4-(6-amino-3-phenylpyrazin-2-yl)-2-chlorophenol (0.408 g, 1.37 mmol) in THE (10 mL) cooled in an ice bath, was added N-bromosuccinimide (0.251 g, 1.41 mmol) portionwise. The reaction mixture was stirred at 0° C. for 2 h (TLC/LC-MS indicated completion of the reaction). Solvent was evaporated, residue was redissolved in EtOAc/water mixture. Organic layer was separated and aqueous layers was extracted with EtOAc. Combined organic layers were washed with brine, dried over anhydrous Na2SO4, filtered and evaporated. Crude material was purified by flash chromatography on silica eluting with DCM/EtOAc (0-20%) to afford the title product (205 mg, 0.54 mmol, 44%) as a brown solid. ESI-MS: 377.8 [M+H]+. 1H NMR (300 MHz, DMSO-d6) δ 10.43 (s, 1H), 7.36 (d, J=2.1 Hz, 1H), 7.33-7.23 (m, 5H), 6.99 (dd, J=8.4, 2.2 Hz, 1H), 6.87 (s, 2H), 6.81 (d, J=8.4 Hz, 1H).

### e. 2-chloro-4-{8-chloro-6-phenylimidazo[1,2-a]pyrazin-5-yl}phenol

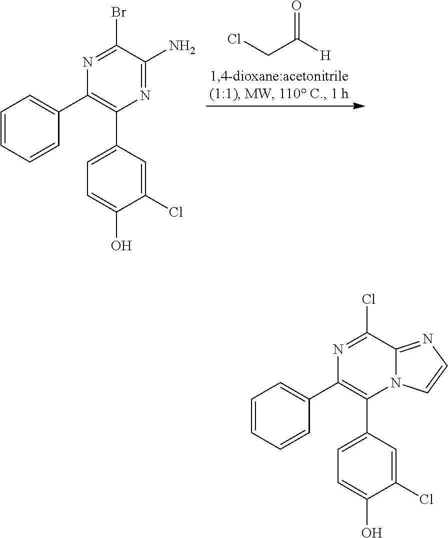

In a microwave reactor were mixed 4-(6-amino-5-bromo-3-phenylpyrazin-2-yl)-2-chlorophenol (250 mg, 0.66 mmol), chloroacetaldehyde (˜50% wt. in H2O, 1.27 mL, 10 mmol) in a 1:1 dioxane:acetonitrile mixture. The reaction mixture was then warmed to 110° C. under microwave irradiation for 1 h. After that time, the reaction mixture was cooled down to r.t. and concentrated under reduced pressure to lead to the title compound as a yellow residue that was used without further purification. ESI-MS: 355.90/357.85/359.85 [M+H]+.

### f. 4-{8-amino-6-phenylimidazo[1,2-a]pyrazin-5-yl}-2-chlorophenol

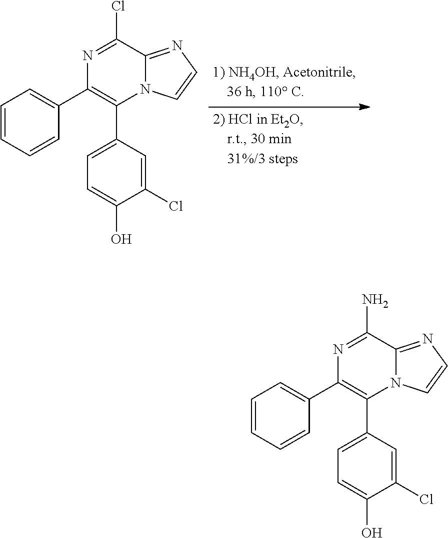 [SMILES: Oc1ccc(-c2c(-c3ccccc3)nc(Cl)c3nccn23)cc1Cl ; Nc1nc(-c2ccccc2)c(-c2ccc(O)c(Cl)c2)n2ccnc12] [0713] 1) In a pressure tube were mixed 2-chloro-4-{8-chloro-6-phenylimidazo[1,2-a]pyrazin-5-yl}phenol (1.18 g, 3.13 mmol) in acetonitrile (30 mL) then ammonium hydroxide solution (29%, 80 mL) was added and the reaction mixture was warmed to 110° C. and stirred overnight. After that time, additional amount of hydroxide solution (30 mL) was added and the reaction mixture was heated at 110° C. for 24 h more. After that time, the reaction mixture was cooled down to r.t. and concentrated under reduced pressure to afford a light brown residue. The crude material was purified by flash chromatography on silica eluting with DCM/EtOAc (0-20%) to afford the title as a pale yellow solid. ESI-MS: 336.90/338.90 [M+H]+.
2) The freebase product was dissolved in dry DCM (1 mL) then 2M HCl in diethylether (1 mL) was added. The reaction mixture was stirred for 1 h at r.t. (Formation of precipitate occurred during addition of ether solution). The suspension was concentrated under reduced pressure and lyophilized to obtained product as hydrochloride salt (yellow solid, 390 mg, 1.16 mmol, 31% over 3 steps). 1H NMR (400 MHz, DMSO-d6) δ 10.72 (s, 1H), 7.79 (s, 1H), 7.62 (s, 1H), 7.42-7.27 (m, 6H), 7.17 (d, J=8.0 Hz, 1H), 7.02 (d, J=8.4 Hz, 1H).

### Procedure C: Example 2: 4-[8-amino-6-(furan-2-yl)imidazo[1,2-a]pyrazin-5-yl]-2-chlorophenol

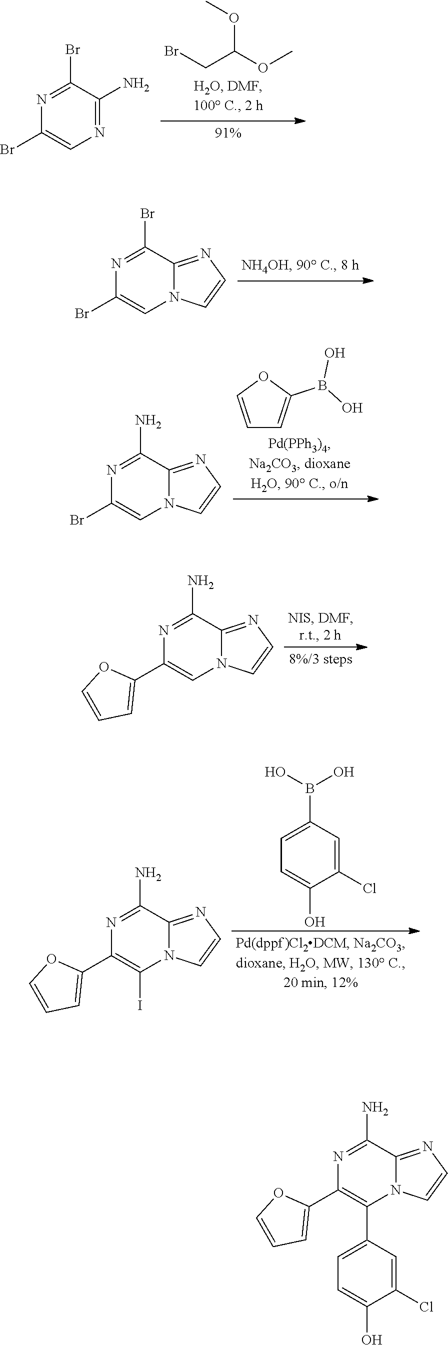

### a. 6,8-dibromoimidazo[1,2-a]pyrazine

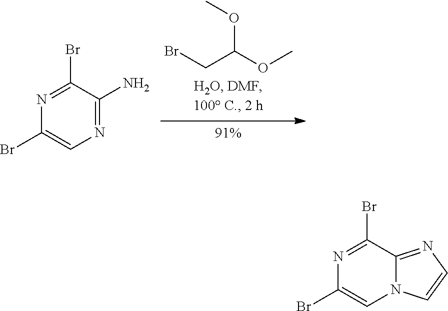

To a suspension of 2-amino-3,5-dibromopyrazine (2 g, 7.91 mmol) in water (25 mL) was added 2-bromo-1,1-dimethoxyethane (0.96 mL, 8.15 mmol, 1.03 equiv.) and the reaction mixture was heated at 100° C. for 2 h. The reaction mixture was then cooled down to r.t. and the resulting precipitate was collected by filtration and dried under reduced pressure overnight to afford the title product as a beige solid (2 g, 7.22 mmol, 91%) that was used without further purification. ESI-MS: 275.95/277.95/279.95 [M+H]+.

### b. 6-bromoimidazo[1,2-a]pyrazin-8-amine

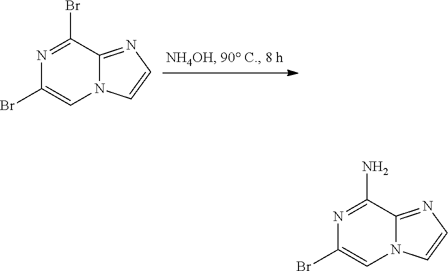 [SMILES: Brc1cn2ccnc2c(Br)n1 ; Nc1nc(Br)cn2ccnc12]

A solution of 6,8-dibromoimidazo[1,2-a]pyrazine (2 g, 7.22 mmol) in ammonium hydroxide solution (28-30%, 15 mL) was heated at 90° C. for 8 h then the reaction mixture was concentrated under reduced pressure to afford the title product as a light yellow solid (2.21 g) that was used without further purification. ESI-MS: 213.15/215.15 [M+H]+.

### c. 6-(furan-2-yl)imidazo[1,2-a]pyrazin-8-amine

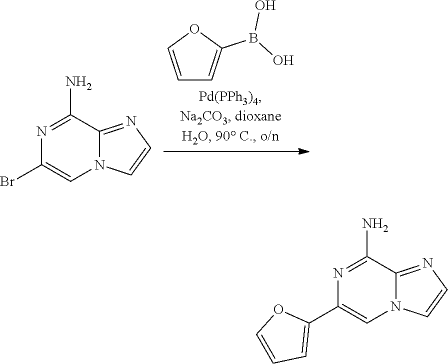 [SMILES: OB(O)c1ccco1 ; Nc1nc(Br)cn2ccnc12 ; Nc1nc(-c2ccco2)cn2ccnc12]

The title compound was synthesized following the approach outlined in Procedure B substituting 2-furanylboronic acid for phenylboronic acid and substituting 6-bromoimidazo[1,2-a]pyrazin-8-amine for 5-bromo-6-chloropyrazin-2-amine in step (b) to afford the title compound as an orange solid (620 mg, quant. yield). ESI-MS: 201.20 [M+H]+.

### d. 6-(furan-2-yl)-5-iodoimidazo[1,2-a]pyrazin-8-amine

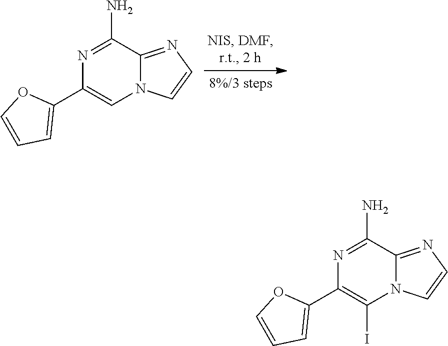 [SMILES: Nc1nc(-c2ccco2)cn2ccnc12 ; Nc1nc(-c2ccco2)c(I)n2ccnc12]

To a solution of 6-(furan-2-yl)imidazo[1,2-a]pyrazin-8-amine (0.2 g, 1 mmol) in anhydrous DMF (8 mL), was added N-iodosuccinimide (0.247 g, 1.1 mmol, 1.1 equiv.) in one portion. The reaction mixture was stirred at r.t. for 2 h then concentrated under reduced pressure and diluted in EtOAc and water. Aqueous layer was extracted with EtOAc and combined organic layers were dried over anhydrous Na2SO4, filtered and concentrated under reduced pressure to afford the crude material as brown residue. Both obtained regioisomers were separated by flash chromatography on silica eluting with Hexane/EtOAc (2/1-0/1) to afford the title product (25 mg, 0.08 mmol, 8%/3 steps) as a pale yellow solid. ESI-MS: 327.05 [M+H]+.

### e. 4-[8-amino-6-(furan-2-yl)imidazo[1,2-a]pyrazin-5-yl]-2-chlorophenol

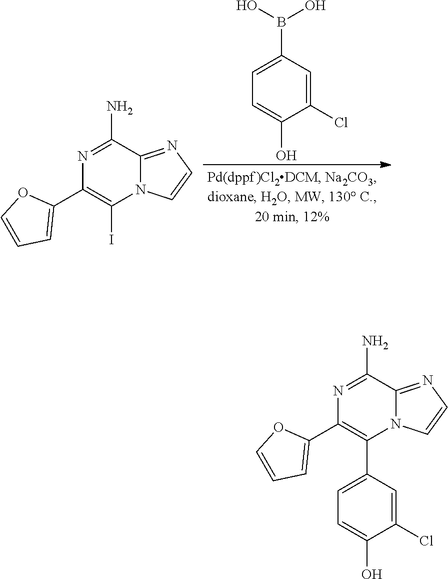 [SMILES: Nc1nc(-c2ccco2)c(I)n2ccnc12 ; OB(O)c1ccc(O)c(Cl)c1 ; Nc1nc(-c2ccco2)c(-c2ccc(O)c(Cl)c2)n2ccnc12]

The title compound was synthesized following the approach outlined in Procedure B, Conditions B substituting 6-(furan-2-yl)-5-iodoimidazo[1,2-a]pyrazin-8-amine for 6-chloro-5-phenylpyrazin-2-amine in step (c) and heating the reaction at 130° C. for 20 min under microwave irradiation to afford the title compound as a beige solid (6.2 mg, 12%). ESI-MS: 327.30 [M+H]+. 1H NMR (400 MHz, DMSO-d6) δ 10.66 (br s, 1H), 7.52 (d, J=1.7 Hz, 1H), 7.47 (d, J=1.2 Hz, 1H), 7.42 (d, J=2.0 Hz, 1H), 7.22 (d, J=1.3 Hz, 1H), 7.22-7.19 (dd, J=8.3, 2.1 Hz, 1H), 7.12 (d, J=8.3 Hz, 1H), 7.07 (br s, 2H), 6.41 (dd, J=3.3, 1.8 Hz, 1H), 6.13 (d, J=3.3 Hz, 1H).

### Example 3: 5-(2,6-dimethylpyridin-4-yl)-6-phenylimidazo[1,2-a]pyrazin-8-amine

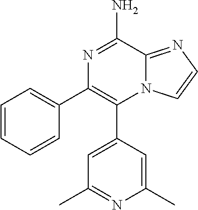 [SMILES: Cc1cc(-c2c(-c3ccccc3)nc(N)c3nccn23)cc(C)n1]

The title compound was synthesized following the approach outlined in Procedure B, Conditions A substituting 2,6-dimethylpyridine-4-boronic acid for (3-chloro-4-hydroxyphenyl)-boronic acid in step (c) and performing the step (e) in 1:1 ethanol:water mixture to afford the title compound as a pale yellow solid (18 mg, 17%). ESI-MS: 316.20 [M+H]+. 1H NMR (300 MHz, DMSO-d6) δ 7.52 (s, 1H), 7.45 (s, 1H), 7.33-7.15 (m, 7H), 7.02 (s, 2H), 2.36 (s, 6H).

### Example 4:5-[2-methyl-6-(trifluoromethyl)pyridin-4-yl]-6-phenylimidazo[1,2-a]pyrazin-8-amine

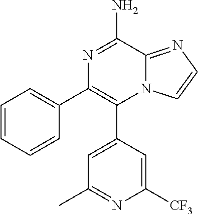 [SMILES: Cc1cc(-c2c(-c3ccccc3)nc(N)c3nccn23)cc(C(F)(F)F)n1]

The title compound was synthesized following the approach outlined in Procedure C substituting phenylboronic acid for 2-furanylboronic acid in step (c) and performing the reaction in 4:1 mixture of DME: H2O and performing the step (d) at 60° C. and substituting 2-methyl-4-(tetramethyl-1,3,2-dioxaborolan-2-yl)-6-(trifluoromethyl)pyridine (Procedure A1) for (3-chloro-4-hydroxyphenyl)-boronic acid in step (e) to afford the title compound as formate salt (beige solid, 9 mg, 27%). ESI-MS: 370.15 [M+H]+. 1H NMR (400 MHz, DMSO-d6) δ 7.70 (s, 1H), 7.67 (d, J=1.1 Hz, 1H), 7.58 (d, J=1.0 Hz, 1H), 7.50 (s, 1H), 7.33 (br s, 2H), 7.26 (s, 5H), 2.54 (s, 3H).

### Example 5: 4-[8-bromo-6-(furan-2-yl)imidazo[1,2-a]pyrazin-5-yl]-2-chlorophenol

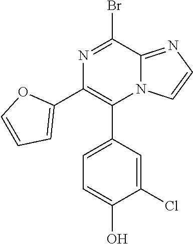 [SMILES: Oc1ccc(-c2c(-c3ccco3)nc(Br)c3nccn23)cc1Cl]

The title compound was synthesized following the approach outlined in Procedure B, Conditions A substituting 2-furanboronic acid for phenylboronic acid in step (b) and performing this step overnight and performing the reaction described in step (d) in 3:1 CHCl3:THF mixture for 15 h and substituting 2-bromo-1,1-dimethoxyethane for chloroacetaldehyde in step (e) and without need of step (f) to afford the title compound as pale yellow solid (8 mg, 7%). ESI-MS: 391.4 [M+H]+. 1H NMR (300 MHz, CD3OD) 9.05 (s, 1H), 7.80 (s, 1H), 7.62-7.56 (m, 1H), 7.56-7.50 (m, 1H), 7.33-7.27 (m, 1H), 7.22-7.15 (m, 1H), 6.46-6.36 (m, 2H).

### Example 6: 4-[8-amino-6-(4-fluorophenyl)imidazo[1,2-a]pyrazin-5-yl]-2-chlorophenol

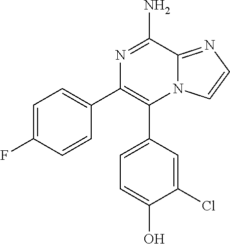 [SMILES: Nc1nc(-c2ccc(F)cc2)c(-c2ccc(O)c(Cl)c2)n2ccnc12]

The title compound was synthesized following the approach outlined in Procedure B, Conditions B substituting 4-fluorophenylboronic acid for phenylboronic acid in step (b) and heating the reaction for 6.5 h to afford the title compound as hydrochloride salt (pale yellow solid, 20 mg, 34%). ESI-MS: 355.40 [M+H]+. 1H NMR (300 MHz, DMSO-d6) δ 10.83 (br s, 1H), 8.92 (br s, 2H), 7.86 (s, 1H), 7.66 (s, 1H), 7.38 (m, 3H), 7.30-7.12 (m, 3H), 7.04 (d, J=8.4 Hz, 1H).

### Example 7:6-(4-fluorophenyl)-5-[2-methyl-6-(trifluoromethyl)pyridin-4-yl]imidazo[1,2-a]pyrazin-8-amine

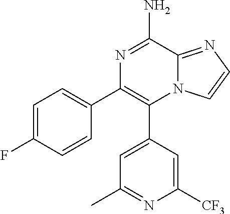 [SMILES: Cc1cc(-c2c(-c3ccc(F)cc3)nc(N)c3nccn23)cc(C(F)(F)F)n1]

The title compound was synthesized following the approach outlined in Procedure B, Conditions B substituting 4-fluorophenylboronic acid for phenylboronic acid in step (b) and heating the reaction for 6.5 h and substituting 2-methyl-4-(tetramethyl-1,3,2-dioxaborolan-2-yl)-6-(trifluoromethyl)pyridine (Procedure A1) for (3-chloro-4-hydroxyphenyl)-boronic acid in step (c) and substituting 2-bromo-1,1-dimethoxyethane for 2-chloroacetaldehyde in step (e) and performing this step in 10:1 ethanol:water to afford the title compound as hydrochloride salt (white solid, 7 mg, 14%). ESI-MS: 388.30 [M+H]+. 1H NMR (300 MHz, CDCl3) δ 7.80 (s, 1H), 7.53-7.43 (m, 2H), 7.42-7.25 (m, 5H), 7.15-6.99 (m, 2H), 2.67 (s, 3H).

### Example 8: 5-(2,6-dimethylpyridin-4-yl)-6-(4-fluorophenyl)imidazo[1,2-a]pyrazin-8-amine

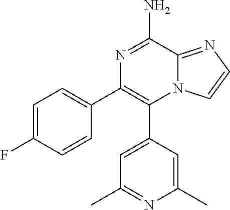 [SMILES: Cc1cc(-c2c(-c3ccc(F)cc3)nc(N)c3nccn23)cc(C)n1]

The title compound was synthesized following the approach outlined in Procedure B, Conditions A substituting 4-fluorophenylboronic acid for phenylboronic acid in step (b) and heating the reaction for 6.5 h and substituting 2,6-dimethylpyridinyl-4-boronic acid for (3-chloro-4-hydroxyphenyl)-boronic acid in step (c) and performing the reaction at 150° C. and substituting 2-bromo-1,1-dimethoxyethane for 2-chloroacetaldehyde in step (e) and performing this step in acetonitrile (containing 5% v/v water) and performing the step (f) in 1,4-dioxane:methanol (1.5:1) and heating the reaction at 80° C. overnight to afford the title compound as hydrochloride salt (pale yellow solid, 6 mg, 9%). ESI-MS: 334.30 [M+H]+. 1H NMR (300 MHz, CD3OD) δ 7.88-7.83 (m, 2H), 7.76-7.72 (m, 2H), 7.52-7.45 (m, 2H), 7.18 (t, J=8.7 Hz, 2H), 3.35 (s, 3H), 2.71 (s, 3H).

### Example 9:6-(3-fluorophenyl)-5-[2-methyl-6-(trifluoromethyl)pyridin-4-yl]imidazo[1,2-a]pyrazin-8-amine

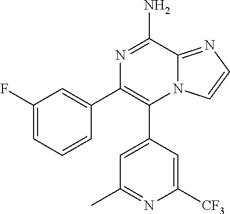 [SMILES: Cc1cc(-c2c(-c3cccc(F)c3)nc(N)c3nccn23)cc(C(F)(F)F)n1]

The title compound was synthesized following the approach outlined in Procedure B, Conditions B substituting 3-fluorophenylboronic acid for phenylboronic acid in step (b) and heating the reaction overnight and substituting 2-methyl-4-(tetramethyl-1,3,2-dioxaborolan-2-yl)-6-(trifluoromethyl)pyridine (Procedure A1) for (3-chloro-4-hydroxyphenyl)-boronic acid in step (c) and heating the reaction in step (e) in absolute ethanol for 2 h and heating the reaction in step (f) at 100° C. overnight to afford the title compound as hydrochloride salt (white solid, 28 mg, 29%). ESI-MS: 389.30 [M+H]+. 1H NMR (300 MHz, CDCl3) δ 7.76 (s, 1H), 7.41 (s, 2H), 7.35-7.25 (m, 2H), 7.12-6.95 (m, 3H), 3.22 (br s, 2H), 2.59 (s, 3H).

### Example 10:3-{8-amino-5-[2-methyl-6-(trifluoromethyl)pyridin-4-yl]imidazo[1,2-a]pyrazin-6-yl}benzonitrile

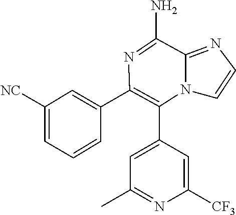 [SMILES: Cc1cc(-c2c(-c3cccc(C#N)c3)nc(N)c3nccn23)cc(C(F)(F)F)n1]

The title compound was synthesized following the approach outlined in Procedure B, Conditions B substituting 3-cyanophenylboronic acid for phenylboronic acid in step (b) and performing the reaction for 6.5 h and substituting 2-methyl-4-(tetramethyl-1,3,2-dioxaboroan-2-yl)-6-(trifluoromethyl)pyridine for (3-chloro-4-hydroxyphenyl)-boronic acid in step (c) and performing the reaction in step (d) overnight and performing the reaction in step (f) at 100° C. under microwave irradiation for 1.5 h to afford the title compound as hydrochloride salt (beige solid, 15 mg, 18%). ESI-MS: 395.30 [M+H]+. 1H NMR (300 MHz, CD3CN) δ 7.80 (d, J=1.4 Hz, 1H), 7.74 (dt, J=7.6, 1.5 Hz, 1H), 7.75-7.67 (m, 1H), 7.60 (d, J=1.4 Hz, 1H), 7.57 (dt, J=7.9, 1.5 Hz, 1H), 7.52-7.44 (m, 3H), 2.55 (s, 3H).

### Example 11: 3-[8-amino-5-(3-chloro-4-hydroxyphenyl)imidazo[1,2-a]pyrazin-6-yl]benzonitrile

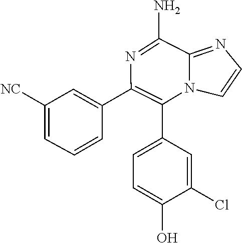 [SMILES: N#Cc1cccc(-c2nc(N)c3nccn3c2-c2ccc(O)c(Cl)c2)c1]

The title compound was synthesized following the approach outlined in Procedure B, Conditions B substituting 3-cyanophenylboronic acid for phenylboronic acid in step (b) and performing the reaction for 6.5 h and performing the reaction in step (e) at 100° C. under microwave irradiation for 45 min and performing the reaction in step (f) at 90° C. overnight to afford the title compound as hydrochloride salt (pale yellow solid, 15 mg, 15%). ESI-MS: 362.50 [M+H]+. 1H NMR (300 MHz, CD3OD) δ 7.81-7.76 (m, 2H), 7.75-7.69 (m, 1H), 7.64-7.58 (m, 2H), 7.50 (d, J=7.8 Hz, 1H), 7.35 (d, J=2.1 Hz, 1H), 7.14 (dd, J=8.4, 2.2 Hz, 1H), 6.94 (d, J=8.4 Hz, 1H).

### Example 12: 4-[8-amino-6-(3-fluorophenyl)imidazo[1,2-a]pyrazin-5-yl]-2-chlorophenol

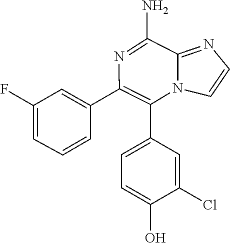 [SMILES: Nc1nc(-c2cccc(F)c2)c(-c2ccc(O)c(Cl)c2)n2ccnc12]

The title compound was synthesized following the approach outlined in Procedure B, Conditions B substituting 3-fluorophenylboronic acid for phenylboronic acid in step (b) and performing the reaction overnight and performing the reaction in step (f) at 110° C. under microwave irradiation for 1 h then using regular heating (oil bath) overnight to afford the title compound as hydrochloride salt (white solid, 30 mg, 18%). ESI-MS: 355.50 [M+H]+. 1H NMR (300 MHz, DMSO-d6) δ 10.79 (s, 1H), 8.75 (br s, 2H), 7.86 (d, J=1.3 Hz, 1H), 7.66 (d, J=1.3 Hz, 1H), 7.44-7.34 (m, 1H), 7.41 (d, J=2.1 Hz, 1H), 7.24-7.11 (m, 4H), 7.05 (d, J=8.4 Hz, 1H).

### Example 13: 4-[8-amino-2-(3-nitrophenyl)-6-phenylimidazo[1,2-a]pyrazin-5-yl]-2-chlorophenol

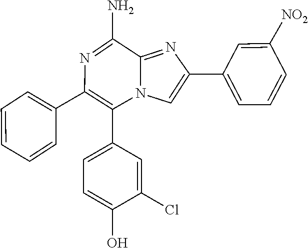 [SMILES: Nc1nc(-c2ccccc2)c(-c2ccc(O)c(Cl)c2)n2cc(-c3cccc([N+](=O)[O-])c3)nc12]

The title compound was synthesized following the approach outlined in Procedure B, Conditions B substituting 2-bromo-3′-nitroacetophenone for chloroacetaldehyde in step (e) and heating this reaction at 120° C. for 3 days to afford the title compound as hydrochloride salt (yellow solid, 7 mg, 11%). ESI-MS: 458.6 [M+H]+. 1H NMR (400 MHz, DMSO-d6) δ 10.68 (s, 1H), 8.93 (t, J=2.0 Hz, 1H), 8.55-8.48 (m, 1H), 8.34 (s, 1H), 8.30-8.10 (br s, 2H), 8.19 (dd, J=8.3, 2.3 Hz, 1H), 7.72 (t, J=8.0 Hz, 1H), 7.39 (d, J=2.1 Hz, 1H), 7.37-7.27 (m, 5H), 7.20 (dd, J=8.3, 2.1 Hz, 1H), 7.05 (d, J=8.4 Hz, 1H).

### Procedure D: Example 14: 5-(1H-indazol-5-yl)-6-phenylimidazo[1,2-a]pyrazin-8-amine

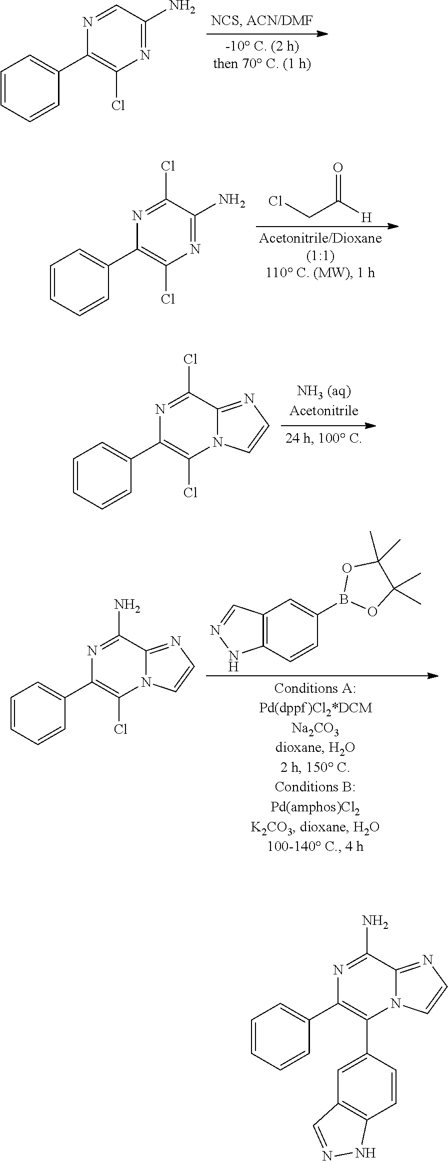

### a. 3,6-dichloro-5-phenylpyrazin-2-amine

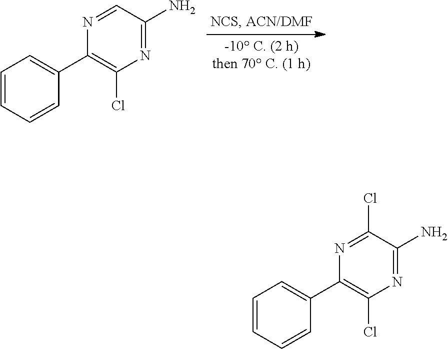 [SMILES: Nc1cnc(-c2ccccc2)c(Cl)n1 ; Nc1nc(Cl)c(-c2ccccc2)nc1Cl]

N-chlorosuccinimide (0.457 g, 3.42 mmol) was added in 3 portions to the solution of 6-chloro-5-phenylpyrazin-2-amine [Procedure B step (b), (0.670 g, 3.26 mmol)] in CH₃CN (10 mL) and DMF (3 mL) cooled in an NaCl/ice bath. The reaction mixture was allowed to warm to r.t. then was heated at 70° C. for 1 h. TLC indicated completion of the reaction. Solvent was evaporated, residue was dissolved in EtOAc/water mixture. Organic layer was separated and aqueous layers was extracted with EtOAc (1×). Combined organic layers were washed with brine, dried over anhydrous Na2SO4, filtered and evaporated. Crude product was purified by flash chromatography on silica eluting with hexane/EtOAc (0-20%) to afford the title product (579 mg, 82%) as a light yellow solid. ESI-MS: 239.9/241.90/243.90 [M+H]+. 1H NMR (300 MHz, DMSO-d6) δ 7.66-7.59 (m, 2H), 7.50-7.39 (m, 3H), 7.37 (br s, 2H).

### b. 5,8-dichloro-6-phenylimidazo[1,2-a]pyrazine

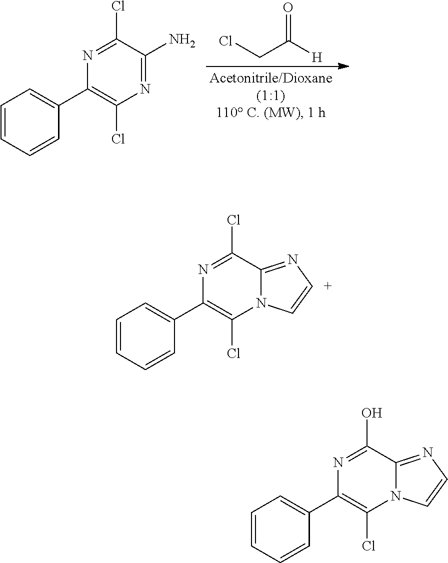

A MW vial (10-20 ml) was charged with 3,6-dichloro-5-phenylpyrazin-2-amine (0.500 g, 2.08 mmol) in acetonitrile/dioxane mixture (6/6 mL), then chloroacetaldehyde 50% in water (2.64 mL, 20.83 mmol) was added. The resulting mixture was heated at 110° C. under microwave irradiation for 1 h. LC-MS showed remaining SM. RM was heated in a MW at 110° C. for additional 1 h. TLC indicated completion of the reaction. The reaction mixture was concentrated and purified by flash chromatography on silica eluting with hexane/EtOAc 0-50% to afford the title product (411 mg, 75%) as thick brown oil. ESI-MS: 264.05/266.00/268.00 [M+H]+.

### c. 5-chloro-6-phenylimidazo[1,2-a]pyrazin-8-amine

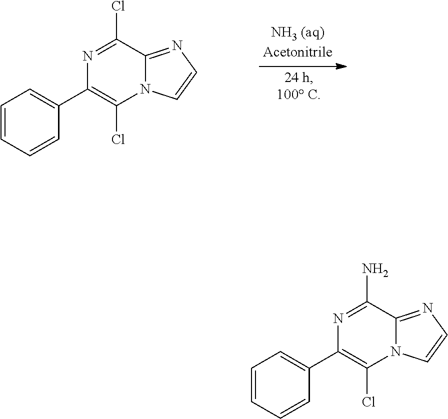 [SMILES: Clc1nc(-c2ccccc2)c(Cl)n2ccnc12 ; Nc1nc(-c2ccccc2)c(Cl)n2ccnc12]

A pressure tube was charged with 5,8-dichloro-6-phenylimidazo[1,2-a]pyrazine (0.411 g, 1.56 mmol) in acetonitrile (3 mL), then ammonia solution in water (8 mL) was added. The resulting mixture was heated for 24 h at 100° C. TLC indicated completion of the reaction. The reaction mixture was concentrated and purified by flash chromatography on silica eluting with hexane/EtOAc 0-60% to afford the title product (112 mg, 29%) as a brown foam. ESI-MS: 244.95/246.85 [M+H]+. 1H NMR (300 MHz, DMSO-d6) δ 8.04 (d, J=1.2 Hz, 1H), 7.70-7.65 (m, 3H), 7.50-7.40 (m, 3H).

### d. 5-(1H-indazol-5-yl)-6-phenylimidazo[1,2-a]pyrazin-8-amine

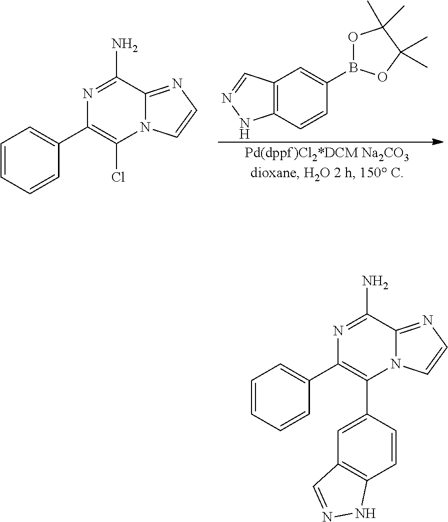

Conditions A: A pressure tube was charged with 5-chloro-6-phenylimidazo[1,2-a]pyrazin-8-amine (0.060 g, 0.25 mmol), 5-(tetramethyl-1,3,2-dioxaborolan-2-yl)-1H-indazole (0.072 g, 0.29 mmol), sodium carbonate (0.052 g, 0.49 mmol) and mixture of 1,4-dioxane and water 4:1 (2.5 mL). This mixture was then sparged with argon under sonication for a few minutes, subsequently Pd(dppf)Cl2*DCM was added (0.020 g, 0.02 mmol), the reaction mixture was sparged with argon shortly and the vessel was capped. The reaction mixture was heated at 150° C. for 2 h. LC-MS indicated completion of the reaction. The reaction mixture was filtered through Celite®, and the filtrate was concentrated. Crude product was purified by flash chromatography on silica eluting with DCM/MeOH 0-5%. Additional purification by RP-HPLC (formic acid) was performed to afford the title product as freebase. Obtained product was then suspended in a small volume of methanol and 2M HCl solution in diethyl ether (0.1 mL) was added. The resulting solution was stirred for 1 h at r.t. then concentrated under reduced pressure and finally lyophilized to afford the title product as hydrochloride salt (18 mg, 20%) as a yellow solid. ESI-MS: 327.05 [M+H]+. 1H NMR (400 MHz, DMSO-d6) δ 9.35-8.6 (br s, 1H), 8.20 (s, 1H), 7.89-7.82 (m, 2H), 7.65-7.55 (m, 2H), 7.38-7.33 (m, 3H), 7.32-7.26 (m, 3H), 5.05-4.15 (br s, 2H).

### Example 15: 4-{8-amino-6-phenylimidazo[1,2-a]pyrazin-5-yl}-2,6-dichlorophenol

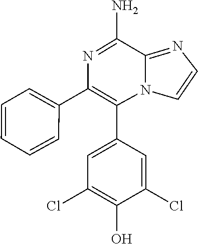 [SMILES: Nc1nc(-c2ccccc2)c(-c2cc(Cl)c(O)c(Cl)c2)n2ccnc12]

The title compound was synthesized following the approach outlined in Procedure B, Conditions B heating the reaction in step (b) overnight and substituting 3,5-dichloro-4-methoxyphenylboronic acid for (3-chloro-4-hydroxyphenyl)-boronic acid in step (c) and performing the reaction in step (f) at 110° C. under microwave irradiation for 1 h to afford the title compound as hydrochloride salt (white solid, 35 mg, 22%). ESI-MS: 370.85/372.85 [M+H]+. 1H NMR (300 MHz, DMSO-d6) δ 10.69 (br s, 1H), 8.85 (br s, 2H), 7.84 (d, J=1.3 Hz, 1H), 7.75 (d, J=1.3 Hz, 1H), 7.43 (s, 2H), 7.37 (s, 5H).

### Example 16: 4-{8-amino-2-cyclohexyl-6-phenylimidazo[1,2-a]pyrazin-5-yl}-2-chlorophenol

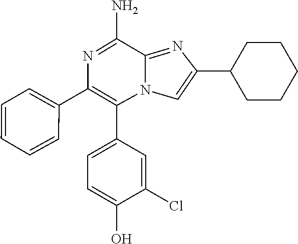 [SMILES: Nc1nc(-c2ccccc2)c(-c2ccc(O)c(Cl)c2)n2cc(C3CCCCC3)nc12]

The title compound was synthesized following the approach outlined in Procedure B, Conditions B heating the reaction in step (b) overnight and substituting 2-bromo-1-cyclohexylethan-1-one for chloroacetaldehyde in step (e) and heating this reaction at 90° C. for 64 h and performing the reaction in step (f) in 1,4-dioxane at 110° C. for 1 week to afford the title compound as hydrochloride salt (pale yellow solid, 9 mg, 18%). ESI-MS: 419.00/420.95 [M+H]+. 1H NMR (300 MHz, DMSO-d6) δ 10.67 (br s, 1H), 7.52-7.37 (m, 2H), 7.35-7.18 (m, 4H), 7.17-6.88 (m, 3H), 3.27-3.07 (m, 1H), 2.80-2.61 (m, 1H), 2.33-2.21 (m, 1H), 2.05-1.84 (m, 2H), 1.82-1.59 (m, 2H), 1.55-0.66 (m, 4H).

### Example 17: 4-{8-amino-6-phenylimidazo[1,2-a]pyrazin-5-yl}-2-bromo-6-chlorophenol

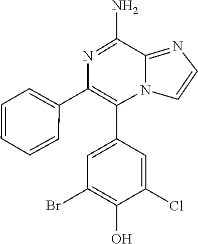 [SMILES: Nc1nc(-c2ccccc2)c(-c2cc(Cl)c(O)c(Br)c2)n2ccnc12]

The title compound was synthesized following the approach outlined in Procedure B, Conditions B performing the reaction in step (d) from 0° C. to r.t. overnight. 4-{8-amino-6-phenylimidazo[1,2-a]pyrazin-5-yl}-2-bromo-6-chlorophenol was obtained as minor product (pale yellow solid, 12%) and performing the reaction in step (f) at 90° C. overnight to afford the title compound as hydrochloride salt (yellow solid, 83 mg, 45%). ESI-MS: 417.1 [M+H]+. 1H NMR (300 MHz, DMSO-d6) δ 10.66 (br s, 1H), 9.21 (br s, 2H), 7.91 (s, 1H), 7.80 (s, 1H), 7.57 (s, 1H), 7.50 (s, 1H), 7.39 (s, 5H).

### Example 18: 4-{8-amino-6-[4-(trifluoromethyl)phenyl]imidazo[1,2-a]pyrazin-5-yl}-2-chlorophenol

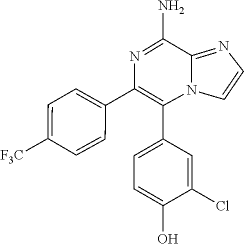 [SMILES: Nc1nc(-c2ccc(C(F)(F)F)cc2)c(-c2ccc(O)c(Cl)c2)n2ccnc12]

The title compound was synthesized following the approach outlined in Procedure B, Conditions A in step (c) and substituting 4-trifluoromethylphenylboronic acid for phenyl boronic acid in step (b) and performing the reaction in step (c) at 130° C. for 3 h and performing the reaction in step (e) at 100° C. for 45 min under microwave irradiation and performing the reaction in step (f) at 100° C. overnight to afford the title compound as hydrochloride salt (light beige solid, 45 mg, 24%). ESI-MS: 405.50 [M+H]+. 1H NMR (300 MHz, DMSO-d6) δ 10.89 (s, 1H), 8.92 (br s, 2H), 7.94 (s, 1H), 7.75 (d, J=8.3 Hz, 2H), 7.71 (d, J=1.2 Hz, 1H), 7.56 (d, J=8.1 Hz, 2H), 7.43 (d, J=2.0 Hz, 1H), 7.18 (dd, J=8.4, 2.0 Hz, 1H), 7.08 (d, J=8.4 Hz, 1H).

### Example 19: 3-[8-amino-5-(3-chloro-4-hydroxyphenyl)-6-phenylimidazo[1,2-a]pyrazin-2-yl]benzonitrile

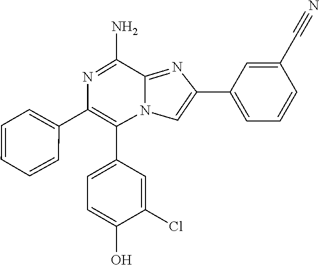 [SMILES: N#Cc1cccc(-c2cn3c(-c4ccc(O)c(Cl)c4)c(-c4ccccc4)nc(N)c3n2)c1]

The title compound was synthesized following the approach outlined in Procedure B, Conditions B in step (c) and substituting 3-(2-bromoacetyl)benzonitrile for 2-chloroacetaldehyde in step (e) and performing this step at 120° C. for 3 days and performing the reaction in step (f) at 100° C. for 48 h to lead to the title product (8 mg, 0.02 mmol, 5%) as a light yellow solid. ESI-MS: 438.7 [M+H]+. 1H NMR (300 MHz, DMSO-d6) δ 10.92 (s, 1H), 8.51 (t, J=1.7 Hz, 1H), 8.38 (dt, J=7.9, 1.5 Hz, 1H), 8.12 (s, 1H), 7.75 (dt, J=7.7, 1.4 Hz, 1H), 7.62 (t, J=7.8 Hz, 1H), 7.37-7.28 (m, 3H), 7.27-7.13 (m, 4H), 7.11 (br s, 2H), 7.03 (d, J=8.3 Hz, 1H).

### Procedure E: Example 20: 4-[8-amino-5-(3-chloro-4-hydroxyphenyl)imidazo[1,2-a]pyrazin-6-yl]benzonitrile

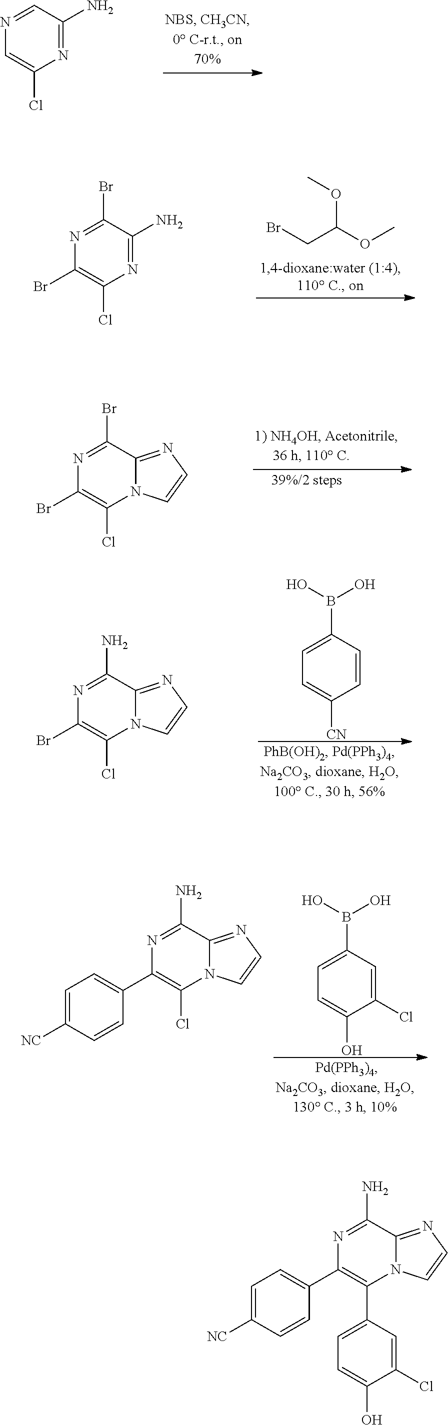

### a. 3,5-dibromo-6-chloropyrazin-2-amine

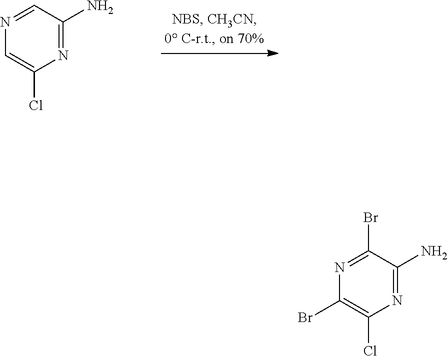 [SMILES: Nc1cncc(Cl)n1 ; Nc1nc(Cl)c(Br)nc1Br]

To a solution of 2-amino-6-chloropyrazine (1 g, 7.72 mmol) in anhydrous acetonitrile (10 mL) was gradually added NBS (4.12 g, 23.16 mmol, 3 equiv.) at 0° C. The reaction mixture was slowly warmed up to r.t. and stirred overnight then diluted with water and extracted with Et2O. Combined organic layers were washed with water and brine, then dried over anhydrous sodium sulfate, filtered and concentrated under reduced pressure. The crude material was purified by flash chromatography on silica eluting with Hexane/EtOAc (1:1) to give the title compound (1.55 g, 5.39 mmol, 70%) as a pale yellow solid. ESI-MS: 287.90 [M+H]+.

### b. 6,8-dibromo-5-chloroimidazo[1,2-a]pyrazine

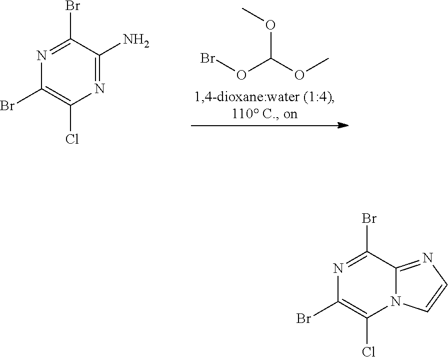

To a suspension of 3,5-dibromo-6-chloropyrazin-2-amine (0.55 g, 1.91 mmol) in a 4:1 mixture of water:1,4-dioxane (10 mL) was added 2-bromo-1,1-dimethoxyethane (0.34 mL, 2.87 mmol, 1.5 equiv.) and the reaction mixture was refluxed overnight. After that time, the reaction mixture was concentrated under reduced pressure to afford the title product as a beige powder that was used without further purification. ESI-MS: 311.75 [M+H]+.

### c. 6-bromo-5-chloroimidazo[1,2-a]pyrazin-8-amine

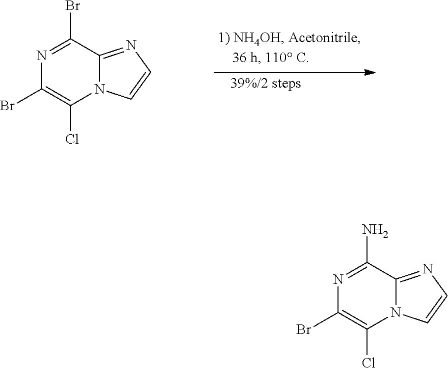 [SMILES: Clc1c(Br)nc(Br)c2nccn12 ; Nc1nc(Br)c(Cl)n2ccnc12]

A solution of 6,8-dibromo-5-chloroimidazo[1,2-a]pyrazine (815 mg, 2.62 mmol) in ammonium hydroxide (28-30%, 15 mL) was heated at 100° C. for 2 h in a pressure reactor. After that time, the reaction mixture was cooled down to r.t. and concentrated under reduced pressure. Crude material was diluted in methanol, filtered and rinsed with MeOH, EtOAc and DCM. The filtrate was adsorbed on silica gel and purified by flash chromatography on silica eluting with Hexane/EtOAc (1:0-0:1) to give the title compound (0.254 g, 1.03 mmol, 39% over 2 steps) as a beige solid. ESI-MS: 248.85 [M+H]+.

### d. 4-{8-amino-5-chloroimidazo[1,2-a]pyrazin-6-yl}benzonitrile

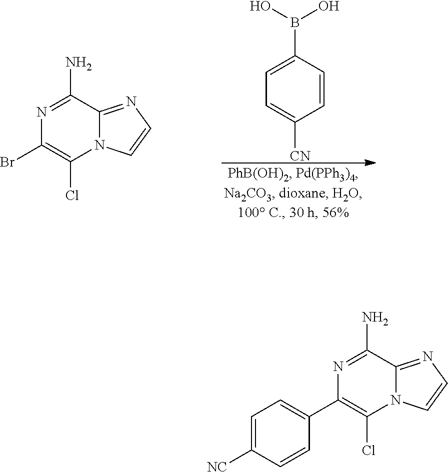 [SMILES: Nc1nc(Br)c(Cl)n2ccnc12 ; N#Cc1ccc(B(O)O)cc1 ; N#Cc1ccc(-c2nc(N)c3nccn3c2Cl)cc1]

In a pressure tube were mixed 6-bromo-5-chloroimidazo[1,2-a]pyrazin-8-amine (200 mg, 0.96 mmol), 4-cyanophenylboronic acid (155 mg, 1.06 mmol, 1.1 equiv.) and Na2CO3 (122 mg, 1.15 mmol, 1.2 equiv.) in a 10:1 mixture of 1,4-dioxane:water (6 mL). The reaction mixture was sparged with argon for 10 min. Pd(PPh3)4 (55 mg, 0.05 mmol, 5 mol %) was then added and the tube was capped and the reaction mixture was warmed to 100° C. and stirred overnight. After that time, remaining amount of SM was observed by LCMS therefore additional portion of boronic acid (15 mg, 0.11 mmol, 0.1 equiv.) and the reaction mixture was sparged for 5 min followed by the addition of another portion of Pd(PPh3)4 (27 mg, 0.025 mmol, 2.5 mol %). The tube was capped and the reaction mixture was warmed to 100° C. and stirred overnight. After that time, the reaction mixture was then cooled down to r.t., filtered through Celite® and rinsed with EtOAc. The organic solution was washed with water and brine and aqueous layer was extracted with EtOAc. Separated organic layer was dried over anhydrous sodium sulfate, filtered and concentrated under reduced pressure to afford the crude material as a brown residue. The obtained crude material was purified by flash chromatography on CN-silica eluting with hexane and DCM to lead to the title product (145 mg, 0.54 mmol, 56%) as a pale yellow solid. ESI-MS: 269.90 [M+H]+.

### e. 4-[8-amino-5-(3-chloro-4-hydroxyphenyl)imidazo[1,2-a]pyrazin-6-yl]benzonitrile

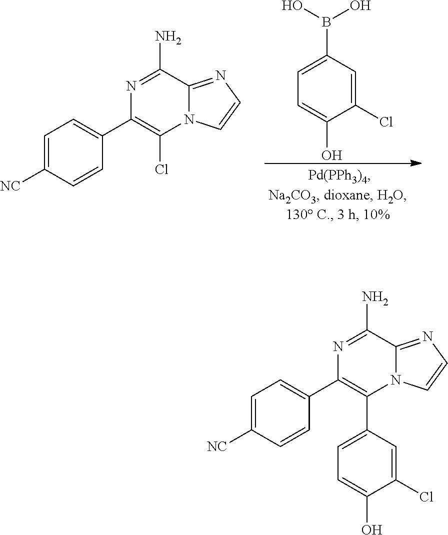 [SMILES: N#Cc1ccc(-c2nc(N)c3nccn3c2Cl)cc1 ; OB(O)c1ccc(O)c(Cl)c1 ; N#Cc1ccc(-c2nc(N)c3nccn3c2-c2ccc(O)c(Cl)c2)cc1]

In a pressure tube were mixed 4-{8-amino-5-chloroimidazo[1,2-a]pyrazin-6-yl}benzonitrile (145 mg, 0.54 mmol), 3-chloro-4-hydroxyphenylboronic acid (139 mg, 0.81 mmol, 1.5 equiv.) and Na2CO3 (114 mg, 1.08 mmol, 2 equiv.) in a 10:1 mixture of 1,4-dioxane:water (6 mL). The reaction mixture was sparged with argon for 10 min. Pd(PPh3)4 (31 mg, 0.03 mmol, 5 mol %) was then added and the tube was capped and the reaction mixture was warmed to 130° C. and stirred for 3 h. After that time, the reaction mixture was then cooled down to r.t., diluted with water and separated aqueous layer was extracted with DCM (3×10 mL), EtOAc (2×10 mL) then with 3:1 CHCl3:i-PrOH mixture (1×10 mL). Combined organic layers were dried over anhydrous sodium sulfate, filtered and concentrated under reduced pressure to afford the crude material as a brown residue. The obtained crude material was purified by flash chromatography on silica eluting with DCM:MeOH (10:0-9:1). Additional purification by HPLC led to the title product (20 mg, 0.055 mmol, 10%) as a white solid. ESI-MS: 362.5 [M+H]+. 1H NMR (300 MHz, DMSO-d6) δ 7.72 (d, J=8.0 Hz, 2H), 7.54 (s, 1H), 7.48 (d, J=8.0 Hz, 2H), 7.41 (s, 1H), 7.36 (s, 1H), 7.13 (d, J=9.4 Hz, 3H), 7.04 (d, J=8.3 Hz, 1H).

### Example 21: 4-{8-amino-6-phenylimidazo[1,2-a]pyrazin-5-yl}-N-methylpyridin-2-amine

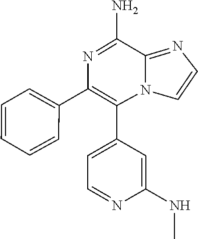 [SMILES: CNc1cc(-c2c(-c3ccccc3)nc(N)c3nccn23)ccn1]

The title compound was synthesized following the approach outlined in Procedure D, Conditions A substituting N-methyl-4-(tetramethyl-1,3,2-dioxaborolan-2-yl)pyridin-2-amine for 5-(tetramethyl-1,3,2-dioxaborolan-2-yl)-1H-indazole in step (d) and using the conditions B described in Procedure B to lead to the title compound (42 mg, 0.28 mmol, 68%) as an off-white solid. ESI-MS: 317.2 [M+H]+. 1H NMR (400 MHz, DMSO-d6) δ 8.03 (d, J=5.1 Hz, 1H), 7.53 (s, 1H), 7.47 (s, 1H), 7.42-7.31 (m, 2H), 7.31-7.18 (m, 3H), 7.18-7.03 (m, 2H), 6.62-6.53 (m, 1H), 6.47 (d, J=4.7 Hz, 1H), 6.36 (s, 1H), 2.71 (d, J=4.6 Hz, 3H).

### Example 22: 4-{8-amino-2,6-diphenylimidazo[1,2-a]pyrazin-5-yl}-2-chlorophenol

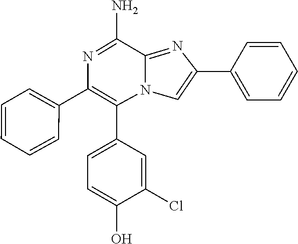 [SMILES: Nc1nc(-c2ccccc2)c(-c2ccc(O)c(Cl)c2)n2cc(-c3ccccc3)nc12]

The title compound was synthesized following the approach outlined in Procedure B, Conditions B in step (c) and substituting 2-bromoacetophenone for 2-chloroacetaldehyde in step (e) and performing this step at 125° C. for 3 days and performing the reaction in step (f) at 110° C. for 2 weeks to lead to the title product (12 mg, 0.03 mmol, 11%) as a white solid. ESI-MS: 413.6 [M+H]+. 1H NMR (400 MHz, DMSO-d6) δ 8.48 (s, 1H), 8.04-7.97 (m, 2H), 7.85 (s, 1H), 7.44-7.37 (m, 2H), 7.35-7.29 (m, 4H), 7.27-7.12 (m, 4H), 7.04 (d, J=8.2 Hz, 3H).

### Example 23: 4-[8-amino-6-(3-fluorophenyl)imidazo[1,2-a]pyrazin-5-yl]phenol

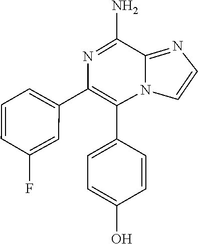 [SMILES: Nc1nc(-c2cccc(F)c2)c(-c2ccc(O)cc2)n2ccnc12]

The title compound was synthesized following the approach outlined in Procedure D, Conditions A substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 4-hydroxyphenylboronic acid for 5-(tetramethyl-1,3,2-dioxaborolan-2-yl)-1H-indazole in step (d) using the conditions B described in Procedure B to afford the title compound (37 mg, 0.12 mmol, 47%) as an off-white solid. ESI-MS: 321 [M+H]+. 1H NMR (300 MHz, DMSO-d6) δ 9.79 (s, 1H), 7.51 (d, J=1.1 Hz, 1H), 7.33 (d, J=1.1 Hz, 1H), 7.26-7.17 (m, 1H), 7.17 (d, J=8.5 Hz, 2H), 7.12-6.96 (m, 5H), 6.83 (d, J=8.5 Hz, 2H).

### Example 24; 4-[8-amino-6-(3-fluorophenyl)imidazo[1,2-a]pyrazin-5-yl]-N-methylpyridin-2-amine

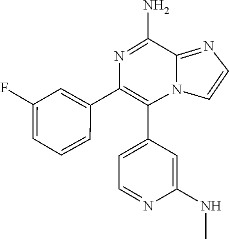 [SMILES: CNc1cc(-c2c(-c3cccc(F)c3)nc(N)c3nccn23)ccn1]

The title compound was synthesized following the approach outlined in Procedure D, Conditions A substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting N-methyl-4-(tetramethyl-1,3,2-dioxaborolan-2-yl)pyridin-2-amine for 5-(tetramethyl-1,3,2-dioxaborolan-2-yl)-1H-indazole in step (d) and using the conditions B described in Procedure B to lead to the title compound (80 mg, 0.22 mmol, 57%) as hydrochloride salt as yellow solid. ESI-MS: 335.2 [M+H]+. 1H NMR (400 MHz, DMSO-d6) δ 9.32 (br s, 1H), 8.84 (br s, 2H), 8.08-7.87 (m, 3H), 7.49-7.36 (m, 1H), 7.33-7.24 (m, 2H), 7.24-7.08 (m, 2H), 6.73 (dd, J=6.6, 1.4 Hz, 1H), 2.97 (s, 3H).

### Example 25:8-amino-5-(3-chloro-4-hydroxyphenyl)-6-phenylimidazo[1,2-a]pyrazine-2-carboxamide

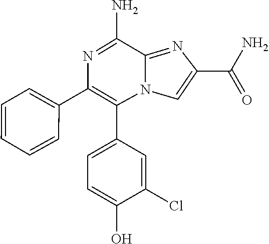 [SMILES: NC(=O)c1cn2c(-c3ccc(O)c(Cl)c3)c(-c3ccccc3)nc(N)c2n1]

The title compound was synthesized following the approach outlined in Procedure B, Conditions B in step (c) and substituting 3-bromopyruvic acid ethyl ester for 2-chloroacetaldehyde in step (e) and performing this step at 60° C. overnight and performing the reaction in step (f) without acetonitrile at 100° C. for 5 h to lead to the title compound (2.6 mg, 6.8 μmol, 13%) as pale yellow solid. ESI-MS: 380.6 [M+H]+. 1H NMR (300 MHz, DMSO-d6) δ 8.49 (s, 1H), 7.74 (s, 1H), 7.68-7.54 (m, 1H), 7.54-7.41 (m, 1H), 7.40-7.29 (m, 3H), 7.28-7.04 (m, 6H), 6.99 (d, J=8.3 Hz, 1H).

### Example 26: 4-[8-amino-6-(4-fluorophenyl)imidazo[1,2-a]pyrazin-5-yl]-N-methylpyridin-2-amine

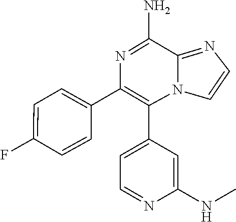 [SMILES: CNc1cc(-c2c(-c3ccc(F)cc3)nc(N)c3nccn23)ccn1]

The title compound was synthesized following the approach outlined in Procedure D, Conditions A substituting 6-chloro-5-(4-fluorophenyl)pyrazin-2-amine [prepared following the approach outlined in Procedure B substituting 4-fluorophenylboronic acid for phenylboronic acid in step (b)] for 6-chloro-5-phenylpyrazin-2-amine in step (a) and performing the reaction described in step (c) for 16 h and substituting 2-(N-methylamine)pyridine-4-boronic acid for 5-(tetramethyl-1,3,2-dioxaborolan-2-yl)-1H-indazole in step (d) and performing this step for 4 h to lead to the title compound (13 mg, 0.04 mmol, 9%) as hydrochloride salt as pale yellow solid. ESI-MS: 335.3 [M+H]+. 1H NMR (400 MHz, DMSO-d6) δ 8.98 (s, 1H), 8.22 (s, 1H), 7.99-7.84 (m, 2H), 7.79 (s, 1H), 7.49-7.37 (m, 2H), 7.28-7.15 (m, 2H), 7.08 (s, 1H), 6.67 (dd, J=6.6, 1.6 Hz, 1H), 2.93 (s, 3H).

### Example 27: 6-(3-fluorophenyl)-5-(quinolin-6-yl)imidazo[1,2-a]pyrazin-8-amine

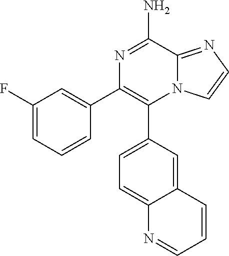 [SMILES: Nc1nc(-c2cccc(F)c2)c(-c2ccc3ncccc3c2)n2ccnc12]

The title compound was synthesized following the approach outlined in Procedure D, Conditions A substituting 6-chloro-5-(4-fluorophenyl)pyrazin-2-amine [prepared following the approach outlined in Procedure B substituting 3-fluorophenylboronic acid for phenylboronic acid in step (b)] for 6-chloro-5-phenylpyrazin-2-amine in step (a) and performing the reaction described in step (c) for 16 h and substituting 6-(tetramethyl-1,3,2-dioxaborolan-2-yl)quinoline for 5-(tetramethyl-1,3,2-dioxaborolan-2-yl)-1H-indazole in step (d) and performing this step for 4 h to lead to the title compound (62 mg, 0.16 mmol, 41%) as hydrochloride salt as an orange solid. ESI-MS: 356.3 [M+H]+. 1H NMR (400 MHz, DMSO-d6) δ 9.15 (dd, J=4.7, 1.6 Hz, 1H), 9.15-8.75 (br s, 3H), 8.73 (d, J=8.5 Hz, 1H), 8.33-8.22 (m, 2H), 7.94 (d, J=1.4 Hz, 1H), 7.89 (dd, J=8.7, 1.9 Hz, 1H), 7.83 (dd, J=8.4, 4.6 Hz, 1H), 7.75 (d, J=1.4 Hz, 1H), 7.34-7.20 (m, 2H), 7.19-7.10 (m, 2H).

### Example 28: 5-(3,5-dichlorophenyl)-6-(4-fluorophenyl)imidazo[1,2-a]pyrazin-8-amine

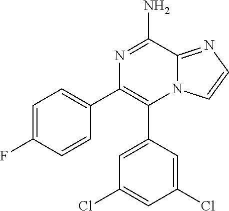 [SMILES: Nc1nc(-c2ccc(F)cc2)c(-c2cc(Cl)cc(Cl)c2)n2ccnc12]

The title compound was synthesized following the approach outlined in Procedure B, Conditions B substituting 4-fluorophenylboronic acid for phenylboronic acid in step (b) and heating the reaction for 6.5 h and substituting 3,5-dichlorophenylboronic acid for (3-chloro-4-hydroxyphenyl)-boronic acid in step (c) and substituting 2-bromo-1,1-dimethoxyethane for 2-chloroacetaldehyde in step (e) and performing this step in 10:1 ethanol:water to afford the title compound as a hydrochloride salt (pale yellow solid, 7 mg, 14%). ESI-MS: 373.70 [M+H]⁺. ¹H NMR (300 MHz, CDCl₃) δ 7.81 (s, 1H), 7.55 (s, 1H), 7.49 (s, 1H), 7.41 (s, 2H), 7.28 (s, 4H), 7.09 (m, 2H).

### Example 29: 6-(2-fluorophenyl)-5-[2-methyl-6-(trifluoromethyl)pyridin-4-yl]imidazo[1,2-a]pyrazin-8-amine

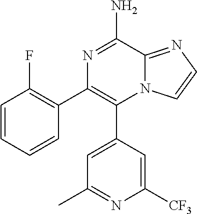 [SMILES: Cc1cc(-c2c(-c3ccccc3F)nc(N)c3nccn23)cc(C(F)(F)F)n1]

The title compound was synthesized following the approach outlined in Procedure B, Conditions A substituting 2-fluorophenylboronic acid for phenylboronic acid in step (b) and heating the reaction overnight and substituting 2-methyl-4-(tetramethyl-1,3,2-dioxaborolan-2-yl)-6-(trifluoromethyl)pyridine (Procedure A1) for (3-chloro-4-hydroxyphenyl)-boronic acid in step (c) and performing the reaction at 150° C. for 3 h and substituting 2-bromo-1,1-dimethoxyethane for 2-chloroacetaldehyde in step (e) and performing this step in 10:1 ethanol:water and heating the reaction in step (f) at 110° C. under microwave irradiation for 1 h to afford the title compound as a hydrochloride salt (white solid, 46 mg, 23%). ESI-MS: 388.30 [M+H]⁺. ¹H NMR (400 MHz, DMSO-d6) δ 9.08 (s, 2H), 8.03 (d, J=3.1 Hz, 2H), 7.67 (s, 1H), 7.56 (s, 1H), 7.52-7.42 m, 2H), 7.28-7.16 (m, 2H), 2.54 (s, 3H).

### Example 30:3-[8-amino-5-(3,5-dichlorophenyl)imidazo[1,2-a]pyrazin-6-yl]benzonitrile

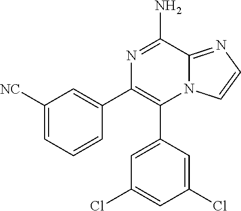 [SMILES: N#Cc1cccc(-c2nc(N)c3nccn3c2-c2cc(Cl)cc(Cl)c2)c1]

The title compound was synthesized following the approach outlined in Procedure B, Conditions B substituting 3-cyanophenylboronic acid for phenylboronic acid in step (b) and performing the reaction for 6.5 h and substituting 3,5-dichlorophenylboronic acid for (3-chloro-4-hydroxyphenyl)-boronic acid in step (c) and performing the reaction in step (e) without additional solvent than water contained in reagent and performing the reaction in step (f) at 90° C. overnight without additional solvent than water contained in reagent to afford the title compound as a hydrochloride salt (beige solid, 18 mg, 21%). ESI-MS: 380.70 [M+H]⁺. ¹H NMR (300 MHz, CD₃OD) δ 7.86 (m, 1H), 7.84 (d, J=1.3 Hz, 1H), 7.80 (dd, J=7.7, 1.4 Hz, 1H), 7.69 (d, J=1.3 Hz, 1H), 7.66 (dd, J=7.7, 1.6 Hz, 1H), 7.64 (t, J=1.9 Hz, 1H), 7.58 (d, J=7.8 Hz, 1H), 7.49 (d, J=1.9 Hz, 2H).

### Example 31: 5-(3,5-dichlorophenyl)-6-phenylimidazo[1,2-a]pyrazin-8-amine

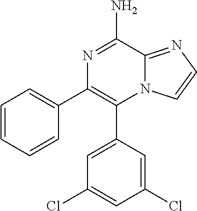 [SMILES: Nc1nc(-c2ccccc2)c(-c2cc(Cl)cc(Cl)c2)n2ccnc12]

The title compound was synthesized following the approach outlined in Procedure B, Conditions B substituting 3,5-dichlorophenylboronic acid for (3-chloro-4-hydroxyphenyl)-boronic acid in step (c) and performing this step at 150° C. for 4 h and performing the reaction in step (f) at 100° C. under microwave irradiation for 1 h to afford the title compound as a hydrochloride salt (white solid, 55 mg, 24%). ESI-MS: 355.70 [M+H]⁺. ¹H NMR (300 MHz, DMSO-d6) δ 7.67 (t, J=1.9 Hz, 1H), 7.54 (d, J=1.2 Hz, 1H), 7.48 (m, 3H), 7.31-7.23 (m, 5H), 7.20 (s, 2H).

### Example 32:4-{8-amino-6-[3-(trifluoromethyl)phenyl]imidazo[1,2-a]pyrazin-5-yl}-2-chlorophenol

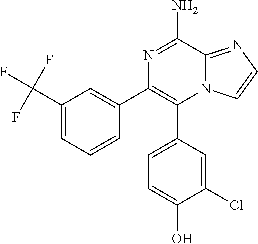 [SMILES: Nc1nc(-c2cccc(C(F)(F)F)c2)c(-c2ccc(O)c(Cl)c2)n2ccnc12]

The title compound was synthesized following the approach outlined in Procedure B, Conditions A in step (c) and substituting 3-trifluoromethylphenylboronic acid for phenyl boronic acid in step (b) and performing the reaction in step (c) at 130° C. for 3 h and performing the reaction in step (e) at 100° C. for 45 min under microwave irradiation and performing the reaction in step (f) at 100° C. overnight to afford the title compound as a hydrochloride salt (white solid, 10 mg, 6%). ESI-MS: 405.50 [M+H]⁺. ¹H NMR (400 MHz, DMSO-d6) δ 7.67 (s, 1H), 7.59-7.51 (m, 3H), 7.45 (t, J=7.8 Hz, 1H), 7.40 (d, J=2.1 Hz, 2H), 7.17 (dd, J=7.4, 2.9 Hz, 3H), 7.03 (d, J=8.3 Hz, 1H).

### Example 33:5-(3-chloro-1H-indazol-5-yl)-6-phenylimidazo[1,2-a]pyrazin-8-amine

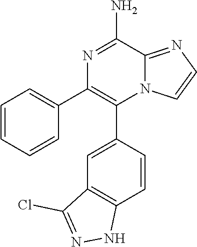 [SMILES: Nc1nc(-c2ccccc2)c(-c2ccc3[nH]nc(Cl)c3c2)n2ccnc12]

The title compound was synthesized following the approach outlined in Procedure D substituting 3-chloro-5-(tetramethyl-1,3,2-dioxaborolan-2-yl)-1H-indazole for 5-(tetramethyl-1,3,2-dioxaborolan-2-yl)-1H-indazole in step (d) and substituting Pd(PPh₃)₄ for Pd(dppf)Cl₂.DCM and substituting potassium phosphate for sodium carbonate in step (d) and performing this step at 90° C. overnight to afford the title compound as a yellow solid (12 mg, 0.033 mmol, 9%). ESI-MS: 361.40 [M+H]⁺. ¹H NMR (400 MHz, DMSO-d6) δ13.46 (s, 1H), 7.66 (d, J=1.4 Hz, 1H), 7.62 (d, J=8.6 Hz, 1H), 7.51 (d, J=1.2 Hz, 1H), 7.41 (dd, J=8.7, 1.6 Hz, 1H), 7.33 (d, J=1.2 Hz, 1H), 7.30-7.26 (m, 2H), 7.18-7.12 (m, 3H), 7.07 (s, 2H).

### Example 34: 5-(2-chloro-6-methylpyridin-4-yl)-6-(3-fluorophenyl)imidazo[1,2-a]pyrazin-8-amine

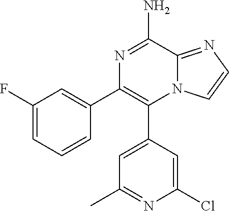 [SMILES: Cc1cc(-c2c(-c3cccc(F)c3)nc(N)c3nccn23)cc(Cl)n1]

The title compound was synthesized following the approach outlined in Procedure B, Conditions B in step (c) and substituting 3-fluorophenylboronic acid for phenyl boronic acid in step (b) and performing the reaction in step (c) for 3 h and performing the reaction in step (d) at −5° C. for 2 h slowly raising temperature to ambient and stirring overnight and performing the reaction in step (e) at 100° C. for 45 min under microwave irradiation and performing the reaction in step (f) at 100° C. overnight to afford, after HPLC purification using formic acid, the title compound as formate salt (white solid, 22 mg, 18%). ESI-MS: 354.50 [M+H]⁺. ¹H NMR (300 MHz, DMSO-d6) δ 7.58 (d, J=1.2 Hz, 1H), 7.56 (d, J=1.2 Hz, 1H), 7.37 (d, J=1.2 Hz, 1H), 7.33-7.32 (s, 2H), 7.30 (s, 1H), 7.28-7.23 (m, 1H), 7.19-7.06 (m, 2H), 7.03 (dt, J=7.7, 1.3 Hz, 1H), 2.44 (s, 3H).

### Example 35:5-(2-chloro-6-methylpyridin-4-yl)-6-(4-fluorophenyl)imidazo[1,2-a]pyrazin-8-amine

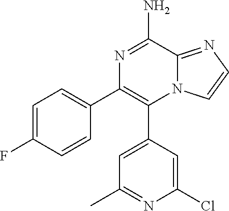 [SMILES: Cc1cc(-c2c(-c3ccc(F)cc3)nc(N)c3nccn23)cc(Cl)n1]

The title compound was synthesized following the approach outlined in Procedure B, Conditions B in step (c) substituting 4-fluorophenylboronic acid for phenylboronic acid in step (b) and substituting 2-chloro-6-methyl-4-(tetramethyl-1,3,2-dioxaborolan-2-yl)pyridine for (3-chloro-4-hydroxyphenyl)boronic acid in step (c) and performing this step at 130° C. for 1 h and substituting 2-bromo-1,1-dimethoxyethane for 2-chloroacetaldehyde in step (e) and performing this step at 120° C. for 45 min and performing the reaction in step (f) in 1,4-dioxane at 80° C. overnight to lead to the title compound (11.5 mg, 0.03 mmol, 8%) as a white solid. ESI-MS: 354.5 [M+H]⁺. ¹H NMR (400 MHz, CD₃OD) δ 7.70 (s, 1H), 7.66 (d, J=1.2 Hz, 1H), 7.40 (dd, J=8.8, 5.3 Hz, 2H), 7.31 (d, J=1.6 Hz, 1H), 7.28 (d, J=1.2 Hz, 1H), 7.14-7.07 (m, 2H), 2.50 (s, 3H).

### Example 36: 4-[8-amino-6-(3-fluorophenyl)imidazo[1,2-a]pyrazin-5-yl]benzamide

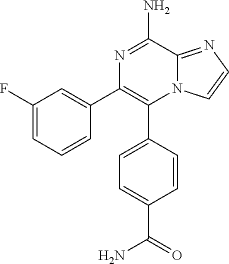 [SMILES: NC(=O)c1ccc(-c2c(-c3cccc(F)c3)nc(N)c3nccn23)cc1]

The title compound was synthesized following the approach outlined in Procedure D substituting 6-chloro-5-(4-fluorophenyl)pyrazin-2-amine [prepared following the approach outlined in Procedure B substituting 4-fluorophenylboronic acid for phenylboronic acid in step (b)] for 6-chloro-5-phenylpyrazin-2-amine in step (a) and performing the reaction described in step (c) for 16 h and substituting (4-carbamoylphenyl)boronic acid for 5-(tetramethyl-1,3,2-dioxaborolan-2-yl)-1H-indazole in step (d) and performing this step for 4 h to lead to the title compound (65 mg, 0.19 mmol, 62%) as an off-white solid. ESI-MS: 348.2 [M+H]⁺. ¹H NMR (400 MHz, DMSO-d6) δ 8.07 (s, 1H), 7.93 (d, J=8.3 Hz, 2H), 7.54 (d, J=1.1 Hz, 1H), 7.52-7.43 (m, 3H), 7.37 (d, J=1.2 Hz, 1H), 7.26-7.15 (m, 3H), 7.12-6.98 (m, 3H).

### Example 37: 5-(3-methyl-1H-indazol-5-yl)-6-phenylimidazo[1,2-a]pyrazin-8-amine

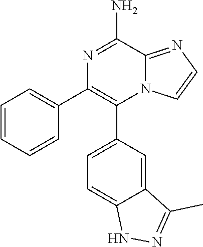 [SMILES: Cc1n[nH]c2ccc(-c3c(-c4ccccc4)nc(N)c4nccn34)cc12]

The title compound was synthesized following the approach outlined in Procedure E substituting phenylboronic acid for 4-cyanophenylboronic acid in step (d) and substituting 3-methyl-5-(tetramethyl-1,3,2-dioxaborolan-2-yl)-1H-indazole for (3-chloro-4-hydroxyphenyl)boronic acid in step (e) and using the conditions described in Procedure D step (d) to lead to the title compound (30 mg, 0.09 mmol, 27%) as a white solid. ESI-MS: 341.3 [M+H]⁺. ¹H NMR (400 MHz, DMSO-d6) δ 12.79 (s, 1H), 7.82-7.72 (m, 1H), 7.52-7.46 (m, 2H), 7.33-7.28 (m, 3H), 7.24 (dd, J=8.5, 1.6 Hz, 1H), 7.18-7.11 (m, 3H), 7.02 (s, 2H), 2.42 (s, 3H).

### Example 38: 5-(1H-indol-5-yl)-6-phenylimidazo[1,2-a]pyrazin-8-amine

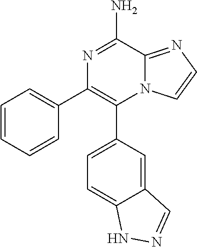 [SMILES: Nc1nc(-c2ccccc2)c(-c2ccc3[nH]ncc3c2)n2ccnc12]

The title compound was synthesized following the approach outlined in Procedure E substituting phenylboronic acid for 4-cyanophenylboronic acid in step (d) and substituting 5-(tetramethyl-1,3,2-dioxaborolan-2-yl)-1H-indole for (3-chloro-4-hydroxyphenyl)boronic acid in step (e) and using the conditions described in Procedure D step (d) to lead to the title compound (40 mg, 0.12 mmol, 38%) as a white solid. ESI-MS: 326.2 [M+H]⁺. ¹H NMR (400 MHz, DMSO-d6) δ 11.27 (s, 1H), 7.53-7.51 (m, 1H), 7.48 (d, J=1.1 Hz, 1H), 7.46 (dt, J=8.3, 0.9 Hz, 1H), 7.39 (t, J=2.8 Hz, 1H), 7.34-7.28 (m, 2H), 7.25 (d, J=1.2 Hz, 1H), 7.15-7.08 (m, 3H), 7.05 (dd, J=8.3, 1.7 Hz, 1H), 6.95 (s, 2H), 6.44-6.39 (m, 1H).

### Example 39: 6-(3-fluorophenyl)-5-(pyridin-4-yl)imidazo[1,2-a]pyrazin-8-amine

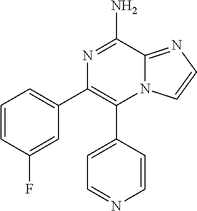 [SMILES: Nc1nc(-c2cccc(F)c2)c(-c2ccncc2)n2ccnc12]

The title compound was synthesized following the approach outlined in Procedure D substituting 6-chloro-5-(4-fluorophenyl)pyrazin-2-amine [prepared following the approach outlined in Procedure B substituting 4-fluorophenylboronic acid for phenylboronic acid in step (b)] for 6-chloro-5-phenylpyrazin-2-amine in step (a) and performing the reaction described in step (c) for 16 h and substituting 4-pyridinylboronic acid for 5-(tetramethyl-1,3,2-dioxaborolan-2-yl)-1H-indazole in step (d) to lead to the title compound (39 mg, 0.11 mmol, 45%) as formate salt as a pale yellow solid. ESI-MS: 306.2 [M+H]⁺. ¹H NMR (400 MHz, DMSO-d6) δ 8.67-8.60 (m, 2H), 8.46 (s, 1H), 7.56 (d, J=1.1 Hz, 1H), 7.49 (d, J=1.2 Hz, 1H), 7.45-7.38 (m, 2H), 7.29 (s, 2H), 7.14 (dt, J=7.9, 6.1 Hz, 1H), 7.15-6.97 (m, 3H).

### Example 40: 6-(1-methyl-1H-pyrazol-3-yl)-5-[2-methyl-6-(trifluoromethyl)pyridin-4-yl]imidazo[1,2-a]pyrazin-8-amine

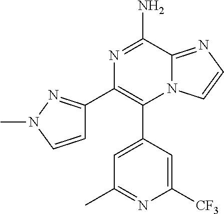 [SMILES: Cc1cc(-c2c(-c3ccn(C)n3)nc(N)c3nccn23)cc(C(F)(F)F)n1]

The title compound was synthesized following the approach outlined in Procedure E substituting 1-methyl-3-(4,4,5,5-tetramethyl-1,3,2-dioxaborolan-2-yl)-1H-pyrazole for 4-cyanophenylboronic acid in step (d) and performing the reaction described in step (d) for 20 h and substituting 2-methyl-4-(tetramethyl-1,3,2-dioxaborolan-2-yl)-6-(trifluoromethyl)pyridine (Procedure A1) for (3-chloro-4-hydroxyphenyl)boronic acid in step (e) to lead to the title compound (74 mg, 0.30 mmol, 44%) as a hydrochloride salt as a beige solid. ESI-MS: 374.4 [M+H]⁺. ¹H NMR (400 MHz, DMSO-d6) δ 7.89-7.84 (m, 2H), 7.81 (s, 1H), 7.76 (d, J=1.3 Hz, 1H), 7.72 (d, J=2.4 Hz, 1H), 5.70 (s, 1H), 3.85 (s, 3H), 2.67 (s, 3H).

### Example 41: 5-(3-fluoro-1H-indazol-5-yl)-6-phenylimidazo[1,2-a]pyrazin-8-amine

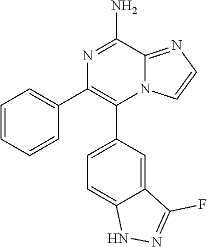 [SMILES: Nc1nc(-c2ccccc2)c(-c2ccc3[nH]nc(F)c3c2)n2ccnc12]

The title compound was synthesized following the approach outlined in Procedure D, Conditions A substituting 3-fluoro-5-(tetramethyl-1,3,2-dioxaborolan-2-yl)-1H-indazole for 5-(tetramethyl-1,3,2-dioxaborolan-2-yl)-1H-indazole in step (d) to lead to the title compound (13 mg, 0.04 mmol, 12%) as a white solid. ESI-MS: 345.2 [M+H]⁺. ¹H NMR (400 MHz, DMSO-d6) δ 12.72 (s, 1H), 7.74 (s, 1H), 7.55-7.49 (m, 2H), 7.40-7.34 (m, 2H), 7.31-7.26 (m, 2H), 7.19-7.12 (m, 3H), 7.07 (s, 2H).

### Example 42: 4-[8-amino-6-(1-methyl-1H-pyrazol-3-yl)imidazo[1,2-a]pyrazin-5-yl]-2-chlorophenol

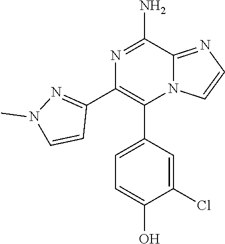 [SMILES: Cn1ccc(-c2nc(N)c3nccn3c2-c2ccc(O)c(Cl)c2)n1]

The title compound was synthesized following the approach outlined in Procedure E substituting 1-methyl-3-(4,4,5,5-tetramethyl-1,3,2-dioxaborolan-2-yl)-1H-pyrazole for 4-cyanophenylboronic acid in step (d) and performing the reaction described in step (d) for 30 h to lead to the title compound (33 mg, 0.09 mmol, 16%) as a hydrochloride salt as a beige solid. ESI-MS: 341.0 [M+H]⁺. ¹H NMR (400 MHz, DMSO-d6) δ 11.11 (s, 1H), 10.03 (s, 1H), 8.78 (s, 1H), 7.79 (d, J=1.2 Hz, 1H), 7.69 (d, J=2.4 Hz, 1H), 7.57 (d, J=1.2 Hz, 1H), 7.53 (d, J=1.8 Hz, 1H), 7.32-7.26 (m, 2H), 5.25 (d, J=2.4 Hz, 1H), 3.93 (s, 3H).

### Example 43: 5-(1-benzofuran-5-yl)-6-(3-fluorophenyl)imidazo[1,2-a]pyrazin-8-amine

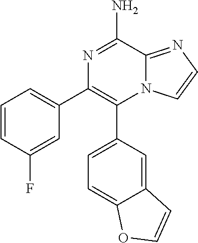 [SMILES: Nc1nc(-c2cccc(F)c2)c(-c2ccc3occc3c2)n2ccnc12]

The title compound was synthesized following the approach outlined in Procedure D, Conditions A substituting 6-chloro-5-(4-fluorophenyl)pyrazin-2-amine [prepared following the approach outlined in Procedure B substituting 4-fluorophenylboronic acid for phenylboronic acid in step (b)] for 6-chloro-5-phenylpyrazin-2-amine in step (a) and performing the reaction described in step (c) for 16 h and substituting 2-(1-benzofuran-5-yl)-4,4,5,5-tetramethyl-1,3,2-dioxaborolane for 5-(tetramethyl-1,3,2-dioxaborolan-2-yl)-1H-indazole in step (d) to lead to the title compound (47 mg, 0.14 mmol, 45%) as a white solid. ESI-MS: 345.2 [M+H]⁺. ¹H NMR (400 MHz, DMSO-d₆) δ 8.07 (d, J=2.2 Hz, 1H), 7.72-7.67 (m, 2H), 7.51 (d, J=1.2 Hz, 1H), 7.33 (dd, J=8.6, 1.7 Hz, 1H), 7.30 (d, J=1.2 Hz, 1H), 7.17 (td, J=8.0, 6.2 Hz, 1H), 7.13-7.07 (m, 3H), 7.05 (dt, J=7.9, 1.1 Hz, 1H), 7.01-6.93 (m, 2H).

### Example 44: 4-[8-amino-6-(2-fluorophenyl)imidazo[1,2-a]pyrazin-5-yl]-2-chlorophenol

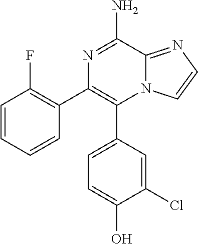 [SMILES: Nc1nc(-c2ccccc2F)c(-c2ccc(O)c(Cl)c2)n2ccnc12]

The title compound was synthesized following the approach outlined in Procedure B, Conditions B in step (c) performing this step (f) or 5 h and substituting 2-fluorophenylboronic acid for phenylboronic acid in step (b) and performing this step (a) at 100° C. for 45 min to lead to the title compound (90 mg, 0.25 mmol, 26%) as a light yellow solid. ESI-MS: 355.4 [M+H]⁺. ¹H NMR (400 MHz, DMSO-d6) δ 10.89 (s, 1H), 9.12 (s, 2H), 7.95 (s, 1H), 7.76 (d, J=1.3 Hz, 1H), 7.51-7.39 (m, 2H), 7.36 (d, J=2.1 Hz, 1H), 7.27-7.20 (m, 2H), 7.15 (dd, J=8.4, 2.1 Hz, 1H), 7.05 (d, J=8.4 Hz, 1H).

### Example 45: 6-(3-fluorophenyl)-5-(2-methoxypyridin-4-yl)imidazo[1,2-a]pyrazin-8-amine

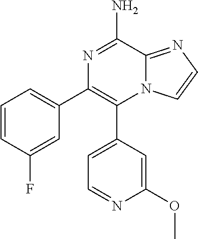 [SMILES: COc1cc(-c2c(-c3cccc(F)c3)nc(N)c3nccn23)ccn1]

The title compound was synthesized following the approach outlined in Procedure D, Conditions A substituting 6-chloro-5-(4-fluorophenyl)pyrazin-2-amine [prepared following the approach outlined in Procedure B substituting 4-fluorophenylboronic acid for phenylboronic acid in step (b)] for 6-chloro-5-phenylpyrazin-2-amine in step (a) and performing the reaction described in step (c) for 16 h and substituting (2-methoxypyridin-4-yl)boronic acid for 5-(tetramethyl-1,3,2-dioxaborolan-2-yl)-1H-indazole in step (d) and performing this step (f) or 4 h to lead to the title compound (25 mg, 0.07 mmol, 25%) as a beige solid. ESI-MS: 336.2 [M+H]⁺. ¹H NMR (400 MHz, DMSO-d6) δ 8.69 (s, 2H), 8.27 (d, J=5.2 Hz, 1H), 7.88 (s, 1H), 7.74 (d, J=1.0 Hz, 1H), 7.43-7.33 (m, 1H), 7.26-7.17 (m, 2H), 7.15 (d, J=7.6 Hz, 1H), 7.01 (dd, J=5.2, 1.2 Hz, 1H), 6.91 (s, 1H), 3.86 (s, 3H).

### Example 46: 5-(2-fluoro-6-methylpyridin-4-yl)-6-(3-fluorophenyl)imidazo[1,2-a]pyrazin-8-amine

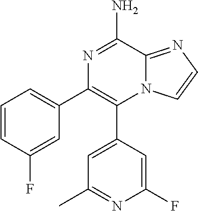 [SMILES: Cc1cc(-c2c(-c3cccc(F)c3)nc(N)c3nccn23)cc(F)n1]

The title compound was synthesized following the approach outlined in Procedure D, Conditions A substituting 6-chloro-5-(4-fluorophenyl)pyrazin-2-amine [prepared following the approach outlined in Procedure B substituting 4-fluorophenylboronic acid for phenylboronic acid in step (b)] for 6-chloro-5-phenylpyrazin-2-amine in step (a) and performing the reaction described in step (c) for 16 h and substituting (2-fluoro-6-methylpyridin-4-yl)boronic acid for 5-(tetramethyl-1,3,2-dioxaborolan-2-yl)-1H-indazole in step (d) and performing this step (f) or 4 h to lead to the title compound (10 mg, 0.03 mmol, 10%) as a beige solid. ESI-MS: 338.2 [M+H]⁺. ¹H NMR (400 MHz, DMSO-d₆) δ 8.14 (s, 2H), 7.80-7.72 (m, 2H), 7.40-7.30 (m, 1H), 7.27 (s, 1H), 7.24-7.15 (m, 2H), 7.10 (d, J=7.6 Hz, 1H), 7.04 (s, 1H), 2.41 (s, 3H).

### Example 47: 4-{8-amino-2-methyl-6-phenylimidazo[1,2-a]pyrazin-5-yl}-2-chlorophenol

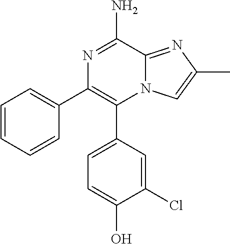 [SMILES: Cc1cn2c(-c3ccc(O)c(Cl)c3)c(-c3ccccc3)nc(N)c2n1]

The title compound was synthesized following the approach outlined in Procedure B, Conditions B in step (c) and substituting chloroacetone for 2-chloroacetaldehyde in step (e) and performing this step without solvent at 100° C. for 2 days to lead to the title compound (white solid, 25 mg, 0.07 mmol, 22%/2 steps). ESI-MS: 351.5 [M+H]⁺. ¹H NMR (300 MHz, DMSO-d6) δ 10.56 (s, 1H), 7.29 (m, 3H), 7.25-7.16 (m, 3H), 7.17-7.06 (m, 2H), 6.99 (d, J=8.3 Hz, 1H), 6.90 (s, 2H), 2.30 (s, 3H).

### Example 48: 4-{8-amino-6-phenylimidazo[1,2-a]pyrazin-5-yl}-6-fluoro-N-methylpyridin-2-amine

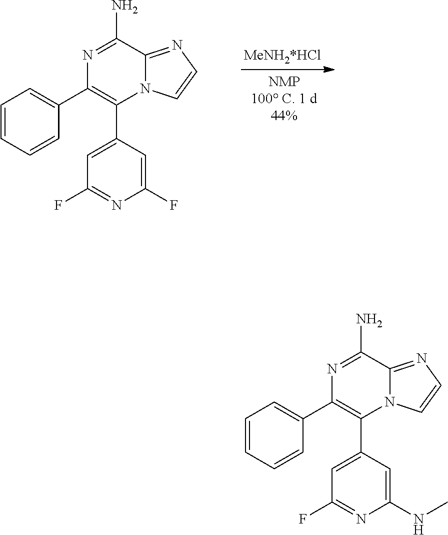 [SMILES: Nc1nc(-c2ccccc2)c(-c2cc(F)nc(F)c2)n2ccnc12 ; CNc1cc(-c2c(-c3ccccc3)nc(N)c3nccn23)cc(F)n1]

A) 5-(2,6-Difluoropyridin-4-yl)-6-phenylimidazo[1,2-a]pyrazin-8-amine was synthesized following the approach outlined in Procedure D, Conditions A substituting (2,6-difluoropyridin-4-yl)boronic acid for 5-(tetramethyl-1,3,2-dioxaborolan-2-yl)-1H-indazole in step (d) heating at 150° C. for 4 h to lead to the 5-(2,6-difluoropyridin-4-yl)-6-phenylimidazo[1,2-a]pyrazin-8-amine (285 mg, 72%) as a brown solid. ESI-MS: 324.1 [M+H]+.

B) In a dry pressure tube was placed 5-(2,6-difluoropyridin-4-yl)-6-phenylimidazo[1,2-a]pyrazin-8-amine (55 mg, 0.17 mmol) then it was dissolved in NMP (0.3 mL) and then methylamine hydrochloride (23 mg, 0.34 mmol) was added. Reaction was then heated at 100° C. for 1 d. After cooling to room temperature, the crude reaction mixture was concentrated under reduced pressure. The resulting residue was purified by column chromatography, eluting with hexane/ethyl acetate. The title product was obtained as a white solid (25 mg, 44%). ESI-MS: 335.2 [M+H]+. ¹H NMR (400 MHz, DMSO-d6) δ 7.57 (d, J=1.1 Hz, 1H), 7.54 (d, J=1.1 Hz, 1H), 7.40-7.35 (m, 2H), 7.31-7.23 (m, 3H), 7.18 (s, 2H), 7.05-6.97 (m, 1H), 6.27-6.22 (m, 1H), 6.17-6.12 (m, 1H), 2.68 (d, J=4.8 Hz, 3H).

### Example 49: 3-{8-amino-5-[2-(methylamino)pyridin-4-yl]imidazo[1,2-a]pyrazin-6-yl}benzonitrile

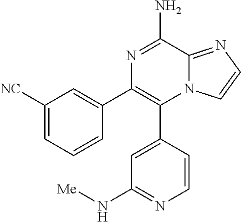 [SMILES: CNc1cc(-c2c(-c3cccc(C#N)c3)nc(N)c3nccn23)ccn1]

The title compound was synthesized following the approach outlined in Procedure D, substituting 6-chloro-5-(3-cyanophenyl)pyrazin-2-amine [Procedure B, step (b) substituting 3-cyanophenylboronic acid for phenylboronic acid] for 6-chloro-5-phenylpyrazin-2-amine in step (a) and substituting (2-methylaminopyridin-4-yl)boronic acid for 5-(tetramethyl-1,3,2-dioxaborolan-2-yl)-1H-indazole in step (d) and using the conditions A described in Procedure D heating at 150° C. for 3 h to lead to the title compound (beige solid, 76 mg, 57%). ESI-MS: 342.3 [M+H]+. 1H NMR (400 MHz, DMSO-d6) δ 8.06 (dd, J=5.2, 0.8 Hz, 1H), 7.83-7.78 (m, 1H), 7.75-7.68 (m, 1H), 7.62-7.57 (m, 1H), 7.56 (d, J=1.2 Hz, 1H), 7.50 (d, J=1.2 Hz, 1H), 7.49-7.42 (m, 1H), 7.25 (s, 2H), 6.62 (dd, J=4.7 Hz, 1H), 6.49 (dd, J=5.2, 1.5 Hz, 1H), 6.41-6.35 (m, 1H), 2.72 (d, J=4.8 Hz, 3H).

### Example 50:5-[2-methyl-6-(trifluoromethyl)pyridin-4-yl]-6-(naphthalen-2-yl)imidazo[1,2-a]pyrazin-8-amine

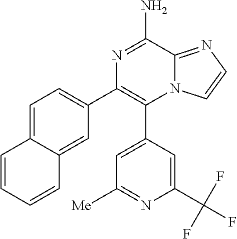 [SMILES: Cc1cc(-c2c(-c3ccc4ccccc4c3)nc(N)c3nccn23)cc(C(F)(F)F)n1]

The title compound was synthesized following the approach outlined in Procedure D, Conditions A substituting 6-chloro-5-(naphthalen-2-yl)pyrazin-2-amine [Procedure B, step (b) substituting 2-naphthylboronic acid for phenylboronic acid and heating for 5 h at 90° C.] for 6-chloro-5-phenylpyrazin-2-amine in step (a) and substituting (2-methyl-4-(tetramethyl-1,3,2-dioxaborolan-2-yl)-6-(trifluoromethyl)pyridine for 5-(tetramethyl-1,3,2-dioxaborolan-2-yl)-1H-indazole in step (d) and using the conditions A described in Procedure D heating at 150° C. for 4 h to lead to the title compound (12 mg, 10%) as a hydrochloride salt as a white solid. ESI-MS: 420.4 [M+H]+. 1H NMR (400 MHz, DMSO-d6) δ 8.99 (s, 2H), 7.98-7.96 (m, 1H), 7.94 (s, 1H), 7.93-7.92 (m, 1H), 7.92-7.89 (m, 1H), 7.89-7.86 (m, 1H), 7.86-7.83 (m, 1H), 7.74 (d, J=1.3 Hz, 1H), 7.67 (d, J=1.3 Hz, 1H), 7.60-7.56 (m, 1H), 7.56-7.52 (m, 1H), 7.36 (dd, J=8.5, 1.8 Hz, 1H), 2.52 (s, 3H).

### Example 51: 4-[8-amino-5-(3-chloro-4-hydroxyphenyl)-6-phenylimidazo[1,2-a]pyrazin-2-yl]benzonitrile

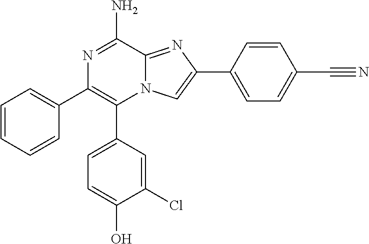 [SMILES: N#Cc1ccc(-c2cn3c(-c4ccc(O)c(Cl)c4)c(-c4ccccc4)nc(N)c3n2)cc1]

The title compound was synthesized following the approach outlined in Procedure B, Conditions B in step (c) and substituting 2-bromo-4′-cyanoacetophenone for chloroacetaldehyde in step (e) and performing this step in chloroform heating under microwave irradiation at 110° C. for 30 min in a presence of TiCl₄ (0.75 equiv.) and TEA (0.6 equiv.), then step (f1) was performed to lead to the title compound (5 mg, 8%/2 steps) as a yellow solid. ESI-MS: 438.00 [M+H]+. 1H NMR (400 MHz, DMSO-d6) δ 8.25-8.21 (m, 2H), 8.10 (s, 1H), 7.88-7.84 (m, 2H), 7.33-7.29 (m, 4H), 7.26-7.18 (m, 3H), 7.17-7.11 (m, 3H), 7.04 (d, J=8.4 Hz, 1H).

### Example 52:5-(2,6-dimethylpyridin-4-yl)-6-(3-fluorophenyl)imidazo[1,2-a]pyrazin-8-amine

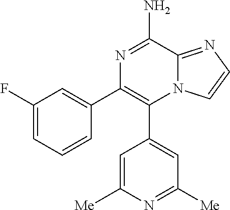 [SMILES: Cc1cc(-c2c(-c3cccc(F)c3)nc(N)c3nccn23)cc(C)n1]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 2,6-dimethyl-4-pyridylboronic acid for 5-(tetramethyl-1,3,2-dioxaborolan-2-yl)-1H-indazole in step (d) and using the conditions A described in Procedure D heating at 150° C. for 4 h to lead to the title compound (68 mg, 35%) as a hydrochloride salt as a yellow solid. ESI-MS: 334.2 [M+H]+. 1H NMR (400 MHz, DMSO-d6) δ 8.35 (s, 2H), 7.92-7.80 (m, 2H), 7.65 (s, 2H), 7.40-7.30 (m, 1H), 7.29-7.17 (m, 2H), 7.08 (d, J=7.8 Hz, 1H), 2.66 (s, 6H).

### Example 53: 4-[8-amino-6-(3-fluorophenyl)imidazo[1,2-a]pyrazin-5-yl]-2-chloro-6-methylphenol NH₂

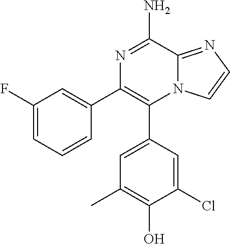 [SMILES: Cc1cc(-c2c(-c3cccc(F)c3)nc(N)c3nccn23)cc(Cl)c1O]

The title compound was synthesized following the approach outlined in Procedure B, Conditions B in step (c) substituting 3-fluorophenylboronic acid for phenylboronic acid in step (b) and substituting 2-chloro-6-methyl-4-(tetramethyl-1,3,2-dioxaborolan-2-yl)phenol for (3-chloro-4-hydroxyphenyl)-boronic acid in step (c) and performing this step for 2 h in 140° C. to afford the title compound as a hydrochloride salt (39 mg, 53.4%) as a pale yellow solid. ESI-MS: 367.00 [M+H]+. 1H NMR (300 MHz, DMSO-d6) δ 9.71 (s, 1H), 8.64 (s, 2H), 7.84 (s, 1H), 7.65 (s, 1H), 7.54-7.10 (m, 6H), 2.18 (s, 3H).

### Example 54: 4-[8-amino-6-(3,5-difluorophenyl)imidazo[1,2-a]pyrazin-5-yl]-2-chlorophenol

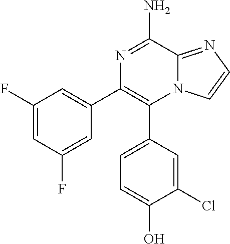 [SMILES: Nc1nc(-c2cc(F)cc(F)c2)c(-c2ccc(O)c(Cl)c2)n2ccnc12]

The title compound was synthesized following the approach outlined in Procedure B, Conditions B substituting 3,5-difluorophenylboronic acid for phenylboronic acid in step (b) and performing step (c) in 140° C. for 3 h, to afford the title compound as a hydrochloride salt (20 mg, 25%) as a white solid. ESI-MS: 373.2 [M+H]+. 1H NMR (400 MHz, DMSO-d6) δ 10.85 (s, 1H), 8.61 (s, 2H), 7.93-7.87 (m, 1H), 7.70-7.64 (m, 1H), 7.44 (d, J=2.1 Hz, 1H), 7.33-7.24 (m, 1H), 7.20 (dd, J=8.3, 2.1 Hz, 1H), 7.09 (d, J=8.4 Hz, 1H), 7.07-6.97 (m, 2H).

### Example 55:4-[8-amino-6-(3-fluorophenyl)imidazo[1,2-a]pyrazin-5-yl]-2,6-dichlorophenol

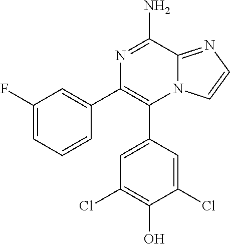 [SMILES: Nc1nc(-c2cccc(F)c2)c(-c2cc(Cl)c(O)c(Cl)c2)n2ccnc12]

The title compound was synthesized following the approach outlined in Procedure B, Conditions B in step (c) substituting 3-fluorophenylboronic acid for phenylboronic acid in step (b) and substituting 3,5-dichloro-4-methoxyphenyl boronic acid for (3-chloro-4-hydroxyphenyl)-boronic acid in step (c) to lead to the 5-(3,5-dichloro-4-methoxyphenyl)-6-(3-fluorophenyl)imidazo[1,2-a]pyrazin-8-amine which was transformed during hydrochloride salt preparation to lead to the title compound as a hydrochloride salt (37 mg, 43%) as a white solid. ESI-MS: 389.20 [M+H]+. 1H NMR (400 MHz, DMSO-d6) δ 10.67 (s, 1H), 8.12 (s, 2H), 7.70 (d, J=35.9 Hz, 2H), 7.45 (s, 2H), 7.37 (dd, J=14.2, 7.9 Hz, 1H), 7.28-7.04 (m, 3H).

### Example 56: 5-(1,3-benzothiazol-5-yl)-6-(3-fluorophenyl)imidazo[1,2-a]pyrazin-8-amine

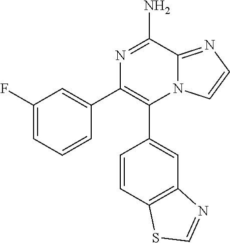 [SMILES: Nc1nc(-c2cccc(F)c2)c(-c2ccc3scnc3c2)n2ccnc12]

The title compound was synthesized following the approach outlined in Procedure D, Conditions A substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (benzothiazol-5-yl)boronic acid for 5-(tetramethyl-1,3,2-dioxaborolan-2-yl)-1H-indazole in step (d) and using the conditions A described in Procedure D heating at 150° C. for 2 h to lead to the title compound (7 mg, 9%) as a beige solid. ESI-MS: 362.0 [M+H]+. 1H NMR (400 MHz, DMSO-d6) δ 9.46 (s, 1H), 8.26 (d, J=8.4 Hz, 1H), 8.12 (d, J=1.5 Hz, 1H), 7.53 (d, J=1.2 Hz, 1H), 7.51 (dd, J=8.3, 1.7 Hz, 1H), 7.38 (d, J=1.2 Hz, 1H), 7.20-7.10 (m, 4H), 7.08-7.03 (m, 1H), 7.02-6.94 (m, 1H).

### Example 57: 4-[8-amino-6-(3-fluorophenyl)imidazo[1,2-a]pyrazin-5-yl]-2,6-dimethoxyphenol

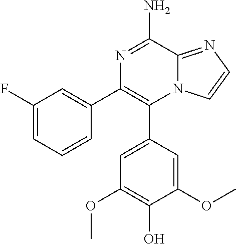 [SMILES: COc1cc(-c2c(-c3cccc(F)c3)nc(N)c3nccn23)cc(OC)c1O]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) substituting 2,6-dimethoxy-4-(tetramethyl-1,3,2-dioxaborolan-2-yl)phenol for 5-(tetramethyl-1,3,2-dioxaborolan-2-yl)-1H-indazole in step (d) and using the conditions B described in Procedure D performing this step (f) or 16 h in 150° C. to afford the title compound as a hydrochloride salt (100 mg, 56%) as a white solid. ESI-MS: 381.20 [M+H]+. 1H NMR (300 MHz, DMSO-d6) δ 8.94 (s, 2H), 7.92 (d, J=1.3 Hz, 1H), 7.83 (d, J=1.3 Hz, 1H), 7.45-7.34 (m, 1H), 7.27-7.17 (m, 3H), 6.67 (s, 2H), 3.63 (s, 6H).

### Example 58:4-[8-amino-6-(4-fluorophenyl)imidazo[1,2-a]pyrazin-5-yl]-N,N,6-trimethylpyridin-2-amine

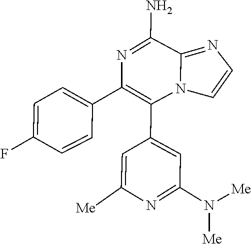 [SMILES: Cc1cc(-c2c(-c3ccc(F)cc3)nc(N)c3nccn23)cc(N(C)C)n1]

In a pressure tube was placed 5-(2-chloro-6-methylpyridin-4-yl)-6-(4-fluorophenyl)imidazo[1,2-a]pyrazin-8-amine (example 35) (50 mg, 0.14 mmol) then NMP (1 mL) was added followed by 2M dimethylamine in THE (1.43 mL, 2.8 mmol) and the reaction mixture was heated to 175° C. and stirred for 3 days. After that time the reaction mixture was cooled down to r.t., concentrated under reduced pressure and the crude material was purified by flash chromatography on silica eluting with dichloromethane/methanol and then transformed to hydrochloride salt to afford the title compound as a yellow solid (25 mg, 40%). ESI-MS: 363.2 [M+H]+. 1H NMR (300 MHz, DMSO-d6) δ 8.70 (s, 2H), 7.89 (d, J=6.7 Hz, 2H), 7.55-7.38 (m, 2H), 7.31-7.16 (m, 2H), 6.94 (s, 1H), 6.58 (s, 1H), 3.11 (s, 6H), 2.43 (s, 3H).

### Example 59:4-[8-amino-6-(4-fluorophenyl)imidazo[1,2-a]pyrazin-5-yl]-N,6-dimethylpyridin-2-amine

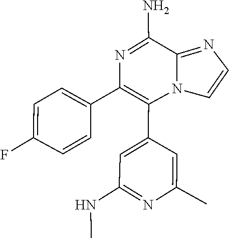 [SMILES: CNc1cc(-c2c(-c3ccc(F)cc3)nc(N)c3nccn23)cc(C)n1]

In a pressure tube was placed 5-(2-chloro-6-methylpyridin-4-yl)-6-(4-fluorophenyl)imidazo[1,2-a]pyrazin-8-amine (example 35) (50 mg, 0.14 mmol) then NMP (1 ml) was added followed by 2M methylamine in THE (0.35 ml, 0.7 mmol) and the reaction mixture was heated at 170° C. for 1 day. After that time the reaction mixture was cooled down to r.t., concentrated under reduced pressure and the crude material was purified by flash chromatography on silica eluting with dichloromethane/methanol and then transformed to hydrochloride salt to afford the title compound (17 mg, 29%) as an orange solid. ESI-MS: 349.10 [M+H]+. 1H NMR (300 MHz, DMSO-d6) δ 8.59 (s, 2H), 7.91 (d, J=17.9 Hz, 2H), 7.52-7.41 (m, 2H), 7.32-7.18 (m, 2H), 6.89 (s, 1H), 6.63 (s, 1H), 2.92 (s, 3H), 2.44 (s, 3H).

### Example 60: 6-(3-fluorophenyl)-5-(1-methyl-1H-indazol-6-yl)imidazo[1,2-a]pyrazin-8-amine

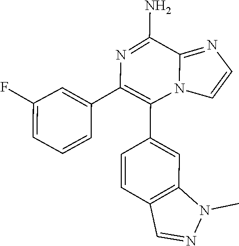 [SMILES: Cn1ncc2ccc(-c3c(-c4cccc(F)c4)nc(N)c4nccn34)cc21]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (1-methylindazol-6-yl)boronic acid for 5-(tetramethyl-1,3,2-dioxaborolan-2-yl)-1H-indazole in step (d) and using the conditions A described in Procedure D heating at 130° C. for 3 h to lead to the title compound (0.5 mg, 3%) as a white solid. ESI-MS: 359.2. 1H NMR (400 MHz, DMSO-d6) δ 8.12 (d, J=0.8 Hz, 1H), 7.84-7.79 (m, 2H), 7.53 (d, J=1.1 Hz, 1H), 7.40 (d, J=1.1 Hz, 1H), 7.16 (td, J=6.4, 1.7 Hz, 4H), 7.11-7.05 (m, 2H), 7.03-6.95 (m, 1H), 4.01 (s, 3H).

### Example 61: 6-(3-fluorophenyl)-5-(8-fluoroquinolin-6-yl)imidazo[1,2-a]pyrazin-8-amine

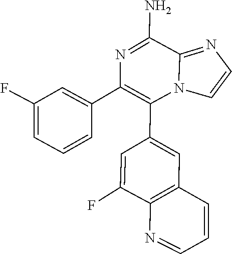 [SMILES: Nc1nc(-c2cccc(F)c2)c(-c2cc(F)c3ncccc3c2)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (8-fluoroquinolin-6-yl)boronic acid for 5-(tetramethyl-1,3,2-dioxaborolan-2-yl)-1H-indazole in step (d) and using the conditions B described in Procedure D heating at 100° C. for 3 h to lead to the title compound (70 mg, 46%) as a hydrochloride salt as a yellow solid. ESI-MS: 374.2. 1H NMR (400 MHz, DMSO-d6) δ 9.18 (s, 2H), 9.04 (dd, J=4.2, 1.6 Hz, 1H), 8.49-8.42 (m, 1H), 8.03-7.95 (m, 1H), 7.92 (d, J=1.7 Hz, 1H), 7.86 (d, J=1.4 Hz, 1H), 7.75-7.62 (m, 2H), 7.37-7.21 (m, 2H), 7.21-7.05 (m, 2H).

### Example 62: 5-(1,3-benzothiazol-6-yl)-6-(3-fluorophenyl)imidazo[1,2-a]pyrazin-8-amine

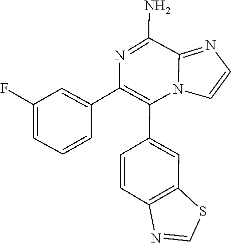 [SMILES: Nc1nc(-c2cccc(F)c2)c(-c2ccc3ncsc3c2)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (benzothiazol-6-yl)boronic acid for 5-(tetramethyl-1,3,2-dioxaborolan-2-yl)-1H-indazole in step (d) and using the conditions B described in Procedure D heating at 150° C. for 2 h to lead to the title compound (7 mg, 11%) as a hydrochloride salt as a white solid. ESI-MS: 362.5. 1H NMR (400 MHz, DMSO-d6) δ 9.52 (s, 1H), 8.30 (d, J=1.7 Hz, 1H), 8.18 (d, J=8.4 Hz, 1H), 7.88 (d, J=1.4 Hz, 1H), 7.67 (d, J=1.3 Hz, 1H), 7.56 (dd, J=8.4, 1.7 Hz, 1H), 7.37-7.27 (m, 1H), 7.23 (dt, J=9.8, 2.1 Hz, 1H), 7.19-7.09 (m, 2H).

### Example 63: 5,6-bis(1,3-benzothiazol-6-yl)imidazo[1,2-a]pyrazin-8-amine

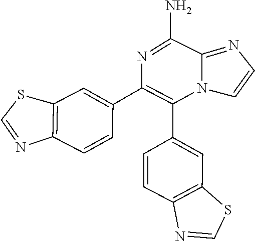 [SMILES: Nc1nc(-c2ccc3ncsc3c2)c(-c2ccc3ncsc3c2)n2ccnc12]

The title compound was synthesized following the approach outlined in Procedure E, except substituting with 2 equivalents of 6-(4,4,5,5-tetramethyl-1,3,2-dioxaborolan-2-yl)benzo[d]thiazole for 4-cyanophenylboronic acid and substituting Pd(dppf)Cl₂ for Pd(Ph₃P)₄ heating at 150° C. for 2 h at step (d), and not performing step (e) to lead to the title compound (8 mg, 2%) as a white solid. ESI-MS: 401.0. 1H NMR (400 MHz, DMSO-d6) δ 9.46 (s, 1H), 9.33 (s, 1H), 8.45 (s, 1H), 8.29 (d, J=1.6 Hz, 1H), 8.16 (d, J=1.7 Hz, 1H), 8.12 (d, J=8.4 Hz, 1H), 7.82 (d, J=8.5 Hz, 1H), 7.55 (d, J=1.2 Hz, 1H), 7.55 (dd, J=8.4, 1.7 Hz, 1H), 7.42 (d, J=1.2 Hz, 1H), 7.35 (dd, J=8.5, 1.7 Hz, 1H), 7.20 (s, 2H).

### Example 64: 6-(3-fluorophenyl)-5-(7-methyl-1H-indazol-5-yl)imidazo[1,2-a]pyrazin-8-amine

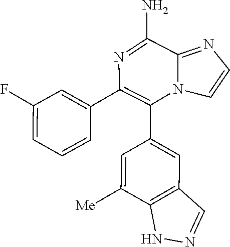 [SMILES: Cc1cc(-c2c(-c3cccc(F)c3)nc(N)c3nccn23)cc2cn[nH]c12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (7-methyl-1H-indazol-5-yl)boronic acid for 5-(tetramethyl-1,3,2-dioxaborolan-2-yl)-1H-indazole in step (d) and using the conditions A described in Procedure D heating at 150° C. for 2 h to lead to the title compound (5 mg, 5%) as a white solid. ESI-MS: 359.1. 1H NMR (400 MHz, DMSO-d6) δ 13.34 (s, 1H), 8.06 (d, J=1.4 Hz, 1H), 7.57 (s, 1H), 7.49 (d, J=1.2 Hz, 1H), 7.26 (d, J=1.3 Hz, 1H), 7.19-7.10 (m, 3H), 7.09-7.03 (m, 3H), 7.00-6.92 (m, 1H), 2.53 (s, 3H).

### Example 65:4-[8-amino-6-(3-fluoro-5-methoxyphenyl)imidazo[1,2-a]pyrazin-5-yl]-2-chlorophenol

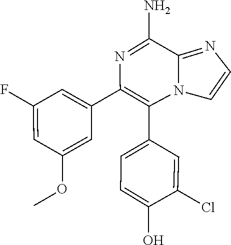 [SMILES: COc1cc(F)cc(-c2nc(N)c3nccn3c2-c2ccc(O)c(Cl)c2)c1]

The title compound was synthesized following the approach outlined in Procedure B, Conditions B in step (c), substituting 3-fluoro-5-methoxyphenylboronic acid for phenylboronic acid in step (b) and performing step (c) for 3 h in 140° C. to afford the title compound as a hydrochloride salt (46 mg, 53.2%) as a white solid. ESI-MS: 385.00 [M+H]+. 1H NMR (400 MHz, DMSO-d6) δ 10.87 (s, 1H), 9.04 (s, 2H), 7.91 (s, 1H), 7.69 (d, J=1.4 Hz, 1H), 7.45 (d, J=2.1 Hz, 1H), 7.20 (dd, J=8.3, 2.1 Hz, 1H), 7.13-7.06 (m, 1H), 6.90-6.83 (m, 1H), 6.76-6.72 (m, 2H), 3.70 (s, 3H).

### Example 66: 4-[8-amino-6-(3-fluorophenyl)imidazo[1,2-a]pyrazin-5-yl]-2,6-difluorophenol

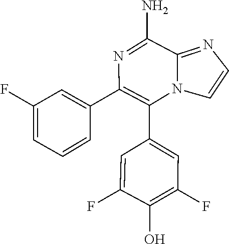 [SMILES: Nc1nc(-c2cccc(F)c2)c(-c2cc(F)c(O)c(F)c2)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (3,5-difluoro-4-hydroxyphenyl)boronic acid for 5-(tetramethyl-1,3,2-dioxaborolan-2-yl)-1H-indazole in step (d) and using the conditions A described in Procedure D heating at 140° C. for 3.5 h to lead to the title compound (30 mg, 16%) as a hydrochloride salt as a beige solid. ESI-MS: 357.1. 1H NMR (400 MHz, DMSO-d6) δ 10.85 (s, 1H), 9.07 (s, 2H), 7.96 (s, 1H), 7.87-7.79 (m, 1H), 7.46-7.38 (m, 1H), 7.27-7.20 (m, 2H), 7.18-7.13 (m, 3H).

### Example 67: ethyl 8-amino-6-(3-fluorophenyl)-5-[2-methyl-6-(trifluoromethyl)pyridin-4-yl]imidazo[1,2-a]pyrazine-2-carboxylate

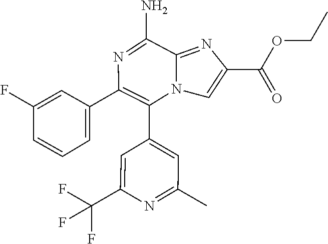 [SMILES: CCOC(=O)c1cn2c(-c3cc(C)nc(C(F)(F)F)c3)c(-c3cccc(F)c3)nc(N)c2n1]

The title compound was synthesized following the approach outlined in Procedure B, Conditions A in step (c), substituting 3-fluorophenylboronic acid for phenylboronic acid in step (b) and substituting 2-methyl-4-(tetramethyl-1,3,2-dioxaborolan-2-yl)-6-(trifluoromethyl)pyridine (Procedure A1) for (3-chloro-4-hydroxyphenyl)-boronic acid in step (c) and performing this step for 2.5 h at 150° C. and substituting 3-bromopyruvic acid ethyl ester for chloroacetaldehyde in step (e) and performing this step in DME as a solvent at 60° C. for 16 h and substituting 0.5M NH₃ in THE for ammonium in water in step (f) heating for 16 h at 90° C. Purification by HPLC (in a presence of formic acid) was done to lead to the title compound (50 mg, 28%) as a white solid. ESI-MS: 460.00 [M+H]+. 1H NMR (400 MHz, DMSO-d6) δ 8.07 (s, 1H), 7.74 (s, 1H), 7.68 (s, 2H), 7.61 (s, 1H), 7.32-7.19 (m, 1H), 7.17-7.06 (m, 2H), 7.04-6.97 (m, 1H), 4.31 (q, J=7.1 Hz, 2H), 2.56 (s, 3H), 1.29 (t, J=7.1 Hz, 3H).

### Example 68:4-[8-amino-6-(3-fluorophenyl)imidazo[1,2-a]pyrazin-5-yl]-2-chloro-6-methoxyphenol

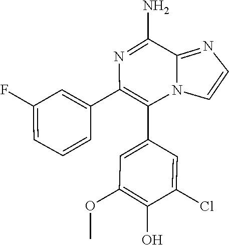 [SMILES: COc1cc(-c2c(-c3cccc(F)c3)nc(N)c3nccn23)cc(Cl)c1O]

The title compound was synthesized following the approach outlined in Procedure D substituting 6-chloro-5-(3-fluorophenyl)pyrazin-2-amine [prepared following the approach outlined in Procedure B substituting 3-fluorophenylboronic acid for phenylboronic acid in step (b)] for 6-chloro-5-phenylpyrazin-2-amine in step (a) and substituting (3-chloro-4-hydroxy-5-methoxyphenyl)boronic acid for (3-chloro-4-hydroxyphenyl)boronic acid in step (d) to lead to the title compound (5 mg, 6%) as an off-white solid. ESI-MS: 385.00 [M+H]+. 1H NMR (400 MHz, DMSO-d6) δ 9.80 (s, 1H), 7.53 (d, J=1.2 Hz, 1H), 7.48 (d, J=1.2 Hz, 1H), 7.31-7.22 (m, 1H), 7.19-7.13 (m, 1H), 7.12-7.07 (m, 3H), 7.07-7.01 (m, 1H), 7.00 (d, J=1.9 Hz, 1H), 6.93 (d, J=1.9 Hz, 1H), 3.71 (s, 3H).

### Example 69: 8-amino-6-(3-fluorophenyl)-5-[2-methyl-6-(trifluoromethyl)pyridin-4-yl]imidazo[1,2-a]pyrazine-2-carboxamide

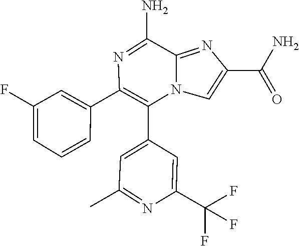 [SMILES: Cc1cc(-c2c(-c3cccc(F)c3)nc(N)c3nc(C(N)=O)cn23)cc(C(F)(F)F)n1]

The title compound was synthesized following the approach outlined in Procedure B, Conditions A in step (c) and substituting 3-fluorophenylboronic acid for phenylboronic acid in step (b) and substituting 2-methyl-4-(tetramethyl-1,3,2-dioxaborolan-2-yl)-6-(trifluoromethyl)pyridine (Procedure A1) for (3-chloro-4-hydroxyphenyl)-boronic acid in step (c) and performing this step (f) or 2.5 h at 150° C. and substituting 3-bromopyruvic acid ethyl ester for chloroacetaldehyde in step (e) and performing this step in DME as a solvent at 60° C. for 16 h and performing step (f) in a mixture of 0.5M NH₃ in dioxane/28% NH₃ in water 5:2 for 4 days at 140° C. to lead to the title compound (4 mg, 28%) as a white solid. ESI-MS: 431.00 [M+H]+. 1H NMR (400 MHz, DMSO-d6) δ 7.96 (s, 1H), 7.75 (s, 1H), 7.62 (d, J=1.3 Hz, 1H), 7.53-7.50 (m, 2H), 7.47 (s, 2H), 7.31-7.23 (m, 1H), 7.17-7.07 (m, 2H), 7.04-6.98 (m, 1H), 2.57 (s, 3H).

### Example 70:6-(3-fluorophenyl)-5-(2-methylpyridin-4-yl)imidazo[1,2-a]pyrazin-8-amine

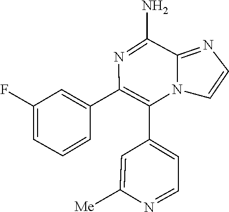 [SMILES: Cc1cc(-c2c(-c3cccc(F)c3)nc(N)c3nccn23)ccn1]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (2-methylpyridin-4-yl)boronic acid for 5-(tetramethyl-1,3,2-dioxaborolan-2-yl)-1H-indazole in step (d) and using the conditions B described in Procedure D heating at 100° C. for 2 h to lead to the title compound (110 mg, 81%) as a hydrochloride salt as a yellow solid. ESI-MS: 320.1. 1H NMR (400 MHz, DMSO-d6) δ 9.25-8.75 (s, 2H), 8.75 (d, J=6.0 Hz, 1H), 8.08-8.01 (m, 1H), 8.00 (dd, J=4.8, 1.6 Hz, 2H), 7.66 (dd, J=6.0, 1.7 Hz, 1H), 7.42-7.33 (m, 1H), 7.31-7.19 (m, 2H), 7.10 (dt, J=7.7, 1.3 Hz, 1H), 2.75 (s, 3H).

### Example 71: 8-amino-5-(3-chloro-4-hydroxyphenyl)-6-(3-fluorophenyl)imidazo[1,2-a]pyrazine-2-carboxylic Acid

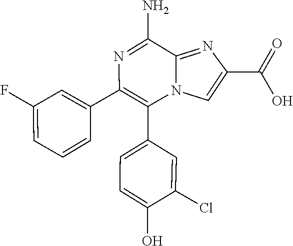 [SMILES: Nc1nc(-c2cccc(F)c2)c(-c2ccc(O)c(Cl)c2)n2cc(C(=O)O)nc12]

The title compound was synthesized following the approach outlined in Procedure B, Conditions B in step (c), substituting 3-fluorophenylboronic acid for phenylboronic acid in step (b) and substituting 3-bromopyruvic acid ethyl ester for chloroacetaldehyde in step (e) and performing this step in DME as a solvent, at 60° C. for 16 h and performing step (f) at 90° C. for 16 h. Purification by HPLC (with presence of formic acid) to lead to the title compound (17 mg, 29%) as a white solid. ESI-MS: 399.00 [M+H]+. 1H NMR (400 MHz, DMSO-d6) δ 12.89 (s, 1H), 10.67 (s, 1H), 7.66 (s, 1H), 7.43 (d, J=2.1 Hz, 1H), 7.39 (s, 2H), 7.27 (td, J=8.0, 6.3 Hz, 1H), 7.20 (dd, J=8.4, 2.1 Hz, 1H), 7.14 (dt, J=10.6, 2.0 Hz, 1H), 7.10 (d, J=8.0 Hz, 1H), 7.05 (d, J=8.3 Hz, 2H).

### Example 72:5-(2,6-dichloropyridin-4-yl)-6-(3-fluorophenyl)imidazo[1,2-a]pyrazin-8-amine

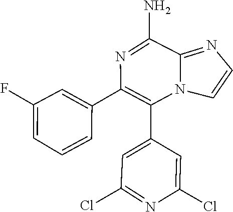 [SMILES: Nc1nc(-c2cccc(F)c2)c(-c2cc(Cl)nc(Cl)c2)n2ccnc12]

The title compound was synthesized following the approach outlined in Procedure B, Conditions A in step (c) substituting 3-fluorophenylboronic acid for phenylboronic acid in step (b) and substituting Pd(amphos)Cl₂ for Pd(PPh3)4 and 2,6-dichloropyridine-4-boronic acid for (3-chloro-4-hydroxyphenyl)-boronic acid in step (c) and performing this reaction in dioxane for 0.5 h in 100° C. to lead to the title compound as a hydrochloride salt (37 mg, 97%) as a white solid. ESI-MS: 374.10 [M+H]+. 1H NMR (400 MHz, DMSO-d6) δ 8.48 (s, 2H), 7.94 (s, 1H), 7.87 (s, 1H), 7.65 (s, 2H), 7.44-7.34 (m, 1H), 7.24 (d, J=9.6 Hz, 2H), 7.09 (d, J=7.6 Hz, 1H).

### Example 73:4-[8-amino-6-(3-fluorophenyl)imidazo[1,2-a]pyrazin-5-yl]-N,6-dimethylpyridin-2-amine

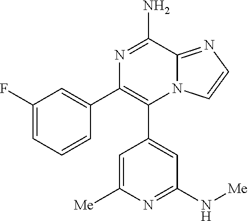 [SMILES: CNc1cc(-c2c(-c3cccc(F)c3)nc(N)c3nccn23)cc(C)n1]

In a pressure tube was placed 5-(2-chloro-6-methylpyridin-4-yl)-6-(3-fluorophenyl)imidazo[1,2-a]pyrazin-8-amine (example 34) (52 mg, 0.15 mmol) then NMP (0.5 mL) was added followed by 2M methylamine in THE (3 mL, 6 mmol) and the reaction mixture was warmed to 170° C. and stirred for 14 days. After that time the reaction mixture was cooled down to r.t., concentrated under reduced pressure and the crude material was purified by flash chromatography on silica eluting with dichloromethane/methanol and then transformed to hydrochloride salt to afford the title compound as a orange solid (29 mg, 51%). ESI-MS: 349.1 [M+H]+. 1H NMR (400 MHz, DMSO-d6) δ 8.39 (s, 2H), 7.94-7.89 (m, 1H), 7.85 (s, 1H), 7.44-7.37 (m, 1H), 7.31-7.12 (m, 3H), 6.90 (s, 1H), 6.66 (s, 1H), 2.91 (s, 3H), 2.44 (s, 3H).

### Example 74: 6-(3-fluorophenyl)-5-{imidazo[1,2-a]pyridin-6-yl}imidazo[1,2-a]pyrazin-8-amine

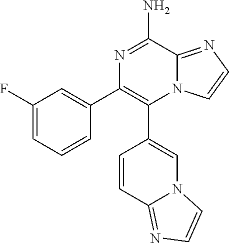 [SMILES: Nc1nc(-c2cccc(F)c2)c(-c2ccc3nccn3c2)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting imidazo[1,2-a]pyridin-6-ylboronic acid for 5-(tetramethyl-1,3,2-dioxaborolan-2-yl)-1H-indazole in step (d) and using the conditions A described in Procedure D heating at 150° C. for 4.5 h to lead to the title compound (21 mg, 9%) as a hydrochloride salt as a yellow solid. ESI-MS: 345.1. 1H NMR (400 MHz, DMSO-d6) δ 9.15-9.08 (m, 1H), 8.70 (s, 1H), 8.42 (d, J=1.8 Hz, 1H), 8.27 (d, J=2.1 Hz, 1H), 8.08 (d, J=9.3 Hz, 1H), 7.92 (d, J=5.9 Hz, 2H), 7.86 (dd, J=9.3, 1.5 Hz, 1H), 7.40-7.26 (m, 2H), 7.24-7.13 (m, 2H).

### Example: 75: 4-[8-amino-6-(3-fluorophenyl)imidazo[1,2-a]pyrazin-5-yl]-2,6-dimethylphenol

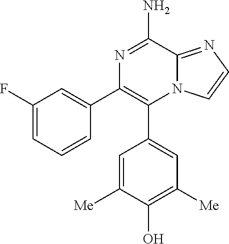 [SMILES: Cc1cc(-c2c(-c3cccc(F)c3)nc(N)c3nccn23)cc(C)c1O]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (4-hydroxy-3,5-dimethylphenyl)boronic acid for 5-(tetramethyl-1,3,2-dioxaborolan-2-yl)-1H-indazole in step (d) and using the conditions B described in Procedure D heating at 150° C. for 2 h to lead to the title compound (25 mg, 12%) as a hydrochloride salt as a white solid. ESI-MS: 349.1. 1H NMR (400 MHz, DMSO-d6) δ 8.75 (s, 1H), 8.52 (s, 2H), 7.80 (s, 1H), 7.56 (s, 1H), 7.41-7.28 (m, 1H), 7.25-7.10 (m 3H), 6.96 (s, 2H), 2.13 (s, 6H).

### Example 76:8-amino-6-(3-fluorophenyl)-5-[2-methyl-6-(trifluoromethyl)pyridin-4-yl]imidazo[1,2-a]pyrazine-2-carboxylic Acid

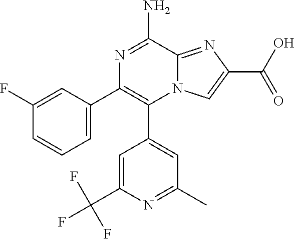 [SMILES: Cc1cc(-c2c(-c3cccc(F)c3)nc(N)c3nc(C(=O)O)cn23)cc(C(F)(F)F)n1]

The title compound was synthesized following the approach outlined in Procedure B, Conditions A in step (c) substituting 3-fluorophenylboronic acid for phenylboronic acid in step (b) and substituting 2-methyl-4-(tetramethyl-1,3,2-dioxaborolan-2-yl)-6-(trifluoromethyl)pyridine (Procedure A1) for (3-chloro-4-hydroxyphenyl)-boronic acid in step (c) and performing this step (f) or 2.5 h at 150° C. and substituting 3-bromopyruvic acid ethyl ester for chloroacetaldehyde in step (e) and performing this step in DME as a solvent heating at 60° C. for 16 h. Purification by HPLC (in a presence of formic acid) was done to lead to the title compound (5 mg, 11%) as a white solid. ESI-MS: 432.00 [M+H]⁺. ¹H NMR (400 MHz, DMSO-d6) δ 12.98 (s, 1H), 7.95 (s, 1H), 7.74 (s, 1H), 7.63 (s, 2H), 7.61 (s, 1H), 7.27 (td, J=7.9, 6.0 Hz, 1H), 7.18-7.05 (m, 2H), 7.01 (d, J=7.9 Hz, 1H), 2.56 (s, 3H).

### Example 77:8-amino-6-(3-fluorophenyl)-N,N-dimethyl-5-[2-methyl-6-(trifluoromethyl)pyridin-4-yl]imidazo[1,2-a]pyrazine-2-carboxamide

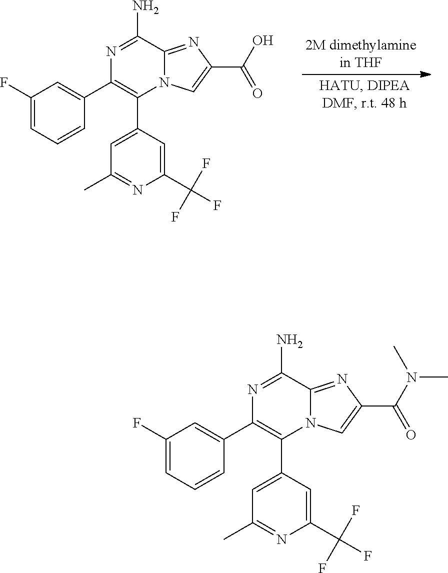 [SMILES: Cc1cc(-c2c(-c3cccc(F)c3)nc(N)c3nc(C(=O)O)cn23)cc(C(F)(F)F)n1 ; Cc1cc(-c2c(-c3cccc(F)c3)nc(N)c3nc(C(=O)N(C)C)cn23)cc(C(F)(F)F)n1]

In a dry flask was placed 8-amino-6-(3-fluorophenyl)-5-[2-methyl-6-(trifluoromethyl)pyridin-4-yl]imidazo[1,2-a]pyrazine-2-carboxylic acid (example 76) (30 mg, 0.07 mmol) then DMF (2 mL) was added and to this HATU (31 mg, 0.08 mmol, 1.2 equiv.) was added. Reaction mixture was stirred for 10 minutes and then DIPEA (0.04 mL, 0.21 mmol, 3 equiv.) followed by 2M dimethylamine solution in THE (0.04 mL, 0.08 mmol, 1.1 equiv.) were added. Reaction was stirred at r.t. for 2 d. Reaction mixture was concentrated under reduced pressure and purified by using flash chromatography (silica gel, DCM/MeOH 0% to 10%) to lead to the title compound as light yellow solid as a hydrochloride salt (8 mg, 24%). ESI-MS: 459.00 [M+H]+. 1H NMR (400 MHz, DMSO-d6) δ 8.03 (s, 2H), 7.92 (s, 1H), 7.71 (d, J=1.3 Hz, 1H), 7.60 (d, J=1.3 Hz, 1H), 7.36-7.26 (m, 1H), 7.22-7.12 (m, 2H), 7.10-7.01 (m, 1H), 3.35 (s, 3H), 3.00 (s, 3H), 2.55 (s, 3H).

### Example 78: 8-amino-6-(3-fluorophenyl)-N-methyl-5-[2-methyl-6-(trifluoromethyl)pyridin-4-yl]imidazo[1,2-a]pyrazine-2-carboxamide

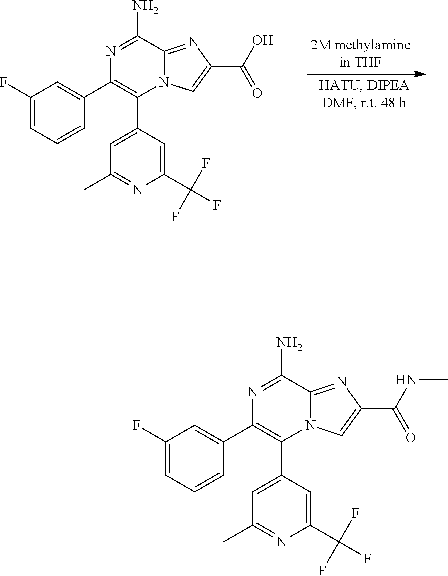 [SMILES: Cc1cc(-c2c(-c3cccc(F)c3)nc(N)c3nc(C(=O)O)cn23)cc(C(F)(F)F)n1 ; CNC(=O)c1cn2c(-c3cc(C)nc(C(F)(F)F)c3)c(-c3cccc(F)c3)nc(N)c2n1]

In a dry flask was placed 8-amino-6-(3-fluorophenyl)-5-[2-methyl-6-(trifluoromethyl)pyridin-4-yl]imidazo[1,2-a]pyrazine-2-carboxylic acid (example 76) (30 mg, 0.07 mmol) then DMF (2 mL) was added and to this HATU (31 mg, 0.08 mmol, 1.2 equiv.) was added. Reaction mixture was stirred for 10 minutes and then DIPEA (0.04 mL, 0.21 mmol, 3 equiv.) followed by 2M methylamine solution in THE (0.04 mL, 0.08 mmol, 1.1 equiv.) were added. Reaction was stirred at r.t. for 2 d. Reaction mixture was concentrated under reduced pressure and purified by using flash chromatography (silica gel, DCM/MeOH 0% to 10%) to lead to the title compound as light yellow solid, hydrochloride salt (10 mg, 27%) as a light yellow solid. ESI-MS: 445.10 [M+H]+. 1H NMR (400 MHz, DMSO-d6) δ 8.16 (d, J=5.1 Hz, 1H), 7.98 (s, 1H), 7.75 (s, 1H), 7.70 (s, 2H), 7.62 (s, 1H), 7.36-7.25 (m, 1H), 7.23-7.06 (m, 2H), 7.03 (dd, J=7.7, 1.3 Hz, 1H), 2.80 (d, J=4.7 Hz, 3H), 2.57 (s, 3H).

### Example 79:5-(4-amino-3,5-dichlorophenyl)-6-(3-fluorophenyl)imidazo[1,2-a]pyrazin-8-amine

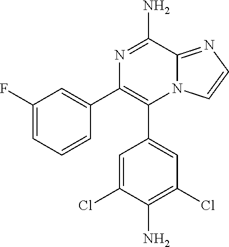 [SMILES: Nc1c(Cl)cc(-c2c(-c3cccc(F)c3)nc(N)c3nccn23)cc1Cl]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 2,6-dichloro-4-(4,4,5,5-tetramethyl-1,3,2-dioxaborolan-2-yl)aniline for 5-(tetramethyl-1,3,2-dioxaborolan-2-yl)-1H-indazole in step (d) and using the conditions B described in Procedure D heating at 110° C. for 2 h to lead to the title compound (37 mg, 20%) as a hydrochloride salt as a yellow solid. ESI-MS: 388.0. 1H NMR (400 MHz, DMSO-d6) δ 8.63 (s, 2H), 7.85 (s, 1H), 7.73 (s, 1H), 7.46-7.36 (m, 1H), 7.31 (s, 2H), 7.27-7.11 (m, 3H), 5.94 (s, 2H).

### Example 80: 6-(3-fluorophenyl)-5-(isoquinolin-6-yl)imidazo[1,2-a]pyrazin-8-amine

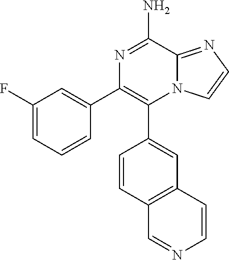 [SMILES: Nc1nc(-c2cccc(F)c2)c(-c2ccc3cnccc3c2)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (isoquinolin-6-yl)boronic acid for 5-(tetramethyl-1,3,2-dioxaborolan-2-yl)-1H-indazole in step (d) and using the conditions D described in Procedure B heating at 100° C. for 2 h to lead to the title compound (46 mg, 30%) as a hydrochloride salt as a yellow solid. ESI-MS: 356.1 [M+H]+. 1H NMR (400 MHz, DMSO-d6) δ 9.87 (s, 1H), 9.18-8.76 (m, 2H), 8.71 (d, J=6.3 Hz, 1H), 8.53 (d, J=8.6 Hz, 1H), 8.44-8.35 (m, 2H), 7.95 (s, 1H), 7.88 (dd, J=8.6, 1.5 Hz, 1H), 7.78 (s, 1H), 7.33-7.26 (m, 1H), 7.26-7.12 (m, 1H), 7.12-7.07 (m, 1H).

### Example 81:6-(3-fluorophenyl)-5-(2-methoxy-6-methylpyridin-4-yl)imidazo[1,2-a]pyrazin-8-amine

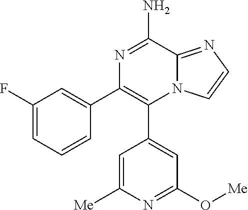 [SMILES: COc1cc(-c2c(-c3cccc(F)c3)nc(N)c3nccn23)cc(C)n1]

In a dry flask was placed anhydrous methanol (3 mL) and all was cooled to 0° C. Then sodium (68 mg, 3 mmol) was added and after 15 min a solution of 5-(2-fluoro-6-methylpyridin-4-yl)-6-(3-fluorophenyl)imidazo[1,2-a]pyrazin-8-amine (example 46) (250 mg, 0.7 mmol) in MeOH (2 mL) was added. Reaction mixture was then heated in reflux for 1 h. After that time the reaction mixture was cooled down to r.t. then diluted with water. Aqueous layer was extracted with EtOAc. Separated organic layer was dried over anhydrous sodium sulfate, filtered and concentrated under reduced pressure which was purified by flash chromatography on silica eluting with hexane/ethyl acetate. Title product was obtained as a hydrochloride salt as a white solid (5 mg, 19%). ESI-MS: 350.1 [M+H]+. 1H NMR (400 MHz, Methanol-d4) δ 9.14 (d, J=1.3 Hz, 1H), 9.07 (d, J=1.3 Hz, 1H), 8.84-8.76 (m, 1H), 8.75-8.65 (m, 2H), 8.61-8.53 (m, 1H), 8.39 (s, 1H), 8.17-8.11 (m, 1H), 5.44 (s, 3H), 3.97 (s, 3H).

### Example 82: 5-(1H-1,3-benzodiazol-6-yl)-6-(3-fluorophenyl)imidazo[1,2-a]pyrazin-8-amine

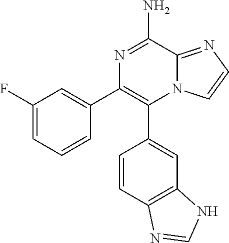 [SMILES: Nc1nc(-c2cccc(F)c2)c(-c2ccc3nc[nH]c3c2)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (1H-benzimidazol-6-yl)boronic acid for 5-(tetramethyl-1,3,2-dioxaborolan-2-yl)-1H-indazole in step (d) and using the conditions A described in Procedure D to lead to the title compound (62 mg, 88%) as a hydrochloride salt as a brown solid. ESI-MS: 345.1 [M+H]+. 1H NMR (400 MHz, DMSO-d6) δ 9.53 (s, 1H), 8.87 (s, 2H), 7.99-7.92 (m, 2H), 7.92-7.87 (m, 1H), 7.61 (d, J=1.2 Hz, 1H), 7.58 (dd, J=8.5, 1.5 Hz, 1H), 7.36-7.27 (m, 1H), 7.27-7.19 (m, 1H), 7.19-7.10 (m, 2H).

### Example 83: 6-(3-fluorophenyl)-5-(1-methyl-1H-1,3-benzodiazol-6-yl)imidazo[1,2-a]pyrazin-8-amine

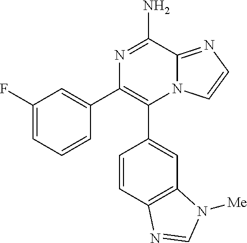 [SMILES: Cn1cnc2ccc(-c3c(-c4cccc(F)c4)nc(N)c4nccn34)cc21]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 1-methyl-1H-benzimidazol-6-yl)boronic acid for 5-(tetramethyl-1,3,2-dioxaborolan-2-yl)-1H-indazole in step (d) and using the conditions A described in Procedure B heating at 150° C. for 3 h to lead to the title compound (22 mg, 15%) as a hydrochloride salt as a white solid. ESI-MS: 359.1 [M+H]+. 1H NMR (400 MHz, DMSO-d6) δ 9.50 (s, 1H), 8.88 (s, 2H), 8.17 (s, 1H), 7.96-7.88 (m, 2H), 7.65 (d, J=1.3 Hz, 1H), 7.48 (dd, J=8.5, 1.4 Hz, 1H), 7.35-7.27 (m, 1H), 7.27-7.22 (m, 1H), 7.20-7.13 (m, 2H), 4.02 (s, 3H (superimposed on water signal)).

### Example 84: ethyl 8-amino-5-(3-chloro-4-hydroxyphenyl)-6-(3-fluorophenyl)imidazo[1,2-a]pyrazine-2-carboxylate

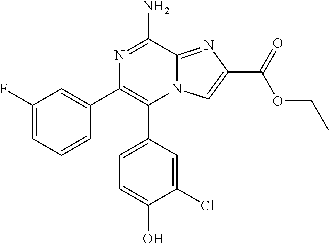 [SMILES: CCOC(=O)c1cn2c(-c3ccc(O)c(Cl)c3)c(-c3cccc(F)c3)nc(N)c2n1]

The title compound was synthesized following the approach outlined in Procedure B, Conditions B in step (c), substituting 3-fluorophenylboronic acid for phenylboronic acid in step (b) and substituting 3-bromopyruvic acid ethyl ester for chloroacetaldehyde in step (e) and performing this step in DME as a solvent heating at 60° C. for 16 h and 0.5M NH₃ in THE was substituted for ammonium in water in step (f). Crude reaction mixture was purified by HPLC (with presence of formic acid) to lead to the title compound (6 mg, 8%) as a white solid. ESI-MS: 427.10 [M+H]+. 1H NMR (400 MHz, DMSO-d6) δ 7.70 (s, 1H), 7.44 (s, 2H), 7.41 (d, J=2.0 Hz, 1H), 7.26 (q, J=7.9 Hz, 1H), 7.19 (dd, J=8.3, 2.0 Hz, 1H), 7.13 (d, J=10.2 Hz, 1H), 7.06 (dd, J=16.2, 8.1 Hz, 3H), 4.29 (q, J=7.1 Hz, 2H), 1.28 (t, J=7.1 Hz, 3H).

### Example 85:4-[8-amino-6-(4-fluorophenyl)imidazo[1,2-a]pyrazin-5-yl]-6-methyl-N-(propan-2-yl)pyridin-2-amine

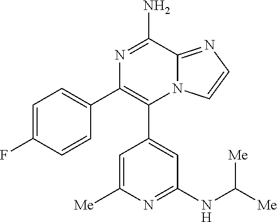 [SMILES: Cc1cc(-c2c(-c3ccc(F)cc3)nc(N)c3nccn23)cc(NC(C)C)n1]

In a pressure tube was placed 5-(2-chloro-6-methylpyridin-4-yl)-6-(4-fluorophenyl)imidazo[1,2-a]pyrazin-8-amine (example 35) (50 mg, 0.14 mmol) then NMP (0.5 mL) was added followed by isopropylamine (0.11 mL, 1.3 mmol) and the reaction mixture was warmed to 175° C. and stirred for 5 days. After that time the reaction mixture was cooled down to r.t., concentrated under reduced pressure and the crude material was purified by flash chromatography on silica eluting with hexane/ethyl acetate and then transformed to hydrochloride salt to afford the title compound as a yellow solid (7 mg, 13%). ESI-MS: 377.2 [M+H]+. 1H NMR (300 MHz, DMSO-d6) δ 7.85 (s, 1H), 7.75 (s, 1H), 7.50-7.40 (m, 2H), 7.27-7.16 (m, 2H), 6.79 (s, 1H), 6.71 (s, 1H), 2.43 (s, 3H), 1.26-1.22 (m, 1H), 1.11 (d, J=6.3 Hz, 6H).

### Example 86: 6-(3-fluorophenyl)-5-(4-methyl-1,3-benzothiazol-6-yl)imidazo[1,2-a]pyrazin-8-amine

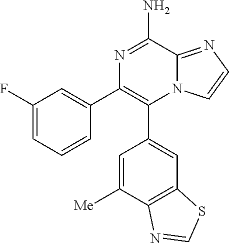 [SMILES: Cc1cc(-c2c(-c3cccc(F)c3)nc(N)c3nccn23)cc2scnc12]

A) (4-Methyl-1,3-benzothiazol-6-yl)boronic acid was prepared following the approach outlined in Procedure A2, substituting 6-bromo-4-methyl-1,3-benzothiazole for 5-bromo-1H-indazole and using 1,4-dioxane for DMF and heating for 1 h at 80° C. The reaction mixture was filtered through Celite® and the filtrate was concentrated under reduced pressure to give a red solid (quant.) which was used in next step without further purification. ESI-MS: 193.8 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (4-methyl-1,3-benzothiazol-6-yl)boronic acid for 5-(tetramethyl-1,3,2-dioxaborolan-2-yl)-1H-indazole in step (d) and using the conditions B described in Procedure B to lead to the title compound (113 mg, 79%) as a hydrochloride salt as a white solid. ESI-MS: 376.1 [M+H]+. 1H NMR (400 MHz, DMSO-d6) δ 9.48 (s, 1H), 9.21 (s, 2H), 8.08 (s, 1H), 7.97-7.88 (m, 1H), 7.69 (d, J=1.4 Hz, 1H), 7.45-7.39 (m, 1H), 7.37-7.28 (m, 1H), 7.28-7.21 (m, 1H), 7.21-7.10 (m, 2H), 2.67 (s, 3H).

### Example 87: 8-amino-6-(3-fluorophenyl)-5-[2-methyl-6-(trifluoromethyl)pyridin-4-yl]-N-(oxolan-3-yl)imidazo[1,2-a]pyrazine-2-carboxamide

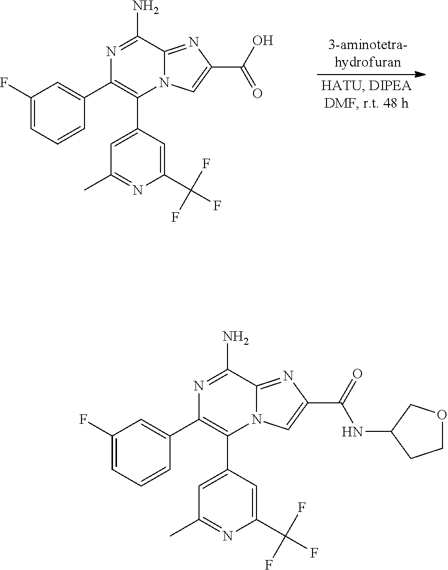 [SMILES: Cc1cc(-c2c(-c3cccc(F)c3)nc(N)c3nc(C(=O)O)cn23)cc(C(F)(F)F)n1 ; Cc1cc(-c2c(-c3cccc(F)c3)nc(N)c3nc(C(=O)NC4CCOC4)cn23)cc(C(F)(F)F)n1]

In a dry flask was placed 8-amino-6-(3-fluorophenyl)-5-[2-methyl-6-(trifluoromethyl)pyridin-4-yl]imidazo[1,2-a]pyrazine-2-carboxylic acid (example 76) (30 mg, 0.07 mmol) then DMF (2 mL) was added and to this HATU (31 mg, 0.08 mmol, 1.2 equiv.) was added. Reaction mixture was stirred for 10 minutes and then DIPEA (0.04 mL, 0.21 mmol, 3 equiv.) followed by 3-aminotetrahydrofuran (0.06 mg, 0.07 mmol, 1 equiv.) were added and reaction was stirred at r.t. for 48 h. Reaction mixture was concentrated under reduced pressure and purified by using flash chromatography (silica gel, DCM/MeOH 0% to 10%) to lead to the title compound as a off-white solid, hydrochloride salt (10 mg, 29%). ESI-MS: 501.20 [M+H]+. 1H NMR (400 MHz, DMSO-d6) δ 8.20 (d, J=7.2 Hz, 1H), 8.05 (s, 1H), 7.88 (s, 2H), 7.76 (s, 1H), 7.62 (s, 1H), 7.35-7.25 (m, 1H), 7.20-7.11 (m, 2H), 7.08-7.00 (m, 1H), 4.54-4.42 (m, 1H), 3.91-3.79 (m, 2H), 3.75-3.68 (m, 2H), 2.58 (s, 3H), 2.25-2.15 (m, 1H), 1.95-1.85 (m, 1H).

### Example 88: 5-(8-chloroquinolin-6-yl)-6-(3-fluorophenyl)imidazo[1,2-a]pyrazin-8-amine

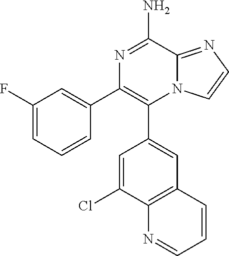 [SMILES: Nc1nc(-c2cccc(F)c2)c(-c2cc(Cl)c3ncccc3c2)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (8-chloroquinolin-6-yl)boronic acid for 5-(tetramethyl-1,3,2-dioxaborolan-2-yl)-1H-indazole in step (d) and using the conditions B described in Procedure D heating at 100° C. for 20 h to lead to the title compound (35 mg, 21%) as a hydrochloride salt as a yellow solid. ESI-MS: 390.1 [M+H]+. 1H NMR (400 MHz, DMSO-d6) δ 9.11 (dd, J=4.2, 1.7 Hz, 1H+NH2), 8.49 (dd, J=8.4, 1.7 Hz, 1H), 8.12 (d, J=1.8 Hz, 1H), 7.99 (d, J=1.8 Hz, 1H), 7.94 (d, J=1.4 Hz, 1H), 7.85 (d, J=1.4 Hz, 1H), 7.72 (dd, J=8.3, 4.2 Hz, 1H), 7.38-7.23 (m, 2H), 7.22-7.09 (m, 2H).

### Example 89:4-[8-amino-6-(3-fluorophenyl)-2-(4-methylpiperazine-1-carbonyl)imidazo[1,2-a]pyrazin-5-yl]-2-chlorophenol

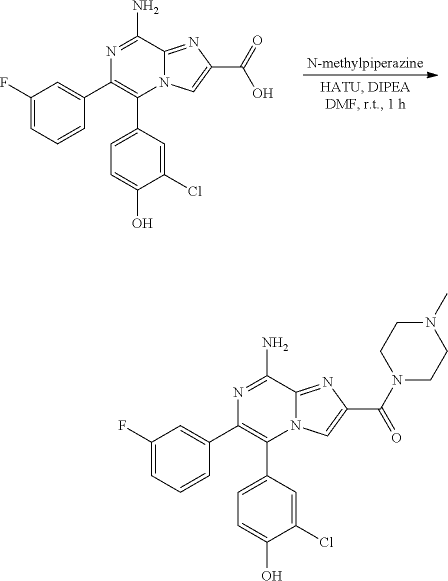 [SMILES: Nc1nc(-c2cccc(F)c2)c(-c2ccc(O)c(Cl)c2)n2cc(C(=O)O)nc12 ; CN1CCN(C(=O)c2cn3c(-c4ccc(O)c(Cl)c4)c(-c4cccc(F)c4)nc(N)c3n2)CC1]

In a dry flask was placed 8-amino-5-(3-chloro-4-hydroxyphenyl)-6-(3-fluorophenyl)imidazo[1,2-a]pyrazine-2-carboxylic acid (example 71) (10 mg, 0.03 mmol) then DMF (1 mL) was added and to this HATU (11 mg, 0.03 mmol, 1.2 equiv.) was added. Reaction mixture was stirred for 10 minutes and then DIPEA (0.013 mL, 0.08 mmol, 3 equiv.) and N-methylpiperazine (0.028 mL, 0.03 mmol, 1 equiv.) was added. Reaction was stirred at r.t. for 1 h. Reaction mixture was concentrated under reduced pressure and purified by using flash chromatography (silica gel, DCM/MeOH 0% to 10%) to lead to the title compound as a hydrochloride salt (20 mg, 41%) as a white solid. ESI-MS: 481.20 [M+H]+. 1H NMR (400 MHz, Methanol-d4) δ 8.31 (s, 1H), 7.82 (s, 1H), 7.34 (d, J=2.1 Hz, 1H), 7.25 (m, 1H), 7.17-7.10 (m, 3H), 7.03-6.96 (m, 2H), 4.50 (s, 2H), 3.90 (s, 2H), 2.90 (m, 4H), 2.60 (s, 3H).

### Example 90: 6-[8-amino-6-(3-fluorophenyl)imidazo[1,2-a]pyrazin-5-yl]-1-methyl-2,3-dihydro-1H-1,3-benzodiazol-2-one

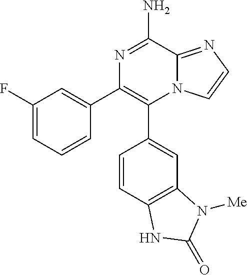 [SMILES: Cn1c(=O)[nH]c2ccc(-c3c(-c4cccc(F)c4)nc(N)c4nccn34)cc21]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 1-methyl-6-(tetramethyl-1,3,2-dioxaborolan-2-yl)-2,3-dihydro-1H-1,3-benzodiazol-2-one for 5-(tetramethyl-1,3,2-dioxaborolan-2-yl)-1H-indazole in step (d) and using the conditions A described in Procedure D heating at 150° C. for 3 h to lead to the title compound (7 mg, 6%) as a hydrochloride salt as a yellow solid. ESI-MS: 375.1 [M+H]+. 1H NMR (400 MHz, DMSO-d6) δ 11.12 (s, 1H), 8.61 (s, 2H), 7.90-7.80 (m, 1H), 7.71-7.60 (m, 1H), 7.42-7.30 (m, 1H), 7.28-7.21 (m, 2H), 7.20-7.13 (m, 2H), 7.07-6.99 (m, 1H), 6.95 (dd, J=8.0, 1.5 Hz, 1H), 3.24 (s, 3H).

### Example 91: 5-[8-amino-6-(3-fluorophenyl)imidazo[1,2-a]pyrazin-5-yl]-2,3-dihydro-1H-indol-2-one

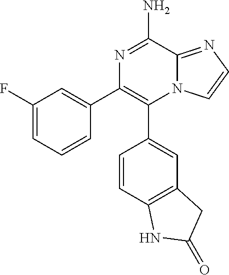 [SMILES: Nc1nc(-c2cccc(F)c2)c(-c2ccc3c(c2)CC(=O)N3)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (2,3-dihydro-2-oxo-1H-indol-5-yl)boronic acid for 5-(tetramethyl-1,3,2-dioxaborolan-2-yl)-1H-indazole in step (d) and using the conditions A described in Procedure D heating at 150° C. for 3 h to lead to the title compound (9 mg, 6%) as a hydrochloride salt as a yellow solid. ESI-MS: 360.1 [M+H]+. 1H NMR (400 MHz, DMSO-d6) δ 10.62 (s, 1H), 8.55 (s, 2H), 7.86-7.80 (m, 1H), 7.60-7.55 (m, 1H), 7.39-7.32 (m, 1H), 7.30 (s, 1H), 7.24-7.14 (m, 4H), 6.88 (d, J=8.0 Hz, 1H), 3.51 (s, 2H).

### Example 92: 6-(3-fluorophenyl)-5-(quinoxalin-6-yl)imidazo[1,2-a]pyrazin-8-amine

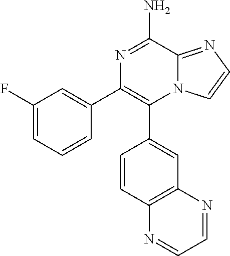 [SMILES: Nc1nc(-c2cccc(F)c2)c(-c2ccc3nccnc3c2)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (quinoxalin-6-yl)boronic acid for 5-(tetramethyl-1,3,2-dioxaborolan-2-yl)-1H-indazole in step (d) and using the conditions B described in Procedure D heating at 140° C. for 4 h to lead to the title compound (113 mg, 31%) as a hydrochloride salt as a yellow solid. ESI-MS: 357.1 [M+H]+. 1H NMR (400 MHz, DMSO-d6) δ 9.03 (d, J=1.8 Hz, 1H), 9.00 (d, J=1.8 Hz, 1H), 8.80 (s, 1H), 8.24-8.18 (m, 2H), 7.90 (d, J=1.3 Hz, 1H), 7.87 (dd, J=8.6, 1.9 Hz, 1H), 7.75 (d, J=1.3 Hz, 1H), 7.34-7.22 (m, 2H), 7.19-7.10 (m, 2H).

### Example 93: 5-(2-chloropyridin-4-yl)-6-(3-fluorophenyl)imidazo[1,2-a]pyrazin-8-amine

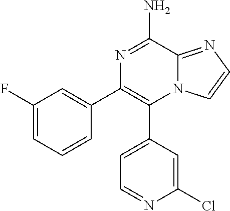 [SMILES: Nc1nc(-c2cccc(F)c2)c(-c2ccnc(Cl)c2)n2ccnc12]

The title compound was synthesized following the approach outlined in Procedure B, Conditions B substituting 3-fluorophenylboronic acid for phenylboronic acid in step (b) and heating the reaction for 20 h and substituting (2-chloropyridin-4-yl)boronic acid for (3-chloro-4-hydroxyphenyl)-boronic acid in step (c) and substituting Pd(amphos)Cl₂ for Pd(dppf)Cl₂ performing the reaction at 140° C. for 3 h to afford the title compound as a hydrochloride salt as a white solid (6 mg, 8%). ESI-MS: 340.0 [M+H]+. 1H NMR (400 MHz, DMSO-d6) δ 8.56-8.45 (m, 1H), 8.07 (s, 2H), 7.80-7.68 (m, 2H), 7.64-7.55 (m, 1H), 7.45 (dd, J=5.1, 1.4 Hz, 1H), 7.39-7.30 (m, 1H), 7.26-7.12 (m, 2H), 7.13-7.04 (m, 1H).

### Example 94:5-(4-fluoro-1,3-benzothiazol-6-yl)-6-(3-fluorophenyl)imidazo[1,2-a]pyrazin-8-amine

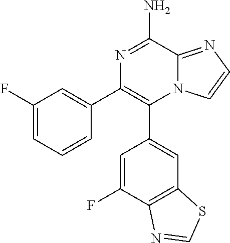 [SMILES: Nc1nc(-c2cccc(F)c2)c(-c2cc(F)c3ncsc3c2)n2ccnc12]

A) (4-Fluoro-1,3-benzothiazol-6-yl)boronic acid was prepared following the approach outlined in Procedure A2, substituting 6-Bromo-4-fluorobenzothiazole for 5-bromo-1H-indazole and using 1,4-dioxane for DMF and heating for 2 h at 80° C. The reaction mixture was filtered through Celite® and the filtrate was concentrated under reduced pressure to give a yellow solid (quant.) which was used in next step without further purification. ESI-MS: 198.0 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (4-fluoro-1,3-benzothiazol-6-yl)boronic acid for 5-(tetramethyl-1,3,2-dioxaborolan-2-yl)-1H-indazole in step (d) and using the conditions B described in Procedure D heating at 100° C. for 18 h to lead to the title compound (128 mg, 40%) as a hydrochloride salt as a white solid. ESI-MS: 380.1 [M+H]+. 1H NMR (400 MHz, DMSO-d6) δ 9.54 (s, 1H), 8.47 (s, 2H), 8.10 (d, J=1.4 Hz, 1H), 7.83-7.80 (m, 1H), 7.73-7.69 (m, 1H), 7.54 (dd, J=11.1, 1.5 Hz, 1H), 7.34-7.27 (m, 1H), 7.27-7.22 (m, 1H), 7.17-7.10 (m, 2H).

### Example 95: 5-[8-amino-6-(3-fluorophenyl)imidazo[1,2-a]pyrazin-5-yl]-3-bromopyridin-2-ol

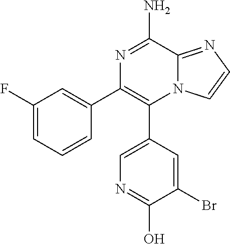 [SMILES: Nc1nc(-c2cccc(F)c2)c(-c2cnc(O)c(Br)c2)n2ccnc12]

The title compound was synthesized following the approach outlined in Procedure B, Conditions B substituting 3-fluorophenylboronic acid for phenylboronic acid in step (b) and heating the reaction for 20 h and substituting 5-(4,4,5,5-tetramethyl-1,3,2-dioxaborolan-2-yl)pyridin-2-ol for (3-chloro-4-hydroxyphenyl)-boronic acid in step (c) and performing the reaction at 130° C. for 3 h and using NBS in 2 equiv. in step (d) to afford the title compound as a beige solid (2 mg, 3%). ESI-MS: 400.1 [M+H]+. 1H NMR (400 MHz, Methanol-d4) δ 9.61 (d, J=2.3 Hz, 1H), 9.19 (d, J=1.2 Hz, 1H), 9.17 (d, J=1.2 Hz, 1H), 9.00 (d, J=2.4 Hz, 1H), 8.94-8.86 (m, 1H), 8.81-8.75 (m, 1H), 8.75-8.71 (m, 1H), 8.64-8.58 (m, 1H).

### Example 96: 5-[8-amino-6-(3-fluorophenyl)imidazo[1,2-a]pyrazin-5-yl]-1,2-dihydropyridin-2-one

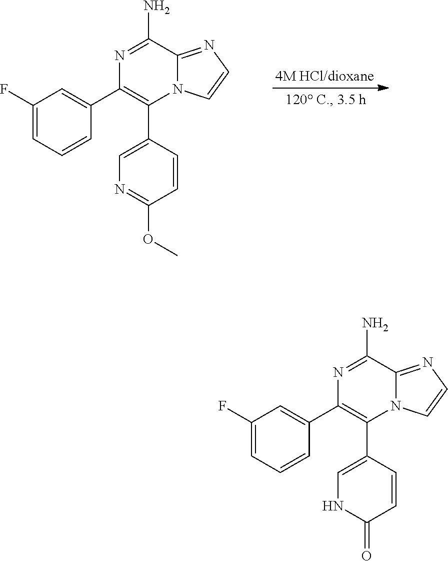 [SMILES: COc1ccc(-c2c(-c3cccc(F)c3)nc(N)c3nccn23)cn1 ; Nc1nc(-c2cccc(F)c2)c(-c2ccc(=O)[nH]c2)n2ccnc12]

Step 1) 6-(3-Fluorophenyl)-5-(6-methoxypyridin-3-yl)imidazo[1,2-a]pyrazin-8-amine was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (6-methoxypyridin-3-yl)-boronic acid for 5-(tetramethyl-1,3,2-dioxaborolan-2-yl)-1H-indazole in step (d) and using the conditions A described in Procedure D to lead to the product (25 mg, 76%) as a beige solid. ESI-MS: 336.2 [M+H]+. 1H NMR (400 MHz, DMSO-d6) δ 8.10 (dd, J=2.4, 0.8 Hz, 1H), 7.83 (dd, J=8.5, 2.5 Hz, 1H), 7.55 (d, J=1.2 Hz, 1H), 7.41 (d, J=1.2 Hz, 1H), 7.31-7.22 (m, 1H), 7.17 (s, 2H), 7.15-7.10 (m, 1H), 7.09-7.01 (m, 2H), 6.94 (dd, J=8.5, 0.7 Hz, 1H), 3.88 (s, 3H).

Step 2) In a pressure tube was suspended 6-(3-fluorophenyl)-5-(6-methoxypyridin-3-yl)imidazo[1,2-a]pyrazin-8-amine (100 mg, 0.3 mmol) in 4M HCl in dioxane (6 mL) and heated at 120° C. for 3.5 h. After cooling to room temperature, crude reaction mixture was triturated with diethyl ether followed by pentane, precipitates were collected. Then solids were dissolved in hot EtOH and triturated with diethyl ether followed by pentane once more, macerated with diethyl ether and dried under reduced pressure. The title product 5-[8-amino-6-(3-fluorophenyl)imidazo[1,2-a]pyrazin-5-yl]-1,2-dihydropyridin-2-one was obtained as a hydrochloride salt as a white solid (95 mg, 85%). ESI-MS: 322.1 [M+H]+. 1H NMR (300 MHz, DMSO-d6) δ 9.02 (s, 2H), 8.03-7.87 (m, 2H), 7.55-7.35 (m, 3H), 7.34-7.18 (m, 3H), 6.42 (d, J=9.4 Hz, 1H).

### Example 97:8-amino-5-(3-chloro-4-hydroxyphenyl)-6-(3-fluorophenyl)imidazo[1,2-a]pyrazine-2-carboxamide

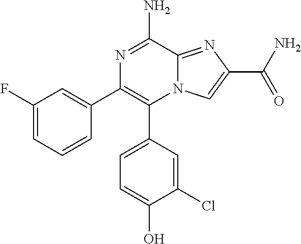 [SMILES: NC(=O)c1cn2c(-c3ccc(O)c(Cl)c3)c(-c3cccc(F)c3)nc(N)c2n1]

The title compound was synthesized following the approach outlined in Procedure B, Conditions B in step (c) substituting 3-fluorophenylboronic acid for phenylboronic acid in step (b) and substituting 3-bromopyruvic acid ethyl ester for chloroacetaldehyde in step (e) and performing this step in DME as a solvent heating at 60° C. for 16 h. Purification by HPLC (in a presence of formic acid) was done to lead to the title compound (5 mg, 19%) as an off-white solid. ESI-MS: 398.10 [M+H]+. 1H NMR (400 MHz, DMSO-d6) δ 10.64 (s, 1H), 7.70 (s, 1H), 7.55 (s, 1H), 7.47 (s, 1H), 7.43 (d, J=2.1 Hz, 1H), 7.32-7.17 (m, 4H), 7.14 (dt, J=10.6, 2.1 Hz, 1H), 7.07 (dd, J=20.1, 8.3 Hz, 3H).

### Example 98: 6-(3-fluorophenyl)-5-[2-methyl-6-(trifluoromethyl)pyridin-4-yl]-2-phenylimidazo[1,2-a]pyrazin-8-amine

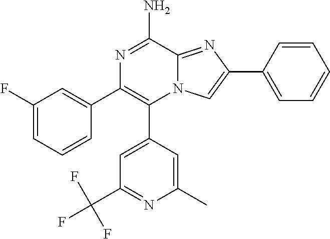 [SMILES: Cc1cc(-c2c(-c3cccc(F)c3)nc(N)c3nc(-c4ccccc4)cn23)cc(C(F)(F)F)n1]

The title compound was synthesized following the approach outlined in Procedure B, Conditions B in step (c) substituting 3-fluorophenylboronic acid for phenylboronic acid in step (b) and substituting 2-methyl-4-(tetramethyl-1,3,2-dioxaborolan-2-yl)-6-(trifluoromethyl)pyridine (Procedure A1) for (3-chloro-4-hydroxyphenyl)-boronic acid in step (c) and substituting 2-bromoacetophenone for chloroacetaldehyde in step (e) and performing this step in acetonitrile as a solvent at 130° C. for 3 days to lead to the title compound as a hydrochloride salt (6 mg, 38%) as a yellow solid. ESI-MS: 464.2 [M+H]+. 1H NMR (300 MHz, DMSO-d6) δ 8.20 (s, 1H), 8.10-8.01 (m, 2H), 7.79 (s, 1H), 7.61 (d, J=1.3 Hz, 1H), 7.48-7.41 (m, 2H), 7.38-7.26 (m, 2H), 7.20-7.00 (m, 3H), 2.60 (s, 3H).

### Example 99:5-[8-amino-6-(3-fluorophenyl)imidazo[1,2-a]pyrazin-5-yl]-1,3-dimethyl-1,2-dihydropyridin-2-one

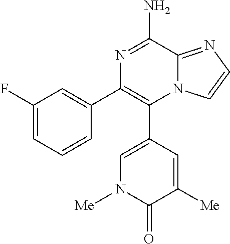 [SMILES: Cc1cc(-c2c(-c3cccc(F)c3)nc(N)c3nccn23)cn(C)c1=O]

The title compound was synthesized following the approach outlined in Procedure B, Conditions B in step (c) substituting 3-fluorophenylboronic acid for phenylboronic acid in step (b) and substituting 1,3-dimethyl-5-(4,4,5,5-tetramethyl-1,3,2-dioxaborolan-2-yl)pyridin-2-one for (3-chloro-4-hydroxyphenyl)-boronic acid in step (c) to lead to the title compound as a hydrochloride salt (5 mg, 13%) as a beige solid. ESI-MS: 350.1 [M+H]+. 1H NMR (400 MHz, Acetonitrile-d3) δ 8.75 (s, 2H), 7.85 (d, J=1.4 Hz, 1H), 7.74 (d, J=1.5 Hz, 1H), 7.45-7.36 (m, 2H), 7.30-7.24 (m, 2H), 7.21-7.13 (m, 2H), 3.39 (s, 3H), 2.03 (s, 3H).

### Example 100:6-(3-fluorophenyl)-5-[2-(pyrrolidin-1-yl)pyridin-4-yl]imidazo[1,2-a]pyrazin-8-amine

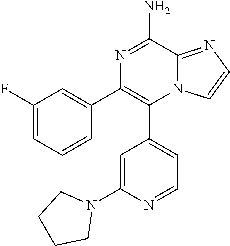 [SMILES: Nc1nc(-c2cccc(F)c2)c(-c2ccnc(N3CCCC3)c2)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting [2-(pyrrolidin-1-yl)pyridin-4-yl]boronic acid for 5-(tetramethyl-1,3,2-dioxaborolan-2-yl)-1H-indazole in step (d) and using the conditions B described in Procedure D heating at 100° C. for 3 h to lead to the title compound (106 mg, 67%) as a hydrochloride salt as a yellow solid. ESI-MS: 375.2 [M+H]+. 1H NMR (400 MHz, DMSO-d6) δ 8.87 (s, 2H), 8.11-8.07 (m, 1H), 8.02-7.98 (m, 1H), 7.96 (d, J=6.5 Hz, 1H), 7.47-7.38 (m, 1H), 7.37-7.19 (m, 4H), 6.64 (dd, J=6.5, 1.4 Hz, 1H), 3.63-3.42 (m, 4H), 2.09-1.94 (m, 4H).

### Example 101:4-[8-amino-2-(aminomethyl)-6-phenylimidazo[1,2-a]pyrazin-5-yl]-2-chlorophenol

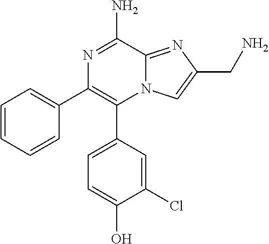 [SMILES: NCc1cn2c(-c3ccc(O)c(Cl)c3)c(-c3ccccc3)nc(N)c2n1]

The title compound was synthesized following the approach outlined in Procedure B, Conditions B in step (c) substituting 1,3-dichloroacetone for chloroacetaldehyde in step (e) and performing this step in acetonitrile as a solvent at 100° C. for 16 h and to lead to the title compound as a hydrochloride salt (3 mg, 10.3%) as a dark grey solid. ESI-MS: 366.10 [M+H]+. 1H NMR (400 MHz, DMSO-d6) δ 10.67 (s, 2H), 8.19 (s, 2H), 7.52 (s, 1H), 7.38 (d, J=2.1 Hz, 1H), 7.36-7.30 (m, 2H), 7.28-7.19 (m, 3H), 7.10 (dd, J=8.3, 2.1 Hz, 1H), 7.04 (d, J=8.4 Hz, 2H), 4.12 (s, 2H).

### Example 102: 6-(3-fluorophenyl)-5-{pyrazolo[1,5-a]pyrimidin-6-yl}imidazo[1,2-a]pyrazin-8-amine

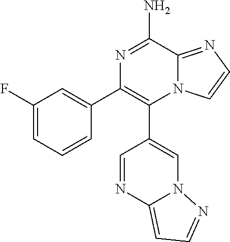 [SMILES: Nc1nc(-c2cccc(F)c2)c(-c2cnc3ccnn3c2)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 6-(4,4,5,5-Tetramethyl-1,3,2-dioxaborolan-2-yl)pyrazolo[1,5-a]pyrimidine for 5-(tetramethyl-1,3,2-dioxaborolan-2-yl)-1H-indazole in step (d) and using the conditions A described in Procedure D to lead to the title compound (4 mg, 6%) as a hydrochloride salt as a light yellow solid. ESI-MS: 346.0. 1H NMR (400 MHz, DMSO-d6) δ 9.35 (dd, J=2.1, 1.0 Hz, 1H), 8.45 (d, J=2.1 Hz, 1H), 8.31 (d, J=2.4 Hz, 1H), 7.98 (s, 1H), 7.76 (s, 1H), 7.37-7.24 (m, 2H), 7.21-7.08 (m, 2H), 6.80 (dd, J=2.4, 0.9 Hz, 1H).

### Example 103: 6-(3-fluorophenyl)-5-(8-methylquinolin-6-yl)imidazo[1,2-a]pyrazin-8-amine

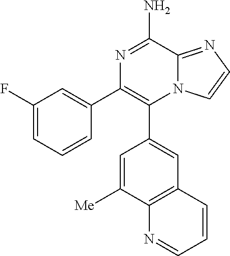 [SMILES: Cc1cc(-c2c(-c3cccc(F)c3)nc(N)c3nccn23)cc2cccnc12]

A) (8-Methylquinolin-6-yl)boronic acid was prepared following the approach outlined in Procedure A2, substituting 6-chloro-8-methylquinoline for 5-bromo-1H-indazole and using 1,4-dioxane for DMF and heating for 18 h at 80° C. to give, after flash chromatography, a white solid (quant.) which was used in next step. ESI-MS: 187.9 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (8-methylquinolin-6-yl)boronic acid for 5-(tetramethyl-1,3,2-dioxaborolan-2-yl)-1H-indazole in step (d) and using the conditions B described in Procedure D heating at 150° C. for 2 h to lead to the title compound (7 mg, 4%) as a hydrochloride salt as a green solid. ESI-MS: 370.1 [M+H]+. 1H NMR (400 MHz, DMSO-d6) δ 9.03 (dd, J=4.3, 1.8 Hz, 1H), 8.78 (s, 2H), 8.40 (dd, J=8.4, 1.8 Hz, 1H), 7.93 (d, J=1.9 Hz, 1H), 7.88 (s, 1H), 7.74-7.55 (m, 3H), 7.39-7.22 (m, 2H), 7.22-7.04 (m, 2H), 2.71 (s, 3H).

### Example 104: 6-(4-fluorophenyl)-5-(8-fluoroquinolin-6-yl)imidazo[1,2-a]pyrazin-8-amine

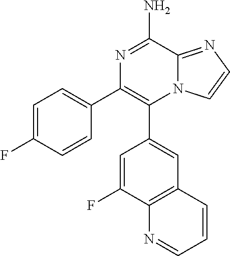 [SMILES: Nc1nc(-c2ccc(F)cc2)c(-c2cc(F)c3ncccc3c2)n2ccnc12]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (8-fluoroquinolin-6-yl)boronic acid for 5-(tetramethyl-1,3,2-dioxaborolan-2-yl)-1H-indazole in step (d) and using the conditions B described in Procedure D heating at 140° C. for 3 h to lead to the title compound (29 mg, 37%) as a hydrochloride salt as a yellow solid. ESI-MS: 374.1 [M+H]+. 1H NMR (400 MHz, DMSO-d6) δ 9.03 (dd, J=4.2, 1.6 Hz, 1H), 8.46-8.40 (m, 1H), 8.21 (s, 1H), 7.91-7.87 (m, 1H), 7.77-7.73 (m, 1H), 7.72-7.70 (m, 1H), 7.70-7.63 (m, 2H), 7.44-7.36 (m 2H), 7.16-7.08 (m 2H).

### Example 105: 6-(4-fluorophenyl)-5-(1-methyl-1H-1,2,3-benzotriazol-6-yl)imidazo[1,2-a]pyrazin-8-amine

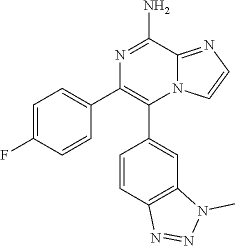 [SMILES: Cn1nnc2ccc(-c3c(-c4ccc(F)cc4)nc(N)c4nccn34)cc21]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting 1-methyl-6-(4,4,5,5-tetramethyl-1,3,2-dioxaborolan-2-yl)-1H-benzotriazole for 5-(tetramethyl-1,3,2-dioxaborolan-2-yl)-1H-indazole in step (d) and using the conditions A described in Procedure D heating at 130° C. for 3 h to lead to the title compound (10 mg, 4.85%) as a hydrochloride salt as a yellow solid. ESI-MS: 360.1 [M+H]+. 1H NMR (400 MHz, DMSO-d6) δ 8.11-8.07 (m, 2H), 7.86 (s, 1H), 7.73-7.68 (m, 1H), 7.43-7.38 (m, 2H), 7.28 (dd, J=8.7, 1.4 Hz, 1H), 7.18-7.11 (m, 2H), 4.30 (s, 3H).

### Example 106: 6-[8-amino-6-(3-fluorophenyl)imidazo[1,2-a]pyrazin-5-yl]-1,3-benzothiazol-2-amine

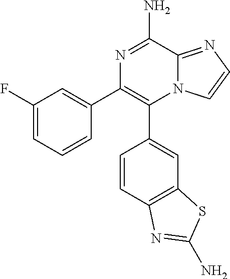 [SMILES: Nc1nc2ccc(-c3c(-c4cccc(F)c4)nc(N)c4nccn34)cc2s1]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (2-aminobenzothiazol-6-yl)boronic acid for 5-(tetramethyl-1,3,2-dioxaborolan-2-yl)-1H-indazole in step (d) and using the conditions B described in Procedure D heating at 140° C. for 3 h to lead to the title compound (74 mg, 10%) as a hydrochloride salt as a white solid. ESI-MS: 377.1 [M+H]+. 1H NMR (400 MHz, DMSO-d6) δ 8.83 (s, 4H), 7.94-7.89 (m, 1H), 7.85 (d, J=1.7 Hz, 1H), 7.66 (d, J=1.3 Hz, 1H), 7.49 (d, J=8.3 Hz, 1H), 7.40-7.30 (m, 2H), 7.26-7.18 (m, 2H), 7.18-7.11 (m, 1H).

### Example 107: 6-(3-fluorophenyl)-5-(8-fluoroquinoxalin-6-yl)imidazo[1,2-a]pyrazin-8-amine

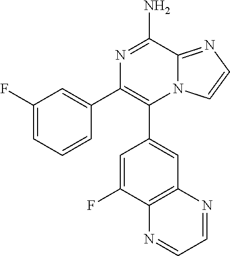 [SMILES: Nc1nc(-c2cccc(F)c2)c(-c2cc(F)c3nccnc3c2)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 8-fluoroquinoxalin-6-ylboronic acid for 5-(tetramethyl-1,3,2-dioxaborolan-2-yl)-1H-indazole in step (d) and using the conditions B described in Procedure D heating at 120° C. for 5 h to lead to the title compound (16 mg, 20%) as a hydrochloride salt as a yellow solid. ESI-MS: 375.1 [M+H]+. 1H NMR (400 MHz, DMSO-d6) δ 9.07 (s, 2H), 8.03-7.98 (m, 1H), 7.85 (dd, J=10.6, 1.7 Hz, 1H), 7.77 (dd, J=10.5, 1.3 Hz, 2H), 7.35-7.22 (m, 2H), 7.18-7.04 (m, 2H).

### Example 108: 6-(3-fluorophenyl)-5-{8-methylimidazo[1,2-a]pyridin-6-yl}imidazo[1,2-a]pyrazin-8-amine

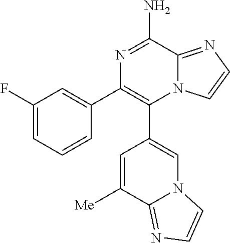 [SMILES: Cc1cc(-c2c(-c3cccc(F)c3)nc(N)c3nccn23)cn2ccnc12]

A) {8-methylimidazo[1,2-a]pyridin-6-yl}boronic acid was prepared following the approach outlined in Procedure A2, substituting 6-Bromo-8-methylimidazo[1,2-a]pyridine for 5-bromo-1H-indazole and using 1,4-dioxane for DMF and heating for 18 h at 80° C. The reaction mixture was filtered through Celite® and the filtrate was concentrated under reduced pressure to give a brown solid (quant.) which was used in next step without further purification. ESI-MS: 177.0 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting {8-methylimidazo[1,2-a]pyridin-6-yl}boronic acid for 5-(tetramethyl-1,3,2-dioxaborolan-2-yl)-1H-indazole in step (d) and using the conditions B described in Procedure D heating at 120° C. for 5 h to lead to the title compound (13 mg, 25%) as a hydrochloride salt as a beige solid. ESI-MS: 359.1 [M+H]+. 1H NMR (400 MHz, DMSO-d6) δ 8.89 (d, J=1.4 Hz, 1H), 8.38 (d, J=2.1 Hz, 1H), 8.28 (d, J=2.1 Hz, 1H), 8.22 (s, 2H) 7.79 (d, J=3.8 Hz, 2H), 7.75 (d, J=1.3 Hz, 1H), 7.39-7.25 (m, 2H), 7.25-7.03 (m, 2H), 2.60 (s, 3H).

### Example 109: 3-[8-amino-5-(8-fluoroquinolin-6-yl)imidazo[1,2-a]pyrazin-6-yl]benzonitrile

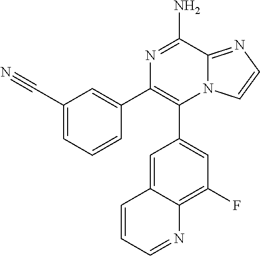 [SMILES: N#Cc1cccc(-c2nc(N)c3nccn3c2-c2cc(F)c3ncccc3c2)c1]

The title compound was synthesized following the approach outlined in Procedure B, Conditions A in step (c), substituting 3-cyanophenylboronic acid for phenylboronic acid in step (b) and substituting Pd(amphos)Cl₂ for Pd(PPh₃) in step (c) and 8-fluoro-6-(tetramethyl-1,3,2-dioxaborolan-2-yl)quinoline for (3-chloro-4-hydroxyphenyl)-boronic acid in step (c) and performing this reaction for 3 h in 140° C. to lead to the title compound as a hydrochloride salt (85 mg, 55%) as a yellow solid. ESI-MS: 381.10 [M+H]+. 1H NMR (400 MHz, DMSO-d6) δ 9.03 (dd, J=4.2, 1.5 Hz, 1H), 8.42 (d, J=8.4 Hz, 1H), 7.93 (s, 2H), 7.92-7.89 (m, 1H), 7.88-7.85 (m, 1H), 7.74-7.71 (m, 1H), 7.71-7.67 (m, 4H), 7.58-7.53 (m, 1H), 7.41 (t, J=7.8 Hz, 1H).

### Example 110: N-{4-[8-amino-6-(3-fluorophenyl)imidazo[1,2-a]pyrazin-5-yl]-6-methylpyridin-2-yl}acetamide

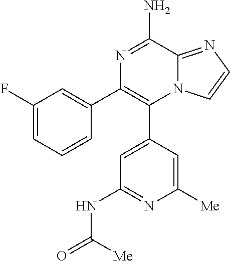 [SMILES: CC(=O)Nc1cc(-c2c(-c3cccc(F)c3)nc(N)c3nccn23)cc(C)n1]

A) (2-Acetamido-6-methylpyridin-4-yl)boronic acid was synthesized following the approach outlined in Procedure A1 substituting 2-acetamido-6-methylpyridine for 2-trifluoromethyl-6-methyl pyridine heating at 50° C. for 6 h to afford the title compound as a brown solid (quant.). ESI-MS: 195.00 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (2-acetamido-6-methylpyridin-4-yl)boronic acid for 5-(tetramethyl-1,3,2-dioxaborolan-2-yl)-1H-indazole in step (d) and using the conditions B described in Procedure D heating at 140° C. for 3 h to lead to the title compound (103 mg, 24%) as a hydrochloride salt as a yellow solid. ESI-MS: 377.1 [M+H]+. 1H NMR (400 MHz, DMSO-d6) δ 10.63 (s, 1H), 8.20 (s, 1H), 7.93 (s, 1H), 7.74 (d, J=1.2 Hz, 1H), 7.65 (d, J=1.3 Hz, 1H), 7.39-7.31 (m, 1H), 7.24-7.17 (m, 2H), 7.17-7.11 (m, 1H), 6.98-6.96 (m, 1H), 2.38 (s, 3H), 2.05 (s, 3H).

### Example 111:6-(3-fluorophenyl)-5-[8-(trifluoromethyl)quinolin-6-yl]imidazo[1,2-a]pyrazin-8-amine

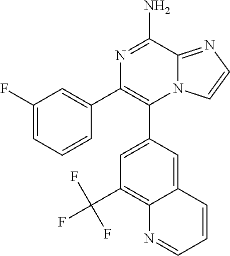 [SMILES: Nc1nc(-c2cccc(F)c2)c(-c2cc(C(F)(F)F)c3ncccc3c2)n2ccnc12]

The title compound was synthesized following the approach outlined in Procedure B, Conditions B in step (c) substituting 3-fluorophenylboronic acid for phenylboronic acid in step (b) and substituting 6-(tetramethyl-1,3,2-dioxaborolan-2-yl)-8-(trifluoromethyl)quinoline for (3-chloro-4-hydroxyphenyl)-boronic acid in step (c) and performing this step (f) or 3 h in 130° C. to lead to the title compound as a hydrochloride salt (50 mg, 17%) as a yellow solid. ESI-MS: 424.10 [M+H]+. 1H NMR (300 MHz, Methanol-d4) δ 9.07 (dd, J=4.3, 1.7 Hz, 1H), 8.43 (dd, J=8.4, 1.7 Hz, 1H), 8.39 (s, 1H), 8.12 (s, 1H), 7.83 (d, J=1.1 Hz, 1H), 7.74 (d, J=1.1 Hz, 1H), 7.70 (dd, J=8.4, 4.3 Hz, 1H), 7.39-7.08 (m, 4H).

### Example 112: 6-(3-fluorophenyl)-5-(8-methoxyquinolin-6-yl)imidazo[1,2-a]pyrazin-8-amine

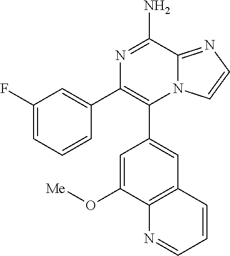 [SMILES: COc1cc(-c2c(-c3cccc(F)c3)nc(N)c3nccn23)cc2cccnc12]

A) (8-Methoxyquinolin-6-yl)boronic acid was prepared following the approach outlined in Procedure A2, substituting 6-bromo-8-methoxyquinoline for 5-bromo-1H-indazole and using 1,4-dioxane for DMF and heating for 16 h at 120° C. The reaction mixture was filtered through Celite® and the filtrate was concentrated under reduced pressure to give a brown solid (quant.) which was used in next step without further purification. ESI-MS: 203.8 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (8-methoxyquinolin-6-yl)boronic acid for 5-(tetramethyl-1,3,2-dioxaborolan-2-yl)-1H-indazole in step (d) and using the conditions A described in Procedure D heating at 130° C. for 3 h to lead to the title compound (10 mg, 22%) as a hydrochloride salt as a yellow solid. ESI-MS: 386.2 [M+H]+. 1H NMR (400 MHz, Acetonitrile-d3) δ 9.16 (dd, J=5.2, 1.3 Hz, 1H), 8.92-8.82 (m, 1H), 8.51 (s, 2H), 8.01 (dd, J=8.4, 5.2 Hz, 1H), 7.82 (d, J=1.4 Hz, 1H), 7.77 (d, J=1.4 Hz, 1H), 7.59 (d, J=1.4 Hz, 1H), 7.42 (d, J=1.3 Hz, 1H), 7.30-7.21 (m, 2H), 7.19-7.14 (m, 1H), 7.12-7.04 (m, 1H), 4.02 (s, 3H).

### Example 113: 6-(3-fluorophenyl)-5-(1,8-naphthyridin-3-yl)imidazo[1,2-a]pyrazin-8-amine

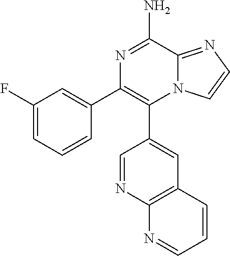 [SMILES: Nc1nc(-c2cccc(F)c2)c(-c2cnc3ncccc3c2)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 1,8-naphthyridin-3-ylboronic acid for 5-(tetramethyl-1,3,2-dioxaborolan-2-yl)-1H-indazole in step (d) and using the conditions B described in Procedure D heating at 100° C. for 20 h to lead to the title compound (24 mg, 13%) as a hydrochloride salt as a yellow solid. ESI-MS: 357.1 [M+H]+. 1H NMR (400 MHz, DMSO-d6) δ 9.20 (dd, J=4.3, 2.0 Hz, 1H), 8.94 (d, J=2.4 Hz, 1H), 8.76 (d, J=2.4 Hz, 1H), 8.62 (dd, J=8.2, 2.0 Hz, 1H), 7.95 (s, 2H), 7.78 (dd, J=8.2, 4.3 Hz, 1H), 7.39-7.23 (m, 2H), 7.22-7.09 (m, 2H).

### Example 114: 6-(3-fluorophenyl)-5-(7-fluoroquinolin-6-yl)imidazo[1,2-a]pyrazin-8-amine

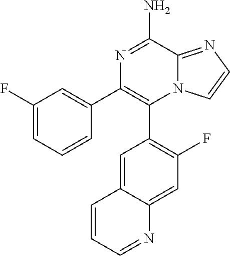 [SMILES: Nc1nc(-c2cccc(F)c2)c(-c2cc3cccnc3cc2F)n2ccnc12]

A) (7-Fluoroquinolin-6-yl)boronic acid was prepared following the approach outlined in Procedure A2, substituting 6-Bromo-7-fluoroquinoline for 5-bromo-1H-indazole and using 1,4-dioxane for DMF and heating for 1 h at 80° C. The reaction mixture was filtered through Celite® and the filtrate was concentrated under reduced pressure to give a red solid (quant.) which was used in next step without further purification. ESI-MS: 192.0 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (7-fluoroquinolin-6-yl)boronic acid for 5-(tetramethyl-1,3,2-dioxaborolan-2-yl)-1H-indazole in step (d) and using the conditions A described in Procedure D to lead to the title compound (15 mg, 5%) as a hydrochloride salt as a brown solid. ESI-MS: 374.1 [M+H]+. 1H NMR (400 MHz, DMSO-d6) δ 9.04 (dd, J=4.3, 1.6 Hz, 1H), 8.48-8.45 (m, 1H), 8.23 (d, J=7.9 Hz, 1H), 7.98 (d, J=10.9 Hz, 1H), 7.86-7.81 (m, 1H), 7.75 (s, 1H), 7.66-7.60 (m, 1H), 7.34-7.23 (m, 2H), 7.19-7.09 (m, 2H).

### Example 115: 5-(4-fluoro-1-methyl-1H-1,3-benzodiazol-6-yl)-6-(3-fluorophenyl)imidazo[1,2-a]pyrazin-8-amine

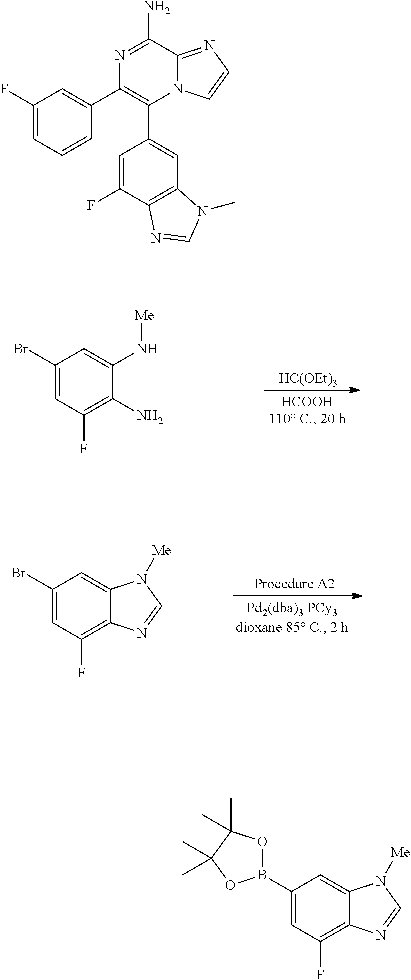 [SMILES: Cn1cnc2c(F)cc(-c3c(-c4cccc(F)c4)nc(N)c4nccn34)cc21 ; CNc1cc(Br)cc(F)c1N ; Cn1cnc2c(F)cc(Br)cc21 ; "HC(OEt)3" ; Cn1cnc2c(F)cc(B3OC(C)(C)C(C)(C)O3)cc21]

A) In a pressure tube were placed 2-amino-5-brom-3-fluoro-N-methylaniline (500 mg, 2.3 mmol) followed by triethylorthoformate (15 mL, 90 mmol) and formic acid (0.5 mL, 1.3 mmol). Reaction mixture was heated at 110° C. for 18 h. The reaction mixture was then cooled down to r.t., concentrated under reduced pressure to afford the crude material of 6-bromo-4-fluoro-1-methyl-1H-1,3-benzodiazole as an orange solid (264 mg, 50%). ESI-MS: 228.9 [M+H]+.

B) 4-Fluoro-1-methyl-6-(4,4,5,5-tetramethyl-1,3,2-dioxaborolan-2-yl)-1H-1,3-benzodiazole was prepared following the approach outlined in Procedure A2, substituting 6-bromo-4-fluoro-1-methyl-1H-1,3-benzodiazole for 5-bromo-1H-indazole and using 1,4-dioxane for DMF and substituting Pd₂(dba)₃ with tricyclohexyl phosphine for Pd(dppf)Cl₂ and heating for 2 h at 85° C. The reaction mixture was filtered through Celite® and the filtrate was concentrated under reduced pressure to give a brown oil (quant.) which was used in next step without further purification. ESI-MS: 277.2 [M+H]+.

C) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 4-fluoro-1-methyl-6-(4,4,5,5-tetramethyl-1,3,2-dioxaborolan-2-yl)-1H-1,3-benzodiazole for 5-(tetramethyl-1,3,2-dioxaborolan-2-yl)-1H-indazole in step (d) and using the conditions A described in Procedure D heating at 130° C. for 3 h to lead to the title compound (33 mg, 26%) as a hydrochloride salt as an off-white solid. ESI-MS: 377.1 [M+H]+. 1H NMR (400 MHz, DMSO-d6) δ 8.32 (s, 1H), 7.58 (d, J=1.3 Hz, 1H), 7.52 (d, J=1.2 Hz, 1H), 7.42 (d, J=1.2 Hz, 1H), 7.23-7.11 (m, 4H), 7.10-7.03 (m, 2H), 7.00 (d, J=2.7 Hz, 1H), 3.80 (s, 3H).

### Example 116: 6-(3-fluorophenyl)-5-(1,8-naphthyridin-4-yl)imidazo[1,2-a]pyrazin-8-amine

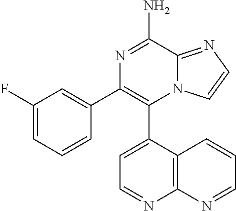 [SMILES: Nc1nc(-c2cccc(F)c2)c(-c2ccnc3ncccc23)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 1,8-naphthyridin-4-ylboronic acid for 5-(tetramethyl-1,3,2-dioxaborolan-2-yl)-1H-indazole in step (d) and using the conditions B described in Procedure D heating at 150° C. for 3 h to lead to the title compound (32 mg, 17%) as a hydrochloride salt as a brown solid. ESI-MS: 357.1 [M+H]+. 1H NMR (400 MHz, DMSO-d6) δ 9.26 (d, J=4.4 Hz, 1H), 9.19-9.16 (m, 1H), 8.49 (d, J=8.2 Hz, 1H), 7.90-7.85 (m, 2H), 7.67 (dd, J=8.4, 4.3 Hz, 1H), 7.54 (s, 1H), 7.25-7.09 (m, 3H), 6.99 (d, J=7.8 Hz, 1H).

### Example 117: ethyl 8-amino-6-(3-fluorophenyl)-5-(8-fluoroquinolin-6-yl)imidazo[1,2-a]pyrazine-2-carboxylate

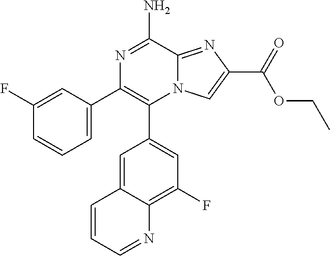 [SMILES: CCOC(=O)c1cn2c(-c3cc(F)c4ncccc4c3)c(-c3cccc(F)c3)nc(N)c2n1]

The title compound was synthesized following the approach outlined in Procedure B, Conditions B in step (c) substituting 3-fluorophenylboronic acid for phenylboronic acid in step (b) and substituting 8-fluoro-6-(tetramethyl-1,3,2-dioxaborolan-2-yl)quinoline for (3-chloro-4-hydroxyphenyl)-boronic acid in step (c) and substituting 3-bromopyruvic acid ethyl ester for chloroacetaldehyde in step (e) and performing this step in DME as a solvent heating at 80° C. for 16 h and substituting 0.5M NH₃ in dioxane for ammonium in water in step (f) to lead to the title compound (3 mg, 34%) as a light beige solid. ESI-MS: 446.10 [M+H]+. 1H NMR (400 MHz, Acetonitrile-d3) 9.02 (dd, J=4.2, 1.5 Hz, 1H), 8.30 (d, J=8.5 Hz, 1H), 7.92 (s, 1H), 7.81 (s, 1H), 7.62 (dd, J=8.4, 4.2 Hz, 1H), 7.48 (dd, J=11.2, 1.7 Hz, 1H), 7.28-7.02 (m, 2H), 7.05-6.83 (m, 1H), 6.22 (s, 1H), 4.32 (q, J=7.1 Hz, 2H), 1.31 (t, J=7.1 Hz, 3H).

### Example 118: [8-amino-6-(3-fluorophenyl)-5-(8-fluoroquinolin-6-yl)imidazo[1,2-a]pyrazin-2-yl]methanol

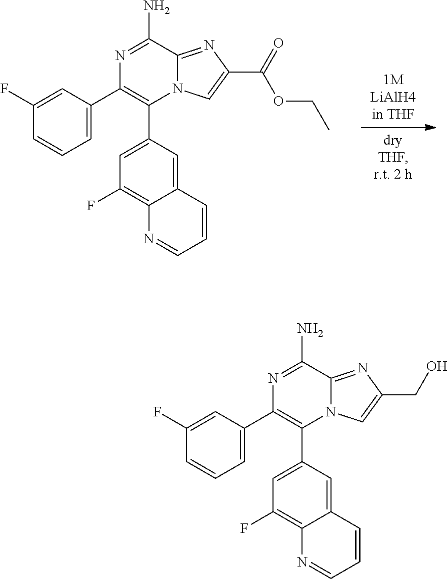 [SMILES: CCOC(=O)c1cn2c(-c3cc(F)c4ncccc4c3)c(-c3cccc(F)c3)nc(N)c2n1 ; Nc1nc(-c2cccc(F)c2)c(-c2cc(F)c3ncccc3c2)n2cc(CO)nc12]

In a dry flask was placed ethyl 8-amino-6-(3-fluorophenyl)-5-(8-fluoroquinolin-6-yl)imidazo[1,2-a]pyrazine-2-carboxylate (example 117) (10 mg, 0.02 mmol) and then THE (0.5 mL) was added and reaction mixture was cooled to 0° C. Then 1M LiAlH₄ in THE (0.07 mL, 0.07 mmol) was added and reaction was stirred at r.t. for 2 h. Then 10% NaOH (aq.) was added and aqueous phase was extracted with DCM. Organic layers were combined, dried over Na₂SO₄, filtered and concentrated to give a residue which was purified by using HPLC to lead to the title compound as a hydrochloride salt (2.5 mg, 29%) as a yellow solid. ESI-MS: 404.2 [M+H]+. 1H NMR (400 MHz, Methanol-d4) δ 9.03 (d, J=3.2 Hz, 1H), 8.45 (d, J=8.4 Hz, 1H), 7.96 (s, 1H), 7.74 (dd, J=8.4, 4.4 Hz, 1H), 7.69-7.56 (m, 2H), 7.43-7.07 (m, 4H), 4.77 (s, 2H).

### Example 119: 6-(3-fluorophenyl)-5-[2-methyl-6-(pyrrolidin-1-yl)pyridin-4-yl]imidazo[1,2-a]pyrazin-8-amine

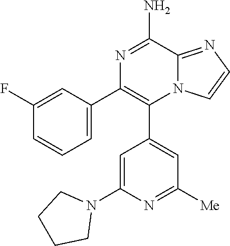 [SMILES: Cc1cc(-c2c(-c3cccc(F)c3)nc(N)c3nccn23)cc(N2CCCC2)n1]

In a pressure tube was placed 5-(2-fluoro-6-methylpyridin-4-yl)-6-(3-fluorophenyl)imidazo[1,2-a]pyrazin-8-amine (example 46) (50 mg, 0.1 mmol) and anhydrous dioxane (1 mL) was added followed by pyrrolidine (0.25 mL, 3.0 mmol). Reaction mixture was then heated at 80° C. for 20 h. After that time the reaction mixture was cooled down to r.t. then concentrated under reduced pressure and remaining residue was purified by flash chromatography on silica eluting with dichloromethane/methanol. Title product was obtained as a hydrochloride salt as a yellow solid (7 mg, 12%). ESI-MS: 389.2 [M+H]+. 1H NMR (400 MHz, DMSO-d6) δ 12.68 (s, 1H), 8.22 (s, 2H), 7.91 (s, 1H), 7.82 (s, 1H), 7.43-7.35 (m, 1H), 7.33-7.27 (m, 1H), 7.26-7.19 (m, 2H), 7.00 (s, 1H), 6.60 (s, 1H), 3.49 (s, 4H), 2.45 (s, 3H), 1.99 (s, 4H).

### Example 120:5-{8-fluoroimidazo[1,2-a]pyridin-6-yl}-6-(3-fluorophenyl)imidazo[1,2-a]pyrazin-8-amine

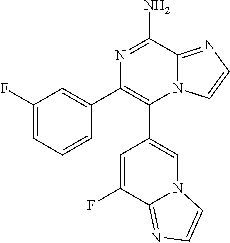 [SMILES: Nc1nc(-c2cccc(F)c2)c(-c2cc(F)c3nccn3c2)n2ccnc12]

A) {8-Fluoroimidazo[1,2-a]pyridin-6-yl}boronic acid was prepared following the approach outlined in Procedure A2, substituting 6-bromo-8-fluoroimidazo[1,2-a]pyridine for 5-bromo-1H-indazole and using 1,4-dioxane for DMF and heating for 3 h at 80° C. Product was obtained as a brown solid (quant.). ESI-MS: 181.1 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting {8-Fluoroimidazo[1,2-a]pyridin-6-yl}boronic acid for 5-(tetramethyl-1,3,2-dioxaborolan-2-yl)-1H-indazole in step (d) and using the conditions B described in Procedure D heating at 120° C. for 2 h to lead to the title compound (4 mg, 26%) as a hydrochloride salt as a beige solid. ESI-MS: 363.1 [M+H]+. 1H NMR (400 MHz, DMSO-d6) δ 8.55 (d, J=1.1 Hz, 1H), 8.11 (d, J=1.9 Hz, 1H), 7.80 (s, 1H), 7.74 (d, J=5.2 Hz, 2H), 7.41 (d, J=11.5 Hz, 1H), 7.36-7.22 (m, 2H), 7.14 (dd, J=16.0, 8.3 Hz, 2H).

### Example 121:2-[8-amino-5-(8-fluoroquinolin-6-yl)imidazo[1,2-a]pyrazin-6-yl]phenol

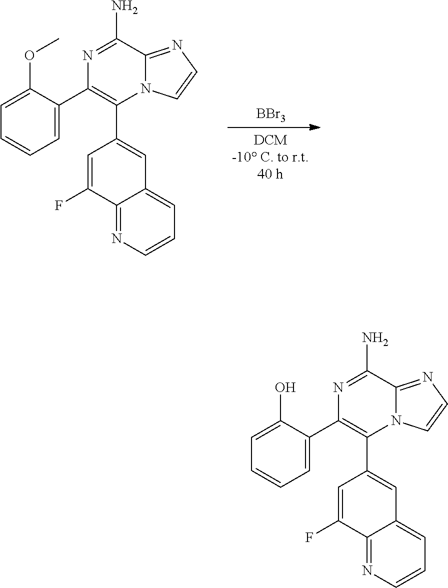 [SMILES: COc1ccccc1-c1nc(N)c2nccn2c1-c1cc(F)c2ncccc2c1 ; "BBr3" ; Nc1nc(-c2ccccc2O)c(-c2cc(F)c3ncccc3c2)n2ccnc12]

Step 1) 5-(8-Fluoroquinolin-6-yl)-6-(2-methoxyphenyl)imidazo[1,2-a]pyrazin-8-amine was synthesized following the approach outlined in Procedure B, Conditions B in step (c) and substituting 2-methoxyphenylboronic acid for phenyl boronic acid in step (b) and substituting 8-fluoro-6-(tetramethyl-1,3,2-dioxaborolan-2-yl)quinoline for (3-chloro-4-hydroxyphenyl)-boronic acid in step (c) to afford the product as an off-white solid (50 mg, 77%). ESI-MS: 386.00 [M+H]+. 1H NMR (300 MHz, DMSO-d6) δ 8.94 (dd, J=4.2, 1.4 Hz, 1H), 8.33 (d, J=8.4 Hz, 1H), 7.76 (s, 1H), 7.65-7.54 (m, 3H), 7.49 (dd, J=11.7, 1.3 Hz, 1H), 7.25 (dd, J=7.4, 1.6 Hz, 1H), 7.21-7.05 (m, 3H), 6.89-6.74 (m, 2H), 3.47 (s, 3H).

Step 2) In a flask was placed 5-(8-fluoroquinolin-6-yl)-6-(2-methoxyphenyl)imidazo[1,2-a]pyrazin-8-amine (29 mg, 0.08 mmol) then anhydrous DCM (5 mL) was added, mixture was cooled to −10° C. and then 1M solution of BBr₃ in DCM (0.23 mL, 0.23 mmol) was added. Reaction was then left to warm to r.t. and stirred for 40 h. After that time reaction mixture was poured onto ice and stirred for 20 minutes. Mixture was neutralized with NaHCO₃ and extracted with DCM.

Combined organics layers were washed with brine, dried over Na₂SO₄, filtered and concentrated. Crude product was purified by using HPLC (with formic acid) to lead to the title compound (18 mg, 64%) as a white solid. ESI-MS: 372.10 [M+H]+. 1H NMR (400 MHz, DMSO-d6) δ 9.89 (s, 1H), 8.97 (dd, J=4.2, 1.6 Hz, 1H), 8.36 (d, J=8.4 Hz, 1H), 7.86 (d, J=1.4 Hz, 1H), 7.63 (dd, J=8.4, 4.2 Hz, 1H), 7.61-7.54 (m, 3H), 7.26 (s, 2H), 7.03-6.97 (m, 2H), 6.68 (dd, J=8.6, 1.1 Hz, 1H), 6.60-6.54 (m, 1H).

### Example 122: 6-(6-fluoropyridin-2-yl)-5-(8-fluoroquinolin-6-yl)imidazo[1,2-a]pyrazin-8-amine

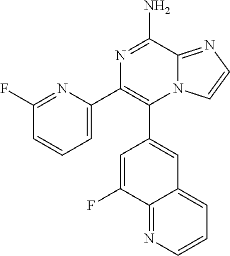 [SMILES: Nc1nc(-c2cccc(F)n2)c(-c2cc(F)c3ncccc3c2)n2ccnc12]

The title compound was synthesized following the approach outlined in Procedure B, Conditions B in step (c), substituting 3(6-fluoropirydyn-2-yl)boronic acid for phenylboronic acid in step (b) and performing this reaction in a mixture of solvents: toluene/ethanol 1:5, and substituting 8-fluoro-6-(tetramethyl-1,3,2-dioxaborolan-2-yl)quinoline for (3-chloro-4-hydroxyphenyl)-boronic acid in step (c) and substituting Pd(amphos)Cl₂ for Pd(dpp)Cl₂*DCM in step (c) and performing this step (f) or 3 h in 140° C. to lead to the title compound as a hydrochloride salt (28 mg, 37%) as a yellow solid. ESI-MS: 375.60 [M+H]+. 1H NMR (400 MHz, DMSO-d6) δ 9.07 (dd, J=4.3, 1.6 Hz, 1H), 8.68 (s, 2H), 8.51-8.46 (m, 1H), 7.97-7.93 (m, 1H), 7.93-7.86 (m, 1H), 7.85-7.81 (m, 1H), 7.77-7.69 (m, 3H), 7.35-7.26 (m, 1H), 7.15-7.10 (m, 1H).

### Example 123: 5-[8-amino-6-(3-fluorophenyl)imidazo[1,2-a]pyrazin-5-yl]-1-ethyl-1,2-dihydropyridin-2-one

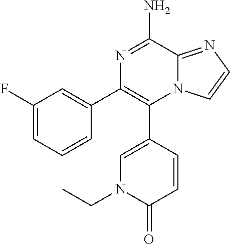 [SMILES: CCn1cc(-c2c(-c3cccc(F)c3)nc(N)c3nccn23)ccc1=O]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (1-ethyl-1,6-dihydro-6-oxo-3-pyridinyl)boronic acid for 5-(tetramethyl-1,3,2-dioxaborolan-2-yl)-1H-indazole in step (d) and using the conditions B described in Procedure B to lead to the title compound (9 mg, 9%) as a hydrochloride salt as a beige solid. ESI-MS: 350.1 [M+H]+. 1H NMR (400 MHz, DMSO-d6) δ 8.38 (s, 2H), 7.87 (s, 1H), 7.85 (s, 1H), 7.77 (d, J=2.4 Hz, 1H), 7.46-7.39 (m, 2H), 7.29-7.16 (m, 3H), 6.46 (d, J=9.4 Hz, 1H), 1.03 (t, J=7.1 Hz, 3H).

### Example 124: 6-(3-fluorophenyl)-5-{1H-pyrrolo[2,3-b]pyridin-3-yl}imidazo[1,2-a]pyrazin-8-amine

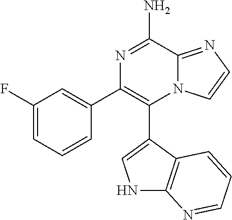 [SMILES: Nc1nc(-c2cccc(F)c2)c(-c2c[nH]c3ncccc23)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 3-(4,4,5,5-tetramethyl-1,3,2-dioxaborolan-2-yl)-1H-pyrrolo[2,3-b]pyridine for 5-(tetramethyl-1,3,2-dioxaborolan-2-yl)-1H-indazole in step (d) and using the conditions A described in Procedure D heating at 130° C. for 3 h to lead to the title compound (15 mg, 13%) as a hydrochloride salt as a yellow solid. ESI-MS: 345.1 [M+H]+. 1H NMR (400 MHz, DMSO-d6) δ 12.29 (s, 1H), 8.78 (s, 3H), 8.28 (dd, J=4.7, 1.4 Hz, 1H), 7.88 (s, 1H), 7.77 (d, J=2.7 Hz, 1H), 7.74-7.64 (m 2H), 7.35-7.21 (m 2H), 7.20-7.09 (m 2H), 7.05 (dd, J=7.9, 4.7 Hz, 1H).

### Example 125: 5-(5,8-difluoroquinolin-6-yl)-6-(3-fluorophenyl)imidazo[1,2-a]pyrazin-8-amine

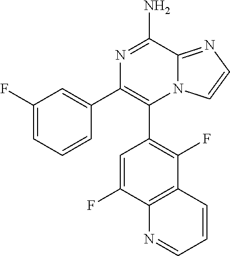 [SMILES: Nc1nc(-c2cccc(F)c2)c(-c2cc(F)c3ncccc3c2F)n2ccnc12]

A) (5,8-Difluoroquinolin-6-yl)boronic acid was prepared following the approach outlined in Procedure A2, substituting 6-bromo-5,8-difluoroquinoline for 5-bromo-1H-indazole and using 1,4-dioxane for DMF and heating for 18 h at 80° C. The reaction mixture was filtered through Celite® and the filtrate was concentrated under reduced pressure to give a white solid (quant.) which was used in next step without further purification. ESI-MS: 210.5 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (5,8-difluoroquinolin-6-yl)boronic acid for 5-(tetramethyl-1,3,2-dioxaborolan-2-yl)-1H-indazole in step (d) and using the conditions B described in Procedure D heating at 120° C. for 5 h to lead to the title compound (8 mg, 13%) as a hydrochloride salt as a beige solid. ESI-MS: 392.1 [M+H]+. 1H NMR (400 MHz, DMSO-d6) δ 9.13 (dd, J=4.2, 1.5 Hz, 1H), 8.56 (d, J=8.5 Hz, 1H), 8.35 (s, 2H), 7.87-7.76 (m, 3H), 7.72 (dd, J=10.7, 6.0 Hz, 1H), 7.36-7.22 (m, 2H), 7.19-7.04 (m, 2H).

### Example 126: 6-[8-amino-6-(3-fluorophenyl)imidazo[1,2-a]pyrazin-5-yl]quinolin-8-amine

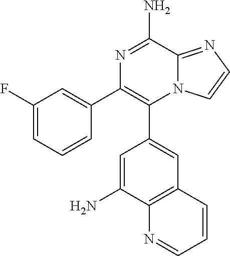 [SMILES: Nc1cc(-c2c(-c3cccc(F)c3)nc(N)c3nccn23)cc2cccnc12]

A) (8-Aminoquinolin-6-yl)boronic acid was prepared following the approach outlined in Procedure A2, substituting 6-bromo-8-quinolinamine for 5-bromo-1H-indazole and using 1,4-dioxane for DMF and heating for 2 h at 80° C. The reaction mixture was filtered through Celite® and the filtrate was concentrated under reduced pressure to give a red solid (quant.) which was used in next step without further purification. ESI-MS: 189.1 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (8-aminoquinolin-6-yl)boronic acid for 5-(tetramethyl-1,3,2-dioxaborolan-2-yl)-1H-indazole in step (d) and using the conditions B described in Procedure D heating at 120° C. for 2 h to lead to the title compound (35 mg, 28%) as a hydrochloride salt as an orange solid. ESI-MS: 371.2 [M+H]+. 1H NMR (300 MHz, DMSO-d6) δ 8.87 (dd, J=4.3, 1.6 Hz, 1H), 8.32 (dd, J=8.4, 1.7 Hz, 1H), 7.92 (d, J=1.3 Hz, 1H), 7.71 (d, J=1.4 Hz, 1H), 7.61 (dd, J=8.3, 4.3 Hz, 1H), 7.38-7.25 (m, 3H), 7.25-7.11 (m, 2H), 6.96 (d, J=1.8 Hz, 1H).

### Example 127: ethyl 2-[8-amino-6-(3-fluorophenyl)-5-(8-fluoroquinolin-6-yl)imidazo[1,2-a]pyrazin-2-yl]acetate

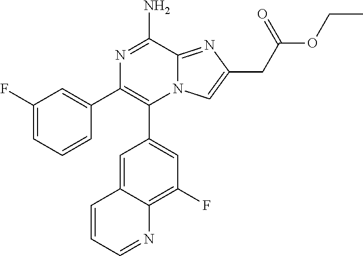 [SMILES: CCOC(=O)Cc1cn2c(-c3cc(F)c4ncccc4c3)c(-c3cccc(F)c3)nc(N)c2n1]

The title compound was synthesized following the approach outlined in Procedure B, Conditions B in step (c), substituting 3-fluorophenylboronic acid for phenylboronic acid in step (b) and substituting 8-fluoro-6-(tetramethyl-1,3,2-dioxaborolan-2-yl)quinoline for (3-chloro-4-hydroxyphenyl)-boronic acid in step (c) and substituting ethyl 4-chloroacetoacetate for chloroacetaldehyde in step (e) and performing this step in DME as a solvent heating at 85° C. for 16 h and substituting 0.5M NH₃ in dioxane for 28% ammonium in water in step (f) to lead to the title compound (12 mg, 22%) as a yellow solid. ESI-MS: 460.20 [M+H]+. 1H NMR (400 MHz, CDCl3) δ 9.07 (dd, J=4.2, 1.6 Hz, 1H), 8.15 (d, J=8.4 Hz, 1H), 7.63 (s, 1H), 7.60-7.51 (m, 1H), 7.42 (dd, J=10.6, 1.7 Hz, 1H), 7.37 (s, 1H), 7.20-7.06 (m, 2H), 7.07-7.00 (m, 1H), 6.96-6.84 (m, 1H), 5.66 (s, 2H), 4.20 (q, J=7.1 Hz, 2H), 3.83 (s, 2H), 1.28 (t, J=7.1 Hz, 3H).

### Example 128:5-(7-fluoro-1H-indazol-5-yl)-6-(3-fluorophenyl)imidazo[1,2-a]pyrazin-8-amine

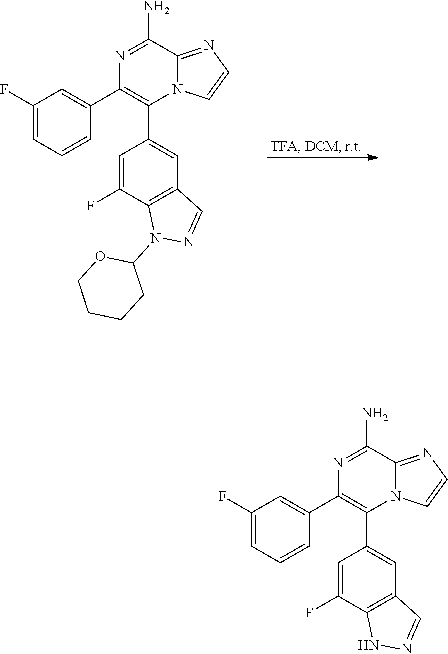 [SMILES: Nc1nc(-c2cccc(F)c2)c(-c2cc(F)c3c(cnn3C3CCCCO3)c2)n2ccnc12 ; "TFA, DCM, r.t." ; Nc1nc(-c2cccc(F)c2)c(-c2cc(F)c3[nH]ncc3c2)n2ccnc12]

A) 7-Fluoro-1-(tetrahydro-2H-pyran-2-yl)-5-(4,4,5,5-tetramethyl-1,3,2-dioxaborolan-2-yl)-1H-indazole was prepared following the approach outlined in Procedure A2, substituting 5-bromo-7-fluoro-1-(tetrahydro-2H-pyran-2-yl)-1H-indazole for 5-bromo-1H-indazole and substituting 1,4-dioxane for DMF and heating for 18 h at 100° C. The reaction mixture was filtered through Celite® and the filtrate was concentrated under reduced pressure to give a dark brown solid (quant.) which was used in next step without further purification. ESI-MS: 347.2 [M+H]+.

B) 5-[7-Fluoro-1-(oxan-2-yl)-1H-indazol-5-yl]-6-(3-fluorophenyl)imidazo[1,2-a]pyrazin-8-amine was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 7-fluoro-1-(tetrahydro-2H-pyran-2-yl)-5-(4,4,5,5-tetramethyl-1,3,2-dioxaborolan-2-yl)-1H-indazole for 5-(tetramethyl-1,3,2-dioxaborolan-2-yl)-1H-indazole in step (d) and using the conditions B described in Procedure D heating at 135° C. for 3 h. The reaction mixture was then cooled down to r.t., filtered through Celite® and rinsed with EtOAc. The organic solution was washed with water and brine and aqueous layer was extracted with EtOAc. Separated organic layer was dried over anhydrous sodium sulfate, filtered and concentrated under reduced pressure to afford the crude material as orange residue. The obtained crude material was purified by flash chromatography on silica eluting with hexane/ethyl acetate to afford 5-[7-fluoro-1-(oxan-2-yl)-1H-indazol-5-yl]-6-(3-fluorophenyl)imidazo[1,2-a]pyrazin-8-amine as an orange solid (85 mg, 58%). ESI-MS: 447.2 [M+H]+.

C) 5-[7-Fluoro-1-(oxan-2-yl)-1H-indazol-5-yl]-6-(3-fluorophenyl)imidazo[1,2-a]pyrazin-8-amine (85 mg, 0.18 mmol) was dissolved in anhydrous DCM (4 mL) and to this solution trifluoroacetic acid (0.28 mL, 3.8 mmol) was added. Reaction was stirred at r.t. for 2 d. After this time the reaction mixture was concentrated under reduced pressure with methanol three times. The obtained crude material was purified by flash chromatography on silica eluting with dichloromethane/methanol and then it was transformed to hydrochloride salt. The title compound 5-(7-fluoro-1H-indazol-5-yl)-6-(3-fluorophenyl)imidazo[1,2-a]pyrazin-8-amine was obtained as a white solid (16 mg, 23%). ESI-MS: 363.1 [M+H]+. 1H NMR (400 MHz, DMSO-d6) δ 8.50 (s, 1H), 8.25 (d, J=3.3 Hz, 1H), 7.83 (s, 1H), 7.68-7.66 (m, 1H), 7.66-7.63 (m, 1H), 7.36-7.28 (m, 2H), 7.26-7.21 (m, 1H), 7.17-7.11 (m, 2H).

### Example 129: 6-(3-fluorophenyl)-5-(4-methylquinolin-6-yl)imidazo[1,2-a]pyrazin-8-amine

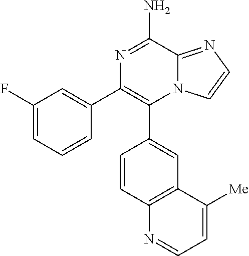 [SMILES: Cc1ccnc2ccc(-c3c(-c4cccc(F)c4)nc(N)c4nccn34)cc12]

A) (4-Methylquinolin-6-yl)boronic acid was prepared following the approach outlined in Procedure A2, substituting 6-bromo-4-methylquinoline for 5-bromo-1H-indazole and substituting 1,4-dioxane for DMF and heating for 20 h at 80° C. Product was obtained as a light brown oil (quant.) ESI-MS: 188.5 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (4-methylquinolin-6-yl)boronic acid for 5-(tetramethyl-1,3,2-dioxaborolan-2-yl)-1H-indazole in step (d) and using the conditions B described in Procedure D heating at 140° C. for 3 h to lead to the title compound (43 mg, 15%) as a hydrochloride salt as a yellow solid. ESI-MS: 370.2 [M+H]+. 1H NMR (400 MHz, DMSO-d6) δ 9.14 (d, J=5.2 Hz, 1H), 8.97 (s, 2H), 8.55 (d, J=1.3 Hz, 1H), 8.40 (d, J=8.8 Hz, 1H), 8.00-7.91 (m, 2H), 7.89 (d, J=5.3 Hz, 1H), 7.86 (d, J=1.3 Hz, 1H), 7.35-7.24 (m, 2H), 7.20-7.11 (m, 2H), 2.81 (s, 3H).

### Example 130: 6-[8-amino-6-(3-fluorophenyl)imidazo[1,2-a]pyrazin-5-yl]quinoline-8-carbonitrile

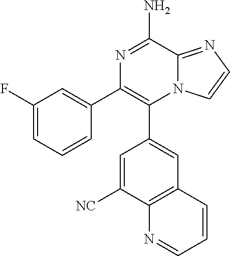 [SMILES: N#Cc1cc(-c2c(-c3cccc(F)c3)nc(N)c3nccn23)cc2cccnc12]

A) (8-Cyanoquinolin-6-yl)boronic acid was prepared following the approach outlined in Procedure A2, substituting 6-bromoquinoline-8-carbonitrile for 5-bromo-1H-indazole and using 1,4-dioxane for DMF and heating for 18 h at 100° C. The reaction mixture was filtered through Celite® and the filtrate was concentrated under reduced pressure to give a white dark brown (quant.) which was used in next step without further purification. ESI-MS: 199.1 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (8-cyanoquinolin-6-yl)boronic acid for 5-(tetramethyl-1,3,2-dioxaborolan-2-yl)-1H-indazole in step (d) and using the conditions B described in Procedure D heating at 140° C. for 3 h to lead to the title compound (13 mg, 3%) as a hydrochloride salt as a yellow solid. ESI-MS: 381.2 [M+H]+. 1H NMR (400 MHz, DMSO-d6) δ 9.16 (dd, J=4.3, 1.7 Hz, 1H), 8.53 (dd, J=8.4, 1.7 Hz, 1H), 8.44-8.41 (m, 2H), 8.22 (s, 1H), 7.82-7.75 (m, 3H), 7.30-7.22 (m, 2H), 7.15-7.05 (m, 2H).

### Example 131:5-{8-fluoro-[1,2,4]triazolo[1,5-a]pyridin-6-yl}-6-(3-fluorophenyl)imidazo[1,2-a]pyrazin-8-amine

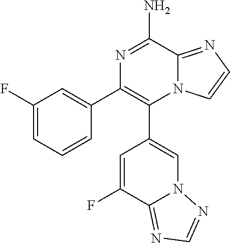 [SMILES: Nc1nc(-c2cccc(F)c2)c(-c2cc(F)c3ncnn3c2)n2ccnc12]

A) Preparation of {8-fluoro-[1,2,4]triazolo[1,5-a]pyridin-6-yl}boronic acid: {8-fluoro-[1,2,4]triazolo[1,5-a]pyridin-6-yl}boronic acid was prepared following the approach outlined in Procedure A2, substituting 6-bromo-8-fluoro-[1,2,4]triazolo[1,5-a]pyridine for 5-bromo-1H-indazole and using 1,4-dioxane for DMF and heating for 20 h at 80° C. The reaction mixture was filtered through Celite® and the filtrate was concentrated under reduced pressure to give a brown solid (quant.) which was used in next step without further purification. ESI-MS: 182.1 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting {8-fluoro-[1,2,4]triazolo[1,5-a]pyridin-6-yl}boronic acid for 5-(tetramethyl-1,3,2-dioxaborolan-2-yl)-1H-indazole in step (d) and using the conditions B described in Procedure B heating at 100° C. for 1 h to lead to the title compound (21 mg, 24%) as a hydrochloride salt as a yellow solid. ESI-MS: 364.1 [M+H]+. 1H NMR (300 MHz, DMSO-d6) δ 9.05 (d, J=1.3 Hz, 1H), 8.68 (s, 1H), 8.32 (s, 2H), 7.90 (s, 1H), 7.86-7.76 (m, 2H), 7.40-7.25 (m, 2H), 7.22-7.09 (m, 2H).

### Example 132: 5-(4-fluoro-1H-1,3-benzodiazol-6-yl)-6-(3-fluorophenyl)imidazo[1,2-a]pyrazin-8-amine

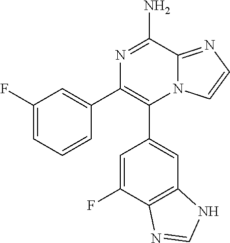 [SMILES: Nc1nc(-c2cccc(F)c2)c(-c2cc(F)c3nc[nH]c3c2)n2ccnc12]

A) 4-Fluoro-6-(4,4,5,5-tetramethyl-1,3,2-dioxaborolan-2-yl)-1H-1,3-benzodiazole was prepared following the approach outlined in Procedure A2, substituting 5-bromo-7-fluoro-1H-benzimidazole for 5-bromo-1H-indazole and using 1,4-dioxane for DMF and heating for 18 h at 80° C. The reaction mixture was filtered through Celite® and the filtrate was concentrated under reduced pressure to give a green solid (quant.) which was used in next step without further purification. ESI-MS: 263.1 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 4-fluoro-6-(4,4,5,5-tetramethyl-1,3,2-dioxaborolan-2-yl)-1H-1,3-benzodiazole for 5-(tetramethyl-1,3,2-dioxaborolan-2-yl)-1H-indazole in step (d) and using the conditions B described in Procedure D heating at 140° C. for 3 h to lead to the title compound (3 mg, 14%) as a hydrochloride salt as an off-white solid. ESI-MS: 363.1 [M+H]+. 1H NMR (400 MHz, DMSO-d6) δ 8.77 (s, 2H), 8.55 (s, 1H), 7.86 (d, J=1.3 Hz, 1H), 7.67 (d, J=1.3 Hz, 1H), 7.50 (d, J=1.3 Hz, 1H), 7.38-7.29 (m, 1H), 7.28-7.20 (m, 2H), 7.19-7.09 (m, 2H).

### Example 133: 4-[8-amino-6-(3-fluorophenyl)imidazo[1,2-a]pyrazin-5-yl]-2-fluoro-6-(trifluoromethyl)phenol

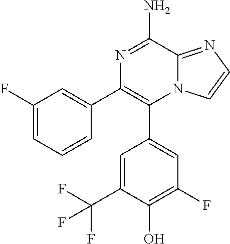 [SMILES: Nc1nc(-c2cccc(F)c2)c(-c2cc(F)c(O)c(C(F)(F)F)c2)n2ccnc12]

A) [3-fluoro-4-hydroxy-5-(trifluoromethyl)phenyl]boronic acid was prepared following the approach outlined in Procedure A2, substituting 4-Bromo-2-fluoro-6-(trifluoromethyl)phenol for 5-bromo-1H-indazole and using 1,4-dioxane for DMF and heating for 18 h at 80° C. The reaction mixture was filtered through Celite® and the filtrate was concentrated under reduced pressure to give a white solid (quant.) which was used in next step without further purification. ESI-MS: 223.1 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting [3-fluoro-4-hydroxy-5-(trifluoromethyl)phenyl]boronic acid for 5-(tetramethyl-1,3,2-dioxaborolan-2-yl)-1H-indazole in step (d) and using the conditions B described in Procedure D heating at 120° C. for 5 h to lead to the title compound (8 mg, 13%) as a hydrochloride salt as a white solid. ESI-MS: 407.1 [M+H]+. 1H NMR (400 MHz, DMSO-d6) δ 11.47 (s, 1H), 8.44 (s, 2H), 7.83 (s, 1H), 7.77 (s, 1H), 7.66 (dd, J=11.1, 1.9 Hz, 1H), 7.45-7.30 (m, 2H), 7.27-7.15 (m, 2H), 7.12 (d, J=7.8 Hz, 1H).

### Example 134: 6-[8-amino-6-(3-fluorophenyl)imidazo[1,2-a]pyrazin-5-yl]isoquinolin-1-ol

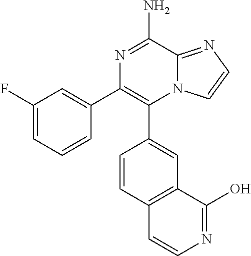 [SMILES: Nc1nc(-c2cccc(F)c2)c(-c2ccc3ccnc(O)c3c2)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (1,2-dihydro-1-oxo-6-isoquinolinyl)boronic acid for 5-(tetramethyl-1,3,2-dioxaborolan-2-yl)-1H-indazole in step (d) and using the conditions A described in Procedure D heating at 120° C. for 20 h to lead to the title compound (38 mg, 27%) as a hydrochloride salt as an orange solid. ESI-MS: 373.1 [M+H]+. 1H NMR (400 MHz, DMSO-d6) δ 11.43 (s, 1H), 8.23 (d, J=8.2 Hz, 1H), 7.88 (s, 1H), 7.75 (s, 1H), 7.68 (s, 1H), 7.47 (d, J=8.3 Hz, 1H), 7.32 (s, 1H), 7.25-7.10 (m, 4H), 6.53 (d, J=7.1 Hz, 1H).

### Example 135: 2-[8-amino-6-(3-fluorophenyl)-5-(8-fluoroquinolin-6-yl)imidazo[1,2-a]pyrazin-2-yl]acetic Acid

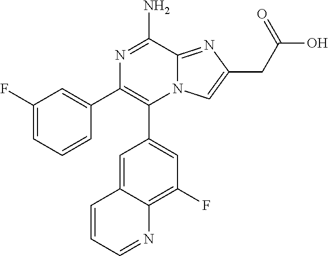 [SMILES: Nc1nc(-c2cccc(F)c2)c(-c2cc(F)c3ncccc3c2)n2cc(CC(=O)O)nc12]

The title compound was synthesized following the approach outlined in Procedure B, Conditions B in step (c), substituting 3-fluorophenylboronic acid for phenylboronic acid in step (b) and substituting 8-fluoro-6-(tetramethyl-1,3,2-dioxaborolan-2-yl)quinoline for (3-chloro-4-hydroxyphenyl)-boronic acid in step (c) and substituting ethyl 4-chloroacetoacetate for chloroacetaldehyde in step (e) and performing this step in DME as a solvent heating at 85° C. for 16 h to lead to the title compound (20 mg, 27%) as a yellow solid. ESI-MS: 432.20 [M+H]+. 1H NMR (400 MHz, DMSO-d6) δ 9.02 (dd, J=4.2, 1.6 Hz, 1H), 8.42 (d, J=8.5 Hz, 1H), 7.89 (s, 1H), 7.75-7.61 (m, 2H), 7.37 (s, 1H), 7.24-7.10 (m, 4H), 7.06 (d, J=7.9 Hz, 1H), 7.04-6.94 (m, 1H), 3.57 (s, 2H).

### Example 136: 5-[8-amino-6-(3-fluorophenyl)imidazo[1,2-a]pyrazin-5-yl]-2,3-dihydro-1H-isoindol-1-one

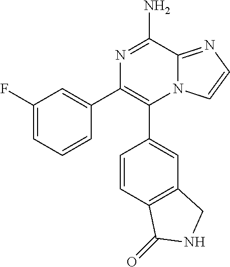 [SMILES: Nc1nc(-c2cccc(F)c2)c(-c2ccc3c(c2)CNC3=O)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 5-(4,4,5,5-tetramethyl-1,3,2-dioxaborolan-2-yl)isoindolin-1-one for 5-(tetramethyl-1,3,2-dioxaborolan-2-yl)-1H-indazole in step (d) and using the conditions A described in Procedure D heating at 120° C. for 20 h to lead to the title compound (32 mg, 23%) as a hydrochloride salt as an orange solid. ESI-MS: 360.2 [M+H]+. 1H NMR (400 MHz, DMSO-d6) δ 8.94 (s, 2H), 8.75 (s, 1H), 7.92 (s, 1H), 7.75 (d, J=7.8 Hz, 1H), 7.67 (d, J=14.0 Hz, 2H), 7.48 (d, J=7.7 Hz, 1H), 7.38-7.32 (m, 1H), 7.25-7.17 (m, 2H), 7.13 (d, J=7.7 Hz, 1H), 4.39 (s, 2H).

### Example 137: 5-[8-amino-6-(3-fluorophenyl)imidazo[1,2-a]pyrazin-5-yl]-2,3-dihydro-1H-inden-1-one

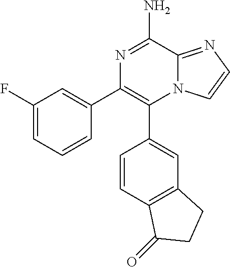 [SMILES: Nc1nc(-c2cccc(F)c2)c(-c2ccc3c(c2)CCC3=O)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 5-(4,4,5,5-tetramethyl-1,3,2-dioxaborolan-2-yl)-2,3-dihydro-1H-inden-1-one for 5-(tetramethyl-1,3,2-dioxaborolan-2-yl)-1H-indazole in step (d) and using the conditions A described in Procedure D heating at 120° C. for 2 days to lead to the title compound (38 mg, 27%) as a hydrochloride salt as an orange solid. ESI-MS: 360.1 [M+H]+. 1H NMR (400 MHz, DMSO-d6) δ 8.86 (s, 2H), 7.91 (s, 1H), 7.74-7.65 (m, 3H), 7.42 (d, J=8.0 Hz, 1H), 7.38-7.33 (m, 1H), 7.26-7.19 (m, 2H), 7.14 (d, J=7.8 Hz, 1H), 3.11 (s, 2H), 2.69 (s, 2H).

### Example 138: 2-[8-amino-6-(3-fluorophenyl)-5-(8-fluoroquinolin-6-yl)imidazo[1,2-a]pyrazin-2-yl]ethan-1-ol

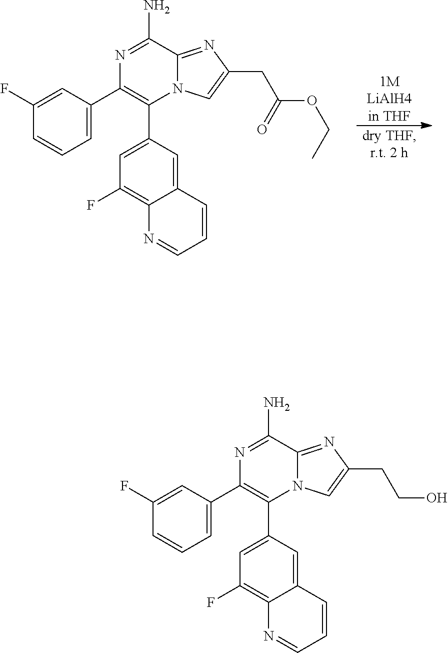 [SMILES: CCOC(=O)Cc1cn2c(-c3cc(F)c4ncccc4c3)c(-c3cccc(F)c3)nc(N)c2n1 ; "1M LiAlH4in THF" ; "dry THF,r.t. 2 h" ; Nc1nc(-c2cccc(F)c2)c(-c2cc(F)c3ncccc3c2)n2cc(CCO)nc12]

In a dry flask was placed ethyl 2-[8-amino-6-(3-fluorophenyl)-5-(8-fluoroquinolin-6-yl)imidazo[1,2-a]pyrazin-2-yl]acetate (example 127) (35 mg, 0.08 mmol) and then THE (1 mL) was added and reaction mixture was cooled to 0° C. Then 1M LiAlH₄ in THE (0.23 mL, 0.23 mmol) was added and reaction was stirred at r.t. for 0.5 h. Then 10% NaOH (aq.) was added and aqueous phase was extracted with DCM. Organic layers were combined, dried over Na₂SO₄, filtered and concentrated to give a crude mixture which was purified by using HPLC to lead to the title compound (5 mg, 14%) as a light yellow solid, hydrochloride salt. ESI-MS: 418.20 [M+H]⁺. ¹H NMR (400 MHz, DMSO-d6) 9.03 (dd, J=4.2, 1.7 Hz, 1H), 8.44 (dt, J=8.4, 1.6 Hz, 1H), 7.91 (s. 2H), 7.90 (d, J=1.6 Hz, 1H), 7.77-7.62 (m, 2H), 7.48 (s, 1H), 7.34-7.17 (m, 2H), 7.17-7.01 (m, 2H), 3.71 (t, J=6.8 Hz, 2H), 2.85 (t, J=6.7 Hz, 2H).

### Example 139: 2-[8-amino-6-(3-fluorophenyl)-5-(8-fluoroquinolin-6-yl)imidazo[1,2-a]pyrazin-2-yl]acetamide

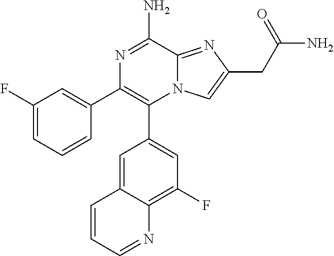 [SMILES: NC(=O)Cc1cn2c(-c3cc(F)c4ncccc4c3)c(-c3cccc(F)c3)nc(N)c2n1]

In a pressure tube ethyl 2-[8-amino-6-(3-fluorophenyl)-5-(8-fluoroquinolin-6-yl)imidazo[1,2-a]pyrazin-2-yl]acetate (example 127) (35 mg, 0.08 mmol) was suspended in 7N NH₃ in methanol (4 mL). Reaction was heated at 110° C. for 16 h then mixture was concentrated and purified by HPLC to lead to the title compound (5 mg, 13%) as light beige solid. ESI-MS: 431.20 [M+H]+. 1H NMR (300 MHz, DMSO-d6) δ 9.02 (dd, J=4.2, 1.5 Hz, 1H), 8.42 (d, J=8.5 Hz, 1H), 7.89 (s, 1H), 7.76-7.62 (m, 2H), 7.37 (s, 2H), 7.28-7.11 (m, 4H), 7.12-6.85 (m, 3H), 3.50 (s, 2H).

### Example 140:6-(3-fluorophenyl)-5-(4-methoxy-1,3-benzothiazol-6-yl)imidazo[1,2-a]pyrazin-8-amine

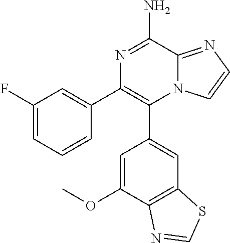 [SMILES: COc1cc(-c2c(-c3cccc(F)c3)nc(N)c3nccn23)cc2scnc12]

A) (4-Methoxy-1,3-benzothiazol-6-yl)boronic acid was prepared following the approach outlined in Procedure A2, substituting 6-bromo-4-methoxy-1,3-benzothiazole for 5-bromo-1H-indazole and using 1,4-dioxane for DMF and heating under microwave irradiation for 2 h at 120° C. The reaction mixture was filtered through Celite® and the filtrate was concentrated under reduced pressure to give a brown solid (quant.) which was used in next step without further purification. ESI-MS: 210.1 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (4-methoxy-1,3-benzothiazol-6-yl)boronic acid for 5-(tetramethyl-1,3,2-dioxaborolan-2-yl)-1H-indazole in step (d) and using the conditions B described in Procedure D heating under microwave irradiation at 130° C. for 30 min to lead to the title compound (133 mg, 62%) as a hydrochloride salt as a white solid. ESI-MS: 392.9 [M+H]+. 1H NMR (400 MHz, DMSO-d6) δ 9.37 (s, 1H), 9.00 (s, 2H), 7.93 (d, J=1.4 Hz, 1H), 7.80 (d, J=1.4 Hz, 1H), 7.77 (d, J=1.4 Hz, 1H), 7.34 (td, J=8.1, 6.1 Hz, 1H), 7.27 (dt, J=9.8, 2.2 Hz, 1H), 7.21-7.14 (m, 4H), 3.86 (s, 3H).

### Example 141: 6-[8-amino-6-(3-fluorophenyl)imidazo[1,2-a]pyrazin-5-yl]naphthalen-1-ol

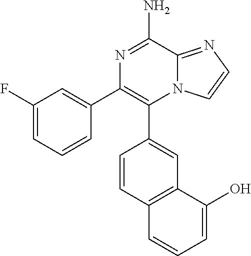 [SMILES: Nc1nc(-c2cccc(F)c2)c(-c2ccc3cccc(O)c3c2)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (5-hydroxynaphthalen-2-yl)boronic acid for 5-(tetramethyl-1,3,2-dioxaborolan-2-yl)-1H-indazole in step (d) and using the conditions B described in Procedure D heating at 135° C. for 3 h to lead to the title compound (26 mg, 15%) as a hydrochloride salt as a yellow solid. ESI-MS: 371.2 [M+H]+. 1H NMR (400 MHz, DMSO-d6) δ 10.35 (s, 1H), 8.54 (s, 1H), 8.19 (d, J=8.6 Hz, 1H), 7.90 (d, J=1.6 Hz, 1H), 7.82 (d, J=1.3 Hz, 1H), 7.60 (d, J=1.3 Hz, 1H), 7.42 (dd, J=8.7, 1.8 Hz, 1H), 7.39-7.32 (m, 2H), 7.32-7.25 (m, 1H), 7.25-7.20 (m, 1H), 7.17-7.13 (m, 1H), 7.11 (dd, J=8.7, 2.6 Hz, 1H), 6.97 (dd, J=7.1, 1.5 Hz, 1H).

### Example 142: 5-[4-fluoro-1-(propan-2-yl)-1H-1,3-benzodiazol-6-yl]-6-(3-fluorophenyl)imidazo[1,2-a]pyrazin-8-amine

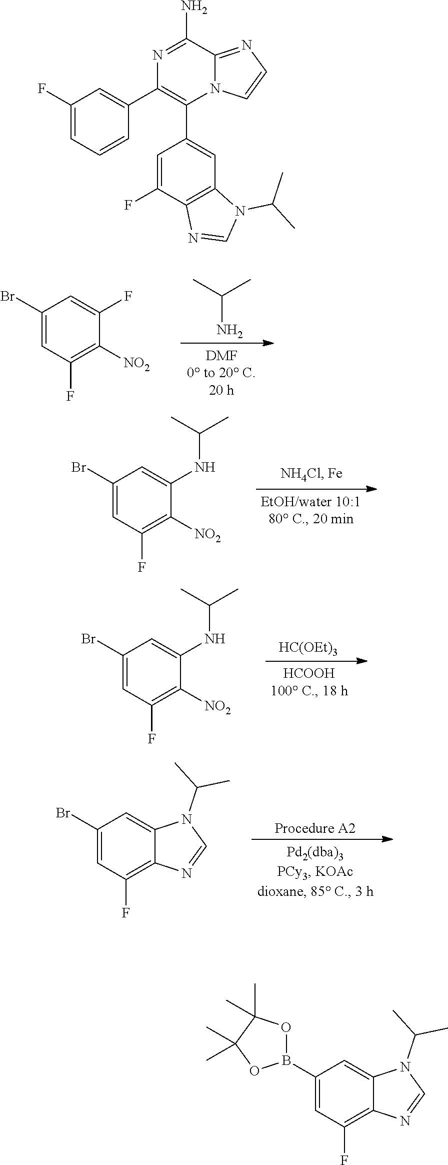

A) In a flask, equipped with stirring bar and a rubber septum, thoroughly purged with argon, 5-bromo-1,3-difluoro-2-nitrobenzene (700 mg, 2.9 mmol) was dissolved in DMF (30 mL) reaction was cooled to 0° C. and to this isopropylamine (0.13 mL, 1.47 mmol) was added at 0° C. After 30 min flask was left to warm to room temperature for 20 h. Reaction mixture was pured to brine and extracted with ethyl acetate twice. Separated and combined organic layers were dried over anhydrous sodium sulfate, filtered and concentrated under reduced pressure to afford the crude residue which was purified by flash chromatography on silica eluting with hexane/dichloromethane to lead to the 5-bromo-3-fluoro-2-nitro-N-(propan-2-yl)aniline as an orange oil (744 mg, 91%). ESI-MS: 276.0 [M+H]+.

B) In a flask equipped with condenser was placed 5-bromo-3-fluoro-2-nitro-N-(propan-2-yl)aniline (588 mg, 2.1 mmol), ammonium chloride (341 mg, 6.4 mmol) and iron powder (593 mg, 10.6 mmol). Solids were suspended in 10:1 ethanol/water mixture (22 mL) and reaction was heated at 80° C. for 20 min. The reaction mixture was filtered through Celite® and the filtrate was concentrated under reduced pressure and then purified by flash chromatography on silica eluting with hexane and dichloromethane to lead to 5-bromo-3-fluoro-1-N-(propan-2-yl)benzene-1,2-diamine as a purple solid (452 mg, 77%). ESI-MS: 347.5 [M+H]+.

C) In a pressure tube were placed 5-bromo-3-fluoro-1-N-(propan-2-yl)benzene-1,2-diamine (452 mg, 0.1.8 mmol) followed by triethylorthoformate (3.6 mL, 22 mmol) and formic acid (17 mg, 0.36 mmol). Reaction mixture was heated at 110° C. for 18 h. The reaction mixture was then cooled down to r.t., concentrated under reduced pressure to afford the crude material which was purified by flash chromatography on silica eluting with hexane and ethyl acetate to lead to 6-bromo-4-fluoro-1-(propan-2-yl)-1H-1,3-benzodiazole as a beige solid (365 mg, 78%).

D) 4-fluoro-1-(propan-2-yl)-6-(4,4,5,5-tetramethyl-1,3,2-dioxaborolan-2-yl)-1H-1,3-benzodiazole was prepared following the approach outlined in Procedure A2, substituting 6-bromo-4-fluoro-1-(propan-2-yl)-1H-1,3-benzodiazole for 5-bromo-1H-indazole and using 1,4-dioxane for DMF and substituting Pd₂(dba)₃ and tricyclohexylphosphine for Pd(dppf)Cl₂ and heating for 3 h at 85° C. The reaction mixture was filtered through Celite® and the filtrate was concentrated under reduced pressure to give a dark orange solid (quant.) which was used in next step without further purification. ESI-MS: 305.2 [M+H]+.

E) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 4-fluoro-1-(propan-2-yl)-6-(4,4,5,5-tetramethyl-1,3,2-dioxaborolan-2-yl)-1H-1,3-benzodiazole for 5-(tetramethyl-1,3,2-dioxaborolan-2-yl)-1H-indazole in step (d) and using the conditions B described in Procedure D heating at 135° C. for 3 h to lead to the title compound (74 mg, 67%) as a hydrochloride salt as a beige solid. ESI-MS: 405.2 [M+H]+. 1H NMR (400 MHz, DMSO-d6) δ 8.82 (s, 1H), 8.56 (s, 1H), 7.89 (d, J=1.4 Hz, 1H), 7.78 (d, J=1.4 Hz, 1H), 7.60 (d, J=1.3 Hz, 1H), 7.37-7.28 (m, 1H), 7.29-7.23 (m, 1H), 7.23-7.19 (m, 1H), 7.19-7.10 (m, 2H), 4.73-4.64 (m, 1H), 1.46 (s, 3H), 1.40 (s, 3H).

### Example 143: 6-(3-fluorophenyl)-5-(3-methyl-1H-indazol-5-yl)imidazo[1,2-a]pyrazin-8-amine

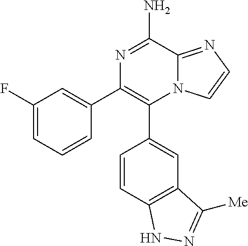 [SMILES: Cc1n[nH]c2ccc(-c3c(-c4cccc(F)c4)nc(N)c4nccn34)cc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (3-methyl-1H-indazol-5-yl)boronic acid for 5-(tetramethyl-1,3,2-dioxaborolan-2-yl)-1H-indazole in step (d) and using the conditions A described in Procedure D heating at 120° C. for 20 h to lead to the title compound (13 mg, 27%) as a hydrochloride salt as a white solid. ESI-MS: 359.1 [M+H]+. 1H NMR (400 MHz, DMSO-d6) δ 8.82 (s, 2H), 7.88 (s, 1H), 7.84 (s, 1H), 7.62 (d, J=1.3 Hz, 1H), 7.54 (dd, J=8.6, 0.8 Hz, 1H), 7.34-7.28 (m, 2H), 7.25-7.20 (m, 1H), 7.19-7.14 (m, 2H), 2.45 (s, 3H).

### Example 144:5-[8-amino-6-(3-fluorophenyl)imidazo[1,2-a]pyrazin-5-yl]-1-ethyl-3-fluoro-1,2-dihydropyridin-2-one

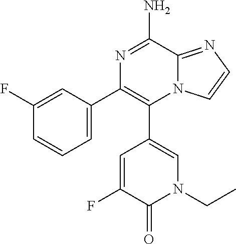 [SMILES: CCn1cc(-c2c(-c3cccc(F)c3)nc(N)c3nccn23)cc(F)c1=O]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (in step (d) and using the conditions A described in Procedure D heating at 120° C. for 20 h to lead to the title compound (26 mg, 25%) as a hydrochloride salt as a beige solid. ESI-MS: 368.2 [M+H]+. 1H NMR (400 MHz, DMSO-d6) δ 7.98 (s, 1H), 7.88 (s, 1H), 7.65 (dd, J=2.3, 1.1 Hz, 1H), 7.53 (dd, J=10.6, 2.2 Hz, 1H), 7.48-7.41 (m, 1H), 7.31-7.18 (m, 3H), 1.04 (t, J=7.1 Hz, 3H).

### Example 145: 6-[8-amino-6-(3-fluorophenyl)imidazo[1,2-a]pyrazin-5-yl]quinolin-3-amine

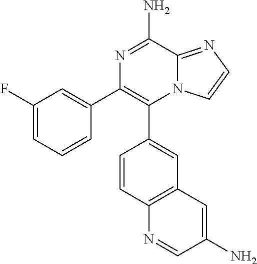 [SMILES: Nc1cnc2ccc(-c3c(-c4cccc(F)c4)nc(N)c4nccn34)cc2c1]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (3-aminoquinolin-6-yl)boronic acid for 5-(tetramethyl-1,3,2-dioxaborolan-2-yl)-1H-indazole in step (d) and using the conditions B described in Procedure D except substituting Pd Sphos G3 for Pd(amphos)Cl₂ and heating under microwave irradiation at 130° C. for 20 h to lead to the title compound (46 mg, 29%) as a hydrochloride salt as a yellow solid. ESI-MS: 371.2 [M+H]+. 1H NMR (400 MHz, DMSO-d6) δ 9.32 (s, 1H), 8.84 (s, 2H), 8.08-7.87 (m, 3H), 7.49-7.36 (m, 1H), 7.33-7.24 (m, 2H), 7.24-7.08 (m, 2H), 6.73 (dd, J=6.6, 1.4 Hz, 1H), 2.97 (s, 3H).

### Example 146: 5-(4-fluoro-1,3-benzothiazol-6-yl)-6-(4-fluorophenyl)imidazo[1,2-a]pyrazin-8-amine

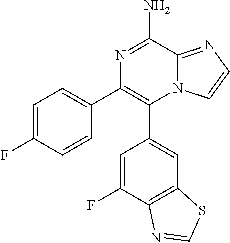 [SMILES: Nc1nc(-c2ccc(F)cc2)c(-c2cc(F)c3ncsc3c2)n2ccnc12]

A) (4-Fluoro-1,3-benzothiazol-6-yl)boronic acid was prepared following the approach outlined in Procedure A2, substituting 6-bromo-4-fluoro-1,3-benzothiazole for 5-bromo-1H-indazole and using 1,4-dioxane for DMF and heating for 2 h at 80° C. Product was obtained as a light yellow solid (0.671 g, quant.). ESI-MS: 198.10 [M+H]+.

B) The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (4-fluoro-1,3-benzothiazol-6-yl)boronic acid for 5-(tetramethyl-1,3,2-dioxaborolan-2-yl)-1H-indazole in step (d) and using the conditions B described in Procedure D heating at 130° C. for 3 h to lead to the title compound (97 mg, 67%) as a hydrochloride salt as an off-white solid. ESI-MS: 370.2 [M+H]+. 1H NMR (400 MHz, DMSO-d6) δ 8.94 (d, J=3.8 Hz, 1H), 8.33-8.29 (m, 1H), 8.14 (d, J=8.2 Hz, 1H), 7.78 (d, J=7.4 Hz, 2H), 7.75-7.71 (m, 1H), 7.63-7.57 (m, 1H), 7.43-7.36 (m, 2H), 7.11 (t, J=8.7 Hz, 2H), 2.66 (s, 3H).

### Example 147:3-[8-amino-5-(4-fluoro-1,3-benzothiazol-6-yl)imidazo[1,2-a]pyrazin-6-yl]benzonitrile

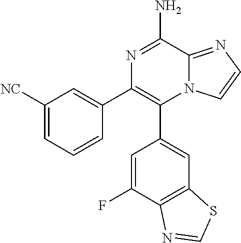 [SMILES: N#Cc1cccc(-c2nc(N)c3nccn3c2-c2cc(F)c3ncsc3c2)c1]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (4-fluoro-1,3-benzothiazol-6-yl)boronic acid (example 146) for 5-(tetramethyl-1,3,2-dioxaborolan-2-yl)-1H-indazole in step (d) and using the conditions B described in Procedure D heating at 100° C. for 1 h to lead to the title compound (30 mg, 65%) as a hydrochloride salt as a yellow solid. ESI-MS: 387.1 [M+H]+. 1H NMR (400 MHz, DMSO-d6) δ 9.54 (s, 1H), 8.26 (s, 3H), 8.10 (d, J=1.4 Hz, 1H), 7.91-7.83 (m, 2H), 7.80-7.70 (m, 2H), 7.60-7.53 (m, 2H), 7.49-7.43 (m, 1H).

### Example 148: 6-(4-fluorophenyl)-5-(4-methylquinolin-6-yl)imidazo[1,2-a]pyrazin-8-amine

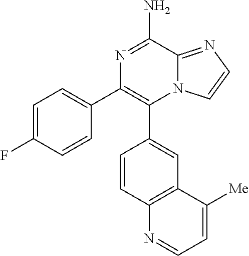 [SMILES: Cc1ccnc2ccc(-c3c(-c4ccc(F)cc4)nc(N)c4nccn34)cc12]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (4-methylquinolin-6-yl)boronic acid (example 129) for 5-(tetramethyl-1,3,2-dioxaborolan-2-yl)-1H-indazole in step (d) and using the conditions B described in Procedure D heating at 140° C. for 3 h to lead to the title compound (90 mg, 46%) as a hydrochloride salt as a yellow solid. ESI-MS: 370.2 [M+H]+. 1H NMR (400 MHz, DMSO-d6) δ 9.05 (d, J=5.0 Hz, 1H), 8.43 (s, 1H), 8.24 (d, J=8.7 Hz, 1H), 7.95-7.71 (m, 4H), 7.42 (dd, J=8.5, 5.4 Hz, 2H), 7.14 (t, J=8.7 Hz, 2H), 2.73 (s, 3H).

### Example 149: 5-(1,3-benzothiazol-6-yl)-6-(4-fluorophenyl)imidazo[1,2-a]pyrazin-8-amine

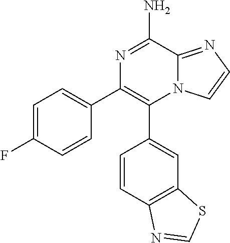 [SMILES: Nc1nc(-c2ccc(F)cc2)c(-c2ccc3ncsc3c2)n2ccnc12]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (1,3-benzothiazol-6-yl)boronic acid for 5-(tetramethyl-1,3,2-dioxaborolan-2-yl)-1H-indazole in step (d) and using the conditions B described in Procedure D heating at 130° C. for 3 h to lead to the title compound (45 mg, 62%) as a hydrochloride salt as an off-white solid. ESI-MS: 362.2 [M+H]+. 1H NMR (400 MHz, DMSO-d6) δ 9.52 (s, 1H), 8.96 (s, 1H), 8.30 (d, J=1.6 Hz, 1H), 8.17 (d, J=8.4 Hz, 1H), 7.89 (d, J=1.4 Hz, 1H), 7.68 (d, J=1.3 Hz, 1H), 7.54 (dd, J=8.4, 1.8 Hz, 1H), 7.44-7.37 (m, 2H), 7.21-7.13 (m, 2H).

### Example 150: 6-(3-fluorophenyl)-5-(quinazolin-6-yl)imidazo[1,2-a]pyrazin-8-amine

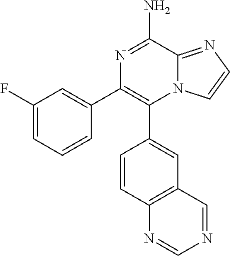 [SMILES: Nc1nc(-c2cccc(F)c2)c(-c2ccc3ncncc3c2)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 6-methoxy-3-pyridinylboronic acid for 5-(tetramethyl-1,3,2-dioxaborolan-2-yl)-1H-indazole in step (d) and using the conditions A described in Procedure D heating at 115° C. for 20 h to lead to the title compound (33 mg, 40%) as a beige solid. ESI-MS: 357.2 [M+H]+. 1H NMR (400 MHz, DMSO-d6) δ 9.60 (s, 1H), 9.36 (s, 1H), 8.31 (s, 1H), 8.09-7.92 (m, 2H), 7.55 (d, J=13.4 Hz, 2H), 7.28 (s, 2H), 7.19-7.13 (m, 2H), 7.02 (d, J=8.4 Hz, 2H).

### Example 151: 5-(8-chloroquinolin-6-yl)-6-(4-fluorophenyl)imidazo[1,2-a]pyrazin-8-amine

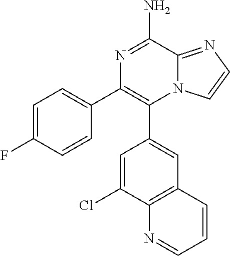 [SMILES: Nc1nc(-c2ccc(F)cc2)c(-c2cc(Cl)c3ncccc3c2)n2ccnc12]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (8-chloroquinolin-6-yl)boronic acid for 5-(tetramethyl-1,3,2-dioxaborolan-2-yl)-1H-indazole in step (d) and using the conditions B described in Procedure D heating at 130° C. for 3 h to lead to the title compound (49 mg, 43%) as a hydrochloride salt as a yellow solid. ESI-MS: 390.2 [M+H]+. 1H NMR (400 MHz, DMSO-d6) δ 9.11 (dd, J=4.2, 1.7 Hz, 1H), 8.48 (dd, J=8.4, 1.7 Hz, 1H), 8.11 (d, J=1.8 Hz, 1H), 7.96 (d, J=1.8 Hz, 1H), 7.95-7.91 (m, 1H), 7.85 (d, J=1.3 Hz, 1H), 7.72 (dd, J=8.3, 4.2 Hz, 1H), 7.47-7.41 (m, 2H), 7.22-7.15 (m, 2H).

### Example 152:5-(8-fluoro-4-methylquinolin-6-yl)-6-(4-fluorophenyl)imidazo[1,2-a]pyrazin-8-amine

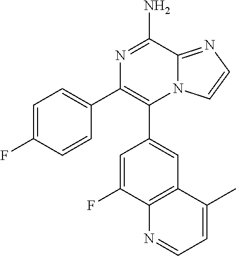 [SMILES: Cc1ccnc2c(F)cc(-c3c(-c4ccc(F)cc4)nc(N)c4nccn34)cc12]

A) (8-Fluoro-4-methylquinolin-6-yl)boronic acid was prepared following the approach outlined in Procedure A2, substituting 6-bromo-8-fluoro-4-methylquinoline for 5-bromo-1H-indazole and using 1,4-dioxane for DMF and heating for 20 h at 100° C. The reaction mixture was filtered through Celite® and the filtrate was concentrated under reduced pressure to give a yellow solid (quant.) which was used in next step without further purification. ESI-MS: 206.15 [M+H]+.

B) The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (8-Fluoro-4-methylquinolin-6-yl)boronic acid for 5-(tetramethyl-1,3,2-dioxaborolan-2-yl)-1H-indazole in step (d) and using the conditions A described in Procedure D except substituting Pd(PPh₃)₄ for Pd(dppf)Cl₂ to lead to the title compound (8 mg, 50%) as a hydrochloride salt as a yellow solid. ESI-MS: 388.2 [M+H]+. 1H NMR (400 MHz, DMSO-d6) δ 8.88 (d, J=4.4 Hz, 1H), 8.02 (d, J=1.6 Hz, 1H), 7.92 (s, 1H), 7.90 (d, J=1.2 Hz, 1H), 7.64 (dd, J=11.0, 1.7 Hz, 1H), 7.56 (dd, J=4.4, 1.1 Hz, 1H), 7.50-7.41 (m, 2H), 7.18 (t, J=8.9 Hz, 2H), 2.57 (d, J=0.9 Hz, 3H).

### Example 153: 6-(4-fluorophenyl)-5-(1-methyl-1H-1,3-benzodiazol-6-yl)imidazo[1,2-a]pyrazin-8-amine

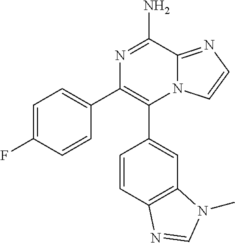 [SMILES: Cn1cnc2ccc(-c3c(-c4ccc(F)cc4)nc(N)c4nccn34)cc21]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (1-methyl-1H-benzimidazol-6-yl)boronic acid for 5-(tetramethyl-1,3,2-dioxaborolan-2-yl)-1H-indazole in step (d) and using the conditions B described in Procedure D heating at 130° C. for 3 h to lead to the title compound (33 mg, 43%) as a hydrochloride salt as an off-white solid. ESI-MS: 359.2 [M+H]+. 1H NMR (400 MHz, DMSO-d6) δ 9.50 (s, 1H), 9.07 (s, 2H), 8.15 (s, 1H), 7.96-7.87 (m, 2H), 7.67 (d, J=1.3 Hz, 1H), 7.48-7.38 (m, 3H), 7.20-7.11 (m, 2H), 4.02 (s, 3H).

### Example 154:5-(4-fluoro-1-methyl-1H-1,3-benzodiazol-6-yl)-6-(4-fluorophenyl)imidazo[1,2-a]pyrazin-8-amine

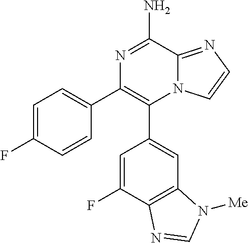 [SMILES: Cn1cnc2c(F)cc(-c3c(-c4ccc(F)cc4)nc(N)c4nccn34)cc21]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting 4-fluoro-1-methyl-6-(4,4,5,5-tetramethyl-1,3,2-dioxaborolan-2-yl)-1H-1,3-benzodiazole (example 115) for 5-(tetramethyl-1,3,2-dioxaborolan-2-yl)-1H-indazole in step (d) and using the conditions B described in Procedure D heating at 135° C. for 3 h to lead to the title compound (37 mg, 38%) as a hydrochloride salt as an off-white solid. ESI-MS: 377.2 [M+H]+. 1H NMR (400 MHz, DMSO-d6) δ 8.42 (s, 1H), 7.88 (d, J=1.3 Hz, 1H), 7.72 (d, J=1.3 Hz, 1H), 7.60 (d, J=1.3 Hz, 1H), 7.47-7.40 (m, 2H), 7.22-7.15 (m, 2H), 7.10 (dd, J=11.2, 1.3 Hz, 1H), 3.82 (s, 3H).

### Example 155: 6-(3-fluorophenyl)-5-{3-methylimidazo[1,2-a]pyridin-6-yl}imidazo[1,2-a]pyrazin-8-amine

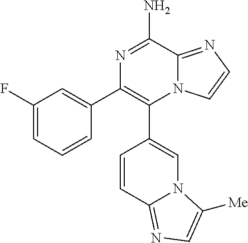 [SMILES: Cc1cnc2ccc(-c3c(-c4cccc(F)c4)nc(N)c4nccn34)cn12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 3-methyl-6-(4,4,5,5-tetramethyl-1,3,2-dioxaborolan-2-yl)imidazo[1,2-a]pyridine for 5-(tetramethyl-1,3,2-dioxaborolan-2-yl)-1H-indazole in step (d) and using the conditions A described in Procedure D heating at 130° C. for 1 h to lead to the title compound (19 mg, 30%) as a hydrochloride salt as a white solid. ESI-MS: 359.2 [M+H]+. 1H NMR (400 MHz, DMSO-d6) δ 9.13 (t, J=1.3 Hz, 1H), 8.49 (s, 2H), 8.09 (d, J=1.3 Hz, 1H), 8.01 (dd, J=9.3, 0.9 Hz, 1H), 7.96 (d, J=1.7 Hz, 1H), 7.89 (s, 1H), 7.74 (dd, J=9.3, 1.5 Hz, 1H), 7.37-7.27 (m, 2H), 7.23-7.12 (m, 2H), 2.53 (d, J=1.1 Hz, 3H).

### Example 156: 3-(8-amino-5-{8-methylimidazo[1,2-a]pyridin-6-yl}imidazo[1,2-a]pyrazin-6-yl)benzonitrile

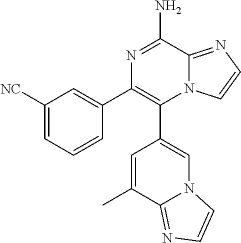 [SMILES: Cc1cc(-c2c(-c3cccc(C#N)c3)nc(N)c3nccn23)cn2ccnc12]

A) {8-methylimidazo[1,2-a]pyridin-6-yl}boronic acid was prepared following the approach outlined in Procedure A2, substituting 6-bromo-8-methylimidazo[1,2-a]pyridine for 5-bromo-1H-indazole and using 1,4-dioxane for DMF and heating for 20 h at 80° C. The reaction mixture was filtered through Celite® and the filtrate was concentrated under reduced pressure to give a beige solid (quant.) which was used in next step without further purification. ESI-MS: 177.2 [M+H]+.

B) The title compound was synthesized following the approach outlined in Procedure D, substituting 6-chloro-5-(3-cyanophenyl)pyrazin-2-amine [Procedure B, step (b) substituting 3-cyanophenylboronic acid for phenylboronic acid] for 6-chloro-5-phenylpyrazin-2-amine in step (a) and substituting {8-methylimidazo[1,2-a]pyridin-6-yl}boronic acid for 5-(tetramethyl-1,3,2-dioxaborolan-2-yl)-1H-indazole in step (d) and using the conditions B described in Procedure D heating at 100° C. for 1 h to lead to the title compound (8 mg, 0.02 mmol, 9%) as a hydrochloride salt as a yellow solid. ESI-MS: 366.2 [M+H]+. 1H NMR (400 MHz, DMSO-d6) δ 8.89 (s, 1H), 8.34 (d, J=2.1 Hz, 1H), 8.27 (d, J=2.1 Hz, 1H), 7.93 (s, 1H), 7.81-7.69 (m, 4H), 7.62-7.56 (m, 1H), 7.48-7.40 (m, 1H), 2.59 (s, 3H).

### Example 157: 3-[8-amino-5-(8-chloroquinolin-6-yl)imidazo[1,2-a]pyrazin-6-yl]benzonitrile

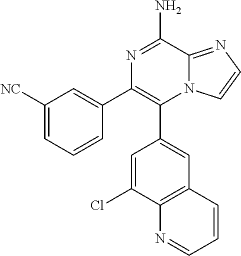 [SMILES: N#Cc1cccc(-c2nc(N)c3nccn3c2-c2cc(Cl)c3ncccc3c2)c1]

The title compound was synthesized following the approach outlined in Procedure D, substituting 6-chloro-5-(3-cyanophenyl)pyrazin-2-amine [Procedure B, step (b) substituting 3-cyanophenylboronic acid for phenylboronic acid] for 6-chloro-5-phenylpyrazin-2-amine in step (a) and substituting (8-chloroquinolin-6-yl)boronic acid for 5-(tetramethyl-1,3,2-dioxaborolan-2-yl)-1H-indazole in step (d) and using the conditions B described in Procedure D heating at 90° C. for 1 h to lead to the title compound (13 mg, 27%) as a hydrochloride salt as a yellow solid. ESI-MS: 397.2 [M+H]+. 1H NMR (400 MHz, DMSO-d6) δ 9.10 (dd, J=4.2, 1.7 Hz, 1H), 8.47 (dd, J=8.4, 1.7 Hz, 1H), 8.11 (d, J=1.8 Hz, 1H), 8.00 (d, J=1.8 Hz, 1H), 7.92-7.88 (m, 1H), 7.85 (d, J=1.4 Hz, 1H), 7.81-7.69 (m, 3H), 7.61-7.55 (m, 1H), 7.48-7.41 (m, 1H).

### Example 158: 3-[8-amino-5-(1,3-benzothiazol-6-yl)imidazo[1,2-a]pyrazin-6-yl]benzonitrile

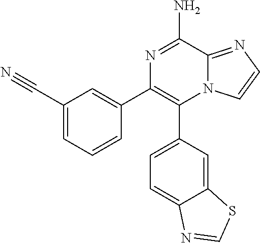 [SMILES: N#Cc1cccc(-c2nc(N)c3nccn3c2-c2ccc3ncsc3c2)c1]

The title compound was synthesized following the approach outlined in Procedure B, Conditions B in step (c), substituting 3-cyanophenylboronic pinacolate ester for phenylboronic acid in step (b) and performing the reaction for 8 h in 80° C., and substituting 6-(tetramethyl-1,3,2-dioxaborolan-2-yl)-1,3-benzothiazole for (3-chloro-4-hydroxyphenyl)-boronic acid in step (c) and performing this step (f) or 2 h in 140° C., to lead to the title compound (3 mg, 14%) as a pale yellow solid. ESI-MS: 369.9 [M+H]+. 1H NMR (400 MHz, DMSO-d6) δ 9.49 (s, 1H), 8.29 (d, J=1.6 Hz, 1H), 8.17 (d, J=8.4 Hz, 1H), 7.77 (s, 1H), 7.65-7.62 (m, 1H), 7.58-7.54 (m, 2H), 7.52-7.47 (m, 1H), 7.43 (d, J=1.2 Hz, 1H), 7.39-7.32 (m, 1H), 7.25 (s, 2H).

### Example 159: 6-(3-fluorophenyl)-5-[5-(1H-pyrazol-5-yl)thiophen-2-yl]imidazo[1,2-a]pyrazin-8-amine

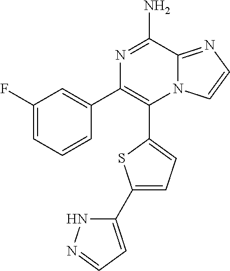 [SMILES: Nc1nc(-c2cccc(F)c2)c(-c2ccc(-c3ccn[nH]3)s2)n2ccnc12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting [5-(1H-pyrazol-5-yl)thiophen-2-yl]boronic acid for 5-(tetramethyl-1,3,2-dioxaborolan-2-yl)-1H-indazole in step (d) and using the conditions A described in Procedure D heating at 130° C. for 1.5 h to lead to the title compound (14 mg, 20%) as a hydrochloride salt as a beige solid. ESI-MS: 377.2 [M+H]+. 1H NMR (400 MHz, DMSO-d6) δ 8.85 (s, 2H), 7.93 (d, J=1.4 Hz, 1H), 7.88 (d, J=1.4 Hz, 1H), 7.79 (d, J=2.3 Hz, 1H), 7.47-7.38 (m, 2H), 7.34-7.27 (m, 3H), 7.23 (td, J=7.6, 6.6, 1.6 Hz, 1H), 6.67 (d, J=2.3 Hz, 1H).

### Example 160: 3-[8-amino-5-(4-methylquinolin-6-yl)imidazo[1,2-a]pyrazin-6-yl]benzonitrile

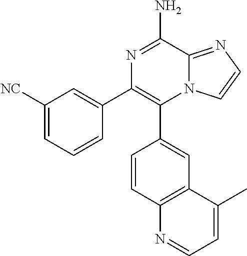 [SMILES: Cc1ccnc2ccc(-c3c(-c4cccc(C#N)c4)nc(N)c4nccn34)cc12]

The title compound was synthesized following the approach outlined in Procedure D, substituting 6-chloro-5-(3-cyanophenyl)pyrazin-2-amine [Procedure B, step (b) substituting 3-cyanophenylboronic acid for phenylboronic acid] for 6-chloro-5-phenylpyrazin-2-amine in step (a) and substituting (4-methylquinolin-6-yl)boronic acid (example 129) for 5-(tetramethyl-1,3,2-dioxaborolan-2-yl)-1H-indazole in step (d) and using the conditions B described in Procedure D to lead to the title compound (14 mg, 57%) as a hydrochloride salt as a yellow solid. ESI-MS: 377.3 [M+H]+. 1H NMR (400 MHz, DMSO-d6) δ 9.07 (d, J=5.1 Hz, 1H), 8.46 (s, 1H), 8.27 (d, J=8.7 Hz, 1H), 7.94-7.88 (m, 2H), 7.85 (s, 1H), 7.81-7.71 (m, 3H), 7.59-7.55 (m, 1H), 7.45-7.39 (m, 1H), 2.75 (s, 3H).

### Example 161: 3-[8-amino-5-(8-methoxyquinolin-6-yl)imidazo[1,2-a]pyrazin-6-yl]benzonitrile

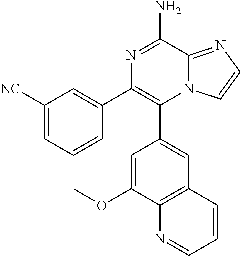 [SMILES: COc1cc(-c2c(-c3cccc(C#N)c3)nc(N)c3nccn23)cc2cccnc12]

A) (8-Methoxyquinolin-6-yl)boronic acid was prepared following the approach outlined in Procedure A2, substituting 6-bromo-5-methoxyquinoline for 5-bromo-1H-indazole and using 1,4-dioxane for DMF and heating for 20 h at 120° C. The reaction mixture was filtered through Celite® and the filtrate was concentrated under reduced pressure to give a dark brown solid (quant.) which was used in next step without further purification. ESI-MS: 204.05 [M+H]+.

B) The title compound was synthesized following the approach outlined in Procedure D, substituting 6-chloro-5-(3-cyanophenyl)pyrazin-2-amine [Procedure B, step (b) substituting 3-cyanophenylboronic acid for phenylboronic acid] for 6-chloro-5-phenylpyrazin-2-amine in step (a) and substituting (8-methoxyquinolin-6-yl)boronic acid for 5-(tetramethyl-1,3,2-dioxaborolan-2-yl)-1H-indazole in step (d) and using the conditions B described in Procedure D to lead to the title compound (4 mg, 0.01 mmol, 4%) as a hydrochloride salt as a yellow solid. ESI-MS: 393.3 [M+H]+. 1H NMR (400 MHz, DMSO-d6) δ 8.96 (dd, J=4.4, 1.6 Hz, 1H), 8.44 (d, J=8.3 Hz, 1H), 7.91 (s, 1H), 7.81-7.65 (m, 4H), 7.64 (d, J=1.6 Hz, 1H), 7.61-7.56 (m, 1H), 7.46-7.37 (m, 1H), 7.32 (s, 1H), 3.89 (s, 3H).

### Example 162:3-[8-amino-5-(1-methyl-1H-1,3-benzodiazol-6-yl)imidazo[1,2-a]pyrazin-6-yl]benzonitrile

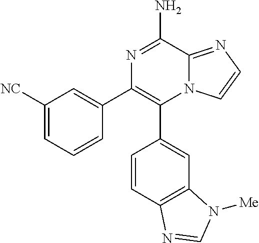 [SMILES: Cn1cnc2ccc(-c3c(-c4cccc(C#N)c4)nc(N)c4nccn34)cc21]

The title compound was synthesized following the approach outlined in Procedure D, substituting 6-chloro-5-(3-cyanophenyl)pyrazin-2-amine [Procedure B, step (b) substituting 3-cyanophenylboronic acid for phenylboronic acid] for 6-chloro-5-phenylpyrazin-2-amine in step (a) and substituting (1-methyl-1H-benzimidazol-6-yl)boronic acid for 5-(tetramethyl-1,3,2-dioxaborolan-2-yl)-1H-indazole in step (d) and using the conditions B described in Procedure D to lead to the title compound (6 mg, 7%) as a hydrochloride salt as a yellow solid. ESI-MS: 366.2 [M+H]+. 1H NMR (400 MHz, DMSO-d6) δ 9.29 (s, 1H), 8.10 (s, 1H), 7.91-7.81 (m, 2H), 7.78 (d, J=1.3 Hz, 1H), 7.73 (ddd, J=7.8, 1.4 Hz, 1H), 7.58-7.52 (m, 2H), 7.47-7.37 (m, 2H), 3.98 (s, 3H).

### Example 163:6-[8-amino-6-(3-fluorophenyl)imidazo[1,2-a]pyrazin-5-yl]-N-methylquinolin-8-amine

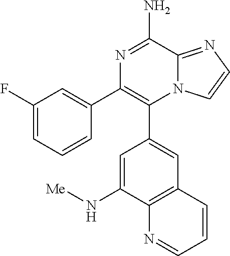 [SMILES: CNc1cc(-c2c(-c3cccc(F)c3)nc(N)c3nccn23)cc2cccnc12]

In a dry pressure tube was placed 6-(3-fluorophenyl)-5-(8-fluoroquinolin-6-yl)imidazo[1,2-a]pyrazin-8-amine (example 61) (50 mg, 0.12 mmol) then it was dissolved in NMP (1 mL) and then 2M methylamine in THE (0.6 mL, 1.2 mmol) was added. Reaction was then heated at 180° C. for 1 d. After cooling to room temperature, the crude reaction mixture was concentrated under reduced pressure. The resulting residue was purified by column chromatography, eluting with hexane/ethyl acetate and then additional purification by RP-HPLC (formic acid) was performed. The title product was obtained as a hydrochloride salt as an orange solid (4 mg, 13%). ESI-MS: 385.9 [M+H]+. 1H NMR (400 MHz, DMSO-d6) δ 8.79 (dd, J=4.2, 1.7 Hz, 1H), 8.20 (dd, J=8.4, 1.7 Hz, 1H), 7.80 (s, 1H), 7.71 (s, 1H), 7.54 (dd, J=8.3, 4.2 Hz, 1H), 7.34-7.20 (m, 3H), 7.17-7.08 (m, 2H), 6.53 (d, J=1.7 Hz, 1H), 2.77 (s, 3H).

### Example 164: 6-[8-amino-6-(3-fluorophenyl)imidazo[1,2-a]pyrazin-5-yl]-N,N-dimethylquinolin-8-amine

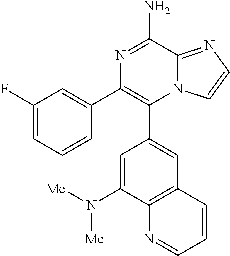 [SMILES: CN(C)c1cc(-c2c(-c3cccc(F)c3)nc(N)c3nccn23)cc2cccnc12]

In a dry pressure tube was placed 6-(3-fluorophenyl)-5-(8-fluoroquinolin-6-yl)imidazo[1,2-a]pyrazin-8-amine (example 61) (50 mg, 0.12 mmol) then it was dissolved in NMP (1 mL) and then 2M dimethylamine in THE (0.6 mL, 1.2 mmol) was added. Reaction was then heated at 180° C. for 1 d. After cooling to room temperature, the crude reaction mixture was concentrated under reduced pressure. The resulting residue was purified by column chromatography, eluting with hexane/ethyl acetate and then additional purification by RP-HPLC (formic acid) was performed. The title product was obtained as a hydrochloride salt as a yellow solid (9 mg, 38%). ESI-MS: 399.2 [M+H]+. 1H NMR (400 MHz, DMSO-d6) δ 8.86 (dd, J=4.1, 1.8 Hz, 1H), 8.25 (dd, J=8.4, 1.8 Hz, 1H), 7.60 (d, J=1.2 Hz, 1H), 7.57-7.47 (m, 3H), 7.25-7.07 (m, 5H), 7.05-6.95 (m, 1H), 6.88 (d, J=1.8 Hz, 1H), 2.93 (s, 6H).

### Example 165:5-(4-chloro-1,3-benzothiazol-6-yl)-6-(4-fluorophenyl)imidazo[1,2-a]pyrazin-8-amine

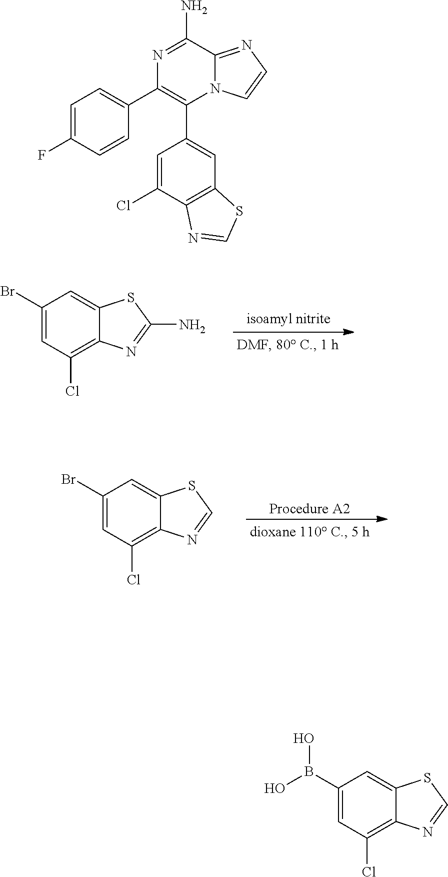 [SMILES: Nc1nc(-c2ccc(F)cc2)c(-c2cc(Cl)c3ncsc3c2)n2ccnc12 ; Nc1nc2c(Cl)cc(Br)cc2s1 ; Clc1cc(Br)cc2scnc12 ; "Procedure A2" ; OB(O)c1cc(Cl)c2ncsc2c1]

A) In a flask 6-bromo-4-chloro-1,3-benzothiazol-2-amine (298 mg, 1.2 mmol) was dissolved in DMF (5 mL) and then isoamyl nitrite (0.49 mL, 3.6 mmol) was added dropwise. Reaction was heated at 80° C. for 1 h. After cooling to room temperature, the crude reaction mixture was concentrated under reduced pressure. The resulting residue was purified by column chromatography, eluting with hexane/ethyl acetate mixture to afford the 6-bromo-4-chloro-1,3-benzothiazole as an off-white solid (111 mg, 62%). ESI-MS: 249.9 [M+H]+.

B) (4-Chloro-1,3-benzothiazol-6-yl)boronic acid was prepared following the approach outlined in Procedure A2, substituting 6-bromo-4-chloro-1,3-benzothiazole for 5-bromo-1H-indazole and using 1,4-dioxane for DMF and heating for 5 h at 110° C. The reaction mixture was filtered through Celite® and the filtrate was concentrated under reduced pressure to give a brown oil (quant.) which was used in next step without further purification. ESI-MS: 213.1 [M+H]+.

C) The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (4-chloro-1,3-benzothiazol-6-yl)boronic acid for 5-(tetramethyl-1,3,2-dioxaborolan-2-yl)-1H-indazole in step (d) and using the conditions B described in Procedure D heating at 100° C. for 1 h to lead to the title compound (14 mg, 17%) as a hydrochloride salt as an off-white solid. ESI-MS: 396.2 [M+H]+. 1H NMR (400 MHz, DMSO-d6) δ 9.58 (s, 1H), 8.26 (d, J=1.6 Hz, 1H), 7.75 (s, 1H), 7.69 (d, J=1.5 Hz, 1H), 7.66 (s, 1H), 7.42-7.36 (m, 2H), 7.18-7.11 (m, 2H).

### Example 166:8-amino-6-(3-cyanophenyl)-5-(quinolin-6-yl)imidazo[1,2-a]pyrazine-2-carboxamide

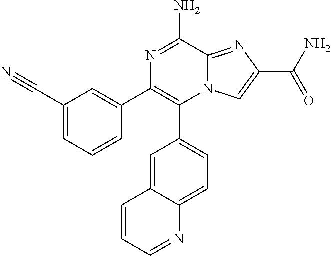 [SMILES: N#Cc1cccc(-c2nc(N)c3nc(C(N)=O)cn3c2-c2ccc3ncccc3c2)c1]

The title compound was synthesized following the approach outlined in Procedure B, Conditions A in step (c), substituting 3-cyanophenylboronic acid for phenylboronic acid in step (b) and substituting (quinolin-6-yl)boronic acid for (3-chloro-4-hydroxyphenyl)-boronic acid in step (c) and substituting Pd(amphos)Cl₂ for Pd(PPh₃)₄ in step (c) and performing this step for 3 h at 140° C. and substituting 3-bromopyruvic acid ethyl ester for chloroacetaldehyde in step (e) and performing this step in DME as a solvent heating at 85° C. for 48 h and substituting 0.5M NH₃ in dioxane for NH₃ in water in step (f) and performing this reaction for 20 h at 100° C. then heating with 7N NH₃ in methanol at 100° C. for 6 h to lead to the title compound (3 mg, 12%) as an orange solid. ESI-MS: 406.30 [M+H]⁺. ¹H NMR (400 MHz, DMSO-d6) 8.99 (dd, J=4.2, 1.7 Hz, 1H), 8.37 (dd, J=8.5, 1.9 Hz, 1H), 8.18-8.06 (m, 2H), 7.81 (d, J=1.4 Hz, 2H), 7.77 (dd, J=8.7, 2.0 Hz, 1H), 7.65 (dt, J=7.7, 1.4 Hz, 1H), 7.60 (dd, J=8.3, 4.2 Hz, 1H), 7.54 (dd, J=3.0, 1.7 Hz, 1H), 7.52 (t, J=1.5 Hz, 1H), 7.47 (s, 1H), 7.40 (s, 2H), 7.35 (t, J=7.8 Hz, 1H).

### Example 167:2-[8-amino-6-(3-cyanophenyl)-5-(quinolin-6-yl)imidazo[1,2-a]pyrazin-2-yl]acetamide

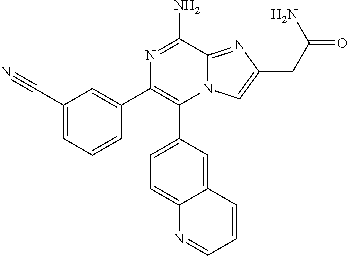 [SMILES: N#Cc1cccc(-c2nc(N)c3nc(CC(N)=O)cn3c2-c2ccc3ncccc3c2)c1]

The title compound was synthesized following the approach outlined in Procedure B, Conditions B in step (c) substituting 3-cyanophenylboronic acid for phenylboronic acid in step (b) and substituting ethyl 4-chloroacetoacetate for chloroacetaldehyde in step (e) and performing this step in DME as a solvent at 85° C. for 16 h and substituting (quinolin-6-yl)boronic acid for (3-chloro-4-hydroxyphenyl)-boronic acid in step (c) and substituting 0.5 M NH₃ in dioxane for ammonium in water in step (f) and heating with 7N NH₃ in methanol at 100° C. for 6 h at this step (f) to lead to the title compound (5.5 mg, 10%) as an off-white solid. ESI-MS: 420.30 [M+H]+. 1H NMR (400 MHz, DMSO-d6) δ 8.98 (dd, J=4.3, 1.7 Hz, 1H), 8.36 (d, J=7.8 Hz, 1H), 8.20-8.00 (m, 2H), 7.83-7.70 (m, 2H), 7.67-7.55 (m, 2H), 7.51 (d, J=8.3 Hz, 1H), 7.38 (s, 1H), 7.37-7.29 (m, 2H), 7.22 (s, 2H), 6.94 (s, 1H), 3.51 (s, 2H).

### Example 168: 6-(4-fluorophenyl)-5-(2-methylquinolin-6-yl)imidazo[1,2-a]pyrazin-8-amine

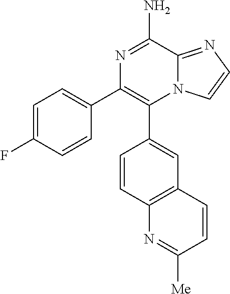 [SMILES: Cc1ccc2cc(-c3c(-c4ccc(F)cc4)nc(N)c4nccn34)ccc2n1]

A) (2-Methylquinolin-6-yl)boronic acid was synthesized following the approach outlined in Procedure A2, substituting 6-bromo-2-methylquinoline for 5-bromo-1H-indazole, using 1,4-dioxane for DMF and heating reaction mixture for 5 h at 80° C. The reaction mixture was filtered through Celite® and the filtrate was concentrated under reduced pressure. The residue was used without further purification. ESI-MS: 187.90 [M+H]+

B) The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (2-methylquinolin-6-yl)boronic acid for 5-(tetramethyl-1,3,2-dioxaborolan-2-yl)-1H-indazole in step (d) and using the conditions B described in Procedure D heating at 140° C. for 3.5 h to lead to the title compound (68 mg, 42%) as a hydrochloride salt as a yellow solid. ESI-MS: 370.2 [M+H]+ 0.1H NMR (400 MHz, DMSO-d6) δ 8.72 (s, 1H), 8.33-8.21 (m, 2H), 7.93-7.84 (m, 2H), 7.80 (d, J=8.6 Hz, 1H), 7.70 (d, J=1.3 Hz, 1H), 7.44-7.36 (m, 2H), 7.18-7.09 (m, 2H), 2.88 (s, 3H).

### Example 169: 3-[8-amino-5-(8-aminoquinolin-6-yl)imidazo[1,2-a]pyrazin-6-yl]benzonitrile

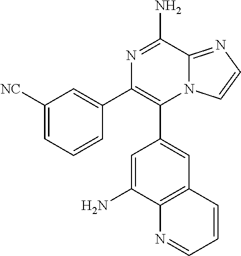 [SMILES: N#Cc1cccc(-c2nc(N)c3nccn3c2-c2cc(N)c3ncccc3c2)c1]

A) (8-Aminoquinolin-6-yl)boronic acid was synthesized following the approach outlined in Procedure A2 substituting 6-bromoquinolin-8-amine for 5-bromo-1H-indazole using instead of DMF 1,4-dioxane, heating the reaction mixture at 80° C. The reaction mixture was filtered through Celite® and the filtrate was concentrated under reduced pressure. The residue was used without further purification. ESI-MS: 188.5 [M+H]+.

B) The title compound was synthesized following the approach outlined in Procedure D, substituting 6-chloro-5-(3-cyanophenyl)pyrazin-2-amine [Procedure B, step (b) substituting 3-cyanophenylboronic acid for phenylboronic acid] for 6-chloro-5-phenylpyrazin-2-amine in step (a) and substituting (8-aminoquinolin-6-yl)boronic acid for 5-(tetramethyl-1,3,2-dioxaborolan-2-yl)-1H-indazole in step (d) and using the conditions B described in Procedure D heating at 130° C. for 2 h to lead to the title compound (5 mg, 57%) as a hydrochloride salt as an orange solid. ESI-MS: 378.8 [M+H]+. 1H NMR (400 MHz, DMSO-d6) δ 8.82 (s, 1H), 8.21 (d, J=8.4 Hz, 1H), 7.91 (s, 1H), 7.77 (s, 1H), 7.73 (d, J=7.8 Hz, 1H), 7.63 (dt, J=8.0, 1.4 Hz, 2H), 7.58-7.51 (m, 1H), 7.47-7.40 (m, 1H), 7.16 (s, 1H), 6.88-6.80 (m, 1H).

### Example 170:6-(4-fluorophenyl)-5-(3-fluoroquinolin-6-yl)imidazo[1,2-a]pyrazin-8-amine

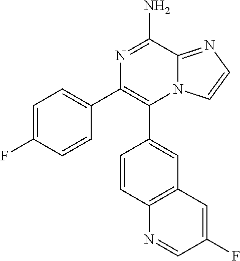 [SMILES: Nc1nc(-c2ccc(F)cc2)c(-c2ccc3ncc(F)cc3c2)n2ccnc12]

A) (3-Fluoroquinolin-6-yl)boronic acid was synthesized following the approach outlined in Procedure A2 substituting 6-bromo-3-fluoroquinoline for 5-bromo-1H-indazole using instead of DMF 1,4-dioxane, heating the reaction mixture for 5 h at 80° C. The reaction mixture was filtered through Celite® and the filtrate was concentrated under reduced pressure to give a dark brown solid (quant.). ESI-MS: 192.15 [M+H]+.

B) The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (3-fluoroquinolin-6-yl)boronic acid for 5-(tetramethyl-1,3,2-dioxaborolan-2-yl)-1H-indazole in step (d) and using the conditions B described in Procedure D heating at 140° C. for 3 h to lead to the title compound (38 mg, 52%) as a hydrochloride salt as a yellow solid. ESI-MS: 374.2 [M+H]+. 1H NMR (400 MHz, DMSO-d6) δ 9.04 (d, J=2.8 Hz, 1H), 8.67 (s, 1H), 8.30 (dd, J=9.4, 2.9 Hz, 1H), 8.14 (d, J=8.7 Hz, 1H), 8.11 (d, J=1.9 Hz, 1H), 7.84 (d, J=1.3 Hz, 1H), 7.72-7.67 (m, 2H), 7.44-7.36 (m, 2H), 7.19-7.09 (m, 2H).

### Example 171:5-(3,5-dichloro-4-methoxyphenyl)-6-phenylimidazo[1,2-a]pyrazin-8-amine

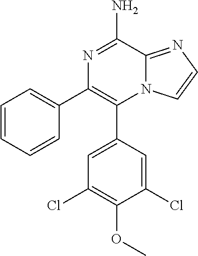 [SMILES: COc1c(Cl)cc(-c2c(-c3ccccc3)nc(N)c3nccn23)cc1Cl]

The title compound was synthesized following the approach outlined in Procedure B, Conditions B in step (b) and substituting (3,5-dichloro-4-methoxyphenyl)boronic acid for (3-chloro-4-hydroxyphenyl)-boronic acid in step (c) to lead to the title compound as a hydrochloride salt (15 mg, 22%) as a yellow solid. ESI-MS: 385.7 [M+H]+. 1H NMR (300 MHz, DMSO-d6) δ 7.86 (dd, J=24.5, 1.3 Hz, 2H), 7.59 (s, 2H), 7.41-7.35 (m, 5H), 3.86 (s, 3H).

### Example 172: 6-(4-fluorophenyl)-5-(2-fluoroquinolin-6-yl)imidazo[1,2-a]pyrazin-8-amine

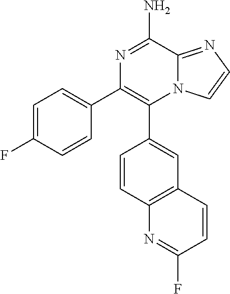 [SMILES: Nc1nc(-c2ccc(F)cc2)c(-c2ccc3nc(F)ccc3c2)n2ccnc12]

A) (2-Fluoroquinolin-6-yl)boronic acid was synthesized following the approach outlined in Procedure A2 substituting 6-bromo-2-fluoroquinoline for 5-bromo-1H-indazole using instead of DMF 1,4-dioxane and heating the reaction mixture for 5 h at 80° C. The reaction mixture was filtered through Celite® and the filtrate was concentrated under reduced pressure to give a dark brown solid (quant). ESI-MS: 192.15 [M+H]+

B) The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (2-fluoroquinolin-6-yl)boronic acid for 5-(tetramethyl-1,3,2-dioxaborolan-2-yl)-1H-indazole in step (d) and using the conditions B described in Procedure D heating at 140° C. for 3 h to lead to the title compound (48 mg, 49%) as a hydrochloride salt as a yellow solid. ESI-MS: 374.2 [M+H]+. 1H NMR (400 MHz, DMSO-d6) δ 8.78 (s, 1H), 8.61 (t, J=8.6 Hz, 1H), 8.18 (d, J=1.9 Hz, 1H), 7.96 (d, J=8.7 Hz, 1H), 7.85 (d, J=1.3 Hz, 1H), 7.76 (dd, J=8.7, 2.0 Hz, 1H), 7.69 (d, J=1.3 Hz, 1H), 7.47-7.36 (m, 3H), 7.21-7.10 (m, 2H).

### Example 173: methyl 5-[8-amino-6-(4-fluorophenyl)imidazo[1,2-a]pyrazin-5-yl]furan-2-carboxylate

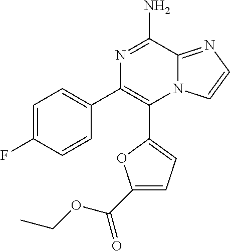 [SMILES: CCOC(=O)c1ccc(-c2c(-c3ccc(F)cc3)nc(N)c3nccn23)o1]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting [5-(methoxycarbonyl)furan-2-yl]boronic acid for 5-(tetramethyl-1,3,2-dioxaborolan-2-yl)-1H-indazole in step (d) and using the conditions B described in Procedure D except substituting Pd Xphos G1 for Pd(amphos)Cl₂ and KF for K₂CO₃ and MeOH/toluene 1:1 for dioxane heating under microwave irradiation at 130° C. for 1 h to lead to the title compound (13 mg, 22%) as a hydrochloride salt as a yellow solid. ESI-MS: 354.1 [M+H]+. 1H NMR (400 MHz, DMSO-d6) δ 8.32 (s, 2H), 7.92 (s, 1H), 7.83 (s, 1H), 7.47-7.39 (m, 2H), 7.37 (d, J=3.6 Hz, 1H), 7.24 (t, J=8.7 Hz, 2H), 6.56 (d, J=3.7 Hz, 1H), 3.83 (s, 3H).

### Example 174: 5-[8-amino-6-(4-fluorophenyl)imidazo[1,2-a]pyrazin-5-yl]-1,2-dihydropyridin-2-one

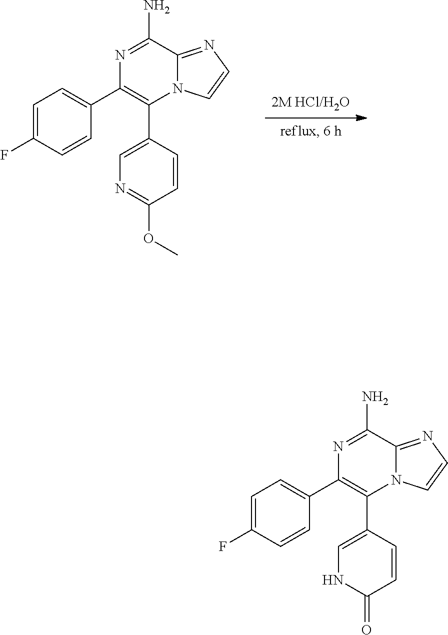 [SMILES: COc1ccc(-c2c(-c3ccc(F)cc3)nc(N)c3nccn23)cn1 ; Nc1nc(-c2ccc(F)cc2)c(-c2ccc(=O)[nH]c2)n2ccnc12]

A) 6-(4-Fluorophenyl)-5-(6-methoxypyridin-3-yl)imidazo[1,2-a]pyrazin-8-amine was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting 6-methoxypyridine-3-boronic acid for 5-(tetramethyl-1,3,2-dioxaborolan-2-yl)-1H-indazole in step (d) and using the conditions A described in Procedure D heating at 150° C. for 18 h to lead to the 6-(4-fluorophenyl)-5-(6-methoxypyridin-3-yl)imidazo[1,2-a]pyrazin-8-amine (48 mg, 38%) as a beige solid. ESI-MS: 337.0 [M+H]+.

B) In a flask equipped with condenser was suspended 6-(4-fluorophenyl)-5-(6-methoxypyridin-3-yl)imidazo[1,2-a]pyrazin-8-amine (39 mg, 0.15 mmol) in 2M aqueous HCl (10 mL) and heated at reflux for 6 h. After cooling to room temperature, the crude reaction mixture was concentrated under reduced pressure. The resulting residue was purified by column chromatography eluting with dichloromethane/methanol. The title product 5-[8-amino-6-(4-fluorophenyl)imidazo[1,2-a]pyrazin-5-yl]-1,2-dihydropyridin-2-one was obtained as a hydrochloride salt as a white solid (25 mg, 41%). ESI-MS: 322.1 [M+H]+. 1H NMR (400 MHz, Deuterium Oxide) δ 7.75 (d, J=1.3 Hz, 1H), 7.72 (d, J=1.3 Hz, 1H), 7.61 (dd, J=9.4, 2.6 Hz, 1H), 7.56 (dd, J=2.6, 0.8 Hz, 1H), 7.41-7.36 (m, 2H), 7.15-7.09 (m, 2H), 6.61 (dd, J=9.4, 0.8 Hz, 1H).

### Example 175: 3-[8-amino-5-(3-aminoquinolin-6-yl)imidazo[1,2-a]pyrazin-6-yl]benzonitrile

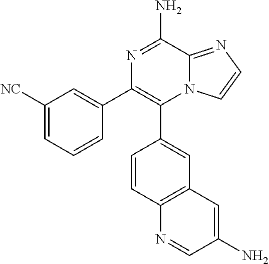 [SMILES: N#Cc1cccc(-c2nc(N)c3nccn3c2-c2ccc3ncc(N)cc3c2)c1]

The title compound was synthesized following the approach outlined in Procedure D, substituting 6-chloro-5-(3-cyanophenyl)pyrazin-2-amine [Procedure B, step (b) substituting 3-cyanophenylboronic acid for phenylboronic acid] for 6-chloro-5-phenylpyrazin-2-amine in step (a) and substituting (3-aminoquinolin-6-yl)boronic acid for 5-(tetramethyl-1,3,2-dioxaborolan-2-yl)-1H-indazole in step (d) and using the conditions B described in Procedure D to lead to the title compound (19 mg, 57%) as a hydrochloride salt as a yellow solid. ESI-MS: 335.2 [M+H]+. 1H NMR (400 MHz, DMSO-d6) δ 9.32 (s, 1H), 8.84 (s, 2H), 8.08-7.87 (m, 3H), 7.49-7.36 (m, 1H), 7.33-7.24 (m, 2H), 7.24-7.08 (m, 2H), 6.73 (dd, J=6.6, 1.4 Hz, 1H), 2.97 (s, 3H).

### Example 176:3-(8-amino-5-{3-methylimidazo[1,2-a]pyridin-6-yl}imidazo[1,2-a]pyrazin-6-yl)benzonitrile

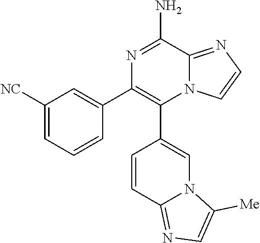 [SMILES: Cc1cnc2ccc(-c3c(-c4cccc(C#N)c4)nc(N)c4nccn34)cn12]

The title compound was synthesized following the approach outlined in Procedure D, substituting 6-chloro-5-(3-cyanophenyl)pyrazin-2-amine [Procedure B, step (b) substituting 3-cyanophenylboronic acid for phenylboronic acid] for 6-chloro-5-phenylpyrazin-2-amine in step (a) and substituting 3-methyl-6-(4,4,5,5-tetramethyl-1,3,2-dioxaborolan-2-yl)imidazo[1,2-a]pyridine for 5-(tetramethyl-1,3,2-dioxaborolan-2-yl)-1H-indazole in step (d) and using the conditions B described in Procedure D to lead to the title compound (80 mg, 11%) as a hydrochloride salt as a white solid. ESI-MS: 366.6 [M+H]+. 1H NMR (300 MHz, DMSO-d6) δ 9.11 (s, 1H), 8.08 (d, J=1.3 Hz, 1H), 8.00 (d, J=9.2 Hz, 1H), 7.93 (dd, J=1.7 Hz, 1H), 7.86-7.72 (m, 4H), 7.63 (ddd, J=7.9, 1.5 Hz, 1H), 7.49-7.39 (m, 1H).

### Example 177: 3-[8-amino-5-(5-fluoroquinolin-6-yl)imidazo[1,2-a]pyrazin-6-yl]benzonitrile

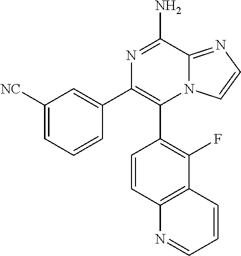 [SMILES: N#Cc1cccc(-c2nc(N)c3nccn3c2-c2ccc3ncccc3c2F)c1]

A) (5-Fluoroquinolin-6-yl)boronic acid was synthesized following the approach outlined in Procedure A2 substituting 6-bromo-5-fluoroquinoline for 5-bromo-1H-indazole using 1,4-dioxane instead of DMF heating reaction mixture at 80° C. for 20 h. The reaction mixture was filtered through Celite® and the filtrate was concentrated under reduced pressure. The residue was used without further purification. ESI-MS: 192.2 [M+H]+.

B) The title compound was synthesized following the approach outlined in Procedure D, substituting 6-chloro-5-(3-cyanophenyl)pyrazin-2-amine [Procedure B, step (b) substituting 3-cyanophenylboronic acid for phenylboronic acid] for 6-chloro-5-phenylpyrazin-2-amine in step (a) and substituting (5-fluoroquinolin-6-yl)boronic acid for 5-(tetramethyl-1,3,2-dioxaborolan-2-yl)-1H-indazole in step (d) and using the conditions B described in Procedure D heating at 150° C. for 1 h to lead to the title compound (3 mg, 3%) as a hydrochloride salt as a yellow solid. ESI-MS: 381.2 [M+H]+. 1H NMR (300 MHz, DMSO-d6) δ 9.08 (dd, J=4.2, 1.7 Hz, 1H), 8.57-8.51 (m, 1H), 7.96 (d, J=8.8 Hz, 1H), 7.86-7.82 (m, 1H), 7.79-7.64 (m, 5H), 7.58-7.52 (m, 1H), 7.45-7.36 (m, 1H).

### Example 178:6-[8-amino-6-(4-fluorophenyl)imidazo[1,2-a]pyrazin-5-yl]imidazo[1,2-a]pyridine-3-carbonitrile

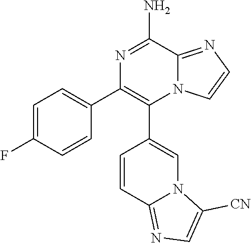 [SMILES: N#Cc1cnc2ccc(-c3c(-c4ccc(F)cc4)nc(N)c4nccn34)cn12]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting 6-(4,4,5,5-tetramethyl-1,3,2-dioxaborolan-2-yl)imidazo[1,2-a]pyridine-3-carbonitrile for 5-(tetramethyl-1,3,2-dioxaborolan-2-yl)-1H-indazole in step (d) and using the conditions A described in Procedure D to lead to the title compound (11 mg, 21%) as a hydrochloride salt as a white solid. ESI-MS: 371.0 [M+H]+. 1H NMR (400 MHz, DMSO-d6) δ 8.95 (s, 1H), 8.54 (s, 1H), 8.07 (d, J=1.4 Hz, 1H), 7.95 (d, J=1.4 Hz, 1H), 7.89 (dd, J=9.2, 1.0 Hz, 1H), 7.43 (dd, J=9.3, 1.7 Hz, 1H), 7.40-7.29 (m, 2H), 7.25-7.15 (m, 2H).

### Example 179: 5-[8-amino-6-(4-fluorophenyl)imidazo[1,2-a]pyrazin-5-yl]-1-methyl-1,2-dihydropyridin-2-one

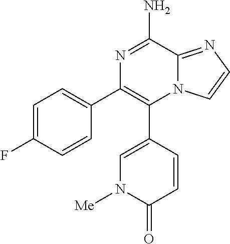 [SMILES: Cn1cc(-c2c(-c3ccc(F)cc3)nc(N)c3nccn23)ccc1=O]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting 1-methyl-5-(4,4,5,5-tetramethyl-1,3,2-dioxaborolan-2-yl)pyridin-2-one for 5-(tetramethyl-1,3,2-dioxaborolan-2-yl)-1H-indazole in step (d) and using the conditions A described in Procedure D heating at 120° C. for 12 h to lead to the title compound (12 mg, 13%) as a hydrochloride salt as a beige solid. ESI-MS: 336.2 [M+H]+. 1H NMR (400 MHz, DMSO-d6) δ 8.02 (s, 1H), 7.94 (s, 1H), 7.91 (d, J=2.6 Hz, 1H), 7.53-7.44 (m, 2H), 7.28 (td, J=9.2, 2.6 Hz, 3H), 6.42 (d, J=9.4 Hz, 1H), 3.39 (s, 3H).

### Example 180: 5-[8-amino-6-(3-fluorophenyl)imidazo[1,2-a]pyrazin-5-yl]-1-methyl-1,2-dihydropyridin-2-one

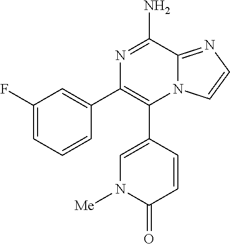 [SMILES: Cn1cc(-c2c(-c3cccc(F)c3)nc(N)c3nccn23)ccc1=O]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 1-methyl-5-(4,4,5,5-tetramethyl-1,3,2-dioxaborolan-2-yl)pyridin-2-one for 5-(tetramethyl-1,3,2-dioxaborolan-2-yl)-1H-indazole in step (d) and using the conditions A described in Procedure D heating at 120° C. for 12 h to lead to the title compound (22 mg, 16%) as a hydrochloride salt as a beige solid. ESI-MS: 336.2 [M+H]+. 1H NMR (400 MHz, DMSO-d6) δ 8.03 (d, J=1.3 Hz, 1H), 7.96 (d, J=1.4 Hz, 1H), 7.92 (d, J=2.6 Hz, 1H), 7.47 (td, J=8.0, 6.1 Hz, 1H), 7.35-7.23 (m, 4H), 6.44 (d, J=9.4 Hz, 1H), 3.40 (s, 3H).

### Example 181:6-[8-amino-6-(3-fluorophenyl)imidazo[1,2-a]pyrazin-5-yl]imidazo[1,2-a]pyridine-3-carbonitrile

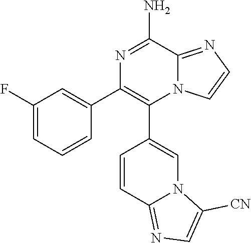 [SMILES: N#Cc1cnc2ccc(-c3c(-c4cccc(F)c4)nc(N)c4nccn34)cn12]

The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting 6-(4,4,5,5-tetramethyl-1,3,2-dioxaborolan-2-yl)imidazo[1,2-a]pyridine-3-carbonitrile for 5-(tetramethyl-1,3,2-dioxaborolan-2-yl)-1H-indazole in step (d) and using the conditions A described in Procedure D to lead to the title compound (20 mg, 45%) as a hydrochloride salt as a white solid. ESI-MS: 370.2 [M+H]+, 1H NMR (400 MHz, DMSO-d6) δ 8.95 (s, 1H), 8.54 (s, 1H), 8.07 (d, J=1.4 Hz, 1H), 7.95 (d, J=1.4 Hz, 1H), 7.89 (dd, J=9.2, 1.0 Hz, 1H), 7.43 (dd, J=9.3, 1.7 Hz, 1H), 7.40-7.29 (m, 2H), 7.25-7.15 (m, 2H).

### Example 182: 5-(4,8-dimethylquinolin-6-yl)-6-(4-fluorophenyl)imidazo[1,2-a]pyrazin-8-amine

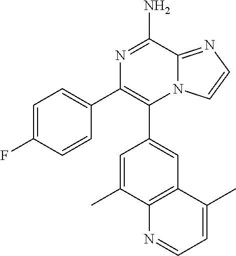 [SMILES: Cc1ccnc2c(C)cc(-c3c(-c4ccc(F)cc4)nc(N)c4nccn34)cc12]

A) (4,8-Dimethylquinolin-6-yl)boronic acid was synthesized following the approach outlined in Procedure A2 substituting 6-bromo-4,8-dimethylquinoline for 6-bromo-4,8-dimethylquinoline and using 1,4-dioxane instead of DMF. The product was obtained as a white solid. ESI-MS: 201.9 [M+H]+.

B) The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (4,8-dimethylquinolin-6-yl)boronic acid for 5-(tetramethyl-1,3,2-dioxaborolan-2-yl)-1H-indazole in step (d) and using the conditions B described in Procedure D heating at 100° C. for 6 h to lead to the title compound (52 mg, 27%) as a hydrochloride salt as a yellow solid. ESI-MS: 384.3 [M+H]+. 1H NMR (400 MHz, DMSO-d6) δ 8.81 (d, J=4.3 Hz, 1H), 7.92 (d, J=1.8 Hz, 1H), 7.62 (d, J=1.7 Hz, 1H), 7.57-7.50 (m, 2H), 7.43-7.39 (m, 1H), 7.35 (dd, J=8.7, 5.7 Hz, 1H), 7.15 (s, 1H), 7.01 (t, J=8.9 Hz, 2H), 2.09 (s, 2H), 1.24 (s, 2H).

### Example 183: 5-(1H-1,3-benzodiazol-6-yl)-6-(4-fluorophenyl)imidazo[1,2-a]pyrazin-8-amine

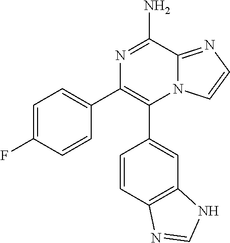 [SMILES: Nc1nc(-c2ccc(F)cc2)c(-c2ccc3nc[nH]c3c2)n2ccnc12]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (1H-benzimidazol-6-yl)boronic acid for 5-(tetramethyl-1,3,2-dioxaborolan-2-yl)-1H-indazole in step (d) and using the conditions B described in Procedure D to lead to the title compound (48 mg, 76%) as a hydrochloride salt as a white solid. ESI-MS: 370.2 [M+H]+, 1H NMR (400 MHz, DMSO-d6) δ 9.42 (s, 1H), 7.91 (d, J=8.4 Hz, 2H), 7.86 (d, J=1.3 Hz, 1H), 7.59 (d, J=1.3 Hz, 1H), 7.52 (dd, J=8.4, 1.6 Hz, 1H), 7.43-7.35 (m, 2H), 7.19-7.09 (m, 2H).

### Example 184: 6-(4-fluorophenyl)-5-{3-methylimidazo[1,2-a]pyridin-6-yl}imidazo[1,2-a]pyrazin-8-amine

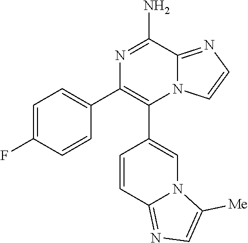 [SMILES: Cc1cnc2ccc(-c3c(-c4ccc(F)cc4)nc(N)c4nccn34)cn12]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting 3-methyl-6-(4,4,5,5-tetramethyl-1,3,2-dioxaborolan-2-yl)Imidazo[1,2-a]pyridine for 5-(tetramethyl-1,3,2-dioxaborolan-2-yl)-1H-indazole in step (d) and using the conditions A described in Procedure D to lead to the title compound (13 mg, 16%) as a hydrochloride salt as a beige solid. ESI-MS: 359.2 [M+H]+. 1H NMR (400 MHz, DMSO-d6) δ 9.14 (t, J=1.2 Hz, 1H), 8.65 (s, 2H), 8.09 (d, J=1.3 Hz, 1H), 8.04-7.95 (m, 2H), 7.91 (d, J=1.3 Hz, 1H), 7.70 (dd, J=9.3, 1.5 Hz, 1H), 7.54-7.44 (m, 2H), 7.17 (t, J=8.8 Hz, 2H), 2.54 (d, J=1.1 Hz, 3H).

### Example 185: 6-(4-fluorophenyl)-5-(4-methoxy-1H-1,3-benzodiazol-6-yl)imidazo[1,2-a]pyrazin-8-amine

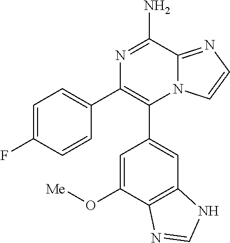 [SMILES: COc1cc(-c2c(-c3ccc(F)cc3)nc(N)c3nccn23)cc2[nH]cnc12]

A) 4-Methoxy-6-(4,4,5,5-tetramethyl-1,3,2-dioxaborolan-2-yl)-1H-1,3-benzodiazole was synthesized following the approach outlined in Procedure A2 substituting 6-bromo-4-methoxy-1H-1,3-benzodiazol for 5-bromo-1H-indazole using 1,4-dioxane instead of DMF and heating at 105° C. for 5 h. The reaction mixture was filtered through Celite® and the filtrate was concentrated under reduced pressure to give a dark brown solid (quant.) which was used in next step without further purification. ESI-MS: 273.9 [M−H]−.

B) The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting 4-methoxy-6-(4,4,5,5-tetramethyl-1,3,2-dioxaborolan-2-yl)-1H-1,3-benzodiazole for 5-(tetramethyl-1,3,2-dioxaborolan-2-yl)-1H-indazole in step (d) and using the conditions B described in Procedure D to lead to the title compound (27 mg, 17%) as a hydrochloride salt as a beige solid. ESI-MS: 375.3 [M+H]+. 1H NMR (400 MHz, Deuterium Oxide) δ 9.08 (s, 1H), 7.79 (d, J=1.2 Hz, 1H), 7.67 (d, J=1.2 Hz, 1H), 7.50 (d, J=0.9 Hz, 1H), 7.47-7.39 (m, 2H), 7.12-7.04 (m, 3H), 3.92 (s, 3H).

### Example 186: 6-(4-fluorophenyl)-5-(3-methylquinolin-6-yl)imidazo[1,2-a]pyrazin-8-amine

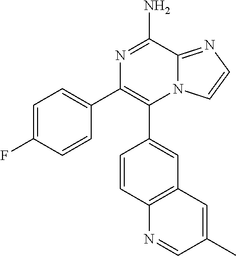 [SMILES: Cc1cnc2ccc(-c3c(-c4ccc(F)cc4)nc(N)c4nccn34)cc2c1]

A) 3-Methyl-6-(4,4,5,5-tetramethyl-1,3,2-dioxaborolan-2-yl)quinoline was prepared following the approach outlined in Procedure A2, substituting 6-bromo-3-methylquinoline for 5-bromo-1H-indazole and using 1,4-dioxane for DMF and heating for 5 h at 80° C. The reaction mixture was filtered through Celite® and the filtrate was concentrated under reduced pressure to give a dark brown solid (quant.) which was used in next step without further purification. ESI-MS: 187.8 [M+H]+.

B) The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting 3-methyl-6-(4,4,5,5-tetramethyl-1,3,2-dioxaborolan-2-yl)quinoline for 5-(tetramethyl-1,3,2-dioxaborolan-2-yl)-1H-indazole in step (d) and using the conditions B described in Procedure D heating at 135° C. for 3 h to lead to the title compound (66 mg, 51%) as a hydrochloride salt as a yellow solid. ESI-MS: 370.2 [M+H]+. 1H NMR (400 MHz, DMSO-d6) δ 8.95 (d, J=2.0 Hz, 1H), 8.74 (s, 1H), 8.31 (s, 1H), 8.11 (d, J=8.7 Hz, 1H), 8.07 (d, J=1.8 Hz, 1H), 7.85 (d, J=1.2 Hz, 1H), 7.71 (dd, J=8.7, 1.9 Hz, 1H), 7.68 (d, J=1.3 Hz, 1H), 7.43-7.37 (m, 2H), 7.18-7.10 (m, 2H).

### Example 187: 5-[8-amino-6-(4-fluorophenyl)imidazo[1,2-a]pyrazin-5-yl]-1-(difluoromethyl)-1,2-dihydropyridin-2-one

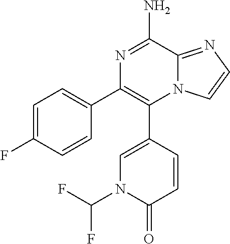 [SMILES: Nc1nc(-c2ccc(F)cc2)c(-c2ccc(=O)n(C(F)F)c2)n2ccnc12]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting 1-(difluoromethyl)-5-(4,4,5,5-tetramethyl-1,3,2-dioxaborolan-2-yl)-2(1H)-pyridinone for 5-(tetramethyl-1,3,2-dioxaborolan-2-yl)-1H-indazole in step (d) and using the conditions B described in Procedure D heating at 135° C. for 3 h to lead to the title compound (59 mg, 24%) as a hydrochloride salt as a beige solid. ESI-MS: 372.2 [M+H]+. 1H NMR 1H NMR (400 MHz, DMSO-d6) δ 8.62 (s, 1H), 7.94 (s, 1H), 7.89 (s, 1H), 7.87 (d, J=2.4 Hz, 1H), 7.81 (s, 1H), 7.57 (dd, J=9.7, 2.4 Hz, 1H), 7.50-7.43 (m, 2H), 7.32-7.24 (m, 2H), 6.63 (d, J=9.6 Hz, 1H).

### Example 188: 1-{4-[8-amino-6-(4-fluorophenyl)imidazo[1,2-a]pyrazin-5-yl]pyridin-2-yl}ethan-1-one

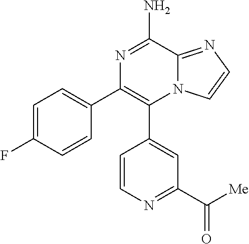 [SMILES: CC(=O)c1cc(-c2c(-c3ccc(F)cc3)nc(N)c3nccn23)ccn1]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting 1-[4-(4,4,5,5-tetramethyl-1,3,2-dioxaborolan-2-yl)pyridin-2-yl]ethan-1-one for 5-(tetramethyl-1,3,2-dioxaborolan-2-yl)-1H-indazole in step (d) and using the conditions B described in Procedure B to lead to the title compound (39 mg, 48%) as a hydrochloride salt as a yellow solid. ESI-MS: 348.2 [M+H]+. 1H NMR (400 MHz, DMSO-d6) δ 8.79 (dd, J=4.9, 0.8 Hz, 1H), 8.42 (s, 1H), 7.96 (dd, J=1.7, 0.9 Hz, 1H), 7.80 (d, J=1.2 Hz, 1H), 7.71 (d, J=1.3 Hz, 1H), 7.63 (dd, J=5.0, 1.7 Hz, 1H), 7.39-7.33 (m 2H), 7.21-7.13 (m 2H), 2.64 (s, 3H).

### Example 189: 5-{8-fluoro-3-methylimidazo[1,2-a]pyridin-6-yl}-6-(3-fluorophenyl)imidazo[1,2-a]pyrazin-8-amine

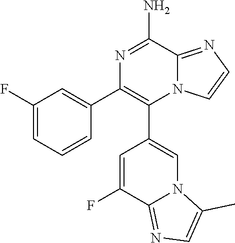 [SMILES: Cc1cnc2c(F)cc(-c3c(-c4cccc(F)c4)nc(N)c4nccn34)cn12]

A) {8-Fluoro-3-methylimidazo[1,2-a]pyridin-6-yl}boronic acid was prepared following the approach outlined in Procedure A2, substituting 6-bromo-8-fluoro-3-methylimidazo[1,2-a]pyridin for 5-bromo-1H-indazole and using 1,4-dioxane for DMF and heating for 6 at 110° C. The reaction mixture was filtered through Celite® and the filtrate was concentrated under reduced pressure to give a purple solid (quant.) which was used in next step without further purification. ESI-MS: 194.8 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting {8-fluoro-3-methylimidazo[1,2-a]pyridin-6-yl}boronic acid for 5-(tetramethyl-1,3,2-dioxaborolan-2-yl)-1H-indazole in step (d) and using the conditions B described in Procedure D except substituting Pd Sphos G3 for Pd(amphos)Cl₂ and heating under microwave irradiation at 110° C. for 1 h to lead to the title compound (8 mg, 10%) as a hydrochloride salt as an off-white solid. ESI-MS: 377.2 [M+H]+. 1H NMR (400 MHz, DMSO-d6) δ 8.65 (s, 1H), 7.95 (d, J=1.4 Hz, 1H), 7.91 (d, J=1.3 Hz, 1H), 7.83 (s, 1H), 7.56 (d, J=11.1 Hz, 1H), 7.39-7.32 (m, 2H), 7.26-7.15 (m, 2H), 2.45 (d, J=1.0 Hz, 3H).

### Example 190:6-(4-fluorophenyl)-5-(4-methylquinazolin-6-yl)imidazo[1,2-a]pyrazin-8-amine

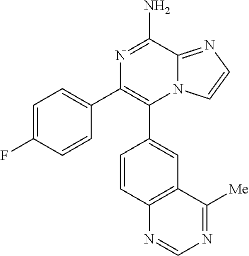 [SMILES: Cc1ncnc2ccc(-c3c(-c4ccc(F)cc4)nc(N)c4nccn34)cc12]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (4-methyl-quinazolin-6-yl)boronic acid for 5-(tetramethyl-1,3,2-dioxaborolan-2-yl)-1H-indazole in step (d) and using the conditions B described in Procedure D heating at 140° C. for 3 h to lead to the title compound (15 mg, 0.04 mmol, 11%) as a beige solid. ESI-MS: 371.9 [M+H]+. 1H NMR (300 MHz, DMSO-d6) δ 9.15 (s, 1H), 8.42 (d, J=1.4 Hz, 1H), 7.97 (d, J=8.6 Hz, 1H), 7.83 (dd, J=8.7, 1.7 Hz, 1H), 7.61-7.59 (m, 2H), 7.56-7.54 (m, 2H), 7.32 (dd, J=8.7, 5.7 Hz, 2H), 7.23 (s, 2H), 7.01 (dd, J=8.9 Hz, 2H), 2.82 (s, 3H).

### Example 191: 4-{8-amino-2-cyclopropyl-6-phenylimidazo[1,2-a]pyrazin-5-yl}-2-chlorophenol

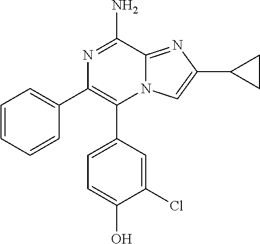 [SMILES: Nc1nc(-c2ccccc2)c(-c2ccc(O)c(Cl)c2)n2cc(C3CC3)nc12]

The title compound was synthesized following the approach outlined in Procedure B, Conditions B in step (c) and substituting 2-bromo-1-cyclopropylethanone for 2-chloroacetaldehyde in step (e) and performing this step in acetonitrile at 100° C. for 1 day to lead to the title compound (white solid, 3 mg, 4%). ESI-MS: 375.1 [M+H]+. 1H NMR (300 MHz, DMSO-d6) δ 7.38-7.15 (m, 7H), 7.11 (dd, J=8.3, 2.1 Hz, 1H), 6.99 (d, J=8.3 Hz, 1H), 6.81 (s, 2H), 4.49-4.37 (m, 1H), 2.06-1.94 (m, 1H), 0.90-0.77 (m, 3H).

### Example 192: 6-[8-amino-6-(4-fluorophenyl)imidazo[1,2-a]pyrazin-5-yl]quinolin-3-amine

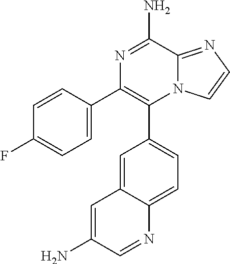 [SMILES: Nc1cnc2ccc(-c3c(-c4ccc(F)cc4)nc(N)c4nccn34)cc2c1]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (3-aminoquinolin-6-yl)boronic acid for 5-(tetramethyl-1,3,2-dioxaborolan-2-yl)-1H-indazole in step (d) using the conditions A described in Procedure D to lead to the title compound (10 mg, 10%) as a hydrochloride salt as a yellow solid. ESI-MS: 371.2 [M+H]+. 1H NMR (400 MHz, DMSO-d6) δ 8.59 (d, J=2.5 Hz, 1H), 7.93 (d, J=8.6 Hz, 1H), 7.88-7.81 (m, 2H), 7.67 (s, 1H), 7.46-7.34 (m, 4H), 7.19-7.10 (m, 2H).

### Example 193: 6-(4-fluorophenyl)-5-{2-methylimidazo[1,2-a]pyridin-6-yl}imidazo[1,2-a]pyrazin-8-amine

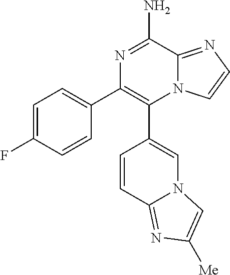 [SMILES: Cc1cn2cc(-c3c(-c4ccc(F)cc4)nc(N)c4nccn34)ccc2n1]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting 2-methyl-7-(4,4,5,5-tetramethyl-1,3,2-dioxaborolan-2-yl)imidazo[1,2-a]pyridine for 5-(tetramethyl-1,3,2-dioxaborolan-2-yl)-1H-indazole in step (d) and using the conditions B described in Procedure D and heating at 140° C. for 3 h to lead to the title compound (33 mg, 22%) as a hydrochloride salt as a white solid. ESI-MS: 359.2. 1H NMR (400 MHz, DMSO-d6) δ 9.06 (s, 1H), 8.95 (s, 1H), 8.17 (s, 1H), 7.98 (d, J=9.3 Hz, 1H), 7.95 (s, 1H), 7.93 (d, J=1.3 Hz, 1H), 7.76 (dd, J=9.3, 1.6 Hz, 1H), 7.49-7.42 (m, 2H), 7.22-7.15 (m, 2H), 2.52 (d, J=1.0 Hz, 3H).

### Example 194: 6-(4-fluorophenyl)-5-{imidazo[1,2-a]pyridin-6-yl}imidazo[1,2-a]pyrazin-8-amine

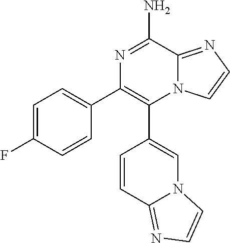 [SMILES: Nc1nc(-c2ccc(F)cc2)c(-c2ccc3nccn3c2)n2ccnc12]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting imidazo[1,2-a]pyridin-6-ylboronic acid for 5-(tetramethyl-1,3,2-dioxaborolan-2-yl)-1H-indazole in step (d) and using the conditions B described in Procedure D except substituting Cs₂CO₃ for K₂CO₃ heating under microwave irradiation at 120° C. for 1.5 h to lead to the title compound (13 mg, 9%) as a hydrochloride salt as a white solid. ESI-MS: 345.1. 1H NMR (400 MHz, DMSO-d6) δ 9.13-9.08 (m, 1H), 8.40 (d, J=2.1 Hz, 1H), 8.26 (d, J=2.1 Hz, 1H), 8.05 (d, J=9.3 Hz, 1H), 7.91-7.85 (m, 2H), 7.81 (dd, J=9.3, 1.6 Hz, 1H), 7.50-7.41 (m, 2H), 7.22-7.13 (m, 2H).

### Example 195: 6-(4-fluorophenyl)-5-(3-fluoropyridin-4-yl)imidazo[1,2-a]pyrazin-8-amine

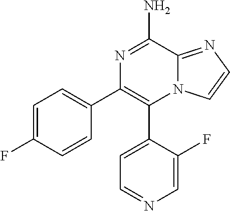 [SMILES: Nc1nc(-c2ccc(F)cc2)c(-c2ccncc2F)n2ccnc12]

The title compound was synthesized following the approach outlined in Procedure B, Conditions B substituting 4-fluorophenylboronic acid for phenylboronic acid in step (b) and heating the reaction for 20 h and substituting (3-fluoropyridin-4-yl)boronic acid for (3-chloro-4-hydroxyphenyl)-boronic acid performing the reaction at 140° C. for 3 h in step (c) to afford the title compound as a hydrochloride salt as a yellow solid (4 mg, 4%). ESI-MS: 324.1 [M+H]+. 1H NMR (400 MHz, DMSO-d6) δ 8.74 (d, J=1.3 Hz, 1H), 8.52 (dd, J=4.9, 1.1 Hz, 1H), 7.86 (d, J=1.3 Hz, 1H), 7.77 (t, J=1.5 Hz, 1H), 7.49 (dd, J=6.1, 4.9 Hz, 1H), 7.42-7.29 (m, 2H), 7.27-7.11 (m, 2H).

### Example 196:6-(3-fluorophenyl)-5-(3-fluoroquinolin-6-yl)imidazo[1,2-a]pyrazin-8-amine

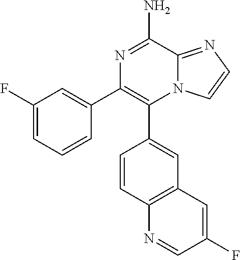 [SMILES: Nc1nc(-c2cccc(F)c2)c(-c2ccc3ncc(F)cc3c2)n2ccnc12]

A) (3-Fluoroquinolin-6-yl)boronic acid was prepared following the approach outlined in Procedure A2, substituting 6-Bromo-3-fluoroquinoline for 5-bromo-1H-indazole and using 1,4-dioxane for DMF and heating under microwave irradiation for 1.5 h at 110° C. The reaction mixture was filtered through Celite® and the filtrate was concentrated under reduced pressure to give a white solid (quant.) which was used in next step without further purification. ESI-MS: 191.9 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting (3-fluoroquinolin-6-yl)boronic acid for 5-(tetramethyl-1,3,2-dioxaborolan-2-yl)-1H-indazole in step (d) and using the conditions A described in Procedure D heating at 130° C. for 5 h to lead to the title compound (6 mg, 6%) as a hydrochloride salt as a beige solid. ESI-MS: 374.2. 1H NMR (400 MHz, DMSO-d6) δ 9.01 (d, J=2.9 Hz, 1H), 8.25 (dd, J=9.5, 2.9 Hz, 1H), 8.17-8.06 (m, 2H), 7.73 (dd, J=8.7, 2.0 Hz, 1H), 7.56 (d, J=1.2 Hz, 1H), 7.47 (d, J=1.2 Hz, 1H), 7.25 (s, 2H), 7.19-7.11 (m, 2H), 7.07-6.95 (m, 2H).

### Example 197: 3-(8-amino-5-{imidazo[1,2-a]pyridin-6-yl}imidazo[1,2-a]pyrazin-6-yl)benzonitrile

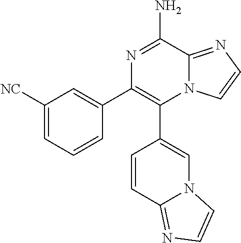 [SMILES: N#Cc1cccc(-c2nc(N)c3nccn3c2-c2ccc3nccn3c2)c1]

The title compound was synthesized following the approach outlined in Procedure D, substituting 6-chloro-5-(3-cyanophenyl)pyrazin-2-amine [Procedure B, step (b) substituting 3-cyanophenylboronic acid for phenylboronic acid] for 6-chloro-5-phenylpyrazin-2-amine in step (a) and substituting imidazo[1,2-a]pyridin-6-ylboronic acid for 5-(tetramethyl-1,3,2-dioxaborolan-2-yl)-1H-indazole in step (d) and using the conditions B described in Procedure D except substituting Pd Sphos G3 for Pd(amphos)Cl₂ and Cs₂CO₃ for K₂CO₃ heating under microwave irradiation at 140° C. for 30 min to lead to the title compound (19 mg, 45%) as a hydrochloride salt as a white solid. ESI-MS: 352.1. 1H NMR (400 MHz, DMSO-d6) δ 9.09 (s, 1H), 8.35 (d, J=2.1 Hz, 1H), 8.24 (d, J=2.1 Hz, 1H), 8.04 (d, J=9.3 Hz, 1H), 7.92-7.82 (m, 2H), 7.82-7.70 (m, 3H), 7.64-7.56 (m, 1H), 7.51-7.36 (m, 1H).

### Example 198: 5-[8-amino-6-(4-fluorophenyl)imidazo[1,2-a]pyrazin-5-yl]-3-methyl-1,2-dihydropyridin-2-one

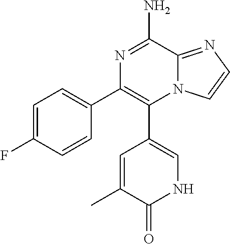 [SMILES: Cc1cc(-c2c(-c3ccc(F)cc3)nc(N)c3nccn23)c[nH]c1=O]

The title compound was synthesized following the approach outlined in Procedure B, Conditions A in step (c) substituting 4-fluorophenylboronic acid for phenylboronic acid in step (b) and substituting Pd(amphos)Cl₂ for Pd(PPh₃)₄ and 6-Hydroxy-5-methylpyridine-3-boronic acid pinacol ester for (3-chloro-4-hydroxyphenyl)-boronic acid in step (c) and performing this reaction for 5 h at 120° C. to lead to the title compound (4.5 mg, 10%) as a brown solid. ESI-MS: 336.10 [M+H]+. 1H NMR (400 MHz, DMSO-d6) δ 7.74 (s, 2H), 7.47-7.41 (m, 2H), 7.37-7.34 (m, 1H), 7.29-7.20 (m, 3H), 1.97 (s, 3H).

### Example 199: 3-[8-amino-5-(1H-1,3-benzodiazol-6-yl)imidazo[1,2-a]pyrazin-6-yl]benzonitrile

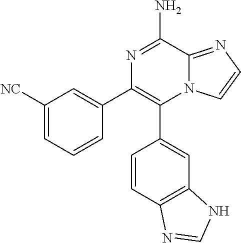 [SMILES: N#Cc1cccc(-c2nc(N)c3nccn3c2-c2ccc3nc[nH]c3c2)c1]

The title compound was synthesized following the approach outlined in Procedure D, substituting 6-chloro-5-(3-cyanophenyl)pyrazin-2-amine [Procedure B, step (b) substituting 3-cyanophenylboronic acid for phenylboronic acid] for 6-chloro-5-phenylpyrazin-2-amine in step (a) and substituting (1H-Benzimidazol-6-yl)boronic acid for 5-(tetramethyl-1,3,2-dioxaborolan-2-yl)-1H-indazole in step (d) and using the conditions B described in Procedure D heating at 150° C. for 3 h to lead to the title compound (30 mg, 34%) as a hydrochloride salt as a beige solid. ESI-MS: 352.4. 1H NMR (300 MHz, DMSO-d6) δ 9.48 (s, 1H), 8.50 (s, 2H), 7.97-7.91 (m, 2H), 7.88-7.83 (m, 2H), 7.78-7.72 (m, 1H), 7.60-7.53 (m, 3H), 7.48-7.41 (m, 1H).

### Example 200: 6-(4-fluorophenyl)-5-{[1,2,4]triazolo[4,3-a]pyridin-6-yl}imidazo[1,2-a]pyrazin-8-amine

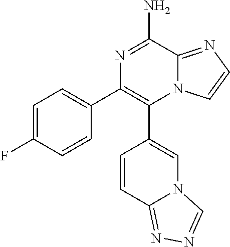 [SMILES: Nc1nc(-c2ccc(F)cc2)c(-c2ccc3nncn3c2)n2ccnc12]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (1,2,4-Triazolo[4,3-a]pyridin-6-yl)boronic acid for 5-(tetramethyl-1,3,2-dioxaborolan-2-yl)-1H-indazole in step (d) and using the conditions A described in Procedure D and heating under microwave irradiation at 140° C. for 30 min to lead to the title compound (18 mg, 22%) as a hydrochloride salt as a brown solid. ESI-MS: 3461. 1H NMR (400 MHz, DMSO-d6) δ 9.39 (s, 1H), 8.81 (s, 1H), 7.98 (d, J=1.1 Hz, 1H), 7.95-7.86 (m, 2H), 7.51-7.44 (m, 2H), 7.34 (dd, J=9.5, 1.4 Hz, 1H), 7.25-7.18 (m, 2H).

### Example 201:5-{3-ethylimidazo[1,2-a]pyridin-6-yl}-6-(4-fluorophenyl)imidazo[1,2-a]pyrazin-8-amine

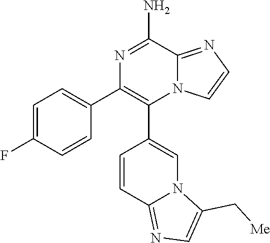 [SMILES: CCc1cnc2ccc(-c3c(-c4ccc(F)cc4)nc(N)c4nccn34)cn12]

A) {3-Ethylimidazo[1,2-a]pyridin-6-yl}boronic acid was prepared following the approach outlined in Procedure A2, substituting 6-bromo-3-ethylimidazo[1,2-a]pyridine for 5-bromo-1H-indazole and using 1,4-dioxane for DMF and heating for 18 h at 90° C. The reaction mixture was filtered through Celite® and the filtrate was concentrated under reduced pressure to give a dark brown solid (quant.) which was used in next step without further purification. ESI-MS: 191.0 [M+H]+.

B) The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting {3-ethylimidazo[1,2-a]pyridin-6-yl}boronic acid for 5-(tetramethyl-1,3,2-dioxaborolan-2-yl)-1H-indazole in step (d) and using the conditions B described in Procedure D and heating at 120° C. for 4 h to lead to the title compound (24 mg, 19%) as a hydrochloride salt as a beige solid. ESI-MS: 324.1. 1H NMR (400 MHz, DMSO-d6) δ 9.11-9.08 (m, 1H), 8.10 (d, J=1.3 Hz, 1H), 8.01 (dd, J=9.3, 0.9 Hz, 1H), 7.88 (s, 1H), 7.81 (s, 1H), 7.78 (dd, J=9.3, 1.5 Hz, 1H), 7.49-7.41 (m, 2H), 7.14 (t, J=8.9 Hz, 2H), 2.97-2.85 (m, 2H), 1.26 (t, J=7.5 Hz, 3H).

### Example 202: 6-(4-fluorophenyl)-5-{pyrazolo[1,5-a]pyridin-5-yl}imidazo[1,2-a]pyrazin-8-amine

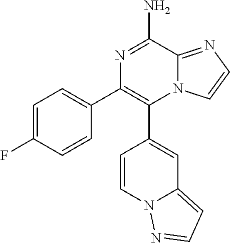 [SMILES: Nc1nc(-c2ccc(F)cc2)c(-c2ccn3nccc3c2)n2ccnc12]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting pyrazolo[1,5-a]pyridine-5-boronic acid for 5-(tetramethyl-1,3,2-dioxaborolan-2-yl)-1H-indazole in step (d) and using the conditions B described in Procedure D except substituting Cs₂CO₃ for K₂CO₃ and heating under microwave irradiation at 130° C. for 1 h to lead to the title compound (16 mg, 25%) as a hydrochloride salt as pink solid. ESI-MS: 345.1 [M+H]+. 1H NMR (400 MHz, DMSO-d6) δ 8.82-8.77 (m, 1H), 8.08 (d, J=2.3 Hz, 1H), 7.92 (d, J=1.4 Hz, 1H), 7.87 (d, J=1.3 Hz, 1H), 7.79 (dd, J=1.9, 1.0 Hz, 1H), 7.51-7.40 (m, 2H), 7.29-7.15 (m, 2H), 6.86 (dd, J=7.2, 1.9 Hz, 1H), 6.72 (dd, J=2.3, 0.9 Hz, 1H).

### Example 203: 5-[8-amino-6-(4-fluorophenyl)imidazo[1,2-a]pyrazin-5-yl]-3-fluoro-1,2-dihydropyridin-2-one

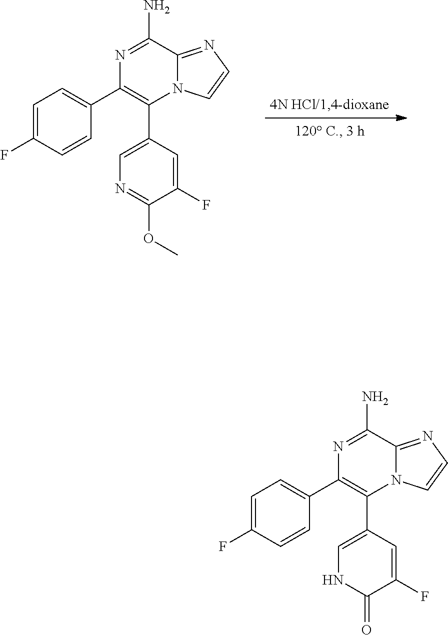 [SMILES: COc1ncc(-c2c(-c3ccc(F)cc3)nc(N)c3nccn23)cc1F ; Nc1nc(-c2ccc(F)cc2)c(-c2c[nH]c(=O)c(F)c2)n2ccnc12]

A) 5-(5-Fluoro-6-methoxypyridin-3-yl)-6-(4-fluorophenyl)imidazo[1,2-a]pyrazin-8-amine was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (5-fluoro-6-methoxypyridin-3-yl)boronic acid for 5-(tetramethyl-1,3,2-dioxaborolan-2-yl)-1H-indazole in step (d) and using the conditions B described in Procedure D except substituting Pd(PPh₃)₄ for Pd(amphos)Cl₂ and heating at 90° C. for 3 h to lead to the title compound (9 mg, 10%) as a white solid. ESI-MS: 354.2 [M+H]+.

B) In a pressure tube was suspended 5-(5-fluoro-6-methoxypyridin-3-yl)-6-(4-fluorophenyl)imidazo[1,2-a]pyrazin-8-amine (9.1 mg, 0.2 mmol) in 4N HCl in 1,4-dioxane (3 mL) and heated at 120° C. for 3 h. After cooling to room temperature, crude reaction mixture was triturated with diethyl ether followed by pentane, precipitates were collected. The title product 5-[8-amino-6-(4-fluorophenyl)imidazo[1,2-a]pyrazin-5-yl]-3-fluoro-1,2-dihydropyridin-2-one was obtained as a hydrochloride salt as a white solid (8 mg, 88%). ESI-MS: 340.1 [M+H 1H NMR (300 MHz, DMSO-d6) δ 12.39 (s, 1H), 7.96 (s, 1H), 7.87 (d, J=1.2 Hz, 1H), 7.55-7.42 (m, 3H), 7.34-7.23 (m, 3H).

### Example 204: 4-{8-amino-5-[2-methyl-6-(trifluoromethyl)pyridin-4-yl]imidazo[1,2-a]pyrazin-6-yl}benzonitrile

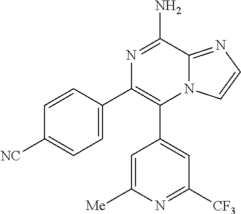 [SMILES: Cc1cc(-c2c(-c3ccc(C#N)cc3)nc(N)c3nccn23)cc(C(F)(F)F)n1]

The title compound was synthesized following the approach outlined in Procedure B, Conditions B substituting 4-cyanophenylboronic acid for phenylboronic acid in step (b) and performing the reaction for 20 h and substituting 2-methyl-4-(tetramethyl-1,3,2-dioxaborolan-2-yl)-6-(trifluoromethyl)pyridine (Procedure A1) for (3-chloro-4-hydroxyphenyl)-boronic acid in step (c) and performing the reaction in step (d) for 20 h and performing the reaction in step (f) at 110° C. under microwave irradiation for 1.5 h to afford the title compound as hydrochloride salt (beige solid, 20 mg, 13%). ESI-MS: 395.3 [M+H]+. 1H NMR (400 MHz, DMSO-d6) δ 8.57 (s, 2H), 7.92 (d, J=16.7 Hz, 2H), 7.83 (d, J=8.1 Hz, 2H), 7.70 (s, 1H), 7.63 (s, 1H), 7.50 (d, J=8.1 Hz, 2H), 2.57 (s, 3H).

### Example 205: 4-[8-amino-5-(8-fluoroquinolin-6-yl)imidazo[1,2-a]pyrazin-6-yl]benzonitrile

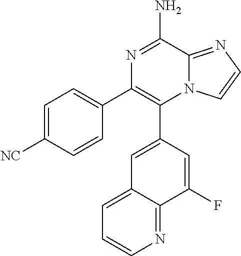 [SMILES: N#Cc1ccc(-c2nc(N)c3nccn3c2-c2cc(F)c3ncccc3c2)cc1]

The title compound was synthesized following the approach outlined in Procedure F (example 207), substituting 4-cyanophenylboronic acid for 3-pyridineboronic acid pinacol ester in step (e) performing this step using Pd(PPh₃)₄ (0.1 equiv.) and Na₂CO₃ (2.5 equiv.) for 12 h to afford the title compound as a hydrochloride salt as a yellow solid (6 mg, 12%). ESI-MS: 381.2 [M+H]+. 1H NMR (400 MHz, DMSO-d6) δ 9.04 (dd, J=4.3, 1.6 Hz, 1H), 8.42 (dt, J=8.5, 1.6 Hz, 1H), 7.89 (d, J=1.7 Hz, 1H), 7.75-7.66 (m, 5H), 7.56-7.50 (m, 2H).

### Example 206:6-(3-fluorophenyl)-5-{1H,2H,3H-pyrrolo[2,3-b]pyridin-4-yl}imidazo[1,2-a]pyrazin-8-amine

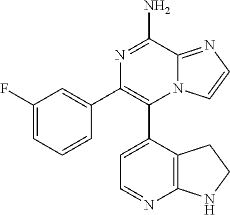 [SMILES: Nc1nc(-c2cccc(F)c2)c(-c2ccnc3c2CCN3)n2ccnc12]

A) {1H,2H,3H-Pyrrolo[2,3-b]pyridin-4-yl}boronic acid was prepared following the approach outlined in Procedure A2, substituting 4-bromo-2,3-dihydro-1H-pyrrolo[2,3-b]pyridine for 5-bromo-1H-indazole and using 1,4-dioxane for DMF and heating for 20 h at 80° C. The reaction mixture was filtered through Celite® and the filtrate was concentrated under reduced pressure to give a brown oil (quant.) which was used in next step without further purification. ESI-MS: 165.15 [M+H]+.

B) The title compound was synthesized following the approach outlined in Procedure D, substituting 6-chloro-5-(3-fluorophenyl)pyrazin-2-amine [Procedure B, step (b) substituting 3-fluorophenylboronic acid for phenylboronic acid] for 6-chloro-5-phenylpyrazin-2-amine in step (a) and substituting {1H,2H,3H-pyrrolo[2,3-b]pyridin-4-yl}boronic acid for 5-(tetramethyl-1,3,2-dioxaborolan-2-yl)-1H-indazole in step (d) and using the conditions B described in Procedure D heating at 120° C. for 5 h to lead to the title compound (4 mg, 4%) as a hydrochloride salt as a yellow solid. ESI-MS: 347.1 [M+H]+. 1H NMR (400 MHz, DMSO-d6) δ 9.03 (s, 1H), 8.03 (s, 2H), 7.84 (s, 1H), 7.79 (s, 1H), 7.63 (d, J=6.8 Hz, 1H), 7.40 (dd, J=14.1, 7.9 Hz, 1H), 7.33-7.10 (m, 3H), 6.55 (d, J=6.7 Hz, 1H), 2.96-2.69 (m, 2H), 2.65-2.53 (m, 2H).

### Procedure F: Example 207: 5-(8-fluoroquinolin-6-yl)-6-(pyridin-3-yl)imidazo[1,2a]pyrazin-8-amine

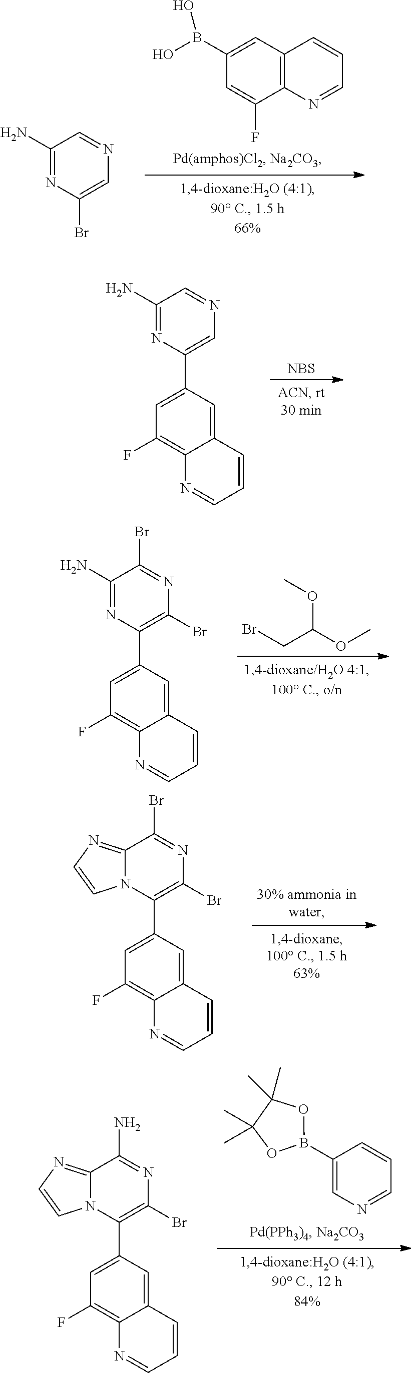

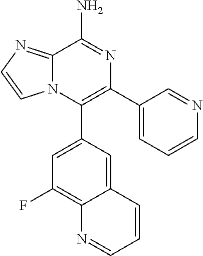 [SMILES: Nc1nc(-c2cccnc2)c(-c2cc(F)c3ncccc3c2)n2ccnc12]

### a. 6-(8-fluoroquinolin-6-yl)pyrazin-2-amine

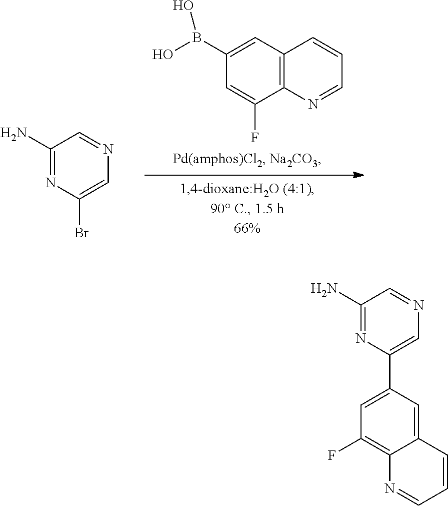 [SMILES: Nc1cncc(Br)n1 ; OB(O)c1cc(F)c2ncccc2c1 ; Nc1cncc(-c2cc(F)c3ncccc3c2)n1]

In a pressure tube were mixed 2-amino-6-bromopyrazine (4.00 g, 22.99 mmol), (8-fluoroquinolin-6-yl)boronic acid (6.98 g, 25.29 mmol, 1.1 equiv.) and Na₂CO₃ (7.31 g, 68.97 mmol, 3 equiv.) in a 4:1 mixture of 1,4-dioxane:water (125 mL). The reaction mixture was sparged with argon and Pd(amphos)Cl₂ (0.814 g, 1.15 mmol, 5 mol %) was then added. The pressure tube was capped and the reaction mixture was heated at 90° C. for 1.5 h. After that time, the reaction mixture was poured on ice then extracted with DCM. Combined organic layers were dried over sodium sulfate, filtrated and concentrated under reduced pressure. The obtained crude material was purified by flash chromatography on silica eluting with hexane:EtOAc=1:0-1:2 to give the title product as a yellow solid (3.69 g, 66%). ESI-MS: 241.20 [M+H]+.

### b. 3,5-dibromo-6-(8-fluoroquinolin-6-yl)pyrazin-2-amine

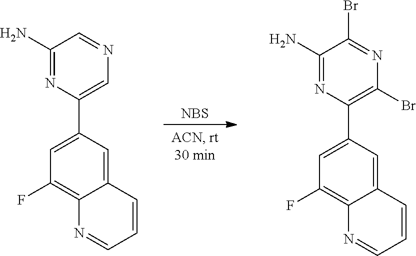 [SMILES: Nc1cncc(-c2cc(F)c3ncccc3c2)n1 ; "NBS" ; Nc1nc(-c2cc(F)c3ncccc3c2)c(Br)nc1Br]

To a solution of 6-(8-fluoroquinolin-6-yl)pyrazin-2-amine (0.490 g, 1.47 mmol) in ACN (4 mL) N-bromosuccinimide (0.574 g, 3.23 mmol, 2.2 equiv.) was added portionwise. The reaction mixture was stirred at r.t. for 1 h (TLC/LC-MS indicated completion of the reaction). Solvent was evaporated. Crude material was purified by flash chromatography on silica eluting with hexane/EtOAc (0-50%) to afford the title product (403 mg, 1.01 mmol, 69%) as a white solid. ESI-MS: 398.89 [M+H]+. 1H NMR (400 MHz, DMSO-d6) δ 9.04 (dd, J=4.2, 1.7 Hz, 1H), 8.57 (dt, J=8.5, 1.6 Hz, 1H), 8.19-8.13 (m, 1H), 7.85 (dd, J=11.7, 1.8 Hz, 1H), 7.72 (dd, J=8.4, 4.2 Hz, 1H), 7.17 (s, 2H).

### c. 6-{6,8-dibromoimidazo[1,2-a]pyrazin-5-yl}-8-fluoroquinoline

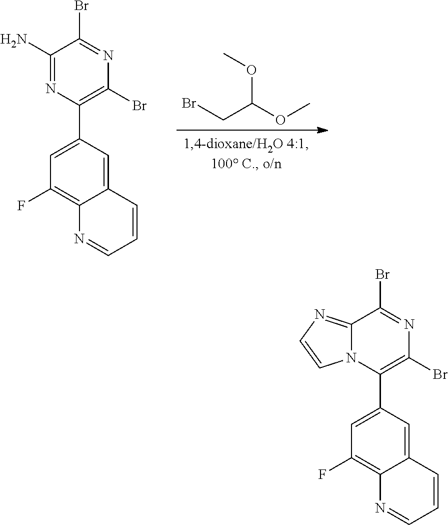

To a suspension of 3,5-dibromo-6-(8-fluoroquinolin-6-yl)pyrazin-2-amine (4.76 g, 11.95 mmol) in mixture of 1,4-dioxane:water (4:1) (70 mL) was added 2-bromo-1,1-dimethoxyethane (2.54 mL, 21.51 mmol, 1.8 equiv.) and the reaction mixture was heated at 100° C. for 2 h. The reaction mixture was then cooled down to r.t., diluted with water and extracted with DCM, organic layers were collected and dried using sodium sulfate. Crude product was concentrated under reduced pressure to lead to the title compound as a beige residue that was used in next step without further purification. ESI-MS: 425.1 [M+H]+.

### d. 6-bromo-5-(8-fluoroquinolin-6-yl)imidazo[1,2-a]pyrazin-8-amine

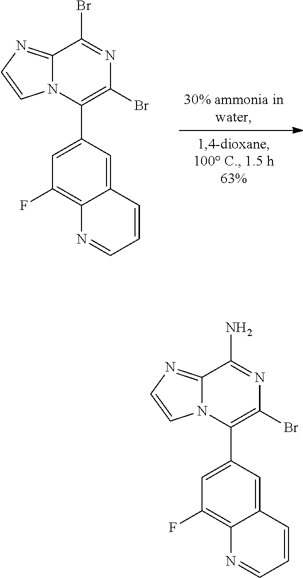 [SMILES: Fc1cc(-c2c(Br)nc(Br)c3nccn23)cc2cccnc12 ; Nc1nc(Br)c(-c2cc(F)c3ncccc3c2)n2ccnc12]

In a pressure tube 6-{6,8-dibromoimidazo[1,2-a]pyrazin-5-yl}-8-fluoroquinoline (130 mg, 0.31 mmol) was dissolved in acetonitrile (1 mL) then 28% aqueous ammonium hydroxide solution (3 mL) was added and the reaction mixture was warmed to 100° C. and stirred for 1.5 h. After that time, the reaction mixture was cooled down to r.t. and concentrated under reduced pressure. The crude material was purified by flash chromatography on silica eluting with DCM/MeOH (0-5%) to afford the title product as a pale yellow solid. ESI-MS: 358.4 [M+H]+.

### e. 5-(8-fluoroquinolin-6-yl)-6-(pyridin-3-yl)imidazo(1,2-a pyrazin-8-amine

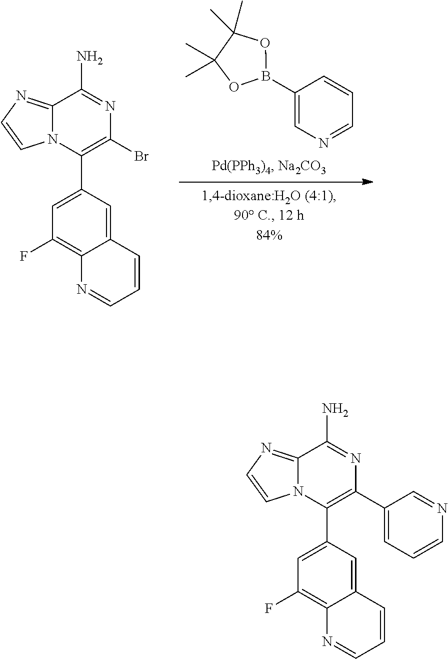 [SMILES: Nc1nc(Br)c(-c2cc(F)c3ncccc3c2)n2ccnc12 ; CC1(C)OB(c2cccnc2)OC1(C)C ; Nc1nc(-c2cccnc2)c(-c2cc(F)c3ncccc3c2)n2ccnc12]

In a pressure tube were mixed 6-bromo-5-(8-fluoroquinolin-6-yl)imidazo[1,2-a]pyrazin-8-amine (50 mg, 0.14 mmol), 3-pyridineboronic acid pinacol ester (37 mg, 0.18 mmol, 1.3 equiv.) and Na₂CO₃ (44 mg, 0.42 mmol, 3 equiv.) in a 4:1 mixture of 1,4-dioxane:water (2.5 mL). The reaction mixture was sparged with argon for 10 min and Pd(PPh₃)₄ (8 mg, 0.01 mmol, 5 mol %) was then added. The pressure tube was capped and the reaction mixture was heated at 90° C. for 12 h. The reaction mixture was then cooled down to r.t., filtered through Celite® and rinsed with DCM. The organic solution was washed with water and brine and aqueous layer was extracted with DCM. Separated organic layer was dried over anhydrous sodium sulfate, filtered and concentrated under reduced pressure to afford the crude material as a brown residue. The obtained crude material was purified by flash chromatography on silica eluting with DCM and MeOH (0-5%) to lead to the title product as a yellow solid. ESI-MS: 357.7 [M+H]+.

The freebase product was dissolved in dry DCM (1 mL) then 2M HCl in diethylether (1 mL) was added. The reaction mixture was stirred for 1 h at r.t. (formation of precipitate occurred during addition of ether solution). The suspension was concentrated under reduced pressure and lyophilized to obtained product as a hydrochloride salt (yellow solid, 42 mg, 0.12 mmol, 84% over 2 steps). ESI-MS: 357.2 [M+H]+ 0.1H NMR (400 MHz, DMSO-d6) δ 9.06 (dd, J=4.2, 1.6 Hz, 1H), 8.74 (d, J=2.1 Hz, 1H), 8.69-8.66 (m, 1H), 8.48-8.43 (m, 1H), 8.15-8.10 (m, 1H), 8.01-7.97 (m, 2H), 7.87 (d, J=1.5 Hz, 1H), 7.79-7.67 (m, 3H).

### Example 208: 6-(2-fluoropyridin-4-yl)-5-(4-methylquinolin-6-yl)imidazo[1,2-a]pyrazin-8-amine

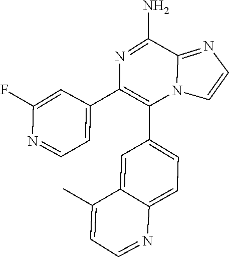 [SMILES: Cc1ccnc2ccc(-c3c(-c4ccnc(F)c4)nc(N)c4nccn34)cc12]

The title compound was synthesized following the approach outlined in Procedure F, substituting (4-methylquinolin-6-yl)boronic acid (example 129) for (8-fluoroquinolin-6-yl)boronic acid in step (a), substituting 2-fluoropyridine-4-boronic acid for 3-pyridineboronic acid pinacol ester in step (e) to afford the title compound as a hydrochloride salt (yellow solid, 12 mg, 77%). ESI-MS: 371.20 [M+H]+. 1H NMR (400 MHz, Deuterium Oxide) δ 8.93 (d, J=5.8 Hz, 1H), 8.50 (d, J=1.7 Hz, 1H), 8.20 (d, J=8.8 Hz, 1H), 8.03-7.97 (m, 2H), 7.89 (dd, J=5.8, 0.9 Hz, 1H), 7.74 (d, J=1.5 Hz, 1H), 7.61 (d, J=1.5 Hz, 1H), 7.20 (dt, J=5.4, 1.6 Hz, 1H), 7.05-7.01 (m, 1H), 2.80 (d, J=0.9 Hz, 3H).

### Example 209: 5-[8-amino-5-(1-methyl-1H-1,3-benzodiazol-6-yl)imidazo[1,2-a]pyrazin-6-yl]-2-fluorobenzonitrile

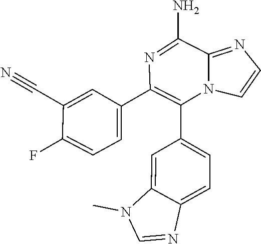 [SMILES: Cn1cnc2ccc(-c3c(-c4ccc(F)c(C#N)c4)nc(N)c4nccn34)cc21]

The title compound was synthesized following the approach outlined in Procedure F, substituting (1-methyl-1H-1,3-benzodiazol-6-yl)boronic acid for (8-fluoroquinolin-6-yl)boronic acid in step (a), then substituting 3-cyano-4-fluorobenzeneboronic acid for 3-pyridineboronic acid pinacol ester in step (e) to afford the title compound as a hydrochloride salt (brown solid, 20 mg, 23.9%). ESI-MS: 371.20 [M+H]+. 1H NMR (400 MHz, DMSO-d6) δ 9.34 (s, 1H), 8.10 (d, J=1.5 Hz, 1H), 7.94-7.87 (m, 2H), 7.77 (d, J=1.3 Hz, 1H), 7.59 (ddd, J=8.9, 5.3, 2.3 Hz, 1H), 7.55 (d, J=1.3 Hz, 1H), 7.47 (dd, J=8.5, 1.5 Hz, 1H), 7.38 (t, J=9.1 Hz, 1H), 3.99 (s, 3H).

### Example 210: 5-[8-amino-6-(5-methylfuran-2-yl)imidazo[1,2-a]pyrazin-5-yl]-1,2-dihydropyridin-2-one

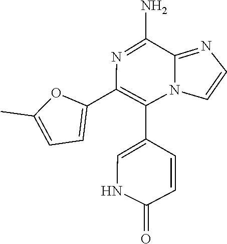 [SMILES: Cc1ccc(-c2nc(N)c3nccn3c2-c2ccc(=O)[nH]c2)o1]

The title compound was synthesized following the approach outlined in Procedure F, substituting 5-(4,4,5,5-tetramethyl-1,3,2-dioxaborolan-2-yl)-1,2-dihydropyridin-2-one for (8-fluoroquinolin-6-yl)boronic acid in step (a), then substituting (5-methylfuran-2-yl)boronic acid for 3-pyridineboronic acid in step (e) to afford the title compound as a hydrochloride salt (orange solid, 14 mg, 15%). ESI-MS: 308.1 [M+H]+. 1H NMR (400 MHz, DMSO-d6) δ 7.89 (s, 1H), 7.85 (d, J=1.4 Hz, 1H), 7.64 (d, J=2.6 Hz, 1H), 7.45 (dd, J=9.5, 2.6 Hz, 1H), 6.53 (d, J=9.5 Hz, 1H), 6.43 (d, J=3.4 Hz, 1H), 6.25-6.21 (m, 1H), 2.25 (s, 3H).

### Example 211:5-[8-amino-6-(1-methyl-1H-pyrazol-3-yl)imidazo[1,2-a]pyrazin-5-yl]-1,2-dihydropyridin-2-one

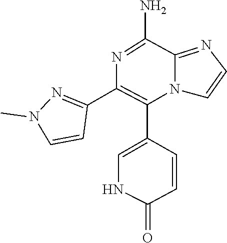 [SMILES: Cn1ccc(-c2nc(N)c3nccn3c2-c2ccc(=O)[nH]c2)n1]

The title compound was synthesized following the approach outlined in Procedure F, substituting 5-(4,4,5,5-tetramethyl-1,3,2-dioxaborolan-2-yl)-1,2-dihydropyridin-2-one for (8-fluoroquinolin-6-yl)boronic acid in step (a), then substituting 1-methyl-3-(4,4,5,5-tetramethyl-1,3,2-dioxaborolan-2-yl)-1H-pyrazole for 3-pyridineboronic acid in step (e) to afford the title compound as a hydrochloride salt (yellow solid, 12.9 mg, 12.8%). ESI-MS: 308.2 1H NMR (400 MHz, DMSO-d6) δ 7.89 (d, J=1.2 Hz, 1H), 7.84 (d, J=1.1 Hz, 1H), 7.80 (d, J=2.4 Hz, 1H), 7.67-7.64 (m, 1H), 7.47 (dd, J=9.5, 2.6 Hz, 1H), 6.57 (d, J=9.4 Hz, 1H), 5.80-5.75 (m, 1H), 3.95 (s, 3H).

### Example 212: 5-[8-amino-5-(4-methylquinolin-6-yl)imidazo[1,2-a]pyrazin-6-yl]-2-fluorobenzonitrile

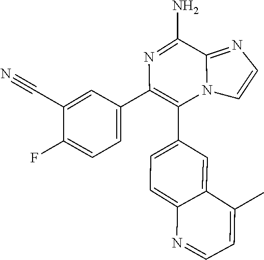 [SMILES: Cc1ccnc2ccc(-c3c(-c4ccc(F)c(C#N)c4)nc(N)c4nccn34)cc12]

The title compound was synthesized following the approach outlined in Procedure F, substituting (4-methylquinolin-6-yl)boronic acid (example 129) for (8-fluoroquinolin-6-yl)boronic acid in step (a), substituting 3-cyano-4-fluorobenzeneboronic acid for 3-pyridineboronic acid pinacol ester in step (e) to afford the title compound as a hydrochloride salt (yellow solid, 16 mg, 81.3%). ESI-MS: 395.20 [M+H]+. 1H NMR (400 MHz, DMSO-d6) δ 8.91 (d, J=5.7 Hz, 1H), 8.45 (d, J=1.7 Hz, 1H), 8.21-8.12 (m, 1H), 7.96 (dd, J=8.8, 1.8 Hz, 1H), 7.86 (dd, J=5.8, 1.0 Hz, 1H), 7.75 (dd, J=5.9, 2.3 Hz, 1H), 7.71 (d, J=1.4 Hz, 1H), 7.60 (qd, J=4.8, 2.4 Hz, 2H), 7.15 (t, J=8.9 Hz, 1H), 2.79 (d, J=0.8 Hz, 3H).

### Example 213: 6-(3-methoxyphenyl)-5-(4-methylquinolin-6-yl)imidazo[1,2-a]pyrazin-8-amine

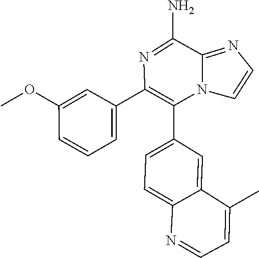

The title compound was synthesized following the approach outlined in Procedure F, substituting (4-methylquinolin-6-yl)boronic acid (example 129) for (8-fluoroquinolin-6-yl)boronic acid in step (a), substituting (3-methoxyphenyl)boronic acid for 3-pyridineboronic acid pinacol ester in step (e) to afford the title compound as a hydrochloride salt (yellow solid, 3.0 mg, 54.7%). ESI-MS: 382.00 [M+H]+. 1H NMR (400 MHz, DMSO-d6) δ 8.89 (d, J=5.7 Hz, 1H), 8.46-8.40 (m, 1H), 8.15 (dd, J=8.8, 0.7 Hz, 1H), 8.01 (dd, J=8.9, 1.8 Hz, 1H), 7.85 (dd, J=5.8, 0.9 Hz, 1H), 7.73 (d, J=1.3 Hz, 1H), 7.63 (d, J=1.3 Hz, 1H), 7.18 (t, J=8.0 Hz, 1H), 6.96-6.84 (m, 3H), 3.55 (s, 3H), 2.76 (d, J=0.9 Hz, 3H).

### Example 214: 6-(1-methyl-1H-pyrazol-3-yl)-5-(4-methylquinolin-6-yl)imidazo[1,2-a]pyrazin-8-amine

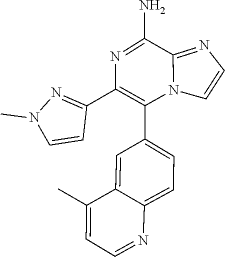 [SMILES: Cc1ccnc2ccc(-c3c(-c4ccn(C)n4)nc(N)c4nccn34)cc12]

The title compound was synthesized following the approach outlined in Procedure F, substituting (4-methylquinolin-6-yl)boronic acid (example 129) for (8-fluoroquinolin-6-yl)boronic acid in step (a), substituting 1-methyl-3-(4,4,5,5-tetramethyl-1,3,2-dioxaborolan-2-yl)-1H-pyrazole for 3-pyridineboronic acid pinacol ester in step (e) to afford the title compound as a hydrochloride salt (yellow solid, 7.0 mg, 48.8%). ESI-MS: 356.6 [M+H]+. 1H NMR (400 MHz, DMSO-d6) δ 8.96 (d, J=5.8 Hz, 1H), 8.59-8.54 (m, 1H), 8.27 (dd, J=8.8, 0.7 Hz, 1H), 8.04 (dd, J=8.9, 1.8 Hz, 1H), 7.92 (dd, J=5.7, 0.9 Hz, 1H), 7.70 (d, J=1.3 Hz, 1H), 7.52 (d, J=1.3 Hz, 1H), 7.32 (d, J=2.4 Hz, 1H), 5.65 (d, J=2.4 Hz, 1H), 3.74 (s, 3H), 2.85 (d, J=0.9 Hz, 3H).

### Example 215: 4-[8-amino-5-(4-methylquinolin-6-yl)imidazo[1,2-a]pyrazin-6-yl]-2-fluorobenzonitrile

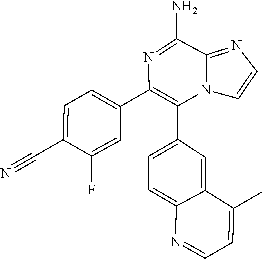 [SMILES: Cc1ccnc2ccc(-c3c(-c4ccc(C#N)c(F)c4)nc(N)c4nccn34)cc12]

The title compound was synthesized following the approach outlined in Procedure F, substituting (4-methylquinolin-6-yl)boronic acid (example 129) for (8-fluoroquinolin-6-yl)boronic acid in step (a), substituting (4-cyano-3-fluorophenyl)boronic acid for 3-pyridineboronic acid pinacol ester in step (e) to afford the title compound as a hydrochloride salt (yellow solid, 15.0 mg, 76.3%). ESI-MS: 394.95 [M+H]+. 1H NMR (400 MHz, DMSO-d6) δ 8.94 (d, J=5.8 Hz, 1H), 8.49 (d, J=1.6 Hz, 1H), 8.19 (d, J=8.8 Hz, 1H), 7.99 (dd, J=8.9, 1.8 Hz, 1H), 7.90 (dd, J=5.8, 0.9 Hz, 1H), 7.75 (d, J=1.4 Hz, 1H), 7.64 (d, J=1.4 Hz, 1H), 7.59 (dd, J=8.1, 6.5 Hz, 1H), 7.32 (dd, J=9.9, 1.6 Hz, 1H), 7.26 (dd, J=8.1, 1.6 Hz, 1H), 2.81 (d, J=0.8 Hz, 3H).

### Example 216: 6-(5-methylfuran-2-yl)-5-(4-methylquinolin-6-yl)imidazo[1,2-a]pyrazin-8-amine

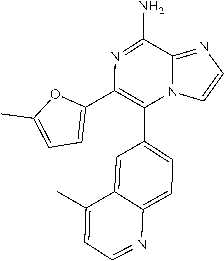 [SMILES: Cc1ccc(-c2nc(N)c3nccn3c2-c2ccc3nccc(C)c3c2)o1]

The title compound was synthesized following the approach outlined in Procedure F, substituting (4-methylquinolin-6-yl)boronic acid (example 129) for (8-fluoroquinolin-6-yl)boronic acid in step(a), substituting (5-methylfuran-2-yl)boronic acid for 3-pyridineboronic acid pinacol ester in step (e) to afford the title compound as a hydrochloride salt (brown solid, 5.0 mg, 45.35%). ESI-MS: 356.30 [M+H]+. 1H NMR (400 MHz, DMSO-d6) δ 9.13 (d, J=5.1 Hz, 1H), 8.56 (d, J=1.8 Hz, 1H), 8.40 (d, J=8.7 Hz, 1H), 8.01 (dd, J=8.7, 1.8 Hz, 1H), 7.84 (d, J=5.0 Hz, 1H), 7.80 (d, J=1.3 Hz, 1H), 7.56 (d, J=1.4 Hz, 1H), 6.12 (d, J=3.3 Hz, 1H), 6.06 (dd, J=3.3, 1.2 Hz, 1H), 2.84 (s, 3H), 2.05 (s, 3H).

### Example 217: {4-[8-amino-5-(quinolin-6-yl)imidazo[1,2-a]pyrazin-6-yl]thiophen-2-yl}methanol

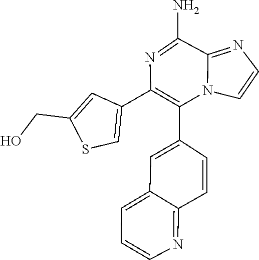 [SMILES: Nc1nc(-c2csc(CO)c2)c(-c2ccc3ncccc3c2)n2ccnc12]

The title compound was synthesized following the approach outlined in Procedure F, substituting (quinolin-6-yl)boronic acid for (8-fluoroquinolin-6-yl)boronic acid in step (a), then substituting (5-(hydroxymethyl)thiophen-2-yl)boronic acid for 3-pyridineboronic acid pinacol ester in step (e) to afford the title compound as a hydrochloride salt (brown solid, 18.8 mg, 11.4% over 2 steps). ESI-MS: 374.2 [M+H]+. 1H NMR (400 MHz, DMSO-d6) δ 9.18-9.14 (m, 1H), 8.72 (d, J=8.4 Hz, 1H), 8.37-8.26 (m, 2H), 7.93-7.78 (m, 3H), 7.64 (d, J=1.3 Hz, 1H), 7.35 (s, 1H), 6.86-6.80 (m, 1H), 4.48-4.42 (m, 2H).

### Example 218: 6-(6-fluoropyridin-2-yl)-5-(4-methylquinolin-6-yl)imidazo[1,2-a]pyrazin-8-amine

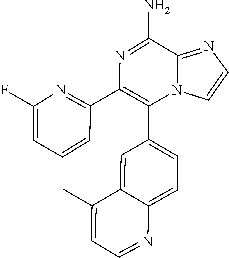 [SMILES: Cc1ccnc2ccc(-c3c(-c4cccc(F)n4)nc(N)c4nccn34)cc12]

The title compound was synthesized following the approach outlined in Procedure F, substituting (4-methylquinolin-6-yl)boronic acid (example 129) for (8-fluoroquinolin-6-yl)boronic acid in step (a), substituting (6-fluoropyridin-2-yl)boronic acid for 3-pyridineboronic acid pinacol ester step (e) to afford the title compound as a hydrochloride salt (yellow solid, 7.0 mg, 76.3%). ESI-MS: 371.30 [M+H]+. 1H NMR (400 MHz, DMSO-d6) δ 8.80 (d, J=4.4 Hz, 1H), 8.10 (d, J=1.8 Hz, 1H), 8.04 (d, J=8.6 Hz, 1H), 7.88 (q, J=8.2 Hz, 1H), 7.72 (dd, J=8.7, 1.9 Hz, 1H), 7.60-7.56 (m, 2H), 7.52 (d, J=1.2 Hz, 1H), 7.41 (dd, J=4.3, 1.1 Hz, 1H), 7.26 (s, 2H).

### Example 219: 1-{4-[8-amino-5-(quinolin-6-yl)imidazo[1,2-a]pyrazin-6-yl]pyridin-2-yl}ethan-1-one

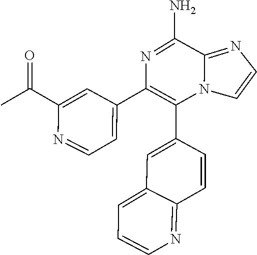 [SMILES: CC(=O)c1cc(-c2nc(N)c3nccn3c2-c2ccc3ncccc3c2)ccn1]

The title compound was synthesized following the approach outlined in Procedure F, substituting (quinolin-6-yl)boronic acid for (8-fluoroquinolin-6-yl)boronic acid in step (a), then substituting 1-(4-(4,4,5,5-tetramethyl-1,3,2-dioxaborolan-2-yl)pyridin-2-yl)ethanone-2-acetylpyridine-4-boronic acid pinacol ester for 3-pyridineboronic acid pinacol ester in step (e) to afford the title compound as a hydrochloride salt (yellow solid, 10 mg, 11.6%). ESI-MS: 382.00 [M+H]+. 1H NMR (400 MHz, DMSO-d6) δ 9.14-9.06 (m, 1H), 8.62-8.53 (m, 1H), 8.50 (d, J=5.2 Hz, 1H), 8.27-8.16 (m, 2H), 8.03 (s, 1H), 7.90-7.83 (m, 1H), 7.81-7.69 (m, 2H), 7.61 (s, 1H), 7.42-7.36 (m, 1H), 2.55 (s, 3H).

### Example 220: 5-(4-methylquinolin-6-yl)-6-(pyridin-4-yl)imidazo[1,2-a]pyrazin-8-amine

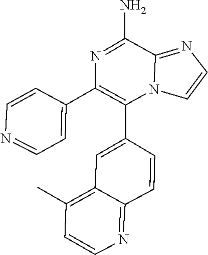 [SMILES: Cc1ccnc2ccc(-c3c(-c4ccncc4)nc(N)c4nccn34)cc12]

The title compound was synthesized following the approach outlined in Procedure F, substituting (4-methylquinolin-6-yl)boronic acid (example 129) for (8-fluoroquinolin-6-yl)boronic acid in step (a), substituting 4-pyridineboronic acid pinacol for 3-pyridineboronic acid pinacol ester in step (e) and performing this step at 100° C. for 48 h to afford the title compound as a hydrochloride salt (brown solid, 7.0 mg, 60.31%). ESI-MS: 353.30 [M+H]+. 1H NMR (400 MHz, Deuterium Oxide) δ 8.97 (d, J=5.8 Hz, 1H), 8.55 (s, 1H), 8.48 (d, J=6.2 Hz, 2H), 8.27 (d, J=8.8 Hz, 1H), 8.06 (d, J=9.1 Hz, 1H), 7.92 (d, J=5.7 Hz, 1H), 7.86 (d, J=6.2 Hz, 2H), 7.67 (s, 1H), 7.54 (s, 1H), 2.82 (s, 3H).

### Example 221: 4-[8-amino-5-(4-methylquinolin-6-yl)imidazo[1,2-a]pyrazin-6-yl]benzonitrile

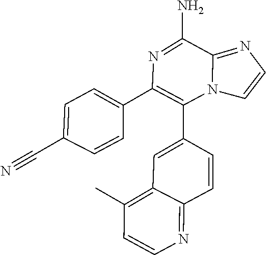 [SMILES: Cc1ccnc2ccc(-c3c(-c4ccc(C#N)cc4)nc(N)c4nccn34)cc12]

The title compound was synthesized following the approach outlined in Procedure F, substituting (4-methylquinolin-6-yl)boronic acid (example 129) for (8-fluoroquinolin-6-yl)boronic acid in step (a), substituting 4-cyanophenylboronic acid for 3-pyridineboronic acid pinacol ester in step (e) and performing this step at 115° C. for 24 h to afford the title compound as a hydrochloride salt (yellow solid, 25.0 mg, 23.5%). ESI-MS: 377.20 [M+H]+. 1H NMR (400 MHz, DMSO-d6) δ 9.07 (d, J=5.0 Hz, 1H), 8.45 (s, 1H), 8.25 (d, J=8.8 Hz, 1H), 7.90-7.86 (m, 1H), 7.83 (s, 1H), 7.79 (d, J=5.1 Hz, 1H), 7.76-7.73 (m, 2H), 7.72 (d, J=1.8 Hz, 1H), 7.54-7.50 (m, 2H), 2.74 (s, 3H)

### Example 222: 4-[8-amino-5-(8-chloroquinolin-6-yl)imidazo[1,2-a]pyrazin-6-yl]benzonitrile

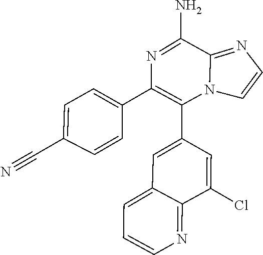 [SMILES: N#Cc1ccc(-c2nc(N)c3nccn3c2-c2cc(Cl)c3ncccc3c2)cc1]

The title compound was synthesized following the approach outlined in Procedure F, substituting (8-chloroquinolin-6-yl)boronic acid for (8-fluoroquinolin-6-yl)boronic acid in step (a), substituting 4-cyanophenylboronic acid for 3-pyridineboronic acid pinacol ester in step (e) and performing this step at 100° C. for 24 h to afford the title compound as a hydrochloride salt (yellow solid, 52.0 mg, 33.9%). ESI-MS: 397.20 [M+H]+. 1H NMR (400 MHz, DMSO-d6) δ 9.11 (dd, J=4.2, 1.7 Hz, 1H), 8.47 (dd, J=8.4, 1.7 Hz, 1H), 8.10 (d, J=1.8 Hz, 1H), 8.00 (d, J=1.8 Hz, 1H), 7.94 (d, J=1.4 Hz, 1H), 7.83 (d, J=1.4 Hz, 1H), 7.81-7.76 (m, 2H), 7.72 (dd, J=8.3, 4.3 Hz, 1H), 7.59-7.51 (m, 2H).

### Example 223: 4-[8-amino-5-(quinolin-6-yl)imidazo[1,2-a]pyrazin-6-yl]benzonitrile

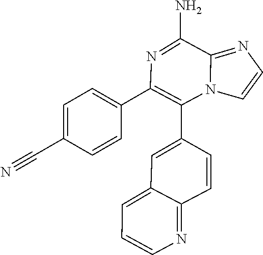 [SMILES: N#Cc1ccc(-c2nc(N)c3nccn3c2-c2ccc3ncccc3c2)cc1]

The title compound was synthesized following the approach outlined in Procedure F, substituting (quinolin-6-yl)boronic acid for (8-fluoroquinolin-6-yl)boronic acid in step (a), substituting 4-cyanophenylboronic acid for 3-pyridineboronic acid pinacol ester in step (e) and performing this step at 100° C. for 48 h to afford the title compound as a hydrochloride salt (yellow solid, 33.0 mg, 38.7%). ESI-MS: 363.20 [M+H]+. 1H NMR (400 MHz, DMSO-d6) δ 9.12-9.08 (m, 1H), 8.59 (d, J=7.7 Hz, 1H), 8.20 (d, J=9.4 Hz, 2H), 7.87 (s, 1H), 7.82 (dd, J=8.7, 1.6 Hz, 1H), 7.78-7.68 (m, 4H), 7.51 (d, J=8.3 Hz, 2H).

### Example 224: {5-[8-amino-5-(quinolin-6-yl)imidazo[1,2-a]pyrazin-6-yl]furan-2-yl}methanol

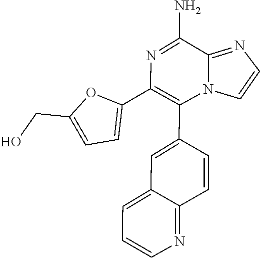 [SMILES: Nc1nc(-c2ccc(CO)o2)c(-c2ccc3ncccc3c2)n2ccnc12]

The title compound was synthesized following the approach outlined in Procedure F, substituting (quinolin-6-yl)boronic acid for (8-fluoroquinolin-6-yl)boronic acid in step (a), substituting [5-(methoxycarbonyl)furan-2-yl]boronic acid for 3-pyridineboronic acid pinacol ester in step (e) and then final product was reduced to alcohol using 1M LiAlH₄ in THE (6.0 equiv.) and reaction was performed from 0° to 25° C. for 1 h, crude product was extracted using DCM and water, purified by flash column chromatography (using gradient of MeOH from 0 to 5% in DCM) to afford the title compound as a hydrochloride salt (light yellow solid, 2.0 mg, 8.6% over 2 steps). ESI-MS: 358.20 [M+H]+. 1H NMR (400 MHz, DMSO-d6) δ 9.13 (dd, J=4.5, 1.7 Hz, 1H), 8.66-8.61 (m, 1H), 8.32-8.27 (m, 2H), 7.88 (dd, J=8.7, 2.0 Hz, 1H), 7.79-7.74 (m, 2H), 7.50 (s, 1H), 6.21 (d, J=3.4 Hz, 1H), 5.92 (d, J=3.4 Hz, 1H), 4.20 (s, 2H).

### Example 225: 4-[8-amino-5-(quinolin-6-yl)imidazo[1,2-a]pyrazin-6-yl]pyridine-2-carbonitrile

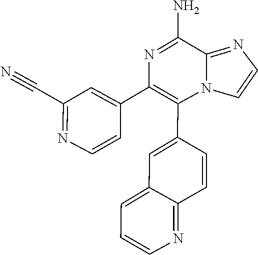 [SMILES: N#Cc1cc(-c2nc(N)c3nccn3c2-c2ccc3ncccc3c2)ccn1]

The title compound was synthesized following the approach outlined in Procedure F, substituting (quinolin-6-yl)boronic acid for (8-fluoroquinolin-6-yl)boronic acid in step (a), substituting (2-cyanopyridin-4-yl)boronic acid for 3-pyridineboronic acid pinacol ester in step (e) to afford the title compound as a light yellow solid (7.0 mg, 7%). ESI-MS: 364.20 [M+H]+. 1H NMR (400 MHz, DMSO-d6) δ 9.01 (dd, J=4.2, 1.7 Hz, 1H), 8.49 (dd, J=5.2, 0.8 Hz, 1H), 8.37 (dd, J=8.6, 1.9 Hz, 1H), 8.18-8.12 (m, 2H), 7.94 (dd, J=1.8, 0.8 Hz, 1H), 7.80 (dd, J=8.7, 2.0 Hz, 1H), 7.64-7.59 (m, 2H), 7.51 (d, J=1.2 Hz, 1H), 7.41 (dd, J=5.2, 1.7 Hz, 1H), 7.40 (s, 2H).

### Example 226: 5-(quinolin-6-yl)-6-(1,3-thiazol-4-yl)imidazo[1,2-a]pyrazin-8-amine

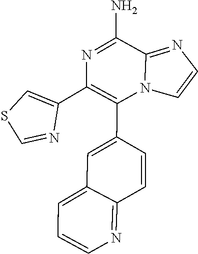 [SMILES: Nc1nc(-c2cscn2)c(-c2ccc3ncccc3c2)n2ccnc12]

The title compound was synthesized following the approach outlined in Procedure F, substituting (quinolin-6-yl)boronic acid for (8-fluoroquinolin-6-yl)boronic acid in step (a), substituting 4-(tributylstannyl)-1,3-thiazole (1.5 equiv.) for 3-pyridineboronic acid pinacol ester in step (e) performing this reaction for 21 h at 90° C. without base, then for 23 h with additional amount of catalyst Pd(PPh₃)₄ (0.05 equiv.) and 4-(tributylstannyl)-1,3-thiazole (1.5 equiv.) at 100° C. to afford the title compound as a hydrochloride salt (light yellow solid, 15.0 mg, 14.9%). ESI-MS: 345.2 [M+H]+. 1H NMR (400 MHz, DMSO-d6) δ 9.20-9.15 (m, 2H), 8.73-8.67 (m, 1H), 8.38-8.33 (m, 2H), 7.91 (dd, J=8.7, 2.0 Hz, 1H), 7.85 (d, J=1.3 Hz, 1H), 7.83-7.79 (m, 1H), 7.61 (d, J=1.3 Hz, 1H), 7.15 (s, 1H).

### Example 227: 6-(3-aminophenyl)-5-(quinolin-6-yl)imidazo[1,2-a]pyrazin-8-amine

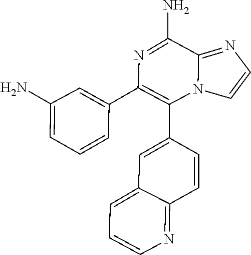 [SMILES: Nc1cccc(-c2nc(N)c3nccn3c2-c2ccc3ncccc3c2)c1]

The title compound was synthesized following the approach outlined in Procedure F, substituting (quinolin-6-yl)boronic acid for (8-fluoroquinolin-6-yl)boronic acid in step (a), (3-aminophenyl)boronic acid for 3-pyridineboronic acid pinacol ester in step (e) performing this reaction at 90° C. overnight, then for next 16 h with additional amount of catalyst Pd(PPh₃)₄ (0.05 equiv.) at 100° C. to afford the title compound as a hydrochloride salt (orange solid, 27.0 mg, 32.6%). ESI-MS: 353.3 [M+H]+. 1H NMR (400 MHz, DMSO-d6) δ 9.15 (d, J=3.6 Hz, 1H), 8.73 (d, J=7.6 Hz, 1H), 8.34-8.21 (m, 2H), 7.93 (s, 1H), 7.89-7.78 (m, 2H), 7.76-7.71 (m, 1H), 7.32-7.20 (m, 2H), 7.20-7.06 (m, 2H).

### Example 228:2-{4-[8-amino-5-(quinolin-6-yl)imidazo[1,2-a]pyrazin-6-yl]-1H-pyrazol-1-yl}ethan-1-ol

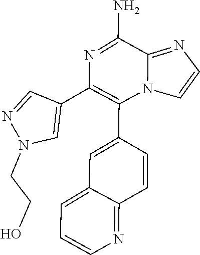 [SMILES: Nc1nc(-c2cnn(CCO)c2)c(-c2ccc3ncccc3c2)n2ccnc12]

The title compound was synthesized following the approach outlined in Procedure F, substituting (quinolin-6-yl)boronic acid for (8-fluoroquinolin-6-yl)boronic acid in step (a), [1-(2-hydroxyethyl)-1H-pyrazol-4-yl]boronic acid for 3-pyridineboronic acid pinacol ester in step (e) to afford the title compound as a hydrochloride salt (yellow solid, 28.0 mg, 26%). ESI-MS: 372.2 [M+H]+. 1H NMR (400 MHz, DMSO-d6) δ 9.26 (d, J=4.7 Hz, 1H), 8.93-8.85 (m, 1H), 8.49-8.40 (m, 2H), 8.03-7.85 (m, 3H), 7.66 (s, 1H), 7.59 (d, J=1.3 Hz, 1H), 7.18 (s, 1H), 4.01 (t, J=5.5 Hz, 2H), 3.57 (t, J=5.5 Hz, 2H).

### Example 229: 5-[8-amino-5-(quinolin-6-yl)imidazo[1,2-a]pyrazin-6-yl]pyridine-3-carbonitrile

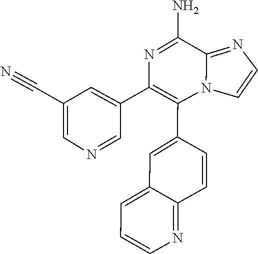 [SMILES: N#Cc1cncc(-c2nc(N)c3nccn3c2-c2ccc3ncccc3c2)c1]

The title compound was synthesized following the approach outlined in Procedure F, substituting (quinolin-6-yl)boronic acid for (8-fluoroquinolin-6-yl)boronic acid in step (a), (5-cyanopyridin-3-yl)boronic acid for 3-pyridineboronic acid pinacol ester in step (e) performing this step under microwave irradiation at 120° C. with DMF as a solvent instead of 1,4-dioxane to afford the title compound (yellow solid, 28.0 mg, 26%). ESI-MS: 364.2 [M+H]+. 1H NMR (400 MHz, DMSO-d6) δ 8.99 (dd, J=4.2, 1.8 Hz, 1H), 8.80 (d, J=2.0 Hz, 1H), 8.57 (d, J=2.1 Hz, 1H), 8.38-8.34 (m, 1H), 8.24 (t, J=2.1 Hz, 1H), 8.14-8.09 (m, 2H), 7.79 (dd, J=8.6, 2.0 Hz, 1H), 7.62-7.58 (m, 2H), 7.53 (d, J=1.2 Hz, 1H), 7.37 (s, 2H).

### Example 230: 5-[8-amino-5-(quinolin-6-yl)imidazo[1,2-a]pyrazin-6-yl]thiophene-2-carbonitrile

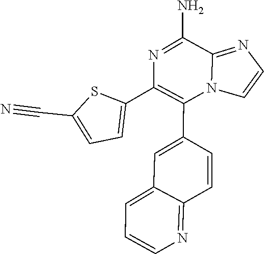 [SMILES: N#Cc1ccc(-c2nc(N)c3nccn3c2-c2ccc3ncccc3c2)s1]

The title compound was synthesized following the approach outlined in Procedure F, substituting (quinolin-6-yl)boronic acid for (8-fluoroquinolin-6-yl)boronic acid in step (a), (5-cyanothiophen-2-yl)boronic acid for 3-pyridineboronic acid pinacol ester in step (e) performing this step with different base KF (5.0 equiv.) and using palladium (II) acetate (0.07 equiv.) and Xphos (0. 1 equiv) using MW irradiation at 130° C. for 2 h to afford the title compound (yellow solid, 4.0 mg, 3.6%). ESI-MS: 369.2 [M+H]+. 1H NMR (400 MHz, DMSO-d6) δ 9.07 (dd, J=4.2, 1.7 Hz, 1H), 8.49-8.45 (m, 1H), 8.32-8.27 (m, 2H), 7.87 (dd, J=8.7, 1.9 Hz, 1H), 7.67 (dd, J=8.3, 4.2 Hz, 1H), 7.55 (d, J=4.1 Hz, 1H), 7.52 (d, J=1.2 Hz, 1H), 7.39 (s, 2H), 7.26 (d, J=1.2 Hz, 1H), 6.13 (d, J=4.2 Hz, 1H).

### Example 231: 6-(2-methylpyridin-4-yl)-5-(quinolin-6-yl)imidazo[1,2-a]pyrazin-8-amine

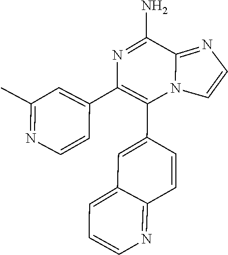 [SMILES: Cc1cc(-c2nc(N)c3nccn3c2-c2ccc3ncccc3c2)ccn1]

The title compound was synthesized following the approach outlined in Procedure F, substituting (quinolin-6-yl)boronic acid for (8-fluoroquinolin-6-yl)boronic acid in step (a), (2-methylpyridin-4-yl)boronic acid for 3-pyridineboronic acid pinacol ester in step (e) performing this step at 100° C. using Pd(dppf)Cl₂ (0.02 equiv.) as a catalyst to afford the title compound as a hydrochloride salt (yellow solid, 16.0 mg, 43%). ESI-MS: 353.2 [M+H]+. 1H NMR (400 MHz, DMSO-d6) δ 9.18 (dd, J=4.6, 1.7 Hz, 1H), 8.69 (d, J=8.3 Hz, 1H), 8.46 (d, J=6.2 Hz, 1H), 8.38-8.28 (m, 2H), 7.98-7.89 (m, 2H), 7.86-7.78 (m, 2H), 7.67 (d, J=1.5 Hz, 1H), 7.38 (dd, J=6.2, 1.8 Hz, 1H), 2.62 (s, 3H).

### Example 232: 5-[8-amino-5-(quinolin-6-yl)imidazo[1,2-a]pyrazin-6-yl]pyridin-2-amine

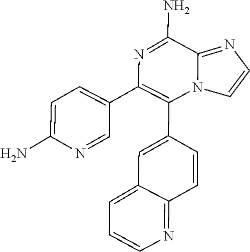 [SMILES: Nc1ccc(-c2nc(N)c3nccn3c2-c2ccc3ncccc3c2)cn1]

The title compound was synthesized following the approach outlined in Procedure F, substituting (quinolin-6-yl)boronic acid for (8-fluoroquinolin-6-yl)boronic acid in step (a), (6-aminopyridin-3-yl)boronic acid for 3-pyridineboronic acid pinacol ester in step (e) performing this step at 100° C. for 24 h to afford the title compound as a hydrochloride salt (brown solid, 13.0 mg, 26%). ESI-MS: 354.2 [M+H]+. 1H NMR (400 MHz, DMSO-d6) δ 9.16 (dd, J=4.6, 1.6 Hz, 1H), 8.73 (d, J=8.4 Hz, 1H), 8.37-8.27 (m, 3H), 7.92 (d, J=1.7 Hz, 1H), 7.88 (dd, J=8.6, 2.1 Hz, 1H), 7.83 (dd, J=8.3, 4.6 Hz, 1H), 7.75 (d, J=1.5 Hz, 1H), 7.70 (dd, J=9.3, 2.2 Hz, 1H), 6.85 (d, J=9.3 Hz, 1H).

### Example 233: 6-(2-methoxypyridin-4-yl)-5-(quinolin-6-yl)imidazo[1,2-a]pyrazin-8-amine

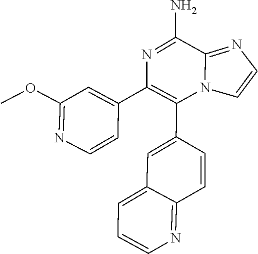 [SMILES: COc1cc(-c2nc(N)c3nccn3c2-c2ccc3ncccc3c2)ccn1]

The title compound was synthesized following the approach outlined in Procedure F, substituting (quinolin-6-yl)boronic acid for (8-fluoroquinolin-6-yl)boronic acid in step (a), (6-aminopyridin-3-yl)boronic acid for 3-pyridineboronic acid pinacol ester in step (e) performing this step at 100° C. for 24 h to afford the title compound as a white solid (17.0 mg, 16%). ESI-MS: 369.2 [M+H]+. 1H NMR (400 MHz, DMSO-d6) δ 8.99 (dd, J=4.2, 1.7 Hz, 1H), 8.37 (dd, J=8.3, 1.7 Hz, 1H), 8.14-8.09 (m, 2H), 7.92 (d, J=5.4 Hz, 1H), 7.77 (dd, J=8.6, 2.0 Hz, 1H), 7.60 (dd, J=8.3, 4.2 Hz, 1H), 7.56 (d, J=1.2 Hz, 1H), 7.43 (d, J=1.2 Hz, 1H), 7.27 (s, 2H), 6.82 (dd, J=5.4, 1.5 Hz, 1H), 6.73-6.68 (m, 1H), 3.71 (s, 3H).

### Example 234: 6-(3-methoxyphenyl)-5-(quinolin-6-yl)imidazo[1,2-a]pyrazin-8-amine

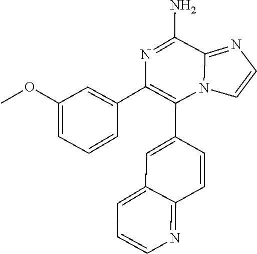 [SMILES: COc1cccc(-c2nc(N)c3nccn3c2-c2ccc3ncccc3c2)c1]

The title compound was synthesized following the approach outlined in Procedure F, substituting (quinolin-6-yl)boronic acid for (8-fluoroquinolin-6-yl)boronic acid in step (a), (3-methoxyphenyl)boronic acid for 3-pyridineboronic acid pinacol ester in step (e) performing this step at 100° C. to afford the title compound as a hydrochloride salt (yellow solid, 45.0 mg, 39.5%). ESI-MS: 368.2 [M+H]+. 1H NMR (400 MHz, DMSO-d6) δ 9.16 (dd, J=4.6, 1.6 Hz, 1H), 8.74 (d, J=8.4 Hz, 1H), 8.35-8.23 (m, 2H), 7.94-7.85 (m, 2H), 7.87-7.78 (m, 1H), 7.74 (d, J=1.3 Hz, 1H), 7.18 (t, J=8.0 Hz, 1H), 7.01 (t, J=2.1 Hz, 1H), 6.88 (ddd, J=8.7, 3.3, 1.8 Hz, 2H), 3.62 (s, 3H).

### Example 235: 6-(3-nitrophenyl)-5-(quinolin-6-yl)imidazo[1,2-a]pyrazin-8-amine

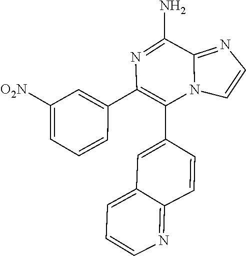 [SMILES: Nc1nc(-c2cccc([N+](=O)[O-])c2)c(-c2ccc3ncccc3c2)n2ccnc12]

The title compound was synthesized following the approach outlined in Procedure F, substituting (quinolin-6-yl)boronic acid for (8-fluoroquinolin-6-yl)boronic acid in step (a), 3-nitrophenylboronic acid for 3-pyridineboronic acid pinacol ester in step (e) performing this step at 100° C. to afford the title compound as a hydrochloride salt (yellow solid, 5.0 mg, 35.6%). ESI-MS: 383.2 [M+H]+. 1H NMR (400 MHz, DMSO-d6) δ 9.07 (dd, J=4.5, 1.7 Hz, 1H), 8.54 (d, J=8.4 Hz, 1H), 8.30 (t, J=2.0 Hz, 1H), 8.22 (d, J=1.9 Hz, 1H), 8.17 (d, J=8.8 Hz, 1H), 8.10 (ddd, J=8.2, 2.4, 1.0 Hz, 1H), 7.85 (dd, J=8.7, 2.0 Hz, 1H), 7.81 (d, J=1.4 Hz, 1H), 7.71 (dd, J=8.3, 4.4 Hz, 1H), 7.67 (d, J=1.4 Hz, 1H), 7.64 (dt, J=7.9, 1.3 Hz, 1H), 7.47 (t, J=8.0 Hz, 1H).

### Example 236: 6-(6-methoxypyridin-3-yl)-5-(quinolin-6-yl)imidazo[1,2-a]pyrazin-8-amine

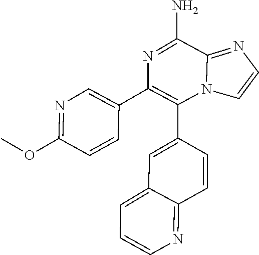 [SMILES: COc1ccc(-c2nc(N)c3nccn3c2-c2ccc3ncccc3c2)cn1]

The title compound was synthesized following the approach outlined in Procedure F, substituting (quinolin-6-yl)boronic acid for (8-fluoroquinolin-6-yl)boronic acid in step (a), 6-metoxypiridine-3-boronic acid for 3-pyridineboronic acid pinacol ester in step (e) performing this step at 100° C. to afford the title compound as a yellow solid (15.0 mg, 13.8%). ESI-MS: 369.2 [M+H]+. 1H NMR (400 MHz, DMSO-d6) δ 8.98 (dd, J=4.3, 1.7 Hz, 1H), 8.37 (dd, J=8.4, 2.0 Hz, 1H), 8.12 (d, J=2.0 Hz, 1H), 8.09 (d, J=8.7 Hz, 1H), 8.00 (dd, J=2.5, 0.7 Hz, 1H), 7.73 (dd, J=8.7, 2.0 Hz, 1H), 7.65 (dd, J=8.6, 2.5 Hz, 1H), 7.59 (dd, J=8.3, 4.2 Hz, 1H), 7.54 (d, J=1.2 Hz, 1H), 7.44 (d, J=1.2 Hz, 1H), 7.20 (s, 2H), 6.67 (dd, J=8.6, 0.7 Hz, 1H), 3.73 (s, 3H).

### Example 237: Methyl 5-[8-amino-5-(quinolin-6-yl)imidazo[1,2-a]pyrazin-6-yl]furan-2-carboxylate

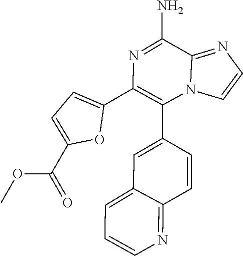 [SMILES: COC(=O)c1ccc(-c2nc(N)c3nccn3c2-c2ccc3ncccc3c2)o1]

The title compound was synthesized following the approach outlined in Procedure F, substituting (quinolin-6-yl)boronic acid for (8-fluoroquinolin-6-yl)boronic acid in step (a), (5-(methoxycarbonyl)furan-2-yl)boronic acid for 3-pyridineboronic acid pinacol ester in step (e) performing this step with KF (4.0 equiv.), palladium (II) acetate (0.06 equiv.) and Xphos (0.10 equiv) using MW irradiation at 130° C. for 1.5 h to afford the title compound as a hydrochloride salt (yellow solid, 10.0 mg, 5.9%). ESI-MS: 386.2 [M+H]+. 1H NMR (400 MHz, DMSO-d6) δ 9.24-9.18 (m, 1H), 8.82-8.75 (m, 1H), 8.42-8.32 (m, 2H), 7.98 (dd, J=8.7, 1.7 Hz, 1H), 7.87 (dd, J=8.2, 4.6 Hz, 1H), 7.80-7.75 (m, 1H), 7.51 (d, J=1.3 Hz, 1H), 7.19 (d, J=3.7 Hz, 1H), 6.42 (d, J=3.7 Hz, 1H), 3.52 (s, 3H).

### Example 238: 5-[8-amino-5-(quinolin-6-yl)imidazo[1,2-a]pyrazin-6-yl]-3-methylpyridine-2-carbonitrile

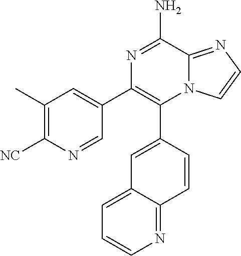 [SMILES: Cc1cc(-c2nc(N)c3nccn3c2-c2ccc3ncccc3c2)cnc1C#N]

The title compound was synthesized following the approach outlined in Procedure F, substituting (quinolin-6-yl)boronic acid for (8-fluoroquinolin-6-yl)boronic acid in step (a), (6-cyano-5-methylpyridin-3-yl)boronic acid for 3-pyridineboronic acid pinacol in step (e) to afford the title compound as a hydrochloride salt (yellow solid, 16.0 mg, 31%). ESI-MS: 378.2 [M+H]+. 1H NMR (400 MHz, DMSO-d6) δ 9.13 (dd, J=4.5, 1.4 Hz, 1H), 8.64 (d, J=8.4 Hz, 1H), 8.30-8.19 (m, 3H), 8.02-7.98 (m, 1H), 7.88 (dd, J=8.7, 1.9 Hz, 1H), 7.85-7.81 (m, 1H), 7.79 (dd, J=8.3, 4.6 Hz, 1H), 7.67 (d, J=1.2 Hz, 1H), 2.40 (s, 3H).

### Example 239: 3-[8-amino-5-(quinolin-6-yl)imidazo[1,2-a]pyrazin-6-yl]phenol

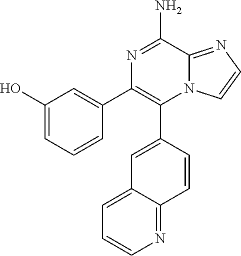 [SMILES: Nc1nc(-c2cccc(O)c2)c(-c2ccc3ncccc3c2)n2ccnc12]

The title compound was synthesized following the approach outlined in Procedure F, substituting (quinolin-6-yl)boronic acid for (8-fluoroquinolin-6-yl)boronic acid in step (a), (3-hydroxy)phenylboronic acid for 3-pyridineboronic acid pinacol in step (e) performing this reaction at 100° C. to afford the title compound as a yellow solid (22.0 mg, 21%). ESI-MS: 354.0 [M+H]+. 1H NMR (400 MHz, DMSO-d6) δ 9.22 (s, 1H), 8.95 (dd, J=4.2, 1.7 Hz, 1H), 8.35 (dd, J=8.2, 1.7 Hz, 1H), 8.07-8.02 (m, 2H), 7.69 (dd, J=8.6, 2.0 Hz, 1H), 7.57 (dd, J=8.3, 4.2 Hz, 1H), 7.53 (d, J=1.2 Hz, 1H), 7.43 (d, J=1.1 Hz, 1H), 7.12 (s, 2H), 6.89 (t, J=7.9 Hz, 1H), 6.81 (dd, J=2.5, 1.6 Hz, 1H), 6.63 (dt, J=7.7, 1.3 Hz, 1H), 6.53 (ddd, J=8.0, 2.5, 1.0 Hz, 1H).

### Example 240: 5-(8-fluoroquinolin-6-yl)-6-(furan-2-yl)imidazo[1,2-a]pyrazin-8-amine

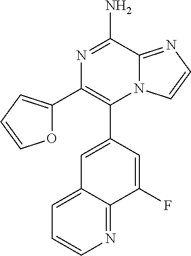 [SMILES: Nc1nc(-c2ccco2)c(-c2cc(F)c3ncccc3c2)n2ccnc12]

The title compound was synthesized following the approach outlined in Procedure F, substituting (furan-2-yl)boronic acid for 3-pyridineboronic acid pinacol in step (e) performing this reaction for 10 h to afford the title compound as a hydrochloride salt (orange solid, 25.0 mg, 65%). ESI-MS: 346.2 [M+H]+. 1H NMR (400 MHz, DMSO-d6) δ 9.10 (dd, J=4.2, 1.6 Hz, 1H), 8.58-8.53 (m, 1H), 8.06 (d, J=1.6 Hz, 1H), 7.82 (d, J=1.3 Hz, 1H), 7.78-7.72 (m, 2H), 7.65-7.60 (m, 2H), 6.46 (dd, J=3.5, 1.8 Hz, 1H), 6.37 (dd, J=3.5, 0.8 Hz, 1H).

### Example 241: 6-(4-methoxyphenyl)-5-(quinolin-6-yl)imidazo[1,2-a]pyrazin-8-amine

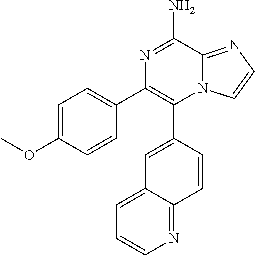 [SMILES: COc1ccc(-c2nc(N)c3nccn3c2-c2ccc3ncccc3c2)cc1]

The title compound was synthesized following the approach outlined in Procedure F, substituting (quinolin-6-yl)boronic acid for (8-fluoroquinolin-6-yl)boronic acid in step (a), (4-methoxy)phenylboronic acid for 3-pyridineboronic acid pinacol in step (e) to afford the title compound as a hydrochloride salt (yellow solid, 24.0 mg, 25%). ESI-MS: 368.2 [M+H]+. 1H NMR (400 MHz, DMSO-d6) δ 9.59-8.85 (m, 3H), 8.68 (d, J=8.4 Hz, 1H), 8.31-8.20 (m, 2H), 7.89 (s, 1H), 7.86-7.76 (m, 2H), 7.73 (d, J=1.1 Hz, 1H), 7.30 (d, J=8.8 Hz, 2H), 6.86 (d, J=8.8 Hz, 2H), 3.69 (s, 3H).

### Example 242: 6-(6-fluoropyridin-2-yl)-5-(quinolin-6-yl)imidazo[1,2-a]pyrazin-8-amine

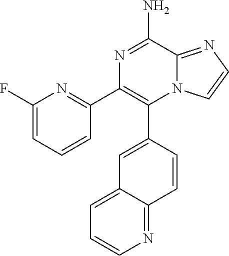 [SMILES: Nc1nc(-c2cccc(F)n2)c(-c2ccc3ncccc3c2)n2ccnc12]

The title compound was synthesized following the approach outlined in Procedure F, substituting (quinolin-6-yl)boronic acid for (8-fluoroquinolin-6-yl)boronic acid in step (a), (6-fluoropyridin-2-yl)boronic acid for 3-pyridineboronic acid pinacol in step (e) performing this reaction at 100° C. for 48 h to afford the title compound as a yellow solid (10.0 mg, 11%). ESI-MS: 357.0 [M+H]+. 1H NMR (400 MHz, DMSO-d6) δ 8.96 (dd, J=4.2, 1.7 Hz, 1H), 8.35 (dd, J=8.5, 2.0 Hz, 1H), 8.07 (d, J=8.7 Hz, 1H), 8.03 (d, J=1.9 Hz, 1H), 7.89 (m, 1H), 7.71 (dd, J=8.7, 2.0 Hz, 1H), 7.63 (dd, J=7.4, 2.5 Hz, 1H), 7.59-7.52 (m, 2H), 7.43 (d, J=1.2 Hz, 1H), 7.26 (s, 2H), 6.93 (dd, J=8.1, 2.8 Hz, 1H).

### Example 243: 6-(pyridin-4-yl)-5-(quinolin-6-yl)imidazo[1,2-a]pyrazin-8-amine

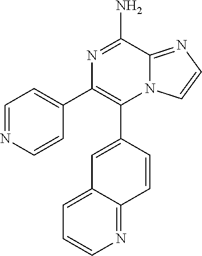 [SMILES: Nc1nc(-c2ccncc2)c(-c2ccc3ncccc3c2)n2ccnc12]

The title compound was synthesized following the approach outlined in Procedure F, substituting (quinolin-6-yl)boronic acid for (8-fluoroquinolin-6-yl)boronic acid in step (a), (pyridin-4-yl)boronic acid for 3-pyridineboronic acid pinacol in step (e) performing this reaction at 100° C. for 24 h to afford the title compound as a yellow solid (8.0 mg, 9%). ESI-MS: 339.2 [M+H]+. 1H NMR (400 MHz, DMSO-d6) δ 8.99 (dd, J=4.1, 1.7 Hz, 1H), 8.45-8.29 (m, 3H), 8.15-8.03 (m, 2H), 7.77 (dd, J=8.6, 2.0 Hz, 1H), 7.65-7.53 (m, 2H), 7.46 (s, 1H), 7.37-7.17 (m, 4H).

### Example 244:5-(8-fluoroquinolin-6-yl)-6-(6-methoxypyridin-3-yl)imidazo[1,2-a]pyrazin-8-amine

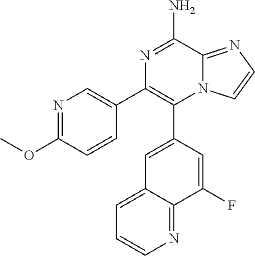 [SMILES: COc1ccc(-c2nc(N)c3nccn3c2-c2cc(F)c3ncccc3c2)cn1]

The title compound was synthesized following the approach outlined in Procedure F, substituting (6-methoxypyridin-3-yl)boronic acid for 3-pyridineboronic acid pinacol in step (e) performing this reaction at 100° C. to afford the title compound as a yellow solid (16.0 mg, 41%). ESI-MS: 387.2 [M+H]+. 1H NMR (400 MHz, DMSO-d6) δ 9.02 (dd, J=4.2, 1.4 Hz, 1H), 8.45-8.41 (m, 1H), 8.04 (d, J=2.4 Hz, 1H), 7.95-7.92 (m, 1H), 7.72-7.63 (m, 3H), 7.56-7.52 (m, 2H), 7.24 (s, 2H), 6.68 (d, J=8.6 Hz, 1H), 3.74 (s, 3H).

### Example 245: 6-(6-fluoropyridin-3-yl)-5-(8-fluoroquinolin-6-yl)imidazo[1,2-a]pyrazin-8-amine

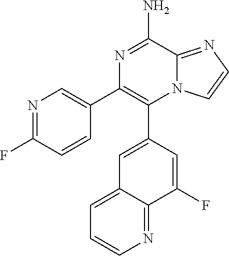 [SMILES: Nc1nc(-c2ccc(F)nc2)c(-c2cc(F)c3ncccc3c2)n2ccnc12]

The title compound was synthesized following the approach outlined in Procedure F, substituting 2-fluoropyridine-5-boronic acid for 3-pyridineboronic acid pinacol in step (e) to afford the title compound as a yellow solid (23.0 mg, 72.1%). ESI-MS: 375.2 [M+H]+. 1H NMR (400 MHz, DMSO-d6) δ 9.03 (dd, J=4.2, 1.6 Hz, 1H), 8.43-8.39 (m, 1H), 8.14-8.11 (m, 1H), 7.93-7.90 (m, 1H), 7.87 (td, J=8.3, 2.5 Hz, 1H), 7.73 (dd, J=11.4, 1.8 Hz, 1H), 7.68 (dd, J=8.4, 4.2 Hz, 1H), 7.57 (s, 2H), 7.33 (s, 2H), 7.05 (dd, J=8.5, 2.8 Hz, 1H).

### Example 246: 6-(3,4-difluorophenyl)-5-(8-fluoroquinolin-6-yl)imidazo[1,2-a]pyrazin-8-amine

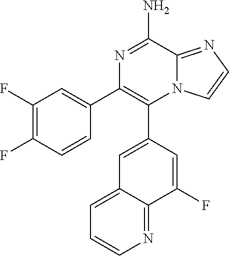 [SMILES: Nc1nc(-c2ccc(F)c(F)c2)c(-c2cc(F)c3ncccc3c2)n2ccnc12]

The title compound was synthesized following the approach outlined in Procedure F, substituting (3,4-difluorophenyl)boronic acid for 3-pyridineboronic acid pinacol in step (e) to afford the title compound as a hydrochloride salt (yellow solid, 28.0 mg, 73%). ESI-MS: 392.2 [M+H]+. 1H NMR (400 MHz, DMSO-d6) δ 9.05 (dd, J=4.2, 1.6 Hz, 1H), 8.87 (s, 2H), 8.47 (m, 1H), 7.94 (dd, J=10.8, 1.6 Hz, 2H), 7.84 (d, J=1.4 Hz, 1H), 7.76-7.67 (m, 2H), 7.53 (ddd, J=11.5, 7.8, 2.2 Hz, 1H), 7.36 (m, 1H), 7.20-7.11 (m, 1H).

### Example 247:5-(8-fluoroquinolin-6-yl)-6-[4-(trifluoromethyl)phenyl]imidazo[1,2-a]pyrazin-8-amine

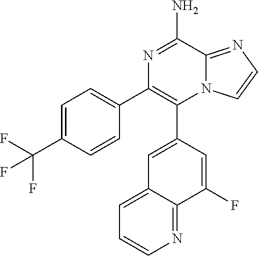 [SMILES: Nc1nc(-c2ccc(C(F)(F)F)cc2)c(-c2cc(F)c3ncccc3c2)n2ccnc12]

The title compound was synthesized following the approach outlined in Procedure F, substituting 4-(trifluoromethyl)phenylboronic acid for 3-pyridineboronic acid pinacol in step (e) to afford the title compound as a hydrochloride salt (yellow solid, 35.0 mg, 82%). ESI-MS: 424.2 [M+H]+. 1H NMR (400 MHz, DMSO-d6) δ 9.05 (dd, J=4.2, 1.6 Hz, 1H), 8.81 (s, 2H), 8.45 (m, 1H), 7.97-7.89 (m, 2H), 7.83 (d, J=1.7 Hz, 1H), 7.75-7.65 (m, 4H), 7.60 (m, 2H).

### Example 248: 6-(furan-2-yl)-5-(quinolin-6-yl)imidazo[1,2-a]pyrazin-8-amine

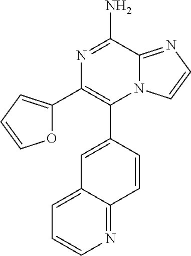 [SMILES: Nc1nc(-c2ccco2)c(-c2ccc3ncccc3c2)n2ccnc12]

The title compound was synthesized following the approach outlined in Procedure F, substituting (furan-2-yl)boronic acid for 3-pyridineboronic acid pinacol in step (e) to afford the title compound as a yellow solid (4.0 mg, 4%). ESI-MS: 328.1 [M+H]+. 1H NMR (400 MHz, DMSO-d6) δ 9.01 (dd, J=4.2, 1.8 Hz, 1H), 8.44 (dd, J=8.4, 1.7 Hz, 1H), 8.20-8.13 (m, 2H), 7.78 (dd, J=8.7, 1.8 Hz, 1H), 7.62 (dd, J=8.3, 4.2 Hz, 1H), 7.50 (d, J=1.2 Hz, 1H), 7.41 (dd, J=1.8, 0.9 Hz, 1H), 7.27 (d, J=1.2 Hz, 1H), 7.18 (s, 2H), 6.36 (dd, J=3.4, 1.8 Hz, 1H), 6.23 (dd, J=3.4, 0.9 Hz, 1H).

### Example 249: 6-(5-methylfuran-2-yl)-5-(quinolin-6-yl)imidazo[1,2-a]pyrazin-8-amine

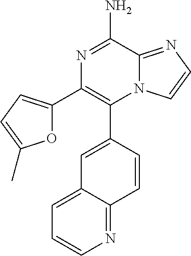 [SMILES: Cc1ccc(-c2nc(N)c3nccn3c2-c2ccc3ncccc3c2)o1]

The title compound was synthesized following the approach outlined in Procedure F, substituting (quinolin-6-yl)boronic acid for (8-fluoroquinolin-6-yl)boronic acid in step (a), 5-methylfuran-2-boronic acid pinacol ester for 3-pyridineboronic acid pinacol ester in step (e) performing this step with Pd(dppf)Cl₂ (0.05 equiv) to afford the title compound as a yellow solid (4.0 mg, 4%). ESI-MS: 342.2 [M+H]+. 1H NMR (400 MHz, DMSO-d6) δ 9.01 (dd, J=4.3, 1.7 Hz, 1H), 8.44 (dt, J=8.6, 0.9 Hz, 1H), 8.20-8.14 (m, 2H), 7.77 (dd, J=8.6, 2.0 Hz, 1H), 7.62 (dd, J=8.3, 4.2 Hz, 1H), 7.49 (d, J=1.2 Hz, 1H), 7.28 (d, J=1.2 Hz, 1H), 7.17 (s, 2H), 5.93 (d, J=1.5 Hz, 2H), 1.97 (d, J=0.7 Hz, 3H).

### Example 250: 6-(pyridin-3-yl)-5-(quinolin-6-yl)imidazo[1,2-a]pyrazin-8-amine

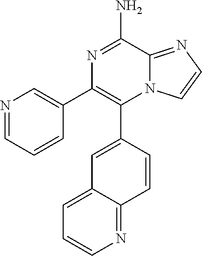 [SMILES: Nc1nc(-c2cccnc2)c(-c2ccc3ncccc3c2)n2ccnc12]

The title compound was synthesized following the approach outlined in Procedure F, substituting (quinolin-6-yl)boronic acid for (8-fluoroquinolin-6-yl)boronic acid in step (a), step (e) was performed with Pd(dppf)Cl₂ (0.05 equiv) catalyst to afford the title compound as an off-white solid (45.0 mg, 45.2%). ESI-MS: 339.2 [M+H]+. 1H NMR (400 MHz, DMSO-d6) δ 9.01 (dd, J=4.3, 1.7 Hz, 1H), 8.51 (d, J=2.1 Hz, 1H), 8.44 (dd, J=5.2, 1.6 Hz, 1H), 8.42-8.37 (m, 1H), 8.14 (d, J=2.0 Hz, 1H), 8.11 (d, J=8.7 Hz, 1H), 7.85 (d, J=7.8 Hz, 1H), 7.78 (dd, J=8.7, 2.0 Hz, 1H), 7.66 (d, J=1.2 Hz, 1H), 7.62 (dd, J=8.3, 4.3 Hz, 1H), 7.57 (d, J=1.2 Hz, 1H), 7.39 (dd, J=8.0, 5.0 Hz, 1H).

### Example 251: 6-(1-methyl-1H-pyrazol-3-yl)-5-(quinolin-6-yl)imidazo[1,2-a]pyrazin-8-amine

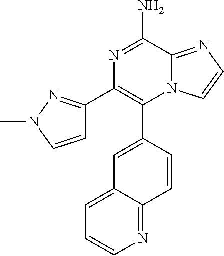 [SMILES: Cn1ccc(-c2nc(N)c3nccn3c2-c2ccc3ncccc3c2)n1]

The title compound was synthesized following the approach outlined in Procedure F, substituting (quinolin-6-yl)boronic acid for (8-fluoroquinolin-6-yl)boronic acid in step (a), substituting 1-methyl-3-(tetramethyl-1,3,2-dioxaborolan-2-yl)-1H-pyrazole for 3-pyridineboronic acid pinacol ester in step (e) to afford the title compound as a hydrochloride salt (yellow solid, 35.0 mg, 43.6%). ESI-MS: 342.2 [M+H]+. 1H NMR (400 MHz, DMSO-d6) δ 10.03 (s, 2H), 9.29-9.22 (m, 1H), 8.85 (d, J=8.4 Hz, 1H), 8.48 (d, J=8.7 Hz, 1H), 8.45-8.40 (m, 1H), 8.00 (dd, J=8.8, 1.7 Hz, 1H), 7.93-7.88 (m, 1H), 7.83 (s, 1H), 7.62-7.58 (m, 2H), 5.29 (s, 1H), 3.87 (s, 3H).

### Example 252: {3-[8-amino-5-(quinolin-6-yl)imidazo[1,2-a]pyrazin-6-yl]phenyl}methanol

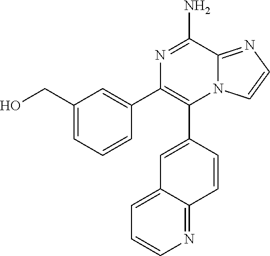 [SMILES: Nc1nc(-c2cccc(CO)c2)c(-c2ccc3ncccc3c2)n2ccnc12]

The title compound was synthesized following the approach outlined in Procedure F, substituting (quinolin-6-yl)boronic acid for (8-fluoroquinolin-6-yl)boronic acid in step (a), substituting [3-(hydroxymethyl)phenyl]boronic acid for 3-pyridineboronic acid pinacol ester in step (e) performing this step with 2 equiv. of boronic acid for 24 h to afford the title compound as a yellow powder (3.0 mg, 2.6%). ESI-MS: 368.2 [M+H]+. 1H NMR (400 MHz, DMSO-d6) δ 8.99 (dd, J=4.3, 1.7 Hz, 1H), 8.40 (d, J=8.1 Hz, 1H), 8.11 (d, J=1.9 Hz, 1H), 8.08 (d, J=8.7 Hz, 1H), 7.73 (dd, J=8.6, 2.0 Hz, 2H), 7.61 (dd, J=8.3, 4.3 Hz, 2H), 7.45 (s, 1H), 7.19 (d, J=7.6 Hz, 1H), 7.11 (dt, J=15.0, 7.8 Hz, 2H), 4.40 (s, 2H).

### Example 253: 6-(5-fluoropyridin-3-yl)-5-(quinolin-6-yl)imidazo[1,2-a]pyrazin-8-amine

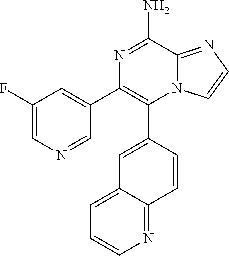 [SMILES: Nc1nc(-c2cncc(F)c2)c(-c2ccc3ncccc3c2)n2ccnc12]

The title compound was synthesized following the approach outlined in Procedure F, substituting (quinolin-6-yl)boronic acid for (8-fluoroquinolin-6-yl)boronic acid in step (a), substituting 5-fluoropyridine-3-boronic acid for 3-pyridineboronic acid pinacol ester in step (e) to afford the title compound as a beige solid (3.7 mg, 3.5%). ESI-MS: 357.2 [M+H]+. 1H NMR (400 MHz, DMSO-d6) δ 8.99 (dd, J=4.2, 1.7 Hz, 1H), 8.38-8.34 (m, 2H), 8.22-8.20 (m, 1H), 8.13-8.09 (m, 2H), 7.78 (dd, J=8.7, 2.0 Hz, 1H), 7.67-7.63 (m, 1H), 7.61-7.57 (m, 2H), 7.50 (d, J=1.2 Hz, 1H), 7.32 (s, 2H).

### Example 254: 6-(6-fluoropyridin-3-yl)-5-(quinolin-6-yl)imidazo[1,2-a]pyrazin-8-amine

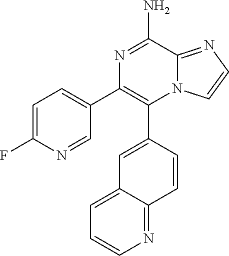 [SMILES: Nc1nc(-c2ccc(F)nc2)c(-c2ccc3ncccc3c2)n2ccnc12]

The title compound was synthesized following the approach outlined in Procedure F, substituting (quinolin-6-yl)boronic acid for (8-fluoroquinolin-6-yl)boronic acid in step (a), substituting (6-fluoropyridin-3-yl)boronic acid for 3-pyridineboronic acid pinacol ester in step (e) performing this step for 24 h at 100° C. to afford the title compound as a pale yellow solid (8 mg, 7.6%). ESI-MS: 357.2 [M+H]+. 1H NMR (400 MHz, DMSO-d6) δ 8.98 (dd, J=4.2, 1.7 Hz, 1H), 8.36 (dd, J=8.3, 1.7 Hz, 1H), 8.15-8.04 (m, 3H), 7.86 (td, J=8.3, 2.5 Hz, 1H), 7.76 (dd, J=8.7, 2.0 Hz, 1H), 7.59 (dd, J=8.3, 4.2 Hz, 1H), 7.57 (d, J=1.2 Hz, 1H), 7.49 (d, J=1.2 Hz, 1H), 7.29 (s, 2H), 7.04 (dd, J=8.6, 2.8 Hz, 1H).

### Example 255: 6-(4-fluorophenyl)-5-(quinolin-6-yl)imidazo[1,2-a]pyrazin-8-amine

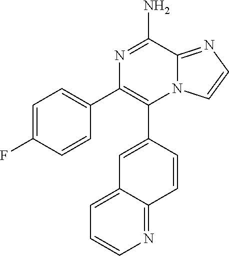 [SMILES: Nc1nc(-c2ccc(F)cc2)c(-c2ccc3ncccc3c2)n2ccnc12]

The title compound was synthesized following the approach outlined in Procedure F, substituting (quinolin-6-yl)boronic acid for (8-fluoroquinolin-6-yl)boronic acid in step (a), substituting 4-fluorophenylboronic acid for 3-pyridineboronic acid pinacol ester in step (e) to afford the title compound as a hydrochloride salt (yellow solid, 50 mg, 58.7%). ESI-MS: 356.2 [M+H]+. 1H NMR (300 MHz, DMSO-d6) δ 9.12 (dd, J=4.6, 1.7 Hz, 1H), 8.66 (d, J=8.0 Hz, 1H), 8.29-8.18 (m, 2H), 7.92 (d, J=1.3 Hz, 1H), 7.86-7.81 (m, 1H), 7.81-7.72 (m, 2H), 7.46-7.37 (m, 2H), 7.20-7.10 (m, 2H).

### Example 256: 5-(quinolin-6-yl)-6-[3-(trifluoromethyl)phenyl]imidazo[1,2-a]pyrazin-8-amine

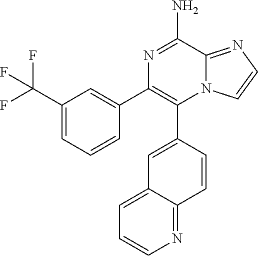 [SMILES: Nc1nc(-c2cccc(C(F)(F)F)c2)c(-c2ccc3ncccc3c2)n2ccnc12]

The title compound was synthesized following the approach outlined in Procedure F, substituting (quinolin-6-yl)boronic acid for (8-fluoroquinolin-6-yl)boronic acid in step (a), substituting 4,4,5,5-tetramethyl-2-[3-(trifluoromethyl)phenyl]-1,3,2-dioxaborolane for 3-pyridineboronic acid pinacol ester in step (e) to afford the title compound as a hydrochloride salt (yellow solid, 63 mg, 73.9%). ESI-MS: 406.2 [M+H]+. 1H NMR (300 MHz, DMSO-d6) δ 9.12 (dd, J=4.5, 1.7 Hz, 1H), 8.64 (d, J=8.0 Hz, 1H), 8.30-8.19 (m, 2H), 7.94 (d, J=1.4 Hz, 1H), 7.86 (dd, J=8.7, 2.0 Hz, 1H), 7.82-7.74 (m, 2H), 7.72 (d, J=2.0 Hz, 1H), 7.68-7.57 (m, 2H), 7.48 (t, J=7.8 Hz, 1H).

### Example 257: 6-(3-aminophenyl)-5-(8-fluoroquinolin-6-yl)imidazo[1,2-a]pyrazin-8-amine

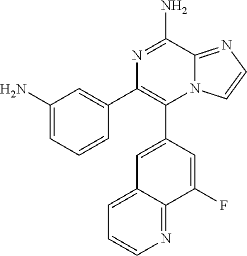 [SMILES: Nc1cccc(-c2nc(N)c3nccn3c2-c2cc(F)c3ncccc3c2)c1]

The title compound was synthesized following the approach outlined in Procedure F, substituting 3-aminophenylboronic acid for 3-pyridineboronic acid pinacol ester in step (e) performing this step using Sphos Pd G3 (0.06 equiv.) and K₂CO₃ (3.0 equiv.) for 24 h to afford the title compound as an orange solid (8 mg, 13.9%). ESI-MS: 371.2 [M+H]+. 1H NMR (300 MHz, DMSO-d6) δ 8.99 (dd, J=4.2, 1.6 Hz, 1H), 8.40 (dt, J=8.6, 1.6 Hz, 1H), 7.86 (d, J=1.7 Hz, 1H), 7.65 (dd, J=8.4, 4.2 Hz, 1H), 7.58 (dd, J=11.5, 1.7 Hz, 1H), 7.54-7.49 (m, 2H), 7.10 (s, 2H), 6.76-6.66 (m, 2H), 6.38-6.26 (m, 2H), 4.95 (s, 2H).

### Example 258: 3-[8-amino-5-(8-fluoroquinolin-6-yl)imidazo[1,2-a]pyrazin-6-yl]phenol

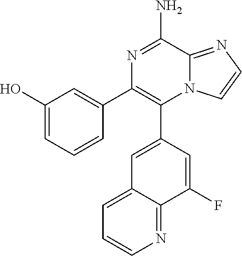 [SMILES: Nc1nc(-c2cccc(O)c2)c(-c2cc(F)c3ncccc3c2)n2ccnc12]

The title compound was synthesized following the approach outlined in Procedure F, substituting (3-hydroxyphenyl)boronic acid for 3-pyridineboronic acid pinacol ester in step (e) performing this step using Sphos Pd G3 (0.06 equiv.) and K₂CO₃ (3.0 equiv.) for 24 h to afford the title compound as a hydrochloride salt (yellow solid, 30 mg, 52.1%). ESI-MS: 372.2 [M+H]+. 1H NMR (300 MHz, DMSO-d6) δ 9.04 (dd, J=4.2, 1.6 Hz, 1H), 8.46 (dt, J=8.4, 1.6 Hz, 1H), 7.90 (dd, J=8.0, 1.5 Hz, 2H), 7.81 (d, J=1.3 Hz, 1H), 7.74-7.63 (m, 2H), 7.09 (t, J=7.9 Hz, 1H), 6.84-6.68 (m, 3H).

### Example 259: 6-(1,3-benzothiazol-6-yl)-5-(8-fluoroquinolin-6-yl)imidazo[1,2-a]pyrazin-8-amine

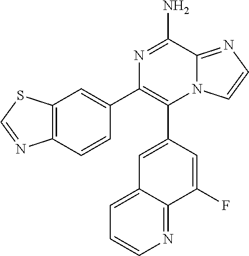 [SMILES: Nc1nc(-c2ccc3ncsc3c2)c(-c2cc(F)c3ncccc3c2)n2ccnc12]

The title compound was synthesized following the approach outlined in Procedure F, substituting benzothiazole-6-boronic acid pinacol ester for 3-pyridineboronic acid pinacol ester in step (e) to afford the title compound as a hydrochloride salt (yellow solid, 95 mg, 82%). ESI-MS: 413.2 [M+H]+. 1H NMR (400 MHz, DMSO-d6) δ 9.44 (s, 1H), 9.02 (dd, J=4.2, 1.6 Hz, 1H), 8.43 (dt, J=8.5, 1.6 Hz, 1H), 8.31 (d, J=1.7 Hz, 1H), 8.05-7.91 (m, 3H), 7.87 (d, J=1.4 Hz, 1H), 7.72 (dd, J=11.1, 1.7 Hz, 1H), 7.67 (dd, J=8.4, 4.2 Hz, 1H), 7.48 (dd, J=8.5, 1.8 Hz, 1H).

### Example 260: 5-(8-fluoroquinolin-6-yl)-6-(4-methoxyphenyl)imidazo[1,2-a]pyrazin-8-amine

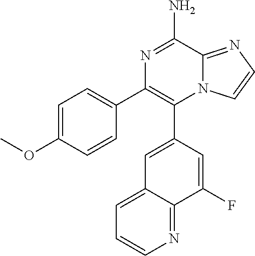 [SMILES: COc1ccc(-c2nc(N)c3nccn3c2-c2cc(F)c3ncccc3c2)cc1]

The title compound was synthesized following the approach outlined in Procedure F, substituting 4-methoxyphenylboronic acid for 3-pyridineboronic acid pinacol ester in step (e) to afford the title compound as a hydrochloride salt (yellow solid, 48 mg, 89%). ESI-MS: 386.2 [M+H]+. 1H NMR (400 MHz, DMSO-d6) δ 9.05 (dd, J=4.2, 1.6 Hz, 1H), 8.47 (d, J=8.4 Hz, 1H), 7.94 (d, J=1.7 Hz, 1H), 7.90 (s, 1H), 7.82 (d, J=1.2 Hz, 1H), 7.71 (dd, J=8.4, 4.2 Hz, 1H), 7.66 (dd, J=11.1, 1.7 Hz, 1H), 7.36-7.28 (m, 2H), 6.95-6.85 (m, 2H), 3.70 (s, 3H).

### Example 261: 5-(8-fluoroquinolin-6-yl)-6-(1H-pyrazol-5-yl)imidazo[1,2-a]pyrazin-8-amine

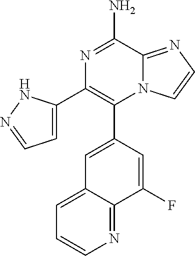 [SMILES: Nc1nc(-c2ccn[nH]2)c(-c2cc(F)c3ncccc3c2)n2ccnc12]

The title compound was synthesized following the approach outlined in Procedure F, substituting 1H-pyrazole-3-boronic acid pinacol ester for 3-pyridineboronic acid pinacol ester in step (e) to afford the title compound as a hydrochloride salt (yellow solid, 4 mg, 15.8%). ESI-MS: 346.2 [M+H]+. 1H NMR (400 MHz, DMSO-d6) δ 9.13 (dd, J=4.2, 1.6 Hz, 1H), 8.58 (d, J=8.4 Hz, 1H), 8.12-8.09 (m, 1H), 7.83-7.75 (m, 3H), 7.71-7.69 (m, 1H), 7.66 (d, J=2.5 Hz, 1H), 5.37-5.32 (m, 1H).

### Example 262: 3-[8-amino-5-(quinolin-6-yl)imidazo[1,2-a]pyrazin-6-yl]benzonitrile

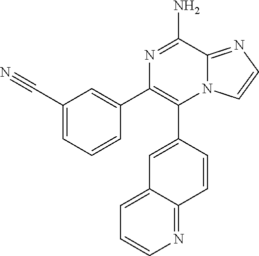 [SMILES: N#Cc1cccc(-c2nc(N)c3nccn3c2-c2ccc3ncccc3c2)c1]

The title compound was synthesized following the approach outlined in Procedure F, substituting (quinolin-6-yl)boronic acid for (8-fluoroquinolin-6-yl)boronic acid in step (a), substituting 3-cyanophenylboronic acid for 3-pyridineboronic acid pinacol ester in step (e) performing this step at 120° C. using 2.0 equiv. of 3-cyanophenylboronic acid and 0.1 equiv. of Pd(PPh₃)₄ to afford the title compound as a hydrochloride salt (yellow solid, 15 mg, 28.6%). ESI-MS: 363.2 [M+H]+. 1H NMR (400 MHz, DMSO-d6) δ 9.03 (dd, J=4.3, 1.7 Hz, 1H), 8.46 (dd, J=8.6, 1.8 Hz, 1H), 8.15 (d, J=2.0 Hz, 1H), 8.12 (d, J=8.7 Hz, 1H), 7.84 (t, J=1.7 Hz, 1H), 7.79 (dd, J=8.7, 2.0 Hz, 1H), 7.73-7.68 (m, 2H), 7.66 (dd, J=8.3, 4.3 Hz, 1H), 7.60 (d, J=1.2 Hz, 1H), 7.55 (dt, J=8.0, 1.5 Hz, 1H), 7.39 (t, J=7.8 Hz, 1H).

### Example 263: 5-[8-amino-6-(5-methylfuran-2-yl)imidazo[1,2-a]pyrazin-5-yl]-1-ethyl-1,2-dihydropyridin-2-one

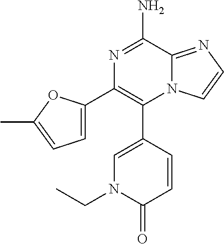 [SMILES: CCn1cc(-c2c(-c3ccc(C)o3)nc(N)c3nccn23)ccc1=O]

The title compound was synthesized following the approach outlined in Procedure F, substituting 1-ethyl-5-(tetramethyl-1,3,2-dioxaborolan-2-yl)-1,2-dihydropyridin-2-one for (8-fluoroquinolin-6-yl)boronic acid in step (a), substituting 5-methyl-2-furanboronic acid pinacol ester for 3-pyridineboronic acid pinacol ester in step (e) to afford the title compound as a hydrochloride salt (yellow solid, 11 mg, 43.8%). ESI-MS: 336.2 [M+H]+. 1H NMR (400 MHz, DMSO-d6) δ 7.99 (d, J=2.5 Hz, 1H), 7.75 (d, J=8.5 Hz, 2H), 7.43 (dd, J=9.3, 2.6 Hz, 1H), 6.57 (d, J=9.3 Hz, 1H), 6.41 (d, J=3.3 Hz, 1H), 6.22-6.16 (m, 1H), 4.13-3.80 (m, 2H), 2.21 (s, 3H), 1.24 (t, J=7.1 Hz, 3H).

### Example 264: 6-(5-chloro-6-methoxypyridin-3-yl)-5-(quinolin-6-yl)imidazo[1,2-a]pyrazin-8-amine

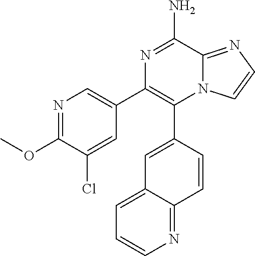 [SMILES: COc1ncc(-c2nc(N)c3nccn3c2-c2ccc3ncccc3c2)cc1Cl]

The title compound was synthesized following the approach outlined in Procedure F, substituting (quinolin-6-yl)boronic acid for (8-fluoroquinolin-6-yl)boronic acid in step (a), substituting 3-chloro-2-methoxy-5-(tetramethyl-1,3,2-dioxaborolan-2-yl)pyridine for 3-pyridineboronic acid pinacol ester in step (e) performing this step at 100° C. to afford the title compound as a yellow solid (120 mg, 45.6%). ESI-MS: 403.2 [M+H]+. 1H NMR (400 MHz, DMSO-d6) δ 8.99 (dd, J=4.2, 1.7 Hz, 1H), 8.39 (ddd, J=8.4, 1.7, 0.7 Hz, 1H), 8.16 (d, J=2.0 Hz, 1H), 8.12 (d, J=8.6 Hz, 1H), 7.91 (d, J=2.1 Hz, 1H), 7.82 (d, J=2.1 Hz, 1H), 7.78 (dd, J=8.7, 2.0 Hz, 1H), 7.60 (dd, J=8.3, 4.2 Hz, 1H), 7.55 (d, J=1.2 Hz, 1H), 7.45 (d, J=1.2 Hz, 1H), 7.27 (s, 2H), 3.81 (s, 3H).

### Example 265:1-{5-[8-amino-5-(quinolin-6-yl)imidazo[1,2-a]pyrazin-6-yl]pyridin-2-yl}ethan-1-one

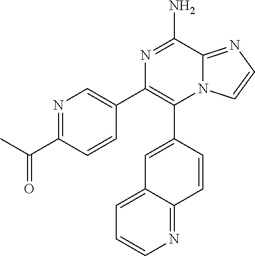 [SMILES: CC(=O)c1ccc(-c2nc(N)c3nccn3c2-c2ccc3ncccc3c2)cn1]

The title compound was synthesized following the approach outlined in Procedure F, substituting (quinolin-6-yl)boronic acid for (8-fluoroquinolin-6-yl)boronic acid in step (a), substituting 1-[5-(tetramethyl-1,3,2-dioxaborolan-2-yl)pyridin-2-yl]ethan-1-one for 3-pyridineboronic acid pinacol ester in step (e) performing this step at 100° C. to afford the title compound as a hydrochloride salt (orange solid, 13 mg, 11.6%). ESI-MS: 381.4 [M+H]+. 1H NMR (400 MHz, DMSO-d6) δ 9.13-9.09 (m, 1H), 8.63-8.56 (m, 2H), 8.30-8.25 (m, 1H), 8.21 (d, J=8.8 Hz, 1H), 7.95 (dd, J=8.2, 2.1 Hz, 1H), 7.90-7.81 (m, 3H), 7.75 (dd, J=8.4, 4.5 Hz, 1H), 7.68 (s, 1H), 2.53 (s, 3H).

### Example 266: 6-(3,4-difluorophenyl)-5-(4-methylquinolin-6-yl)imidazo[1,2-a]pyrazin-8-amine

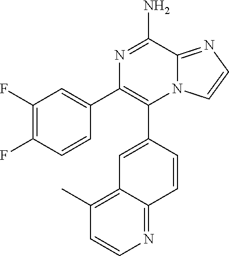 [SMILES: Cc1ccnc2ccc(-c3c(-c4ccc(F)c(F)c4)nc(N)c4nccn34)cc12]

The title compound was synthesized following the approach outlined in Procedure F, substituting (4-methylquinolin-6-yl)boronic acid (example 129) for (8-fluoroquinolin-6-yl)boronic acid in step (a), substituting 3,4-difluorophenylboronic acid for 3-pyridineboronic acid pinacol ester in step (e) to afford the title compound as a hydrochloride salt (yellow solid, 33 mg, 11.6%). ESI-MS: 388.1 [M+H]+. 1H NMR (400 MHz, DMSO-d6) δ 9.02 (d, J=4.9 Hz, 1H), 8.42 (d, J=1.9 Hz, 1H), 8.21 (d, J=8.7 Hz, 1H), 7.85 (dd, J=8.7, 1.9 Hz, 1H), 7.79 (d, J=1.3 Hz, 1H), 7.75-7.68 (m, 2H), 7.47 (td, J=9.8, 2.1 Hz, 1H), 7.30 (q, J=9.4 Hz, 1H), 7.12 (dt, J=8.6, 2.4 Hz, 1H), 2.73 (s, 3H).

### Example 267: 5-(4-methylquinolin-6-yl)-6-(1,3-thiazol-4-yl)imidazo[1,2-a]pyrazin-8-amine

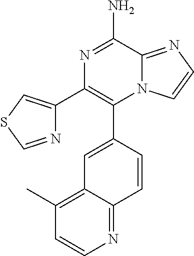 [SMILES: Cc1ccnc2ccc(-c3c(-c4cscn4)nc(N)c4nccn34)cc12]

The title compound was synthesized following the approach outlined in Procedure F, substituting (4-methylquinolin-6-yl)boronic acid (example 129) for (8-fluoroquinolin-6-yl)boronic acid in step (a), substituting 4-(tributylstannyl)-1,3-thiazole for 3-pyridineboronic acid pinacol ester in step (e) performing this step without any base and with 3.0 equiv of 4-(tributylstannyl)-1,3-thiazole at 100° C. for 24 h to afford the title compound as a hydrochloride salt (yellow solid, 45 mg, 57%). ESI-MS: 359.2 [M+H]+. 1H NMR (400 MHz, DMSO-d6) δ 9.20 (d, J=5.2 Hz, 1H), 9.12 (d, J=1.9 Hz, 1H), 8.62 (d, J=1.8 Hz, 1H), 8.51 (d, J=8.7 Hz, 1H), 8.04 (dd, J=8.8, 1.8 Hz, 1H), 7.93 (d, J=5.3 Hz, 1H), 7.89 (d, J=1.3 Hz, 1H), 7.67 (d, J=1.3 Hz, 1H), 7.36 (s, 1H), 2.85 (s, 3H).

### Procedure G: Example 268: 8-amino-6-(3-fluorophenyl)-5-(4-methylquinolin-6-yl)imidazo[1,2-a]pyrazine-2-carboxamide

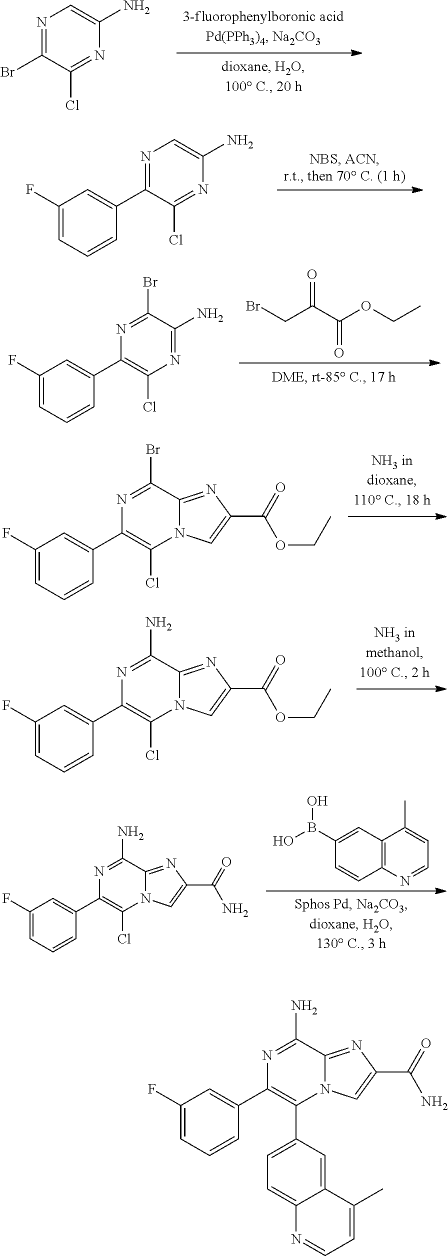

### a. 6-chloro-5-(3-fluorophenyl)pyrazin-2-amine

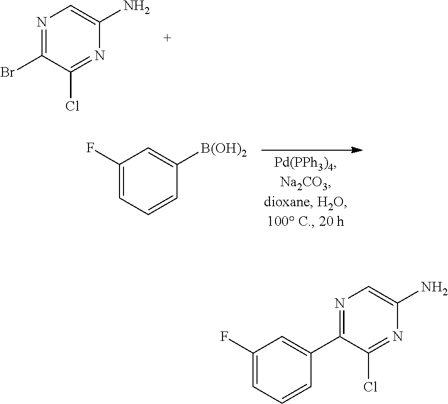

In a pressure tube were mixed 2-amino-5-bromo-6-chloropyrazine (2.00 g, 9.60 mmol), 3-fluorophenylboronic acid (1.48 g, 10.55 mmol), sodium carbonate (2.03 g, 19.19 mmol) in a 4:1 mixture of 1,4-dioxane:water (25 mL). The reaction mixture was sparged with argon and Pd(PPh₃)₄ (0.22 g, 0.20 mmol) was then added. The mixture was sparged with argon shortly and the vessel was sealed and the reaction mixture was heated at 100° C. for 20 h. After that time the reaction mixture was cooled down to r.t. filtered through Celite® pad diluted with AcOEt, organic layer was washed with NaHCO₃, brine and dried over Na₂SO₄. Then the mixture was concentrated under reduced pressure. The obtained residue was purified by flash chromatography on silica eluting with hexane:EtOAc (1:0-1:1) to lead to the title product as a light yellow solid (1.82 g, 85%). ESI-MS: 224.00 [M+H]+.

### b. 3-bromo-6-chloro-5-(3-fluorophenyl)pyrazin-2-amine

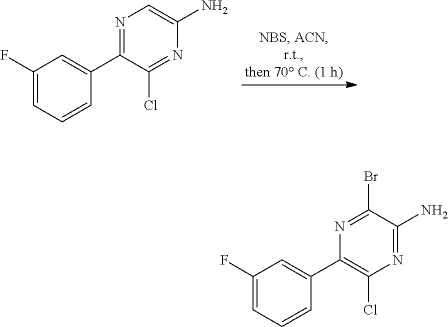 [SMILES: Nc1cnc(-c2cccc(F)c2)c(Cl)n1 ; Nc1nc(Cl)c(-c2cccc(F)c2)nc1Br]

N-Bromosuccinimide (0.587 g, 3.30 mmol) was added in 3 portions to the solution of 6-chloro-5-(3-fluorophenyl)pyrazin-2-amine (0.700 g, 3.00 mmol) in CH₃CN (10 mL). The reaction mixture was allowed to warm to r.t. then was heated at 70° C. for 1 h. Then reaction mixture was concentrated under reduced pressure. Residue was dissolved in AcOEt and then water was added. Aqueous layer was extracted with EtOAc, then organic layers were washed with brine and dried over Na₂SO₄, filtered and evaporated. Product was purified by flash chromatography on silica eluting with hexane/EtOAc (0-20%) to afford the title product (0.750 g, 83%) as a light yellow solid. ESI-MS: 303.70 [M+H]+.

### c. ethyl 8-bromo-5-chloro-6-(3-fluorophenyl)imidazo[1,2-a]pyrazine-2-carboxylate

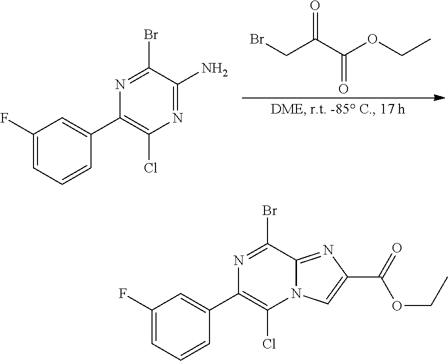

To the 3-bromo-6-chloro-5-(3-fluorophenyl)pyrazin-2-amine (0.5 g; 1.65 mmol) dissolved in DME (5 ml) was added 3-bromopyruvic acid ethyl ester (1.29 g; 6.61 mmol). Reaction was stirred for 1 h in r.t. then heated at 85° C. for 16 h. After cooling to r.t. reaction mixture was diluted with DCM and washed with sat. NaHCO₃ (aq). Combined organic layers were dried over MgSO₄. Product was purified by column chromatography eluting with DCM/EtOH (0-5%) to afford the title product as a white solid (0.190 g, 26%). ESI-MS: 400.05 [M+H]+.

### d. ethyl 8-amino-5-chloro-6-(3-fluorophenyl)imidazo[1,2-a]pyrazine-2-carboxylate

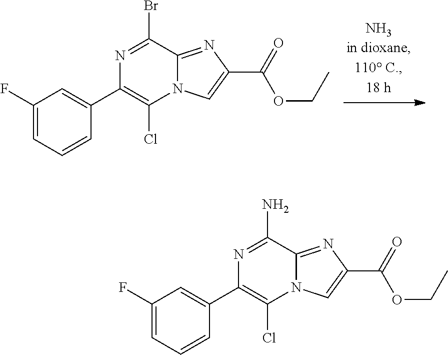 [SMILES: CCOC(=O)c1cn2c(Cl)c(-c3cccc(F)c3)nc(Br)c2n1 ; CCOC(=O)c1cn2c(Cl)c(-c3cccc(F)c3)nc(N)c2n1]

To the ethyl 8-bromo-5-chloro-6-(3-fluorophenyl)imidazo[1,2-a]pyrazine-2-carboxylate (1 g, 2.51 mmol) 0.5N ammonium in dioxane (100 ml) was added. Reaction was heated for 20 h at 110° C. After cooling to r.t. reaction mixture was concentrated under reduced pressure and remaining residue was purified by flash chromatography eluting with DCM/EtOH (0-5%) to afford the title product as a white solid (0.84 g, 2.51 mmol, quant.). ESI-MS: 335.15 [M+H]+.

### e. 8-amino-5-chloro-6-(3-fluorophenyl)imidazo[1,2-a]pyrazine-2-carboxamide

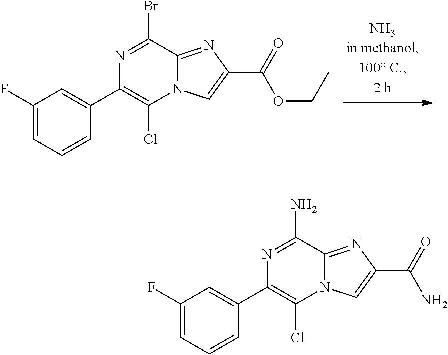 [SMILES: CCOC(=O)c1cn2c(Cl)c(-c3cccc(F)c3)nc(Br)c2n1 ; NC(=O)c1cn2c(Cl)c(-c3cccc(F)c3)nc(N)c2n1]

To the ethyl 8-amino-5-chloro-6-(3-fluorophenyl)imidazo[1,2-a]pyrazine-2-carboxylate (0.84 g, 2.51 mmol) 7N ammonium in methanol (100 ml) was added and reaction was heated for 2 h in 100° C. After cooling to r.t. reaction mixture was concentrated and remaining residue was purified by flash chromatography eluting with DCM/MeOH (0-10%) to afford the title product as a pale yellow solid (0.78 g, 2.51 mmol, quant.). ESI-MS: 305.85 [M+H]+.

### f. 8-amino-6-(3-fluorophenyl)-5-(4-methylquinolin-6-yl)imidazo[1,2-a]pyrazine-2-carboxamide

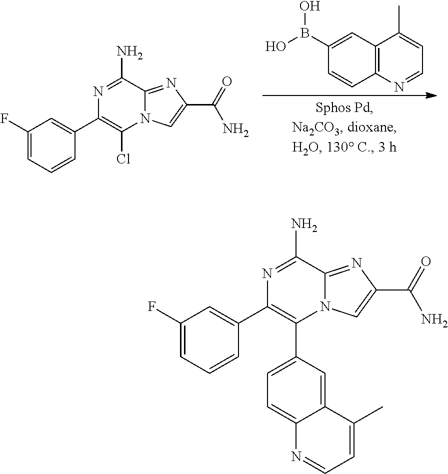 [SMILES: NC(=O)c1cn2c(Cl)c(-c3cccc(F)c3)nc(N)c2n1 ; Cc1ccnc2ccc(B(O)O)cc12 ; Cc1ccnc2ccc(-c3c(-c4cccc(F)c4)nc(N)c4nc(C(N)=O)cn34)cc12]

To the 8-amino-5-chloro-6-(3-fluorophenyl)imidazo[1,2-a]pyrazine-2-carboxamide (8 mg; 0.03 mmol) dissolved in dioxane/water 4:1 (10 ml) was added (4-methylquinolin-6-yl)boronic acid (example 129) (6 mg, 0.03 mmol) and sodium carbonate (8 mg, 0.08 mmol) followed by Sphos Pd G3 (2 mg, 0.1 mmol) and the mixture was sparged with argon for 15 min. Reaction mixture was heated at 130° C. for 3 h. After that time the reaction mixture was cooled to r.t. then filtered through a pad of Celite® and concentrated under reduced pressure. Residue was purified by flash chromatography on silica eluting with DCM/MeOH (0-10%) to afford the title product (10 mg, 93%) as a white solid. ESI-MS: 413.3 [M+H]+. 1H NMR (400 MHz, DMSO-d6) δ 8.82 (d, J=4.4 Hz, 1H), 8.20 (d, J=1.7 Hz, 1H), 8.08 (d, J=8.6 Hz, 1H), 7.83 (s, 1H), 7.76 (dd, J=8.6, 1.9 Hz, 1H), 7.55 (s, 1H), 7.46 (s, 1H), 7.43 (d, J=4.4 Hz, 1H), 7.33 (s, 2H), 7.17 (dt, J=6.2, 5.1 Hz, 2H), 7.08 (d, J=8.0 Hz, 1H), 7.05-6.94 (m, 1H), 2.55 (s, 3H).

### Example 269: 8-amino-6-(3-fluorophenyl)-N-methyl-5-{3-methylimidazo[1,2-a]pyridin-6-yl}imidazo[1,2-a]pyrazine-2-carboxamide

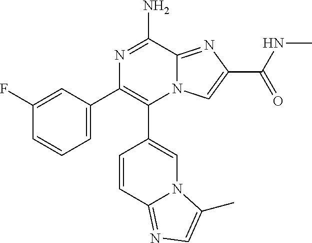 [SMILES: CNC(=O)c1cn2c(-c3ccc4ncc(C)n4c3)c(-c3cccc(F)c3)nc(N)c2n1]

The title compound was synthesized following the approach outlined in Procedure G substituting methylamine (33% in EtOH) for 7N NH₃ in methanol in step (e) and substituting (1-methyl-1H-benzimidazol-6-yl)boronic acid for (4-methylquinolin-6-yl)boronic acid in step (f) to lead to the title compound (3 mg, 10%) as a beige solid as a hydrochloride salt. ESI-MS: 416.30 [M+H]+. ¹H NMR (400 MHz, DMSO-d6) δ 9.14 (s, 1H), 8.16 (s, 1H), 8.11 (dd, J=4.8 Hz, 1H), 8.07 (d, J=1.2 Hz, 1H), 7.99 (dd, J=9.3, 0.9 Hz, 1H), 7.79 (dd, J=9.2, 1.5 Hz, 1H), 7.52 (s, 2H), 7.33-7.06 (m, 4H), 2.81 (d, J=4.8 Hz, 3H), 2.53 (d, J=1.1 Hz, 3H).

### Example 270: 8-amino-6-(4-fluorophenyl)-5-(4-methylquinolin-6-yl)imidazo[1,2-a]pyrazine-2-carboxamide

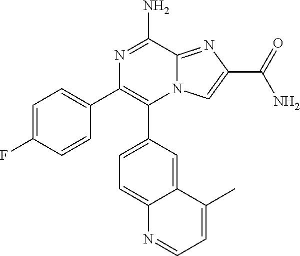 [SMILES: Cc1ccnc2ccc(-c3c(-c4ccc(F)cc4)nc(N)c4nc(C(N)=O)cn34)cc12]

The title compound was synthesized following the approach outlined in Procedure G substituting 4-fluorophenylboronic acid for 3-fluorophenylboronic acid in step (a) to afford the title compound as a hydrochloride salt (yellow solid, 12 mg, 15%). ESI-MS: 413.30 [M+H]+. 1H NMR (400 MHz, DMSO-d6) δ 9.08 (d, J=5.1 Hz, 1H), 8.47 (d, J=1.9 Hz, 1H), 8.26 (d, J=8.8 Hz, 1H), 8.00 (s, 1H), 7.91 (dd, J=8.8, 1.8 Hz, 1H), 7.81 (d, J=5.0 Hz, 1H), 7.57 (d, J=13.2 Hz, 2H), 7.43-7.35 (m, 2H), 7.14-7.04 (m, 2H), 2.76 (s, 3H).

### Example 271: 8-amino-6-(3-fluorophenyl)-5-{3-methylimidazo[1,2-a]pyridin-6-yl}imidazo[1,2-a]pyrazine-2-carboxamide

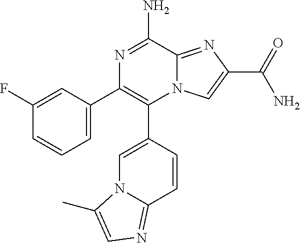 [SMILES: Cc1cnc2ccc(-c3c(-c4cccc(F)c4)nc(N)c4nc(C(N)=O)cn34)cn12]

The title compound was synthesized following the approach outlined in Procedure G substituting (1-methyl-1H-benzimidazol-6-yl)boronic acid for (4-methylquinolin-6-yl)boronic acid in step (f) to lead to the title compound (5 mg, 10%) as a grey solid. ESI-MS: 402.30 [M+H]+. 1H NMR (400 MHz, DMSO-d6) δ 9.11 (s, 1H), 8.16 (s, 1H), 8.04 (s, 1H), 7.98 (d, J=9.2 Hz, 1H), 7.76 (d, J=9.4 Hz, 1H), 7.53 (s, 2H), 7.48 (s, 2H), 7.36-7.19 (m, 2H), 7.20-7.11 (m, 1H), 7.12-7.00 (m, 1H), 2.53 (s, 3H).

### Example 272:8-amino-6-(4-fluorophenyl)-N-methyl-5-(4-methylquinolin-6-yl)imidazo[1,2-a]pyrazine-2-carboxamide

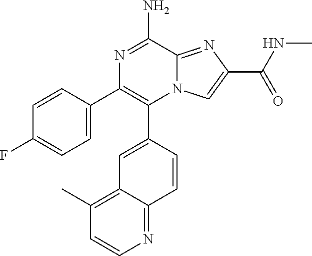 [SMILES: CNC(=O)c1cn2c(-c3ccc4nccc(C)c4c3)c(-c3ccc(F)cc3)nc(N)c2n1]

The title compound was synthesized following the approach outlined in Procedure G substituting 4-fluorophenylboronic acid for 3-fluorophenylboronic acid in step (a) and substituting methylamine (33% in EtOH) for 7N NH₃ in methanol in step (e) and performing this step at 100° C. for 2 h and performing step (f) under microwave irradiation at 130° C. for 1 h to lead to the title compound (3.3 mg, 66%) as a yellow solid, hydrochloride salt. ESI-MS: 427.30 [M+H]+. 1H NMR (400 MHz, DMSO-d6) δ 9.06 (d, J=5.0 Hz, 1H), 8.44 (s, 1H), 8.23 (d, J=8.7 Hz, 1H), 8.18 (d, J=5.0 Hz, 1H), 7.93 (s, 1H), 7.90 (d, J=8.6 Hz, 1H), 7.77 (s, 1H), 7.42-7.34 (m, 2H), 7.08 (t, J=8.7 Hz, 2H), 2.78 (d, J=4.7 Hz, 3H), 2.74 (s, 3H).

### Example 273: 8-amino-6-(3-fluorophenyl)-5-(1-methyl-1H-1,3-benzodiazol-6-yl)imidazo[1,2-a]pyrazine-2-carboxamide

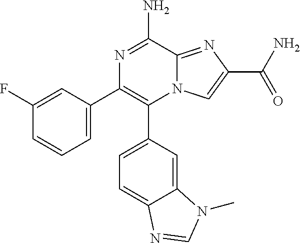 [SMILES: Cn1cnc2ccc(-c3c(-c4cccc(F)c4)nc(N)c4nc(C(N)=O)cn34)cc21]

The title compound was synthesized following the approach outlined in Procedure G substituting (1-methyl-1H-benzimidazol-6-yl)boronic acid for (4-methylquinolin-6-yl)boronic acid in step (f) to lead to the title compound (20 mg, 37%) as a yellow solid, hydrochloride salt. ESI-MS: 402.30 [M+H]+. 1H NMR (400 MHz, DMSO-d6) δ 8.94 (s, 2H), 8.01 (s, 1H), 7.83 (d, J=8.5 Hz, 1H), 7.69 (s, 1H), 7.52 (d, J=8.3 Hz, 2H), 7.37 (d, J=8.5 Hz, 1H), 7.28 (s, 1H), 7.25-7.13 (m, 2H), 7.13-6.97 (m, 2H), 3.93 (s, 3H).

### Example 274:8-amino-6-(4-fluorophenyl)-5-(quinolin-6-yl)imidazo[1,2-a]pyrazine-2-carboxamide

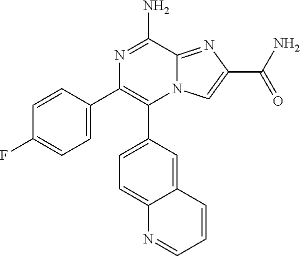 [SMILES: NC(=O)c1cn2c(-c3ccc4ncccc4c3)c(-c3ccc(F)cc3)nc(N)c2n1]

The title compound was synthesized following the approach outlined in Procedure G substituting 4-fluorophenylboronic acid for 3-fluorophenylboronic acid in step (a) and substituting (quinolin-6-yl)boronic acid for (4-methylquinolin-6-yl)boronic acid in step (f) and substituting Pd(amphos)Cl₂ for Sphos Pd G3 in step (f) and heating under microwave irradiation at 130° C. for 0.5 h to lead to the title compound (89 mg, 71%) as a yellow solid, hydrochloride salt. ESI-MS: 399.30 [M+H]+. 1H NMR (400 MHz, DMSO-d6) δ 9.15 (dd, J=4.7, 1.6 Hz, 1H), 8.90 (s, 2H), 8.74 (d, J=8.4 Hz, 1H), 8.30 (d, J=1.9 Hz, 1H), 8.26 (d, J=8.8 Hz, 1H), 8.03 (s, 1H), 7.88-7.79 (m, 2H), 7.63 (s, 1H), 7.59 (s, 1H), 7.44-7.37 (m, 2H), 7.17-7.10 (m, 2H).

### Example 275: ethyl 8-amino-6-(4-fluorophenyl)-5-(4-methylquinolin-6-yl)imidazo[1,2-a]pyrazine-2-carboxylate

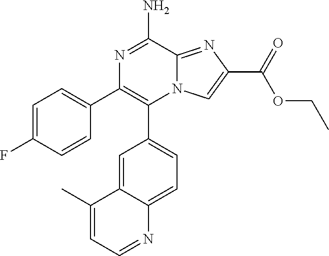 [SMILES: CCOC(=O)c1cn2c(-c3ccc4nccc(C)c4c3)c(-c3ccc(F)cc3)nc(N)c2n1]

The title compound was synthesized following the approach outlined in Procedure G substituting 4-fluorophenylboronic acid for 3-fluorophenylboronic acid in step (a) and excluding the step (e) and performing step (f) in dioxane/water 16:1 for 15 h at 125° C. to lead to the title compound (4 mg, 13%) as a yellow solid, hydrochloride salt. ESI-MS: 442.30 [M+H]+. 1H NMR (400 MHz, DMSO-d6) δ 9.04 (d, J=5.0 Hz, 1H), 8.39 (s, 1H), 8.24-8.20 (m, 1H), 7.96-7.90 (m, 2H), 7.74 (d, J=4.7 Hz, 1H), 7.39-7.32 (m, 2H), 7.09-7.01 (m, 2H), 4.29 (q, J=7.1 Hz, 2H), 2.71 (s, 3H), 1.26 (t, J=7.1 Hz, 3H).

### Example 276: ethyl 8-amino-6-(3-cyanophenyl)-5-(8-fluoroquinolin-6-yl)imidazo[1,2-a]pyrazine-2-carboxylate

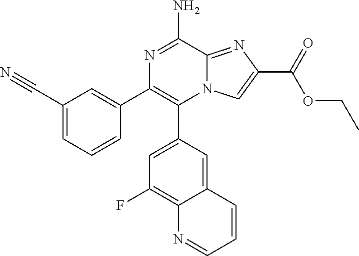 [SMILES: CCOC(=O)c1cn2c(-c3cc(F)c4ncccc4c3)c(-c3cccc(C#N)c3)nc(N)c2n1]

The title compound was synthesized following the approach outlined in Procedure G substituting 3-cyanophenylboronic acid for 3-fluorophenylboronic acid in step (a) and performing step (b) with THE at room temperature for 16 h and performing step (c) at 100° C. for 16 h and excluding the step (e) and in step (f) substituting 8-fluoro-6-(tetramethyl-1,3,2-dioxaborolan-2-yl)quinoline for (4-methylquinolin-6-yl)boronic acid and substituting Pd(amphos)Cl₂ for Sphos Pd G3 and performing this step in dioxane/water 10:1 for 1 h at 100° C. to lead to the title compound (4 mg, 6%) as a white solid. ESI-MS: 453.20 [M+H]+. 1H NMR (300 MHz, DMSO-d6) δ 9.03 (dd, J=4.3, 1.6 Hz, 1H), 8.42 (d, J=8.4 Hz, 1H), 7.98-7.91 (m, 2H), 7.84-7.81 (m, 1H), 7.76 (dd, J=11.3, 1.7 Hz, 1H), 7.72-7.58 (m, 4H), 7.56-7.48 (m, 1H), 7.41-7.32 (m, 1H), 4.29 (q, J=7.1 Hz, 2H), 1.26 (t, J=7.1 Hz, 3H).

### Example 277: 6-(4-fluorophenyl)-5-(4-methylquinolin-6-yl)-2-(trifluoromethyl)imidazo[1,2-a]pyrazin-8-amine

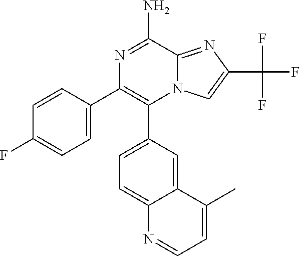 [SMILES: Cc1ccnc2ccc(-c3c(-c4ccc(F)cc4)nc(N)c4nc(C(F)(F)F)cn34)cc12]

The title compound was synthesized following the approach outlined in Procedure G substituting 4-fluorophenylboronic acid for 3-fluorophenylboronic acid in step (a) and substituting 1-bromo-3,3,3-trifluoroacetone for 3-bromopyruvic acid ethyl ester and heating in dioxane for 16 h at 110° C. in step (c) to lead to the title compound (13 mg, 28%) as a white solid. ESI-MS: 438.20 [M+H]+. 1H NMR (300 MHz, DMSO-d6) δ 8.80 (d, J=4.3 Hz, 1H), 8.19 (d, J=1.8 Hz, 1H), 8.06-8.00 (m, 2H), 7.70 (dd, J=8.7, 1.9 Hz, 1H), 7.54 (s, 2H), 7.41 (dd, J=4.4, 1.0 Hz, 1H), 7.36-7.29 (m, 2H), 7.05-6.96 (m, 2H), 2.56 (d, J=0.9 Hz, 3H).

### Example 278: 6-(4-fluorophenyl)-5-(quinolin-6-yl)-2-(trifluoromethyl)imidazo[1,2-a]pyrazin-8-amine

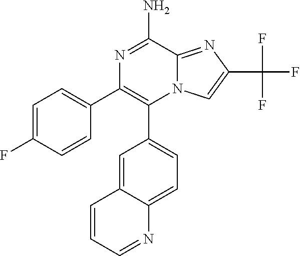 [SMILES: Nc1nc(-c2ccc(F)cc2)c(-c2ccc3ncccc3c2)n2cc(C(F)(F)F)nc12]

The title compound was synthesized following the approach outlined in Procedure G substituting 4-fluorophenylboronic acid for 3-fluorophenylboronic acid in step (a) and substituting 1-bromo-3,3,3-trifluoroacetone for 3-bromopyruvic acid ethyl ester in step (c) and performing this reaction in dioxane for 16 h at 110° C. and substituting (quinolin-6-yl)boronic acid for (4-methylquinolin-6-yl)boronic acid in step (f) and performing this step for 15 h at 130° C. to lead to the title compound (12 mg, 28%) as a beige solid. ESI-MS: 424.20 [M+H]+. 1H NMR (300 MHz, DMSO-d6) δ 8.96 (dd, J=4.2, 1.7 Hz, 1H), 8.35 (dd, J=8.4, 1.5 Hz, 1H), 8.11 (d, J=1.9 Hz, 1H), 8.06 (d, J=8.7 Hz, 1H), 7.99 (d, J=1.1 Hz, 1H), 7.71 (dd, J=8.7, 2.0 Hz, 1H), 7.60-7.53 (m, 3H), 7.37-7.29 (m, 2H), 7.05-6.96 (m, 2H).

### Example 279: 8-amino-6-(4-fluorophenyl)-N-methyl-5-(1-methyl-1H-1,3-benzodiazol-6-yl)imidazo[1,2-a]pyrazine-2-carboxamide

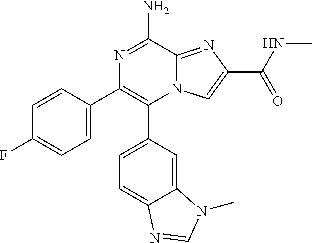 [SMILES: CNC(=O)c1cn2c(-c3ccc4ncn(C)c4c3)c(-c3ccc(F)cc3)nc(N)c2n1]

The title compound was synthesized following the approach outlined in Procedure G substituting 4-fluorophenylboronic acid for 3-fluorophenylboronic acid in step (a) and substituting methylamine (33% in EtOH) for 7N NH₃ in methanol and performing this step at 100° C. for 2 h in step (e) and substituting (1-methyl-1H-benzimidazol-6-yl)boronic acid for (4-methylquinolin-6-yl)boronic acid in step (f) and performing this step in a mixture of dioxane/water 10:1 at 130° C. for 1 h to lead to the title compound (10 mg, 9%) as an off-white solid, hydrochloride salt. ESI-MS: 416.30 [M+H]+. 1H NMR (400 MHz, DMSO-d6) δ 9.34 (s, 1H), 8.20-8.11 (m, 2H), 7.88 (d, J=8.8 Hz, 1H), 7.74 (s, 1H), 7.44 (dd, J=8.4, 1.5 Hz, 1H), 7.41-7.35 (m, 2H), 7.11-7.04 (m, 2H), 4.00 (s, 3H), 2.79 (d, J=4.7 Hz, 3H).

### Example 280: 6-(3-fluorophenyl)-5-(1-methyl-1H-1,3-benzodiazol-6-yl)-2-(morpholine-4-carbonyl)imidazo[1,2-a]pyrazin-8-amine

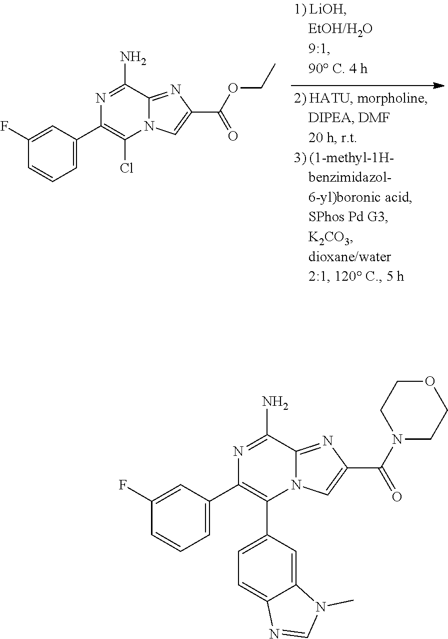 [SMILES: CCOC(=O)c1cn2c(Cl)c(-c3cccc(F)c3)nc(N)c2n1 ; Cn1cnc2ccc(-c3c(-c4cccc(F)c4)nc(N)c4nc(C(=O)N5CCOCC5)cn34)cc21]

Step 1) In a pressure tube was placed ethyl 8-amino-5-chloro-6-(3-fluorophenyl)imidazo[1,2-a]pyrazine-2-carboxylate [prepared following the approach outlined in Procedure G, steps (a-d)](410 mg, 1.22 mmol) and LiOH monohydrate (154 mg, 3.7 mmol, 3 equiv.) followed by etanol/water 3:2 (40 mL). Reaction was heated for 4 h at 90° C. After cooling to r.t. reaction mixture was concentrated and residue was lyophilized with water to afford yellow solid (429 mg, lithium salt, quant.) which was used in next step without purification. ESI-MS: 306.9 [M+H]+.

Step 2) 8-Amino-5-chloro-6-(3-fluorophenyl)imidazo[1,2-a]pyrazine-2-carboxylic acid lithium salt (214 mg, 0.68 mmol) was dissolved in DMF (14 mL) and then HATU (312 mg, 0.82 mmol, 1.2 equiv.) was added and mixture was stirred for 10 minutes. Then DIPEA (0.36 mL, 2.05 mmol, 3.0 equiv.) was added followed by morpholine (0.07 mL, 0.75 mmol, 1.1 equiv.) and reaction was stirred at r.t. for 20 h. Then reaction mixture was concentrated and water was added, aqueous phase was extracted with ethyl acetate. Organic layers were washed with brine then dried with Na₂SO₄, filtered and concentrated. Residue was purified by using flash chromatography (hexane/ethyl acetate) to give 5-chloro-6-(3-fluorophenyl)-2-(morpholine-4-carbonyl)imidazo[1,2-a]pyrazin-8-amine (96 mg, 37%) as a yellow solid. ESI-MS: 375.9 [M+H]+.

Step 3) The title compound was synthesized following the conditions from Procedure G, at step (f) substituting (1-methyl-1H-benzimidazol-6-yl)boronic acid for (4-methylquinolin-6-yl)boronic acid and performing this step at 120° C. for 5 h to lead to the title compound (19 mg, 16%) as a yellow solid, hydrochloride salt. ESI-MS: 472.30 [M+H]+. 1H NMR (400 MHz, DMSO-d6) δ 9.43 (s, 1H), 8.18 (s, 1H), 7.90 (d, J=8.1 Hz, 1H), 7.65 (s, 1H), 7.48 (d, J=8.5 Hz, 1H), 7.28-7.15 (m, 2H), 7.14-7.03 (m, 2H), 4.37-4.14 (m, 4H), 4.13-4.00 (m, 4H), 4.01 (s, 3H).

### Example 281: 6-(3-fluorophenyl)-5-(8-fluoroquinolin-6-yl)-2-[4-(4-methoxybenzoyl)piperazine-1-carbonyl]imidazo[1,2-a]pyrazin-8-amine

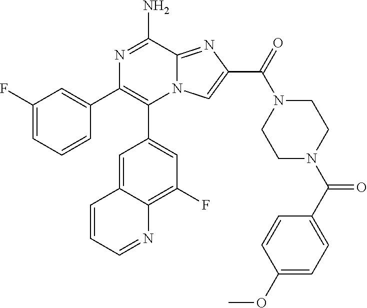 [SMILES: COc1ccc(C(=O)N2CCN(C(=O)c3cn4c(-c5cc(F)c6ncccc6c5)c(-c5cccc(F)c5)nc(N)c4n3)CC2)cc1]

The title compound was synthesized following approach from example 280, substituting 1-(4-methoxybenzoyl)piperazine for morpholine at step (2) and substituting 8-fluoro-6-(tetramethyl-1,3,2-dioxaborolan-2-yl)quinoline for (4-methylquinolin-6-yl)boronic acid and substituting Pd(dppf)Cl₂*DCM for Sphos Pd G3 in step (3). Product was purified by flash chromatography (DCM/MeOH 5%) to lead to the title compound (9 mg, 23%) as an off-white solid. ESI-MS: 620.30 [M+H]+. 1H NMR (400 MHz, Acetonitrile-d3) δ 8.98 (dd, J=4.1, 1.3 Hz, 1H), 8.25 (d, J=8.4 Hz, 1H), 7.78 (s, 2H), 7.60-7.55 (m, 1H), 7.46-7.38 (m, 3H), 7.17-7.10 (m, 2H), 7.07 (d, J=7.8 Hz, 1H), 7.01-6.90 (m, 3H), 6.15 (s, 2H), 4.29 (s, 2H), 3.82 (s, 3H), 3.77-3.51 (m, 6H).

### Example 282: 2-[4-(2,4-difluorophenyl)piperazine-1-carbonyl]-6-(3-fluorophenyl)-5-(1-methyl-1H-1,3-benzodiazol-6-yl)imidazo[1,2-a]pyrazin-8-amine

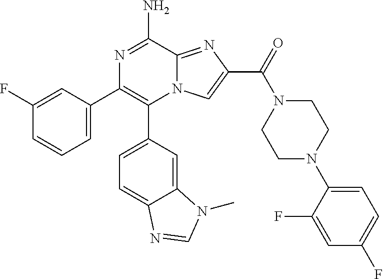 [SMILES: Cn1cnc2ccc(-c3c(-c4cccc(F)c4)nc(N)c4nc(C(=O)N5CCN(c6ccc(F)cc6F)CC5)cn34)cc21]

The title compound was synthesized following approach from example 280, substituting 1-(2,4-difluorophenyl)-piperazine for morpholine at step (2) and substituting Pd(dppf)Cl₂*DCM for Sphos Pd G3 in step (3) to lead to the title compound (20 mg, 22%) as an off-white solid, hydrochloride salt. ESI-MS: 583.30 [M+H]+. 1H NMR (300 MHz, DMSO-d6) δ 9.41 (s, 1H), 8.18 (d, J=1.5 Hz, 1H), 7.90 (d, J=8.4 Hz, 1H), 7.83 (s, 2H), 7.66 (s, 1H), 7.49 (dd, J=8.5, 1.5 Hz, 1H), 7.29-7.17 (m, 3H), 7.14-6.99 (m, 4H), 4.40-4.31 (m, 2H), 4.01 (s, 3H), 3.81-3.78 (m, 2H), 3.06-2.99 (m, 4H).

### Example 283: ethyl 8-amino-6-(4-fluorophenyl)-5-(quinolin-6-yl)imidazo[1,2-a]pyrazine-2-carboxylate

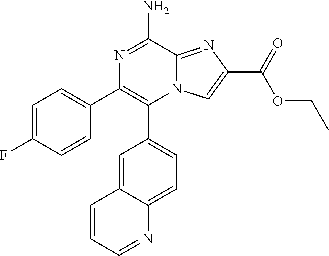 [SMILES: CCOC(=O)c1cn2c(-c3ccc4ncccc4c3)c(-c3ccc(F)cc3)nc(N)c2n1]

The title compound was synthesized following the approach outlined in Procedure G substituting 4-fluorophenylboronic acid for 3-fluorophenylboronic acid in step (a) and excluding step (e) and substituting (quinolin-6-yl)boronic acid for (4-methylquinolin-6-yl)boronic acid in step (f) and performing this step in a mixture of dioxane/water 16:1 heating under microwave irradiation at 130° C. for 2 h to lead to the title compound (8 mg, 42%) as a yellow solid, hydrochloride salt. ESI-MS: 428.30 [M+H]+. 1H NMR (400 MHz, DMSO-d6) δ 9.08 (dd, J=4.6, 1.6 Hz, 1H), 8.58 (d, J=8.4 Hz, 1H), 8.21-8.15 (m, 2H), 7.91 (s, 1H), 7.81 (dd, J=8.7, 1.9 Hz, 1H), 7.73 (dd, J=8.3, 4.6 Hz, 1H), 7.40-7.34 (m, 2H), 7.08 (t, J=8.9 Hz, 2H), 4.28 (q, J=7.1 Hz, 2H), 1.25 (t, J=7.1 Hz, 3H).

### Example 284: 1-[8-amino-6-(4-fluorophenyl)-5-(1-methyl-1H-1,3-benzodiazol-6-yl)imidazo[1,2-a]pyrazine-2-carbonyl]-4-methylpiperidin-4-ol

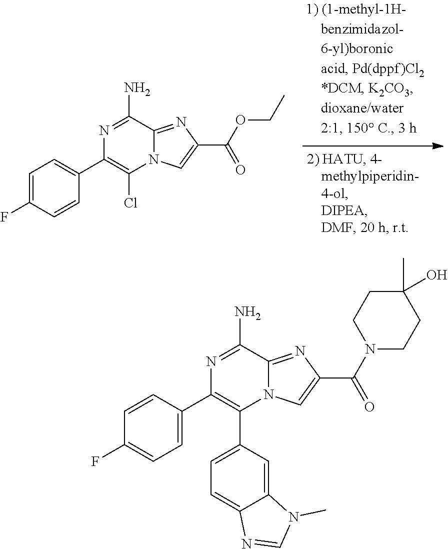 [SMILES: CCOC(=O)c1cn2c(Cl)c(-c3ccc(F)cc3)nc(N)c2n1 ; Cn1cnc2ccc(-c3c(-c4ccc(F)cc4)nc(N)c4nc(C(=O)N5CCC(C)(O)CC5)cn34)cc21]

Step 1) In a pressurized tube was placed ethyl 8-amino-5-chloro-6-(4-fluorophenyl)imidazo[1,2-a]pyrazine-2-carboxylate [prepared following the approach outlined in Procedure G, steps (a-d) except substituting 4-fluorophenylboronic acid for 3-fluorophenylboronic acid in step (a)] (100 mg, 0.3 mmol) followed by (1-methyl-1H-benzimidazol-6-yl)boronic acid (78 mg, 0.45 mmol, 1.5 equiv.) and K₂CO₃ (62 mg, 0.45 mmol, 1.5 equiv.) and Pd(dppf)Cl₂*CH₂Cl₂ (12 mg, 0.01 mmol, 0.05 equiv.). To this dioxane/water 4:1 mixture (5 mL) was added and suspension was sparged with argon for 15 min. After this reaction mixture was heated at 150° C. for 3 h. After cooling to r.t. reaction mixture was filtered through a Celite® pad filtrate was concentrated and residue was lyophilized to afford brown solid containing 8-amino-6-(4-fluorophenyl)-5-(1-methyl-1H-1,3-benzodiazol-6-yl)imidazo[1,2-a]pyrazine-2-carboxylic acid potassium salt (240 mg, quant.) which was used in next step without purification. ESI-MS: 402.95 [M+H]+.

Step 2) Residue containing 8-amino-6-(4-fluorophenyl)-5-(1-methyl-1H-1,3-benzodiazol-6-yl)imidazo[1,2-a]pyrazine-2-carboxylic acid potassium salt (120 mg, 0.28 mmol) was dissolved in DMF (3 mL) and then HATU (129 mg, 0.34 mmol, 1.2 equiv.) was added and mixture was stirred for 10 minutes. Then DIPEA (0.15 mL, 0.85 mmol, 3.0 equiv.) was added followed by 4-methylpiperidin-4-ol (0.04 mL, 0.31 mmol, 1.1 equiv.) and reaction was stirred at r.t. for 1 h. Then reaction mixture was concentrated and residue was purified by using flash chromatography followed by HPLC to give title product as a yellow solid (20 mg, 15%) as a hydrochloride salt. ESI-MS: 500.3 [M+H]+. 1H NMR (400 MHz, DMSO-d6) δ 9.34 (s, 1H), 8.13 (s, 1H), 8.0 (s, 2H), 7.86 (d, J=8.5 Hz, 1H), 7.63 (s, 1H), 7.47-7.33 (m, 3H), 7.08 (t, J=8.8 Hz, 2H), 4.45 (d, J=13.2 Hz, 1H), 4.06 (d, J=13.2 Hz, 2H), 3.99 (s, 3H), 3.21 (s, 2H), 1.47 (d, J=32.5 Hz, 4H), 1.16 (s, 3H).

### Example 285: 8-amino-6-(3-fluorophenyl)-N,N-dimethyl-5-(1-methyl-1H-1,3-benzodiazol-6-yl)imidazo[1,2-a]pyrazine-2-carboxamide

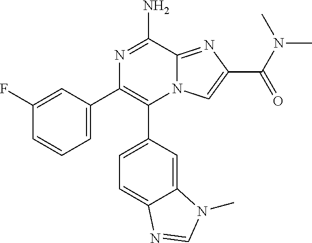 [SMILES: CN(C)C(=O)c1cn2c(-c3ccc4ncn(C)c4c3)c(-c3cccc(F)c3)nc(N)c2n1]

The title compound was synthesized following approach from example 280, substituting dimethylamine (2N in THF) for morpholine at step (B) and substituting (1-methyl-1H-benzimidazol-6-yl)boronic acid for (4-methylquinolin-6-yl)boronic acid, heating at 120° C. for 5 h in step (C) to lead to the title compound (11 mg, 12%) as a yellow solid, hydrochloride salt. ESI-MS: 430.30 [M+H]+. 1H NMR (400 MHz, DMSO-d6) δ 9.42 (s, 1H), 8.17 (s, 1H), 7.90 (s, 2H), 7.89 (d, J=8.5 Hz, 1H), 7.62 (s, 1H), 7.48 (dd, J=8.4, 1.5 Hz, 1H), 7.28-7.17 (m, 2H), 7.14-7.03 (m, 2H), 4.01 (s, 3H), 3.42 (s, 3H), 2.99 (s, 3H).

### Example 286:6-(4-fluorophenyl)-2-(4-methylpiperazine-1-carbonyl)-5-(4-methylquinolin-6-yl)imidazo[1,2-a]pyrazin-8-amine

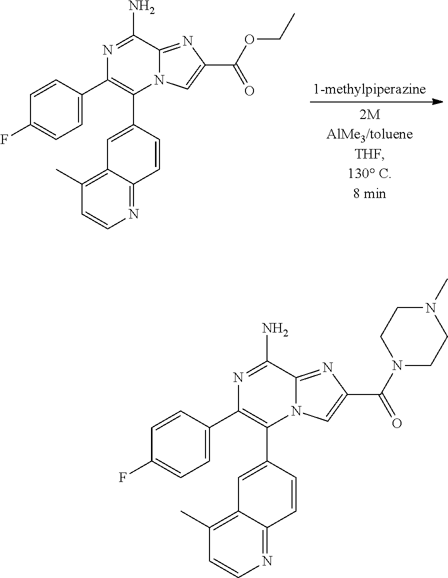 [SMILES: CCOC(=O)c1cn2c(-c3ccc4nccc(C)c4c3)c(-c3ccc(F)cc3)nc(N)c2n1 ; Cc1ccnc2ccc(-c3c(-c4ccc(F)cc4)nc(N)c4nc(C(=O)N5CCN(C)CC5)cn34)cc12]

Ethyl 8-amino-6-(4-fluorophenyl)-5-(4-methylquinolin-6-yl)imidazo[1,2-a]pyrazine-2-carboxylate (example 275) (30 mg, 0.07 mmol) was dissolved in THF and then 1-methylpiperazine (0.01 mL, 0.08 mmol, 1.2 equiv.) was added followed by 2M AIMe₃ in toluene (0.06 mL, 0.07 mmol, 1 equiv.). Reaction was heated under microwave irradiation for 8 min at 130° C. After cooling to r.t. reaction mixture was diluted with DCM, organic layers were washed with water and brine, dried over Na₂SO₄, filtered and concentrated. Remaining residue was purified using flash chromatography (DCM/MeOH 10%) to lead to the title compound (20 mg, 59%) as a yellow solid, hydrochloride salt. ESI-MS: 496.30 [M+H]+. 1H NMR (400 MHz, DMSO-d6) δ 9.06 (d, J=4.8 Hz, 1H), 8.42 (s, 1H), 8.24 (d, J=8.9 Hz, 1H), 7.89 (d, J=8.8 Hz, 1H), 7.85 (s, 1H), 7.77 (s, 1H), 7.41-7.33 (m, 2H), 7.10-7.03 (m, 2H), 5.62-5.39 (m, 2H), 4.65-4.48 (m, 2H), 3.51-3.42 (m, 2H), 3.19-3.00 (m, 2H), 2.80 (s, 3H), 2.73 (s, 3H).

### Example 287: 8-amino-6-(4-fluorophenyl)-5-{3-methylimidazo[1,2-a]pyridin-6-yl}imidazo[1,2-a]pyrazine-2-carboxamide

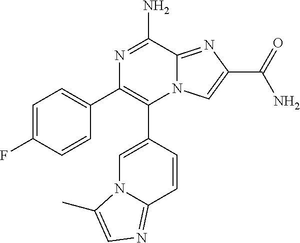 [SMILES: Cc1cnc2ccc(-c3c(-c4ccc(F)cc4)nc(N)c4nc(C(N)=O)cn34)cn12]

The title compound was synthesized following the approach outlined in Procedure G substituting 4-fluorophenylboronic acid for 3-fluorophenylboronic acid in step (a) and substituting (1-methyl-1H-benzimidazol-6-yl)boronic acid for (4-methylquinolin-6-yl)boronic acid in step (f) and substituting Cs₂CO₃ for Na₂CO₃ and heating in mixture dioxane/water 6:1 under microwave irradiation at 130° C. for 1 h to lead to the title compound (9 mg, 10%) as a beige solid, hydrochloride salt. ESI-MS: 402.20 [M+H]+. 1H NMR (400 MHz, DMSO-d6) δ 9.14 (t, J=1.2 Hz, 1H), 8.22 (s, 1H), 8.07 (d, J=1.3 Hz, 1H), 7.98 (d, J=9.2 Hz, 1H), 7.73 (dd, J=9.3, 1.5 Hz, 1H), 7.57 (s, 1H), 7.49-7.42 (m, 3H), 7.14-7.07 (m, 2H), 2.54 (d, J=1.1 Hz, 3H).

### Example 288: 8-amino-6-(4-fluorophenyl)-N-(2-methoxyethyl)-5-(1-methyl-1H-1,3-benzodiazol-6-yl)imidazo[1,2-a]pyrazine-2-carboxamide

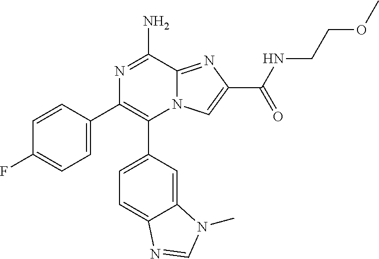 [SMILES: COCCNC(=O)c1cn2c(-c3ccc4ncn(C)c4c3)c(-c3ccc(F)cc3)nc(N)c2n1]

The title compound was synthesized from ethyl 8-amino-5-chloro-6-(4-fluorophenyl)imidazo[1,2-a]pyrazine-2-carboxylate [prepared following the approach outlined in Procedure G, steps (a-d) except substituting 4-fluorophenylboronic acid for 3-fluorophenylboronic acid in step (a)] following approach from example 280, at step (2) substituting 2-methoxyethylenamine for morpholine and in step (3) substituting (1-methyl-1H-benzimidazol-6-yl)boronic acid for (4-methylquinolin-6-yl)boronic acid and substituting Pd(dppf)Cl₂*DCM for Sphos Pd G3 to lead to the title compound (20 mg, 21%) as a brown solid, hydrochloride salt. ESI-MS: 460.2 [M+H]+. 1H NMR (400 MHz, DMSO-d6) δ 9.39 (s, 1H), 8.15 (s, 1H), 8.10 (s, 1H), 7.89 (d, J=8.4 Hz, 1H), 7.76 (s, 1H), 7.45 (dd, J=8.4, 1.5 Hz, 1H), 7.42-7.33 (m, 2H), 7.08 (t, J=8.8 Hz, 2H), 4.01 (s, 3H), 3.45 (d, J=1.9 Hz, 4H), 3.27 (s, 3H).

### Example 289: 8-amino-6-(4-fluorophenyl)-N,N-dimethyl-5-(4-methylquinolin-6-yl)imidazo[1,2-a]pyrazine-2-carboxamide

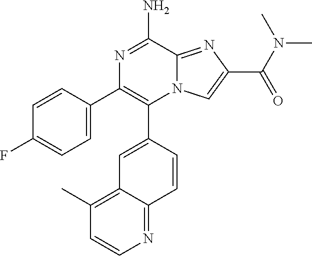 [SMILES: Cc1ccnc2ccc(-c3c(-c4ccc(F)cc4)nc(N)c4nc(C(=O)N(C)C)cn34)cc12]

The title compound was synthesized following approach from example 286, substituting dimethylamine (2N in THF) for 1-methylpiperazine, to lead to the title compound (4.5 mg, 12%) as a yellow solid, hydrochloride salt. ESI-MS: 441.30 [M+H]+. 1H NMR (400 MHz, DMSO-d6) δ 9.07 (d, J=5.1 Hz, 1H), 8.45 (s, 1H), 8.23 (d, J=8.7 Hz, 1H), 7.90 (dd, J=8.7, 1.8 Hz, 1H), 7.82 (s, 1H), 7.78 (d, J=5.2 Hz, 1H), 7.42-7.35 (m, 2H), 7.09 (t, J=8.9 Hz, 2H), 3.38 (s, 3H), 2.98 (s, 3H), 2.74 (s, 3H).

### Example 290:2-[4-(2,4-difluorophenyl)piperazine-1-carbonyl]-6-(4-fluorophenyl)-5-(1-methyl-1H-1,3-benzodiazol-6-yl)imidazo[1,2-a]pyrazin-8-amine

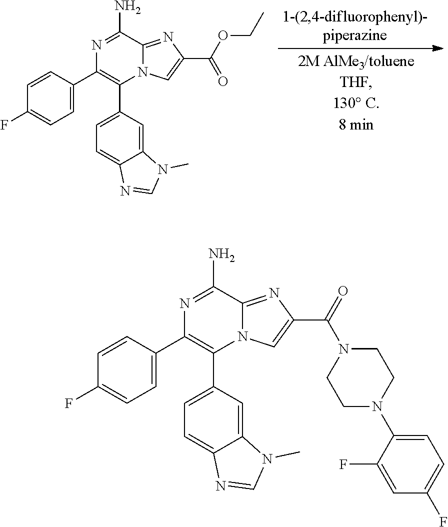 [SMILES: CCOC(=O)c1cn2c(-c3ccc4ncn(C)c4c3)c(-c3ccc(F)cc3)nc(N)c2n1 ; Cn1cnc2ccc(-c3c(-c4ccc(F)cc4)nc(N)c4nc(C(=O)N5CCN(c6ccc(F)cc6F)CC5)cn34)cc21]

Step 1) Ethyl 8-amino-6-(4-fluorophenyl)-5-(1-methyl-1H-1,3-benzodiazol-6-yl)imidazo[1,2-a]pyrazine-2-carboxylate was prepared following the approach outlined in Procedure G, steps (a, b, c, d, f, (excluding step (e))) and substituting 4-fluorophenylboronic acid for 3-fluorophenylboronic acid in step (a) and substituting (1-methyl-1H-benzimidazol-6-yl)boronic acid for (4-methylquinolin-6-yl)boronic acid at step (f) and substituting Pd(amphos)Cl₂ for Sphos Pd G3 and heating at this step for 2 h at 100° C. to lead to the ethyl 8-amino-6-(4-fluorophenyl)-5-(1-methyl-1H-1,3-benzodiazol-6-yl)imidazo[1,2-a]pyrazine-2-carboxylate (880 mg, 68%) as a beige solid. ESI-MS: 431.4 [M+H]+.

Step 2) The title compound was synthesized following approach from example 286, substituting ethyl 8-amino-6-(4-fluorophenyl)-5-(1-methyl-1H-1,3-benzodiazol-6-yl)imidazo[1,2-a]pyrazine-2-carboxylate for ethyl 8-amino-6-(4-fluorophenyl)-5-(4-methylquinolin-6-yl)imidazo[1,2-a]pyrazine-2-carboxylate and substituting 1-(2,4-difluorophenyl)-piperazine for 1-methylpiperazine to lead to the title compound (12 mg, 15%) as a white solid, hydrochloride salt. ESI-MS: 583.3 [M+H]+. 1H NMR (400 MHz, DMSO-d6) δ 9.45 (s, 1H), 8.16 (s, 1H), 7.88 (d, J=8.5 Hz, 1H), 7.71 (s, 1H), 7.45 (dd, J=8.4, 1.3 Hz, 1H), 7.41-7.35 (m 2H), 7.24 (ddd, J=12.1, 8.8, 2.8 Hz, 1H), 7.09 (td, J=9.2, 8.8, 2.3 Hz, 3H), 7.02 (td, J=8.6, 3.2 Hz, 1H), 4.32 (s, 2H), 4.00 (s, 3H), 3.78 (s, 3H), 3.02 (s, 4H).

### Example 291:8-amino-N-({1-[(2,4-difluorophenyl)methyl]piperidin-4-yl}methyl)-6-(4-fluorophenyl)-5-(1-methyl-1H-1,3-benzodiazol-6-yl)imidazo[1,2-a]pyrazine-2-carboxamide

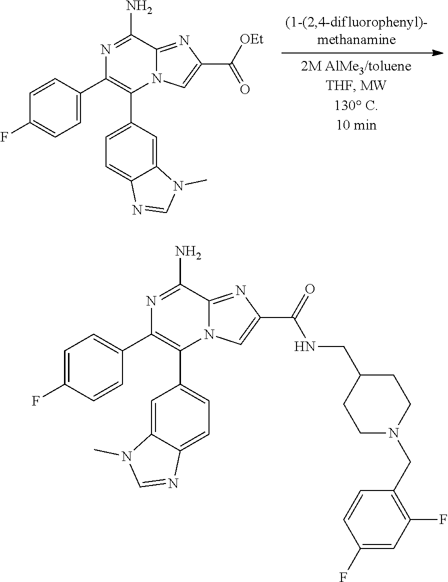

The title compound was synthesized following approach from example 286 substituting ethyl 8-amino-6-(4-fluorophenyl)-5-(1-methyl-1H-1,3-benzodiazol-6-yl)imidazo[1,2-a]pyrazine-2-carboxylate for ethyl 8-amino-6-(4-fluorophenyl)-5-(4-methylquinolin-6-yl)imidazo[1,2-a]pyrazine-2-carboxylate and substituting (1-(2,4-difluorobenzyl)piperidin-4-yl)methanamine for 1-methylpiperazine and heating at 130° C. for 10 min to lead to the title compound (7 mg, 6%) as hydrochloride salt as a yellow solid. ESI-MS: 625.3 [M+H]+. 1H NMR (400 MHz, DMSO-d6) δ 10.64 (s, 1H), 9.44 (s, 1H), 8.48-8.38 (m, 1H), 8.16 (s, 1H), 7.95-7.81 (m, 3H), 7.49-7.34 (m, 4H), 7.28-7.19 (m, 1H), 7.15-7.03 (m, 2H), 4.27 (d, J=4.8 Hz, 2H), 4.03 (s, 4H), 3.41-3.30 (m, 2H), 3.24-3.10 (m, 2H), 3.00-2.87 (m, 2H), 1.87-1.73 (m, 2H), 1.67-1.44 (m, 2H).

### Example 292: 2-[8-amino-6-(4-fluorophenyl)-5-(1-methyl-1H-1,3-benzodiazol-6-yl)imidazo[1,2-a]pyrazin-2-yl]-1-[4-(2,4-difluorophenyl)piperazin-1-yl]ethan-1-one

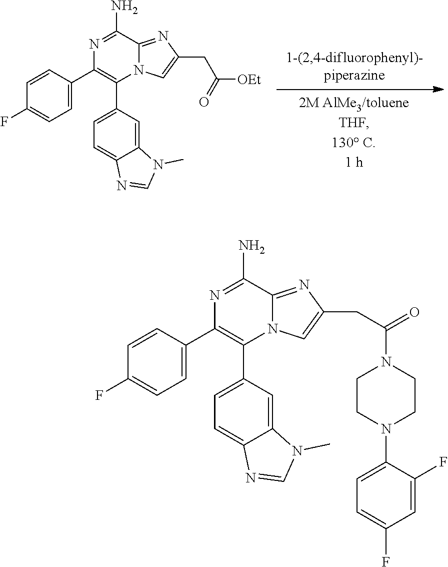 [SMILES: CCOC(=O)Cc1cn2c(-c3ccc4ncn(C)c4c3)c(-c3ccc(F)cc3)nc(N)c2n1 ; Cn1cnc2ccc(-c3c(-c4ccc(F)cc4)nc(N)c4nc(CC(=O)N5CCN(c6ccc(F)cc6F)CC5)cn34)cc21]

Step 1) Ethyl 2-[8-amino-6-(4-fluorophenyl)-5-(1-methyl-1H-1,3-benzodiazol-6-yl)imidazo[1,2-a]pyrazin-2-yl]acetate was synthesized following the approach outlined in Procedure G substituting 4-fluorophenylboronic acid for 3-fluorophenylboronic acid in step (a) and substituting in step (c) ethyl 4-chloroacetoacetate for 3-bromopyruvic acid ethyl ester and heating for 18 h at 100° C. at this step and heating at step (d) for 4 days at 145° C. and excluding the step (e) and substituting (1-methyl-1H-benzimidazol-6-yl)boronic acid for (4-methylquinolin-6-yl)boronic acid in step (f) and substituting Pd(dppf)Cl₂ for Sphos Pd G3 and performing step (f) in dioxane/water 4:1 for 3 h at 130° C. to lead to the title compound (129 mg, 54% for step (f)) as a brown solid. ESI-MS: 445.4 [M+H]+.

Step 2) The title compound was synthesized following the procedure from example 286 substituting ethyl 2-[8-amino-6-(4-fluorophenyl)-5-(1-methyl-1H-1,3-benzodiazol-6-yl)imidazo[1,2-a]pyrazin-2-yl]acetate for ethyl 8-amino-6-(4-fluorophenyl)-5-(4-methylquinolin-6-yl)imidazo[1,2-a]pyrazine-2-carboxylate and substituting 1-(2,4-difluorophenyl)-piperazine (5 equiv.) for 1-methylpiperazine and heating at 130° C. for 1 h to lead to the title compound (28 mg, 14%) as hydrochloride salt as an orange solid. ESI-MS: 597.3 [M+H]+. 1H NMR (400 MHz, DMSO-d6) δ 9.59 (s, 1H), 8.17 (s, 1H), 7.94 (d, J=8.5 Hz, 1H), 7.55-7.50 (m, 2H), 7.47-7.41 (m, 2H), 7.27-7.14 (m, 3H), 7.12-6.99 (m, 2H), 4.01 (s, 3H), 3.97-3.93 (m, 2H), 3.75-3.57 (m, 4H), 3.00-2.86 (m, 4H).

### Example 293: 6-(4-fluorophenyl)-5-(4-methylquinolin-6-yl)-2-(piperazine-1-carbonyl)imidazo[1,2-a]pyrazin-8-amine

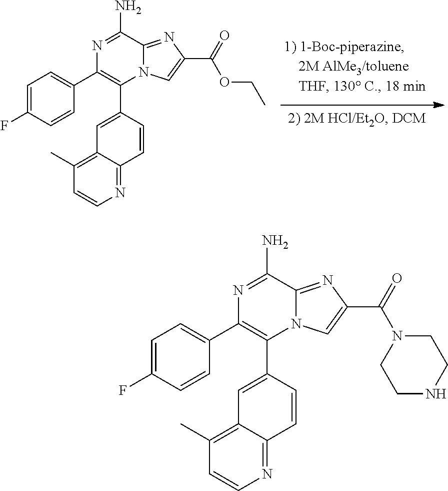 [SMILES: CCOC(=O)c1cn2c(-c3ccc4nccc(C)c4c3)c(-c3ccc(F)cc3)nc(N)c2n1 ; Cc1ccnc2ccc(-c3c(-c4ccc(F)cc4)nc(N)c4nc(C(=O)N5CCNCC5)cn34)cc12]

Step 1) Tert-butyl 4-[8-amino-6-(4-fluorophenyl)-5-(4-methylquinolin-6-yl)imidazo[1,2-a]pyrazine-2-carbonyl]piperazine-1-carboxylate was prepared following approach outlined in synthesis of example 286 substituting 1-Boc-piperazine for 1-methylpiperazine and heating at this step for 18 min to lead to the product as a beige solid (18 mg, 34%). ESI-MS: 582.5 [M+H]+.

Step 2) The title compound was obtained from tert-butyl 4-[8-amino-6-(4-fluorophenyl)-5-(4-methylquinolin-6-yl)imidazo[1,2-a]pyrazine-2-carbonyl]piperazine-1-carboxylate according to procedure B, step (f. 2) to lead to the 6-(4-fluorophenyl)-5-(4-methylquinolin-6-yl)-2-(piperazine-1-carbonyl)imidazo[1,2-a]pyrazin-8-amine as hydrochloride salt (16 mg, quant.) as a yellow solid. ESI-MS: 482.3 [M+H]+. 1H NMR (400 MHz, DMSO-d6) δ 9.27 (s, 2H), 9.07 (d, J=5.1 Hz, 1H), 8.43 (s, 1H), 8.25 (d, J=8.7 Hz, 1H), 7.92-7.88 (m, 1H), 7.86 (s, 1H), 7.78 (s, 1H), 7.40-7.35 (m, 2H), 7.07 (t, J=8.9 Hz, 2H), 4.47 (s, 2H), 3.83 (s, 2H), 3.18 (s, 4H), 2.74 (s, 3H).

### Example 294:6-(4-fluoro-3-methylphenyl)-5-(1-methyl-1H-1,3-benzodiazol-6-yl)imidazo[1,2-a]pyrazin-8-amine

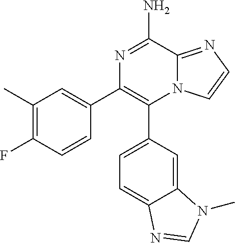 [SMILES: Cc1cc(-c2nc(N)c3nccn3c2-c2ccc3ncn(C)c3c2)ccc1F]

The title compound was synthesized following the approach outlined in Procedure F substituting at step (a) (1-methyl-1H-benzimidazol-6-yl)boronic acid for (8-fluoroquinolin-6-yl)boronic acid and substituting Pd(dppf)Cl₂ for Pd(amphos)Cl₂ in step (a) and substituting N-chlorosuccinimide for N-bromosuccinimide in step (b) and substituting chloroacetaldehyde for 2-bromo-1,1-dimethoxyethane in step (c) and performing this step in acetonitrile/dioxane 1:1 mixture and heating under microwave irradiation at 110° C. for 1 h at step (c) and substituting acetonitrile for dioxane at step (d) and substituting 4-fluoro-3-methylphenylboronic acid for 3-pyridineboronic acid pinacol ester in step (e) to afford the title compound as a hydrochloride salt (white solid, 11 mg, 11%). ESI-MS: 373.2 [M+H]+. 1H NMR (400 MHz, DMSO-d6) δ 9.47 (s, 1H), 8.99 (s, 1H), 8.14 (s, 1H), 7.94-7.86 (m, 2H), 7.64 (d, J=1.2 Hz, 1H), 7.48-7.39 (m, 2H), 7.14-7.07 (m, 1H), 7.05-6.98 (m, 1H), 4.01 (s, 3H), 2.15 (s, 3H).

### Example 295: 6-(6-fluoropyridin-3-yl)-5-(1-methyl-1H-1,3-benzodiazol-6-yl)imidazo[1,2-a]pyrazin-8-amine

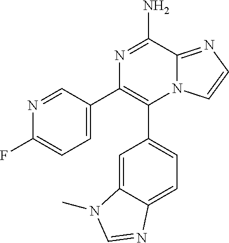 [SMILES: Cn1cnc2ccc(-c3c(-c4ccc(F)nc4)nc(N)c4nccn34)cc21]

The title compound was synthesized following the approach outlined in Procedure F substituting at step (a) (1-methyl-1H-benzimidazol-6-yl)boronic acid for (8-fluoroquinolin-6-yl)boronic acid and substituting Pd(dppf)Cl₂ for Pd(amphos)Cl₂ in step (a) and substituting N-chlorosuccinimide for N-bromosuccinimide in step (b) and substituting chloroacetaldehyde for 2-bromo-1,1-dimethoxyethane in step (c) and performing this step in acetonitrile/dioxane 1:1 mixture and heating under microwave irradiation at 110° C. for 1 h at step (c) and substituting acetonitrile for dioxane at step (d) and substituting (6-fluoropyridin-3-yl)boronic acid for 3-pyridineboronic acid pinacol ester in step (e) to afford the title compound as a hydrochloride salt (white solid, 7 mg, 12%). ESI-MS: 360.1 [M+H]+. 1H NMR (400 MHz, DMSO-d6) δ 9.49 (s, 1H), 8.48 (s, 1H), 8.21-8.08 (m, 2H), 7.99-7.84 (m, 3H), 7.63 (d, J=1.2 Hz, 1H), 7.48 (d, J=8.5 Hz, 1H), 7.15 (dd, J=8.6, 2.5 Hz, 1H), 4.01 (s, 3H).

### Example 296: 6-(4-fluorophenyl)-5-(1-methyl-1H-1,3-benzodiazol-6-yl)-2-(4-methylpiperazine-1-carbonyl)imidazo[1,2-a]pyrazin-8-amine

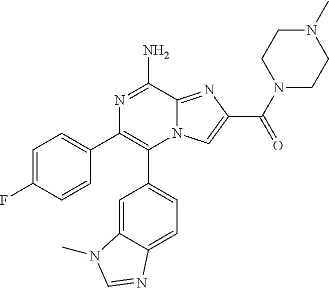 [SMILES: CN1CCN(C(=O)c2cn3c(-c4ccc5ncn(C)c5c4)c(-c4ccc(F)cc4)nc(N)c3n2)CC1]

The title compound was synthesized following approach from example 286, substituting ethyl 8-amino-6-(4-fluorophenyl)-5-(1-methyl-1H-1,3-benzodiazol-6-yl)imidazo[1,2-a]pyrazine-2-carboxylate for ethyl 8-amino-6-(4-fluorophenyl)-5-(4-methylquinolin-6-yl)imidazo[1,2-a]pyrazine-2-carboxylate and heating at 130° C. for 8 min to lead to the title compound (7 mg, 20%) as a beige solid, as a hydrochloride salt. ESI-MS: 485.3 [M+H]+. 1H NMR (400 MHz, DMSO-d6) δ 11.08 (s, 1H), 9.42 (s, 1H), 8.14 (s, 1H), 7.88 (d, J=8.4 Hz, 1H), 7.69 (s, 1H), 7.44 (d, J=8.5 Hz, 1H), 7.40-7.34 (m, 2H), 7.07 (t, J=8.9 Hz, 2H), 5.55 (s, 2H), 4.55 (d, J=13.1 Hz, 1H), 3.99 (s, 3H), 3.47 (d, J=12.2 Hz, 4H), 2.80 (d, J=4.3 Hz, 3H).

### Example 297: 6-(6-fluoropyridin-2-yl)-5-(1-methyl-1H-1,3-benzodiazol-6-yl)imidazo[1,2-a]pyrazin-8-amine

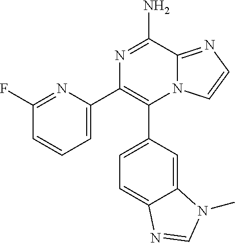 [SMILES: Cn1cnc2ccc(-c3c(-c4cccc(F)n4)nc(N)c4nccn34)cc21]

The title compound was synthesized following the approach outlined in Procedure F substituting at step (a) (1-methyl-1H-benzimidazol-6-yl)boronic acid for (8-fluoroquinolin-6-yl)boronic acid and substituting Pd(dppf)Cl₂ for Pd(amphos)Cl₂ in step (a) and substituting N-chlorosuccinimide for N-bromosuccinimide in step (b) and substituting chloroacetaldehyde for 2-bromo-1,1-dimethoxyethane in step (c) and performing this step in acetonitrile/dioxane 1:1 mixture and heating under microwave irradiation at 110° C. for 1 h at step (c) and substituting acetonitrile for dioxane at step (d) and substituting (6-fluoropyridin-2-yl)boronic acid for 3-pyridineboronic acid pinacol ester in step (e) to afford the title compound as a hydrochloride salt (white solid, 4 mg, 6%). ESI-MS: 360.1 [M+H]+. 1H NMR (400 MHz, Methanol-d4) δ 9.58 (s, 1H), 8.32 (dd, J=1.5, 0.8 Hz, 1H), 8.12 (dd, J=8.6, 0.7 Hz, 1H), 7.85 (d, J=1.2 Hz, 1H), 7.81 (dd, J=8.5, 1.5 Hz, 1H), 7.74 (q, J=8.0 Hz, 1H), 7.60 (d, J=1.3 Hz, 1H), 7.12 (dd, J=8.2, 2.5 Hz, 1H), 6.91 (dd, J=7.7, 2.3 Hz, 1H).

### Example 298: 2-[8-amino-6-(4-fluorophenyl)-5-(1-methyl-1H-1,3-benzodiazol-6-yl)imidazo[1,2-a]pyrazin-2-yl]acetamide

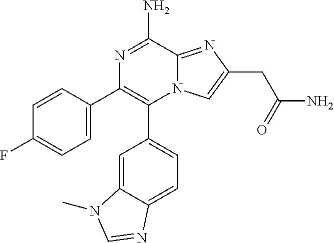 [SMILES: Cn1cnc2ccc(-c3c(-c4ccc(F)cc4)nc(N)c4nc(CC(N)=O)cn34)cc21]

The title compound was synthesized following the approach outlined in Procedure G, steps (af) substituting 4-fluorophenylboronic acid for 3-fluorophenylboronic acid in step (a) and substituting in step (c) ethyl 4-chloroacetoacetate for 3-bromopyruvic acid ethyl ester and heating for 18 h at 100° C. at this step and heating at step (d) for 4 days at 145° C. and substituting (1-methyl-1H-benzimidazol-6-yl)boronic acid for (4-methylquinolin-6-yl)boronic acid in step (f) and substituting Pd(dppf)Cl₂ for Sphos Pd G3 and performing step (f) in dioxane/water 4:1 for 3 h at 130° C. to lead to the title compound (8 mg, 6%) as a white solid. ESI-MS: 416.2 [M+H]+. 1H NMR (400 MHz, DMSO-d6) δ 8.01 (s, 1H), 7.87 (d, J=8.4 Hz, 1H), 7.51-7.38 (m, 4H), 7.19-7.08 (m, 2H), 7.03 (s, 1H), 3.95 (s, 3H), 3.59 (s, 2H).

### Example 299: [8-amino-6-(4-fluorophenyl)-5-(1-methyl-1H-1,3-benzodiazol-6-yl)imidazo[1,2-a]pyrazin-2-yl]methanol

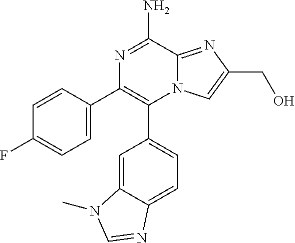 [SMILES: Cn1cnc2ccc(-c3c(-c4ccc(F)cc4)nc(N)c4nc(CO)cn34)cc21]

The title compound was prepared according the procedure from example 138, starting from ethyl 8-amino-6-(4-fluorophenyl)-5-(1-methyl-1H-1,3-benzodiazol-6-yl)imidazo[1,2-a]pyrazine-2-carboxylate (example 300) to lead to 8-amino-6-(4-fluorophenyl)-5-(1-methyl-1H-1,3-benzodiazol-6-yl)imidazo[1,2-a]pyrazin-2-yl]methanol as a yellow solid (4.5 mg, 10%). ESI-MS: 389.2 [M+H]+. 1H NMR (300 MHz, Methanol-d4) δ 8.22 (s, 1H), 7.76 (d, J=8.3 Hz, 1H), 7.63-7.59 (m, 1H), 7.40-7.34 (m, 3H), 7.30 (dd, J=8.4, 1.6 Hz, 1H), 6.96-6.86 (m, 2H), 4.71 (s, 2H), 4.59 (s, 1H), 3.86 (s, 3H).

### Example 300: ethyl 8-amino-6-(4-fluorophenyl)-5-(1-methyl-1H-1,3-benzodiazol-6-yl)imidazo[1,2-a]pyrazine-2-carboxylate

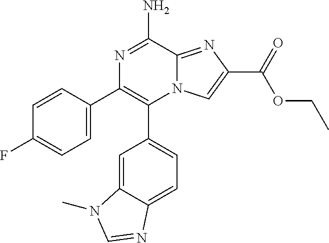 [SMILES: CCOC(=O)c1cn2c(-c3ccc4ncn(C)c4c3)c(-c3ccc(F)cc3)nc(N)c2n1]

The title compound was prepared following the approach outlined in Procedure G, steps [a, b, c, d, f, (excluding step (e))] and substituting 4-fluorophenylboronic acid for 3-fluorophenylboronic acid in step (a) and substituting (1-methyl-1H-benzimidazol-6-yl)boronic acid for (4-methylquinolin-6-yl)boronic acid at step (f) and substituting Pd(amphos)Cl₂ for Sphos Pd G3 and heating at this step for 2 h at 100° C. to lead to the title compound (880 mg, 68%) as a yellow solid as a hydrochloride salt. ESI-MS: 431.7 [M+H]+. 1H NMR (400 MHz, Methanol-d4) δ 8.25 (s, 1H), 7.82 (s, 1H), 7.79 (dd, J=8.4, 0.7 Hz, 1H), 7.64 (dd, J=1.6, 0.7 Hz, 1H), 7.42-7.35 (m, 2H), 7.33 (dd, J=8.4, 1.6 Hz, 1H), 6.94-6.87 (m, 2H), 4.38 (q, J=7.1 Hz, 2H), 3.87 (s, 3H), 1.36 (t, J=7.1 Hz, 3H).

### Example 301: 6-(4-fluorophenyl)-5-(4-methylquinolin-6-yl)-2-(morpholine-4-carbonyl)imidazo[1,2-a]pyrazin-8-amine

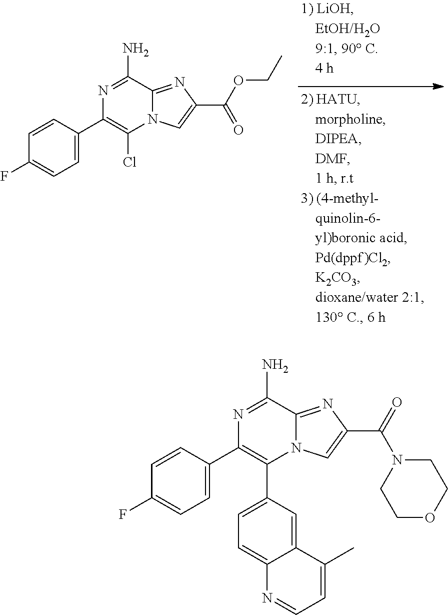 [SMILES: CCOC(=O)c1cn2c(Cl)c(-c3ccc(F)cc3)nc(N)c2n1 ; Cc1ccnc2ccc(-c3c(-c4ccc(F)cc4)nc(N)c4nc(C(=O)N5CCOCC5)cn34)cc12]

The title compound was prepared following approach outlined in synthesis of example 280, except in step (1) ethyl 8-amino-5-chloro-6-(4-fluorophenyl)imidazo[1,2-a]pyrazine-2-carboxylate [prepared according to Procedure G, steps (a-d) substituting 4-fluorophenylboronic acid for 3-fluorophenylboronic acid in step (a)] was substituted in place of ethyl 8-amino-5-chloro-6-(3-fluorophenyl)imidazo[1,2-a]pyrazine-2-carboxylate and in step (3) (4-methylquinolin-6-yl)boronic acid (example 129) was substituted for (1-methyl-1H-benzimidazol-6-yl)boronic acid and Pd(dppf)Cl₂ was substituted for Pd SPhos G3 and reaction was heated for 130° C. for 6 h to lead to the title compound (5 mg, 13%) as a yellow solid. ESI-MS: 483.2 [M+H]+. 1H NMR (300 MHz, Deuterium Oxide) 9.01 (d, J=5.7 Hz, 1H), 8.52 (d, J=1.7 Hz, 1H), 8.26 (d, J=8.9 Hz, 1H), 8.14-7.93 (m, 3H), 7.42 (dd, J=8.8, 5.3 Hz, 2H), 7.13-7.01 (m, 2H), 4.08 (s, 2H), 3.84 (d, J=10.8 Hz, 6H), 2.89 (d, J=0.8 Hz, 3H).

### Example 302: 5-(1-methyl-1H-1,3-benzodiazol-6-yl)-6-(pyridin-4-yl)imidazo[1,2-a]pyrazin-8-amine

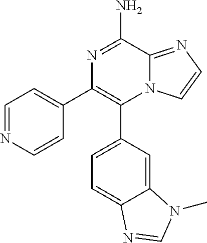 [SMILES: Cn1cnc2ccc(-c3c(-c4ccncc4)nc(N)c4nccn34)cc21]

The title compound was synthesized following the approach outlined in Procedure F substituting at step (a) (1-methyl-1H-benzimidazol-6-yl)boronic acid for (8-fluoroquinolin-6-yl)boronic acid and substituting Pd(dppf)Cl₂ for Pd(amphos)Cl₂ in step (a) and substituting N-chlorosuccinimide for N-bromosuccinimide in step (b) and substituting chloroacetaldehyde for 2-bromo-1,1-dimethoxyethane in step (c) and performing this step in acetonitrile/dioxane 1:1 mixture and heating under microwave irradiation at 110° C. for 1 h at step (c) and substituting acetonitrile for dioxane at step (d) and substituting (pyridin-4-yl)boronic acid for 3-pyridineboronic acid pinacol ester in step (e) to afford the title compound as a hydrochloride salt (yellow solid, 4 mg, 10%). ESI-MS: 342.1 [M+H]+. 1H NMR (400 MHz, DMSO-d6) δ 9.42 (s, 1H), 8.74-8.55 (m, 2H), 8.19 (s, 1H), 7.94 (d, J=8.4 Hz, 1H), 7.74 (d, J=1.3 Hz, 1H), 7.69 (d, J=5.9 Hz, 2H), 7.59-7.43 (m 2H), 4.00 (s. 3H).

### Example 303: 5-(1-ethyl-1H-1,3-benzodiazol-6-yl)-6-(4-fluorophenyl)imidazo[1,2-a]pyrazin-8-amine

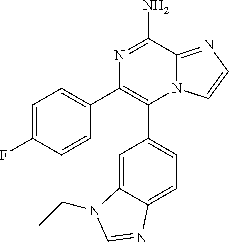 [SMILES: CCn1cnc2ccc(-c3c(-c4ccc(F)cc4)nc(N)c4nccn34)cc21]

The title compound was synthesized following the approach outlined in Procedure D, substituting 6-chloro-5-(4-fluorophenyl)pyrazin-2-amine [Procedure B, step (b) substituting 4-fluorophenylboronic acid for phenylboronic acid] for 6-chloro-5-phenylpyrazin-2-amine in step (a) and substituting (1-ethyl-1H-benzimidazol-6-yl)boronic acid for 5-(tetramethyl-1,3,2-dioxaborolan-2-yl)-1H-indazole in step (d) and using the conditions A described in Procedure D heating at 130° C. for 3 h to lead to the title compound (37 mg, 31%) as a hydrochloride salt as an white solid. ESI-MS: 373.5 [M+H]+. 1H NMR (400 MHz, DMSO-d6) δ 9.56 (s, 1H), 9.05 (s, 2H), 8.14 (s, 1H), 7.97-7.89 (m, 2H), 7.70 (d, J=1.2 Hz, 1H), 7.52 (dd, J=8.5, 1.4 Hz, 1H), 7.44-7.36 (m, 2H), 7.19-7.11 (m, 2H), 4.43 (q, J=7.8, 7.2 Hz, 2H), 1.42 (t, J=7.3 Hz, 3H).

### Example 304: 1-[8-amino-6-(4-fluorophenyl)-5-(4-methylquinolin-6-yl)imidazo[1,2-a]pyrazine-2-carbonyl]-4-methylpiperidin-4-ol

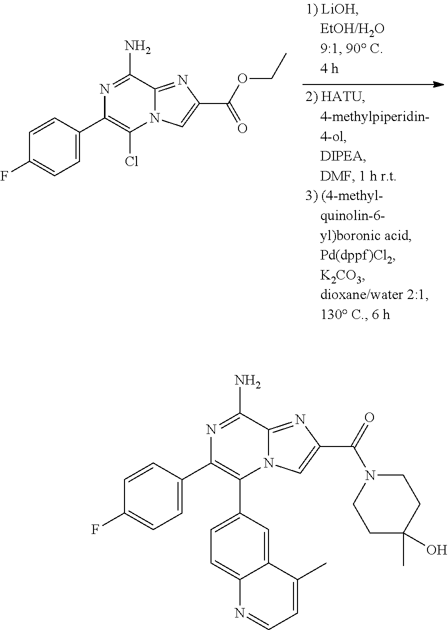 [SMILES: CCOC(=O)c1cn2c(Cl)c(-c3ccc(F)cc3)nc(N)c2n1 ; Cc1ccnc2ccc(-c3c(-c4ccc(F)cc4)nc(N)c4nc(C(=O)N5CCC(C)(O)CC5)cn34)cc12]

The title compound was prepared following approach outlined in synthesis of example 280, except in step (1) ethyl 8-amino-5-chloro-6-(4-fluorophenyl)imidazo[1,2-a]pyrazine-2-carboxylate [prepared according to Procedure G, steps (a-d) substituting 4-fluorophenylboronic acid for 3-fluorophenylboronic acid in step (a)] was substituted in place of ethyl 8-amino-5-chloro-6-(3-fluorophenyl)imidazo[1,2-a]pyrazine-2-carboxylate and in step (2) 4-methylpiperidin-4-ol was substituted in place of morpholine and reaction was performed for 1 h and in step (3(4-methylquinolin-6-yl)boronic acid (example 129) was substituted for (1-methyl-1H-benzimidazol-6-yl)boronic acid and Pd(dppf)Cl₂ was substituted for Pd SPhos G3 and reaction was heated for 130° C. for 6 h to lead to the title compound (13 mg, 18%) as a brown solid. ESI-MS: 511.3 [M+H]+. 1H NMR (400 MHz, DMSO-d6) δ 8.94 (d, J=4.8 Hz, 1H), 8.31 (d, J=1.9 Hz, 1H), 8.11 (d, J=8.6 Hz, 1H), 7.82 (dd, J=8.8, 1.8 Hz, 1H), 7.72 (s, 2H), 7.61 (d, J=4.8 Hz, 1H), 7.44-7.29 (m, 2H), 7.05 (t, J=8.9 Hz, 2H), 4.42 (s, 2H), 4.05 (d, J=12.6 Hz, 3H), 3.20 (t, J=11.8 Hz, 2H), 2.65 (s, 3H), 1.47 (d, J=28.8 Hz, 4H), 1.15 (s, 3H).

Part 2: Activity

Examples 1 to 304 (see synthesis part above for the structures and names of these 304 compounds) were tested for their antagonistic activity at the rat A2A receptor (endogenously expressed in PC12 cells, which were used in the assay). The antagonistic activity was determined by measuring the effect of each compound on agonist-induced cAMP production using the assay based on time-resolved fluorescence resonance energy transfer (TR-FRET).

General reference as regards the cells and background can be made to Gao et al., “Novel short-acting A2A adenosine receptor agonists for coronary vasodilation: inverse relationship be-tween affinity and duration of action of A21A agonists”, J. Pharmacol. Exp. Ther., 298, 209.

More specifically, the assay to test each of examples 1 to 304 was performed as follows: The cells were suspended in HBSS buffer (Invitrogen) complemented with 20 mM HEPES (pH 7.4), with 0.1% BSA and 100 μM Rolipram (a phosphodiesterase-4 inhibitor to block the degradation of cAMP), then distributed in microplates at a density of 2.10³ cells/well (in a 384 well plate) in the presence of either (i) HBSS (basal control) with 0.2% DMSO, (ii) the test compound, i.e. each of examples 1 to 304, or (iii) the reference antagonist ZM 241385.

Thereafter, the reference adenosine receptor agonist NECA (e.g. CAS 35920-39-9, Calbiochem) was added at a final concentration of 43 nM (concentration corresponding to EC₈₀). For basal control measurements, separate assay wells did not contain NECA.

Following 30 min incubation at room temperature, the cells were lysed and the detection mix was added (standard reagents used according to a standard protocol; LANCE™ cAMP 384 Kit, PerkinElmer).

After 60 min at room temperature, the fluorescence transfer was measured at λex=340 nm and λem=665 nm using a microplate reader according to a standard protocol (Envison, Perkin Elmer).

For test compounds % of normalized vehicle control was calculated for each data point and plotted against test compound concentration:

$$y = 100 - 100 * \frac{Sample - Low control}{Vehicle control - Low control}$$

Sample—mean fluorescence intensity of tested compound

Low control—mean fluorescence intensity of NECA 0.043 μM

Vehicle control—mean fluorescence intensity of DMSO 0.2%

EC50, Hill slope and efficacy parameters were determined by fitting a variable-slope sigmoidal function.

For each of example 1 to 304, the apparent dissociation constant (KB) was calculated using the modified Cheng Prusoff equation:

$$K B = \frac{E C 50}{1 + ( \frac{A}{E C 50 A} )}$$

where A=concentration of reference agonist in the assay, and EC50 Å=EC50 value of the reference agonist.

The standard reference antagonist used was ZM 241385, which was tested in each experiment at several concentrations to generate a concentration-response curve from which its EC50 value was calculated.

Each of examples 1 to 304 exhibited A2A receptor antagonist activity (KB<10 μM). Thus, examples 1 to 304 as described herein are potent A2A receptor antagonists.

Preferred embodiments of aspects B1 to B5 of the present invention relate to:
1. A compound of formula (I)

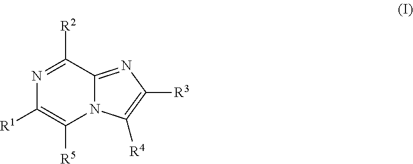

or a salt, stereoisomer, tautomer or N-oxide thereof,
wherein
R¹ is selected from the group consisting of a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said Nand/or S-atoms are independently oxidized or non-oxidized, and wherein each substitutable carbon or heteroatom in the aforementioned cyclic or bicyclic moieties is independently unsubstituted or substituted with one or more, same or different substituents R⁶;
R² is selected from the group consisting of halogen and N(R^{12a})(R^{12b});
R³ is selected from the group consisting of
   (i) H, halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁸;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
R⁴ is H;
R⁵ is selected from the group consisting of a 5- to 9-membered saturated, partially unsaturated or fully unsaturated carbocyclic or heterocyclic ring and a 9- to 12-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁷;
R⁶ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁷;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
R⁷ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R¹⁰;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
R⁸ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁹;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
R⁹ is selected from the group consisting of
   (i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R¹⁰;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
R¹⁰ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
R¹¹, R¹², R^{12a}, R^{12b} are independently selected from the group consisting of
   (i) H, C₁-C₆-alkyl, C₂-C₆-alkenyl, and C₂-C₆-alkynyl, wherein each substitutable carbon atom in the aforementioned moieties is independently unsubstituted or substituted with one or more, same or different substituents R¹³;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁴;
R¹³ is selected from the group consisting of
   (i) halogen, CN, NO₂, C₁-C₆-alkyl, C₁—C-haloalkyl, C₂-C₆-alkenyl, C₂-C₆-haloalkenyl, C₂-C₆-alkynyl, C₂-C₆-haloalkynyl, N(R^{15a})(R^{15b}), C(═O)NR^{15a}R^{15b}, S(═O)ₙNR^{15a}R^{15b}, OR¹⁵ and S(═O)ₙR¹⁵;
   (ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁴;
R¹⁴ is selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C(═O)R¹⁶, C(═O)OR¹⁵, C(═O)SR¹⁵, C(═O)N(R^{15a})(R^{15b}), OR¹⁵, S(═O)ₙR¹⁵, S(═O)ₙN(R^{15a})(R^{15b}), S(═O)ₘOR¹⁵, N(R^{15a})(R^{15b}), N(R¹⁵)C(═O)R¹⁶, N(R¹⁵)C(═O)OR¹⁵, N(R¹⁵)C(═O)N(R^{15a})(R^{15b}), N(R¹⁵)S(═O)ₙ(R¹⁵), N(R¹⁵)S(═O)ₘN(R^{15a})(R^{15b}), and N(R¹⁵)S(═O)ₘOR¹⁵;
R¹⁵, R^{15a}, R^{15b}, R¹⁶ are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
R¹⁷ is selected from the group consisting of
   (i) halogen, CN, NO₂, C₁-C₄-alkyl, C₂-C₄-alkenyl, and C₂-C₄-alkynyl, wherein each substitutable carbon or hetero-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
R¹⁸ is selected from the group consisting of halogen, N(R^{20a})(R^{20b}), and OR²⁰;
R¹⁹, R²⁰, R^{20a}, R^{20b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
and wherein
n is 0, 1 or 2; and
m is 1 or 2.
2. The compound according to 1, wherein R² is NH₂ and wherein all other substituents have the meaning as defined in 1.
3. The compound according to 1 or 2, wherein R¹ is selected from the group consisting of a 5- to 6-membered fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl; and wherein all other substituents have the meaning as defined in 1.
4. The compound according to any one of 1, 2 or 3, wherein R⁵ is selected from the group consisting of a 5- to 6-membered fully unsaturated carbocyclic or heterocyclic ring and a 9- to 10-membered fully unsaturated carbobicyclic or heterobicyclic ring, wherein said heterocyclic or heterobicyclic ring comprises one or more N-atoms, and wherein said N-atoms are independently oxidized or non-oxidized, and wherein each substitutable carbon or heteroatom in the aforementioned cyclic moieties is independently substituted with one or more, same or different substituents R¹⁷, and wherein each substitutable carbon or heteroatom in the aforementioned bicyclic moieties is independently unsubstituted or substituted with one or more, same or different substituents R¹⁷ and wherein all other substituents have the meaning as defined in 1.
5. The compound according to any one of 1, 2, 3 or 4, wherein R⁵ has the formula (S1)

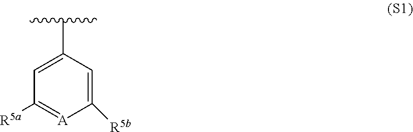

and wherein
   A is N or CR⁵;
R^{5a}, R^{5b}, R^{5c} are independently selected from the group consisting of
   (i) H, halogen, CN, NO₂, C₁-C₄-alkyl, C₂-C₄-alkenyl, and C₂-C₄-alkynyl, wherein each substitutable carbon or hetero-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
      with the proviso that at least one of R^{5a}, R^{5b}, R^{5c} is not H;
or
R^{5a} is selected from the group consisting of
   (i) H, halogen, CN, NO₂, C₁-C₄-alkyl, C₂-C₄-alkenyl, and C₂-C₄-alkynyl, wherein each substitutable carbon or hetero-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
   and
R^{5b} and R^{5c} together with the atoms to which they are attached form a 5- to 6-membered fully unsaturated carbocyclic or heterocyclic ring, wherein said heterocyclic ring comprises one or more N-atoms, and wherein said N-atoms are independently oxidized or non-oxidized, and wherein each substitutable carbon or heteroatom in the aforementioned cyclic moieties is independently unsubstituted or substituted with one or more, same or different substituents selected from the group consisting of
   (i) H, halogen, CN, NO₂, C₁-C₄-alkyl, C₂-C₄-alkenyl, and C₂-C₄-alkynyl, wherein each substitutable carbon or hetero-atom in the aforementioned moieties is unsubstituted or substituted with one or more, same or different substituents R¹⁸;
   (ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
   and wherein all other substituents have the meaning as defined in 1.
6. The compound according to any one of 1, 2, 3, 4 or 5, wherein R⁵ is selected from the group consisting of a 6-membered fully unsaturated carbocyclic or heterocyclic ring and a 9- to 10-membered fully unsaturated heterobicyclic ring, wherein said heterocyclic or heterobicyclic ring comprises one or more N-atoms, and wherein said N-atoms are independently oxidized or non-oxidized, and wherein each substitutable carbon or heteroatom in the aforementioned cyclic moieties is independently substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, OR²⁰, and N(R^{20a})(R^{20b}), and wherein each substitutable carbon or heteroatom in the aforementioned bicyclic moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, OR²⁰, and N(R^{20a})(R^{20b}); and wherein all other substituents have the meaning as defined in 1.
7. The compound according to any one of 1, 2, 3, 4, 5, or 6, wherein R⁸ is selected from the group consisting of
   (i) halogen, CN, NO₂, C₁-C₆-alkyl, C₂-C₆-alkenyl, and C₂-C₆-alkynyl, wherein each substitutable carbon or hetero-atom in the aforementioned moieties is unsubstituted or substituted with one or more, same or different substituents R⁹;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²; and
   wherein R⁹ is selected from the group consisting of
   (i) halogen, CN, NO₂, C₁-C₆-alkyl, C₂-C₆-alkenyl, and C₂-C₆-alkynyl,
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹².
8. The compound according to any one of 1, 2, 3, 4, 5, 6, or 7, wherein R³ is selected from the group consisting of
   (i) H, halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
   and wherein all other substituents have the meaning as defined in 1.
9. The compound according to 1, wherein R¹ is selected from the group consisting of a 5- to 6-membered fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, and wherein R² is NH₂, and wherein R³ is selected from the group consisting of
   (i) H, halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
   (ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
   and wherein R⁵ is selected from the group consisting of a 6-membered fully unsaturated carbocyclic or heterocyclic ring and a 9- to 10-membered fully unsaturated heterobicyclic ring, wherein said heterocyclic or heterobicyclic ring comprises one or more N-atoms, and wherein said N-atoms are independently oxidized or non-oxidized, and wherein each substitutable carbon or heteroatom in the aforementioned cyclic moieties is independently substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, OR²⁰, and N(R^{20a})(R^{20b}), and wherein each substitutable carbon or heteroatom in the aforementioned bicyclic moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, OR²⁰, and N(R^{20a})(R^{20b}) and wherein all other substituents have the meaning as defined in 1.
10. The compound according to any one of 1, 2, 3, 4, 5, 6, 7, 8 or 9, wherein said compound is selected from the group consisting of 4-{8-amino-6-phenylimidazo[1,2-a]pyrazin-5-yl}-2-chlorophenol; 4-[8-amino-6-(furan-2-yl)imidazo[1,2-a]pyrazin-5-yl]-2-chlorophenol; 5-(2,6-dimethylpyridin-4-yl)-6-phenylimidazo[1,2-a]pyrazin-8-amine; 5-[2-methyl-6-(trifluoromethyl)pyridin-4-yl]-6-phenylimidazo[1,2-a]pyrazin-8-amine; 4-[8-bromo-6-(furan-2-yl)imidazo[1,2-a]pyrazin-5-yl]-2-chlorophenol; 4-[8-amino-6-(4-fluorophenyl)imidazo[1,2-a]pyrazin-5-yl]-2-chlorophenol; 6-(4-fluorophenyl)-5-[2-methyl-6-(trifluoromethyl)pyridin-4-yl]imidazo[1,2-a]pyrazin-8-amine; 5-(2,6-dimethylpyridin-4-yl)-6-(4-fluorophenyl)imidazo[1,2-a]pyrazin-8-amine; 6-(3-fluorophenyl)-5-[2-methyl-6-(trifluoromethyl)pyridin-4-yl]imidazo[1,2-a]pyrazin-8-amine; 3-{8-amino-5-[2-methyl-6-(trifluoromethyl)pyridin-4-yl]imidazo[1,2-a]pyrazin-6-yl}benzonitrile; 3-[8-amino-5-(3-chloro-4-hydroxyphenyl)imidazo[1,2-a]pyrazin-6-yl]benzonitrile; 4-[8-amino-6-(3-fluorophenyl)imidazo[1,2-a]pyrazin-5-yl]-2-chlorophenol; 4-[8-amino-2-(3-nitrophenyl)-6-phenylimidazo[1,2-a]pyrazin-5-yl]-2-chlorophenol; 5-(1H-indazol-5-yl)-6-phenylimidazo[1,2-a]pyrazin-8-amine; 4-{8-amino-6-phenylimidazo[1,2-a]pyrazin-5-yl}-2,6-dichlorophenol; 4-{8-amino-2-cyclohexyl-6-phenylimidazo[1,2-a]pyrazin-5-yl}-2-chlorophenol; 4-{8-amino-6-phenylimidazo[1,2-a]pyrazin-5-yl}-2-bromo-6-chlorophenol; 4-{8-amino-6-[4-(trifluoromethyl)phenyl]imidazo[1,2-a]pyrazin-5-yl}-2-chlorophenol; 3-[8-amino-5-(3-chloro-4-hydroxyphenyl)-6-phenylimidazo[1,2-a]pyrazin-2-yl]benzonitrile; 4-[8-amino-5-(3-chloro-4-hydroxyphenyl)imidazo[1,2-a]pyrazin-6-yl]benzonitrile; 4-{8-amino-6-phenylimidazo[1,2-a]pyrazin-5-yl}-N-methylpyridin-2-amine; 4-{8-amino-2,6-diphenylimidazo[1,2-a]pyrazin-5-yl}-2-chlorophenol; 4-[8-amino-6-(3-fluorophenyl)imidazo[1,2-a]pyrazin-5-yl]phenol; 4-[8-amino-6-(3-fluorophenyl)imidazo[1,2-a]pyrazin-5-yl]-N-methylpyridin-2-amine; 8-amino-5-(3-chloro-4-hydroxyphenyl)-6-phenylimidazo[1,2-a]pyrazine-2-carboxamide; 4-[8-amino-6-(4-fluorophenyl)imidazo[1,2-a]pyrazin-5-yl]-N-methylpyridin-2-amine; and 6-(3-fluorophenyl)-5-(quinolin-6-yl)imidazo[1,2-a]pyrazin-8-amine.
11. A pharmaceutical composition comprising a pharmaceutically effective amount of the compound according to any one of 1 to 10 and optionally a pharmaceutically acceptable carrier, diluent or excipient.
12. A compound according to any one of 1 to 10 and a pharmaceutical composition according to 11 for use in medicine.
13. A compound for use according to 12 or a pharmaceutical composition for use according to 11 or 12, wherein said compound or pharmaceutical composition is for use in the treatment of a disease selected from the group consisting of cancer, Parkinson's disease, Huntington's disease, Alzheimer's disease, psychosis, stroke, extra pyramidal syndrome (in particular dystonia, akathisia, pseudoparkinsonism and tardive dyskinesia), attention deficit disorder (ADD), attention deficit hyperactivity disorder (ADHD), amyotrophic lateral sclerosis, cirrhosis, fibrosis, fatty liver, addictive behavior, dermal fibrosis (in particular dermal fibrosis in scleroderma), sleep disorders, AIDS, autoimmune diseases, infections, atherosclerosis and ischemia-reperfusion injury.
14. A compound for use according to 12 or a pharmaceutical composition for use according to 11 or 12, wherein said compound or pharmaceutical composition is for use in the treatment of cancer, and wherein at least one further anti-neoplastic agent is preferably coadministered with said compound and/or comprised in said pharmaceutical composition.
15. A compound for use according to 14 or a pharmaceutical composition for use according to 14, wherein said anti-neoplastic agent is selected from the group consisting of a chemotherapeutic agent and a checkpoint inhibitor, wherein said checkpoint inhibitor is preferably an antibody selected from the group consisting of an anti-PD-1, anti-PDL1 anti-CTLA-4, anti-IDO, anti-KIR, anti-TIM-3 and anti-LAG3 antibody.

1. A compound of formula (I): 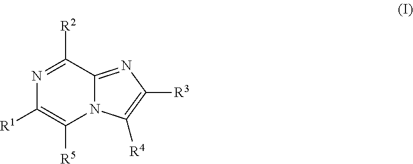
or a salt, stereoisomer, tautomer, isotopologue, or N-oxide thereof,
wherein
R¹ is selected from the group consisting of a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁶;
R² is NH₂;
R³ is selected from the group consisting of
(i) H, halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁸;
(ii) C(═O)R²⁵, C(═O)OR²⁶, C(═O)SR²⁶, C(═O)N(R^{26a})(R^{26b}), OR²⁶, S(═O)ₙR²⁶, S(═O)ₙN(R^{26a})(R^{26b}), S(═O)ₘOR²⁶, N(R^{26a})(R^{26b}), N(R²⁶)C(═O)R²⁵, N(R²⁶)C(═O)OR²⁶, N(R²⁶)C(═O)N(R^{26a})(R^{26b}), N(R²⁶)S(═O)ₙ(R²⁶), N(R²⁶)S(═O)ₘN(R^{26a})(R^{26b}), and N(R²⁶)S(═O)ₘOR²⁶;
R⁴ is H;
R⁵ is selected from the group consisting of a 5- to 9-membered saturated, partially unsaturated or fully unsaturated carbocyclic or heterocyclic ring and a 9- to 12-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁷;
R⁶ is selected from the group consisting of
(i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁷;
(ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
and/or two R⁶ present on one C-atom together form ═O;
R⁷ is selected from the group consisting of
(i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R¹⁰;
(ii) C(═O)R²¹, C(═O)OR²², C(═O)SR²², C(═O)N(R^{22a})(R^{22b}), OR²², S(═O)ₙR²², S(═O)ₙN(R^{22a})(R^{22b}), S(═O)ₘOR²², N(R^{22a})(R^{22b}), N(R²²)C(═O)R²¹, N(R²²)C(═O)OR²², N(R²²)C(═O)N(R^{22a})(R^{22b}), N(R²²)S(═O)ₙ(R²²), N(R²²)S(═O)ₘN(R^{22a})(R^{22b}), and N(R²²)S(═O)ₘOR²²;
R⁸ is selected from the group consisting of
(i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁹;
(ii) C(═O)R²⁷, C(═O)OR²⁸, C(═O)SR²⁸, C(═O)N(R^{28a})(R^{28b}), OR²⁸, S(═O)ₙR²⁸, S(═O)ₙN(R^{28a})(R^{28b}), S(═O)ₘOR²⁸, N(R^{28a})(R^{28b}), N(R²⁸)C(═O)R²⁷, N(R²⁸)C(═O)OR²⁸, N(R²⁸)C(═O)N(R^{28a})(R^{28b}), N(R²⁸)S(═O)ₙ(R²⁸), N(R²⁸)S(═O)ₘN(R^{28a})(R^{28b}), and N(R²⁸)S(═O)ₘOR²;
R⁹ is selected from the group consisting of
(i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R²⁹;
(ii) C(═O)R³⁰, C(═O)OR³¹, C(═O)SR³¹, C(═O)N(R^{31a})(R^{31b}), OR³¹, S(═O)ₙR³¹, S(═O)ₙN(R^{31a})(R^{31b}) S(═O)ₘOR³¹, N(R^{31a})(R^{31b}) N(R³¹)C(═O)R³⁰, N(R³¹)C(═O)OR³¹, N(R³¹)C(═O)N(R^{31a})(R^{31b}) N(R³¹)S(═O)ₙ(R³¹), N(R³¹)S(═O)ₘN(R^{31a})(R^{31b}) and N(R³¹)S(═O)ₘOR³¹;
R¹⁰ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
R¹¹, R¹², R^{12a}, and R^{12b} are independently selected from the group consisting of
(i) H, C₁-C₆-alkyl, C₂-C₆-alkenyl, and C₂-C₆-alkynyl, wherein each substitutable carbon atom in the aforementioned moieties is independently unsubstituted or substituted with one or more, same or different substituents R¹³;
(ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁴;
R¹³ is selected from the group consisting of
(i) halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, N(R^{15a})(R^{15b}), C(═O)NR^{15a}R^{15b}, S(═O)ₙNR^{15a}R^{15b}, OR¹⁵ and S(═O)ₙR¹⁵;
(ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁴;
R¹⁴ is selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C(═O)R¹⁶, C(═O)OR¹⁵, C(═O)SR¹⁵, C(═O)N(R^{15a})(R^{15b}), OR¹⁵, S(═O)ₙR¹⁵, S(═O)ₙN(R^{15a})(R^{15b}), S(═O)ₘOR¹⁵, N(R^{15a})(R^{15b}), N(R¹⁵)C(═O)R¹⁶, N(R¹⁵)C(═O)OR¹⁵, N(R¹⁵)C(═O)N(R^{15a})(R^{15b}), N(R¹⁵)S(═O)ₙ(R¹⁵), N(R¹⁵)S(═O)ₘN(R^{15a})(R^{15b}), and N(R¹⁵)S(═O)ₘOR¹⁵;
R¹⁵, R^{15a}, R^{15b}, and R¹⁶ are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
R¹⁷ is selected from the group consisting of
(i) halogen, CN, NO₂, C₁-C₄-alkyl, C₂-C₄-alkenyl, C₂-C₄-alkynyl, and a 3- to 9-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R¹¹;
(ii) C(═O)R¹⁹, C(═O)OR², C(═O)SR²⁰, C(═O)N(R^{26a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
and/or two R¹⁷ present on one C-atom together form ═O;
R¹⁸ is selected from the group consisting of
(i) halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
(ii) C(═O)R²³, C(═O)OR²⁴, C(═O)SR²⁴, C(═O)N(R^{24a})(R^{24b}), OR²⁴, S(═O)ₙR²⁴, S(═O)ₙN(R^{24a})(R^{24b}), S(═O)ₘOR²⁴, N(R^{24a})(R^{24b}), N(R²⁴)C(═O)R²³, N(R²⁴)C(═O)OR²⁴, N(R²⁴)C(═O)N(R^{24a})(R^{24b}), N(R²⁴)S(═O)ₙ(R²⁴), N(R²⁴)S(═O)ₘN(R^{24a})(R^{24b}), and N(R²⁴)S(═O)ₘOR²⁴;
R¹⁹, R²⁰, R^{20a}, and R^{20b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
R²¹, R²², R^{22a}, and R^{22b} are independently selected from the group consisting of
(i) H, C₁-C₆-alkyl, C₂-C₆-alkenyl, and C₂-C₆-alkynyl, wherein each substitutable carbon atom in the aforementioned moieties is independently unsubstituted or substituted with one or more, same or different substituents R¹³;
(ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁴;
R²³, R²⁴, R^{24a}, and R^{24b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
R²⁵, R²⁶, R^{26a}, and R^{26b} are independently selected from the group consisting of H, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R³²;
R²⁷, R²⁸, R^{28a}, and R^{28b} are independently selected from the group consisting of H, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R³³;
R²⁹ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(═O)R³⁴, C(═O)OR³⁵, C(═O)SR³⁵, C(═O)N(R^{35a})(R^{35b}), OR³⁵, S(═O)ₙR³⁵, S(═O)ₙN(R^{35a})(R^{35b}), S(═O)ₘOR³⁵, N(R^{35a})(R^{35b}), N(R³⁵)C(═O)R³⁴, N(R³⁵)C(═O)OR³⁵, N(R³⁵)C(═O)N(R^{35a})(R^{35b}), N(R³⁵)S(═O)ₙ(R³⁵), N(R³⁵)S(═O)ₘN(R^{35a})(R^{35b}), and N(R³⁵)S(═O)ₘOR³⁵;
R³⁰, R³¹, R^{31a}, and R^{31b} are independently selected from the group consisting of H, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R³⁷;
R³² is selected from the group consisting of
(i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R³⁸;
(ii) C(═O)R³⁹, C(═O)OR⁴⁰, C(═O)SR⁴⁰, C(═O)N(R^{40a})(R^{40b}), OR⁴⁰, S(═O)ₙR⁴⁰, S(═O)ₙN(R^{40a})(R^{40b}), S(═O)ₘOR⁴⁰, N(R^{40a})(R^{40b}), N(R⁴⁰)C(═O)R³⁹, N(R⁴⁰)C(═O)OR⁴⁰, N(R⁴⁰)C(═O)N(R^{40a})(R^{40b}), N(R⁴⁰)S(═O)ₙ(R⁴⁰), N(R⁴⁰)S(═O)ₘN(R^{40a})(R^{40b}), and N(R⁴⁰)S(═O)ₘOR⁴⁰;
R³³ is selected from the group consisting of
(i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁴¹;
(ii) C(═O)R⁴², C(═O)OR⁴³, C(═O)SR⁴³, C(═O)N(R^{43a})(R^{43b}), OR⁴³, S(═O)ₙR⁴³, S(═O)ₙN(R^{43a})(R^{43b}), S(═O)ₘOR⁴³, N(R^{43a})(R^{43b}), N(R⁴³)C(═O)R⁴², N(R⁴³)C(═O)OR⁴³, N(R⁴³)C(═O)N(R^{43a})(R^{43b}), N(R⁴³)S(═O)ₙ(R⁴³), N(R⁴³)S(═O)ₘN(R^{43a})(R^{43b}), and N(R⁴³)S(═O)ₘOR⁴³;
R³⁴, R³⁵, R^{35a}, and R^{35b} are independently selected from the group consisting of
(i) H, C₁-C₆-alkyl, C₂-C₆-alkenyl, and C₂-C₆-alkynyl, wherein each substitutable carbon atom in the aforementioned moieties is independently unsubstituted or substituted with one or more, same or different substituents R³⁶;
(ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁵²;
R³⁶ is selected from the group consisting of
(i) halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, N(R^{53a})(R^{53b}), C(═O)NR^{53a}R^{53b}, S(═O)ₙNR^{53a}R^{53b}, OR⁵³ and S(═O)ₙR⁵³;
(ii) a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁵²;
R³⁷ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, OH, O(C₁-C₄-alkyl), NH₂, NH(C₁-C₄-alkyl), and N(C₁-C₄-alkyl)(C₁-C₄-alkyl);
R³⁸ is selected from the group consisting of
(i) halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁴⁴;
(ii) C(═O)R⁴⁵, C(═O)OR⁴⁶, C(═O)SR⁴⁶, C(═O)N(R^{46a})(R^{46b}), OR⁴⁶, S(═O)ₙR⁴⁶, S(═O)ₙN(R^{46a})(R^{46b}), S(═O)ₘOR⁴⁶, N(R^{46a})(R^{46b}), N(R⁴⁶)C(═O)R⁴⁵, N(R⁴⁶)C(═O)OR⁴⁶, N(R⁴⁶)C(═O)N(R^{46a})(R^{46b}), N(R⁴⁶)S(═O)ₙ(R⁴⁶), N(R⁴⁶)S(═O)ₘN(R^{46a})(R^{46b}), and N(R⁴⁶)S(═O)ₘOR⁴⁶;
R³⁹, R⁴⁰, R^{40a}, and R^{40b} are independently selected from the group consisting of H, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁴⁷;
R⁴¹ is selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, OH, O(C₁-C₄-alkyl), NH₂, NH(C₁-C₄-alkyl), and N(C₁-C₄-alkyl)(C₁-C₄-alkyl);
R⁴², R⁴³, R^{43a}, and R^{43b} are independently selected from the group consisting of H, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁴⁸;
R⁴⁴ is selected from the group consisting of halogen, CN, NO₂, C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁴⁹;
R⁴⁵, R⁴⁶, R^{46a}, and R^{46b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
R⁴⁷ is selected from the group consisting of halogen, CN, NO₂, OH, O(C₁-C₄-alkyl), NH₂, NH(C₁-C₄-alkyl), and N(C₁-C₄-alkyl)(C₁-C₄-alkyl), C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R¹¹;
R⁴⁸ is selected from the group consisting of halogen, CN, NO₂, OH, O(C₁-C₄-alkyl), NH₂, NH(C₁-C₄-alkyl), and N(C₁-C₄-alkyl)(C₁-C₄-alkyl), C₁-C₆-alkyl, C₂-C₆-alkenyl, C₂-C₆-alkynyl, a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or hetero-atom in the aforementioned moieties is unsubstituted or substituted with one or more, same or different substituents R⁵¹;
R⁴⁹, R⁵⁰, and R⁵¹ are independently selected from the group consisting of halogen, CN, NO₂, C₁-C₆-alkyl, C₁-C₆-haloalkyl, C₂-C₆-alkenyl, C₂-C₆-haloalkenyl, C₂-C₆-alkynyl, C₂-C₆-haloalkynyl, OH, O(C₁-C₄-alkyl), NH₂, NH(C₁-C₄-alkyl), and N(C₁-C₄-alkyl)(C₁-C₄-alkyl);
R⁵² is selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C(═O)R⁵⁴, C(═O)OR⁵³, C(═O)SR⁵³, C(═O)N(R^{53a})(R^{53b}), OR⁵³, S(═O)ₙR⁵³, S(═O)ₙN(R^{53a})(R^{53b}), S(═O)ₘOR⁵³, N(R^{53a})(R^{53b}), N(R⁵³)C(═O)R⁵⁴, N(R⁵³)C(═O)OR⁵³, N(R⁵³)C(═O)N(R^{53a})(R^{53b}), N(R⁵³)S(═O)ₙ(R⁵³), N(R⁵³)S(═O)ₘN(R^{53a})(R^{53b}), and N(R⁵³)S(═O)ₘOR⁵³;
R⁵³, R^{53a}, R^{53b}, and R⁵⁴ are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
and wherein
n is 0, 1 or 2; and
m is for 2.

2. The compound according to claim 1, wherein R¹ is selected from the group consisting of a 3- to 9-membered saturated, partially unsaturated or fully unsaturated carbocyclic or heterocyclic ring and a 6- to 14-membered saturated, partially unsaturated or fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁶;
wherein R⁶ is selected from the group consisting of
(i) halogen, CN, NO₂, C₁-C₆-alkyl, C₂-C₆-alkenyl, and C₂-C₆-alkynyl, wherein each substitutable carbon-atom in the aforementioned moieties is unsubstituted or substituted with one or more, same or different substituents R⁷;
(ii) C(═O)R¹¹, C(═O)OR¹², C(═O)SR¹², C(═O)N(R^{12a})(R^{12b}), OR¹², S(═O)ₙR¹², S(═O)ₙN(R^{12a})(R^{12b}), S(═O)ₘOR¹², N(R^{12a})(R^{12b}), N(R¹²)C(═O)R¹¹, N(R¹²)C(═O)OR¹², N(R¹²)C(═O)N(R^{12a})(R^{12b}), N(R¹²)S(═O)ₙ(R¹²), N(R¹²)S(═O)ₘN(R^{12a})(R^{12b}), and N(R¹²)S(═O)ₘOR¹²;
and/or two R⁶ present on one C-atom together form ═O;
wherein R⁷ is selected from the group consisting of
(i) halogen, CN, NO₂, C₁-C₆-alkyl, C₂-C₆-alkenyl, and C₂-C₆-alkynyl,
(ii) C(═O)R²¹, C(═O)OR²², C(═O)SR²², C(═O)N(R^{22a})(R^{22b}), OR²², S(═O)ₙR²², S(═O)ₙN(R^{22a})(R^{22b}), S(═O)ₘOR²², N(R^{22a})(R^{22b}), N(R²²)C(═O)R²¹, N(R²²)C(═O)OR²², N(R²²)C(═O)N(R^{22a})(R^{22b}), N(R²²)S(═O)ₙ(R²²), N(R²²)S(═O)ₘN(R^{22a})(R^{22b}), and N(R²²)S(═O)ₘOR²²;
wherein R¹¹, R¹², R^{12a}, R^{12b}, R²¹, R²², R^{22a}, and R^{22b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
and wherein all other substituents have the meaning as defined in claim 1.

3. The compound according to claim 1, wherein R¹ is selected from the group consisting of a 5- to 6-membered fully unsaturated carbocyclic or heterocyclic ring and a 9- to 10-membered fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁶;
wherein R⁶ is selected from the group consisting of
(i) halogen, CN, NO₂, C₁-C₃-alkyl, C₂-C₃-alkenyl, and C₂-C₃-alkynyl, wherein each substitutable carbon-atom in the aforementioned moieties is unsubstituted or substituted with one or more, same or different substituents R⁷;
(ii) C(═O)R¹¹, C(═O)OR¹², C(═O)N(R^{12a})(R^{12b}), OR¹², N(R^{12a})(R^{12b}), N(R¹²)C(═O)R¹¹, N(R¹²)C(═O)OR¹², and N(R¹²)C(═O)N(R^{12a})(R^{12b});
and/or two R⁶ present on one C-atom together form ═O;
wherein R⁷ is selected from the group consisting of
(i) halogen, CN, NO₂, C₁-C₆-alkyl, C₂-C₆-alkenyl, and C₂-C₆-alkynyl,
(ii) C(═O)R²¹, C(═O)OR²², C(═O)N(R^{22a})(R^{22b}), OR²², N(R^{22a})(R^{22b}), N(R²²)C(═O)R²¹, N(R²²)C(═O)OR²², and N(R²²)C(═O)N(R^{22a})(R^{22b});
wherein R¹¹, R¹², R^{12a}, R^{12b}, R²¹, R²², R^{22a}, and R^{22b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl; and
wherein all other substituents have the meaning as defined in claim 1.

4. The compound according to claim 1, wherein R¹ is a 5- to 6-membered fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, OH, O(C₁-C₄-alkyl), NH₂, NH(C₁-C₄-alkyl), and N(C₁-C₄-alkyl)(C₁-C₄-alkyl); and wherein all other substituents have the meaning as defined in claim 1.

5. The compound according to claim 1, wherein R⁵ is selected from the group consisting of a 5- to 6-membered partially unsaturated or fully unsaturated carbocyclic or heterocyclic ring and a 9- to 10-membered partially unsaturated or fully unsaturated carbobicyclic or heterobicyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁷, and wherein all other substituents have the meaning as defined in claim 1.

6. The compound according to claim 1, wherein R⁵ is selected from the group consisting of a 5- to 6-membered partially unsaturated or fully unsaturated carbocyclic or heterocyclic ring and a 9- to 10-membered partially unsaturated or fully unsaturated carbobicyclic or heterobicyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁷;
wherein R¹⁷ is selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, C(═O)R¹⁹, C(═O)OR²⁰, C(═O)N(R^{20a})(R^{20b}), OR²⁰, N(R^{20a})(R^{20b}), N(R²⁰)C(═O)R¹⁹, N(R²⁰)C(═O)OR²⁰, and N(R²⁰)C(═O)N(R^{20a})(R^{20b});
and/or two R¹⁷ present on one C-atom together form ═O;
wherein R¹⁹, R²⁰, R^{20a}, and R^{20b} are independently selected from the group consisting of H, C₁-C₂-alkyl, and C₁-C₂-haloalkyl; and
wherein all other substituents have the meaning as defined in claim 1.

7. The compound according to claim 1, wherein R⁵ has the formula (S1): 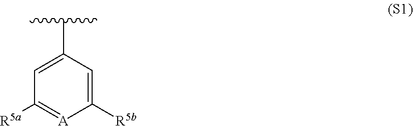
and wherein
A is N or CR^{5c};
R^{5a}, R^{5b}, and R^{5c} are independently selected from the group consisting of
(i) H, halogen, CN, NO₂, C₁-C₄-alkyl, C₂-C₄-alkenyl, and C₂-C₄-alkynyl, wherein each substitutable carbon-atom in the aforementioned moieties is unsubstituted or substituted with one or more, same or different substituents R¹⁸;
(ii) C(═O)R¹⁹, C(═O)OR², C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
with the proviso that at least one of R^{5a}, R^{5b}, R^{5c} is not H;
or
R^{5a} is selected from the group consisting of
(i) H, halogen, CN, NO₂, C₁-C₄-alkyl, C₂-C₄-alkenyl, and C₂-C₄-alkynyl, wherein each substitutable carbon-atom in the aforementioned moieties is unsubstituted or substituted with one or more, same or different substituents R¹⁸;
(ii) C(═O)R¹⁹, C(═O)OR²⁰, C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
and
R^{5b} and R^{5c} together with the atoms to which they are attached form a 5- to 6-membered partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is independently unsubstituted or substituted with one or more, same or different substituents selected from the group consisting of
(i) H, halogen, CN, NO₂, C₁-C₄-alkyl, C₂-C₄-alkenyl, and C₂-C₄-alkynyl, wherein each substitutable carbon-atom in the aforementioned moieties is unsubstituted or substituted with one or more, same or different substituents R¹⁸;
(ii) C(═O)R¹⁹, C(═O)OR², C(═O)SR²⁰, C(═O)N(R^{20a})(R^{20b}), OR²⁰, S(═O)ₙR²⁰, S(═O)ₙN(R^{20a})(R^{20b}), S(═O)ₘOR²⁰, N(R^{20a})(R^{20b}), N(R²⁰)C(═O)R¹⁹, N(R²⁰)C(═O)OR²⁰, N(R²⁰)C(═O)N(R^{20a})(R^{20b}), N(R²⁰)S(═O)ₙ(R²⁰), N(R²⁰)S(═O)ₘN(R^{20a})(R^{20b}), and N(R²⁰)S(═O)ₘOR²⁰;
and wherein all other substituents have the meaning as defined in claim 1.

8. The compound according to claim 1, wherein R⁵ is selected from the group consisting of a 6-membered fully unsaturated carbocyclic or heterocyclic ring and a 9- to 10-membered fully unsaturated heterobicyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic moieties is independently substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, C(═O)R¹⁹, C(═O)OR²⁰, C(═O)N(R^{20a})(R^{20b}), OR²⁰, N(R^{20a})(R^{20b}), N(R²⁰)C(═O)R¹⁹, N(R²⁰)C(═O)OR²⁰, and N(R²⁰)C(═O)N(R^{20a})(R^{20b}); and wherein each substitutable carbon or heteroatom in the aforementioned bicyclic moieties is independently unsubstituted or substituted with one or more, same or different substituents selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, and C₂-C₄-haloalkynyl, C(═O)R¹⁹, C(═O)OR²⁰, C(═O)N(R^{20a})(R^{20b}), OR²⁰, N(R^{20a})(R^{20b}), N(R²⁰)C(═O)R¹⁹, N(R²⁰)C(═O)OR²⁰, and N(R²⁰)C(═O)N(R^{20a})(R^{20b});
wherein R¹⁹, R²⁰, R^{20a}, and R^{20b} are independently selected from the group consisting of H, C₁-C₂-alkyl, and C₁-C₂-haloalkyl,
and wherein all other substituents have the meaning as defined in claim 1.

9. The compound according to claim 1, wherein R³ is selected from the group consisting of
(i) H, C₁-C₆-alkyl, a 3- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁸;
(ii) C(═O)R²⁵, C(═O)OR²⁶, C(═O)N(R^{26a})(R^{26b}), OR²⁶, N(R^{26a})(R^{26b}), N(R²⁶)C(═O)R²⁵, N(R²⁶)C(═O)OR²⁶, and N(R²⁶)C(═O)N(R^{26a})(R^{26b});
wherein R⁸ is selected from the group consisting of
(i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁹;
(ii) C(═O)R²⁷, C(═O)OR²⁸, C(═O)N(R^{28a})(R^{28b}), OR²⁸, N(R^{28a})(R^{28b}), N(R²⁸)C(═O)R²⁷, N(R²⁸)C(═O)OR²⁸, and N(R²⁸)C(═O)N(R^{28a})(R^{28b});
wherein R⁹ is selected from the group consisting of
(i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R²⁹;
(ii) C(═O)R³⁰, C(═O)OR³¹, C(═O)N(R^{31a})(R^{31b}) OR³¹, N(R^{31a})(R^{31b}), N(R³¹)C(═O)R³⁰, N(R³¹)C(═O)OR³¹, N(R³¹)C(═O)N(R^{31a})(R^{31b});
wherein R²⁵, R²⁶, R^{26a}, and R^{26b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³²;
wherein R²⁷, R²⁸, R^{28a}, and R^{28b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³³;
wherein R²⁹ is selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein R³⁰, R³¹, R^{31a}, and R^{31b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³⁷;
wherein R³² is selected from the group consisting of
(i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³⁸;
(ii) C(═O)R³⁹, C(═O)OR⁴⁰, C(═O)N(R^{40a})(R^{40b}), OR⁴⁰, N(R^{40a})(R^{40b}), N(R⁴⁰)C(═O)R³⁹, N(R⁴⁰)C(═O)OR⁴⁰, N(R⁴⁰)C(═O)N(R^{40a})(R^{40b});
wherein R³³ is selected from the group consisting of
(i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴¹;
(ii) C(═O)R⁴², C(═O)OR⁴³, C(═O)N(R^{43a})(R^{43b}), OR⁴³, N(R^{43a})(R^{43b}), N(R⁴³)C(═O)R⁴², N(R⁴³)C(═O)OR⁴³, N(R⁴³)C(═O)N(R^{43a})(R^{43b});
wherein R³⁷ is selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein R³⁸ is selected from the group consisting of
(i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁴;
(ii) C(═O)R⁴⁵, C(═O)OR⁴⁶, C(═O)N(R^{46a})(R^{46b}), OR⁴⁶, N(R^{46a})(R^{46b}), N(R⁴⁶)C(═O)R⁴⁵, N(R⁴⁶)C(═O)OR⁴⁶, N(R⁴⁶)C(═O)N(R^{46a})(R^{46b});
wherein R³⁹, R⁴⁰, R^{40a}, and R^{40b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁷;
wherein R⁴¹ is selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein R⁴², R⁴³, R^{43a}, and R^{43b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁸;
wherein R⁴⁴ is selected from the group consisting of halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴;
wherein R⁴⁵, R⁴⁶, R^{46a}, and R^{46b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
wherein R⁴⁷ is selected from the group consisting of halogen, CN, NO₂, OH, O(C₁-C₄-alkyl), NH₂, NH(C₁-C₄-alkyl), and N(C₁-C₄-alkyl)(C₁-C₄-alkyl), C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R¹¹;
wherein R⁴⁸ is selected from the group consisting of halogen, CN, NO₂, OH, O(C₁-C₄-alkyl), NH₂, NH(C₁-C₄-alkyl), and N(C₁-C₄-alkyl)(C₁-C₄-alkyl), C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁵¹;
wherein R⁴⁹, R⁵⁰, and R⁵¹ are independently selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
and wherein all other substituents have the meaning as defined in claim 1.

10. The compound according to claim 9, wherein R³ is selected from the group consisting of
(i) (L¹)_{y}-X¹,
(ii) (L¹)_{y}-X¹-(L²)_{y}-X², and
(iii) (L¹)_{y}-X¹-(L²)_{y}-X²-(L³)_{y}-X³; and
(iv) (L¹)_{y}-X¹-(L²)_{y}-X²-(L³)_{y}-X³-(L⁴)_{y}-X⁴
wherein X¹, X², X³, and X⁴ are independently selected from the group consisting of H, C₁-C₆-alkyl, a 3- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein L¹, L², L³, and L⁴ are independently selected from the group consisting of C(═O), C(═O)O, C(═O)N(R^{a}), O, N(R^{a}), N(R^{a})C(═O),
wherein R^{a} is selected from the group consisting of H, C₁-C₄-alkyl, and C₁-C₄-haloalkyl, wherein y is 0 or 1,
and wherein all other substituents have the meaning as defined in claim 1.

11. The compound according to claim 1, wherein R¹ is selected from the group consisting of a 5- to 6-membered fully unsaturated carbocyclic or heterocyclic ring and a 9- to 10-membered fully unsaturated carbobicyclic or heterobicyclic ring, wherein said heterocyclic or heterobicyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R⁶;
wherein R⁶ is selected from the group consisting of
(i) halogen, CN, NO₂, C₁-C₃-alkyl, C₂-C₃-alkenyl, and C₂-C₃-alkynyl, wherein each substitutable carbon-atom in the aforementioned moieties is unsubstituted or substituted with one or more, same or different substituents R⁷;
(ii) C(═O)R¹¹, C(═O)OR¹², C(═O)N(R^{12a})(R^{12b}), OR¹², N(R^{12a})(R^{12b}), N(R¹²)C(═O)R¹¹, N(R¹²)C(═O)OR¹², and N(R¹²)C(═O)N(R^{12a})(R^{12b});
and/or two R⁶ present on one C-atom together form ═O;
wherein R⁷ is selected from the group consisting of
(i) halogen, CN, NO₂, C₁-C₆-alkyl, C₂-C₆-alkenyl, and C₂-C₆-alkynyl,
(ii) C(═O)R²¹, C(═O)OR²², C(═O)N(R^{22a})(R^{22b}), OR²², N(R^{22a})(R^{22b}), N(R²²)C(═O)R²¹, N(R²²)C(═O)OR²², and N(R²²)C(═O)N(R^{22a})(R^{22b});
wherein R¹¹, R¹², R^{12a}, R^{12b}, R²¹, R²², R^{22a}, and R^{22b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
wherein R³ is selected from the group consisting of
(i) H, C₁-C₆-alkyl, a 3- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁸;
(ii) C(═O)R²⁵, C(═O)OR²⁶, C(═O)N(R^{26a})(R^{26b}), OR²⁶, N(R^{26a})(R^{26b}), N(R²⁶)C(═O)R²⁵, N(R²⁶)C(═O)OR²⁶, and N(R²⁶)C(═O)N(R^{26a})(R^{26b});
wherein R⁸ is selected from the group consisting of
(i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁹;
(ii) C(═O)R²⁷, C(═O)OR²⁸, C(═O)N(R^{28a})(R^{28b}), OR²⁸, N(R^{28a})(R^{28b}), N(R²⁸)C(═O)R²⁷, N(R²⁸)C(═O)OR²⁸, and N(R²⁸)C(═O)N(R^{28a})(R^{28b});
wherein R⁹ is selected from the group consisting of
(i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R²⁹;
(ii) C(═O)R³⁰, C(═O)OR³¹, C(═O)N(R^{31a})(R^{31b}) OR³¹, N(R^{31a})(R^{31b}), N(R³¹)C(═O)R³⁰, N(R³¹)C(═O)OR³¹, N(R³¹)C(═O)N(R^{31a})(R^{31b});
wherein R²⁵, R²⁶, R^{26a}, and R^{26b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³²;
wherein R²⁷, R²⁸, R^{28a}, and R^{28b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³³;
wherein R²⁹ is selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein R³⁰, R³¹, R^{31a}, and R^{31b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³⁷;
wherein R³² is selected from the group consisting of
(i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R³⁸;
(ii) C(═O)R³⁹, C(═O)OR⁴⁰, C(═O)N(R^{40a})(R^{40b}), OR⁴⁰, N(R^{40a})(R^{40b}), N(R⁴⁰)C(═O)R³⁹, N(R⁴⁰)C(═O)OR⁴⁰, N(R⁴⁰)C(═O)N(R^{40a})(R^{40b});
wherein R³³ is selected from the group consisting of
(i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴¹;
(ii) C(═O)R⁴², C(═O)OR⁴³, C(═O)N(R^{43a})(R^{43b}), OR⁴³, N(R^{43a})(R^{43b}), N(R⁴³)C(═O)R⁴², N(R⁴³)C(═O)OR⁴³, N(R⁴³)C(═O)N(R^{43a})(R^{43b});
wherein R³⁷ is selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein R³⁸ is selected from the group consisting of
(i) halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁴;
(ii) C(═O)R⁴⁵, C(═O)OR⁴⁶, C(═O)N(R^{46a})(R^{46b}), OR⁴⁶, N(R^{46a})(R^{46b}), N(R⁴⁶)C(═O)R⁴⁵, N(R⁴⁶)C(═O)OR⁴⁶, N(R⁴⁶)C(═O)N(R^{46a})(R^{46b});
wherein R³⁹, R⁴⁰, R^{40a}, and R^{40b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁷;
wherein R⁴¹ is selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein R⁴², R⁴³, R^{43a}, and R^{43b} are independently selected from the group consisting of H, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴⁸;
wherein R⁴⁴ is selected from the group consisting of halogen, CN, NO₂, C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁴;
wherein R⁴⁵, R⁴⁶, R^{46a}, and R^{46b} are independently selected from the group consisting of H, C₁-C₄-alkyl, C₁-C₄-haloalkyl, C₂-C₄-alkenyl, C₂-C₄-haloalkenyl, C₂-C₄-alkynyl, and C₂-C₄-haloalkynyl;
wherein R⁴⁷ is selected from the group consisting of halogen, CN, NO₂, OH, O(C₁-C₄-alkyl), NH₂, NH(C₁-C₄-alkyl), and N(C₁-C₄-alkyl)(C₁-C₄-alkyl), C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R¹¹;
wherein R⁴⁸ is selected from the group consisting of halogen, CN, NO₂, OH, O(C₁-C₄-alkyl), NH₂, NH(C₁-C₄-alkyl), and N(C₁-C₄-alkyl)(C₁-C₄-alkyl), C₁-C₆-alkyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned moieties is independently unsubstituted or substituted with one or more, same or different substituents R⁵¹;
wherein R⁴⁹, R⁵⁰, and R⁵¹ are independently selected from the group consisting of halogen, CN, NO₂, C₁-C₆-alkyl, C₁-C₆-haloalkyl, OH, O(C₁-C₄-alkyl), NH₂, NH(C₁-C₄-alkyl), and N(C₁-C₄-alkyl)(C₁-C₄-alkyl);
wherein R⁵ is selected from the group consisting of a 5- to 6-membered partially unsaturated or fully unsaturated carbocyclic or heterocyclic ring and a 9- to 10-membered partially unsaturated or fully unsaturated carbobicyclic or heterobicyclic ring, wherein said heterocyclic ring comprises one or more, same or different heteroatoms selected from O, N or S, wherein said N- and/or S-atoms are independently oxidized or non-oxidized, and wherein each substitutable carbon or heteroatom in the aforementioned cyclic or bicyclic moieties is independently unsubstituted or substituted with one or more, same or different substituents R¹⁷;
wherein R¹⁷ is selected from the group consisting of halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, a 5- to 6-membered saturated, partially unsaturated or fully unsaturated carbocyclic or heterocyclic ring, wherein said heterocyclic ring comprises one or more, same or different heteroatoms selected from O, N or S, wherein said N- and/or S-atoms are independently oxidized or non-oxidized, C(═O)R¹⁹, C(═O)OR², C(═O)N(R^{20a})(R^{20b}), OR²⁰, N(R^{20a})(R^{20b}), N(R²⁰)C(═O)R¹⁹, N(R²⁰)C(═O)OR²⁰, and N(R²⁰)C(═O)N(R^{20a})(R^{20b});
and/or two R¹⁷ present on one C-atom together form ═O;
wherein R¹⁹, R²⁰, R^{20a}, and R^{20b} are independently selected from the group consisting of H, C₁-C₂-alkyl, and C₁-C₂-haloalkyl;
and wherein all other substituents have the meaning as defined in claim 1.

12. The compound according to claim 1, wherein said compound is selected from the group consisting 4-{8-amino-6-phenylimidazo[1,2-a]pyrazin-5-yl}-2-chlorophenol; 4-[8-amino-6-(furan-2-yl)imidazo[1,2-a]pyrazin-5-yl]-2-chlorophenol; 5-(2,6-dimethylpyridin-4-yl)-6-phenylimidazo[1,2-a]pyrazin-8-amine; 5-[2-methyl-6-(trifluoromethyl)pyridin-4-yl]-6-phenylimidazo[1,2-a]pyrazin-8-amine; 4-[8-amino-6-(4-fluorophenyl)imidazo[1,2-a]pyrazin-5-yl]-2-chlorophenol; 6-(4-fluorophenyl)-5-[2-methyl-6-(trifluoromethyl)pyridin-4-yl]imidazo[1,2-a]pyrazin-8-amine; 5-(2,6-dimethylpyridin-4-yl)-6-(4-fluorophenyl)imidazo[1,2-a]pyrazin-8-amine; 6-(3-fluorophenyl)-5-[2-methyl-6-(trifluoromethyl)pyridin-4-yl]imidazo[1,2-a]pyrazin-8-amine; 3-{8-amino-5-[2-methyl-6-(trifluoromethyl)pyridin-4-yl]imidazo[1,2-a]pyrazin-6-yl}benzonitrile; 3-[8-amino-5-(3-chloro-4-hydroxyphenyl)imidazo[1,2-a]pyrazin-6-yl]benzonitrile; 4-[8-amino-6-(3-fluorophenyl)imidazo[1,2-a]pyrazin-5-yl]-2-chlorophenol; 4-[8-amino-2-(3-nitrophenyl)-6-phenylimidazo[1,2-a]pyrazin-5-yl]-2-chlorophenol; 5-(1H-indazol-5-yl)-6-phenylimidazo[1,2-a]pyrazin-8-amine; 4-{8-amino-6-phenylimidazo[1,2-a]pyrazin-5-yl}-2,6-dichlorophenol; 4-{8-amino-2-cyclohexyl-6-phenylimidazo[1,2-a]pyrazin-5-yl}-2-chlorophenol; 4-{8-amino-6-phenylimidazo[1,2-a]pyrazin-5-yl}-2-bromo-6-chlorophenol; 4-{8-amino-6-[4-(trifluoromethyl)phenyl]imidazo[1,2-a]pyrazin-5-yl}-2-chlorophenol; 3-[8-amino-5-(3-chloro-4-hydroxyphenyl)-6-phenylimidazo[1,2-a]pyrazin-2-yl]benzonitrile; 4-[8-amino-5-(3-chloro-4-hydroxyphenyl)imidazo[1,2-a]pyrazin-6-yl]benzonitrile; 4-{8-amino-6-phenylimidazo[1,2-a]pyrazin-5-yl}-N-methylpyridin-2-amine; 4-{8-amino-2,6-diphenylimidazo[1,2-a]pyrazin-5-yl}-2-chlorophenol; 4-[8-amino-6-(3-fluorophenyl)imidazo[1,2-a]pyrazin-5-yl]phenol; 4-[8-amino-6-(3-fluorophenyl)imidazo[1,2-a]pyrazin-5-yl]-N-methylpyridin-2-amine; 8-amino-5-(3-chloro-4-hydroxyphenyl)-6-phenylimidazo[1,2-a]pyrazine-2-carboxamide; 4-[8-amino-6-(4-fluorophenyl)imidazo[1,2-a]pyrazin-5-yl]-N-methylpyridin-2-amine; 6-(3-fluorophenyl)-5-(quinolin-6-yl)imidazo[1,2-a]pyrazin-8-amine; 5-(3,5-dichlorophenyl)-6-(4-fluorophenyl)imidazo[1,2-a]pyrazin-8-amine; 6-(2-fluorophenyl)-5-[2-methyl-6-(trifluoromethyl)pyridin-4-yl]imidazo[1,2-a]pyrazin-8-amine; 3-[8-amino-5-(3,5-dichlorophenyl)imidazo[1,2-a]pyrazin-6-yl]benzonitrile; 5-(3,5-dichlorophenyl)-6-phenylimidazo[1,2-a]pyrazin-8-amine; 4-{8-amino-6-[3-(trifluoromethyl)phenyl]imidazo[1,2-a]pyrazin-5-yl}-2-chlorophenol; 5-(3-chloro-1H-indazol-5-yl)-6-phenylimidazo[1,2-a]pyrazin-8-amine; 5-(2-chloro-6-methylpyridin-4-yl)-6-(3-fluorophenyl)imidazo[1,2-a]pyrazin-8-amine; 5-(2-chloro-6-methylpyridin-4-yl)-6-(4-fluorophenyl)imidazo[1,2-a]pyrazin-8-amine; 4-[8-amino-6-(3-fluorophenyl)imidazo[1,2-a]pyrazin-5-yl]benzamide; 5-(3-methyl-1H-indazol-5-yl)-6-phenylimidazo[1,2-a]pyrazin-8-amine; 5-(1H-indol-5-yl)-6-phenylimidazo[1,2-a]pyrazin-8-amine; 6-(3-fluorophenyl)-5-(pyridin-4-yl)imidazo[1,2-a]pyrazin-8-amine; 6-(1-methyl-1H-pyrazol-3-yl)-5-[2-methyl-6-(trifluoromethyl)pyridin-4-yl]imidazo[1,2-a]pyrazin-8-amine; 5-(3-fluoro-1H-indazol-5-yl)-6-phenylimidazo[1,2-a]pyrazin-8-amine; 4-[8-amino-6-(1-methyl-1H-pyrazol-3-yl)imidazo[1,2-a]pyrazin-5-yl]-2-chlorophenol; 5-(1-benzofuran-5-yl)-6-(3-fluorophenyl)imidazo[1,2-a]pyrazin-8-amine; 4-[8-amino-6-(2-fluorophenyl)imidazo[1,2-a]pyrazin-5-yl]-2-chlorophenol; 6-(3-fluorophenyl)-5-(2-methoxypyridin-4-yl)imidazo[1,2-a]pyrazin-8-amine; 5-(2-fluoro-6-methylpyridin-4-yl)-6-(3-fluorophenyl)imidazo[1,2-a]pyrazin-8-amine; 4-{8-amino-2-methyl-6-phenylimidazo[1,2-a]pyrazin-5-yl}-2-chlorophenol; 4-{8-amino-6-phenylimidazo[1,2-a]pyrazin-5-yl}-6-fluoro-N-methylpyridin-2-amine; 3-{8-amino-5-[2-(methylamino)pyridin-4-yl]imidazo[1,2-a]pyrazin-6-yl}benzonitrile; 5-[2-methyl-6-(trifluoromethyl)pyridin-4-yl]-6-(naphthalen-2-yl)imidazo[1,2-a]pyrazin-8-amine; 4-[8-amino-5-(3-chloro-4-hydroxyphenyl)-6-phenylimidazo[1,2-a]pyrazin-2-yl]benzonitrile; 5-(2,6-dimethylpyridin-4-yl)-6-(3-fluorophenyl)imidazo[1,2-a]pyrazin-8-amine; 4-[8-amino-6-(3-fluorophenyl)imidazo[1,2-a]pyrazin-5-yl]-2-chloro-6-methylphenol; 4-[8-amino-6-(3,5-difluorophenyl)imidazo[1,2-a]pyrazin-5-yl]-2-chlorophenol; 4-[8-amino-6-(3-fluorophenyl)imidazo[1,2-a]pyrazin-5-yl]-2,6-dichlorophenol; 5-(1,3-benzothiazol-5-yl)-6-(3-fluorophenyl)imidazo[1,2-a]pyrazin-8-amine; 4-[8-amino-6-(3-fluorophenyl)imidazo[1,2-a]pyrazin-5-yl]-2,6-dimethoxyphenol; 4-[8-amino-6-(4-fluorophenyl)imidazo[1,2-a]pyrazin-5-yl]-N,N,6-trimethylpyridin-2-amine; 4-[8-amino-6-(4-fluorophenyl)imidazo[1,2-a]pyrazin-5-yl]-N,6-dimethylpyridin-2-amine; 6-(3-fluorophenyl)-5-(1-methyl-1H-indazol-6-yl)imidazo[1,2-a]pyrazin-8-amine; 6-(3-fluorophenyl)-5-(8-fluoroquinolin-6-yl)imidazo[1,2-a]pyrazin-8-amine; 5-(1,3-benzothiazol-6-yl)-6-(3-fluorophenyl)imidazo[1,2-a]pyrazin-8-amine; 5,6-bis(1,3-benzothiazol-6-yl)imidazo[1,2-a]pyrazin-8-amine; 6-(3-fluorophenyl)-5-(7-methyl-1H-indazol-5-yl)imidazo[1,2-a]pyrazin-8-amine; 4-[8-amino-6-(3-fluoro-5-methoxyphenyl)imidazo[1,2-a]pyrazin-5-yl]-2-chlorophenol; 4-[8-amino-6-(3-fluorophenyl)imidazo[1,2-a]pyrazin-5-yl]-2,6-difluorophenol; ethyl 8-amino-6-(3-fluorophenyl)-5-[2-methyl-6-(trifluoromethyl)pyridin-4-yl]imidazo[1,2-a]pyrazine-2-carboxylate; 4-[8-amino-6-(3-fluorophenyl)imidazo[1,2-a]pyrazin-5-yl]-2-chloro-6-methoxyphenol; 8-amino-6-(3-fluorophenyl)-5-[2-methyl-6-(trifluoromethyl)pyridin-4-yl]imidazo[1,2-a]pyrazine-2-carboxamide; 6-(3-fluorophenyl)-5-(2-methylpyridin-4-yl)imidazo[1,2-a]pyrazin-8-amine; 8-amino-5-(3-chloro-4-hydroxyphenyl)-6-(3-fluorophenyl)imidazo[1,2-a]pyrazine-2-carboxylic acid; 5-(2,6-dichloropyridin-4-yl)-6-(3-fluorophenyl)imidazo[1,2-a]pyrazin-8-amine; 4-[8-amino-6-(3-fluorophenyl)imidazo[1,2-a]pyrazin-5-yl]-N,6-dimethylpyridin-2-amine; 6-(3-fluorophenyl)-5-{imidazo[1,2-a]pyridin-6-yl}imidazo[1,2-a]pyrazin-8-amine; 4-[8-amino-6-(3-fluorophenyl)imidazo[1,2-a]pyrazin-5-yl]-2,6-dimethylphenol; 8-amino-6-(3-fluorophenyl)-5-[2-methyl-6-(trifluoromethyl)pyridin-4-yl]imidazo[1,2-a]pyrazine-2-carboxylic acid; 8-amino-6-(3-fluorophenyl)-N,N-dimethyl-5-[2-methyl-6-(trifluoromethyl)pyridin-4-yl]imidazo[1,2-a]pyrazine-2-carboxamide; 8-amino-6-(3-fluorophenyl)-N-methyl-5-[2-methyl-6-(trifluoromethyl)pyridin-4-yl]imidazo[1,2-a]pyrazine-2-carboxamide; 5-(4-amino-3,5-dichlorophenyl)-6-(3-fluorophenyl)imidazo[1,2-a]pyrazin-8-amine; 6-(3-fluorophenyl)-5-(isoquinolin-6-yl)imidazo[1,2-a]pyrazin-8-amine; 6-(3-fluorophenyl)-5-(2-methoxy-6-methylpyridin-4-yl)imidazo[1,2-a]pyrazin-8-amine; 5-(1H-1,3-benzodiazol-6-yl)-6-(3-fluorophenyl)imidazo[1,2-a]pyrazin-8-amine; 6-(3-fluorophenyl)-5-(1-methyl-H-1,3-benzodiazol-6-yl)imidazo[1,2-a]pyrazin-8-amine; ethyl 8-amino-5-(3-chloro-4-hydroxyphenyl)-6-(3-fluorophenyl)imidazo[1,2-a]pyrazine-2-carboxylate; 4-[8-amino-6-(4-fluorophenyl)imidazo[1,2-a]pyrazin-5-yl]-6-methyl-N-(propan-2-yl)pyridin-2-amine; 6-(3-fluorophenyl)-5-(4-methyl-1,3-benzothiazol-6-yl)imidazo[1,2-a]pyrazin-8-amine; 8-amino-6-(3-fluorophenyl)-5-[2-methyl-6-(trifluoromethyl)pyridin-4-yl]-N-(oxolan-3-yl)imidazo[1,2-a]pyrazine-2-carboxamide; 5-(8-chloroquinolin-6-yl)-6-(3-fluorophenyl)imidazo[1,2-a]pyrazin-8-amine; 4-[8-amino-6-(3-fluorophenyl)-2-(4-methylpiperazine-1-carbonyl)imidazo[1,2-a]pyrazin-5-yl]-2-chlorophenol; 6-[8-amino-6-(3-fluorophenyl)imidazo[1,2-a]pyrazin-5-yl]-1-methyl-2,3-dihydro-1H-1,3-benzodiazol-2-one; 5-[8-amino-6-(3-fluorophenyl)imidazo[1,2-a]pyrazin-5-yl]-2,3-dihydro-1H-indol-2-one; 6-(3-fluorophenyl)-5-(quinoxalin-6-yl)imidazo[1,2-a]pyrazin-8-amine; 5-(2-chloropyridin-4-yl)-6-(3-fluorophenyl)imidazo[1,2-a]pyrazin-8-amine; 5-(4-fluoro-1,3-benzothiazol-6-yl)-6-(3-fluorophenyl)imidazo[1,2-a]pyrazin-8-amine; 5-[8-amino-6-(3-fluorophenyl)imidazo[1,2-a]pyrazin-5-yl]-3-bromopyridin-2-ol; 5-[8-amino-6-(3-fluorophenyl)imidazo[1,2-a]pyrazin-5-yl]-1,2-dihydropyridin-2-one; 8-amino-5-(3-chloro-4-hydroxyphenyl)-6-(3-fluorophenyl)imidazo[1,2-a]pyrazine-2-carboxamide; 6-(3-fluorophenyl)-5-[2-methyl-6-(trifluoromethyl)pyridin-4-yl]-2-phenylimidazo[1,2-a]pyrazin-8-amine; 5-[8-amino-6-(3-fluorophenyl)imidazo[1,2-a]pyrazin-5-yl]-1,3-dimethyl-1,2-dihydropyridin-2-one; 6-(3-fluorophenyl)-5-[2-(pyrrolidin-1-yl)pyridin-4-yl]imidazo[1,2-a]pyrazin-8-amine; 4-[8-amino-2-(aminomethyl)-6-phenylimidazo[1,2-a]pyrazin-5-yl]-2-chlorophenol; 6-(3-fluorophenyl)-5-{pyrazolo[1,5-a]pyrimidin-6-yl}imidazo[1,2-a]pyrazin-8-amine; 6-(3-fluorophenyl)-5-(8-methylquinolin-6-yl)imidazo[1,2-a]pyrazin-8-amine; 6-(4-fluorophenyl)-5-(8-fluoroquinolin-6-yl)imidazo[1,2-a]pyrazin-8-amine; 6-(4-fluorophenyl)-5-(1-methyl-1H-1,2,3-benzotriazol-6-yl)imidazo[1,2-a]pyrazin-8-amine; 6-[8-amino-6-(3-fluorophenyl)imidazo[1,2-a]pyrazin-5-yl]-1,3-benzothiazol-2-amine; 6-(3-fluorophenyl)-5-(8-fluoroquinoxalin-6-yl)imidazo[1,2-a]pyrazin-8-amine; 6-(3-fluorophenyl)-5-{8-methylimidazo[1,2-a]pyridin-6-yl}imidazo[1,2-a]pyrazin-8-amine; 3-[8-amino-5-(8-fluoroquinolin-6-yl)imidazo[1,2-a]pyrazin-6-yl]benzonitrile; N-{4-[8-amino-6-(3-fluorophenyl)imidazo[1,2-a]pyrazin-5-yl]-6-methylpyridin-2-yl}acetamide; 6-(3-fluorophenyl)-5-[8-(trifluoromethyl)quinolin-6-yl]imidazo[1,2-a]pyrazin-8-amine; 6-(3-fluorophenyl)-5-(8-methoxyquinolin-6-yl)imidazo[1,2-a]pyrazin-8-amine; 6-(3-fluorophenyl)-5-(1,8-naphthyridin-3-yl)imidazo[1,2-a]pyrazin-8-amine; 6-(3-fluorophenyl)-5-(7-fluoroquinolin-6-yl)imidazo[1,2-a]pyrazin-8-amine; 5-(4-fluoro-1-methyl-1H-1,3-benzodiazol-6-yl)-6-(3-fluorophenyl)imidazo[1,2-a]pyrazin-8-amine; 6-(3-fluorophenyl)-5-(1,8-naphthyridin-4-yl)imidazo[1,2-a]pyrazin-8-amine; ethyl 8-amino-6-(3-fluorophenyl)-5-(8-fluoroquinolin-6-yl)imidazo[1,2-a]pyrazine-2-carboxylate; [8-amino-6-(3-fluorophenyl)-5-(8-fluoroquinolin-6-yl)imidazo[1,2-a]pyrazin-2-yl]methanol; 6-(3-fluorophenyl)-5-[2-methyl-6-(pyrrolidin-1-yl)pyridin-4-yl]imidazo[1,2-a]pyrazin-8-amine; 5-{8-fluoroimidazo[1,2-a]pyridin-6-yl}-6-(3-fluorophenyl)imidazo[1,2-a]pyrazin-8-amine; 2-[8-amino-5-(8-fluoroquinolin-6-yl)imidazo[1,2-a]pyrazin-6-yl]phenol; 6-(6-fluoropyridin-2-yl)-5-(8-fluoroquinolin-6-yl)imidazo[1,2-a]pyrazin-8-amine; 5-[8-amino-6-(3-fluorophenyl)imidazo[1,2-a]pyrazin-5-yl]-1-ethyl-1,2-dihydropyridin-2-one; 6-(3-fluorophenyl)-5-{1H-pyrrolo[2,3-b]pyridin-3-yl}imidazo[1,2-a]pyrazin-8-amine; 5-(5,8-difluoroquinolin-6-yl)-6-(3-fluorophenyl)imidazo[1,2-a]pyrazin-8-amine; 6-[8-amino-6-(3-fluorophenyl)imidazo[1,2-a]pyrazin-5-yl]quinolin-8-amine; ethyl 2-[8-amino-6-(3-fluorophenyl)-5-(8-fluoroquinolin-6-yl)imidazo[1,2-a]pyrazin-2-yl]acetate; 5-(7-fluoro-1H-indazol-5-yl)-6-(3-fluorophenyl)imidazo[1,2-a]pyrazin-8-amine; 6-(3-fluorophenyl)-5-(4-methylquinolin-6-yl)imidazo[1,2-a]pyrazin-8-amine; 6-[8-amino-6-(3-fluorophenyl)imidazo[1,2-a]pyrazin-5-yl]quinoline-8-carbonitrile; 5-{8-fluoro-[1,2,4]triazolo[1,5-a]pyridin-6-yl}-6-(3-fluorophenyl)imidazo[1,2-a]pyrazin-8-amine; 5-(4-fluoro-1H-1,3-benzodiazol-6-yl)-6-(3-fluorophenyl)imidazo[1,2-a]pyrazin-8-amine; 4-[8-amino-6-(3-fluorophenyl)imidazo[1,2-a]pyrazin-5-yl]-2-fluoro-6-(trifluoromethyl)phenol; 6-[8-amino-6-(3-fluorophenyl)imidazo[1,2-a]pyrazin-5-yl]isoquinolin-1-ol; 2-[8-amino-6-(3-fluorophenyl)-5-(8-fluoroquinolin-6-yl)imidazo[1,2-a]pyrazin-2-yl]acetic acid; 5-[8-amino-6-(3-fluorophenyl)imidazo[1,2-a]pyrazin-5-yl]-2,3-dihydro-1H-isoindol-1-one; 5-[8-amino-6-(3-fluorophenyl)imidazo[1,2-a]pyrazin-5-yl]-2,3-dihydro-1H-inden-1-one; 2-[8-amino-6-(3-fluorophenyl)-5-(8-fluoroquinolin-6-yl)imidazo[1,2-a]pyrazin-2-yl]ethan-1-ol; 2-[8-amino-6-(3-fluorophenyl)-5-(8-fluoroquinolin-6-yl)imidazo[1,2-a]pyrazin-2-yl]acetamide; 6-(3-fluorophenyl)-5-(4-methoxy-1,3-benzothiazol-6-yl)imidazo[1,2-a]pyrazin-8-amine; 6-[8-amino-6-(3-fluorophenyl)imidazo[1,2-a]pyrazin-5-yl]naphthalen-1-ol; 5-[4-fluoro-1-(propan-2-yl)-1H-1,3-benzodiazol-6-yl]-6-(3-fluorophenyl)imidazo[1,2-a]pyrazin-8-amine; 6-(3-fluorophenyl)-5-(3-methyl-1H-indazol-5-yl)imidazo[1,2-a]pyrazin-8-amine; 5-[8-amino-6-(3-fluorophenyl)imidazo[1,2-a]pyrazin-5-yl]-1-ethyl-3-fluoro-1,2-dihydropyridin-2-one; 6-[8-amino-6-(3-fluorophenyl)imidazo[1,2-a]pyrazin-5-yl]quinolin-3-amine; 5-(4-fluoro-1,3-benzothiazol-6-yl)-6-(4-fluorophenyl)imidazo[1,2-a]pyrazin-8-amine; 3-[8-amino-5-(4-fluoro-1,3-benzothiazol-6-yl)imidazo[1,2-a]pyrazin-6-yl]benzonitrile; 6-(4-fluorophenyl)-5-(4-methylquinolin-6-yl)imidazo[1,2-a]pyrazin-8-amine; 5-(1,3-benzothiazol-6-yl)-6-(4-fluorophenyl)imidazo[1,2-a]pyrazin-8-amine; 6-(3-fluorophenyl)-5-(quinazolin-6-yl)imidazo[1,2-a]pyrazin-8-amine; 5-(8-chloroquinolin-6-yl)-6-(4-fluorophenyl)imidazo[1,2-a]pyrazin-8-amine; 5-(8-fluoro-4-methylquinolin-6-yl)-6-(4-fluorophenyl)imidazo[1,2-a]pyrazin-8-amine; 6-(4-fluorophenyl)-5-(1-methyl-H-1,3-benzodiazol-6-yl)imidazo[1,2-a]pyrazin-8-amine; 5-(4-fluoro-1-methyl-1H-1,3-benzodiazol-6-yl)-6-(4-fluorophenyl)imidazo[1,2-a]pyrazin-8-amine; 6-(3-fluorophenyl)-5-{3-methylimidazo[1,2-a]pyridin-6-yl}imidazo[1,2-a]pyrazin-8-amine; 3-(8-amino-5-{8-methylimidazo[1,2-a]pyridin-6-yl}imidazo[1,2-a]pyrazin-6-yl)benzonitrile; 3-[8-amino-5-(8-chloroquinolin-6-yl)imidazo[1,2-a]pyrazin-6-yl]benzonitrile; 3-[8-amino-5-(1,3-benzothiazol-6-yl)imidazo[1,2-a]pyrazin-6-yl]benzonitrile; 6-(3-fluorophenyl)-5-[5-(1H-pyrazol-5-yl)thiophen-2-yl]imidazo[1,2-a]pyrazin-8-amine; 3-[8-amino-5-(4-methylquinolin-6-yl)imidazo[1,2-a]pyrazin-6-yl]benzonitrile; 3-[8-amino-5-(8-methoxyquinolin-6-yl)imidazo[1,2-a]pyrazin-6-yl]benzonitrile; 3-[8-amino-5-(1-methyl-1H-1,3-benzodiazol-6-yl)imidazo[1,2-a]pyrazin-6-yl]benzonitrile; 6-[8-amino-6-(3-fluorophenyl)imidazo[1,2-a]pyrazin-5-yl]-N-methylquinolin-8-amine; 6-[8-amino-6-(3-fluorophenyl)imidazo[1,2-a]pyrazin-5-yl]-N,N-dimethylquinolin-8-amine; 5-(4-chloro-1,3-benzothiazol-6-yl)-6-(4-fluorophenyl)imidazo[1,2-a]pyrazin-8-amine; 8-amino-6-(3-cyanophenyl)-5-(quinolin-6-yl)imidazo[1,2-a]pyrazine-2-carboxamide; 2-[8-amino-6-(3-cyanophenyl)-5-(quinolin-6-yl)imidazo[1,2-a]pyrazin-2-yl]acetamide; 6-(4-fluorophenyl)-5-(2-methylquinolin-6-yl)imidazo[1,2-a]pyrazin-8-amine; 3-[8-amino-5-(8-aminoquinolin-6-yl)imidazo[1,2-a]pyrazin-6-yl]benzonitrile; 6-(4-fluorophenyl)-5-(3-fluoroquinolin-6-yl)imidazo[1,2-a]pyrazin-8-amine; 5-(3,5-dichloro-4-methoxyphenyl)-6-phenylimidazo[1,2-a]pyrazin-8-amine; 6-(4-fluorophenyl)-5-(2-fluoroquinolin-6-yl)imidazo[1,2-a]pyrazin-8-amine; methyl 5-[8-amino-6-(4-fluorophenyl)imidazo[1,2-a]pyrazin-5-yl]furan-2-carboxylate; 5-[8-amino-6-(4-fluorophenyl)imidazo[1,2-a]pyrazin-5-yl]-1,2-dihydropyridin-2-one; 3-[8-amino-5-(3-aminoquinolin-6-yl)imidazo[1,2-a]pyrazin-6-yl]benzonitrile; 3-(8-amino-5-{3-methylimidazo[1,2-a]pyridin-6-yl}imidazo[1,2-a]pyrazin-6-yl)benzonitrile; 3-[8-amino-5-(5-fluoroquinolin-6-yl)imidazo[1,2-a]pyrazin-6-yl]benzonitrile; 6-[8-amino-6-(4-fluorophenyl)imidazo[1,2-a]pyrazin-5-yl]imidazo[1,2-a]pyridine-3-carbonitrile; 5-[8-amino-6-(4-fluorophenyl)imidazo[1,2-a]pyrazin-5-yl]-1-methyl-1,2-dihydropyridin-2-one; 5-[8-amino-6-(3-fluorophenyl)imidazo[1,2-a]pyrazin-5-yl]-1-methyl-,2-dihydropyridin-2-one; 6-[8-amino-6-(3-fluorophenyl)imidazo[1,2-a]pyrazin-5-yl]imidazo[1,2-a]pyridine-3-carbonitrile; 5-(4,8-dimethylquinolin-6-yl)-6-(4-fluorophenyl)imidazo[1,2-a]pyrazin-8-amine; 5-(1H-1,3-benzodiazol-6-yl)-6-(4-fluorophenyl)imidazo[1,2-a]pyrazin-8-amine; 6-(4-fluorophenyl)-5-{3-methylimidazo[1,2-a]pyridin-6-yl}imidazo[1,2-a]pyrazin-8-amine; 6-(4-fluorophenyl)-5-(4-methoxy-1H-1,3-benzodiazol-6-yl)imidazo[1,2-a]pyrazin-8-amine; 6-(4-fluorophenyl)-5-(3-methylquinolin-6-yl)imidazo[1,2-a]pyrazin-8-amine; 5-[8-amino-6-(4-fluorophenyl)imidazo[1,2-a]pyrazin-5-yl]-1-(difluoromethyl)-1,2-dihydropyridin-2-one; 1-{4-[8-amino-6-(4-fluorophenyl)imidazo[1,2-a]pyrazin-5-yl]pyridin-2-yl}ethan-1-one; 5-{8-fluoro-3-methylimidazo[1,2-a]pyridin-6-yl}-6-(3-fluorophenyl)imidazo[1,2-a]pyrazin-8-amine; 6-(4-fluorophenyl)-5-(4-methylquinazolin-6-yl)imidazo[1,2-a]pyrazin-8-amine; 4-{8-amino-2-cyclopropyl-6-phenylimidazo[1,2-a]pyrazin-5-yl}-2-chlorophenol; 6-[8-amino-6-(4-fluorophenyl)imidazo[1,2-a]pyrazin-5-yl]quinolin-3-amine; 6-(4-fluorophenyl)-5-{2-methylimidazo[1,2-a]pyridin-6-yl}imidazo[1,2-a]pyrazin-8-amine; 6-(4-fluorophenyl)-5-{imidazo[1,2-a]pyridin-6-yl}imidazo[1,2-a]pyrazin-8-amine; 6-(4-fluorophenyl)-5-(3-fluoropyridin-4-yl)imidazo[1,2-a]pyrazin-8-amine; 6-(3-fluorophenyl)-5-(3-fluoroquinolin-6-yl)imidazo[1,2-a]pyrazin-8-amine; 3-(8-amino-5-{imidazo[1,2-a]pyridin-6-yl}imidazo[1,2-a]pyrazin-6-yl)benzonitrile; 5-[8-amino-6-(4-fluorophenyl)imidazo[1,2-a]pyrazin-5-yl]-3-methyl-1,2-dihydropyridin-2-one; 3-[8-amino-5-(1H-1,3-benzodiazol-6-yl)imidazo[1,2-a]pyrazin-6-yl]benzonitrile; 6-(4-fluorophenyl)-5-{[1,2,4]triazolo[4,3-a]pyridin-6-yl}imidazo[1,2-a]pyrazin-8-amine; 5-{3-ethylimidazo[1,2-a]pyridin-6-yl}-6-(4-fluorophenyl)imidazo[1,2-a]pyrazin-8-amine; 6-(4-fluorophenyl)-5-{pyrazolo[1,5-a]pyridin-5-yl}imidazo[1,2-a]pyrazin-8-amine; 5-[8-amino-6-(4-fluorophenyl)imidazo[1,2-a]pyrazin-5-yl]-3-fluoro-1,2-dihydropyridin-2-one; 4-{8-amino-5-[2-methyl-6-(trifluoromethyl)pyridin-4-yl]imidazo[1,2-a]pyrazin-6-yl}benzonitrile; 4-[8-amino-5-(8-fluoroquinolin-6-yl)imidazo[1,2-a]pyrazin-6-yl]benzonitrile; 6-(3-fluorophenyl)-5-{1H,2H,3H-pyrrolo[2,3-b]pyridin-4-yl}imidazo[1,2-a]pyrazin-8-amine; 5-(8-fluoroquinolin-6-yl)-6-(pyridin-3-yl)imidazo[1,2-a]pyrazin-8-amine; 6-(2-fluoropyridin-4-yl)-5-(4-methylquinolin-6-yl)imidazo[1,2-a]pyrazin-8-amine; 5-[8-amino-5-(1-methyl-1H-1,3-benzodiazol-6-yl)imidazo[1,2-a]pyrazin-6-yl]-2-fluorobenzonitrile; 5-[8-amino-6-(5-methylfuran-2-yl)imidazo[1,2-a]pyrazin-5-yl]-1,2-dihydropyridin-2-one; 5-[8-amino-6-(1-methyl-1H-pyrazol-3-yl)imidazo[1,2-a]pyrazin-5-yl]-1,2-dihydropyridin-2-one; 5-[8-amino-5-(4-methylquinolin-6-yl)imidazo[1,2-a]pyrazin-6-yl]-2-fluorobenzonitrile; 6-(3-methoxyphenyl)-5-(4-methylquinolin-6-yl)imidazo[1,2-a]pyrazin-8-amine; 6-(1-methyl-1H-pyrazol-3-yl)-5-(4-methylquinolin-6-yl)imidazo[1,2-a]pyrazin-8-amine; 4-[8-amino-5-(4-methylquinolin-6-yl)imidazo[1,2-a]pyrazin-6-yl]-2-fluorobenzonitrile; 6-(5-methylfuran-2-yl)-5-(4-methylquinolin-6-yl)imidazo[1,2-a]pyrazin-8-amine; {4-[8-amino-5-(quinolin-6-yl)imidazo[1,2-a]pyrazin-6-yl]thiophen-2-yl}methanol; 6-(6-fluoropyridin-2-yl)-5-(4-methylquinolin-6-yl)imidazo[1,2-a]pyrazin-8-amine; 1-{4-[8-amino-5-(quinolin-6-yl)imidazo[1,2-a]pyrazin-6-yl]pyridin-2-yl}ethan-1-one; 5-(4-methylquinolin-6-yl)-6-(pyridin-4-yl)imidazo[1,2-a]pyrazin-8-amine; 4-[8-amino-5-(4-methylquinolin-6-yl)imidazo[1,2-a]pyrazin-6-yl]benzonitrile; 4-[8-amino-5-(8-chloroquinolin-6-yl)imidazo[1,2-a]pyrazin-6-yl]benzonitrile; 4-[8-amino-5-(quinolin-6-yl)imidazo[1,2-a]pyrazin-6-yl]benzonitrile; {5-[8-amino-5-(quinolin-6-yl)imidazo[1,2-a]pyrazin-6-yl]furan-2-yl}methanol; 4-[8-amino-5-(quinolin-6-yl)imidazo[1,2-a]pyrazin-6-yl]pyridine-2-carbonitrile; 5-(quinolin-6-yl)-6-(1,3-thiazol-4-yl)imidazo[1,2-a]pyrazin-8-amine; 6-(3-aminophenyl)-5-(quinolin-6-yl)imidazo[1,2-a]pyrazin-8-amine; 2-{4-[8-amino-5-(quinolin-6-yl)imidazo[1,2-a]pyrazin-6-yl]-1H-pyrazol-1-yl}ethan-1-ol; 5-[8-amino-5-(quinolin-6-yl)imidazo[1,2-a]pyrazin-6-yl]pyridine-3-carbonitrile; 5-[8-amino-5-(quinolin-6-yl)imidazo[1,2-a]pyrazin-6-yl]thiophene-2-carbonitrile; 6-(2-methylpyridin-4-yl)-5-(quinolin-6-yl)imidazo[1,2-a]pyrazin-8-amine; 5-[8-amino-5-(quinolin-6-yl)imidazo[1,2-a]pyrazin-6-yl]pyridin-2-amine; 6-(2-methoxypyridin-4-yl)-5-(quinolin-6-yl)imidazo[1,2-a]pyrazin-8-amine; 6-(3-methoxyphenyl)-5-(quinolin-6-yl)imidazo[1,2-a]pyrazin-8-amine; 6-(3-nitrophenyl)-5-(quinolin-6-yl)imidazo[1,2-a]pyrazin-8-amine; 6-(6-methoxypyridin-3-yl)-5-(quinolin-6-yl)imidazo[1,2-a]pyrazin-8-amine; methyl 5-[8-amino-5-(quinolin-6-yl)imidazo[1,2-a]pyrazin-6-yl]furan-2-carboxylate; 5-[8-amino-5-(quinolin-6-yl)imidazo[1,2-a]pyrazin-6-yl]-3-methylpyridine-2-carbonitrile; 3-[8-amino-5-(quinolin-6-yl)imidazo[1,2-a]pyrazin-6-yl]phenol; 5-(8-fluoroquinolin-6-yl)-6-(furan-2-yl)imidazo[1,2-a]pyrazin-8-amine; 6-(4-methoxyphenyl)-5-(quinolin-6-yl)imidazo[1,2-a]pyrazin-8-amine; 6-(6-fluoropyridin-2-yl)-5-(quinolin-6-yl)imidazo[1,2-a]pyrazin-8-amine; 6-(pyridin-4-yl)-5-(quinolin-6-yl)imidazo[1,2-a]pyrazin-8-amine; 5-(8-fluoroquinolin-6-yl)-6-(6-methoxypyridin-3-yl)imidazo[1,2-a]pyrazin-8-amine; 6-(6-fluoropyridin-3-yl)-5-(8-fluoroquinolin-6-yl)imidazo[1,2-a]pyrazin-8-amine; 6-(3,4-difluorophenyl)-5-(8-fluoroquinolin-6-yl)imidazo[1,2-a]pyrazin-8-amine; 5-(8-fluoroquinolin-6-yl)-6-[4-(trifluoromethyl)phenyl]imidazo[1,2-a]pyrazin-8-amine; 6-(furan-2-yl)-5-(quinolin-6-yl)imidazo[1,2-a]pyrazin-8-amine; 6-(5-methylfuran-2-yl)-5-(quinolin-6-yl)imidazo[1,2-a]pyrazin-8-amine; 6-(pyridin-3-yl)-5-(quinolin-6-yl)imidazo[1,2-a]pyrazin-8-amine; 6-(1-methyl-1H-pyrazol-3-yl)-5-(quinolin-6-yl)imidazo[1,2-a]pyrazin-8-amine; {3-[8-amino-5-(quinolin-6-yl)imidazo[1,2-a]pyrazin-6-yl]phenyl}methanol; 6-(5-fluoropyridin-3-yl)-5-(quinolin-6-yl)imidazo[1,2-a]pyrazin-8-amine; 6-(6-fluoropyridin-3-yl)-5-(quinolin-6-yl)imidazo[1,2-a]pyrazin-8-amine; 6-(4-fluorophenyl)-5-(quinolin-6-yl)imidazo[1,2-a]pyrazin-8-amine; 5-(quinolin-6-yl)-6-[3-(trifluoromethyl)phenyl]imidazo[1,2-a]pyrazin-8-amine; 6-(3-aminophenyl)-5-(8-fluoroquinolin-6-yl)imidazo[1,2-a]pyrazin-8-amine; 3-[8-amino-5-(8-fluoroquinolin-6-yl)imidazo[1,2-a]pyrazin-6-yl]phenol; 6-(1,3-benzothiazol-6-yl)-5-(8-fluoroquinolin-6-yl)imidazo[1,2-a]pyrazin-8-amine; 5-(8-fluoroquinolin-6-yl)-6-(4-methoxyphenyl)imidazo[1,2-a]pyrazin-8-amine; 5-(8-fluoroquinolin-6-yl)-6-(1H-pyrazol-5-yl)imidazo[1,2-a]pyrazin-8-amine; 3-[8-amino-5-(quinolin-6-yl)imidazo[1,2-a]pyrazin-6-yl]benzonitrile; 5-[8-amino-6-(5-methylfuran-2-yl)imidazo[1,2-a]pyrazin-5-yl]-1-ethyl-1,2-dihydropyridin-2-one; 6-(5-chloro-6-methoxypyridin-3-yl)-5-(quinolin-6-yl)imidazo[1,2-a]pyrazin-8-amine; 1-{5-[8-amino-5-(quinolin-6-yl)imidazo[1,2-a]pyrazin-6-yl]pyridin-2-yl}ethan-1-one; 6-(3,4-difluorophenyl)-5-(4-methylquinolin-6-yl)imidazo[1,2-a]pyrazin-8-amine; 5-(4-methylquinolin-6-yl)-6-(1,3-thiazol-4-yl)imidazo[1,2-a]pyrazin-8-amine; 8-amino-6-(3-fluorophenyl)-5-(4-methylquinolin-6-yl)imidazo[1,2-a]pyrazine-2-carboxamide; 8-amino-6-(3-fluorophenyl)-N-methyl-5-{3-methylimidazo[1,2-a]pyridin-6-yl}imidazo[1,2-a]pyrazine-2-carboxamide; 8-amino-6-(4-fluorophenyl)-5-(4-methylquinolin-6-yl)imidazo[1,2-a]pyrazine-2-carboxamide; 8-amino-6-(3-fluorophenyl)-5-{3-methylimidazo[1,2-a]pyridin-6-yl}imidazo[1,2-a]pyrazine-2-carboxamide; 8-amino-6-(4-fluorophenyl)-N-methyl-5-(4-methylquinolin-6-yl)imidazo[1,2-a]pyrazine-2-carboxamide; 8-amino-6-(3-fluorophenyl)-5-(1-methyl-1H-1,3-benzodiazol-6-yl)imidazo[1,2-a]pyrazine-2-carboxamide; 8-amino-6-(4-fluorophenyl)-5-(quinolin-6-yl)imidazo[1,2-a]pyrazine-2-carboxamide; ethyl 8-amino-6-(4-fluorophenyl)-5-(4-methylquinolin-6-yl)imidazo[1,2-a]pyrazine-2-carboxylate; ethyl 8-amino-6-(3-cyanophenyl)-5-(8-fluoroquinolin-6-yl)imidazo[1,2-a]pyrazine-2-carboxylate; 6-(4-fluorophenyl)-5-(4-methylquinolin-6-yl)-2-(trifluoromethyl)imidazo[1,2-a]pyrazin-8-amine; 6-(4-fluorophenyl)-5-(quinolin-6-yl)-2-(trifluoromethyl)imidazo[1,2-a]pyrazin-8-amine; 8-amino-6-(4-fluorophenyl)-N-methyl-5-(1-methyl-H-1,3-benzodiazol-6-yl)imidazo[1,2-a]pyrazine-2-carboxamide; 6-(3-fluorophenyl)-5-(1-methyl-H-1,3-benzodiazol-6-yl)-2-(morpholine-4-carbonyl)imidazo[1,2-a]pyrazin-8-amine; 6-(3-fluorophenyl)-5-(8-fluoroquinolin-6-yl)-2-[4-(4-methoxybenzoyl)piperazine-1-carbonyl]imidazo[1,2-a]pyrazin-8-amine; 2-[4-(2,4-difluorophenyl)piperazine-1-carbonyl]-6-(3-fluorophenyl)-5-(1-methyl-1H-1,3-benzodiazol-6-yl)imidazo[1,2-a]pyrazin-8-amine; ethyl 8-amino-6-(4-fluorophenyl)-5-(quinolin-6-yl)imidazo[1,2-a]pyrazine-2-carboxylate; 1-[8-amino-6-(4-fluorophenyl)-5-(1-methyl-1H-1,3-benzodiazol-6-yl)imidazo[1,2-a]pyrazine-2-carbonyl]-4-methylpiperidin-4-ol; 8-amino-6-(3-fluorophenyl)-N,N-dimethyl-5-(1-methyl-1H-1,3-benzodiazol-6-yl)imidazo[1,2-a]pyrazine-2-carboxamide; 6-(4-fluorophenyl)-2-(4-methylpiperazine-1-carbonyl)-5-(4-methylquinolin-6-yl)imidazo[1,2-a]pyrazin-8-amine; 8-amino-6-(4-fluorophenyl)-5-{3-methylimidazo[1,2-a]pyridin-6-yl}imidazo[1,2-a]pyrazine-2-carboxamide; 8-amino-6-(4-fluorophenyl)-N-(2-methoxyethyl)-5-(1-methyl-1H-1,3-benzodiazol-6-yl)imidazo[1,2-a]pyrazine-2-carboxamide; 8-amino-6-(4-fluorophenyl)-N,N-dimethyl-5-(4-methylquinolin-6-yl)imidazo[1,2-a]pyrazine-2-carboxamide; 2-[4-(2,4-difluorophenyl)piperazine-1-carbonyl]-6-(4-fluorophenyl)-5-(1-methyl-1H-1,3-benzodiazol-6-yl)imidazo[1,2-a]pyrazin-8-amine; 8-amino-N-({1-[(2,4-difluorophenyl)methyl]piperidin-4-yl}methyl)-6-(4-fluorophenyl)-5-(1-methyl-1H-1,3-benzodiazol-6-yl)imidazo[1,2-a]pyrazine-2-carboxamide; 2-[8-amino-6-(4-fluorophenyl)-5-(1-methyl-1H-1,3-benzodiazol-6-yl)imidazo[1,2-a]pyrazin-2-yl]-1-[4-(2,4-difluorophenyl)piperazin-1-yl]ethan-1-one; 6-(4-fluorophenyl)-5-(4-methylquinolin-6-yl)-2-(piperazine-1-carbonyl)imidazo[1,2-a]pyrazin-8-amine; 6-(4-fluoro-3-methylphenyl)-5-(1-methyl-1H-1,3-benzodiazol-6-yl)imidazo[1,2-a]pyrazin-8-amine; 6-(6-fluoropyridin-3-yl)-5-(1-methyl-1H-1,3-benzodiazol-6-yl)imidazo[1,2-a]pyrazin-8-amine; 6-(4-fluorophenyl)-5-(1-methyl-1H-1,3-benzodiazol-6-yl)-2-(4-methylpiperazine-1-carbonyl)imidazo[1,2-a]pyrazin-8-amine; 6-(6-fluoropyridin-2-yl)-5-(1-methyl-1H-1,3-benzodiazol-6-yl)imidazo[1,2-a]pyrazin-8-amine; 2-[8-amino-6-(4-fluorophenyl)-5-(1-methyl-1H-1,3-benzodiazol-6-yl)imidazo[1,2-a]pyrazin-2-yl]acetamide; [8-amino-6-(4-fluorophenyl)-5-(1-methyl-1H-1,3-benzodiazol-6-yl)imidazo[1,2-a]pyrazin-2-yl]methanol; ethyl 8-amino-6-(4-fluorophenyl)-5-(1-methyl-1H-1,3-benzodiazol-6-yl)imidazo[1,2-a]pyrazine-2-carboxylate; 6-(4-fluorophenyl)-5-(4-methylquinolin-6-yl)-2-(morpholine-4-carbonyl)imidazo[1,2-a]pyrazin-8-amine; 5-(1-methyl-1H-1,3-benzodiazol-6-yl)-6-(pyridin-4-yl)imidazo[1,2-a]pyrazin-8-amine; 5-(1-ethyl-1H-1,3-benzodiazol-6-yl)-6-(4-fluorophenyl)imidazo[1,2-a]pyrazin-8-amine; and 1-[8-amino-6-(4-fluorophenyl)-5-(4-methylquinolin-6-yl)imidazo[1,2-a]pyrazine-2-carbonyl]-4-methylpiperidin-4-ol.

13. A pharmaceutical composition comprising a pharmaceutically effective amount of the compound according to claim 1, or a salt, stereoisomer, tautomer, isotopologue, or N-oxide thereof and a pharmaceutically acceptable carrier, diluent or excipient.

14. (canceled)

15. A method for treating a disease selected from the group consisting of cancer, Parkinson's disease, Huntington's disease, Alzheimer's disease, psychosis, stroke, extra pyramidal syndrome (in particular dystonia, akathisia, pseudoparkinsonism and tardive dyskinesia), attention deficit disorder (ADD), attention deficit hyperactivity disorder (ADHD), amyotrophic lateral sclerosis, cirrhosis, fibrosis, fatty liver, addictive behavior, dermal fibrosis (in particular dermal fibrosis in scleroderma), sleep disorders, AIDS, autoimmune diseases, infections, atherosclerosis and ischemia-reperfusion injury, comprising administering to a patient in need thereof a pharmaceutically effective amount of the compound as defined in claim 1, or a salt, stereoisomer, tautomer, isotopologue, or N-oxide thereof.

16. A method for treating cancer, comprising administering to a patient in need thereof a pharmaceutically effective amount of the compound as defined in claim 1, or a salt, stereoisomer, tautomer, isotopologue, or N-oxide thereof, wherein at least one further anti-neoplastic agent is coadministered with said compound.

17. The method according to claim 16, wherein said anti-neoplastic agent is selected from the group consisting of a chemotherapeutic agent, a topoisomerase II inhibitor, an antimetabolite, a topoisomerase I inhibitor, a hormone, a hormonal analogue, a signal transduction pathway inhibitor, a non-receptor tyrosine kinase inhibitor, an angiogenesis inhibitor, a proapoptotic agent, a cell cycle signaling inhibitor, a proteasome inhibitor, an inhibitors of cancer metabolism, and an immunotherapeutic agent.

18. The method according to claim 21, wherein said checkpoint inhibitor targets PD-1, PD-L1, CTLA-4, IDO, KIR, TIM-3, LAG-3, CD39, CD73, ICOS, OX40, Tim-3, Vista, BTLA, TDO, or TIGIT.

19. The method according to claim 21, wherein said checkpoint inhibitor is an antibody selected from the group consisting of an anti-PD-1, anti-PD-L1, anti-CTLA-4, anti-IDO, anti-KIR, anti-TIM-3, anti-LAG-3, anti-CD39, anti-CD73, anti-ICOS, anti-OX40, anti-Tim-3, anti-Vista, anti-BTLA, anti-TDO, and anti-TIGIT-antibody.

20. The method according to claim 17, wherein said chemotherapeutic agent is selected from the group consisting of an anti-microtubule agent, an platinum coordination complex and an antibiotic agent.

21. The method according to claim 17, wherein said immunotherapeutic agent is selected from the group consisting of a STING pathway modulating compound, a TLR agonist and a checkpoint inhibitor.
